(19) United States
(12) Patent Application Publication
(10) Pub. No.: US 2025/0313572 A1
(43) Pub. Date: Oct. 9, 2025

(54) MACROCYCLIC MCL-1 INHIBITORS AND METHODS OF USE

(71) Applicants: AbbVie Inc., North Chicago, IL (US); AbbVie Deutschland GmbH & Co. KG, Wiesbaden (DE)

(72) Inventors: Wilfried Braje, Wiesbaden (DE); George Doherty, Libertyville, IL (US); Katja Jantos, Wiesbaden (DE); Cheng Ji, Buffalo Grove, IL (US); Andrew Judd, Grayslake, IL (US); Aaron Kunzer, Arlington Heights, IL (US); Anthony Mastracchio, Libertyville, IL (US); Xiaohong Song, Grayslake, IL (US); Andrew Souers, Libertyville, IL (US); Gerard Sullivan, Libertyville, IL (US); Zhi-Fu Tao, Vernon Hills, IL (US); Chunqiu Lai, Libertyville, IL (US); Andreas Kling, Wiesbaden (DE); Frauke Pohlki, Wiesbaden (DE); Jessee Teske, Lake Bluff, IL (US); Michael Wendt, Vernon Hills, IL (US); Patrick Brady, Grayslake, IL (US); Xilu Wang, Libertyville, IL (US); Thomas Penning, Elmhurst, IL (US); Michael Michaelides, Libertyville, IL (US)

(21) Appl. No.: 18/952,883

(22) Filed: Nov. 19, 2024

Related U.S. Application Data
(63) Continuation of application No. 18/365,044, filed on Aug. 3, 2023, now abandoned.
Application No. 18/365,044 is a continuation of application No. 18/046,011, filed on Oct. 12, 2022, now abandoned.
Application No. 18/046,011 is a continuation of application No. 16/639,555, filed on Feb. 14, 2020, now abandoned.
(60) Provisional application No. 62/545,872, filed on Aug. 15, 2017.
(60) Provisional application No. 62/555,477, filed on Sep. 7, 2017.
(60) Provisional application No. 62/692,667, filed on Jun. 30, 2018.

(51) Int. Cl. C07D 495/16; A61P 35/00; C07D 519/00

(52) U.S. Cl. CPC C07D 495/16; A61P 35/00; C07D 519/00

(57) ABSTRACT

The present disclosure provides for compounds of Formula (I)

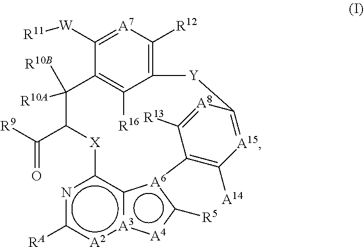

wherein A², A³, A⁴, A⁶, A⁷, A⁸, A¹⁵, R^{A}, R⁵, R⁹, R^{10A}, R^{10B}, R¹¹, R¹², R¹³, R¹⁴, R¹⁶, W, X, and Y have any of the values defined in the specification, and pharmaceutically acceptable salts thereof, that are useful as agents in the treatment of diseases and conditions, including cancer. Also provided are pharmaceutical compositions comprising compounds of Formula (I).

### CROSS REFERENCE TO RELATED APPLICATIONS

This application is a Continuation of U.S. Non-Provisional application Ser. No. 18/365,044, filed on Aug. 3, 2023, which is a Continuation of U.S. Non-Provisional application Ser. No. 18/046,011, filed on Oct. 12, 2022, which is a Continuation of U.S. Non-Provisional application Ser. No. 16/639,555, filed on Feb. 14, 2020, which is a 371 National Stage Entry of PCT Application No. PCT/US2018/000196, filed on Aug. 15, 2018, which claims the benefit of, and priority to, U.S. Provisional Application No. 62/692,677, filed on Jun. 30, 2018, which claims the benefit of, and priority to, U.S. Provisional Application No. 62/555,477, filed on Sep. 7, 2017, which claims the benefit of, and priority to, U.S. Provisional Application No. 62/545,872, filed on Aug. 15, 2017, the content of which is hereby incorporated by reference in its entirety.

### SEQUENCE LISTING

The instant application contains a sequence listing which has been submitted electronically in xml format and is hereby incorporated by reference in its entirety. Said XML copy, created on Jun. 26, 2025 is named ABV12393USC3_ST26.xml and is 1,887 bytes in size.

### BACKGROUND

### Technical Field

The present disclosure relates to inhibitors of induced myeloid leukemia cell differentiation protein (MCL-1), compositions containing compounds described herein, and methods of treatment thereof.

### Description of Related Technology

Apoptosis, a type of programmed cell death, is critical for normal development and for preservation of cellular homeostasis. Dysregulation of apoptosis is recognized to play an important role in the development of various diseases. For example, blocks in apoptotic signaling are a common requirement for oncogenesis, tumor maintenance and chemoresistance (Hanahan, D. et al. Cell 2000, 100, 57). Apoptotic pathways can be divided into two categories, intrinsic and extrinsic, depending on the origin of the death signal. The intrinsic pathway, or mitochondrial apoptotic pathway, is initiated by intracellular signals that ultimately lead to mitochondrial outer membrane permeabilization (MOMP), caspase activation and cell death.

The intrinsic mitochondrial apoptotic pathway is highly regulated, and the dynamic binding interactions between the pro-apoptotic (e.g. BAX, BAK, BAD, BIM, NOXA) and anti-apoptotic (e.g. BCL-2, BCL-XL, MCL-1) BCL-2 family members control commitment to cell death (Youle, R. J. et al. Nat. Rev. Mol. Cell Biol. 2008, 9, 47). BAK and BAX are essential mediators that upon conformational activation cause MOMP, an irreversible event that subsequently leads to cytochrome c release, caspase activation and cell death. Anti-apoptotic BCL-2 family members such as BCL-2, BCL-XL and MCL-1 can bind and sequester their pro-apoptotic counterparts, thus preventing BAX/BAK activation and promoting cell survival.

BCL-2 plays a dominant role in the survival of several hematological malignancies where it is frequently overexpressed, whereas BCL-XL is a key survival protein in some hematological and solid tumors. The related anti-apoptotic protein MCL-1 is implicated in mediating malignant cell survival in a number of primary tumor types (Ashkenazi, A. et al. Nature Rev Drug Discovery 2017, 16, 273), MCL-1 gene amplifications are frequently found in human cancers, including breast cancer and non-small cell lung cancer (Beroukhim, R. et al. Nature 2010, 463, 899), and the MCL-1 protein has been shown to mediate survival in models of multiple myeloma (Derenn, S. et al. Blood 2002, 100, 194), acute myeloid leukemia (Glaser, S. et al. Genes Dev 2012, 26, 120) and MYC-driven lymphomas (Kelly, G. et al. Genes Dev 2014, 28, 58). Specific compounds that broadly inhibit gene transcription (e.g., CDK9 inhibitors) exert their cytotoxic effects on tumor cells, at least in part, by down-regulating MCL-1 (Kotschy, A. et al. Nature 2016, 538, 477); alvocidib (Kim, W. et al. Blood 2015, 126, 1343) and dinaciclib (Gregory, G. et al. Leukemia 2015, 29, 1437) are two examples that have demonstrated clinical proof-of-concept in patients with hematological malignancies. Literature data supports a role for MCL-1 as a resistance factor to anticancer therapies such gemcitabine, vincristine and taxol (Wertz, I. E. et al. Nature 2011, 471, 110). Accordingly, there is a need in the therapeutic arts for compounds which inhibit the activity of the MCL-1 protein.

### SUMMARY

In embodiments, the present disclosure provides for compounds of Formula (I) or a pharmaceutically acceptable salt thereof,

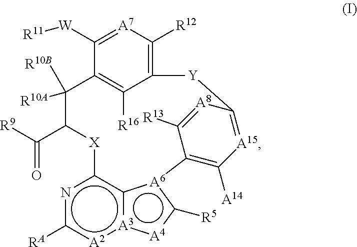

wherein
A² is CR², A³ is N, A⁴ is CR^{4a}, and A⁶ is C; or
A² is CR², A³ is N, A⁴ is O or S, and A⁶ is C; or
A² is CR², A³ is C, A⁴ is O or S and A⁶ is C; or
A² is N, A³ is C, A⁴ is O or S and A⁶ is C; or
A² is N, A³ is C, A⁴ is CR^{4a}, and A⁶ is N;
R^{A} is hydrogen, CH₃, halogen, CN, CH₂F, CHF₂, or CF₃;
X is O, or N(R^{x2}); wherein R^{x2} is hydrogen, C₁-C₃ alkyl, or unsubstituted cyclopropyl;
Y is (CH₂)ₘ, —CH═CH—(CH₂)ₙ—, —(CH₂)ₚ—CH═CH—, or —(CH₂)_{q}—CH═CH—(CH₂)—; wherein 0, 1, 2, or 3 CH₂ groups are each independently replaced by O, N(R^{ya}), C(R^{ya})(R^{yb}), C(O), NC(O)R^{ya}, or S(O)₂;
m is 2, 3, 4, or 5;
n is 1, 2, or 3;
p is 1, 2, or 3;
q is 1 or 2; and
r is 1 or 2; wherein the sum of q and r is 2 or 3;
R^{ya}, at each occurrence, is independently hydrogen, C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd}) (R^{ye}), G¹, —OR^{yf}, —SR^{yg}, —S(O)₂N(R^{yd})(R^{ye}), and —S(O)₂-G¹; and
R^{yb} is C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or
R^{ya} and R^{yb}, together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1, 2, or 3 independently selected R^{s} groups;
R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{yh}, —SR^{yh}, —SO₂R^{yh}, and —N(R^{yi})(R^{yk});
G¹, at each occurrence, is a 4-11 membered heterocycle; wherein each G¹ is optionally substituted with 1, 2, or 3 substituents independently selected from the group consisting of G², —(C₁-C₆ alkylenyl)-G², -L^{1A}-(C₁-C₆ alkylenyl); —R^{x1}, and R^{s};
G², at each occurrence, is a C₃-C₇ monocyclic cycloalkyl, C₄-C₇ monocyclic cycloalkenyl, or a 4-11 membered heterocycle; wherein each G² is optionally substituted with 1 independently selected R^{t} groups;
L^{1A} is bond, O, N(H), N(C₁-C₆ alkyl), N[(C₁-C₆ alkyl)-R^{x1}], S, S(O), or S(O)₂, C(O)NH, C(O)N(C₁-C₆ alkyl), or C(O)N[(C₁-C₆ alkyl)-R^{x1}];
R² is independently hydrogen, halogen, CH₃, or CN;
R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄ alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups;
R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³;
G³, at each occurrence, is independently C₆-C₁₀ aryl, 5-11 membered heteroaryl, C₃-C₁₁ cycloalkyl, C₄-C₁₁ cycloalkenyl, or 4-7 membered heterocycle; wherein each G³ is optionally substituted with 1, 2, or 3 R^{v} groups;
A⁷ is N or CR⁷;
A⁸ is N or CR⁸;
A¹⁵ is N or CR¹⁵;
R⁷, R¹² and R¹⁶ are each independently hydrogen, halogen, C₁-C₄ alkyl, C₁-C₄ haloalkyl, —CN, —OR^{7a}, —SR^{7a}, or —N(R^{7b})(R^{7c});
R⁸, R¹³, R¹⁴, and R¹⁵, are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; or
R⁸ and R¹³ are each independently hydrogen, halogen, C₁-C₄ alkyl, C₁-C₄ haloalkyl, —CN, —OR^{8a}, —SR^{8b}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and
R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8b})(R^{8c});
R⁹ is —OH, —O—C₁-C₄ alkyl, —O—CH₂—OC(O)(C₁-C₆ alkyl), —NHOH,

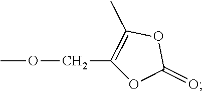

or —N(H)S(O)₂—(C₁-C₆ alkyl);
R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl; wherein the cyclopropyl is optionally substituted with one or two substituents independently selected from the group consisting of halogen, C₁-C₃ alkyl, and C₁-C₃ haloalkyl;
W is —CH═CH—, C₁-C₄ alkyl, -L¹-CHF—, -L¹-CH₂—, or —CH₂-L¹-; wherein L¹ at each occurrence, is independently O, S, S(O), S(O)₂, S(O)₂N(H), N(H), or N(C₁-C₃ alkyl);
R¹¹ is a C₆-C₁₀ aryl or a 5-11 membered heteroaryl; wherein each R¹¹ is optionally substituted with 1, 2, or 3 independently selected R^{w} groups;
R^{w}, at each occurrence, is independently C₁-C₆ alkyl, C₂-C₆ alkenyl, C₂-C₆ alkynyl, halogen, C₁-C₆ haloalkyl, —CN, NO₂, —OR^{11a}, —SR^{11b}, —S(O)₂R^{11b}, —S(O)₂N(R^{11c})₂, —C(O)R^{11a}, —C(O)N(R^{11c})₂, —N(R^{11c})₂, —N(R^{11c})C(O)R^{11b}, —N(R^{11c})S(O)₂R^{11b}, —N(R^{11c})C(O)O(R^{11b}), —N(R^{11c})C(O)N(R^{11c})₂, G⁴, —(C₁-C₆ alkylenyl)-OR^{11a}, —(C₁-C₆ alkylenyl)-OC(O)N(R^{11c})₂, —(C₁-C₆ alkylenyl)-SR^{11a}, —(C₁-C₆ alkylenyl)-S(O)₂R^{11b}, —(C₁-C₆ alkylenyl)-S(O)₂N(R^{11b})₂, —(C₁-C₆ alkylenyl)-C(O)R^{11a}, —(C₁-C₆ alkylenyl)-C(O)N(R^{11c})₂, —(C₁-C₆ alkylenyl)-N(R^{11b})₂, —(C₁-C₆ alkylenyl)-N(R^{11c})C(O)R^{11b}, —(C₁-C₆ alkylenyl)-N(R^{11c})S(O)₂R^{11b}, —(C₁-C₆ alkylenyl)-N(R^{11c})C(O)O(R^{11b}), —(C₁-C₆ alkylenyl)-N(R^{11c})C(O)N(R^{11c})₂, —(C₁-C₆ alkylenyl)-CN, —N(C₁-C₆ alkylenyl)₂-G⁴, or —(C₁-C₆ alkylenyl)-G⁴;
R^{11a} and R^{11c}, at each occurrence, are each independently hydrogen, C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11e})₂, or —(C₂-C₆ alkylenyl)-G⁴;
R^{11b}, at each occurrence, is independently C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11c})₂, or —(C₂-C₆ alkylenyl)-G⁴;
G⁴, at each occurrence, is independently R^{x1}, phenyl, monocyclic heteroaryl, C₃-C₁₁ cycloalkyl, C₄-C₁₁ cycloalkenyl, or 4-11 membered heterocycle; wherein each phenyl, monocyclic heteroaryl, C₃-C₁₁ cycloalkyl, C₄-C₁₁ cycloalkenyl, and 4-11 membered heterocycle is optionally substituted with 1, 2, 3, or 4 substituents independently selected from the group consisting of G⁵, R^{y}, —(C₁-C₆ alkylenyl)-G⁵, -L³-(C₁-C₆ alkylenyl)ₛ-R^{x1}, —(C₁-C₆ alkylenyl)ₛ-L³-(C₁-C₆ alkylenyl)ₛ-R^{x1}, -L³-(C₃-C₇ cycloalkyl)-R^{x1}, -L³-(C₄-C₇ cycloalkenyl)-R^{x1}, -L³-(4-7 membered heterocycle)-R^{x1}, and -L²-(C₁-C₆ alkylenyl)ₛ-G⁵;
L² is O, C(O), N(H), N(C₁-C₆ alkyl), NHC(O), C(O)O, S, S(O), or S(O)₂;
L³ is bond, O, C(O), N(H), N(C₁-C₆ alkyl), NHC(O), N(C₁-C₆ alkyl) C(O), N[(C₁-C₆ alkyl)ₛ-R^{x1}], N[(C₁-C₆ alkyl)ₛ-R^{x1}]C(O), S, S(O), or S(O)₂, C(O)NH, C(O)N(C₁-C₆ alkyl), or C(O)N[(C₁-C₆ alkyl)ₛ-R^{x1}];
s, at each occurrence, is independently is 0 or 1;
G⁵, at each occurrence, is independently phenyl, monocyclic heteroaryl, C₃-C₇ monocyclic cycloalkyl, C₄-C₇ monocyclic cycloalkenyl, or 4-12 membered heterocycle; wherein each G⁵ is optionally substituted with 1 independently selected R^{z} groups;
R^{s}, R^{t}, R^{u}, R^{v}, R^{y}, and R^{z}, at each occurrence, are each independently C₁-C₆ alkyl, C₂-C₆ alkenyl, C₂-C₆ alkynyl, halogen, C₁-C₆ haloalkyl, —CN, oxo, NO₂, P(O)(R^{k})₂, —OR^{m}, —OC(O)R^{k}, —OC(O)N(R^{j})₂, —SR^{j}, —S(O)₂R^{k}, —S(O)₂N(R^{j})₂, —C(O)R^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, —(C₁-C₆ alkylenyl)-OR^{j}, —(C₁-C₆ alkylenyl)-OC(O)N(R^{j})₂, —(C₁-C₆ alkylenyl)-SR^{j}, —(C₁-C₆ alkylenyl)-S(O)₂R^{k}, —(C₁-C₆ alkylenyl)-S(O)₂N(R^{j})₂, —(C₁-C₆ alkylenyl)-C(O)R^{j}, (C₁-C₆ alkylenyl)-C(O)N(R^{j})₂, —(C₁-C₆ alkylenyl)-C(O)N(R^{j})S(O)₂R^{k}, —(C₁-C₆ alkylenyl)-N(R^{j})₂, —(C₁-C₆ alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, or —(C₁-C₆ alkylenyl)-CN;
R^{m} is hydrogen, C₁-C₆ alkyl, C₁-C₆ haloalkyl, —(C₂-C₆ alkylenyl)-OR^{j}, or —(C₂-C₆ alkylenyl)-N(R^{j})₂;
R^{yh}, R^{yi}, R^{yk}, R^{7a}, R^{7b}, R^{7c}, R^{8a}, R^{8b}, R^{8c}, R^{11d}, R^{11e}, and R^{j}, at each occurrence, are each independently hydrogen, C₁-C₆ alkyl, or C₁-C₆ haloalkyl;
R^{x1}, at each occurrence, is independently selected from the group consisting of a polyethylene glycol, a polyol, a polyether, CH₂P(O)(R^{k})₂, C(O)OH, S(O)(═NH) (C₁-C₃ alkyl), a carboxylic acid isostere, C₃-C₁₁ cycloalkyl, C₄-C₁₁ cycloalkenyl, or 4-11 membered heterocycle wherein the C₃-C₁₁ cycloalkyl, C₄-C₁₁ cycloalkenyl, and 4-11 membered heterocycle are substituted with two or more ORⁿ groups and optionally substituted with 1 independently

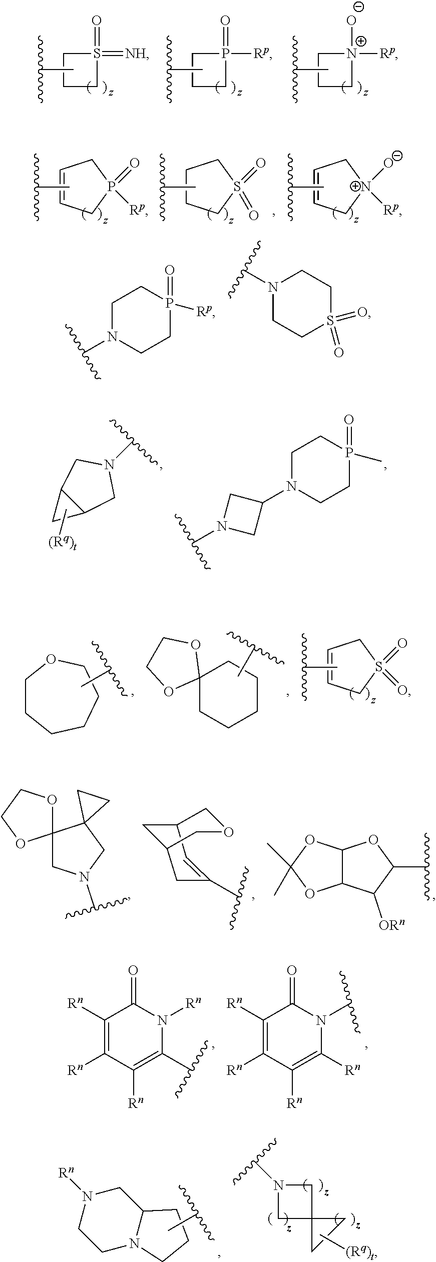

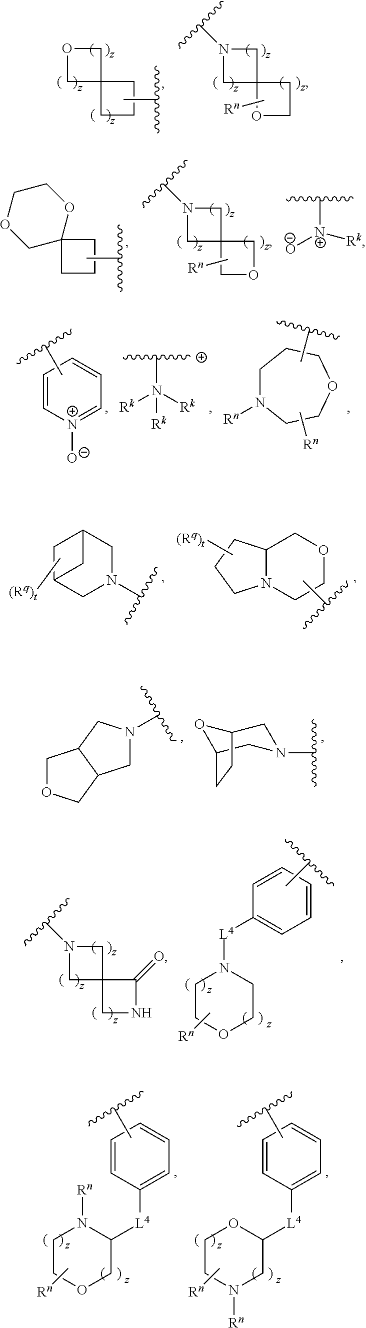

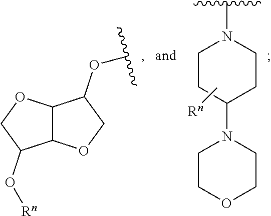

L⁴ is C₁-C₆ alkyl, —O—C₁-C₆ alkyl, C₁-C₆ alkyl-O—, C(O), N(H), N(C₁-C₆ alkyl), NHC(O), OC(O), C(O)O, or S(O)₂;
R^{k}, at each occurrence, is independently C₁-C₆ alkyl or C₁-C₆ haloalkyl;
Rⁿ, at each occurrence, is independently hydrogen, or C₁-C₆ alkyl;
R^{p} is C₁-C₃ alkyl, or cyclopropyl;
R^{q}, at each occurrence, is independently C(O)OH, —OH, halogen, —O—C₁-C₆ alkyl, or C₁-C₆ alkyl;
t is 0, 1, or 2; and
z, at each occurrence, is independently 1, 2, 3, or 4;
wherein at least one R^{x1} is present.

In embodiments, the present disclosure provides for methods of treating or preventing disorders that are amenable to inhibition of MCL-1. Such methods comprise administering to the subject a therapeutically effective amount of a compound of Formula (I), alone, or in combination with a pharmaceutically acceptable carrier.

Some of the methods are directed to treating or preventing cancer. In embodiments, the present disclosure provides for methods for treating or preventing cancer in a subject, the method comprising administering to the subject a therapeutically effective amount of a compound of Formula (I), alone, or in combination with a pharmaceutically acceptable carrier.

In embodiments, the present disclosure relates to methods of treating cancer in a subject comprising administering a therapeutically effective amount of a compound of Formula (I), or a pharmaceutically acceptable salt thereof, to a subject in need thereof. In certain embodiments, the cancer is multiple myeloma. In certain embodiments, the methods further comprise administering a therapeutically effective amount of at least one additional therapeutic agent.

In embodiments, the present disclosure provides the use of a compound of Formula (I), alone or in combination with at least one additional therapeutic agent, in the manufacture of a medicament for treating or preventing conditions and disorders disclosed herein, with or without a pharmaceutically acceptable carrier.

Pharmaceutical compositions comprising a compound of Formula (I), or a pharmaceutically acceptable salt, alone or in combination with at least one additional therapeutic agent, are also provided.

### DETAILED DESCRIPTION

In embodiments, the present disclosure provides for compounds of Formula (I), or pharmaceutically acceptable salts thereof,

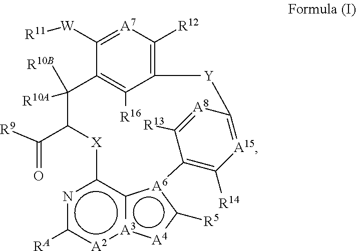

wherein
A², A³, A⁴, A⁶, A⁷, A⁸, A¹⁵, R^{A}, R⁵, R⁹, R^{10A}, R^{10B}, R¹¹, R¹², R¹³, R¹⁴, R¹⁶, W, X, and Y are defined above in the Summary and below in the Detailed Description. Further, compositions comprising such compounds and methods for treating conditions and disorders using such compounds and compositions are also included.

Compounds included herein may contain one or more variable(s) that occur more than one time in any substituent or in the formulae herein. Definition of a variable on each occurrence is independent of its definition at another occurrence. Further, combinations of substituents are permissible only if such combinations result in stable compounds. Stable compounds are compounds which can be isolated from a reaction mixture.

### Definitions

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “a pharmaceutically acceptable carrier” means a single pharmaceutically acceptable carrier as well as one or more pharmaceutically acceptable carriers, and the like.

As used in the specification and the appended claims, unless specified to the contrary, the following terms have the meaning indicated:

The term “alkenyl” as used herein, means a straight or branched hydrocarbon chain containing from 2 to 10 carbons and containing at least one carbon-carbon double bond. The term “C₂-C₆ alkenyl” and “C₂-C₄ alkenyl” means an alkenyl group containing 2-6 carbon atoms and 2-4 carbon atoms respectively. Non-limiting examples of alkenyl include buta-1,3-dienyl, ethenyl, 2-propenyl, 2-methyl-2-propenyl, 3-butenyl, 4-pentenyl, and 5-hexenyl. The terms “alkenyl,” “C₂-C₆ alkenyl,” and “C₂-C₄ alkenyl” used herein are unsubstituted, unless otherwise indicated.

The term “alkyl” as used herein, means a saturated, straight or branched hydrocarbon chain radical. In some instances, the number of carbon atoms in an alkyl moiety is indicated by the prefix “Cₓ-C_{y}”, wherein x is the minimum and y is the maximum number of carbon atoms in the substituent. Thus, for example. “C₁-C₆ alkyl” means an alkyl substituent containing from 1 to 6 carbon atoms, “C₁-C₄ alkyl” means an alkyl substituent containing from 1 to 4 carbon atoms, and “C₁-C₃ alkyl” means an alkyl substituent containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl. The terms “alkyl,” “C₁-C₆ alkyl,” “C₁-C₄ alkyl,” and “C₁-C₃ alkyl” used herein are unsubstituted, unless otherwise indicated.

The term “alkylene” or “alkylenyl” means a divalent radical derived from a straight or branched, saturated hydrocarbon chain, for example, of 1 to 10 carbon atoms or of 1 to 6 carbon atoms (C₁-C₆ alkylenyl) or of 1 to 4 carbon atoms (C₁-C₄ alkylenyl) or of 1 to 3 carbon atoms (C₁-C₃ alkylenyl) or of 2 to 6 carbon atoms (C₂-C₆ alkylenyl). Examples of alkylenyl include, but are not limited to, —CH₂—, —CH₂CH₂—, —C((CH₃)₂)—CH₂CH₂CH₂—, —C((CH₃)₂)—CH₂CH₂, —CH₂CH₂CH₂CH₂—, and —CH₂CH(CH₃)CH₂—.

The term “C₂-C₆ alkynyl” and “C₂-C₄ alkynyl” as used herein, means a straight or branched chain hydrocarbon radical containing from 2 to 6 carbon atoms and 2 to 4 carbon atoms respectively, and containing at least one carbon-carbon triple bond. Representative examples of C₂-C₆ alkynyl and C₂-C₄ alkynyl include, but are not limited, to acetylenyl, 1-propynyl, 2-propynyl, 3-butynyl, 2-pentynyl, and 1-butynyl. The terms “alkynyl,” “C₂-C₆ alkynyl,” and “C₂-C₄ alkynyl” used herein are unsubstituted, unless otherwise indicated.

The term “C₆-C₁₀ aryl” as used herein, means phenyl or a bicyclic aryl. The bicyclic aryl is naphthyl, or a phenyl fused to a C₃-C₆ monocyclic cycloalkyl, or a phenyl fused to a C₄-C₆ monocyclic cycloalkenyl. Non-limiting examples of the aryl groups include dihydroindenyl, indenyl, naphthyl, dihydronaphthalenyl, and tetrahydronaphthalenyl.

The term “C₃-C₁₁ cycloalkyl” as used herein, means a non-aromatic hydrocarbon ring radical containing 3-11 carbon atoms, zero heteroatom, and zero double bond. The C₃-C₁₁ cycloalkyl group may be a single-ring (monocyclic) or have two or more rings (polycyclic or bicyclic). Monocyclic cycloalkyl groups typically contain from 3 to 8 carbon ring atoms (C₃-C₈ monocyclic cycloalkyl) or 3 to 7 carbon ring atoms (C₃-C₇ monocyclic cycloalkyl), and even more typically 3-6 carbon ring atoms (C₃-C₆ monocyclic cycloalkyl). Examples of monocyclic cycloalkyls include cyclopropyl, cyclobutyl, cyclopentyl, cyclohexyl, cycloheptyl, and cyclooctyl. Polycyclic cycloalkyl groups contain two or more rings, and bicyclic cycloalkyls contain two rings. In certain embodiments, the polycyclic cycloalkyl groups contain 2 or 3 rings. The rings within the polycyclic and the bicyclic cycloalkyl groups may be in a bridged, fused, or spiro orientation, or combinations thereof. In a spirocyclic cycloalkyl, one atom is common to two different rings. An example of a spirocyclic cycloalkyl is spiro[4.5]decane. In a bridged cycloalkyl, the rings share at least two non-adjacent atoms. Examples of bridged cycloalkyls include, but are not limited to, bicyclo[1,1,1]pentanyl, bicyclo[2.2.2]octanyl, bicyclo[3.2.1]octanyl, bicyclo[3.1.1]heptyl, bicyclo[2.2.1]heptyl, bicyclo[3.2.2]nonyl, bicyclo[3.3.1]nonyl, bicyclo[4.2.1]nonyl, tricyclo[3.3.1.0^{3,7}]nonyl(octahydro-2,5-methanopentalenyl or noradamantyl), tricyclo[3.3.1.1^{3,7}]decyl(adamantyl), and tricyclo[4.3.1.1^{3,8}]undecyl (homoadamantyl). In a fused ring cycloalkyl, the rings share one common bond. Example of fused-ring cycloalkyl include, but not limited to, decalin (decahydronaphthyl).

The term “C₃-C₇ monocyclic cycloalkyl” as used herein, means cyclopropyl, cyclobutyl, cyclopentyl, cyclohexyl, and cycloheptyl.

The term “C₃-C₆ monocyclic cycloalkyl” as used herein, means cyclopropyl, cyclobutyl, cyclopentyl, and cyclohexyl.

The term “C₃-C₄ monocyclic cycloalkyl” as used herein, means cyclopropyl and cyclobutyl.

The term “C₄-C₇ monocyclic cycloalkenyl” as used herein, means cyclobutenyl, cyclopentenyl, cyclohexenyl, and cycloheptanyl.

The term “C₄-C₁₁ cycloalkenyl” as used herein, refers to a monocyclic or a bicyclic hydrocarbon ring radical. The monocyclic cycloalkenyl has four-, five-, six-, seven- or eight carbon atoms and zero heteroatoms. The four-membered ring systems have one double bond, the five- or six-membered ring systems have one or two double bonds, and the seven- or eight-membered ring systems have one, two, or three double bonds. Representative examples of monocyclic cycloalkenyl groups include, but are not limited to, cyclobutenyl, cyclopentenyl, cyclohexenyl, cycloheptenyl, and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group. The monocyclic and bicyclic cycloalkenyl ring may contain one or two alkylene bridges, each consisting of one, two, or three carbon atoms, and each linking two non-adjacent carbon atoms of the ring system. Representative examples of the bicyclic cycloalkenyl groups include, but are not limited to, 4,5,6,7-tetrahydro-3aH-indene, octahydronaphthalenyl, and 1,6-dihydro-pentalene. The monocyclic and the bicyclic cycloalkenyls, including exemplary rings, are optionally substituted unless otherwise indicated. The monocyclic cycloalkenyl and bicyclic cycloalkenyl are attached to the parent molecular moiety through any substitutable atom contained within the ring systems.

The term “halo” or “halogen” as used herein, means Cl, Br, I, and F.

The term “haloalkyl” as used herein, means an alkyl group, as defined herein, in which one, two, three, four, five, or six hydrogen atoms are replaced by halogen. The term “C₁-C₆ haloalkyl” means a C₁-C₆ alkyl group, as defined herein, in which one, two, three, four, five, or six hydrogen atoms are replaced by halogen. The term “C₁-C₄ haloalkyl” means a C₁-C₄ alkyl group, as defined herein, in which one, two, three, four, or five hydrogen atoms are replaced by halogen. The term “C₁-C₃ haloalkyl” means a C₁-C₃ alkyl group, as defined herein, in which one, two, three, four, or five hydrogen atoms are replaced by halogen. Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, 2-chloro-3-fluoropentyl, trifluorobutyl, and trifluoropropyl. The terms “haloalkyl.” “C₁-C₆ haloalkyl.” “C₁-C₄ haloalkyl.” and “C₁-C₃ haloalkyl.” as used herein are unsubstituted, unless otherwise indicated.

The term “5-11 membered heteroaryl” as used herein, means a monocyclic heteroaryl and a bicyclic heteroaryl. The monocyclic heteroaryl is a five- or six-membered hydrocarbon ring wherein at least one carbon ring atom is replaced by heteroatom independently selected from the group consisting of O, N, and S. The five-membered ring contains two double bonds. The five membered ring may have one heteroatom selected from O or S; or one, two, three, or four nitrogen atoms and optionally one oxygen or one sulfur atom. The six-membered ring contains three double bonds and one, two, three or four nitrogen atoms. Examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pvrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic C₃-C₆ cycloalkyl, or a monocyclic heteroaryl fused to C₄-C₆ monocyclic cycloalkenyl, or a monocyclic heteroaryl fused to a monocyclic heteroaryl, or a monocyclic heteroaryl fused to a 4-7 membered monocyclic heterocycle. Representative examples of bicyclic heteroaryl groups include, but are not limited to, benzofuranyl, benzothienyl, benzoxazolyl, benzimidazolyl, benzoxadiazolyl, phthalazinyl, 2,6-dihydropyrrolo[3,4-c]pyrazol-5(4H)-yl, 6,7-dihydro-pyrazolo[1,5-a]pyrazin-5(4H)-yl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, 2,4,6,7-tetrahydro-5H-pyrazolo[4,3-c]pyridin-5-yl, thiazolo[5,4-b]pyridin-2-yl, thiazolo[5,4-d]pyrimidin-2-yl, and 5,6,7,8-tetrahydroquinolin-5-yl. The nitrogen atom in the heteroaryl rings may optionally be oxidized and may optionally be quaternized.

The term “4-11 membered heterocycle” as used herein, means a hydrocarbon ring radical of 4-11 carbon ring atoms wherein at least one carbon ring atom is replaced by atoms independently selected from the group consisting of O, N, S, P(═O), and Si. The 4-11 membered heterocycle ring may be a single ring (monocyclic) or have two or more rings (bicyclic or polycyclic). In certain embodiments, the monocyclic heterocycle is a four-, five-, six-, or seven-, membered hydrocarbon ring wherein at least one carbon ring atom is replaced by atoms independently selected from the group consisting of O, N, S, P(═O), and Si. In certain embodiments, the monocyclic heterocycle is a 4-6 membered hydrocarbon ring wherein at least one carbon ring atom is replaced by atoms independently selected from the group consisting of O, N, S, P(═O), and Si. A four-membered monocyclic heterocycle contains zero or one double bond, and one carbon ring atom replaced by an atom selected from the group consisting of O. N. and S. A five-membered monocyclic heterocycle contains zero or one double bond and one, two, or three carbon ring atoms replaced by atoms selected from the group consisting of O, N, S, P(═O), and Si. Examples of five-membered monocyclic heterocycles include those containing in the ring: 1 O; 1 S; 1 N; 1 P(═O); 1 Si; 2 N; 3 N; 1 S and 1 N; 1 S, and 2 N; 1 O and 1 N; or 1 O and 2 N. Non limiting examples of 5-membered monocyclic heterocyclic groups include 1,3-dioxolanyl, tetrahydrofuranyl, dihydrofuranyl, tetrahydrothienyl, dihydrothienyl, imidazolidinyl, oxazolidinyl, imidazolinyl, isoxazolidinyl, isothiazolidinyl, pyrazolidinyl, pyrazolinyl, pyrrolidinyl, 2-pyrrolinyl, 3-pyrrolinyl, thiazolinyl, and thiazolidinyl. A six-membered monocyclic heterocycle contains zero, one, or two double bonds and one, two, or three carbon ring atoms replaced by heteroatoms selected from the group consisting of O, N, S, P(═O), and Si. Examples of six-membered monocyclic heterocycles include those containing in the ring: 1 P(═O); 1 Si; 1 O; 2 O; 1 S; 2 S; 1 N; 2 N; 3 N; 1 S,1 O, and 1 N; 1 S and 1 N; 1 S and 2 N; 1 S and 1 O; 1 S and 2 O; 1 O and 1 N; and 1 O and 2 N. Examples of six-membered monocyclic heterocycles include 1,3-oxazinanyl, tetrahydropyranyl, dihydropyrany, 1,6-dihydropyridazinyl, 1,2-dihydropyrimidinyl, 1,6-dihydropyrimidinyl, dioxanyl, 1,4-dithianyl, hexahydropyrimidinyl, morpholinyl, piperazinyl, piperidinyl, 1,2,3,6-tetrahydropyridinyl, tetrahydrothiopyranyl, thiomorpholinyl, thioxanyl, and trithianyl. Seven- and eight-membered monocyclic heterocycles contains zero, one, two, or three double bonds and one, two, or three carbon ring atoms replaced by heteroatoms selected from the group consisting of O. N. and S. Examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, 1,6-dihydropyridazinyl, 1,2-dihydropyrimidinyl, 1,6-dihydropyrimidinyl, hexahydropyrimidinyl, imidazolinyl, imidazolidinyl, isoindolinyl, isothiazolinyl, isothiazolidinyl, isoxazolinyl, isoxazolidinyl, morpholinyl, oxadiazolinyl, oxadiazolidinyl, 1,3-oxazinanyl, oxazolinyl, 1,3-oxazolidinyl, oxetanyl, piperazinyl, piperidinyl, pyranyl, pyrazolinyl, pyrazolidinyl, pyrrolinyl, pyrrolidinyl, 1,2-dihydropyridinyl, tetrahydrofuranyl, tetrahydropyridinyl, tetrahydropyrimidinyl, tetrahydropyranyl, tetrahydrothienyl, thiadiazolinyl, thiadiazolidinyl, thiazolinyl, thiazolidinyl, thiomorpholinyl, thiopyranyl, and trithianyl. Polycyclic heterocycle groups contain two or more rings, and bicyclic heterocycles contain two rings. In certain embodiments, the polycyclic heterocycle groups contain 2 or 3 rings. The rings within the polycyclic and the bicyclic heterocycle groups are in a bridged, fused, or spiro orientation, or combinations thereof. In a spirocyclic heterocycle, one atom is common to two different rings. Non limiting examples of spirocyclic heterocycles include 4,6-diazaspiro[2,4]heptanyl, 6-azaspiro[3,4]octane, 2-oxa-6-azaspiro[3,4]octan-6-yl, and 2,7-diazaspiro[4,4]nonane. In a fused ring heterocycle, the rings share one common bond. Examples of fused bicyclic heterocycles are a 4-6 membered monocyclic heterocycle fused to a phenyl group, or a 4-6 membered monocyclic heterocycle fused to a monocyclic C₃-C₆ cycloalkyl, or a 4-6 membered monocyclic heterocycle fused to a C₄-C₆ monocyclic cycloalkenyl, or a 4-6 membered monocyclic heterocycle fused to a 4-6 membered monocyclic heterocycle. Examples of fused bicyclic heterocycles include, but are not limited to hexahydropyrano[3,4-c][1,4]oxazin-1(5H)-yl, hexahydropyrrolo[3,4-c]pyrrol-2(1H)-yl, hexahydro-1H-imidazo[5,1-c][1,4]oxazinyl, hexahydro-1H-pyrrolo[1,2-c]imidazolyl, hexahydrocyclopenta[c]pyrrol-3a(1H)-yl, and 3-azabicyclo[3.1.0]hexanyl. In a bridged heterocycle, the rings share at least two non-adjacent atoms. Examples of such bridged heterocycles include, but are not limited to, azabicyclo[2.2.1]heptyl (including 2-azabicyclo[2.2.1]hept-2-yl), 8-azabicyclo[3.2.1]oct-8-yl, octahydro-2,5-epoxypentalene, hexahydro-1H-1,4-methanocyclopenta[c]furan, aza-admantane (1-azatricyclo[3.3.1.1^{3,7}]decane), and oxa-adamantane (2-oxatricyclo[3.3.1.1^{3,7}]decane). The nitrogen and sulfur heteroatoms in the heterocycle rings may optionally be oxidized (e.g. 1,1-dioxidotetrahydrothienyl, 1,1-dioxido-1,2-thiazolidinyl, 1,1-dioxidothiomorpholinyl)) and the nitrogen atoms may optionally be quaternized.

The term “4-7 membered monocyclic heterocycle” as used herein, means a four-, five-, six-, or seven-membered monocyclic heterocycle, as defined herein above.

The phenyl, the aryls, the cycloalkyls, the cycloalkenyls, the heteroaryls, and the heterocycles, including the exemplary rings, are optionally substituted unless otherwise indicated; and are attached to the parent molecular moiety through any substitutable atom contained within the ring system.

The term “heteroatom” as used herein, means a nitrogen, oxygen, and sulfur.

The term “oxo” as used herein, means a ═O group.

The term “radiolabel” as used herein, means a compound of the present disclosure in which at least one of the atoms is a radioactive atom or a radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F, ³⁵S, ¹²³I, and ¹²⁵I.

The term “polyethylene glycol” as used herein, means an oligomer or polymer which contains two or more ethylene glycol (ethane-1,2-diol) units. The “polyethylene glycol” may be terminated or capped by moieties such as, but not limited to, hydrogen, C₁-C₆ alkyl or heterocycles. Thus, “polyethylene glycol” may be represented schematically by, but is not limited to,

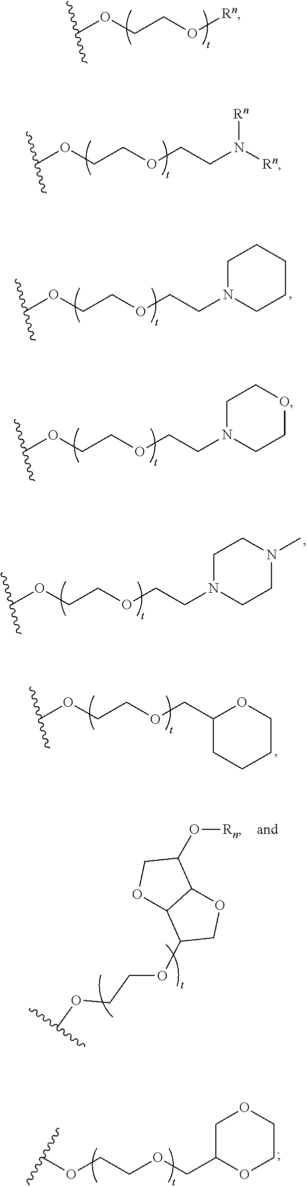

wherein t is an integer from 2-10; and Rⁿ is hydrogen or C₁C₆ alkyl. The term “polyethylene glycol” also includes crown ethers and azacrown ethers, wherein one or more oxygen atoms in a crown ether is replaced by NH. Examples of crown ether and azacrown ether moieties include, but are not limited to:

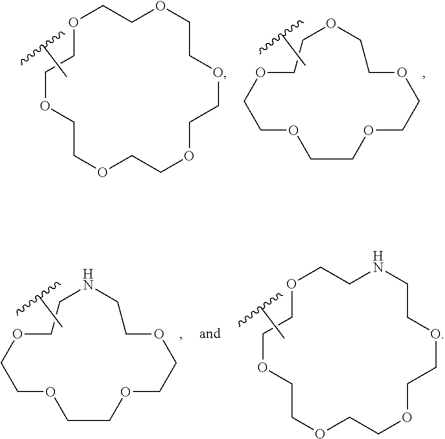

The term “polyol” as used herein, means a linear or branched carbon alkyl chain substituted by two or more hydroxyl (—OH) groups. Examples of polyol moieties include, but are not limited to:

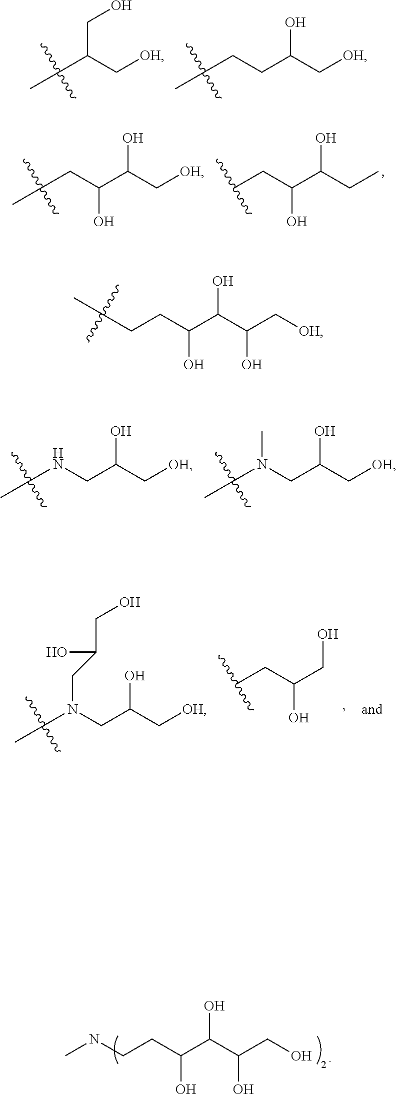

The term “polyether” as used herein, means a linear or branched carbon alkyl chain substituted by two or more alkoxyl [—O—(C₁-C₆ alkyl) groups. Examples of polyether moieties include, but are not limited to:

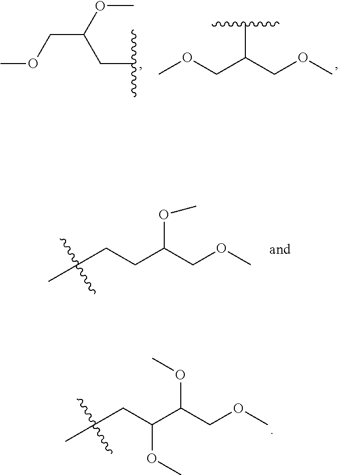

The term “carboxylic acid bioisostere” as used herein, means a group or moiety that has chemical and physical similarities to a carboxylic acid group, resulting in broadly similar biological effects. Examples of carboxylic acid bioisosteres are known in the art (Ballatore, D. ChemMedChem 2013, 8 (3), 385-395 for example) and include, but are not limited to, the following: tetrazole, phosphonic acid, phosphinic acid, hydroxamic acid, acylsulfonamide, acylsulfonylurea, 5-oxo-1,2,4-oxadiazole, 5-oxo-1,2,4-thiadiazole, thiazolidinedione, oxazolidinedione, oxadiazolidine-dione, 3-hydroxyisoxazole, 3-hydroxyisothiazole, squaric acid, and cyclic sulfonimidamide.

A moiety is described as “substituted” when a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

The terms “treat”, “treating”, and “treatment” refer to a method of alleviating or abrogating a disease and/or its attendant symptoms. In certain embodiments, “treat,” “treating.” and “treatment” refer to ameliorating at least one physical parameter, which may not be discernible by the subject. In yet another embodiment, “treat”, “treating”, and “treatment” refer to modulating the disease or disorder, either physically (for example, stabilization of a discernible symptom), physiologically (for example, stabilization of a physical parameter), or both. In a further embodiment, “treat”, “treating”, and “treatment” refer to slowing the progression of the disease or disorder.

The terms “prevent”, “preventing”, and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring or developing a disease or disorder.

The phrase “therapeutically effective amount” means an amount of a compound, or a pharmaceutically acceptable salt thereof, sufficient to prevent the development of or to alleviate to some extent one or more of the symptoms of the condition or disorder being treated when administered alone or in conjunction with another therapeutic agent for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The term “subject” is defined herein to refer to animals such as mammals, including, but not limited to, primates (e.g., humans), cows, sheep, goats, pigs, horses, dogs, cats, rabbits, rats, mice and the like. In one embodiment, the subject is a human. The terms “human,” “patient,” and “subject” are used interchangeably herein.

### Compounds

Compounds of the present disclosure have the general Formula (I) as described above.

Particular values of variable groups are as follows. Such values may be used where appropriate with any of the other values, definitions, claims or embodiments defined hereinbefore or hereinafter.

### Formula (I)

One embodiment pertains to compounds of Formula (I), or pharmaceutically acceptable salts thereof,

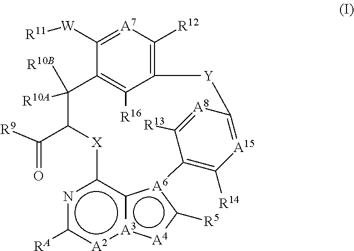

wherein
A² is CR², A³ is N, A⁴ is CR^{4a}, and A⁶ is C; or
A² is CR², A³ is N, A⁴ is O or S, and A⁶ is C; or
A² is CR², A³ is C, A⁴ is O or S and A⁶ is C; or
A² is N, A³ is C, A⁴ is O or S and A⁶ is C; or
A² is N, A³ is C, A⁴ is CR^{4a}, and A⁶ is N;
R^{A} is hydrogen, CH₃, halogen, CN, CH₂F, CHF₂, or CF₃;
X is O, or N(R^{x2}); wherein R^{x2} is hydrogen, C₁-C₃ alkyl, or unsubstituted cyclopropyl;
Y is (CH₂)ₘ, —CH═CH—(CH₂)ₙ—, —(CH₂)ₚ—CH═CH—, or —(CH₂)_{q}—CH═CH—(CH₂)ᵣ—; wherein 0, 1, 2, or 3 CH₂ groups are each independently replaced by O, N(R^{ya}), C(R^{ya})(R^{yb}), C(O), NC(O)R^{ya}, or S(O)₂;
m is 2, 3, 4, or 5;
n is 1, 2, or 3;
p is 1, 2, or 3;
q is 1 or 2; and
r is 1 or 2; wherein the sum of q and r is 2 or 3;
R^{ya}, at each occurrence, is independently hydrogen, C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; and
R^{yb} is C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or
R^{ya} and R^{yb}, together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1, 2, or 3 independently selected R^{s} groups;
R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{yh}, —SR^{yh}, —SO₂R^{yh}, and —N(R^{yi})(R^{yk});
G¹, at each occurrence, is a 4-11 membered heterocycle; wherein each G¹ is optionally substituted with 1, 2, or 3 substituents independently selected from the group consisting of G², —(C₁-C₆ alkylenyl)-G², -L^{1A}-(C₁-C₆ alkylenyl)ₛ-R^{x1}, and R^{s};
G², at each occurrence, is a C₃-C₇ monocyclic cycloalkyl, C₄-C₇ monocyclic cycloalkenyl, or a 4-11 membered heterocycle; wherein each G² is optionally substituted with 1 independently selected R¹ groups;
L^{1A} is bond, O, N(H), N(C₁-C₆ alkyl), N[(C₁-C₆ alkyl)-R^{x1}], S, S(O), or S(O)₂, C(O)NH, C(O)N(C₁-C₆ alkyl), or C(O)N[(C₁-C₆ alkyl)-R^{x1}];
R² is independently hydrogen, halogen, CH₃, or CN;
R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄ alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups;
R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³;
G³, at each occurrence, is independently C₆-C₁₀ aryl, 5-11 membered heteroaryl, C₃-C₁₁ cycloalkyl, C₄-C₁₁ cycloalkenyl, or 4-7 membered heterocycle; wherein each G³ is optionally substituted with 1, 2, or 3 R^{v} groups;
A⁷ is N or CR⁷;
A⁸ is N or CR⁸;
A¹⁵ is N or CR¹⁵;
R⁷, R¹² and R¹⁶ are each independently hydrogen, halogen, C₁-C₄ alkyl, C₁-C₄ haloalkyl, —CN, —OR^{7a}, —SR^{7a}, or —N(R^{7b}) (R^{7c});
R⁸, R¹³, R¹⁴, and R¹⁵, are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; or
R⁸ and R¹³ are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and
R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8b})(R^{8c});
R⁹ is —OH, —O—C₁-C₄ alkyl, —O—CH₂—OC(O)(C₁-C₆ alkyl), —NHOH,

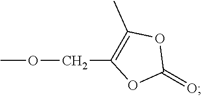

or —N(H)S(O)₂—(C₁-C₆ alkyl);
R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl; wherein the cyclopropyl is optionally substituted with one or two substituents independently selected from the group consisting of halogen, C₁-C₃ alkyl, and C₁-C₃ haloalkyl;
W is —CH═CH—, C₁-C₄ alkyl, -L¹-CHF—, -L¹-CH₂—, or —CH₂-L¹-; wherein L¹ at each occurrence, is independently O, S, S(O), S(O)₂, S(O)₂N(H), N(H), or N(C₁-C₃ alkyl);
R¹¹ is a C₆-C₁₀ aryl or a 5-11 membered heteroaryl; wherein each R¹¹ is optionally substituted with 1, 2, or 3 independently selected R^{w} groups;
R^{w}, at each occurrence, is independently C₁-C₆ alkyl, C₂-C₆ alkenyl, C₂-C₆ alkynyl, halogen, C₁-C₆ haloalkyl, —CN, NO₂, —OR^{11a}, —SR^{11b}, —S(O)₂R^{11b}, —S(O)₂N(R^{11c})₂, —C(O)R^{11a}, —C(O)N(R^{11c})₂, —N(R^{11c})₂, —N(R^{11c}) C(O)R^{11b}, —N(R^{11c}) S(O)₂R^{11b}, —N(R^{11c})C(O)O(R^{11b}), —N(R^{11c})C(O)N(R^{11c})₂, G⁴, —(C₁-C₆ alkylenyl)-OR^{11a}, —(C₁-C₆ alkylenyl)-OC(O)N(R^{11c})₂, —(C₁-C₆ alkylenyl)-SR^{11a}, —(C₁-C₆ alkylenyl)-S(O)₂R^{11b}, —(C₁-C₆ alkylenyl)-S(O)₂N(R^{11c})₂, —(C₁-C₆ alkylenyl)-C(O)R^{11a}, —(C₁-C₆ alkylenyl)-C(O)N(R^{11c})₂, —(C₁-C₆ alkylenyl)-N(R^{11c})₂, —(C₁-C₆ alkylenyl)-N(R^{11c})C(O)R^{11b}, —(C₁-C₆ alkylenyl)-N(R^{11c})S(O)₂R^{11b}, —(C₁-C₆ alkylenyl)-N(R^{11c})C(O)O(R^{11b}), —(C₁-C₆ alkylenyl)-N(R^{11c})C(O)N(R^{11c})₂, —(C₁-C₆ alkylenyl)-CN, —N(C₁-C₆ alkylenyl)₂-G⁴, or —(C₁-C₆ alkylenyl)-G⁴;
R^{11a} and R^{11e}, at each occurrence, are each independently hydrogen, C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11e})₂, or —(C₂-C₆ alkylenyl)-G⁴;
R^{11b}, at each occurrence, is independently C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11e})₂, or —(C₂-C₆ alkylenyl)-G⁴;
G⁴, at each occurrence, is independently R^{x1}, phenyl, monocyclic heteroaryl, C₃-C₁₁ cycloalkyl, C₄-C₁₁ cycloalkenyl, or 4-11 membered heterocycle; wherein each phenyl, monocyclic heteroaryl, C₃-C₁₁ cycloalkyl, C₄-C₁₁ cycloalkenyl, and 4-11 membered heterocycle is optionally substituted with 1, 2, 3, or 4 substituents independently selected from the group consisting of G⁵, R^{y}, —(C₁-C₆ alkylenyl)-G⁵, -L³-(C₁-C₆ alkylenyl)ₛ-R^{x1}, —(C₁-C₆ alkylenyl)ₛ-L³-(C₁-C₆ alkylenyl)ₛ-R^{x1}, -L³-(C₃-C₇ cycloalkyl)-R^{x1}, -L³-(C₄-C₇ cycloalkenyl)-R^{x1}, -L³-(4-7 membered heterocycle)-R^{x1}, and -L²-(C₁-C₆ alkylenyl)ₛ-G⁵;
L² is O, C(O), N(H), N(C₁-C₆ alkyl), NHC(O), C(O)O, S, S(O), or S(O)₂;
L³ is bond, O, C(O), N(H), N(C₁-C₆ alkyl), NHC(O), N(C₁-C₆ alkyl)C(O), N[(C₁-C₆ alkyl)ₛ-R^{x1}], N[(C₁-C₆ alkyl)ₛ-R^{x1}]C(O), S, S(O), or S(O)₂, C(O)NH, C(O)N(C₁-C₆ alkyl), or C(O)N[(C₁-C₆ alkyl)ₛ-R^{x1}];
s, at each occurrence, is independently is 0 or 1;
G⁵, at each occurrence, is independently phenyl, monocyclic heteroaryl, C₃-C₇ monocyclic cycloalkyl, C₄-C₇ monocyclic cycloalkenyl, or 4-12 membered heterocycle; wherein each G⁵ is optionally substituted with 1 independently selected R² groups;
R^{s}, R^{t}, R^{u}, R^{v}, R^{y}, and R^{z}, at each occurrence, are each independently C₁-C₆ alkyl, C₂-C₆ alkenyl, C₂-C₆ alkynyl, halogen, C₁-C₆ haloalkyl, —CN, oxo, NO₂, P(O)(R*)₂, —OR^{m}, —OC(O)R^{k}, —OC(O)N(R^{j})₂, —SR^{j}, —S(O)₂R^{k}, —S(O)₂N(R^{j})₂, —C(O)R^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, —(C₁-C₆ alkylenyl)-OR^{j}, —(C₁-C₆ alkylenyl)-OC(O)N(R^{j})₂, —(C₁-C₆ alkylenyl)-SR^{j}, —(C₁-C₆ alkylenyl)-S(O)₂R^{k}, —(C₁-C₆ alkylenyl)-S(O)₂N(R^{j})₂, —(C₁-C₆ alkylenyl)-C(O)R^{j}, —(C₁-C₆ alkylenyl)-C(O)N(R^{j})₂, —(C₁-C₆ alkylenyl)-C(O)N(R^{j})S(O)₂R^{k}, —(C₁-C₆ alkylenyl)-N(R^{j})₂, —(C₁-C₆ alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, or —(C₁-C₆ alkylenyl)-CN;
R^{m} is hydrogen, C₁-C₆ alkyl, C₁-C₆ haloalkyl, —(C₂-C₆ alkylenyl)-OR^{j}, or —(C₂-C₆ alkylenyl)-N(R^{j})₂;
R^{yh}, R^{yi}, R^{yk}, R^{7a}, R^{7b}, R^{7c}, R^{8a}, R^{8b}, R^{8c}, R^{11d}, R^{11e}, and R^{j}, at each occurrence, are each independently hydrogen, C₁-C₆ alkyl, or C₁-C₆ haloalkyl;
R^{x1}, at each occurrence, is independently selected from the group consisting of a polyethylene glycol, a polyol, a polyether, CH₂P(O)(R^{k})₂, C(O)OH, S(O)(═NH) (C₁-C₃ alkyl), a carboxylic acid isostere, C₃-C₁₁ cycloalkyl, C₄-C₁₁ cycloalkenyl, or 4-11 membered heterocycle wherein the C₃-C₁₁ cycloalkyl, C₄-C₁₁ cycloalkenyl, and 4-11 membered heterocycle are substituted with two or more ORⁿ groups and optionally substituted with 1 independently selected R^{z} group,

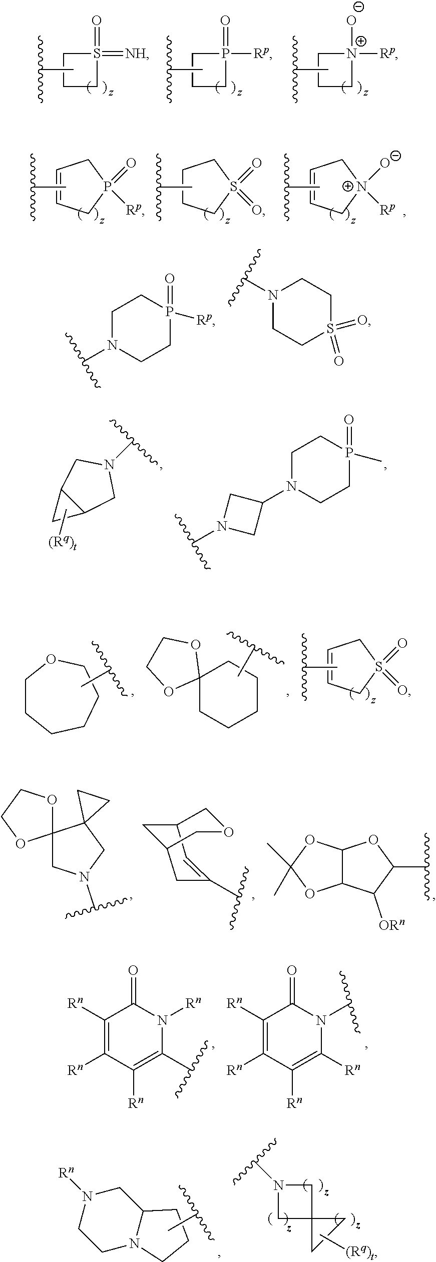

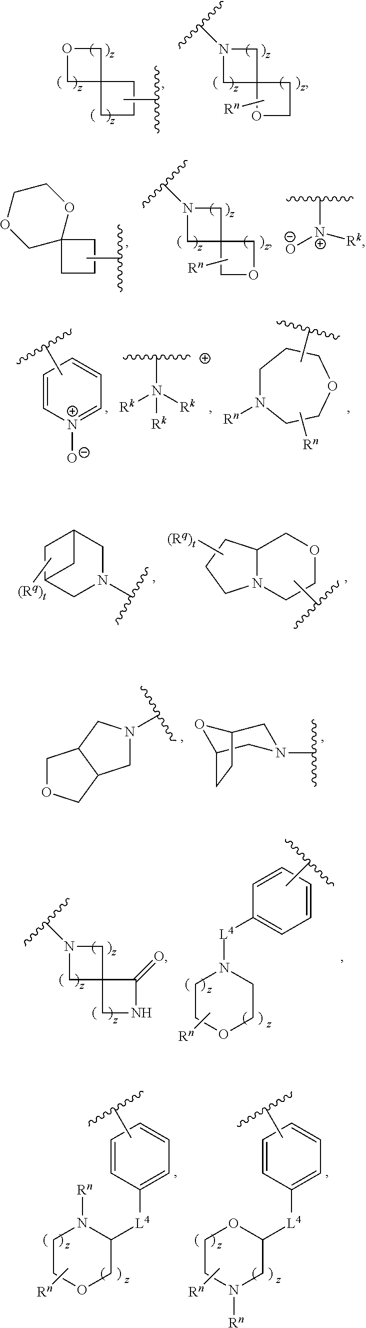

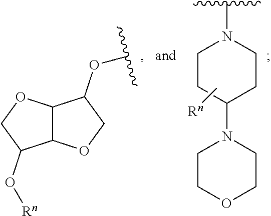

L⁴ is C₁-C₆ alkyl, —O—C₁-C₆ alkyl, C₁-C₆ alkyl-O—, C(O), N(H), N(C₁-C₆ alkyl), NHC(O), OC(O), C(O)O, or S(O)₂;
R^{k}, at each occurrence, is independently C₁-C₆ alkyl or C₁-C₆ haloalkyl;
Rⁿ, at each occurrence, is independently hydrogen, or C₁-C₆ alkyl;
R^{p} is C₁-C₃ alkyl, or cyclopropyl;
R^{q}, at each occurrence, is independently C(O)OH, —OH, halogen, —O—C₁-C₆ alkyl, or C₁-C₆ alkyl;
t is 0, 1, or 2; and
z, at each occurrence, is independently 1, 2, 3, or 4;
wherein at least one R^{x1} is present.

In one embodiment of Formula (I), A² is CR², A³ is N, A⁴ is CR^{4a}, and A⁶ is C; or A² is CR², A³ is N, A⁴ is O or S, and A⁶ is C; or A² is CR², A³ is C, A⁴ is O or S and A⁶ is C; or A² is N, A³ is C, A⁴ is O or S and A⁶ is C; or A² is N, A³ is C, A⁴ is CR^{4a}, and A⁶ is N. In another embodiment of Formula (I), A² is CR², A³ is N, A⁴ is CR^{4a}, and A⁶ is C. In another embodiment of Formula (I), A² is CH, A³ is N, A⁴ is CH, and A⁶ is C. In another embodiment of Formula (I), A² is CR², A³ is N, A⁴ is CR^{4a}, A⁶ is C, R² is H, and R^{4a} is halogen. In another embodiment of Formula (I), A² is CR², A³ is N, A⁴ is CR^{4a}, A⁶ is C, R² is H, and R^{4a} is Cl. In another embodiment of Formula (I), A² is CR², A³ is N, A⁴ is O or S, and A⁶ is C. In another embodiment of Formula (I), A² is N, A³ is C, A⁴ is O, and A⁶ is C. In another embodiment of Formula (I), A² is N, A³ is C, A⁴ is S, and A⁶ is C. In another embodiment of Formula (I), A² is N, A³ is C, A⁴ is CR^{4a}, and A⁶ is N. In another embodiment of Formula (I), A² is CR², A³ is C, A⁴ is O or S and A⁶ is C.

In one embodiment of Formula (I), R^{A} is hydrogen, CH₃, halogen, CN, CH₂F, CHF₂, or CF₃. In another embodiment of Formula (I), R^{A} is hydrogen.

In one embodiment of Formula (I), X is O, or N(R^{x2}); wherein R^{x2} is hydrogen, C₁-C₃ alkyl, or unsubstituted cyclopropyl. In another embodiment of Formula (I), X is O.

In one embodiment of Formula (I), Y is (CH₂)ₘ, —CH═CH—(CH₂)ₙ—, —(CH₂)ₚ—CH═CH—, or —(CH₂)_{q}—CH═CH—(CH₂)ᵣ—; wherein 0, 1, 2, or 3 CH₂ groups are each independently replaced by O, N(R^{ya}), C(R^{ya})(R^{yb}), C(O), NC(O)R^{ya}, or S(O)₂; and m is 2, 3, 4, or 5. In another embodiment of Formula (I), Y is (CH₂)ₘ; wherein 1, 2, or 3 CH₂ groups are each independently replaced by O, N(R^{ya}), C(R^{ya})(R^{yb}), C(O), or NC(O)R^{ya}; and m is 3 or 4. In another embodiment of Formula (I), Y is (CH₂)ₘ; wherein 1 CH₂ group is independently replaced by N(R^{ya}); and m is 3. In another embodiment of Formula (I), Y is (CH₂)ₘ; wherein 2 CH₂ groups are each independently replaced by O and 1 CH₂ group is replaced by C(R^{ya})(R^{yb}); and m is 4. In another embodiment of Formula (I), Y is

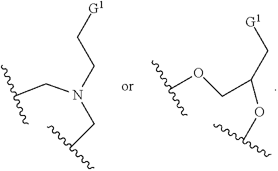

In another embodiment of Formula (I), Y is

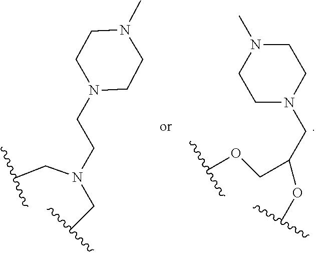 [SMILES: *CN(C*)CCN1CCN(C)CC1 ; *OCC(CN1CCN(C)CC1)O*]

In one embodiment of Formula (I), R^{ya}, at each occurrence, is independently hydrogen, C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; and R^{yb} is C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or R^{ya} and R^{yb}, together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1 —OR^{m} and 0, 1, 2, or 3 independently selected R^{s} groups; and R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{yh}, —SR^{yh}, —SO₂R^{yh}, and —N(R^{yi})(R^{yk}). In another embodiment of Formula (I), R^{ya}, at each occurrence, is independently hydrogen, or C₁-C₆ alkyl; wherein the C₁-C₆ alkyl is optionally substituted with 1 or 2 G¹; and R^{yb} is C₁-C₆ alkyl; wherein the C₁-C₆ alkyl is optionally substituted with 1 or 2 G¹. In another embodiment of Formula (I), R^{ya}, at each occurrence, is independently hydrogen; and R^{yb} is C₁-C₆ alkyl; wherein the C₁-C₆ alkyl is substituted with 1 G¹.

In one embodiment of Formula (I), G¹, at each occurrence, is 4-11 membered heterocycle; wherein each G¹ is optionally substituted with 1, 2, or 3 substituents independently selected from the group consisting of G², —(C₁-C₆ alkylenyl)-G², -L^{1A}-(C₁-C₆ alkylenyl)-R^{x1}, and R^{s}. In another embodiment of Formula (I), G¹ is piperazinyl optionally substituted with 1, 2, or 3 substituents independently selected from the group consisting of G², —(C₁-C₆ alkylenyl)-G², -L^{1A}-(C₁-C₆ alkylenyl)ₛ-R^{x1}, and R^{s}. In another embodiment of Formula (I). G¹ is piperazinyl substituted with 1 R^{s}. In another embodiment of Formula (I). G¹ is piperazinyl substituted with 1 R^{s}; and R^{s} is C₁-C₆ alkyl. In another embodiment of Formula (I). G¹ is piperazinyl substituted with 1 R^{s}; and R^{s} is CH₃. In another embodiment of Formula (I). G¹ is piperazinyl substituted with -L^{1A}-(C₁-C₆ alkylenyl)ₛ-R^{x1}. In another embodiment of Formula (I). G¹ is piperazinyl substituted with 1-L^{1A}-(C₁-C₆ alkylenyl)ₛ-R^{x1}; L^{1A} is bond; s is 0 or 1; and R^{x1} is a polyethylene glycol, or 4-11 membered heterocycle substituted with two or more ORⁿ groups. In another embodiment of Formula (I), G¹ is piperazinyl substituted with 1-L^{1A}-(C₁-C₆ alkylenyl)ₛ-R^{x1}; L^{1A} is bond; s is 0 or 1; R^{x1} is a polyethylene glycol, or 4-11 membered heterocycle substituted with two or more ORⁿ groups; and Rⁿ, at each occurrence, is independently hydrogen, or C₁-C₆ alkyl.

In one embodiment of Formula (I), G², at each occurrence, is a C₃-C₇ monocyclic cycloalkyl, C₄-C₇ monocyclic cycloalkenyl, or a 4-11 membered heterocycle: wherein each G² is optionally substituted with 1 independently selected R^{t} groups. In another embodiment of Formula (I), G², at each occurrence, is a C₃-C₇ monocyclic cycloalkyl.

In one embodiment of Formula (I), L^{1A} is bond, O, N(H), N(C₁-C₆ alkyl), N[(C₁-C₆ alkyl)-R^{x1}], S, S(O), or S(O)₂, C(O)NH, C(O)N(C₁-C₆ alkyl), or C(O)N[(C₁-C₆ alkyl)-R^{x1}], In another embodiment of Formula (I), L^{1A} is bond.

In one embodiment of Formula (I), R² is independently hydrogen, halogen, CH₃, or CN. In another embodiment of Formula (I), R² is independently hydrogen.

In one embodiment of Formula (I), R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄ alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups. In another embodiment of Formula (I), R^{4a}, at each occurrence, is independently halogen.

In one embodiment of Formula (I), R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³; and G³, at each occurrence, is independently C₆-C₁₀ aryl, 5-11 membered heteroaryl, C₃-C₁₁ cycloalkyl, C₄-C₁₁ cycloalkenyl, oxetanyl, or 2-oxaspiro[3.3]heptanyl; wherein each G³ is optionally substituted with 1, 2, or 3 R^{v} groups. In another embodiment of Formula (I), R⁵ is independently hydrogen, G³, or C₂-C₆ alkynyl; and G³, at each occurrence, is independently C₆-C₁₀ aryl, or C₃-C₁₁ cycloalkyl: wherein each G³ is optionally substituted with 1, 2, or 3 R^{v} groups. In another embodiment of Formula (I), R⁵ is independently hydrogen, G³, or C₂-C₆ alkynyl; and G³, at each occurrence, is independently C₆-C₁₀ aryl, C₄-C₁₁ cycloalkenyl, or C₃-C₁₁ cycloalkyl; wherein each G³ is optionally substituted with 1, 2, or 3 R^{v} groups.

In another embodiment of Formula (I), R⁵ is independently G³; and G³, at each occurrence, is independently C₄-C₁₁ cycloalkenyl; which is unsubstituted. In another embodiment of Formula (I), R⁵ is independently G³; and G³, at each occurrence, is independently C₃-C₁₁ cycloalkyl; which is unsubstituted. In another embodiment of Formula (I), R⁵ is independently G³; and G³, at each occurrence, is independently C₆-C₁₀ aryl; wherein each G³ is optionally substituted with 1 R^{v} groups. In another embodiment of Formula (I), R⁵ is independently G³; and G³, at each occurrence, is independently phenyl: wherein each G³ is optionally substituted with 1 R^{v} groups; and R^{v} is halogen. In another embodiment of Formula (I), R⁵ is independently G³; and G³, at each occurrence, is independently phenyl: wherein G³ is optionally substituted with 1 R^{v} groups; and R^{v} is Cl.

In one embodiment of Formula (I), A⁷ is N or CR⁷; A⁸ is N or CR⁸; and A¹⁵ is N or CR¹⁵. In another embodiment of Formula (I), R⁷, R¹² and R¹⁶ are each independently hydrogen, halogen, C₁-C₄ alkyl, C₁-C₄ haloalkyl, —CN, —OR^{7a}, —SR^{7a}, or —N(R^{7b})(R^{7c}); and R⁸, R¹³, R¹⁴, and R¹⁵, are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl. In another embodiment of Formula (I), R¹, R¹² and R¹⁶ are each independently hydrogen. In another embodiment of Formula (I), A⁷ is CH; A⁸ is CR⁸; and A¹⁵ is CR¹⁵; and R⁸, and R¹⁵ are each independently hydrogen, halogen, or C₁-C₄ alkyl. In another embodiment of Formula (I), A⁷ is CH; A⁸ is CR⁸; and A¹⁵ is CR¹⁵; and R⁸ and R¹⁵ are each independently hydrogen, halogen, C₁-C₄ alkyl, or —OR^{8a}.

In one embodiment of Formula (I), R⁸ and R¹³ are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8b})(R^{8c}). In another embodiment of Formula (I), R⁸ and R¹³ are each independently hydrogen, and R¹⁴ and R¹⁵, together with the carbon atoms to which they are attached form benzene.

In one embodiment of Formula (I), R⁹ is —OH, —O—C₁-C₄ alkyl, —O—CH₂—OC(O)(C₁-C₆ alkyl). —NHOH,

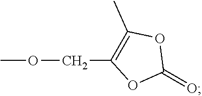

or —N(H)S(O)₂—(C₁-C₆ alkyl). In another embodiment of Formula (I), R⁹ is —OH.

In one embodiment of Formula (I), R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl: wherein the cyclopropyl is optionally substituted with one or two substituents independently selected from the group consisting of halogen and CH₃. In another embodiment of Formula (I), R^{10A} and R^{10B} are each independently hydrogen.

In one embodiment of Formula (I).
R^{A} is hydrogen;
R⁹ is —OH;
R^{10A} and R^{10B}, are each independently hydrogen; and
R⁷, R¹² and R¹⁶ are each independently hydrogen.

In one embodiment of Formula (I), W is —CH═CH—, C₁-C₄ alkyl, —O—CHF—, -L¹-CH₂—, or —CH₂-L¹-; wherein L¹ at each occurrence, is independently O, S, S(O), S(O)₂, S(O)₂N(H), N(H), or N(C₁-C₃ alkyl). In another embodiment of Formula (I), W is —O—CHF—, or -L¹-CH₂—; wherein L¹ at each occurrence, is independently O. In another embodiment of Formula (I), W is -L¹-CH₂—; wherein L¹ at each occurrence, is independently O.

In one embodiment of Formula (I), R¹¹ is a C₆-C₁₀ aryl or a 5-11 membered heteroaryl; wherein each R¹¹ is optionally substituted with 1, 2, or 3 independently selected R^{w} groups. In another embodiment of Formula (I), R¹¹ is a C₆-C₁₀ aryl or a 5-11 membered heteroaryl; wherein each R¹¹ is optionally substituted with 1 or 2 independently selected R^{w} groups. In another embodiment of Formula (I), W is —O—CH₂—, and R¹¹ is pyrimidinyl, optionally substituted with 1, 2, or 3 independently selected R^{w} groups.

In another embodiment of Formula (I), W is —O—CH₂—; and R¹¹ is pyrimidinyl, optionally substituted with 1 independently selected R^{w} groups; and R^{w}, at each occurrence, is independently —OR^{11a}, -G⁴, —N(C₁-C₆ alkylenyl)₂-G⁴, or —(C₁-C₆ alkylenyl)-G⁴. In another embodiment of Formula (I), W is —O—CH₂—; and R¹¹ is pyrimidinyl, optionally substituted with 1 independently selected R^{w} groups; and R^{w}, at each occurrence, is independently —OR^{11a}. In another embodiment of Formula (I), W is —O—CH₂—; and R¹¹ is pyrimidinyl, optionally substituted with 1 independently selected R^{w} groups; and R^{w}, at each occurrence, is independently-N(C₁-C₆ alkylenyl)₂-G⁴. In another embodiment of Formula (I), W is —O—CH₂—; and R¹¹ is pyrimidinyl, optionally substituted with 1 independently selected R^{w} groups; and R^{w}, at each occurrence, is independently-(C₁-C₆ alkylenyl)-G⁴. In another embodiment of Formula (I), W is —O—CH₂—; and R¹¹ is pyrimidinyl, optionally substituted with 1 independently selected R^{w} groups; and R^{w} is independently G⁴.

In one embodiment of Formula (I), R^{11a} and R^{11c}, at each occurrence, are each independently hydrogen, C₁-C₆ alkyl, C₂-C₆ alkenyl, or C₁-C₆ haloalkyl. In another embodiment of Formula (I), R^{11a} is C₁-C₆ alkyl or C₁-C₆ haloalkyl, —(C₂-C₆ alkylenyl)-OR^{11d}, (C₂-C₆ alkylenyl)-N(R^{11e})₂, or —(C₂-C₆ alkylenyl)-G⁴; and R^{11b}, at each occurrence, is independently C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11c})₂, or —(C₂-C₆ alkylenyl)-G⁴. In another embodiment of Formula (I), R^{11a} is C₁-C₆ alkyl or C₁-C₆ haloalkyl. In another embodiment of Formula (I), R^{11a} is C₁-C₆ alkyl or C₁-C₆ haloalkyl. In another embodiment of Formula (I), R^{11a} is —(C₂-C₆ alkylenyl)-G⁴.

In one embodiment of Formula (I), G⁴, at each occurrence, is independently R^{x1}, phenyl, monocyclic heteroaryl, C₃-C₁₁ cycloalkyl, C₄-C₁₁ cycloalkenyl, or 4-11 membered heterocycle; wherein each phenyl, monocyclic heteroaryl, C₃-C₁₁ cycloalkyl, C₄-C₁₁ cycloalkenyl, and 4-11 membered heterocycle is optionally substituted with 1, 2, 3, or 4 substituents independently selected from the group consisting of G⁵, R^{y}, —(C₁-C₆ alkylenyl)-G⁵, -L³-(C₁-C₆ alkylenyl)ₛ-R^{x1}, -L³-(C₃-C₇ cycloalkyl)-R^{x1}, -L³-(C₄-C₇ cycloalkenyl)-R^{x1}, -L³-(4-7 membered heterocycle)-R^{x1}, and -L²-(C₁-C₆ alkylenyl)ₛ-G⁵; and L¹ is O, C(O), N(H), N(C₁-C₆ alkyl), NHC(O), C(O)O, S, S(O), or S(O)₂; L³ is bond, O, C(O), N(H), N(C₁-C₆ alkyl), NHC(O), N(C₁-C₆ alkyl) C(O), N[(C₁-C₆ alkyl)ₛ-R^{x1}], N[(C₁-C₆ alkyl)ₛ-R^{x1}]C(O), S, S(O), or S(O)₂, C(O)NH, C(O)N(C₁-C₆ alkyl), or C(O)N[(C₁-C₆ alkyl)ₛ-R^{x1}]; and s is 0 or 1. In another embodiment of Formula (I), G⁴, at each occurrence, is independently R^{x1}, phenyl, monocyclic heteroaryl, C₃-C₁₁ cycloalkyl, C₄-C₁₁ cycloalkenyl, or 4-11 membered heterocycle; wherein each phenyl, monocyclic heteroaryl, C₃-C₁₁ cycloalkyl, C₄-C₁₁ cycloalkenyl, and 4-11 membered heterocycle is optionally substituted with 1, or 2 substituents independently selected from the group consisting of R^{y}, -L³-(C₁-C₆ alkylenyl)ₛ-R^{x1}, —(C₁-C₆ alkylenyl)ₛ-L³-(C₁-C₆ alkylenyl)ₛ-R^{x1}, and -L²-(C₁-C₆ alkylenyl)ₛ-G⁵; L¹ is O; L³ is bond, O, C(O), or C(O)NH; and s, at each occurrence, is independently is 0 or 1. In another embodiment of Formula (I), G⁴, at each occurrence, is independently 4-11 membered heterocycle; wherein each 4-11 membered heterocycle is optionally substituted with 1, or 2 substituents independently selected from the group consisting of R^{y}, -L³-(C₁-C₆ alkylenyl)ₛ-R^{x1}, —(C₁-C₆ alkylenyl)ₛ-L³-(C₁-C₆ alkylenyl)ₛ-R^{x1}, and -L²-(C₁-C₆ alkylenyl)ₛ-G⁵; L¹ is O, L³ is bond, O, C(O), or C(O)NH; and s, at each occurrence, is independently is 0 or 1. In another embodiment of Formula (I), G⁴, at each occurrence, is independently phenyl substituted with -L³-(C₁-C₆ alkylenyl)ₛ-R^{x1}; L³ is bond or O; and s is 0 or 1. In another embodiment of Formula (I), G⁴, at each occurrence, is independently phenyl optionally substituted with 1 —OCH₃.

In one embodiment of Formula (I), G⁵, at each occurrence, is independently phenyl, monocyclic heteroaryl, C₃-C₇ monocyclic cycloalkyl, C₄-C₇ monocyclic cycloalkenyl, or 4-12 membered heterocycle; wherein each G⁵ is optionally substituted with 1 independently selected R^{z} group. In another embodiment of Formula (I), G⁵, at each occurrence, is independently 4-12 membered heterocycle.

In one embodiment of Formula (I), R^{x1}, at each occurrence, is independently selected from the group consisting of a polyethylene glycol, a polyol, a polyether, CH₂P(O)(R^{k})₂, C(O)OH, S(O)(═NH)(C₁-C₃ alkyl), a carboxylic acid isostere, C₃-C₁₁ cycloalkyl, C₄-C₁₁ cycloalkenyl, or 4-11 membered heterocycle wherein the C₃-C₁₁ cycloalkyl, C₄-C₁₁ cycloalkenyl, and 4-11 membered heterocycle are substituted with two or more ORⁿ groups and optionally substituted with 1 independently selected R^{z} group,

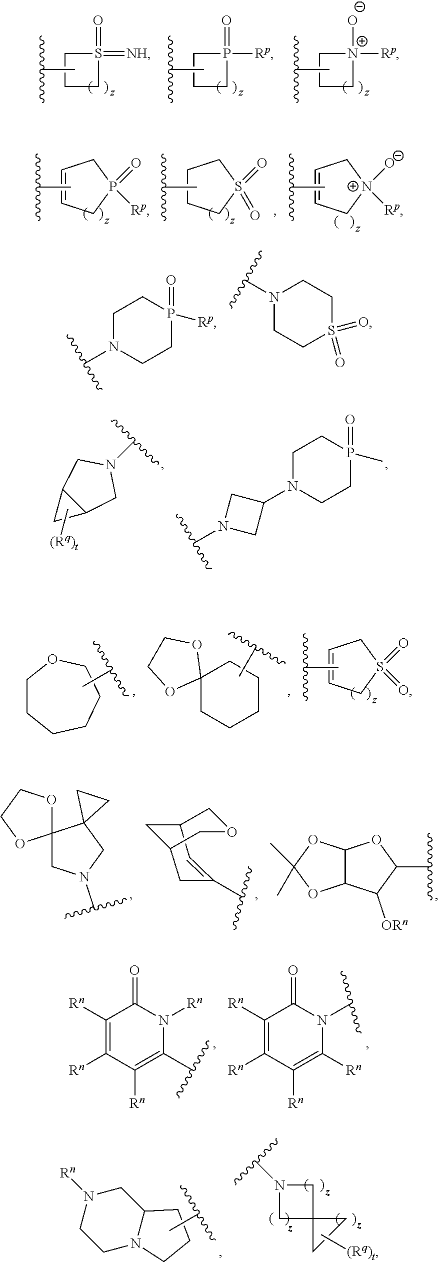

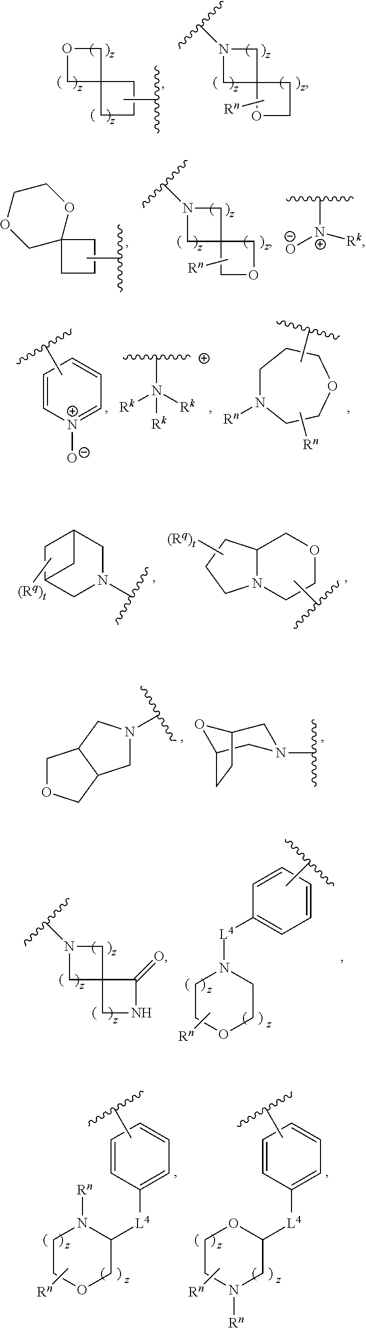

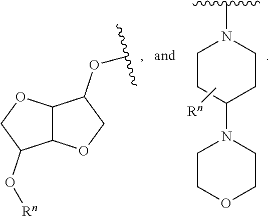

In another embodiment of Formula (I), R^{x1}, at each occurrence, is independently selected from the group consisting of a polyethylene glycol, a polyol, a polyether, CH₂P(O)(R^{k})₂, C(O)OH, S(O)(═NH)(C₁-C₃ alkyl), C₃-C₁₁ cycloalkyl, or 4-11 membered heterocycle wherein the C₃-C₁₁ cycloalkyl, and 4-11 membered heterocycle are substituted with two or more ORⁿ groups.

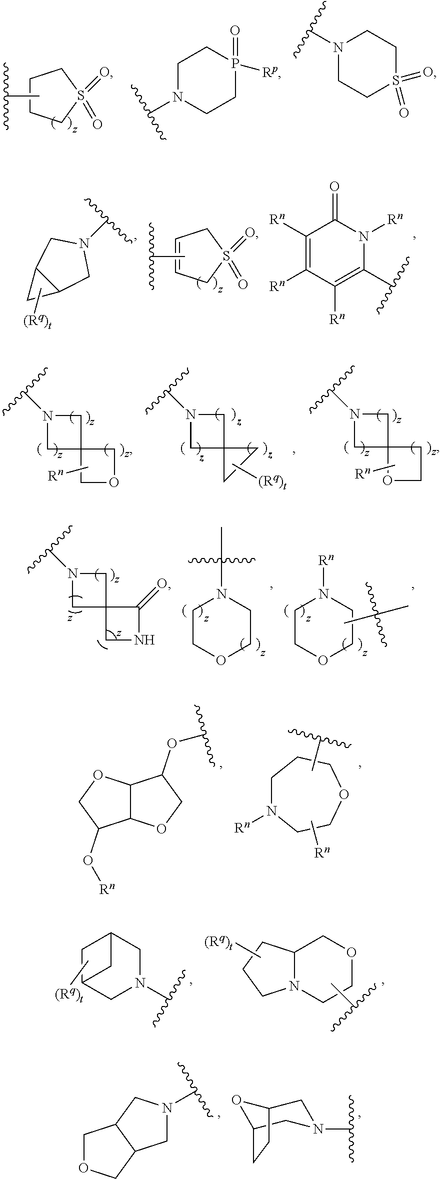

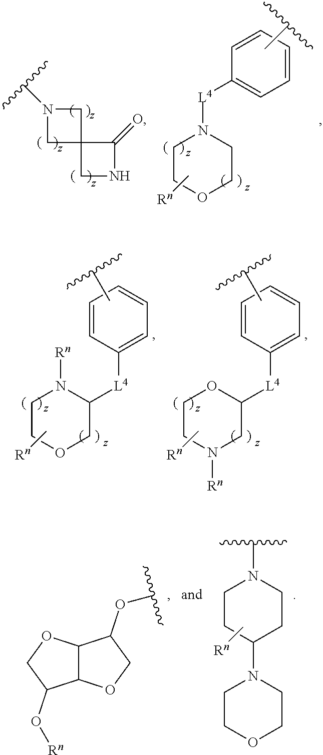

In another embodiment of Formula (I), R^{x1}, at each occurrence, is independently selected from the group consisting of a polyethylene glycol or 4-11 membered heterocycle wherein the 4-11 membered heterocycle is substituted with two or more ORⁿ groups.

In one embodiment of Formula (I), R^{x1}, at each occurrence, is polyethylene glycol. In another embodiment of Formula (I), R^{x1}, at each occurrence, is polyethylene glycol, selected from the group consisting of

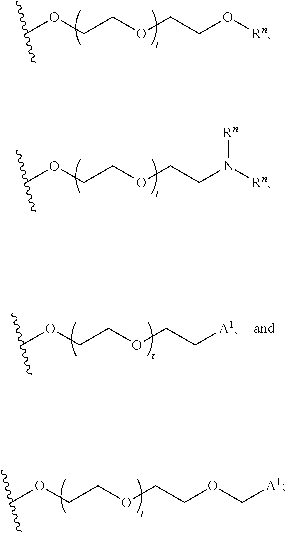

wherein t is an integer from 1-10; Rⁿ is hydrogen or C₁-C₆ alkyl; and A¹ is a 4-12 membered heterocyclyl optionally substituted with 1 independently selected R^{z} group. In another embodiment of Formula (I), R^{x1}, at each occurrence, is selected from the group consisting of

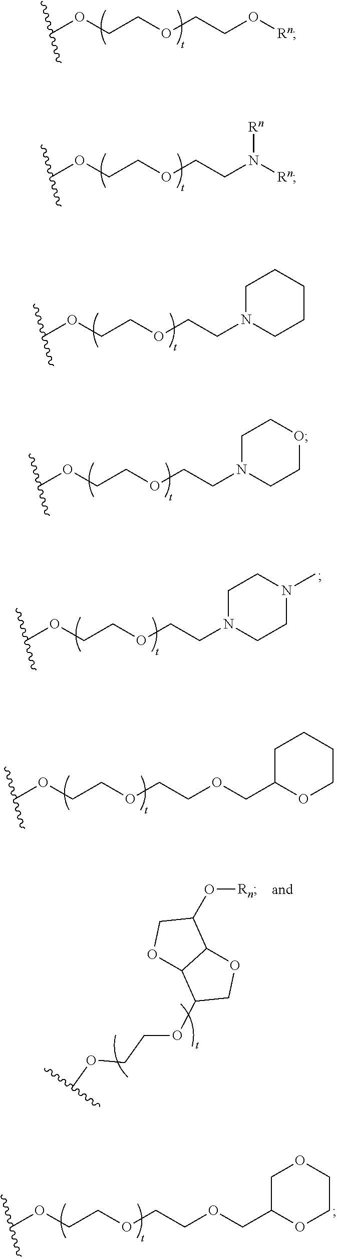

wherein t is an integer from 1-10 and Rⁿ is hydrogen or C₁-C₆ alkyl. In one embodiment of Formula (I), R^{x1}, at each occurrence, is polyethylene glycol. In another embodiment of Formula (I), R^{x1}, at each occurrence, is polyethylene glycol, selected from the group consisting of

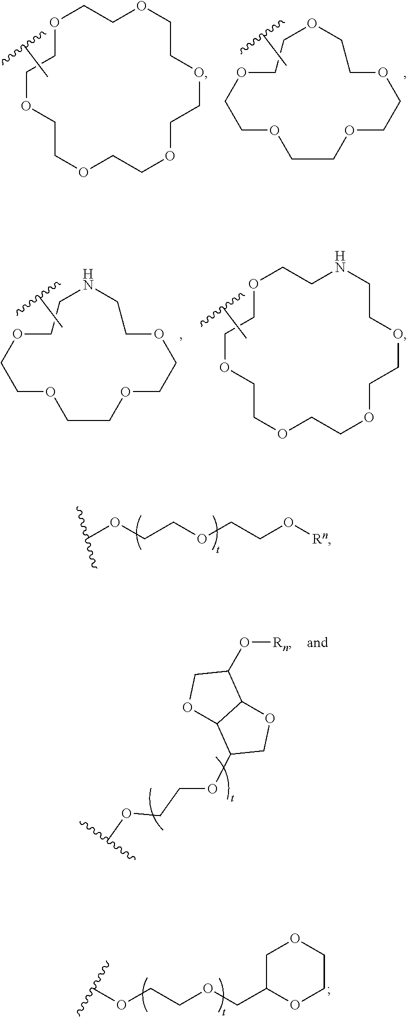

wherein t is an integer from 1-10; and Rⁿ is hydrogen or C₁-C₆ alkyl. In another embodiment of Formula (I), R^{x1}, at each occurrence, is a polyol or a polyether. In another embodiment of Formula (I), R^{x1}, at each occurrence, is a polyol or a polyether selected from the group consisting of

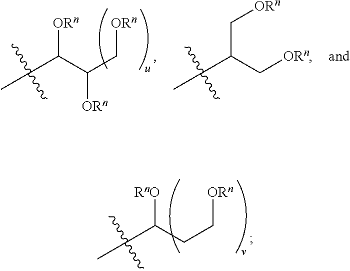

wherein Rⁿ is hydrogen or C₁-C₆ alkyl; u is an integer from zero to 4; and v is an integer from 1-2. In another embodiment of Formula (I), R^{x1}, at each occurrence, is selected from the group consisting of

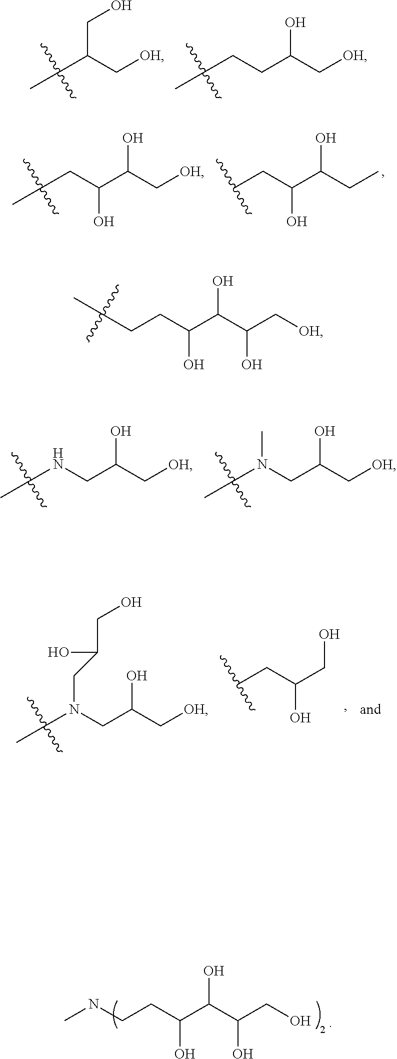

In another embodiment of Formula (I), R^{x1}, at each occurrence, is selected from the group consisting of

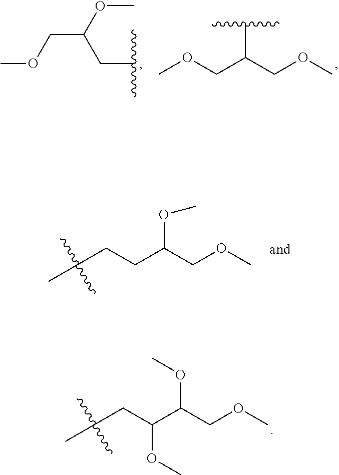

In another embodiment of Formula (I), R^{x1}, at each occurrence, is 4-11 membered heterocycle wherein the 4-11 membered heterocycle is substituted with two or more ORⁿ groups wherein Rⁿ is hydrogen or C₁-C₆ alkyl. In another embodiment of Formula (I), R^{x1}, at each occurrence, is C₃-C₁₁ cycloalkyl, C₄-C₁₁ cycloalkenyl, or 4-11 membered heterocycle wherein the C₃-C₁₁ cycloalkyl, C₄-C₁₁ cycloalkenyl, or 4-11 membered heterocycle are substituted with two or more ORⁿ groups; wherein Rⁿ is hydrogen or C₁-C₆ alkyl. In another embodiment of Formula (I), R^{x1}, at each occurrence, is selected from the group consisting of

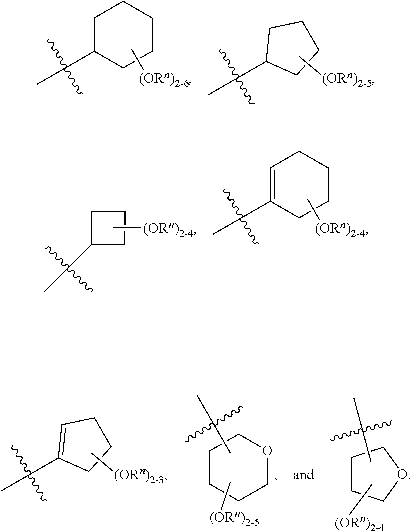

In one embodiment of Formula (I), L⁴ is C₁-C₆ alkyl, —O—C₁-C₆ alkyl, C₁-C₆ alkyl-O—, C(O), N(H), N(C₁-C₆ alkyl), NHC(O), OC(O), C(O)O, or S(O)₂. In another embodiment of Formula (I), L⁴ is CH₂, OCH₂, OCH₂CH₂, OC(O), or S(O)₂.

In one embodiment of Formula (I), R^{k}, at each occurrence, is independently C₁-C₆ alkyl or C₁-C₆ haloalkyl. In another embodiment of Formula (I), R^{k}, at each occurrence, is independently C₁-C₆ alkyl.

In one embodiment of Formula (I), Rⁿ, at each occurrence, is independently hydrogen, or C₁-C₆ alkyl.

In one embodiment of Formula (I), R^{p} is C₁-C₃ alkyl, or cyclopropyl. In another embodiment of Formula (I), R^{p} is C₁-C₃ alkyl.

In one embodiment of Formula (I), R^{q}, at each occurrence, is independently C(O)OH, —OH, halogen, —O—C₁-C₆ alkyl, or C₁-C₆ alkyl. In another embodiment of Formula (I), C(O)OH, —OH, halogen, or —O—C₁-C₆ alkyl.

In one embodiment of Formula (I), t is 0, 1, or 2.

In one embodiment of Formula (I), z, at each occurrence, is independently 1, 2, 3, or 4. In another embodiment of Formula (I),), z, at each occurrence, is independently 1, 2, or 34.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen; and
R⁷, R¹² and R¹⁶ are each independently hydrogen.

In one embodiment of Formula (I),
A² is N;
A³ is C;
A⁴ is O;
A⁶ is C;
R^{A} is hydrogen;
X is O;
R⁹ is —OH;
R^{10A} and R^{10B}, are each independently hydrogen; and
R⁷, R¹² and R¹⁶ are each independently hydrogen.

In one embodiment of Formula (I),
A² is N;
A³ is C;
A⁴ is S;
A⁶ is C;
R^{A} is hydrogen;
X is O,
R′ is —OH;
R^{10A} and R^{10B}, are each independently hydrogen; and
R⁷, R¹² and R¹⁶ are each independently hydrogen.

In one embodiment of Formula (I),
A² is N;
A³ is C;
A⁴ is S;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya}); and
m is 3.

In one embodiment of Formula (I),
A² is N;
A³ is C;
A⁴ is S;
A⁶ is C;
R^{A} is hydrogen;
X is O,
R⁹ is —OH;
R^{10A} and R^{10B}, are each independently hydrogen;
R⁷, R¹² and R¹⁶ are each independently hydrogen;
Y is (CH₂)ₘ; wherein 2 CH₂ groups are each independently replaced by O and 1 CH₂ group is replaced by C(R^{ya})(R^{yb}); and
m is 4.

In one embodiment of Formula (I),
A² is CH;
A³ is N;
A⁴ is CH;
A⁶ is C;
R^{A} is hydrogen;
X is O,
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya});
m is 3; and
G¹ is piperazinyl substituted with 1 R^{s}.

In one embodiment of Formula (I),
A² is CH;
A³ is N;
A⁴ is CH;
A⁶ is C;
R^{A} is hydrogen;
X is O,
R⁹ is —OH;
R^{10A} and R^{10B}, are each independently hydrogen;
R⁷, R¹² and R¹⁶ are each independently hydrogen;
Y is (CH₂)ₘ; wherein 2 CH₂ groups are each independently replaced by O and 1 CH₂ group is replaced by C(R^{ya})(R^{yb});
m is 4; and
G¹ is piperazinyl substituted with 1 R^{s}.

In one embodiment of Formula (I),
A² is CH;
A³ is N;
A⁴ is CH;
A⁶ is C;
R^{A} is hydrogen;
X is O,
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya});
m is 3;
G¹ is piperazinyl substituted with 1 R^{s};
W is -L¹-CH₂; and
L¹ is independently O.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 2 CH₂ groups are each independently replaced by O and 1 CH₂ group is replaced by C(R^{ya})(R^{yb});

m is 4;
G¹ is piperazinyl substituted with 1 R^{s};
W is -L¹-CH₂; and
L¹ is independently O.

In one embodiment of Formula (I),
A² is CH;
A³ is N;
A⁴ is CH;
A⁶ is C;
R^{A} is hydrogen;
X is O,
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya});
m is 3;
G¹ is piperazinyl substituted with 1 R^{s};
W is -L¹-CH₂;
L¹ is independently O;
W is —O—CH₂—, and
   R¹¹ is pyrimidinyl, optionally substituted with 1, 2, or 3 independently selected R^{w} groups. One embodiment pertains to compounds of Formula (I), or pharmaceutically acceptable salts thereof,
      wherein
G⁴, at each occurrence, is independently phenyl substituted with 1 -L³-(C₁-C₆ alkylenyl)ₛ-R^{x1};
L³ is bond or O;
s, at each occurrence, is independently is 0 or 1;
R^{x1}, at each occurrence, is independently selected from the group consisting of a polyethylene glycol, or 4-11 membered heterocycle wherein the 4-11 membered heterocycle is substituted with two or more ORⁿ groups; and
Rⁿ is hydrogen or C₁-C₆ alkyl.

One embodiment pertains to compounds of Formula (I), or pharmaceutically acceptable salts thereof,

wherein
A² is N, A³ is C, A⁴ is S and A⁶ is C;
R^{A} is hydrogen;
X is O;
Y is (CH₂)ₘ; wherein 1 or 3 CH₂ groups are each independently replaced by O, N(R^{ya}), or C(R^{ya})(R^{yb});
m is 3 or 4;
R^{ya}, at each occurrence, is independently hydrogen or C₁-C₆ alkyl; wherein the C₁-C₆ alkyl is optionally substituted with 1 G¹; and
R^{yb} is C₁-C₆ alkyl; wherein the C₁-C₆ alkyl is optionally substituted with 1 G¹;
G¹, at each occurrence, is a 4-11 membered heterocycle; wherein each G¹ is optionally substituted with 1, 2, or 3 substituents independently selected from the group consisting of -L^{1A}-(C₁-C₆ alkylenyl)ₛ-R^{x1}, and R^{s};
L^{1A} is bond;
R⁵ is independently G³;
G³, at each occurrence, is independently C₆-C₁₀ aryl; wherein each G³ is optionally substituted with 1, 2, or 3 R^{v} groups;
A⁷ is CR⁷;
A⁸ is CR⁸;
A¹⁵ is CR¹⁵;
R⁷, R¹² and R¹⁶ are each independently hydrogen;
R⁸, R¹³, R¹⁴, and R¹⁵, are each independently hydrogen, halogen, or C₁-C₄ alkyl; or
R⁹ is —OH;
R^{10A} and R^{10B}, are each independently hydrogen;
W is -L¹-CH₂;
R¹¹ is a 5-11 membered heteroaryl; wherein each R¹¹ is optionally substituted with 1, 2, or 3 independently selected R^{w} groups;
R^{w}, at each occurrence, is independently G⁴;
G⁴, at each occurrence, is independently phenyl; wherein each G₄ is optionally substituted with 1, 2, 3, or 4 substituents independently selected from the group consisting of R^{y}, and -L³-(C₁-C₆ alkylenyl)ₛ-R^{x1};
L³ is bond, or O;
s, at each occurrence, is independently is 0 or 1;
R^{s}, and R^{y}, at each occurrence, are each independently C₁-C₆ alkyl, or —OR^{m}, —R^{m} is C₁-C₆ alkyl;
R^{x1}, at each occurrence, is independently selected from the group consisting of a polyethylene glycol, and 4-11 membered heterocycle wherein the 4-11 membered heterocycle is substituted with two or more ORⁿ; and
Rⁿ is hydrogen or C₁-C₆ alkyl;
wherein at least one R^{x1} is present.

One embodiment pertains to compounds of Formula (I), or pharmaceutically acceptable salts thereof,

wherein
A² is N, A³ is C, A⁴ is O or S and A⁶ is C;
R^{A} is hydrogen;
X is O;
Y is (CH₂)ₘ; wherein 1, 2, or 3 CH₂ groups are each independently replaced by O, N(R^{ya}), or C(R^{ya})(R^{yb});
m is 3 or 4;
R^{ya}, at each occurrence, is independently hydrogen, or C₁-C₆ alkyl; wherein the C₁-C₆ alkyl is optionally substituted with G¹;
R^{yb} is C₁-C₆ alkyl; wherein the C₁-C₆ alkyl is optionally substituted with G¹;
G¹, at each occurrence, is a 4-11 membered heterocycle; wherein each G¹ is optionally substituted with 1 substituent independently selected from the group consisting of L^{1A}-(C₁-C₆ alkylenyl)ₛ-R^{x1} and R^{s};
L^{1A} is bond;
R⁵ is independently G³;
G³, at each occurrence, is independently C₆-C₁₀ aryl, 5-11 membered heteroaryl, C₃-C₁₁ cycloalkyl, C₄-C₁ cycloalkenyl, or 4-7 membered heterocycle; wherein each G³ is optionally substituted with 1 R^{v} group;
A⁷ is N or CR⁷;
A⁸ is N or CR⁸;
A¹⁵ is N or CR¹⁵;
R⁷, R¹² and R¹⁶ are each independently hydrogen;
R⁸, R¹³, R¹⁴, and R¹⁵, are each independently hydrogen, halogen, or C₁-C₄ alkyl;
R⁹ is —OH;
R^{10A} and R^{10B} are each independently hydrogen;
W is -L¹-CH₂; wherein L¹ at each occurrence, is independently O;
R¹¹ is a C₆-C₁₀ aryl or a 5-11 membered heteroaryl; wherein each R¹¹ is optionally substituted with 1 or 2 independently selected R^{w} groups;
R^{w}, at each occurrence, is independently —OR^{11a}, G⁴, N(C₁-C₆ alkylenyl)₂-G⁴, or (C₁-C₆ alkylenyl)-G⁴;
R^{11a}, at each occurrence, is independently G⁴ or —(C₂-C₆ alkylenyl)-G⁴;
G⁴, at each occurrence, is independently R^{x1}, phenyl, monocyclic heteroaryl, C₃-C₁₁ cycloalkyl, C₄-C₁₁ cycloalkenyl, or 4-11 membered heterocycle; wherein each phenyl, monocyclic heteroaryl, C₃-C₁ cycloalkyl, C₄-C₁ cycloalkenyl, and 4-11 membered heterocycle is optionally substituted with 1 or 2 substituents independently selected from the group consisting of R^{y}, -L³-(C₁-C₆ alkylenyl), —R^{x1}, (C₁-C₆ alkylenyl), -L³-(C₁-C₆ alkylenyl)ₛ-R^{x1}, and -L²-(C₁-C₆ alkylenyl)ₛ-G⁵;
L² is O,
L³ is bond, O, C(O), or C(O)NH;
s, at each occurrence, is independently is 0 or 1;
G⁵, at each occurrence, is independently 4-12 membered heterocycle;
R^{s}, R^{v}, and R^{y}, at each occurrence, are each independently C₁-C₆ alkyl, halogen, or —OR^{m};
R^{m} is C₁-C₆ alkyl;
R^{x1}, at each occurrence, is independently selected from the group consisting of a polyethylene glycol, polyol, polyether, CH₂P(O)(R^{k})₂, C(O)OH, S(O)(═NH)(C₁-C₃ alkyl), C₃-C₁₁ cycloalkyl, or 4-11 membered heterocycle wherein the C₃-C₁ cycloalkyl and 4-11 membered heterocycle are substituted with two or more ORⁿ groups and optionally substituted with 1 independently selected R^{z} group,

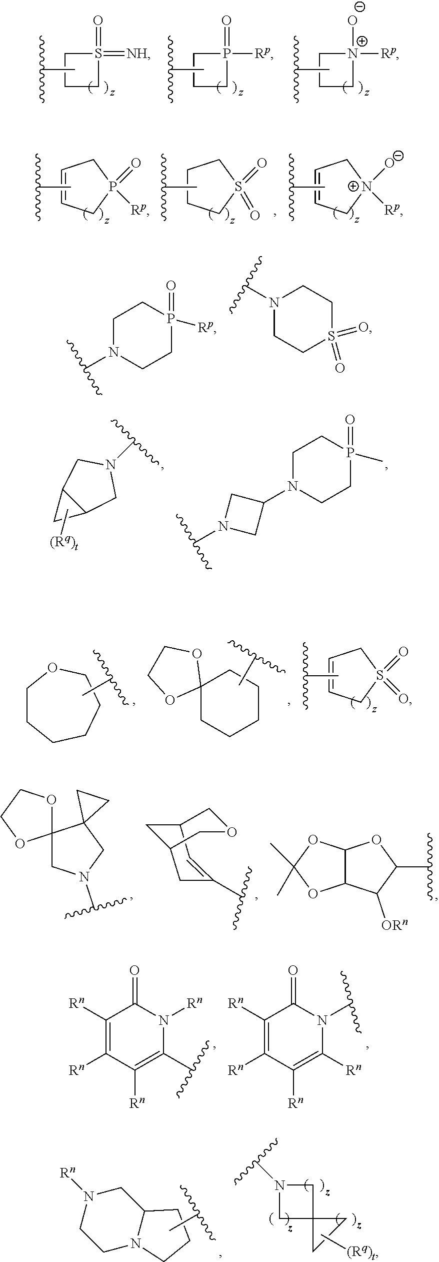

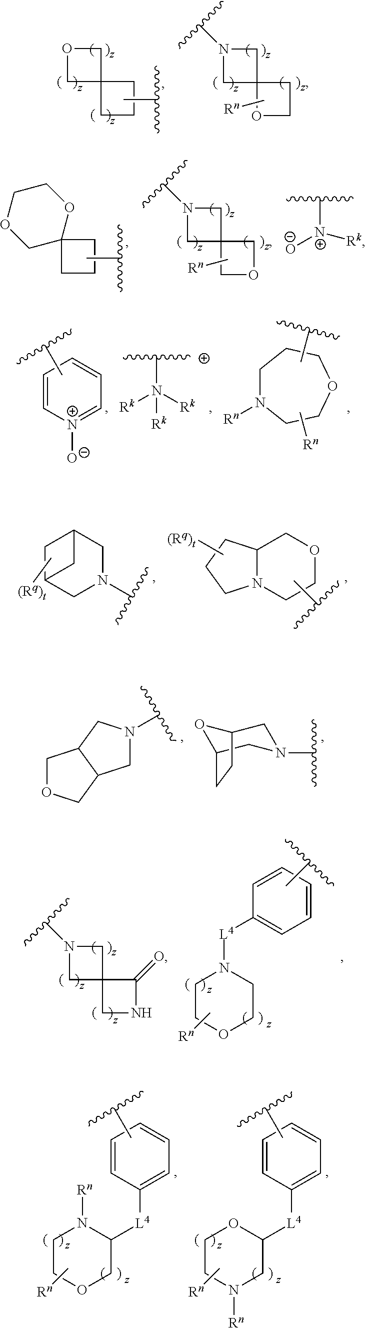

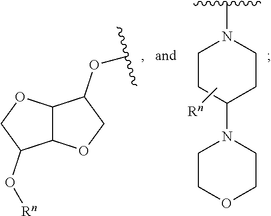

L⁴ is C₁-C₆ alkyl, —O—C₁-C₆ alkyl, OC(O), or S(O)₂;
R^{k}, at each occurrence, is independently C₁-C₆ alkyl;
Rⁿ, at each occurrence, is independently hydrogen, C₁-C₆ alkyl, or C₁-C₆ alkyl;
R^{p} is C₁-C₃ alkyl;
R^{q}, at each occurrence, is independently C(O)OH, halogen, or —O—C₁-C₆ alkyl;
t is 0, 1, or 2; and
z, at each occurrence, is independently 1, 2, or 3;
wherein at least one R^{x1} is present.

Exemplary compounds of Formula (I) include, but are not limited to:
(7R,16R,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7S,16R,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R,21R)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-{[2-(2-methoxyphenyl)pyrimidin-4-yl]methoxy}-20-methyl-16-{[4-(2,5,8,11-tetraoxatridecan-13-yl) piperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-16-[(4-{2-[2-(2-methoxyethoxy)ethoxy]ethyl}piperazin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
methyl 6-(4-{[(7R,16R,21S)-7-carboxy-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]inden-16-yl]methyl}piperazin-1-yl)-6-deoxy-2,3,4-tri-O-methyl-a-D-mannopyranoside;
methyl 6-O-{3-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside;
methyl 6-O-{3-[4-({[(7S,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside;
methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-glucopyranoside;
methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-mannopyranoside;
methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-2,3,4-tri-O-methyl-a-D-mannopyranoside;
methyl 6-O-{4-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-a-D-mannopyranoside;
(7R,16R,21S)-19-chloro-1-(4-fluorophenyl)-10-{[2-(4-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
methyl 6-O-{4-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside;
(7R,16R,21S)-19,23-dichloro-1-(4-fluorophenyl)-10-{[2-(2-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-10-({2-[4-(2-carboxyethyl)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-10-[(2-{4-[(2R)-2,3-dihydroxypropoxy]phenyl}pyrimidin-4-yl)methoxy]-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-{[2-(2-carboxyphenyl)pyrimidin-4-yl]methoxy}-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[4-(2-carboxyethyl)phenyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-16-[(4-{2-[2-(2-methoxyethoxy)ethoxy]ethyl}piperidin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{4-[(2R)-2,3-dihydroxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{2-[(2R)-2,3-dihydroxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-10-({2-[2-(carboxymethoxy)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-{[2-(4-methyl-4-oxo-1,4λ⁵-azaphosphinan-1-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[2-(S-methanesulfonimidoyl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-16-[(4-{2-[2-(2-methoxyethoxy)ethoxy]ethyl}piperidin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19-chloro-1-(4-fluorophenyl)-20-methyl-10-{[2-(1-methyl-6-oxo-1,6-dihydropyridin-2-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclopropyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[4-(S-methanesulfonimidoyl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[(1s,4s)-4-(carboxymethyl)cyclohexyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[(1r,4r)-4-(carboxymethyl)cyclohexyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-{[2-(6,6-difluoro-3-azabicyclo[3.1.0]hexan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-{[2-(1,1-dioxo-1,2,3,6-tetrahydro-1λ⁶-thiopyran-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[(4S*)-4-(carboxymethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[(1R,5S,6r)-6-carboxy-3-azabicyclo[3.1.0]hexan-3-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[(4R*)-4-(carboxymethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(1S,2S)-1,2-dihydroxycyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-({2-[(1R,2R)-1,2-dihydroxycyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-{[2-(1,1-dioxo-1λ⁶-thian-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[4-(carboxymethyl)-4-methylpiperidin-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methyl piperazin-1-yl)methyl]-10-{[2-(2-oxa-6-azaspiro[3.3]heptan-6-yl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19-chloro-1-(4-fluorophenyl)-20-methyl-16-[(4-methylpiperazin-1-yl)methyl]-10-({2-[(2R)-1-(23-oxo-2,5,8,11,14,17,20-heptaoxatricosan-23-yl) pyrrolidin-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[1-(2,5,8,11,14,17,20,23,26,29,32,35,38-tridecaoxanonatriacontan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(6-{2-[2-(2-methoxyethoxy)ethoxy]ethoxy}pyridin-3-yl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[(2,5,8,11,14,17,20,23,26,29,32-undecaoxatetratriacontan-34-yl) carbamoyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-{[2-(6,6-difluoro-2-azaspiro[3.3]heptan-2-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-10-({2-[4-(carboxymethyl)piperidin-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methyl piperazin-1-yl)methyl]-10-({2-[4-(35-oxo-2,5,8,11,14,17,20,23,26,29,32-undecaoxa-36-azaheptatriacontan-37-yl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{3-[(dimethylphosphoryl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(4-{2-[2-(2-methoxyethoxy)ethoxy]ethyl}piperidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[4-(2,5,8,11-tetraoxatetradecan-14-yl) piperazin-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(2-{4-[(2,5,8,11,14,17,20-heptaoxadocosan-22-yl)oxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19-chloro-10-[(2-{(2R)-1-[3-(dimethylphosphoryl)propanoyl]pyrrolidin-2-yl}pyrimidin-4-yl)methoxy]-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(3S,4S)-3,4-dihydroxypyrrolidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[1-(2,5,8,11,14-pentaoxapentadecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[1-(2,5,8,11,14,17-hexaoxaoctadecan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[1-(2,5,8,11,14,17,20-heptaoxahenicosan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-{[2-(1-oxo-2,9-diazaspiro[5.5]undecan-9-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-{[2-(1,3-dihydroxypropan-2-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{1-[(2,5,8,11,14-pentaoxahexadecan-16-yl)oxy]cyclobutyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{1-[(2,5,8,11-tetraoxatridecan-13-yl)oxy]cyclobutyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-({2-[3-(1,3-dihydroxypropan-2-yl)azetidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(2-{(1r,4r)-4-[(2,5,8,11,14,17-hexaoxanonadecan-19-yl)oxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[(2S)-2-(2,5,8,11-tetraoxadodecan-1-yl) morpholin-4-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11-tetraoxatridecan-13-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid:
(7R,16R)-19,23-dichloro-1-(4-fluorophenyl)-10-({2-[(1s,4s)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-[(2-{4-[2-(4-methyl-4-oxo-1, 4λ⁵-azaphosphinan-1-yl)ethoxy]phenyl}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-{[2-(1-{[2-(2-{[(2R)-1,4-dioxan-2-yl]methoxy}ethoxy)ethoxy]methyl}cyclobutyl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-{[2-(1,1-dioxo-1λ⁶-thiolan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(2-{1-[(2,5,8,11,14,17-hexaoxanonadecan-19-yl)oxy]cyclopentyl}pyrimidin-4-yl)methoxy]-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-{[2-(1,1-dioxo-1λ⁶-thiomorpholin-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[1-(2,5,8,11,14,17-hexaoxaoctadecan-1-yl)cyclopentyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{1-[(2-{[(2R)-1,4-dioxan-2-yl]methoxy}ethoxy)methyl]cyclobutyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19-chloro-10-{[2-(3,3-difluoro-1-oxa-8-azaspiro[4.5]decan-8-yl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(6-methoxy-2-azaspiro[3.3]heptan-2-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-({2-[1-(2-{[(2R)-1,4-dioxan-2-yl]methoxy}ethoxy)-2-methylpropan-2-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{1-[(2-{[(2R)-1,4-dioxan-2-yl]methoxy}ethoxy)methyl]cyclopentyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{(1s,4s)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(2R)-2-(2,5,8,11,14,17-hexaoxaoctadecan-1-yl) morpholin-4-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(4R*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(4S*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(4S*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(4R*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[1-(2,5,8,11,14,17-hexaoxaoctadecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[(2,5,8,11-tetraoxatridecan-13-yl)oxy]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-cyclohexyl-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[4-({2-[(1,4-dioxan-2-yl)methoxy]ethoxy}methyl)-4-fluoropiperidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(1r,4r)-4-{2-[(1,4-dioxan-2-yl)methoxy]ethoxy}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-{[2-(bis{2-[2-(2-methoxyethoxy)ethoxy]ethyl}amino)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[3-(2,5,8,11-tetraoxadodecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[3-(2,5,8,11,14-pentaoxapentadecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-({2-[(1s,4s)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(4S*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(1r,4r)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(6-{2-[2-(2-methoxyethoxy)ethoxy]ethoxy}pyridin-2-yl)methoxy]-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-({2-[(4R*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{1-[(2-{[(2R)-1,4-dioxan-2-yl]methoxy}ethoxy)methyl]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(2S)-2-({2-[(1,4-dioxan-2-yl)methoxy]ethoxy}methyl) morpholin-4-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(3-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(1r,4r)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(4R*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[(1,4,7,10,13-pentaoxacyclopentadecan-2-yl)methoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-[(2-{bis[2-(2-methoxyethoxy)ethyl]amino}pyrimidin-4-yl)methoxy]-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-({2-[bis(2,5,8,11-tetraoxatridecan-13-yl)amino]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{4-[(1,3-dimethoxypropan-2-yl)oxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(4R*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[(4R*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(1s,4s)-4-fluoro-4-(2,5,8,11,14,17-hexaoxaoctadecan-1-yl)cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(4S*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-{[2-(1,4,7,10-tetraoxa-13-azacyclopentadecan-13-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-{[2-(1,4,7,10,13-pentaoxa-16-azacyclooctadecan-16-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{3-[(1,1-dioxo-1λ⁶-thiomorpholin-4-yl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(1s,4s)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[(4S*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[(1R*,2R*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{(1r,4r)-4-[(1,4-dioxan-2-yl)methoxy]-1-[2-(2-methoxyethoxy)ethoxy]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-[(2-{(1s,4s)-4-[(1,4-dioxan-2-yl)methoxy]-1-[2-(2-methoxyethoxy)ethoxy]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(2-{4-[(1,4,7,10,13,16-hexaoxacyclooctadecan-2-yl)methoxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[(1S*,2S*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(cyclopent-1-en-1-yl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{4-[(1,1-dioxo-1λ⁶-thiomorpholin-4-yl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(4R)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(4S)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[(1,4,7,10-tetraoxa-13-azacyclopentadecan-13-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid; (7R,16R)-19,23-dichloro-1-cyclobutyl-10-({2-[(4R*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid; (7R,16R)-19,23-dichloro-1-cyclobutyl-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-cyclobutyl-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,16,17-tetrahydro-15H-18,21-etheno-13,9-(metheno)-6,14-dioxa-2-thia-3,5-diazacyclononadeca[1,2,3-cd]indene-7-carboxylic acid;
(7R,16R)-19,23-dichloro-1-(4-fluorophenyl)-10-({2-[2-(2-{[(3R,3aR,6R,6aR)-6-methoxyhexahydrofuro[3,2-b]furan-3-yl]oxy}ethoxy)ethoxy]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(2-{[(3R,3aR,6R,6aR)-6-methoxyhexahydrofuro[3,2-b]furan-3-yl]oxy}ethoxy)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(cyclopent-1-en-1-yl)-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-[(2-{4-[(2S)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-[(2-{4-[(2R)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(2-{(1r,4r)-4-[2-(2-{[(3R,3aR,6R,6aR)-6-methoxyhexahydrofuro[3,2-b]furan-3-yl]oxy}ethoxy)ethoxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(3-{[2-(2-methoxyethoxy)ethoxy]methyl}azetidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7S,16R)-19,23-dichloro-1-(4-fluorophenyl)-10-{[2-(3-{[2-(2-methoxyethoxy)ethoxy]methyl}azetidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-10-({2-[(1,3-dimethoxypropan-2-yl)oxy]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[2-(morpholin-4-yl)ethyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methyl piperazin-1-yl)methyl]-10-[(2-{3-[2-(morpholin-4-yl)ethyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-({2-[4-methyl-4-(morpholin-4-yl)piperidin-1-yl]pyrimidin-4-yl}methoxy)-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[4-(morpholine-4-sulfonyl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(2-{[(3R,3aR,6R,6aR)-6-methoxyhexahydrofuro[3,2-b]furan-3-yl]oxy}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{3-[(morpholin-4-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[(morpholin-4-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-({2-[3-(morpholine-4-sulfonyl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(3S,8aS)-hexahydro-1H-pyrrolo[2,1-c][1,4]oxazin-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[(morpholine-4-carbonyl)oxy]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-10-({2-[3,4-bis(2,5,8,11-tetraoxadodecan-1-yl)phenyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[2-{2-[2-(2-methoxyethoxy)ethoxy]ethoxy}-4-(2,5,8,11-tetraoxadodecan-1-yl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[4-(2-{[(3R,3aR,6R,6aR)-6-methoxyhexahydrofuro[3,2-b]furan-3-yl]oxy}ethoxy)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[4-{[(2R)-1,4-dioxan-2-yl]methoxy}-2-(2,5,8,11-tetraoxadodecan-1-yl)phenyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(3-{2-[2-(2-methoxyethoxy)ethoxy]ethoxy}phenyl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-{[2-(4-{[(2S)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-{[2-(4-{[(2R)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(2-{4-[2-(2-{[(3R,3aR,6R,6aR)-6-methoxyhexahydrofuro[3,2-b]furan-3-yl]oxy}ethoxy)ethoxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(1r,4r)-4-{[2-(2-methoxyethoxy)ethoxy]methyl}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(4-{[(3S,3aR,6R,6aR)-6-hydroxyhexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(5-{2-[2-(2-methoxyethoxy)ethoxy]ethoxy}pyridin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-{[2-(4-{[(3R)-4-methylmorpholin-3-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-{[2-(4-{2-[(3aR,6aS)-tetrahydro-1H-furo[3,4-c]pyrrol-5 (3H)-yl]ethoxy}phenyl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[2-(2-oxa-6-azaspiro[3.3]heptan-6-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[2-(tetrahydro-1H-furo[3,4-c]pyrrol-5 (3H)-yl)ethyl]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({2-[(1R,5S,6r)-6-hydroxy-3-azabicyclo[3,1,1]heptan-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[2-(morpholin-4-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({6-[2-(2-methoxyethoxy)ethoxy]-2-(2-methoxyphenyl)pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-16-[(4-methylpiperazin-1-yl)methyl]-10-[(2-{4-[2-(8-oxa-3-azabicyclo[3,2,1]octan-3-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-10-{[2-(3-azabicyclo[3,1,1]heptan-3-yl)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-10-({2-[(1R,5S)-6,6-difluoro-3-azabicyclo[3,1,1]heptan-3-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-{[2-(4-{[(2S)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(4-{[(3S,3aR,6S,6aR)-6-hydroxyhexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-{[2-(4-{[(2R)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-[(6-{2-[2-(2-methoxyethoxy)ethoxy]ethoxy}pyrazin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-20,22-dimethyl-10-[(2-{[(2S)-4-methyl-1,4-oxazepan-2-yl]methoxy}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-({3-[2-(2-methoxyethoxy)ethoxy]-6-(2-methoxyphenyl)pyridin-2-yl}methoxy)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid; and pharmaceutically acceptable salts thereof.

### Formula (II)

One embodiment pertains to compounds of Formula (IIa), (IIb), (IIc), (IId), or pharmaceutically acceptable salts thereof,

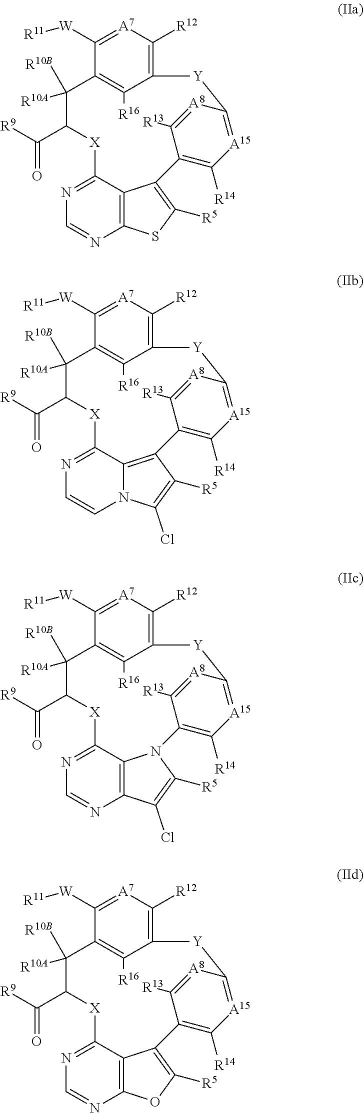

wherein A⁷, A⁸, A¹⁵, R⁵, R⁹, R^{10A}, R^{10B}, R¹¹, R¹², R¹³, R¹⁴, R¹⁶, W, X, and Y are as described in embodiments of Formula (I) herein.

Exemplary compounds of Formula Formula (IIa), (IIb), (IIc), and (IId) include, but are not limited to: Examples 1-178 and pharmaceutically acceptable salts thereof.

### Formula (III)

One embodiment pertains to compounds of Formula (IIIa), (IIIb), (IIIc), (IIId), or pharmaceutically acceptable salts thereof.

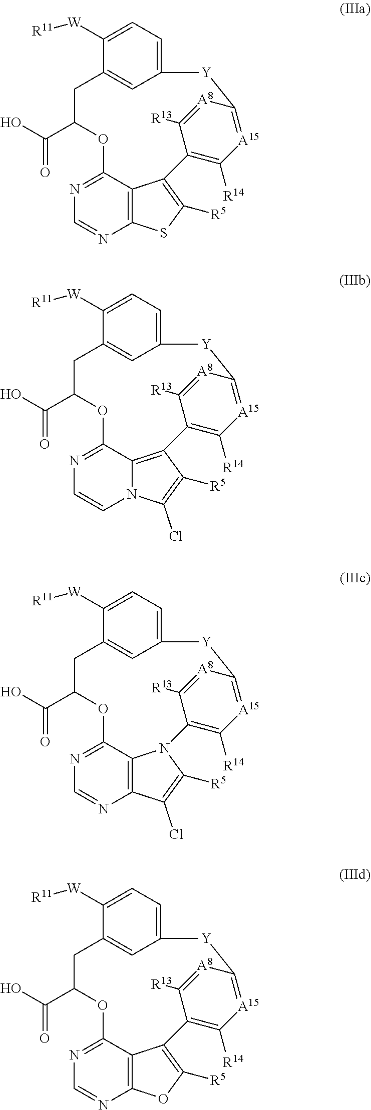

wherein A⁸, A¹⁵, R⁵, R¹¹, R¹³, R¹⁴, W, and Y are as described in embodiments of Formula (I) herein.

Exemplary compounds of Formula (IIIa), (IIIb), (IIIc), and (IIId) include, but are not limited to: Examples 1-178 and pharmaceutically acceptable salts thereof.

### Formula (IV)

One embodiment pertains to compounds of Formula (IVa), (IVb), (IVc), (IVd), or pharmaceutically acceptable salts thereof,

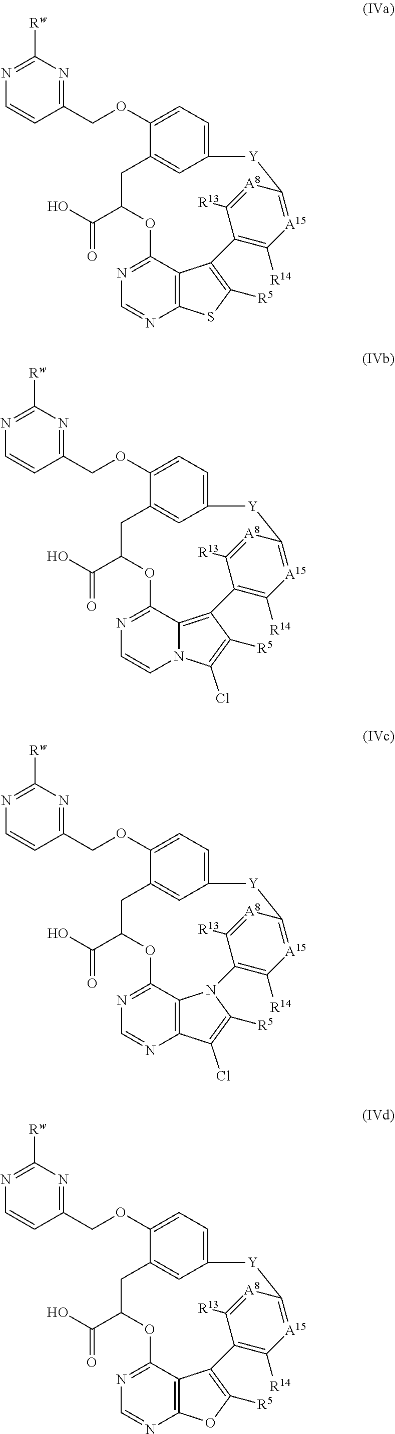

wherein A⁸, A¹⁵, R⁵, R¹³, R¹⁴, R^{w}, and Y are as described in embodiments of Formula (I) herein.

Exemplary compounds of Formula (IVa), (IVb), (IVc), and (IVd) include but are not limited to: Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146,147, 148, 149, 150, 151, 152, 153, 154, 155, 157, 158, 159, 160, 161, 163, 164, 165, 166, 167, 168, 169, 170, 171, 172, 173, 174, 175, 177, and pharmaceutically acceptable salts thereof.

### Formula (V)

One embodiment pertains to compounds of Formula (Va), (Vb), (Vc), (Vd), or pharmaceutically acceptable salts thereof,

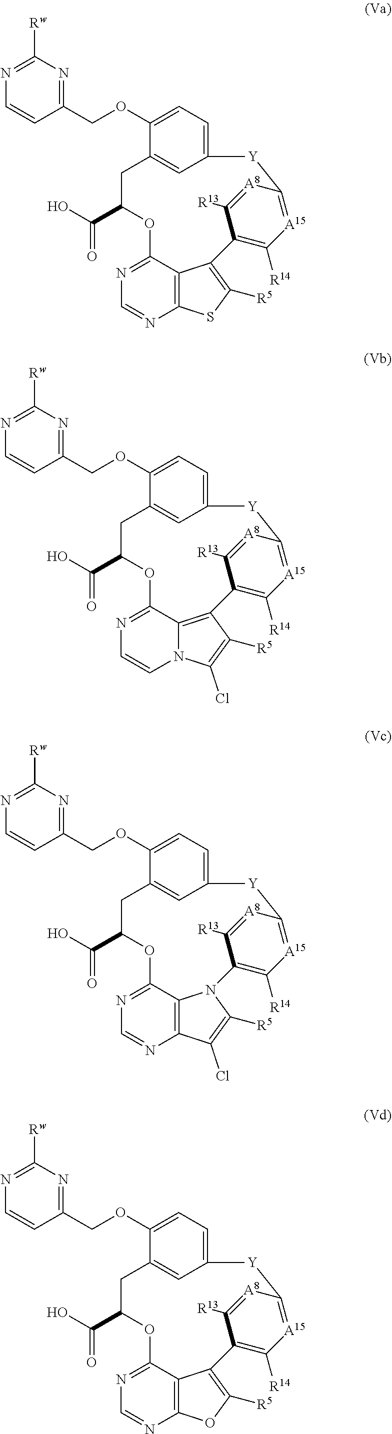

wherein A⁸, A¹⁵, R⁵, R¹³, R¹⁴, R^{w}, and Y are as described in embodiments of Formula (I) herein.

Exemplary compounds of Formula (Va), (Vb), (Vc), and (Vd) include but are not limited to: Examples 1, 2, 4, 5, 6, 7, 8, 9, 10, 11, 12, 13, 14, 17, 18, 19, 21, 23, 24, 28, and pharmaceutically acceptable salts thereof.

Compound names are assigned by using Name 2016.1.1 (File Version N30E41, Build 86668) or Name 2017.2.1 (File Version N40E41, Build 96719) naming algorithm by Advanced Chemical Development or Struct=Name naming algorithm as part of CHEMDRAW® ULTRA v. 12.0.2.1076 or Professional Version 15.0.0.106.

Compounds of the present disclosure may exist as atropisomers, resulting from hindered rotation about a single bond, when energy differences due to steric strain or other contributors create a barrier to rotation that is high enough to allow for isolation of individual conformers. See, e.g., Bringmann, G. et al., Atroposelective Synthesis of Axially Chiral Biaryl Compounds. Angew. Chem., Int. Ed., 2005, 44:5384-5428. In some instances, the barrier of rotation is high enough that the different atropisomers may be separated and isolated, such as by chromatography on a chiral stationary phase. It is to be understood that the stereochemistry of the atropisomers is included in the compound names only when compounds are assayed as being pure (at least 95%) or are predominantly (at least 80%) one isomer. Where there is no atropisomer stereochemistry noted for a compound, then it is to be understood that either the stereochemistry is undetermined, or it was determined to be a near-equal mixture of atropisomers. In addition, where there is a discrepancy between the name of the compound and the structure found in Table 1, the structure depicted in Table 1 shall prevail.

Compounds of the present disclosure may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The present disclosure contemplates various stereoisomers and mixtures thereof and these are specifically included within the scope of this present disclosure. Stereoisomers include enantiomers and diastereomers, and mixtures of enantiomers or diastereomers. Individual stereoisomers of compounds of the present disclosure may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by precipitation or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Compounds of the present disclosure may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the present disclosure may be prepared synthetically from commercially available starting materials using selective organic transformations, or prepared in single isomeric form by purification of mixtures of the cis and trans isomers. Such methods are well-known to those of ordinary skill in the art, and may include separation of isomers by precipitation or chromatography.

It should be understood that the compounds of the disclosure may possess tautomeric forms, as well as geometric isomers, and that these also constitute an aspect of the disclosure.

The present disclosure includes all pharmaceutically acceptable isotopically-labeled compounds of Formula (I) wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labeled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. 2H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I) may generally be prepared by conventional techniques known to those skilled in the art or by processes analogous to those described in the accompanying Examples using an appropriate isotopically-labeled reagents in place of the non-labeled reagent previously employed.

Thus, the formula drawings within this specification can represent only one of the possible tautomeric, geometric, or stereoisomeric forms. It is to be understood that the present disclosure encompasses any tautomeric, geometric, or stereoisomeric form, and mixtures thereof, and is not to be limited merely to any one tautomeric, geometric, or stereoisomeric form utilized within the formula drawings.

Exemplary compounds of Formula (I) include, but are not limited to, the compounds shown in Table 1 below. It is to be understood that when there is a discrepancy between the name of the compound found herein and the structure found in Table I, the structure in Table 1 shall prevail. In addition, it is to be understood that an asterisk (*), at a particular stereocenter in a structure, indicates an arbitrary assignment of stereochemical configuration at that stereocenter.

TABLE 1

| Ex | Structure |
|---|---|
| 1 | 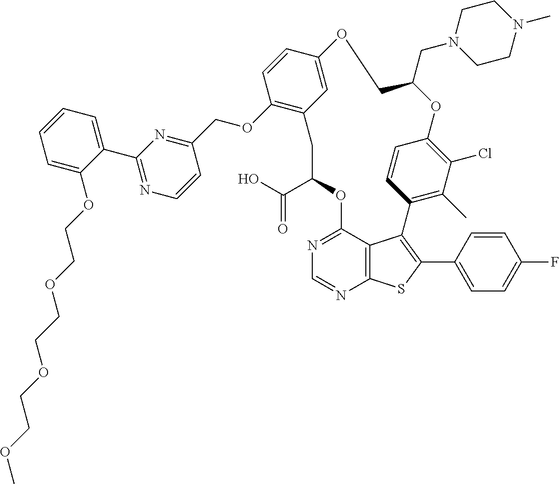 |
| 2 | 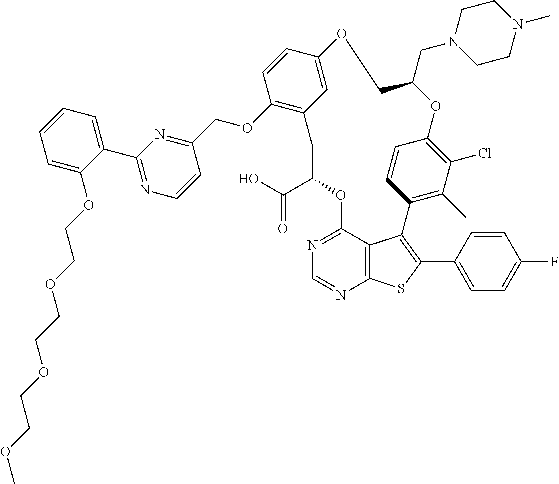 |
| 3 | 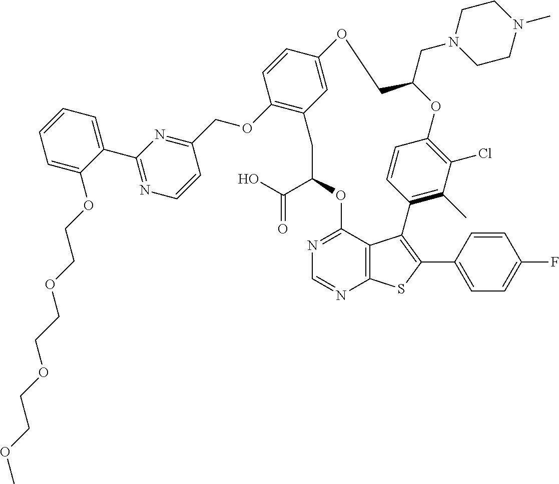 [SMILES: COCCOCCOCCOc1ccccc1-c1nccc(COc2ccc3cc2C[C@H](C(=O)O)Oc2ncnc4sc(-c5ccc(F)cc5)c(c24)-c2ccc(c(Cl)c2C)O[C@H](CN2CCN(C)CC2)CO3)n1] |
| 4 | 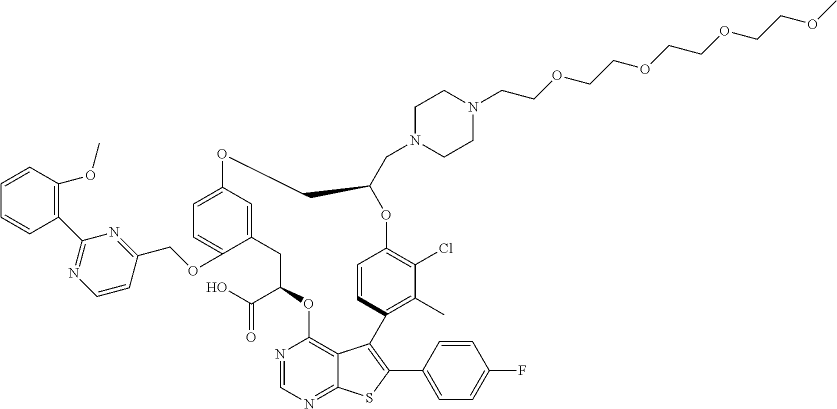 |
| 5 | 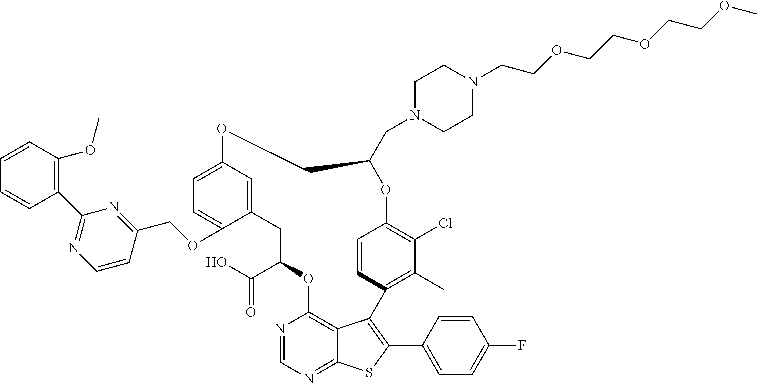 |
| 6 | 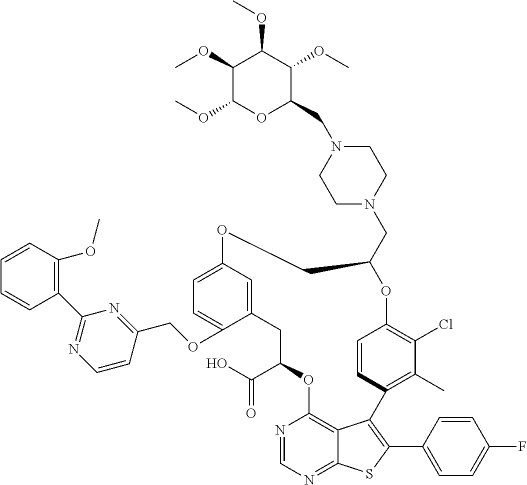 |
| 7 | 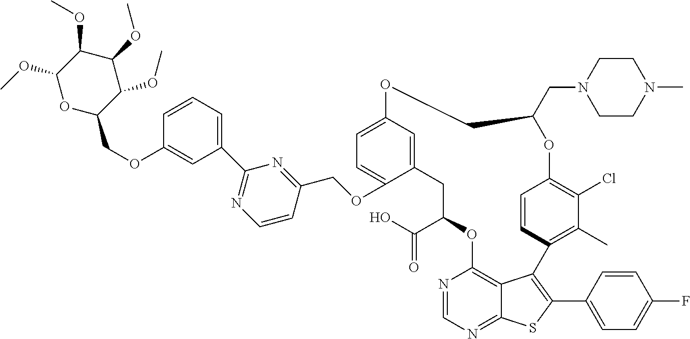 |
| 8 | 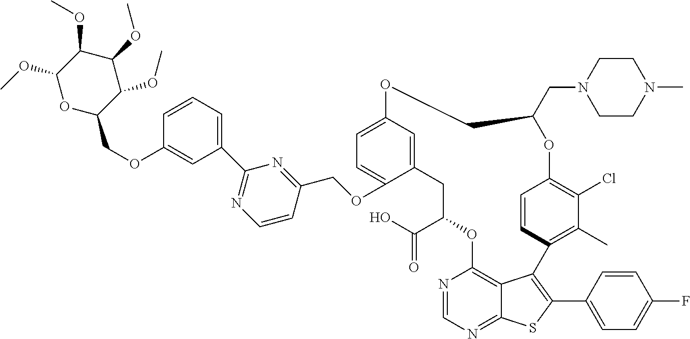 |
| 9 | 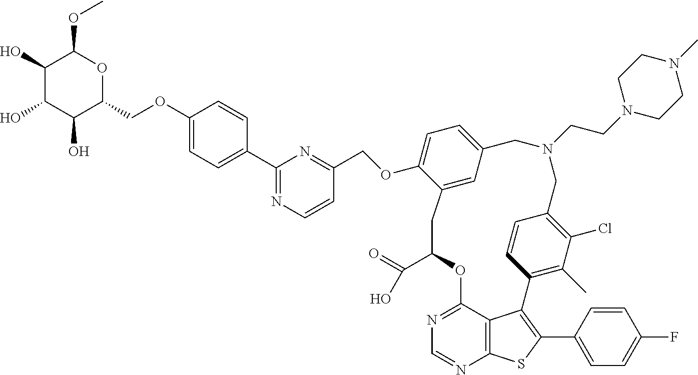 |
| 10 | 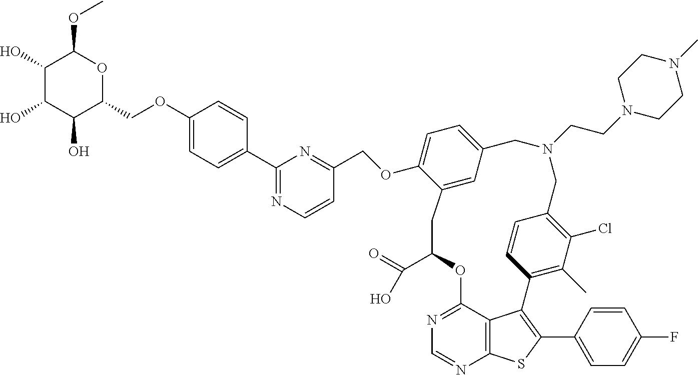 |
| 11 | 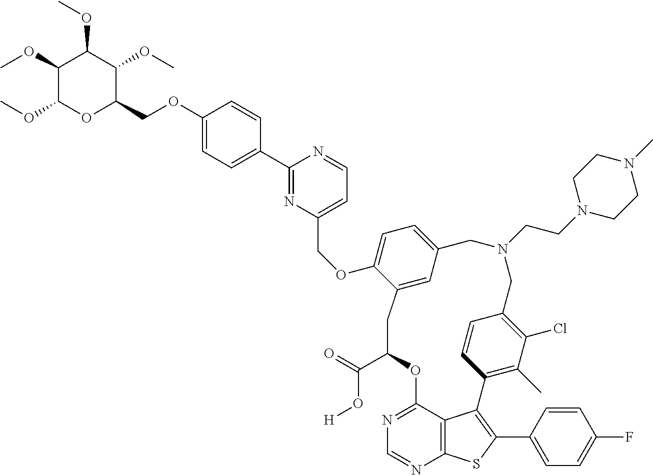 |
| 12 | 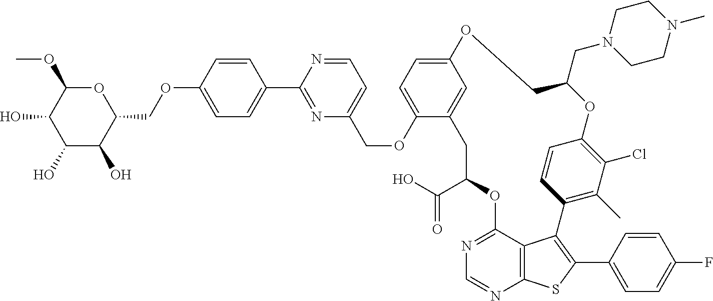 |
| 13 | 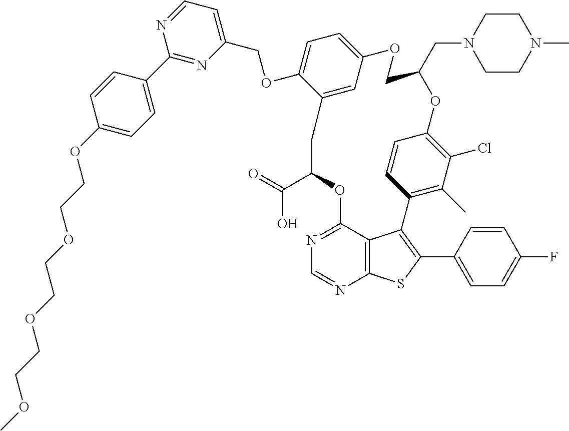 |
| 14 | 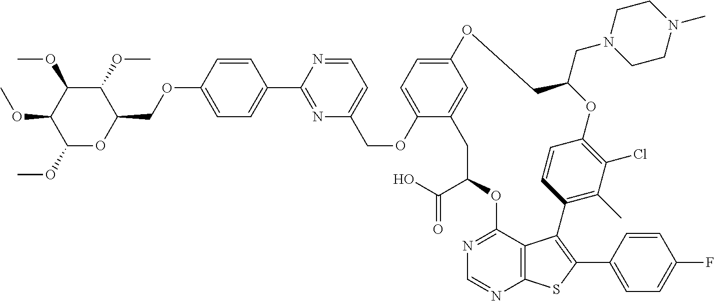 |
| 15 | 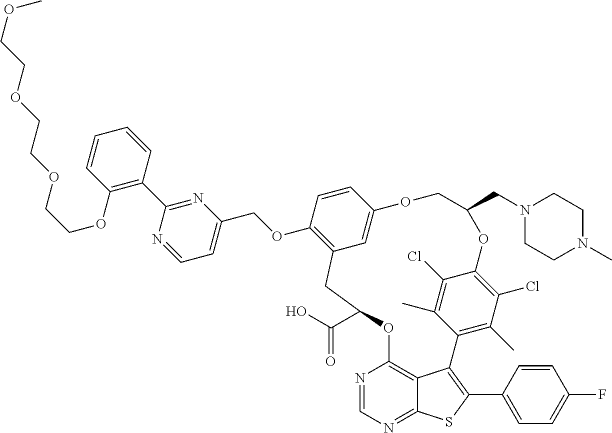 [SMILES: COCCOCCOCCOc1ccccc1-c1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 16 | 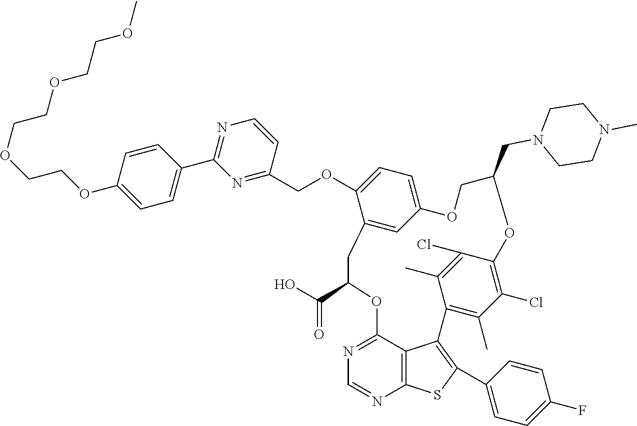 [SMILES: COCCOCCOCCOc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1] |
| 17 | 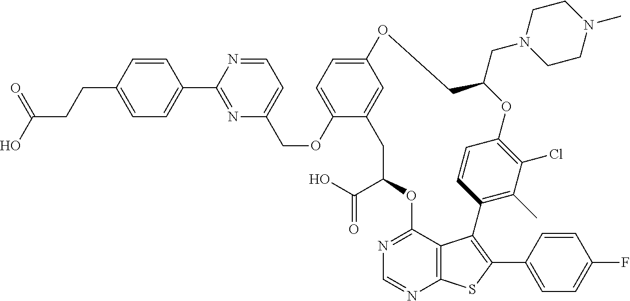 |
| 18 | 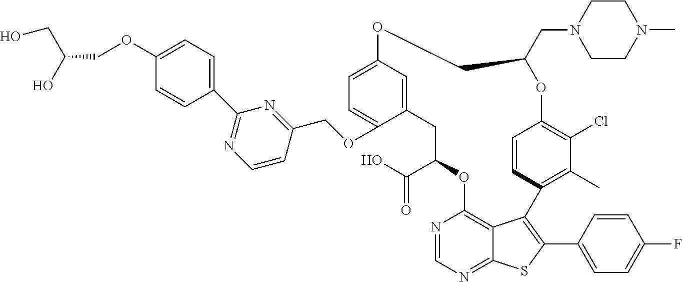 |
| 19 | 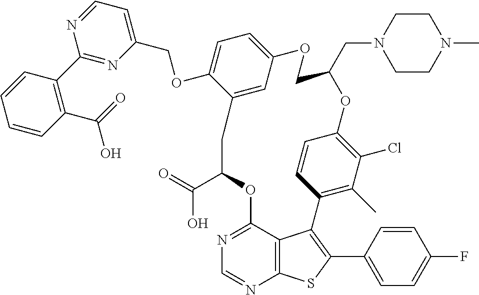 |
| 20 | 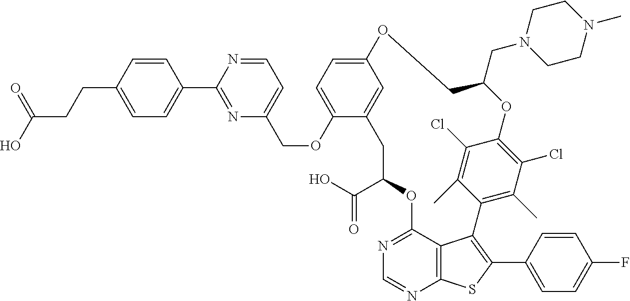 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(CCC(=O)O)cc3)n1)OC[C@@H](CN1CCN(C)CC1)O2] |
| 21 | 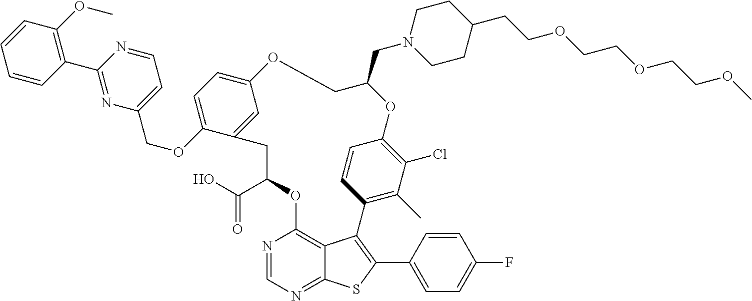 |
| 22 | 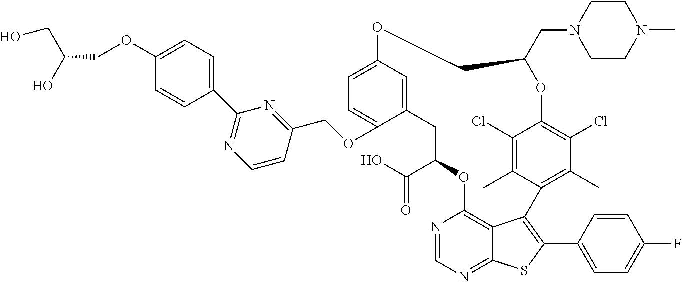 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OC[C@H](O)CO)cc3)n1)OC[C@@H](CN1CCN(C)CC1)O2] |
| 23 | 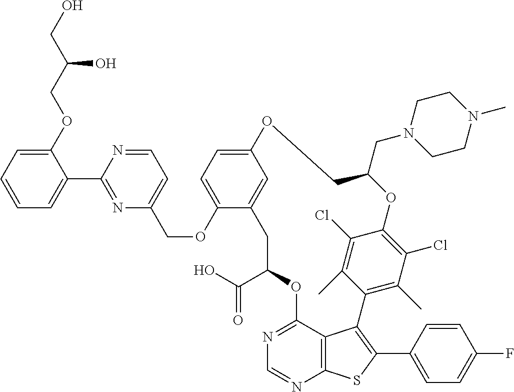 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccccc3OC[C@H](O)CO)n1)OC[C@@H](CN1CCN(C)CC1)O2] |
| 24 | 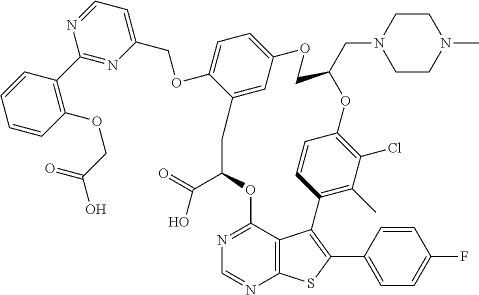 |
| 25 | 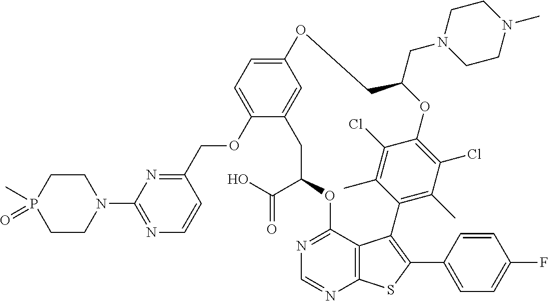 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCP(C)(=O)CC3)n1)OC[C@@H](CN1CCN(C)CC1)O2] |
| 26 | 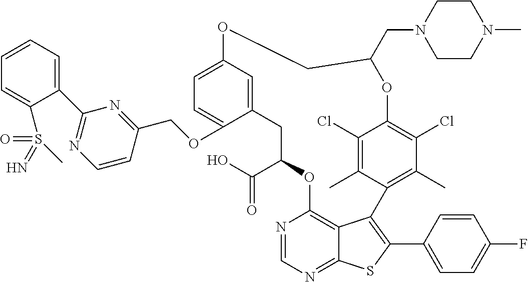 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccccc3S(C)(=N)=O)n1)OCC(CN1CCN(C)CC1)O2] |
| 27 | 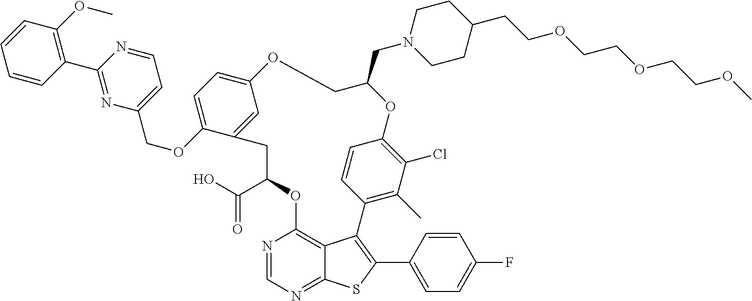 [SMILES: COCCOCCOCCC1CCN(C[C@@H]2COc3ccc(OCc4ccnc(-c5ccccc5OC)n4)c(c3)C[C@H](C(=O)O)Oc3ncnc4sc(-c5ccc(F)cc5)c(c34)-c3ccc(c(Cl)c3C)O2)CC1] |
| 28 | 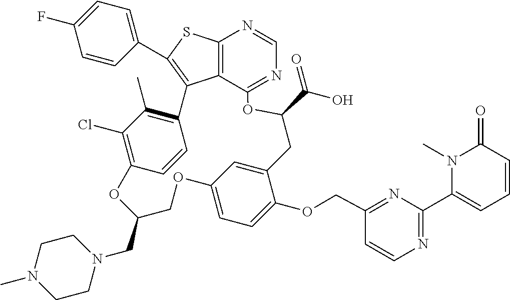 [SMILES: Cc1c2ccc(c1Cl)O[C@H](CN1CCN(C)CC1)COc1ccc(OCc3ccnc(-c4cccc(=O)n4C)n3)c(c1)C[C@H](C(=O)O)Oc1ncnc3sc(-c4ccc(F)cc4)c-2c13] |
| 29 | 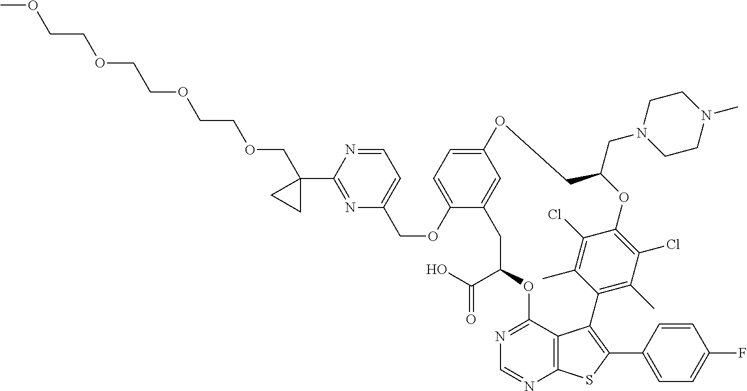 [SMILES: COCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 30 | 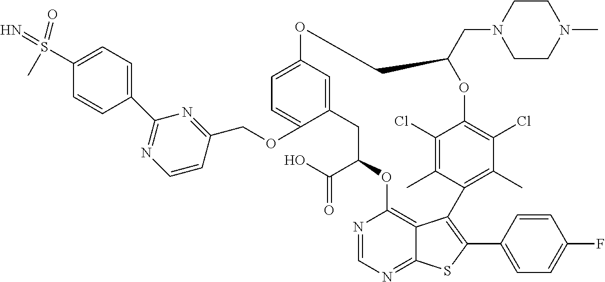 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(S(C)(=N)=O)cc3)n1)OC[C@@H](CN1CCN(C)CC1)O2] |
| 31 | 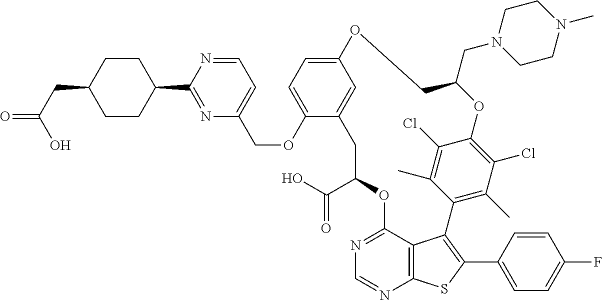 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc([C@H]3CC[C@@H](CC(=O)O)CC3)n1)OC[C@@H](CN1CCN(C)CC1)O2] |
| 32 | 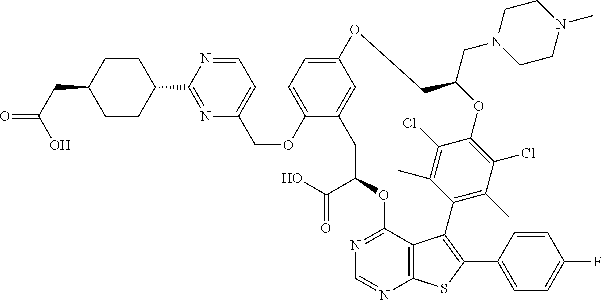 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc([C@H]3CC[C@H](CC(=O)O)CC3)n1)OC[C@@H](CN1CCN(C)CC1)O2] |
| 33 | 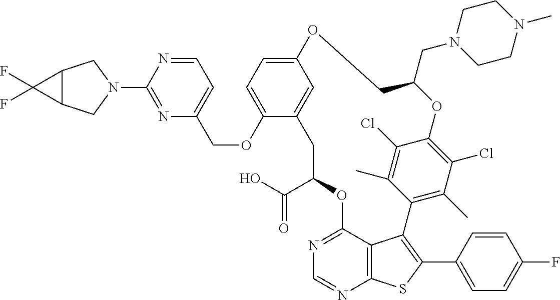 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CC4C(C3)C4(F)F)n1)OC[C@@H](CN1CCN(C)CC1)O2] |
| 34 | 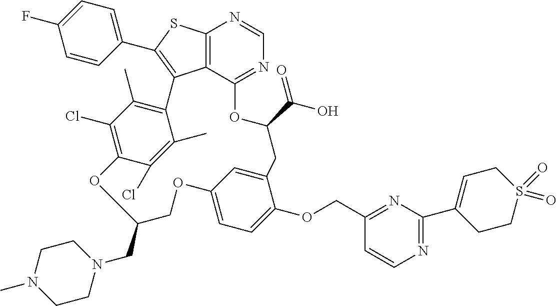 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C3=CCS(=O)(=O)CC3)n1)OC[C@@H](CN1CCN(C)CC1)O2] |
| 35 | 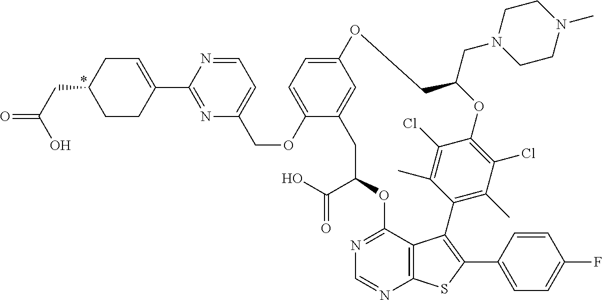 |
| 36 | 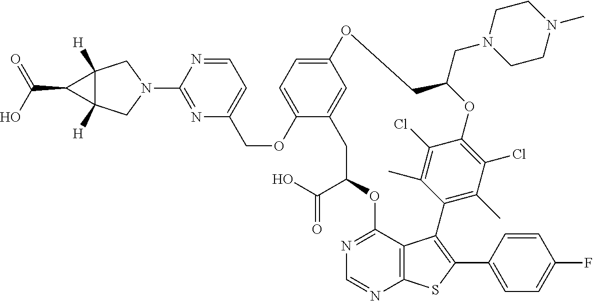 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3C[C@@H]4[C@H](C3)[C@H]4C(=O)O)n1)OC[C@@H](CN1CCN(C)CC1)O2] |
| 37 | 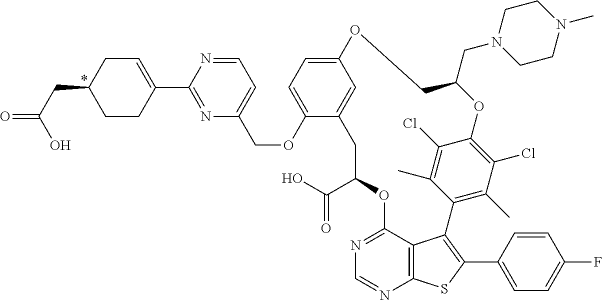 |
| 38 | 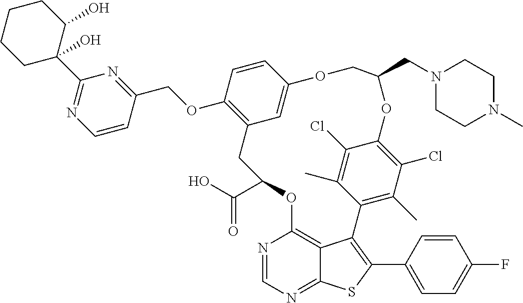 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc([C@@]3(O)CCCC[C@@H]3O)n1)OC[C@@H](CN1CCN(C)CC1)O2] |
| 39 | 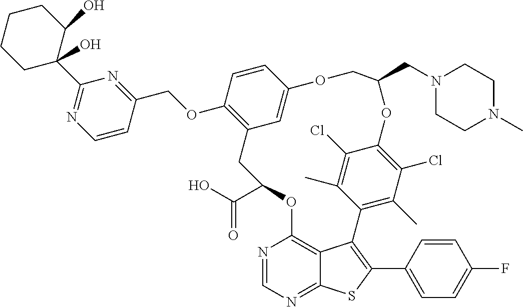 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc([C@]3(O)CCCC[C@H]3O)n1)OC[C@@H](CN1CCN(C)CC1)O2] |
| 40 | 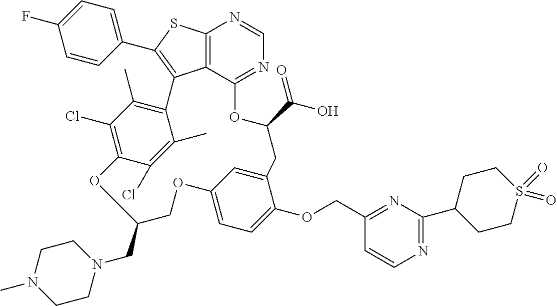 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C3CCS(=O)(=O)CC3)n1)OC[C@@H](CN1CCN(C)CC1)O2] |
| 41 | 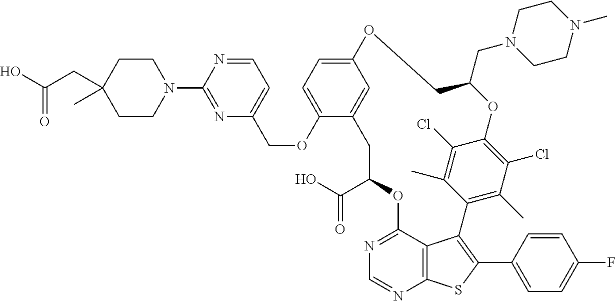 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCC(C)(CC(=O)O)CC3)n1)OC[C@@H](CN1CCN(C)CC1)O2] |
| 42 | 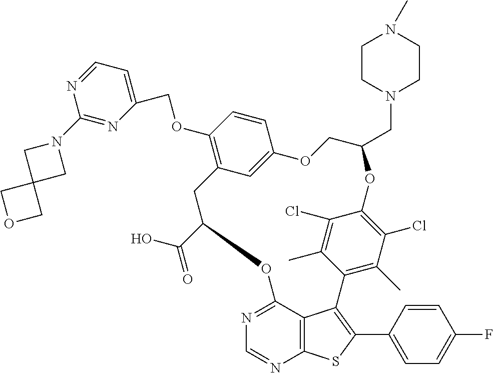 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CC4(COC4)C3)n1)OC[C@@H](CN1CCN(C)CC1)O2] |
| 43 | 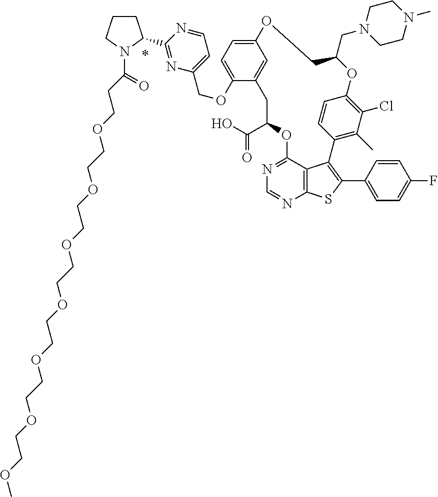 |
| 44 | 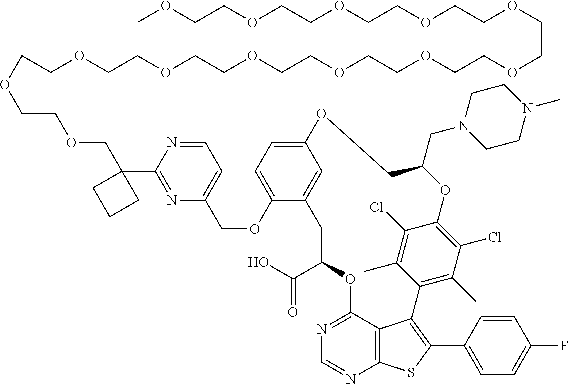 [SMILES: COCCOCCOCCOCCOCCOCCOCCOCCOCCOCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1] |
| 45 | 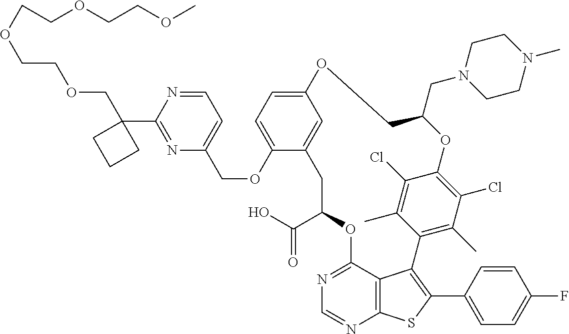 [SMILES: COCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1] |
| 46 | 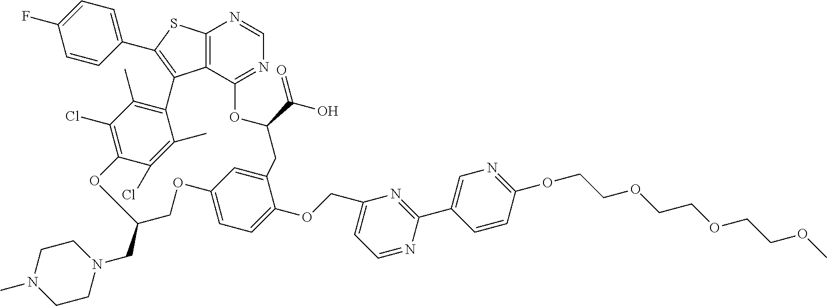 |
| 47 | 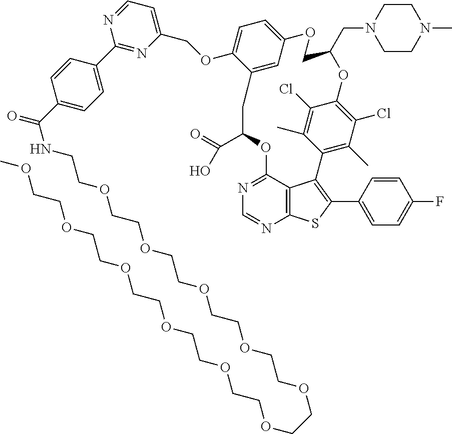 |
| 48 | 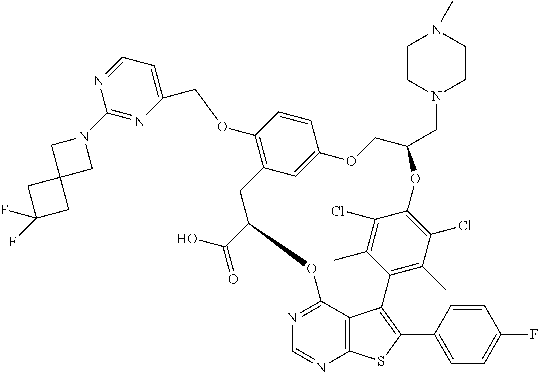 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CC4(C3)CC(F)(F)C4)n1)OC[C@@H](CN1CCN(C)CC1)O2] |
| 49 | 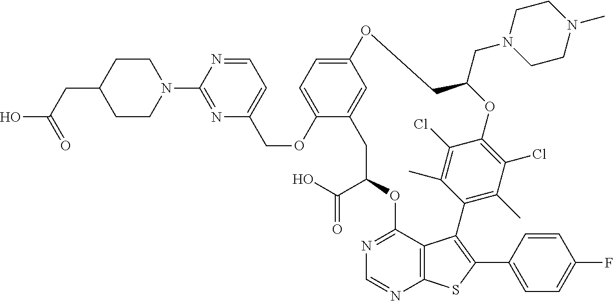 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCC(CC(=O)O)CC3)n1)OC[C@@H](CN1CCN(C)CC1)O2] |
| 50 | 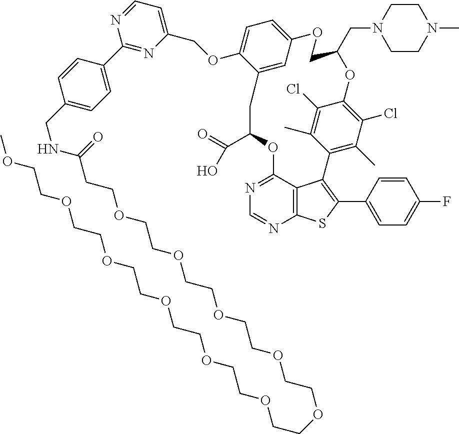 |
| 51 | 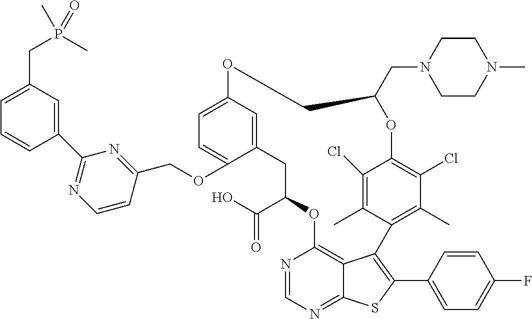 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3cccc(CP(C)(C)=O)c3)n1)OC[C@@H](CN1CCN(C)CC1)O2] |
| 52 | 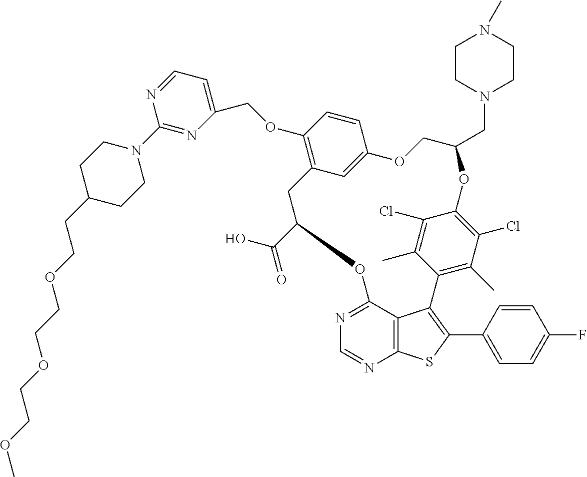 [SMILES: COCCOCCOCCC1CCN(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 53 | 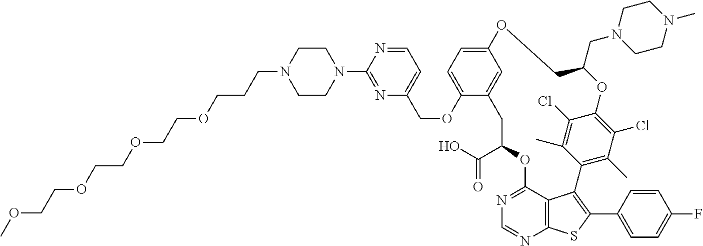 [SMILES: COCCOCCOCCOCCCN1CCN(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 54 | 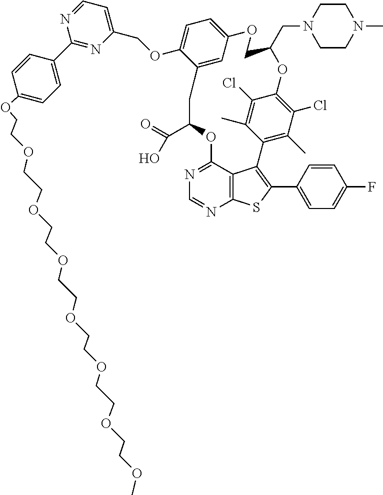 [SMILES: COCCOCCOCCOCCOCCOCCOCCOc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1] |
| 55 | 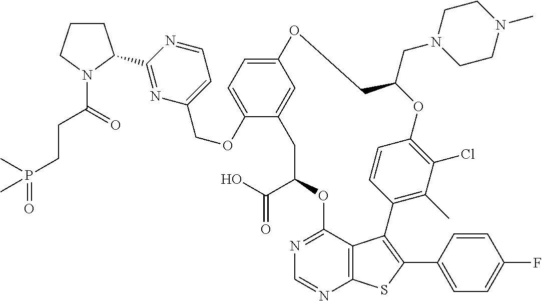 [SMILES: Cc1c2ccc(c1Cl)O[C@H](CN1CCN(C)CC1)COc1ccc(OCc3ccnc([C@H]4CCCN4C(=O)CCP(C)(C)=O)n3)c(c1)C[C@H](C(=O)O)Oc1ncnc3sc(-c4ccc(F)cc4)c-2c13] |
| 56 | 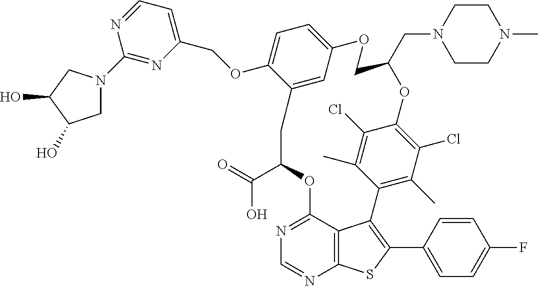 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3C[C@H](O)[C@@H](O)C3)n1)OC[C@@H](CN1CCN(C)CC1)O2] |
| 57 | 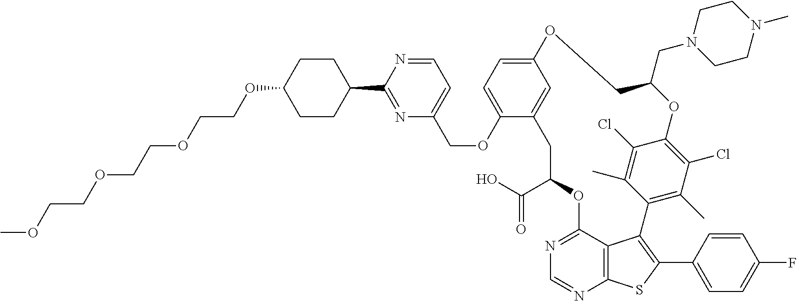 [SMILES: COCCOCCOCCO[C@H]1CC[C@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 58 | 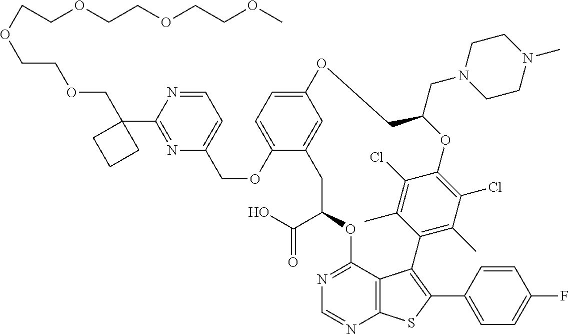 [SMILES: COCCOCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1] |
| 59 | 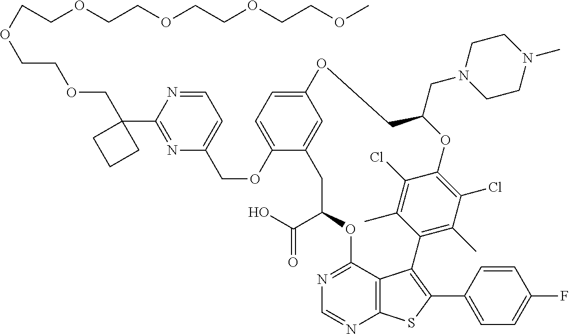 [SMILES: COCCOCCOCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1] |
| 60 | 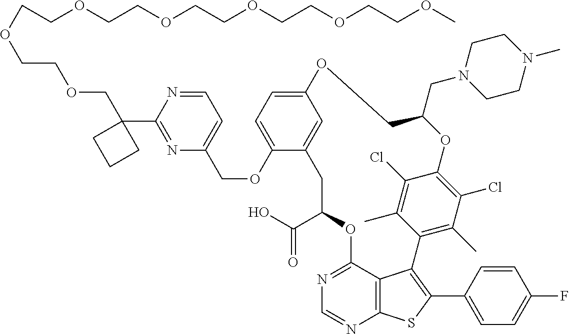 [SMILES: COCCOCCOCCOCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1] |
| 61 | 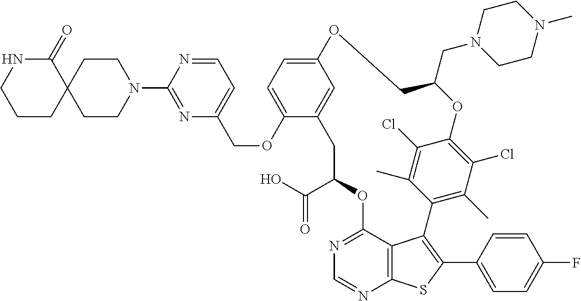 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCC4(CCCNC4=O)CC3)n1)OC[C@@H](CN1CCN(C)CC1)O2] |
| 62 | 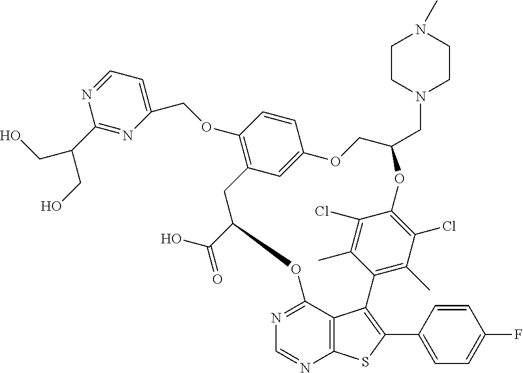 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C(CO)CO)n1)OC[C@@H](CN1CCN(C)CC1)O2] |
| 63 | 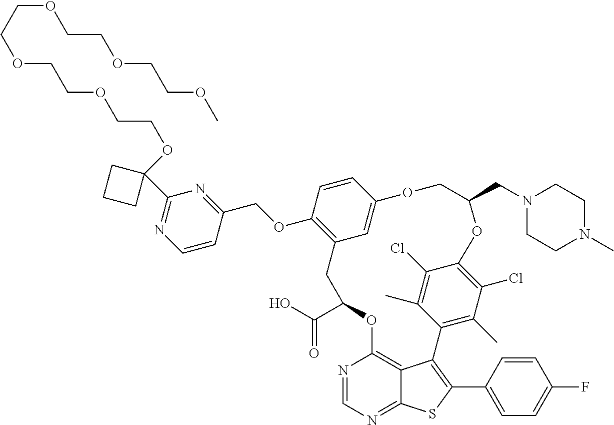 [SMILES: COCCOCCOCCOCCOCCOC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1] |
| 64 | 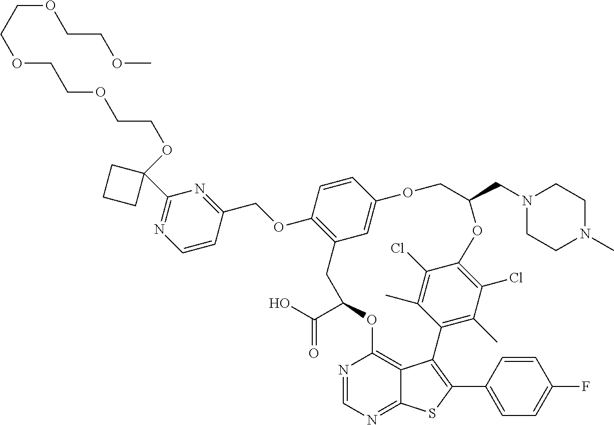 [SMILES: COCCOCCOCCOCCOC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1] |
| 65 | 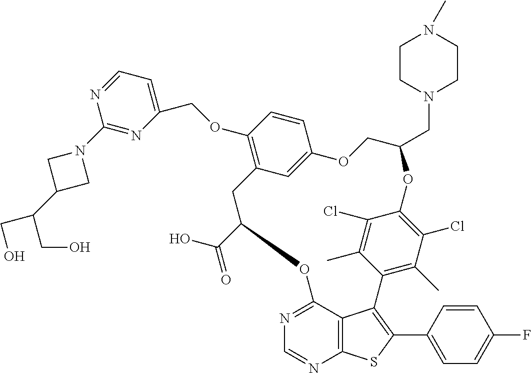 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CC(C(CO)CO)C3)n1)OC[C@@H](CN1CCN(C)CC1)O2] |
| 66 | 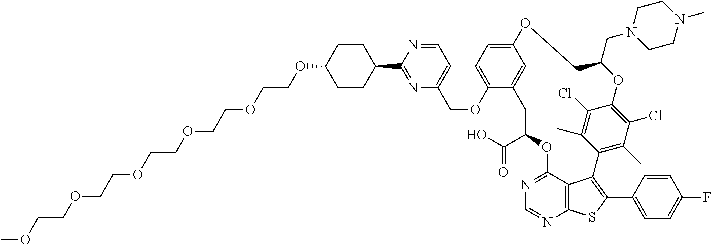 [SMILES: COCCOCCOCCOCCOCCO[C@H]1CC[C@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 67 | 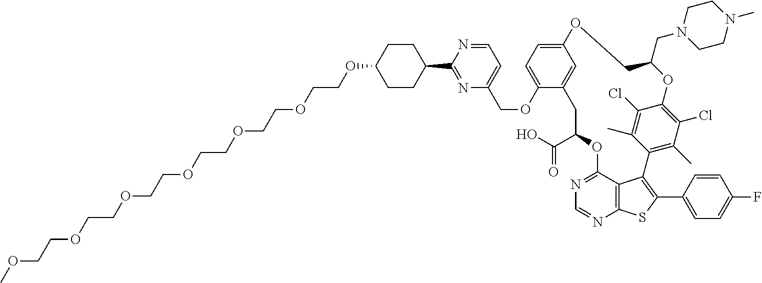 [SMILES: COCCOCCOCCOCCOCCOCCO[C@H]1CC[C@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 68 | 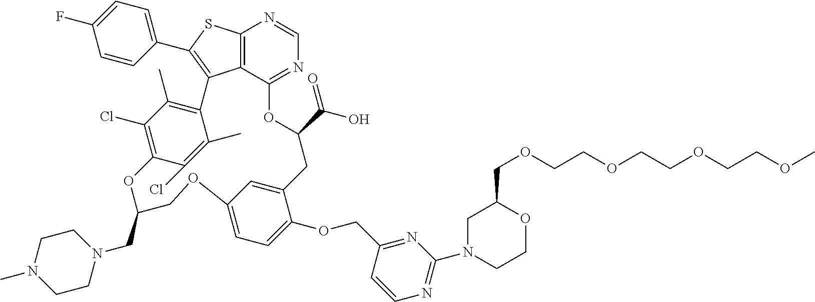 |
| 69 | 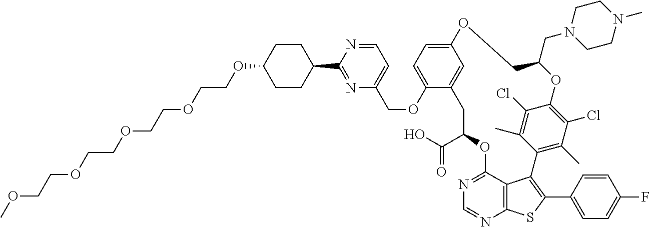 [SMILES: COCCOCCOCCOCCO[C@H]1CC[C@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 70 | 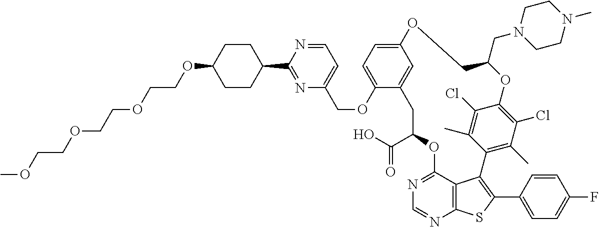 [SMILES: COCCOCCOCCO[C@H]1CC[C@@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 71 | 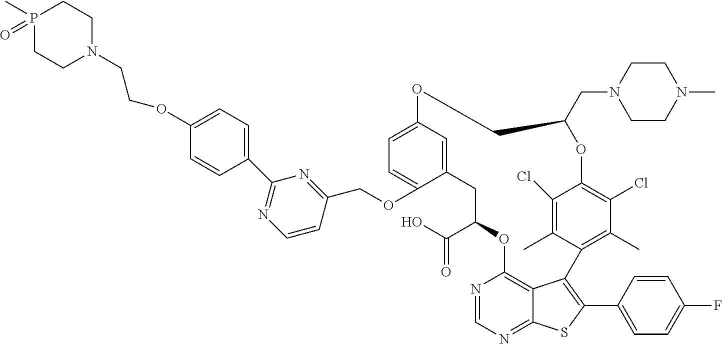 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OCCN4CCP(C)(=O)CC4)cc3)n1)OC[C@@H](CN1CCN(C)CC1)O2] |
| 72 | 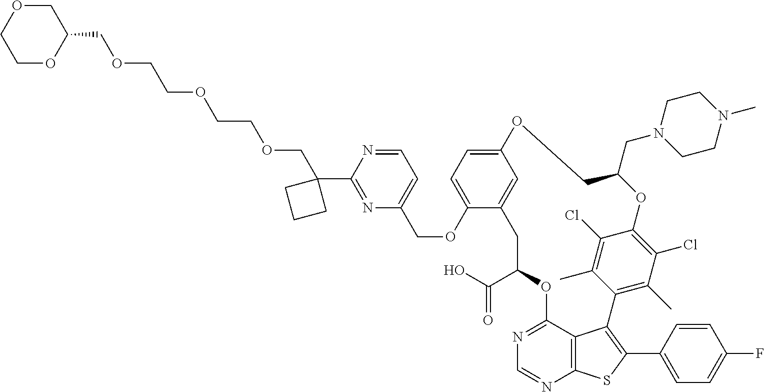 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C3(COCCOCCOC[C@H]4COCCO4)CCC3)n1)OC[C@@H](CN1CCN(C)CC1)O2] |
| 73 | 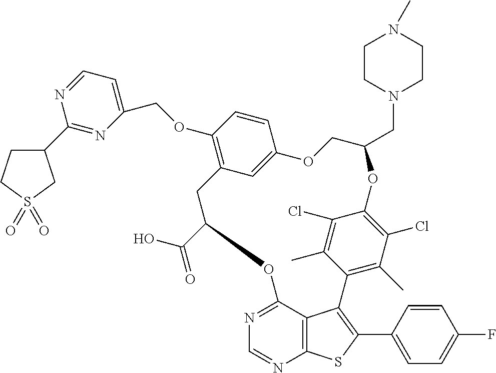 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C3CCS(=O)(=O)C3)n1)OC[C@@H](CN1CCN(C)CC1)O2] |
| 74 | 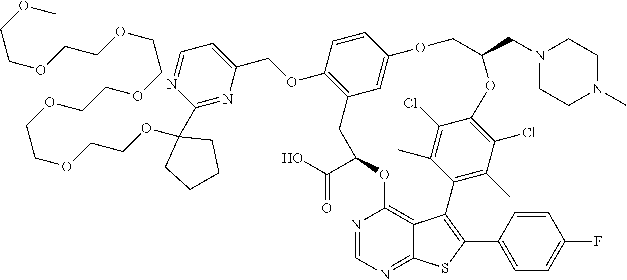 [SMILES: COCCOCCOCCOCCOCCOCCOC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCCC1] |
| 75 | 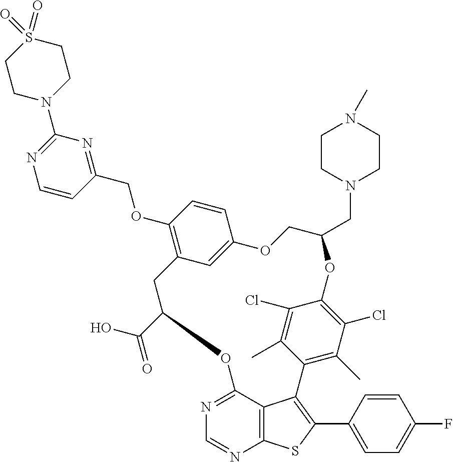 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCS(=O)(=O)CC3)n1)OC[C@@H](CN1CCN(C)CC1)O2] |
| 76 | 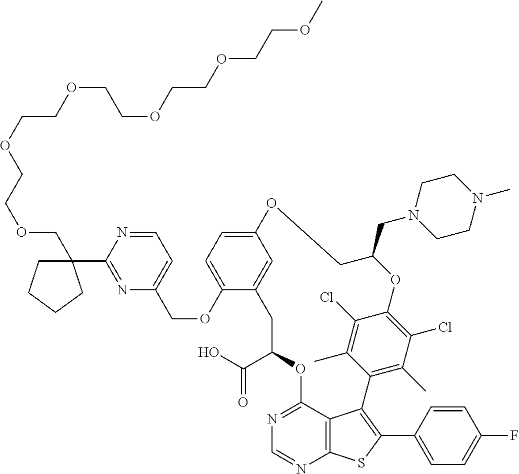 [SMILES: COCCOCCOCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCCC1] |
| 77 | 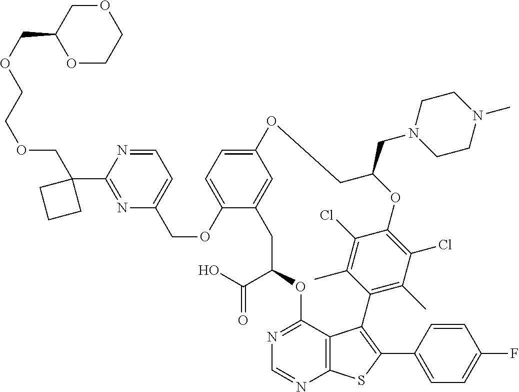 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C3(COCCOC[C@H]4COCCO4)CCC3)n1)OC[C@@H](CN1CCN(C)CC1)O2] |
| 78 | 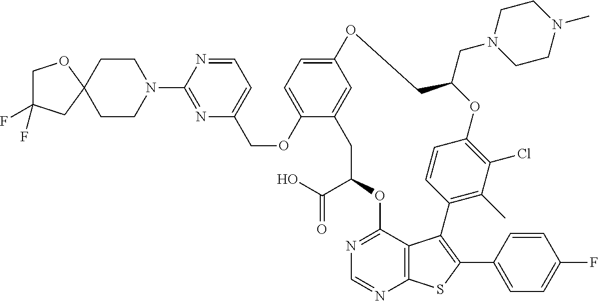 [SMILES: Cc1c2ccc(c1Cl)O[C@H](CN1CCN(C)CC1)COc1ccc(OCc3ccnc(N4CCC5(CC4)CC(F)(F)CO5)n3)c(c1)C[C@H](C(=O)O)Oc1ncnc3sc(-c4ccc(F)cc4)c-2c13] |
| 79 | 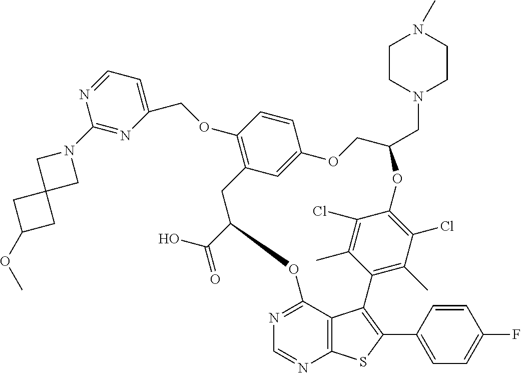 [SMILES: COC1CC2(C1)CN(c1nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n1)C2] |
| 80 | 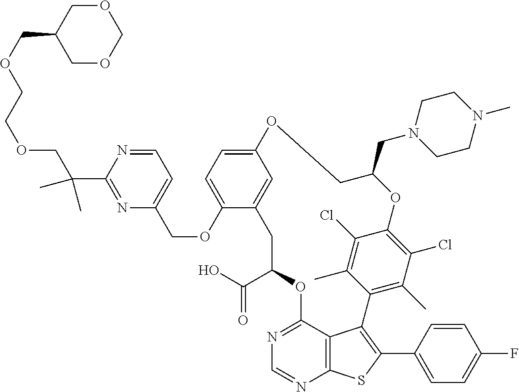 |
| 81 | 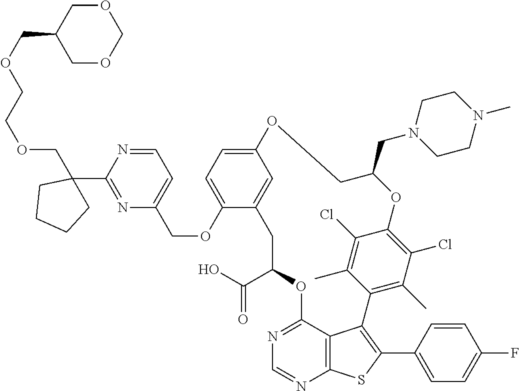 |
| 82 | 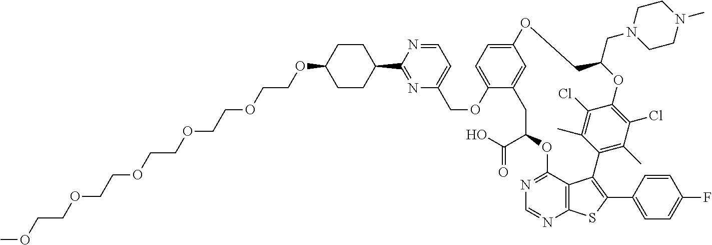 [SMILES: COCCOCCOCCOCCOCCO[C@H]1CC[C@@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 83 | 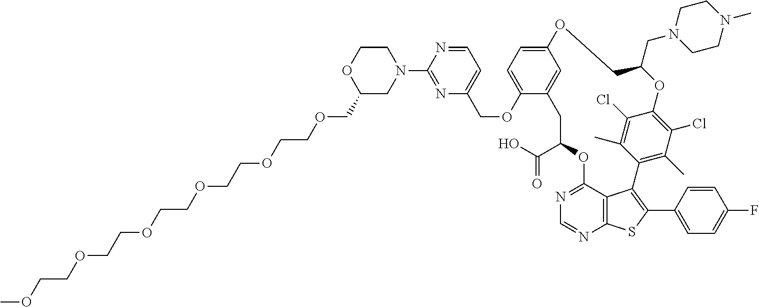 [SMILES: COCCOCCOCCOCCOCCOC[C@H]1CN(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCO1] |
| 84 | 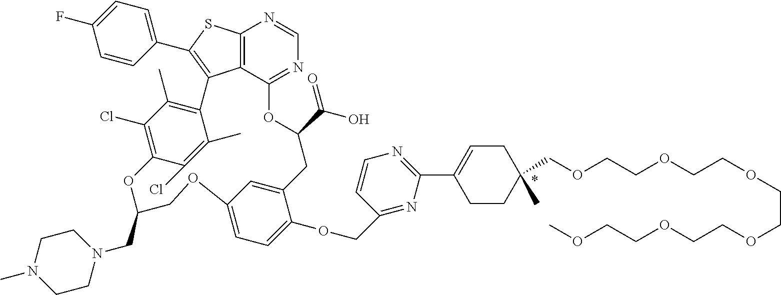 |
| 85 | 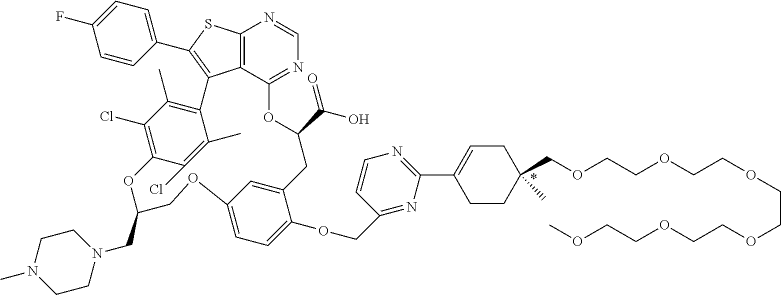 |
| 86 | 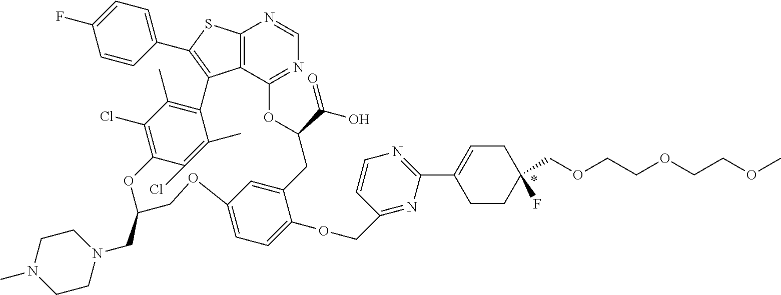 |
| 87 | 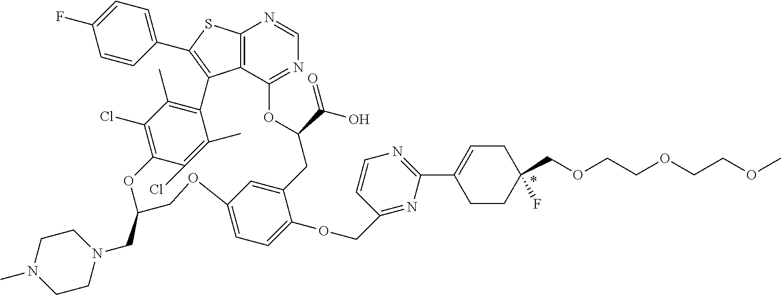 |
| 88 | 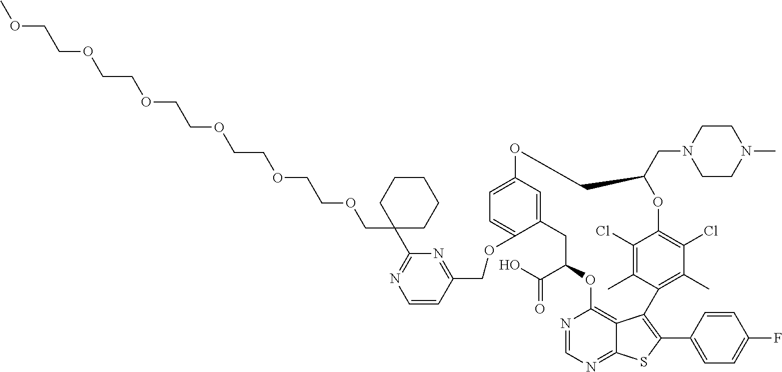 [SMILES: COCCOCCOCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCCCC1] |
| 89 | 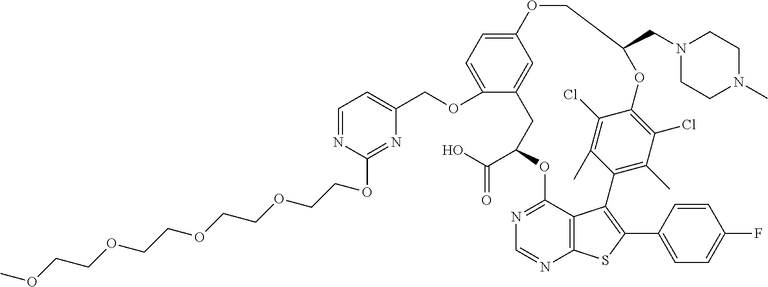 |
| 90 | 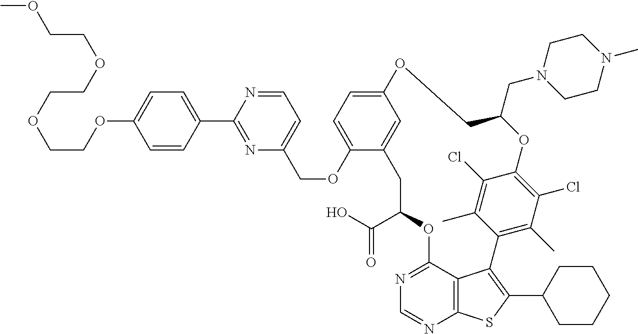 |
| 91 | 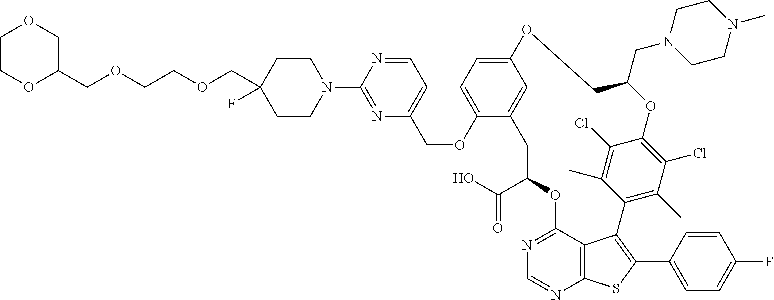 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCC(F)(COCCOCC4COCCO4)CC3)n1)OC[C@@H](CN1CCN(C)CC1)O2] |
| 92 | 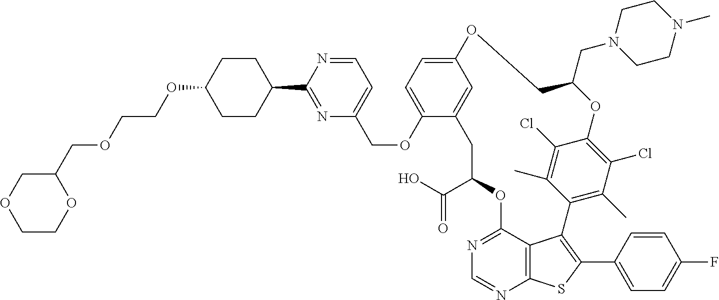 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc([C@H]3CC[C@H](OCCOCC4COCCO4)CC3)n1)OC[C@@H](CN1CCN(C)CC1)O2] |
| 93 | 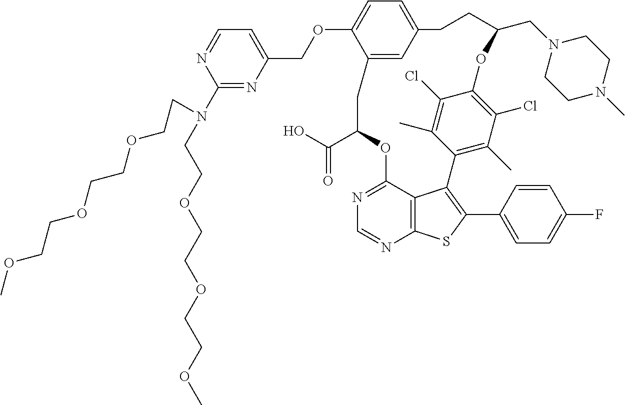 |
| 94 | 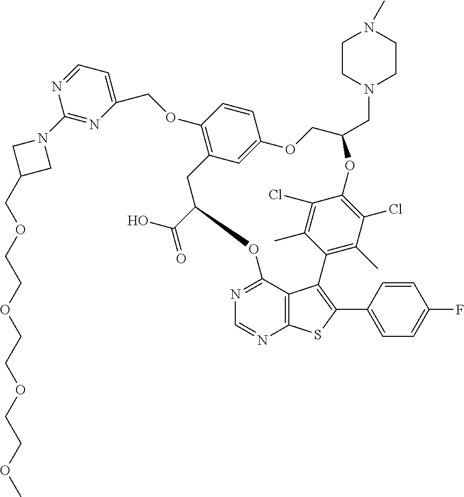 [SMILES: COCCOCCOCCOCC1CN(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)C1] |
| 95 | 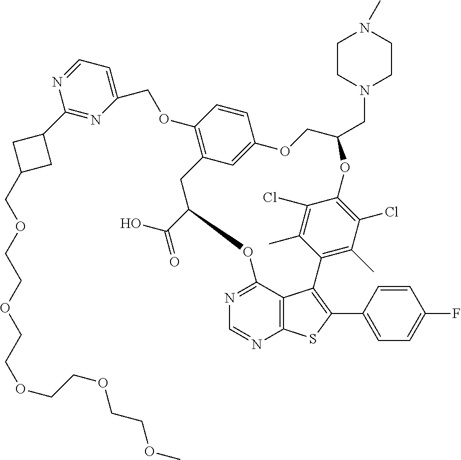 [SMILES: COCCOCCOCCOCCOCC1CC(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)C1] |
| 96 | 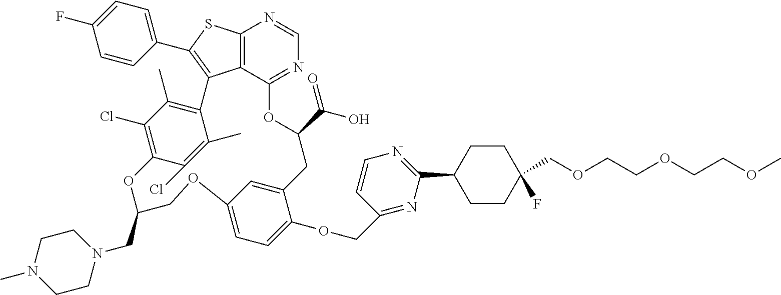 |
| 97 | 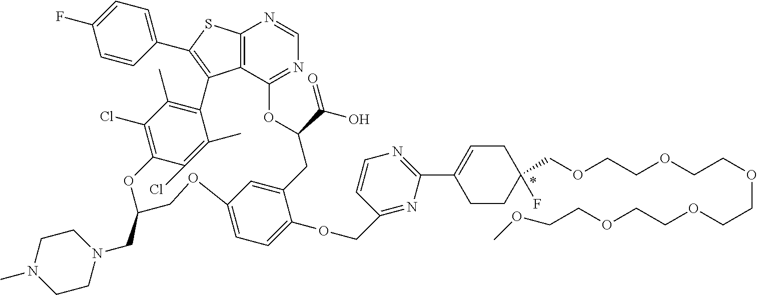 |
| 98 | 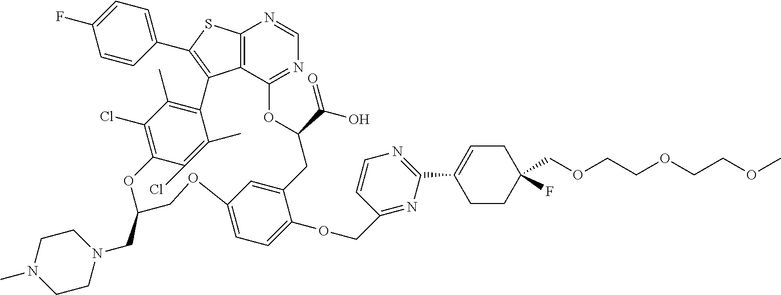 |
| 99 | 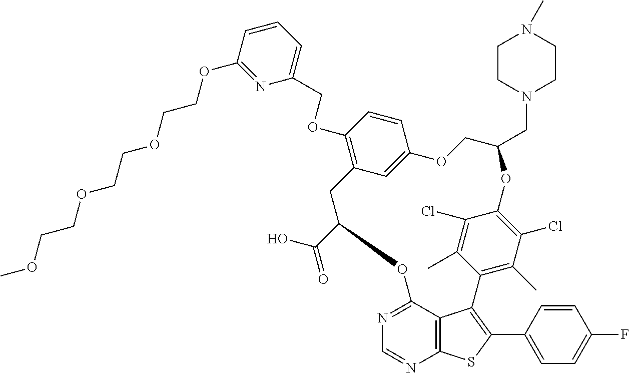 [SMILES: COCCOCCOCCOc1cccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 100 | 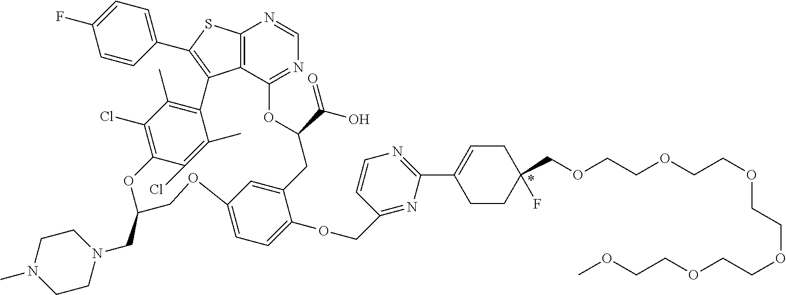 |
| 101 | 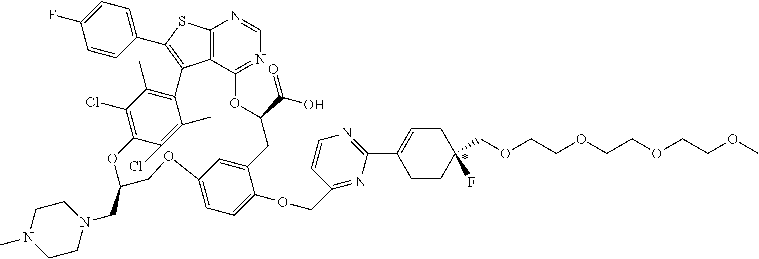 |
| 102 | 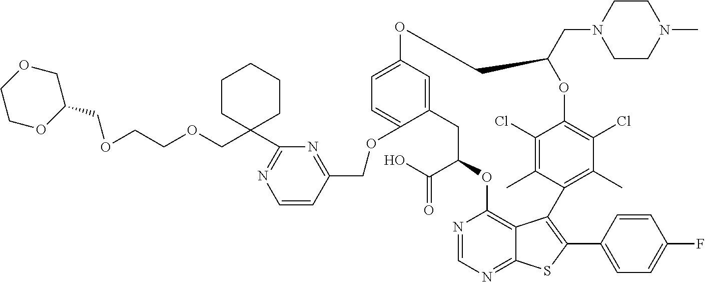 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C3(COCCOC[C@H]4COCCO4)CCCCC3)n1)OC[C@@H](CN1CCN(C)CC1)O2] |
| 103 | 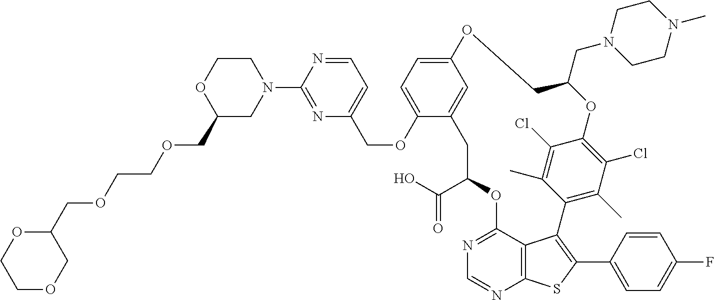 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCO[C@H](COCCOCC4COCCO4)C3)n1)OC[C@@H](CN1CCN(C)CC1)O2] |
| 104 | 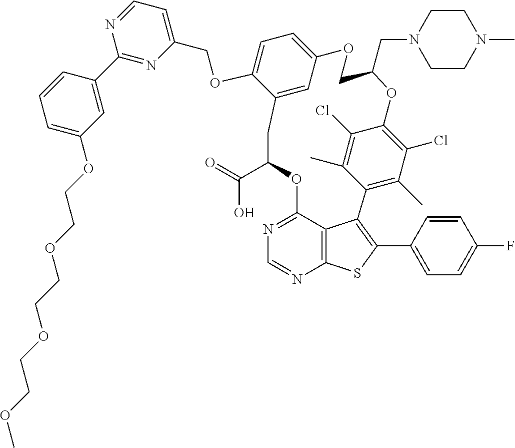 [SMILES: COCCOCCOCCOc1cccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)c1] |
| 105 | 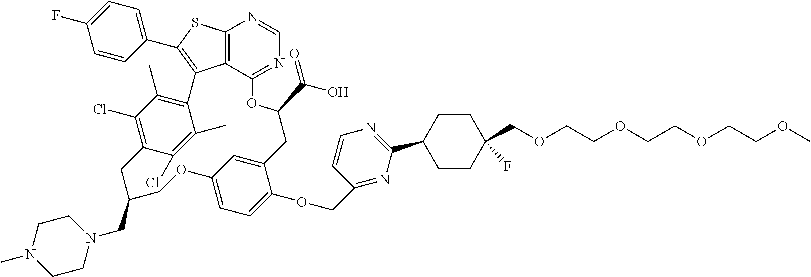 |
| 106 | 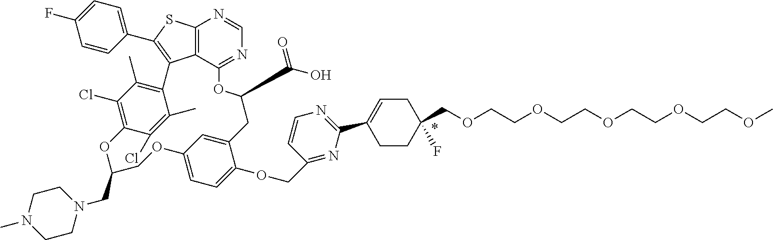 |
| 107 | 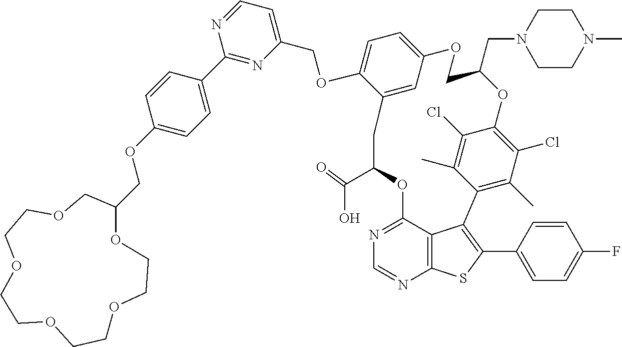 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OCC4COCCOCCOCCOCCO4)cc3)n1)OC[C@@H](CN1CCN(C)CC1)O2] |
| 108 | 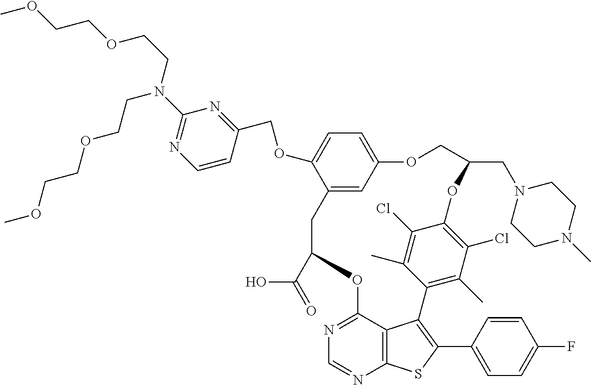 |
| 109 | 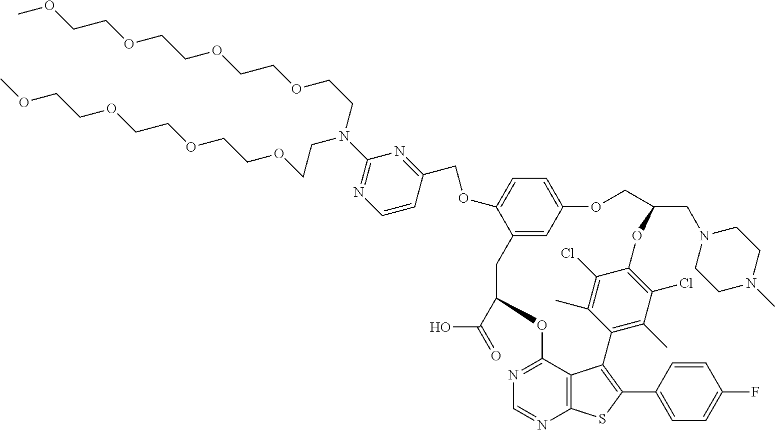 |
| 110 | 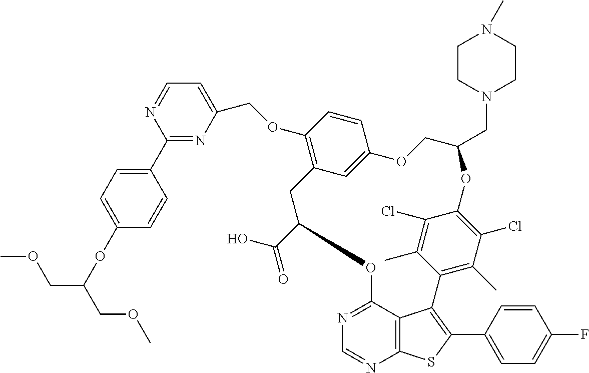 [SMILES: COCC(COC)Oc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1] |
| 111 | 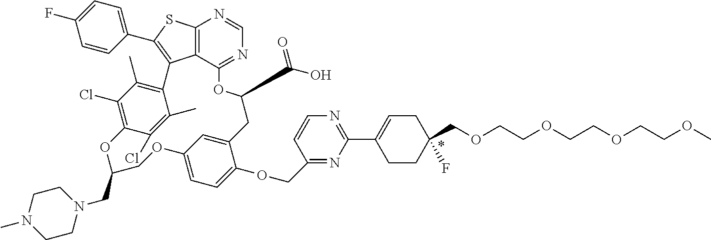 |
| 112 | 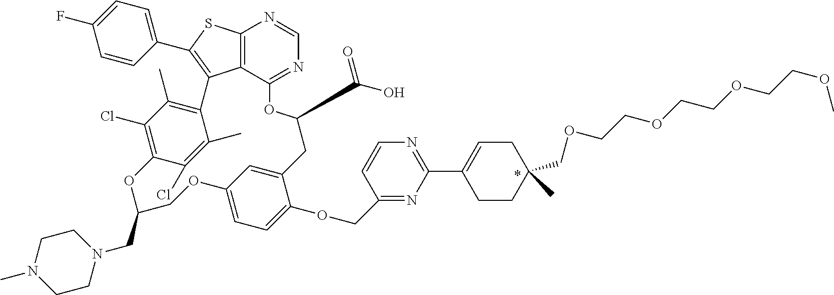 |
| 113 | 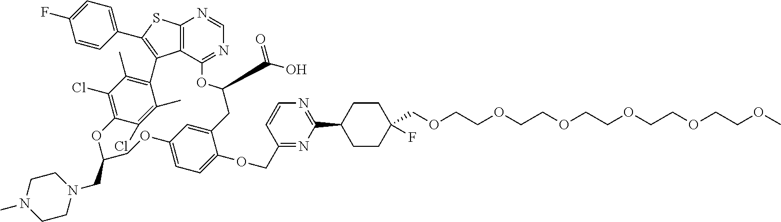 |
| 114 | 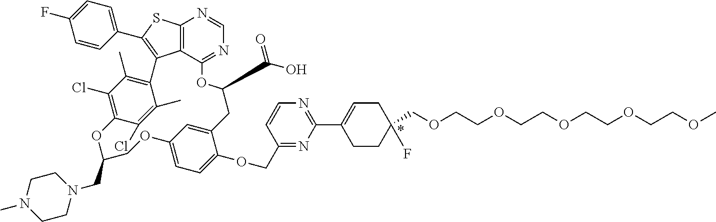 |
| 115 | 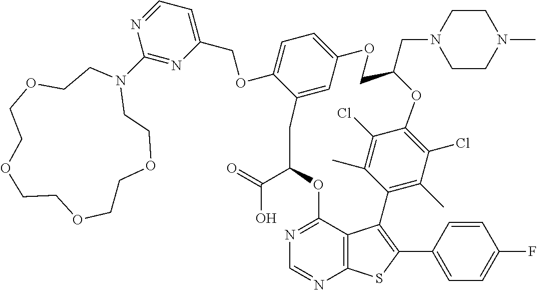 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCOCCOCCOCCOCC3)n1)OC[C@@H](CN1CCN(C)CC1)O2] |
| 116 | 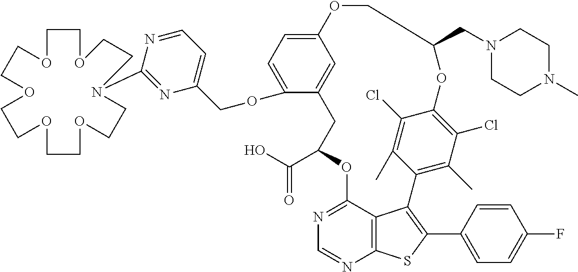 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCOCCOCCOCCOCCOCC3)n1)OC[C@@H](CN1CCN(C)CC1)O2] |
| 117 | 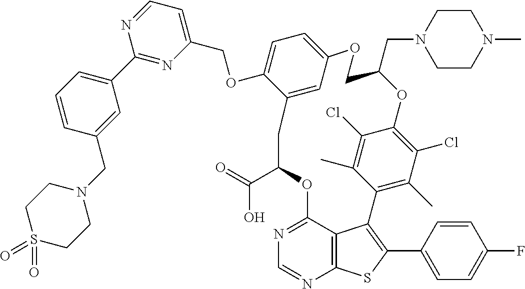 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3cccc(CN4CCS(=O)(=O)CC4)c3)n1)OC[C@@H](CN1CCN(C)CC1)O2] |
| 118 | 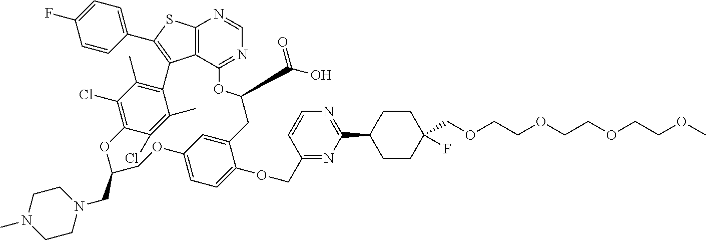 |
| 119 | 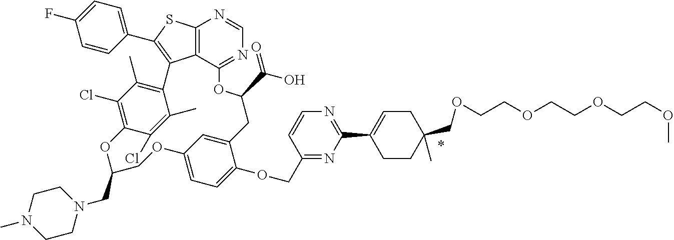 |
| 120 | 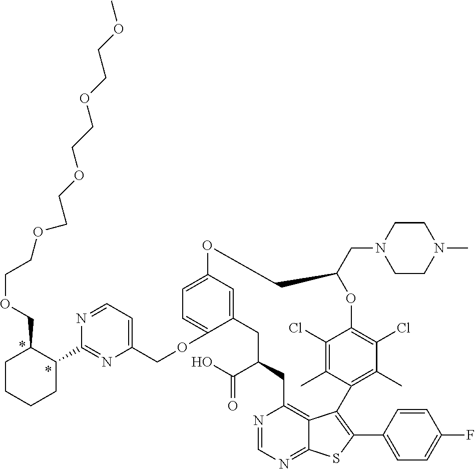 |
| 121 | 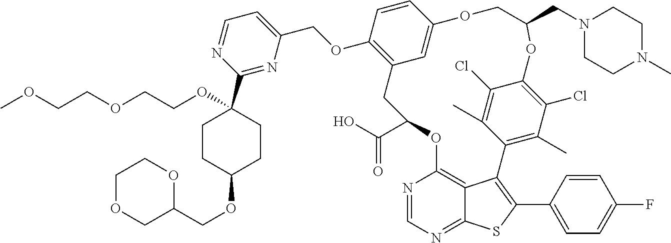 |
| 122 | 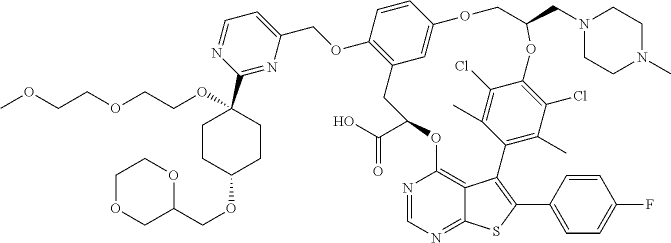 |
| 123 | 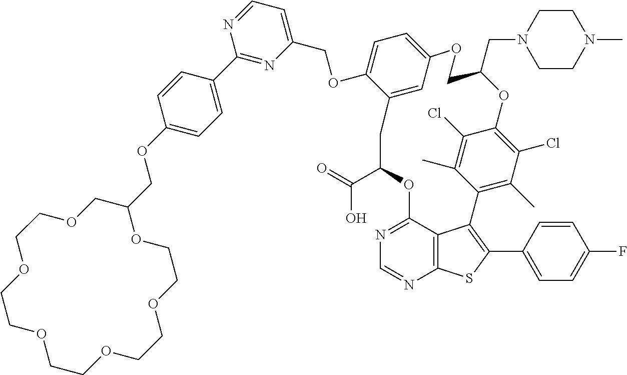 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OCC4COCCOCCOCCOCCOCCO4)cc3)n1)OC[C@@H](CN1CCN(C)CC1)O2] |
| 124 | 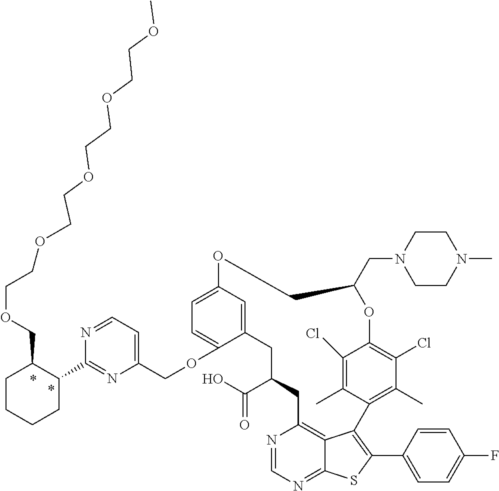 |
| 125 | 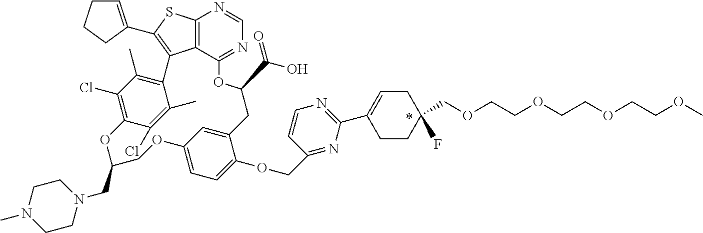 |
| 126 | 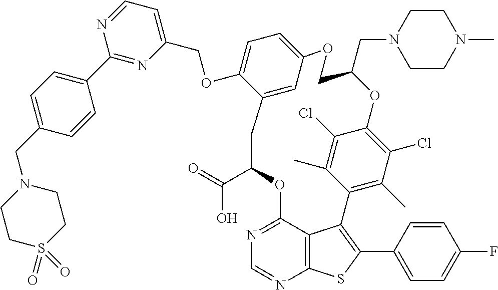 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(CN4CCS(=O)(=O)CC4)cc3)n1)OC[C@@H](CN1CCN(C)CC1)O2] |
| 127 | 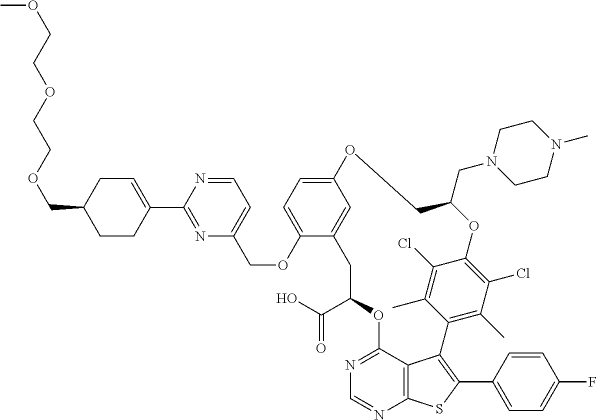 [SMILES: COCCOCCOC[C@H]1CC=C(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 128 | 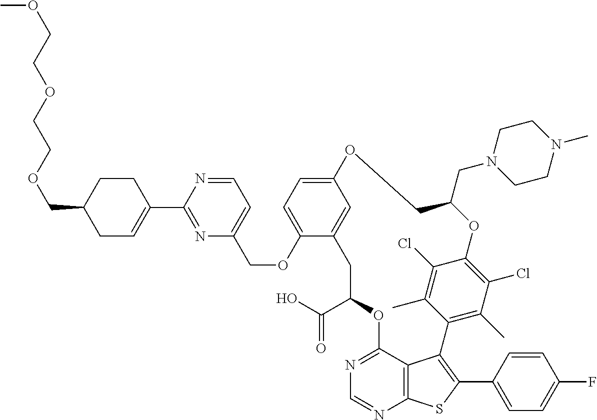 [SMILES: COCCOCCOC[C@@H]1CC=C(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 129 | 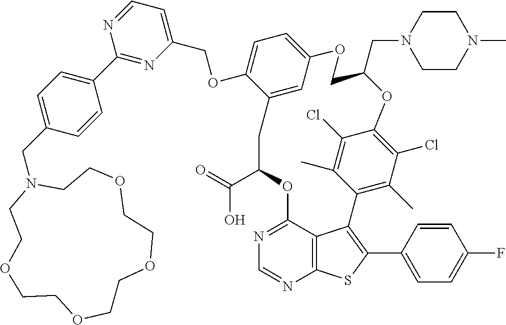 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(CN4CCOCCOCCOCCOCC4)cc3)n1)OC[C@@H](CN1CCN(C)CC1)O2] |
| 130 | 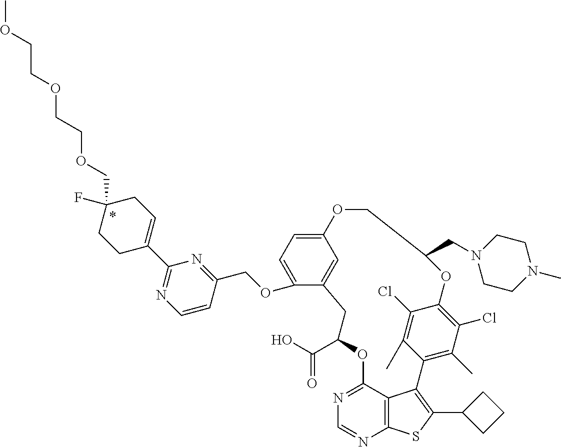 |
| 131 | 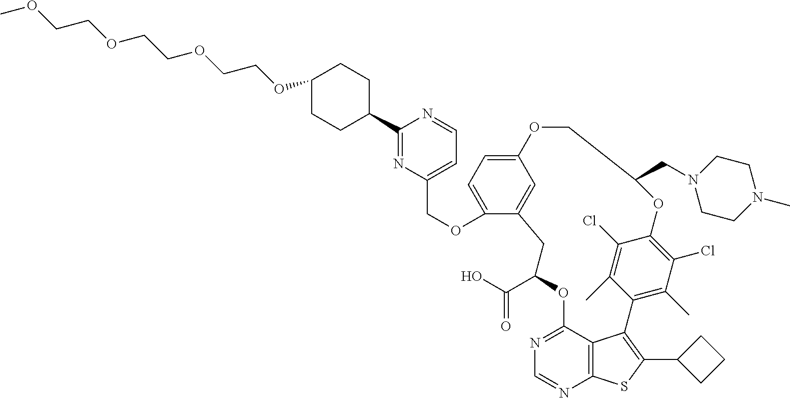 |
| 132 | 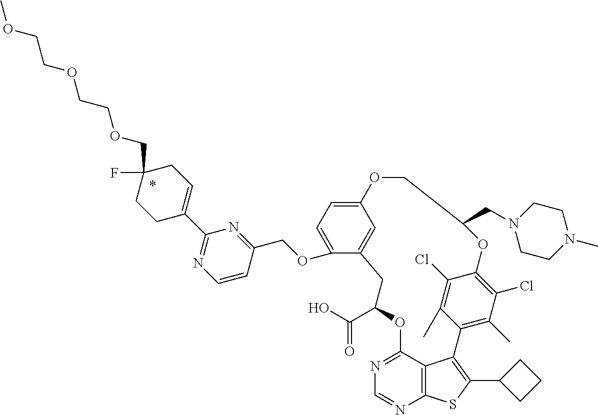 |
| 133 | 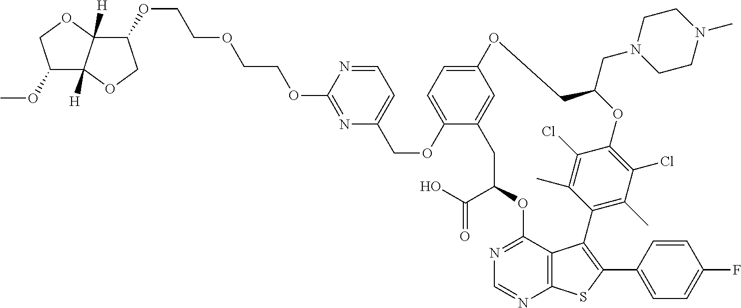 [SMILES: CO[C@@H]1CO[C@H]2[C@@H]1OC[C@H]2OCCOCCOc1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 134 | 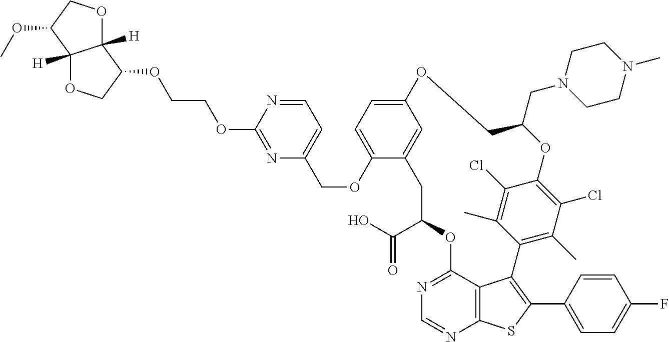 [SMILES: CO[C@@H]1CO[C@H]2[C@@H]1OC[C@H]2OCCOc1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 135 | 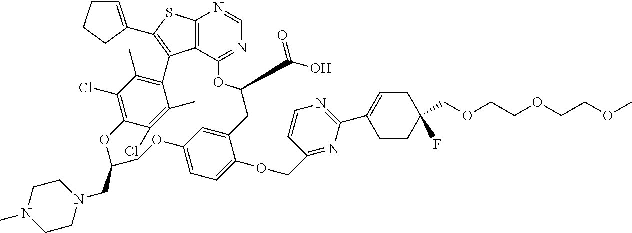 |
| 136 | 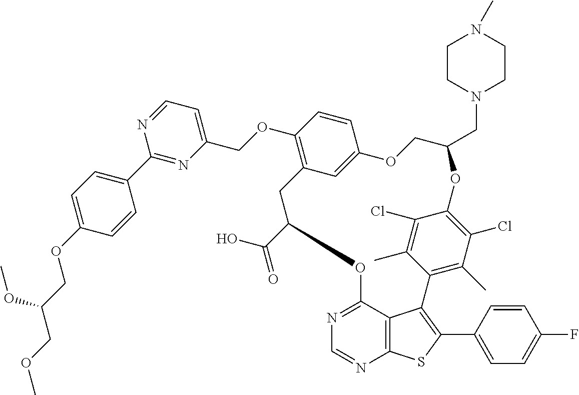 [SMILES: COC[C@@H](COc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1)OC] |
| 137 | 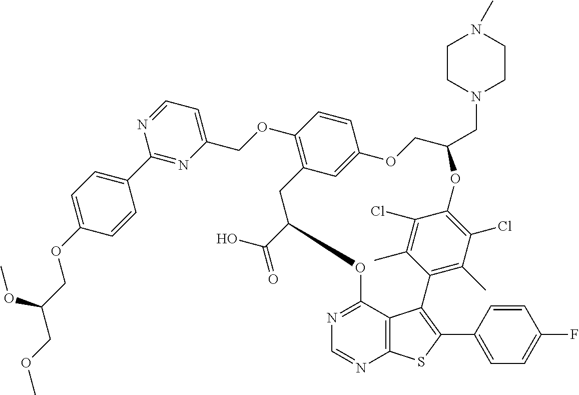 [SMILES: COC[C@H](COc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1)OC] |
| 138 | 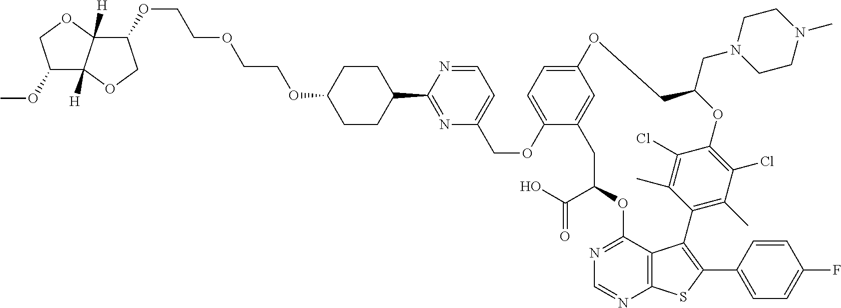 |
| 139 | 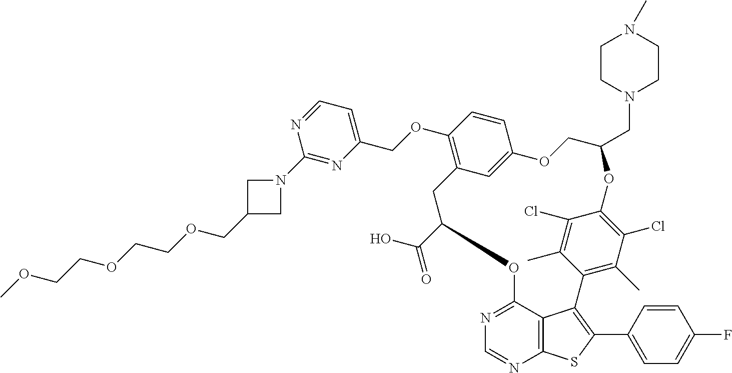 [SMILES: COCCOCCOCC1CN(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)C1] |
| 140 | 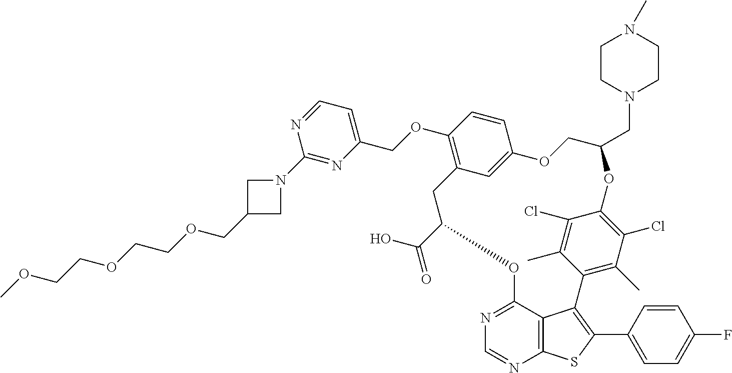 [SMILES: COCCOCCOCC1CN(c2nccc(COc3ccc4cc3C[C@@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)C1] |
| 141 | 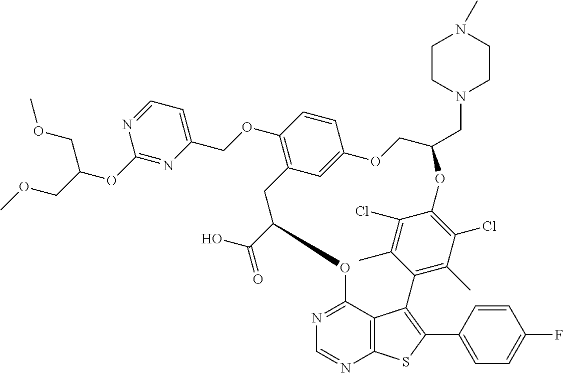 [SMILES: COCC(COC)Oc1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 142 | 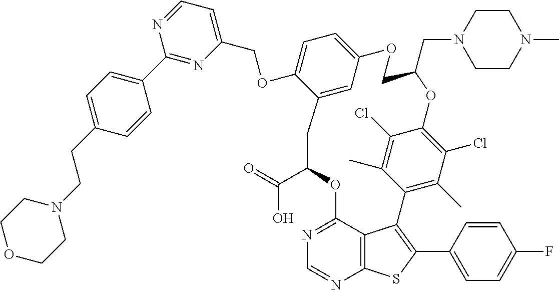 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(CCN4CCOCC4)cc3)n1)OC[C@@H](CN1CCN(C)CC1)O2] |
| 143 | 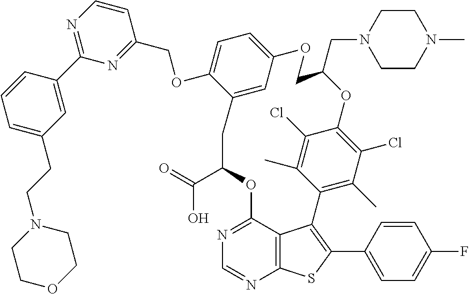 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3cccc(CCN4CCOCC4)c3)n1)OC[C@@H](CN1CCN(C)CC1)O2] |
| 144 | 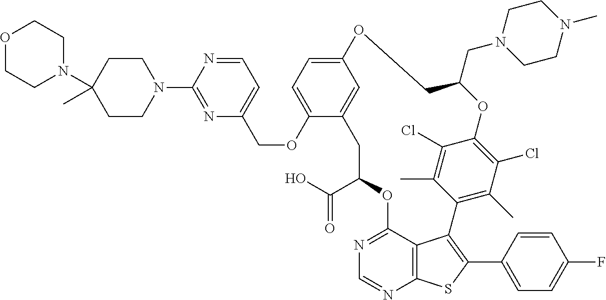 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CCC(C)(N4CCOCC4)CC3)n1)OC[C@@H](CN1CCN(C)CC1)O2] |
| 145 | 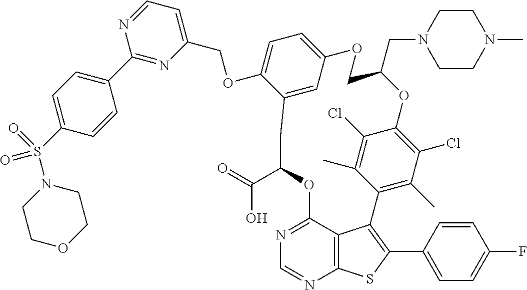 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(S(=O)(=O)N4CCOCC4)cc3)n1)OC[C@@H](CN1CCN(C)CC1)O2] |
| 146 | 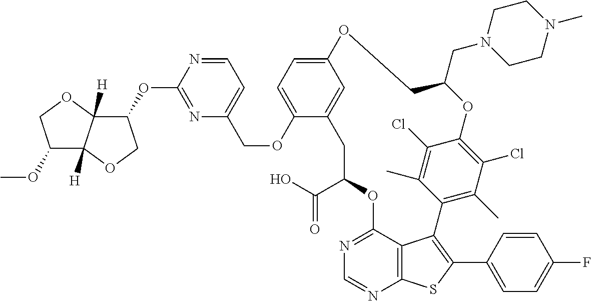 [SMILES: CO[C@@H]1CO[C@H]2[C@@H]1OC[C@H]2Oc1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 147 | 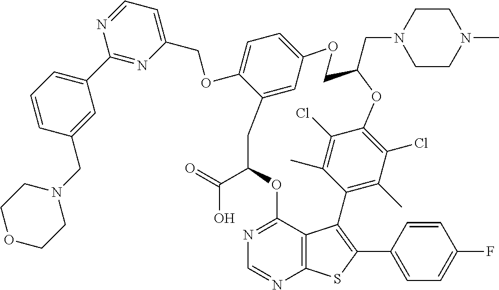 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3cccc(CN4CCOCC4)c3)n1)OC[C@@H](CN1CCN(C)CC1)O2] |
| 148 | 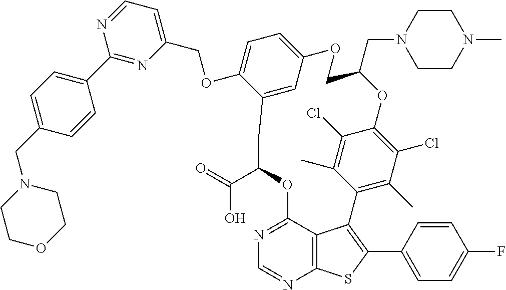 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(CN4CCOCC4)cc3)n1)OC[C@@H](CN1CCN(C)CC1)O2] |
| 149 | 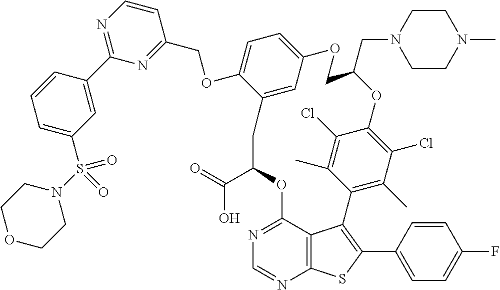 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3cccc(S(=O)(=O)N4CCOCC4)c3)n1)OC[C@@H](CN1CCN(C)CC1)O2] |
| 150 | 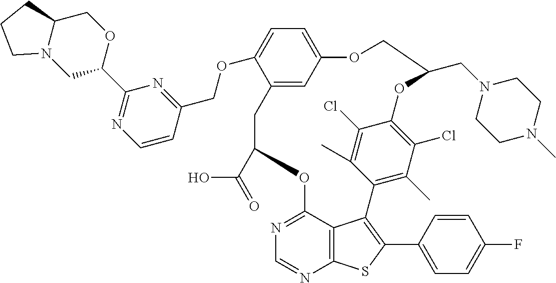 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc([C@@H]3CN4CCC[C@H]4CO3)n1)OC[C@@H](CN1CCN(C)CC1)O2] |
| 151 | 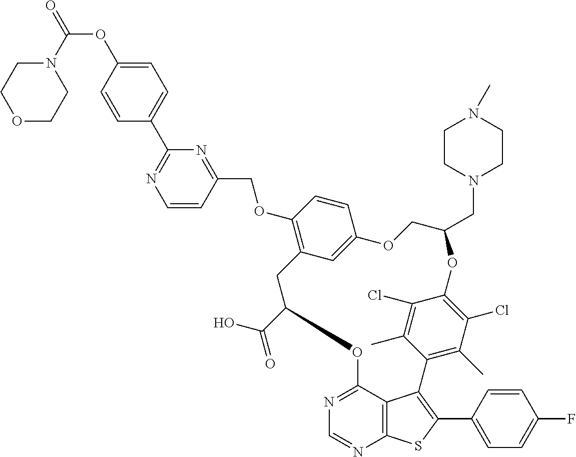 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OC(=O)N4CCOCC4)cc3)n1)OC[C@@H](CN1CCN(C)CC1)O2] |
| 152 | 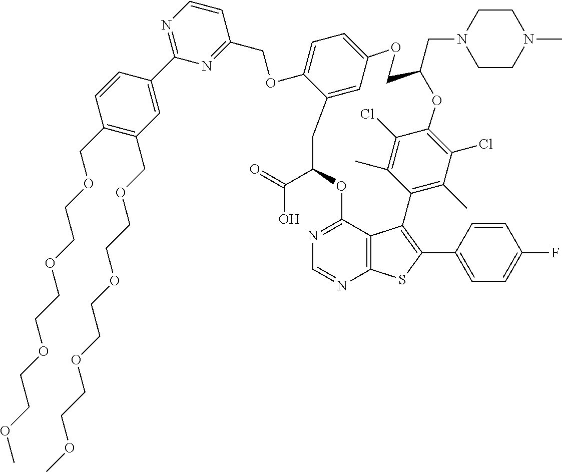 [SMILES: COCCOCCOCCOCc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1COCCOCCOCCOC] |
| 153 | 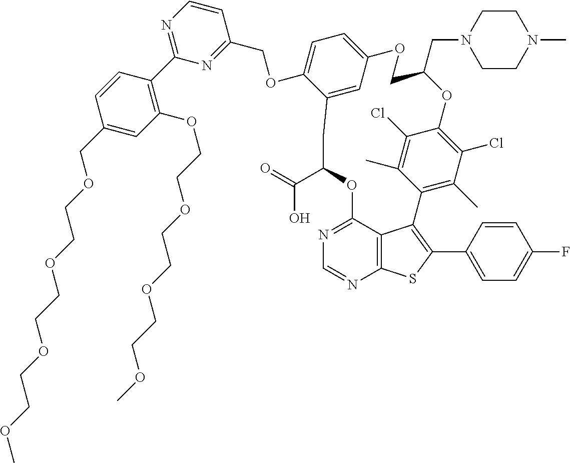 [SMILES: COCCOCCOCCOCc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)c(OCCOCCOCCOC)c1] |
| 154 | 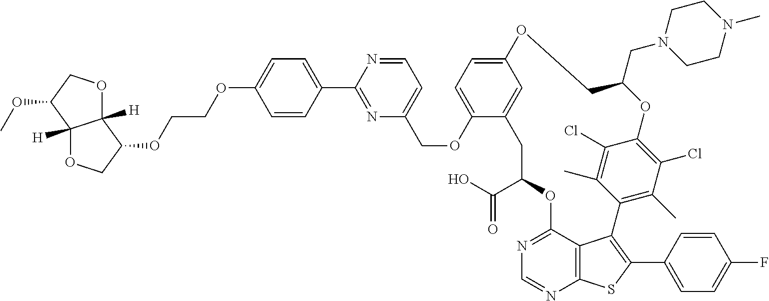 [SMILES: CO[C@@H]1CO[C@H]2[C@@H]1OC[C@H]2OCCOc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1] |
| 155 | 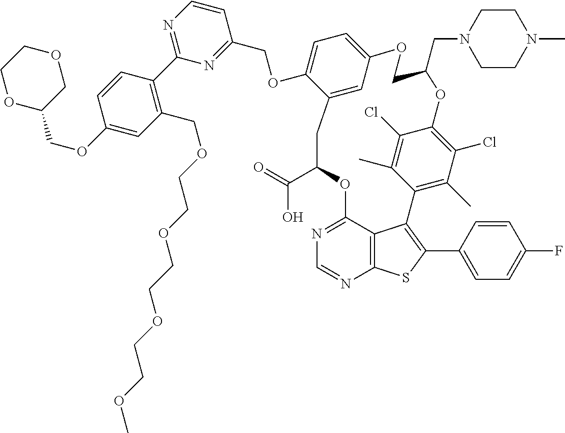 [SMILES: COCCOCCOCCOCc1cc(OC[C@H]2COCCO2)ccc1-c1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 156 | 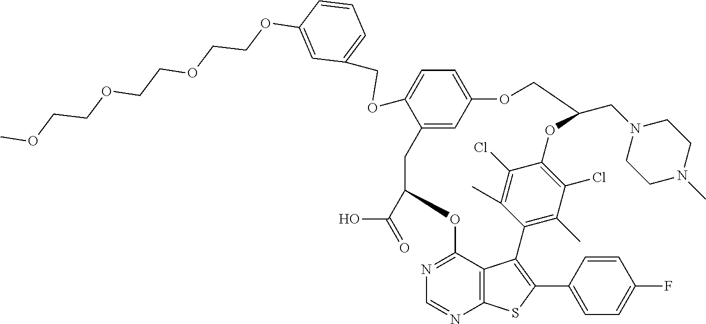 |
| 157 | 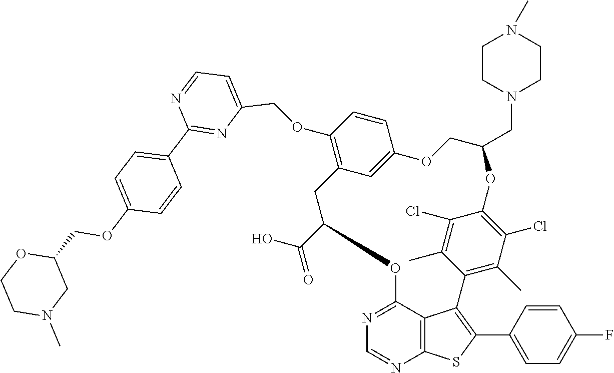 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OC[C@@H]4CN(C)CCO4)cc3)n1)OC[C@@H](CN1CCN(C)CC1)O2] |
| 158 | 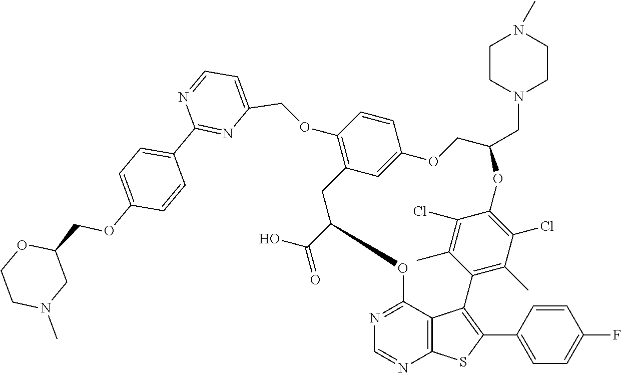 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OC[C@H]4CN(C)CCO4)cc3)n1)OC[C@@H](CN1CCN(C)CC1)O2] |
| 159 | 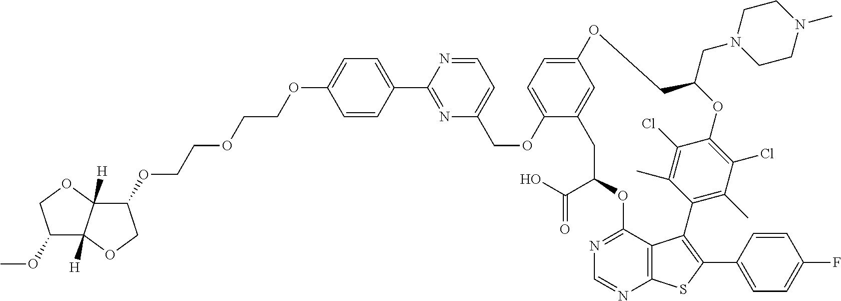 [SMILES: CO[C@@H]1CO[C@H]2[C@@H]1OC[C@H]2OCCOCCOc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1] |
| 160 | 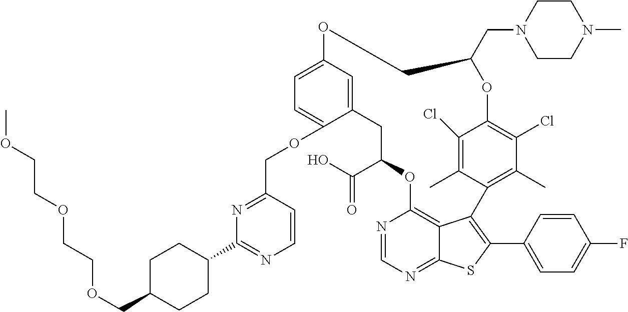 [SMILES: COCCOCCOC[C@H]1CC[C@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1] |
| 161 | 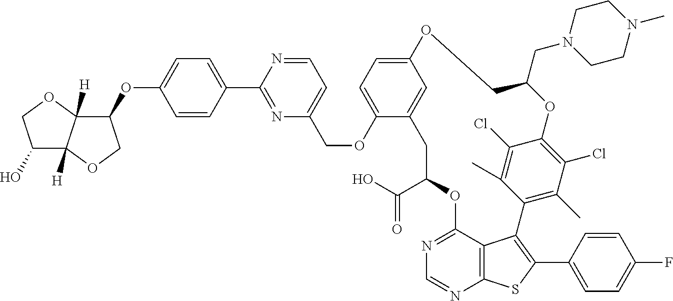 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(O[C@H]4CO[C@H]5[C@@H]4OC[C@H]5O)cc3)n1)OC[C@@H](CN1CCN(C)CC1)O2] |
| 162 | 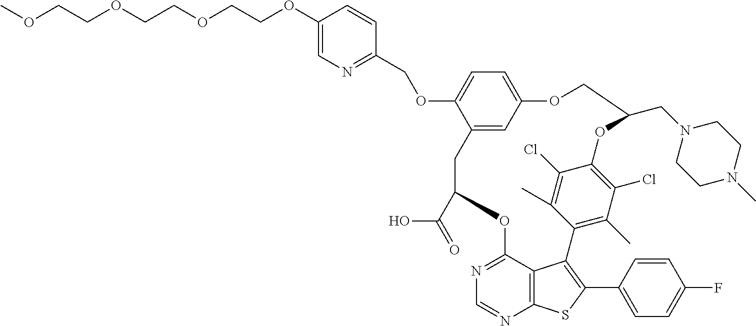 |
| 163 | 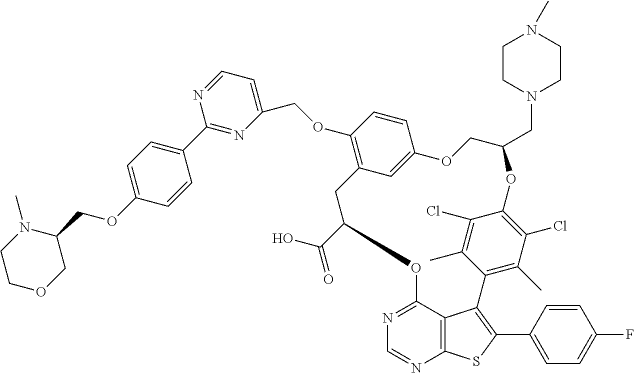 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OC[C@H]4COCCN4C)cc3)n1)OC[C@@H](CN1CCN(C)CC1)O2] |
| 164 | 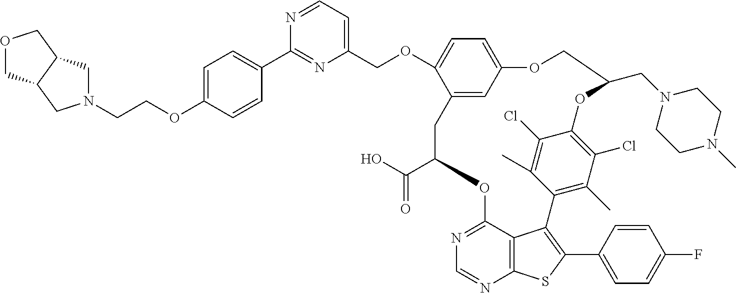 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OCCN4C[C@H]5COC[C@H]5C4)cc3)n1)OC[C@@H](CN1CCN(C)CC1)O2] |
| 165 | 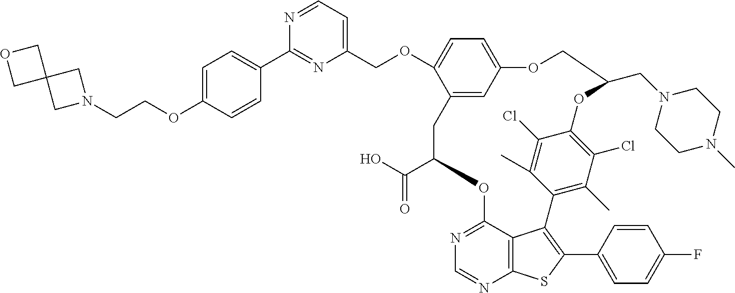 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OCCN4CC5(COC5)C4)cc3)n1)OC[C@@H](CN1CCN(C)CC1)O2] |
| 166 | 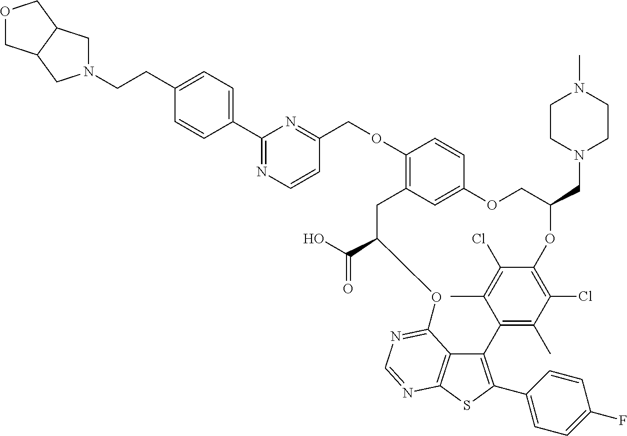 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(CCN4CC5COCC5C4)cc3)n1)OC[C@@H](CN1CCN(C)CC1)O2] |
| 167 | 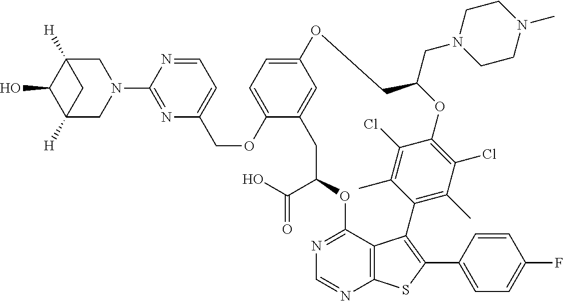 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3C[C@H]4C[C@@H](C3)[C@H]4O)n1)OC[C@@H](CN1CCN(C)CC1)O2] |
| 168 | 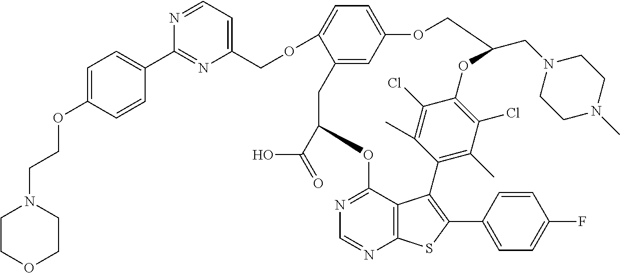 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OCCN4CCOCC4)cc3)n1)OC[C@@H](CN1CCN(C)CC1)O2] |
| 169 | 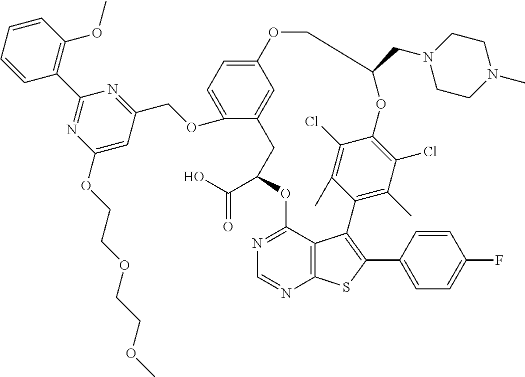 [SMILES: COCCOCCOc1cc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)nc(-c2ccccc2OC)n1] |
| 170 | 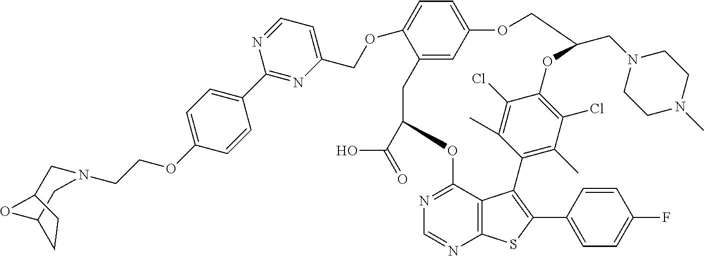 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OCCN4CC5CCC(C4)O5)cc3)n1)OC[C@@H](CN1CCN(C)CC1)O2] |
| 171 | 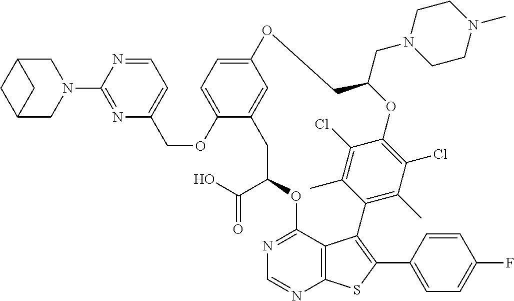 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3CC4CC(C4)C3)n1)OC[C@@H](CN1CCN(C)CC1)O2] |
| 172 | 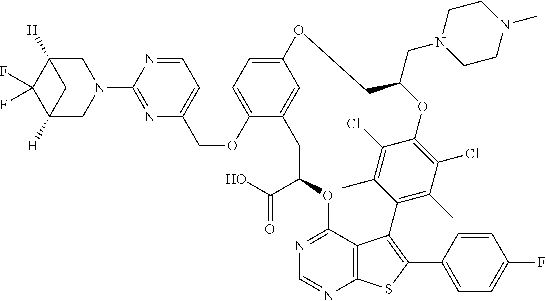 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(N3C[C@H]4C[C@@H](C3)C4(F)F)n1)OC[C@@H](CN1CCN(C)CC1)O2] |
| 173 | 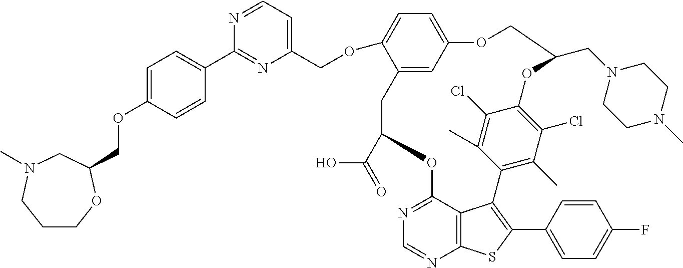 |
| 174 | 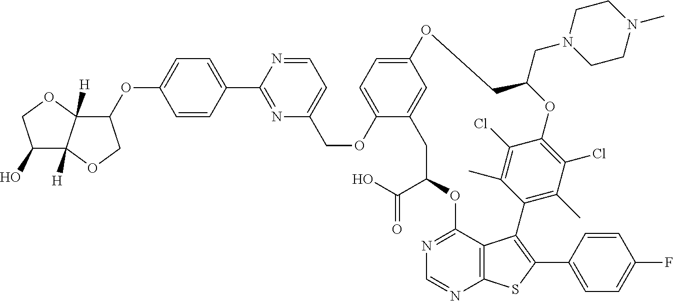 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(OC4CO[C@H]5[C@@H]4OC[C@@H]5O)cc3)n1)OC[C@@H](CN1CCN(C)CC1)O2] |
| 175 | 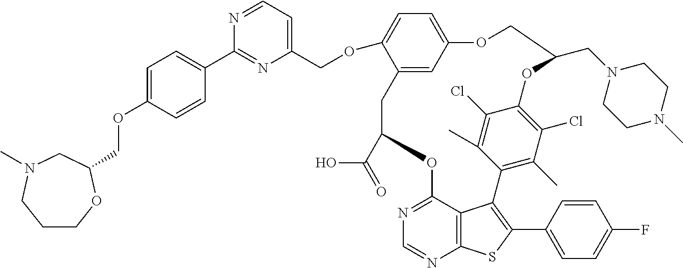 |
| 176 | 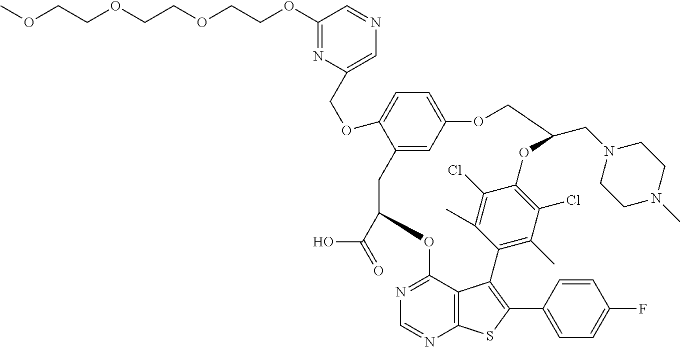 |
| 177 | 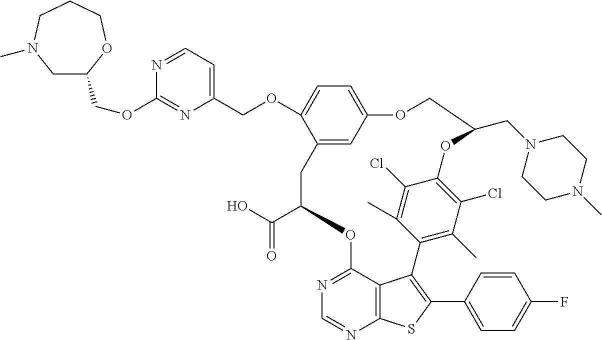 |
| 178 | 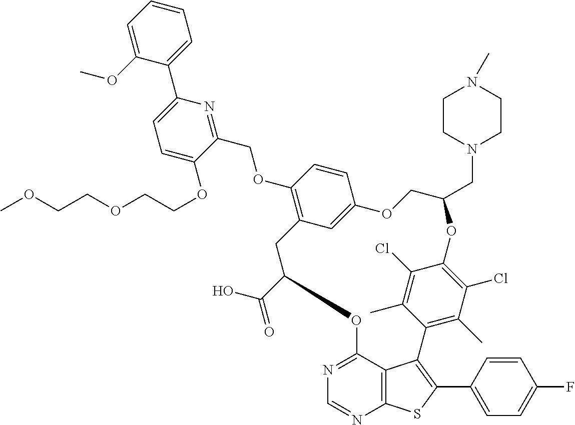 [SMILES: COCCOCCOc1ccc(-c2ccccc2OC)nc1COc1ccc2cc1C[C@H](C(=O)O)Oc1ncnc3sc(-c4ccc(F)cc4)c(c13)-c1c(C)c(Cl)c(c(Cl)c1C)O[C@H](CN1CCN(C)CC1)CO2] |

One embodiment pertains to Example 1, and pharmaceutically acceptable salts thereof:

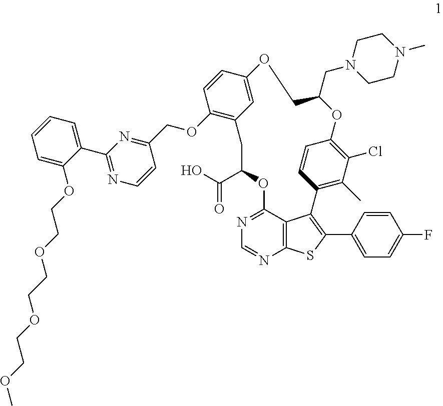

That is, in embodiments, the compound of Formula (I) is (7R,16R,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 15, and pharmaceutically acceptable salts thereof:

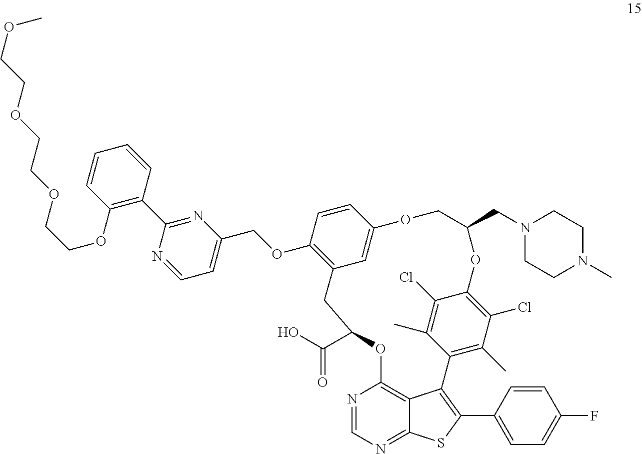 [SMILES: COCCOCCOCCOc1ccccc1-c1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1]

That is, in embodiments, the compound of Formula (I) is (7R,16R,21S)-19,23-dichloro-1-(4-fluorophenyl)-10-{[2-(2-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 16, and pharmaceutically acceptable salts thereof:

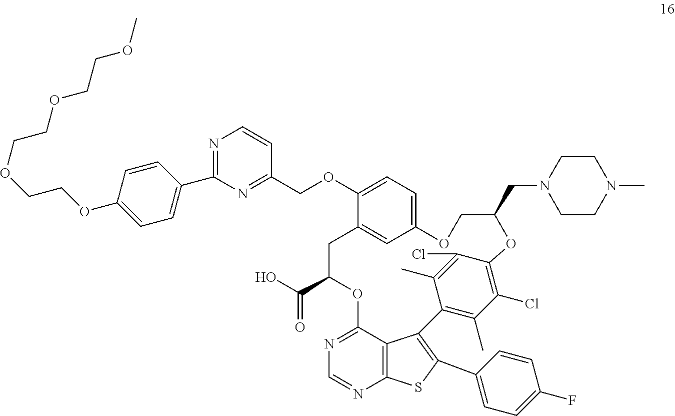 [SMILES: COCCOCCOCCOc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1]

That is, in embodiments, the compound of Formula (I) is (7R,16R)-19,23-dichloro-1-(4-fluorophenyl)-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yi)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 45, and pharmaceutically acceptable salts thereof:

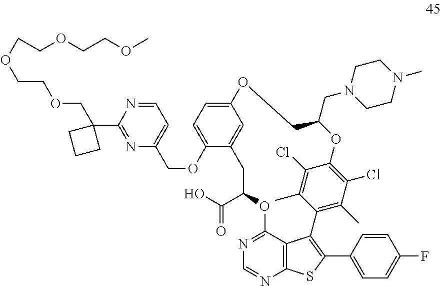 [SMILES: COCCOCCOCCOCC1(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CCC1]

That is, in embodiments, the compound of Formula (I) is (7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 86, and pharmaceutically acceptable salts thereof:

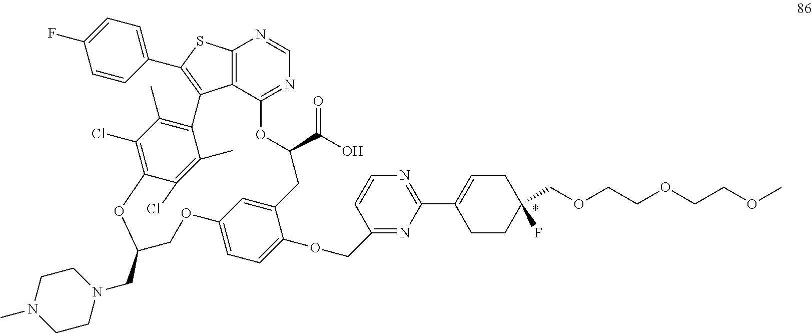

That is, in embodiments, the compound of Formula (I) is (7R,16R)-19,23-dichloro-10-({2-[(4S*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 87, and pharmaceutically acceptable salts thereof:

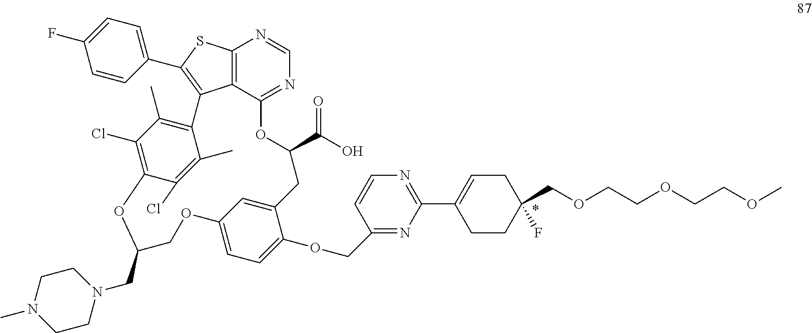

That is, in embodiments, the compound of Formula (I) is (7R,16R)-19,23-dichloro-10-({2-[(4R*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 127, and pharmaceutically acceptable salts thereof:

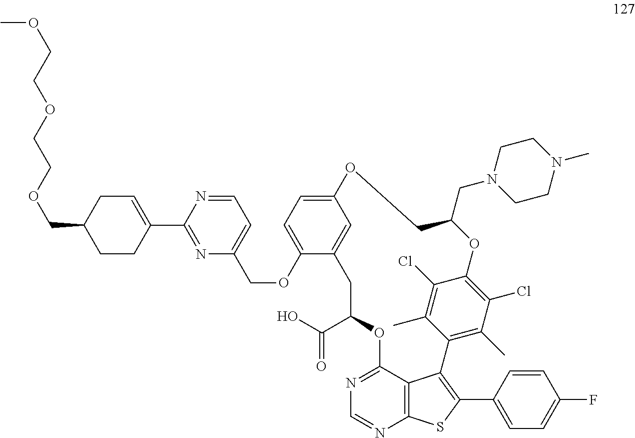 [SMILES: COCCOCCOC[C@H]1CC=C(c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1]

That is, in embodiments, the compound of Formula (I) is (7R,16R)-19,23-dichloro-1-(4-fluorophenyl)-10-({2-[(4R)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 136, and pharmaceutically acceptable salts thereof:

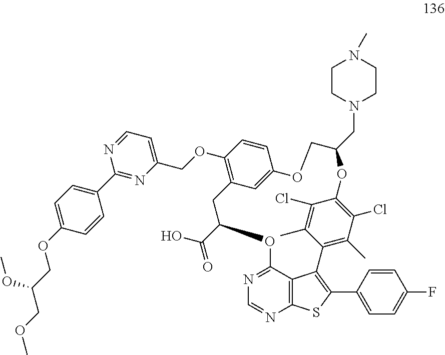 [SMILES: COC[C@@H](COc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1)OC]

That is, in embodiments, the compound of Formula (I) is (7R,16R)-19,23-dichloro-10-[(2-{4-[(25)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 137, and pharmaceutically acceptable salts thereof:

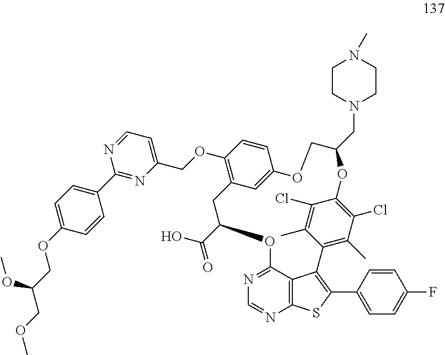 [SMILES: COC[C@H](COc1ccc(-c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)cc1)OC]

That is, in embodiments, the compound of Formula (I) is (7R,16R)-19,23-dichloro-10-[(2-{4-[(2R)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid, or pharmaceutically acceptable salts thereof.

Compounds of Formula (I) may be used in the form of pharmaceutically acceptable salts. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts have been described in S. M. Berge et al. J. Pharmaceutical Sciences, 1977, 66:1-19.

Compounds of Formula (I) may contain either a basic or an acidic functionality, or both, and may be converted to a pharmaceutically acceptable salt, when desired, by using a suitable acid or base. The salts may be prepared in situ during the final isolation and purification of the compounds of the disclosure.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts may be prepared in situ during the final isolation and purification of compounds of this disclosure by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

### Synthesis

The compounds described herein, including compounds of general Formula (I) and specific examples, may be prepared, for example, through the reaction routes depicted in schemes 1-9. The variables A², A³, A⁴, A⁶, A⁷, A⁸, A¹⁵, R^{A}, R⁵, R⁹, R^{10A}, R^{10B}, R¹, R¹², R¹³, R¹⁴, R¹⁶, W, X, and Y used in the following schemes have the meanings as set forth in the Summary and Detailed Description sections unless otherwise noted.

Abbreviations that may be used in the descriptions of the schemes and the specific examples have the meanings listed in the table below.

| Abbreviation | Definition |
|---|---|
| μL | microliter |
| Boc | tert-butoxycarbonyl |
| br s | broad singlet |
| d | duplet |
| DCI | desorption chemical ionization |
| DCM | dichloromethane |
| dd | double duplet |
| DIEA | N,N-Diisopropylethylamine |
| DMAP | dimethylaminopyridine |
| DMF | N,N-dimethylformamide |
| DMSO | dimethyl sulfoxide |
| eq or equiv | equivalents |
| ESI | electrospray ionization |
| Et | ethyl |
| g | gram |
| h | hours |
| HATU | 1-[bis(dimethylamino)methylene]- |
| | 1H-1,2,3-triazolo[4,5-b]pyridinium |
| | 3-oxid hexafluorophosphate |
| HOBt | 1-hydroxybenzotriazole hydrate |
| HPLC | high performance liquid chromatography |
| | or high pressure liquid chromatography |
| kg | kilogram |
| LC/MS or LCMS | liquid chromatography-mass spectrometry |
| m | multiplet |
| Me | methyl |
| MeOH | methanol |
| mg | milligram |
| min | minute |
| mL | milliliter |
| mmol | millimoles |
| MPLC | medium pressure liquid chromatography |
| MS | mass spectrum |
| NMP | N-methylpyrrolidone |
| NMR | nuclear magnetic resonance |
| Ph | phenyl |
| ppm | parts per million |
| psi | pounds per square inch |
| s | singlet |
| SFC | supercritical fluid chromatography |
| tBuOH or t-BuOH | tert-butanol |
| TFA | trifluoroacetic acid |
| THF | tetrahydrofuran |
| TLC | thin layer chromatography |
| XPhos | 2-dicyclohexylphosphino-2′,4′,6′- |
| | triisopropylbiphenyl |

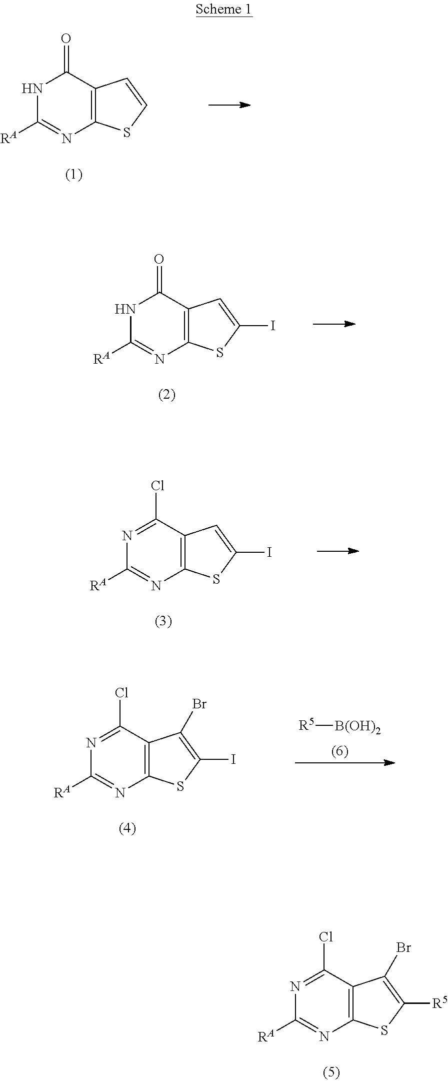

The synthesis of thienopyrimidine intermediates of formula (5) is described in Scheme 1. Thieno[2,3-d]pyrimidine-4(3H)-ones of formula (1), wherein R^{A} is as described herein, can be treated with periodic acid and iodine to provide 6-iodothieno[2,3-d]pyrimidin-4(3H)-ones of formula (2). The reaction is typically performed at an elevated temperature, for example from 60° C. to 70° C., in a solvent system such as, but not limited to, acetic acid, sulfuric acid and water, 4-Chloro-6-iodothieno[2,3-d]pyrimidines of formula (3) can be prepared by treating 6-iodothieno[2,3-d]pyrimidin-4(3H)-ones of formula (2) with phosphorous oxychloride. The reaction is typically carried out in a solvent such as, but not limited to, N,N-dimethylaniline at an elevated temperature, 5-Bromo-4-chloro-6-iodothieno[2,3-d]pyrimidines of formula (4) can be prepared by the treatment of 4-chloro-6-iodothieno[2,3-d]pyrimidines of formula (3) with N-bromosuccinimide in the presence of tetrafluoroboric acid-dimethyl ether complex. The reaction is typically performed at ambient temperature in a solvent such as, but not limited to, acetonitrile. Compounds of formula (5) can be prepared by reacting 5-bromo-4-chloro-6-iodothieno[2,3-d]pyrimidines of formula (4) with a boronic acid (or the equivalent boronate ester) of formula (6), wherein R⁵ is G³ as described herein, under Suzuki Coupling conditions described herein, known to those skilled in the art, or widely available in the literature.

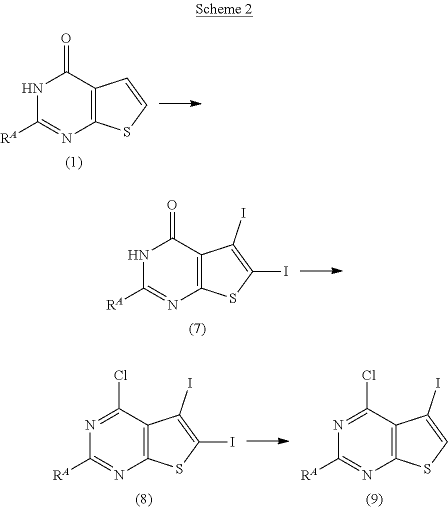

The synthesis of thienopyrimidine intermediates of formula (9) is described in Scheme 2. Thieno[2,3-d]pyrimidine-4(3H)-ones of formula (1), wherein R^{A} is as described herein, can be treated with periodic acid and iodine to provide 5,6-diiodothieno[2,3-d]pyrimidin-4(3H)-ones of formula (7). The reaction is typically performed at an elevated temperature, for example from 60° C. to 100° C., in a solvent system such as, but not limited to, acetic acid, sulfuric acid and water, 4-Chloro-5,6-diiodothieno[2,3-d]pyrimidines of formula (8) can be prepared by treating 5,6-diiodothieno[2,3-d]pyrimidin-4(3H)-ones of formula (7) with phosphorous oxychloride. The reaction is typically carried out in a solvent such as, but not limited to, N,N-dimethylaniline at an elevated temperature, 4-Chloro-5,6-diiodothieno[2,3-d]pyrimidines of formula (8) can be treated with tert-butylmagnesium chloride to provide compounds of formula (9). The reaction is typically performed at a low temperature in a solvent, such as, but not limited to, tetrahydrofuran.

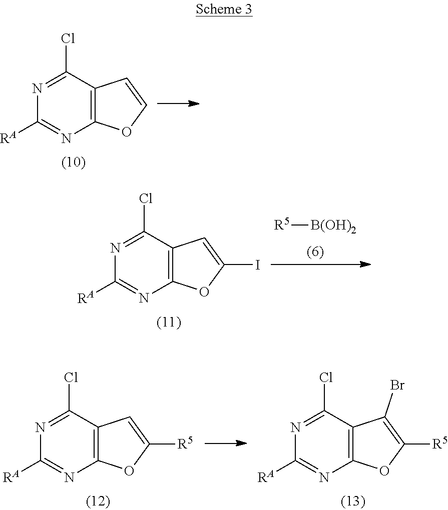

Scheme 3 describes the synthesis of furanopyrimidine intermediates of formula (13), 4-Chlorofuro[2,3-d]pyrimidines (10), wherein R^{A} is as described herein, can be treated with lithium diisopropylamide followed by iodine, in a solvent such as, but not limited to, tetrahydrofuran, to provide 4-chloro-6-iodofuro[2,3-d]pyrimidines of formula (11). The reaction is typically performed by first incubating a compound of formula (10) with lithium diisopropylamide at a low temperature, such as −78° C., followed by the addition of iodine and subsequent warming to ambient temperature. Compounds of formula (12) can be prepared by reacting 4-chloro-6-iodofuro[2,3-d]pyrimidines of formula (11) with a boronic acid (or the equivalent boronate ester) of formula (6) under Suzuki Coupling conditions described herein, known to those skilled in the art, or widely available in the literature. Compounds of formula (12) can be treated with N-bromosuccinimide to provide compounds of formula (13). The reaction is typically performed at ambient temperature in a solvent, such as, but not limited to, N,N-dimethylformamide.

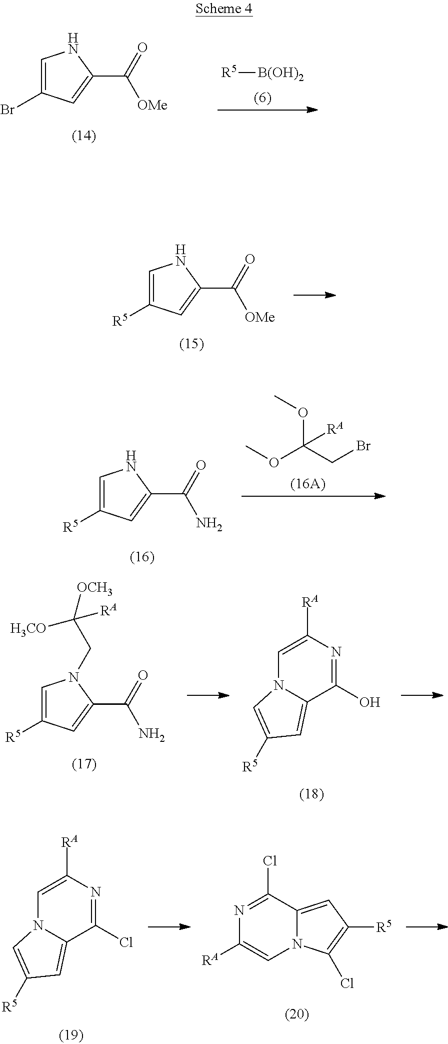

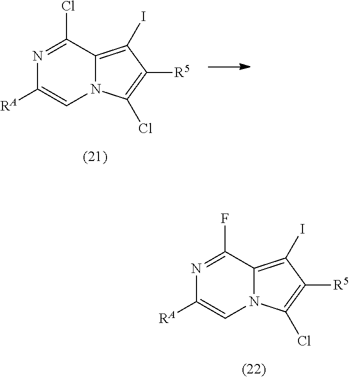

Scheme 4 describes the synthesis of pyrrolopyrazine intermediates of the formula (22), wherein R^{A} and R⁵ are as described herein. Compounds of the formula (15) can be prepared by reacting methyl 4-bromo-1H-pyrrole-2-carboxylate (14) with a boronic acid (or the equivalent boronate ester) of formula (6) under Suzuki Coupling conditions described herein, known to those skilled in the art, or widely available in the literature. Compounds of formula (15) can be heated in the presence of an aqueous ammonium hydroxide solution to provide compounds of formula (16). Compounds of the formula (17) can be prepared by treatment of pyrroles of formula (16) with 2-bromo-1,1-dimethoxyethane in the presence of a base such as, but not limited to, cesium carbonate. The reaction is typically performed in a solvent such as, but not limited to, N,N-dimethylformamide at elevated temperatures ranging from 80° C. to 90° C. Compounds of formula (17) can be treated with hydrogen chloride in a solvent such as, but not limited to, dichloromethane to provide compounds of the formula (18). Compounds of the formula (19) can be prepared by reacting intermediates (18) with phosphorous oxychloride in the presence of a base such as, but not limited to, N,N-diisopropylethylamine. The reaction is typically performed at elevated temperatures such as ranging from 100° C. to 115° C. Compounds of formula (19) can be treated with N-chlorosuccinimide in a solvent system such as, but not limited to, tetrahydrofuran to provide compounds of formula (20). The reaction is typically performed at an elevated temperature. Compounds of formula (21) can be prepared by reacting compounds of formula (20) with N-iodosuccinimide at an elevated temperature in a solvent such as, but not limited to, N,N-dimethylformamide. Compounds of formula (21) can be treated with tetramethylammonium fluoride to provide compounds of formula (22). The reaction is typically performed at ambient temperature in a solvent such as, but not limited to, N,N-dimethylformamide.

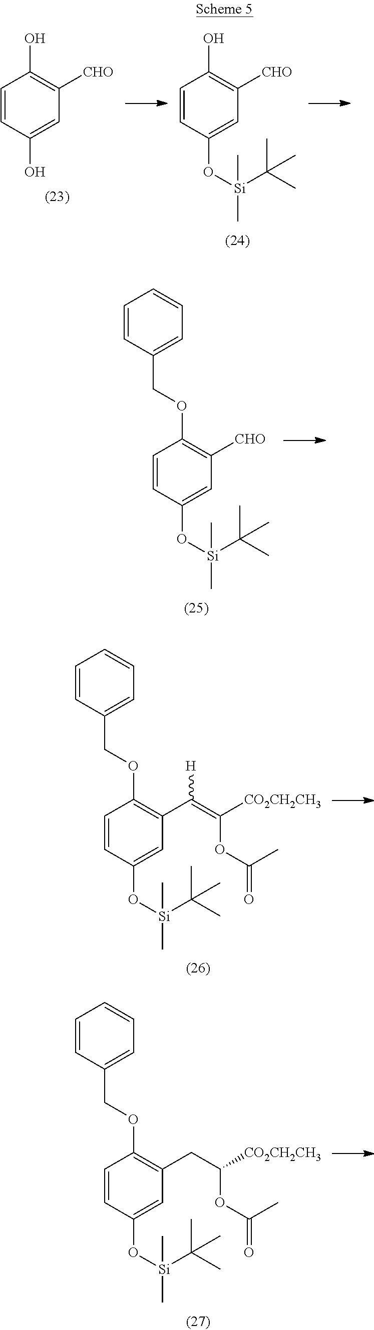

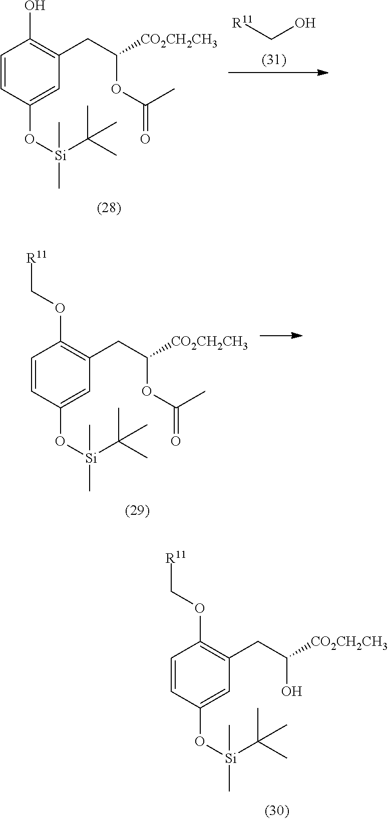

Scheme 5 describes the synthesis of propanoate intermediates of formula (30), 2,5-Dihydroxybenzaldehyde (23) can be treated with tert-butylchlorodimethylsilane to provide mono-silylated intermediate (24). The reaction is typically conducted at ambient temperature in the presence of a base such as, but not limited to, imidazole in a solvent such as, but not limited to, dichloromethane. The mono-silylated intermediate can be reacted with benzyl bromide to provide 2-(benzyloxy)-5-((tert-butyldimethylsilyl)oxy)benzaldehyde (25). The reaction is typically performed in the presence of a base such as, but not limited to, potassium carbonate, and in a solvent such as, but not limited to acetone, N,N-dimethylformamide, or mixtures thereof. The reaction is typically initiated at room temperature followed by heating to an elevated temperature, 2-(Benzyloxy)-5-((tert-butyldimethylsilyl)oxy)benzaldehyde (25) can be treated with ethyl 2-acetoxy-2-(diethoxyphosphoryl)acetate to provide (E)/(Z)-ethyl 2-acetoxy-3-(2-(benzyloxy)-5-((tert-butyldimethylsilyl)oxy)phenyl)acrylates (26). The reaction is typically run in the presence a base such as, but not limited to, cesium carbonate in a solvent such as, but not limited to, tetrahydrofuran, toluene, or mixtures thereof. (E)/(Z)-Ethyl 2-acetoxy-3-(2-(benzyloxy)-5-((tert-butyldimethylsilyl)oxy)phenyl)acrylates (26) can be reacted with the catalyst (R,R)-Rh EtDuPhos (1,2-bis[(2R,5R)-2,5-diethylphospholano]benzene(1,5-cyclooctadiene)rhodium (I) trifluoromethanesulfonate) under an atmosphere of hydrogen gas in a solvent such as, but not limited to, methanol, to provide (R)-ethyl 2-acetoxy-3-(2-(benzyloxy)-5-((tert-butyldimethylsilyl)oxy)phenyl)propanoate (27). The reaction is typically performed at 35° C. under 50 psi of hydrogen gas. Ethyl (R)-2-acetoxy-3-(5-((tert-butyldimethylsilyl)oxy)-2-hydroxyphenyl)propanoate (28) can be provided by reacting (R)-ethyl 2-acetoxy-3-(2-(benzyloxy)-5-((tert-butyldimethylsilyl)oxy)phenyl)propanoate (27) under hydrogenolysis conditions, such as in the presence of 5% palladium on carbon under 50 psi of hydrogen gas in a solvent such as, but not limited to, ethanol at an elevated temperature, such as, but not limited to, 35° C. Ethyl (R)-2-acetoxy-3-(5-((tert-butyldimethylsilyl)oxy)-2-hydroxyphenyl)propanoate (28) can be reacted with compounds of formula (31), wherein R¹¹ is as described herein, under Mitsunobu conditions described herein, known to those skilled in the art, or widely available in the literature, to provide compounds of formula (29). Compounds of the formula (29) can be treated with ethanol in the presence of a base such as, but not limited to, potassium carbonate or sodium ethoxide, to provide compounds of the formula (30).

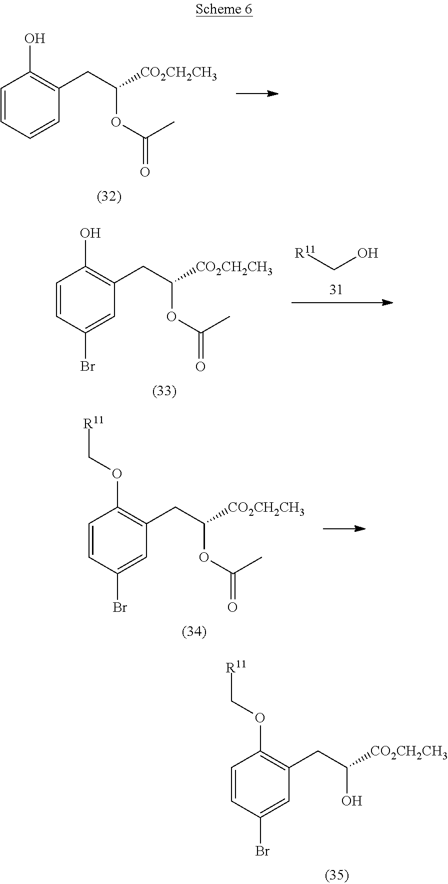

Scheme 6 describes the synthesis of propanoate intermediates of formula (35). (R)-Ethyl 2-acetoxy-3-(2-hydroxyphenyl)propanoate (32), which can be prepared using methods similar to those described for compounds of formula (28) in Scheme 5 or using methods described herein, can be treated with a brominating agent such as N-bromosuccinimide to provide (R)-ethyl 2-acetoxy-3-(5-bromo-2-hydroxyphenyl)propanoate (33). The reaction is typically performed in a solvent such as, but not limited to, tetrahydrofuran, at a low temperature, such as −30° C. to 0° C., before warming to ambient temperature. (R)-Ethyl 2-acetoxy-3-(5-bromo-2-hydroxyphenyl)propanoate (33) can be reacted with compounds of formula (31), wherein R¹¹ is as described herein, under Mitsunobu conditions described herein or in the literature to provide compounds of formula (34). Compounds of formula (34) can be treated with ethanol in the presence of a base such as, but not limited to, potassium carbonate or sodium ethoxide at ambient temperature to provide compounds of formula (35).

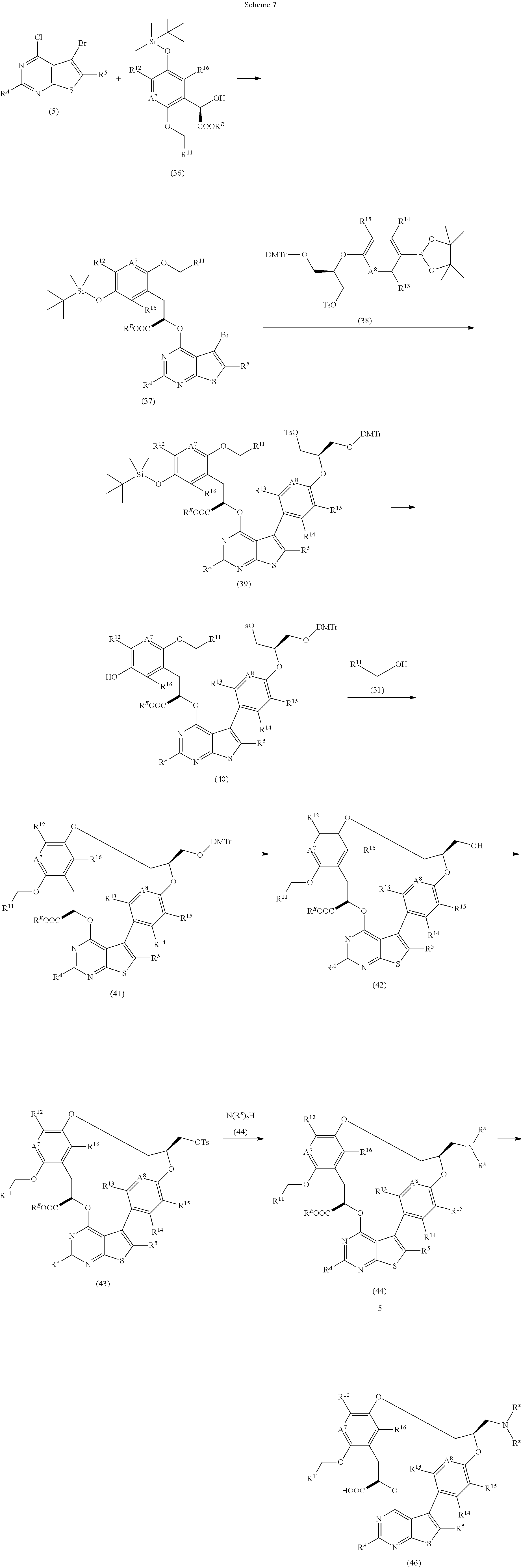

Scheme 7 describes the synthesis of macrocyclic compounds of the formula (46), which are representative of compounds of Formula (I). Intermediates of the formula (5) can be reacted with compounds of the formula (36), wherein A⁷, R¹¹, R¹², R¹⁶ are as described herein and R^{E} is alkyl, in the presence of base such as, but not limited to, cesium carbonate, to provide compounds of the formula (37). The reaction is typically conducted at an elevated temperature, such as, but not limited to 65° C., in a solvent such as but not limited to tert-butanol, N,N-dimethylformamide, or mixtures thereof. Compounds of formula (39) can be prepared by reacting compounds of formula (37) with a boronate ester (or the equivalent boronic acid) of formula (38) under Suzuki Coupling conditions described herein or in the literature. Compounds of formula (39) can be treated with tetrabutylammonium fluoride in a solvent system such as dichloromethane, tetrahydrofuran or mixtures thereof to provide compounds of formula (40). Treatment of compounds of formula (40) with a base such as, but not limited to, cesium carbonate in a solvent such as, but not limited to, N,N-dimethylformamide, will provide compounds of formula (41). The reaction is typically performed at an elevated temperature, or more preferably at ambient temperature. Compounds of the formula (41) can be deprotected to give compounds of the formula (42) using procedures described herein or available in the literature. For example, compounds of formula (41) can be treated with formic acid at ambient temperature in a solvent system such as, but not limited to, dichloromethane and methanol, to provide compounds of the formula (42). Compounds of the formula (42) can be treated with para-toluenesulfonyl chloride in the presence of a base such as, but not limited to, triethylamine or DABCO (1,4-diazabicyclo[2.2.2]octane) to provide compounds of formula (43). The reaction is typically performed at low temperature before warming to room temperature in a solvent such as, but not limited to, dichloromethane. Compounds of formula (43) can be reacted with amine nucleophiles of formula (44), wherein two RX, together with the nitrogen to which they are attached, optionally form a heterocycle, to provide intermediates of formula (45). The reaction is typically performed in a solvent such as, but not limited to, N,N-dimethylformamide, at ambient temperature before heating to 35° C. to 40° C. Compounds of formula (46) can be prepared by treating compounds of formula (45) with lithium hydroxide. The reaction is typically performed at ambient temperature in a solvent such as, but not limited to, tetrahydrofuran, methanol, water, or mixtures thereof.

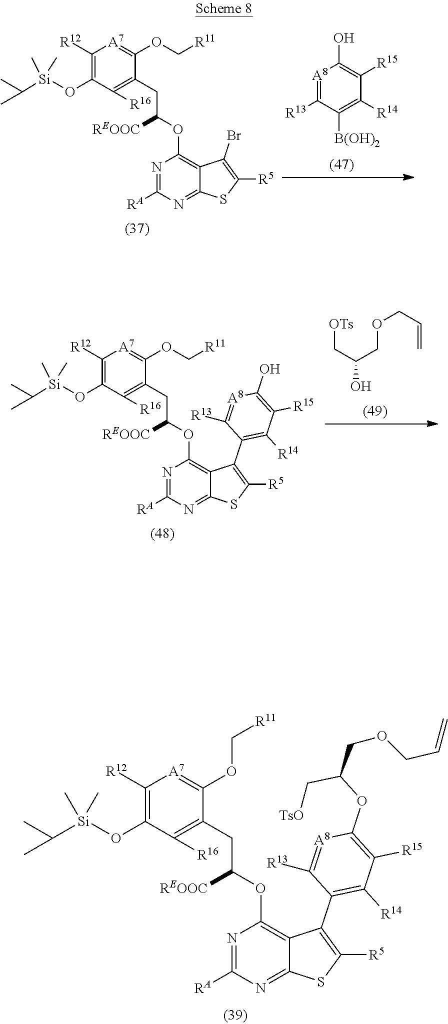

Scheme 8 describes an alternative synthesis of intermediates of the formula (39). Compounds of formula (48) can be prepared by reacting compounds of formula (37) with a boronate ester (or the equivalent boronic acid) of formula (47) under Suzuki Coupling conditions described herein or available in the literature. Compounds of the formula (48) can be reacted with compounds of formula (49) under Mitsunobu conditions described herein or available in the literature to provide compounds of the formula (39). Compounds of the formula (39) can be further treated as described in Scheme 7 or using methods described herein to provide macrocyclic compounds of the formula (46), which are representative of compounds of Formula (I).

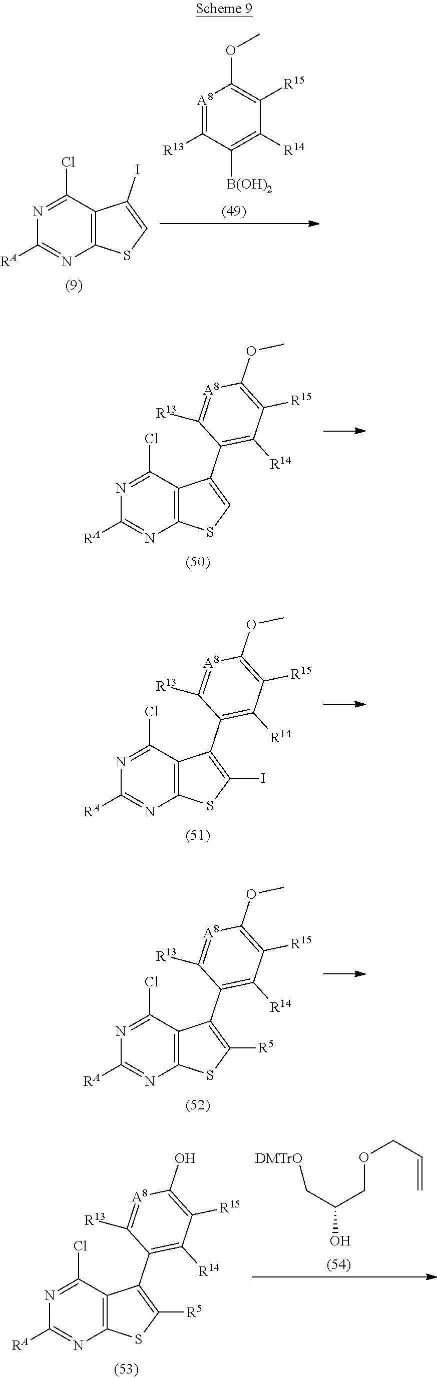

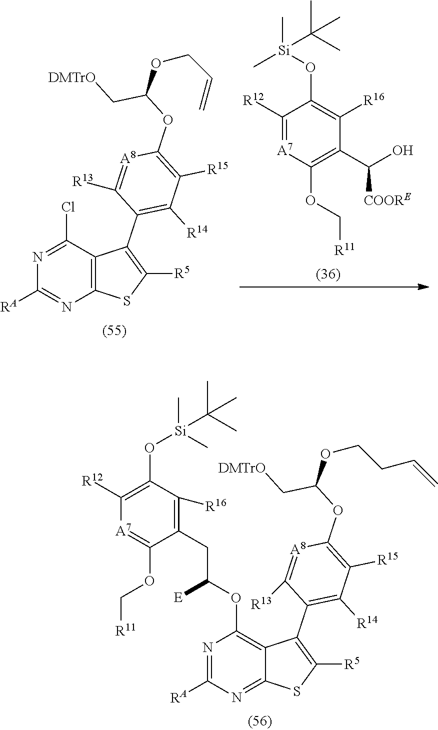

Scheme 9 describes the synthesis of compounds of formula (56). Compounds of formula (50) can be prepared by reacting compounds of formula (9) with a boronate ester (or the equivalent boronic acid) of formula (49) under Suzuki Coupling conditions described herein or available in the literature. Compounds of formula (50) can be treated with a strong base such as, but not limited to lithium diisopropylamide, followed by the addition of iodine to provide compounds of the formula (51). The reaction is typically performed in a solvent such as, but not limited to, tetrahydrofuran, at a reduced temperature before warming to ambient temperature. Compounds of formula (52) can be prepared by reacting compounds of formula (51) with a boronate ester (or the equivalent boronic acid) of formula (6) under Suzuki Coupling conditions described herein or known in the literature. Compounds of formula (52) can be treated with aluminum trichloride to provide compounds of formula (53). The reaction is typically performed at an elevated temperature, for example from 60° C. to 70° C., in a solvent, such as but not limited to, 1,2-dichloroethane. Compounds of formula (53) can be treated with compounds of formula (54) under Mitsunobu conditions described herein or available in the literature to provide compounds of the formula (55). Compounds of formula (55) can be reacted with compounds of formula (36) in the presence of a base such as, but not limited to, cesium carbonate to provide compounds of formula (56). The reaction is typically performed at an elevated temperature in a solvent such as tert-butanol, N,N-dimethylformamide, or mixtures thereof. Compounds of formula (56) can be used as described in subsequent steps herein to provide compounds of Formula (I).

It should be appreciated that the synthetic schemes and specific examples as illustrated in the synthetic examples section are illustrative and are not to be read as limiting the scope of the disclosure as it is defined in the appended claims. All alternatives, modifications, and equivalents of the synthetic methods and specific examples are included within the scope of the claims.

Optimum reaction conditions and reaction times for each individual step can vary depending on the particular reactants employed and substituents present in the reactants used. Specific procedures are provided in the Synthetic Examples section. Reactions can be worked up in the conventional manner. e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Manipulation of the reaction conditions, reagents and sequence of the synthetic route, protection of any chemical functionality that can not be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art: examples of which can be found in T. Greene and P. Wuts, Protecting Groups in Organic Synthesis (3^{rd} ed.). John Wiley & Sons, NY (1999), which is incorporated herein by reference in its entirety. Synthesis of the compounds of the disclosure can be accomplished by methods analogous to those described in the synthetic schemes described hereinabove and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be prepared by carrying out one of the above procedures using a pure geometric isomer as a starting material, or by resolution of a mixture of the geometric isomers of the compound or intermediates using a standard procedure such as chromatographic separation.

### Pharmaceutical Compositions

When employed as a pharmaceutical, a compound of the disclosure is typically administered in the form of a pharmaceutical composition. One embodiment pertains to a pharmaceutical composition comprising a therapeutically effective amount of a compound of Formula (I) according to claim 1, or a pharmaceutically acceptable salt thereof, in combination with a pharmaceutically acceptable carrier. The phrase “pharmaceutical composition” refers to a composition suitable for administration in medical or veterinary use.

The term “pharmaceutically acceptable carrier” as used herein, means a non-toxic, inert solid, semi-solid or liquid filler, diluent, encapsulating material or formulation auxiliary. Methods of Use

The compounds of Formula (I), or pharmaceutically acceptable salts thereof, and pharmaceutical compositions comprising a compound of Formula (I), or a pharmaceutically acceptable salt thereof, may be administered to a subject suffering from a disorder or condition associated with MCL-1 overexpression or up-regulation. The term “administering” refers to the method of contacting a compound with a subject. Disorders or conditions associated with MCL-1 overexpression or up-regulation may be treated prophylactically, acutely, and chronically using compounds of Formula (I), depending on the nature of the disorder or condition. Typically, the host or subject in each of these methods is human, although other mammals may also benefit from the administration of a compound of Formula (I).

A “MCL-1-mediated disorder or condition” is characterized by the participation of MCL-1 in the inception and/or manifestation of one or more symptoms or disease markers, maintenance, severity, or progression of a disorder or condition.

In embodiments, the present disclosure provides a method for treating multiple myeloma. The method comprises the step of administering to a subject in need thereof a therapeutically effective amount of a compound of Formula (I) or a preferred embodiment thereof, with or without a pharmaceutically acceptable carrier. In embodiments, the present disclosure provides compounds of the disclosure, or pharmaceutical compositions comprising a compound of the disclosure, for use in medicine. In a particular embodiment, the present disclosure provides compounds of the disclosure, or pharmaceutical compositions comprising a compound of the disclosure, for use in the treatment of diseases or disorders as described herein above.

One embodiment is directed to the use of a compound according to Formula (I), or a pharmaceutically acceptable salt thereof in the preparation of a medicament. The medicament optionally can comprise at least one additional therapeutic agent. In some embodiments the medicament is for use in the treatment of diseases and disorders as described herein above.

This disclosure is also directed to the use of a compound according to Formula (I), or a pharmaceutically acceptable salt thereof in the manufacture of a medicament for the treatment of the diseases and disorders as described herein above. The medicament optionally can comprise at least one additional therapeutic agent.

The compounds of Formula (I) may be administered as the sole active agent or it may be co-administered with other therapeutic agents, including other compounds that demonstrate the same or a similar therapeutic activity and that are determined to be safe and efficacious for such combined administration. The term “co-administered” means the administration of two or more different therapeutic agents or treatments (e.g., radiation treatment) that are administered to a subject in a single pharmaceutical composition or in separate pharmaceutical compositions. Thus co-administration involves administration at the same time of a single pharmaceutical composition comprising two or more different therapeutic agents or administration of two or more different compositions to the same subject at the same or different times.

### EXAMPLES

The following Examples may be used for illustrative purposes and should not be deemed to narrow the scope of the present disclosure.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference. Multiplicities were given as singlet (s), doublet (d), triplet (t), quartet (q), quintuplet (quin), multiplet (m) and broad (br).

### Example 1

### (7R,16R,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 1A

### 2-(benzyloxy)-5-((tert-butyldimethylsilyl)oxy)benzaldehyde

A 2 L round bottom flask was charged with 2,5-dihydroxybenzaldehyde (30 g), imidazole (29.6 g) and dichloromethane (543 mL). The flask was placed in a water bath and solid tert-butylchlorodimethylsilane (32.7 g) was added. The reaction mixture was stirred at ambient temperature for 15 minutes at which point thin-layer chromatography indicated complete consumption of starting material. The reaction mixture was poured into a separatory funnel with 200 mL water. The biphasic mixture was shaken and layers were separated. The aqueous layer was washed with 100 mL dichloromethane and the organic layers were combined. After drying over Na₂SO₄, filtration, and concentration, the crude material was used as such for the next step. A 1 L three-necked round bottom flask equipped with an internal temperature probe, a reflux condenser, and a stir bar was charged with 5-((tert-butyldimethylsilyl)oxy)-2-hydroxybenzaldehyde (45 g, 178 mmol) in acetone (297 mL). Solid K₂CO₃ (27.1 g) was added followed by dropwise addition of neat benzyl bromide (21.21 mL). The mixture was stirred at ambient temperature for 10 minutes and was heated to 55° C. The reaction was continued overnight. The reaction was cooled to ambient temperature and was poured over cold water (200 mL). The mixture was transferred to a 1 L separatory funnel. The crude product was extracted with ethyl acetate (3×250 mL). The combined organic layers were dried over Na₂SO₄, filtered, and concentrated. The crude material was purified by silica gel chromatography over a 330 g column on a Grace Reveleris system (0-5% ethyl acetate/heptanes elution gradient). Fractions containing the desired product were combined, concentrated and dried under vacuum to obtain the title compound. ¹H NMR (501 MHz, DMSO-d₆) δ ppm 10.35 (s, 1H), 7.51-7.47 (m, 2H), 7.42-7.37 (m, 2H), 7.35-7.31 (m, 1H), 7.22 (d, 1H), 7.15 (dd, 1H), 7.11 (d, 1H), 5.21 (s, 2H), 0.93 (s, 10H), 0.16 (s, 7H).

### Example 1B

### (E)/(Z)-ethyl 2-acetoxy-3-(2-(benzyloxy)-5-((tert-butyldimethylsilyl)oxy)phenyl)acrylate

In a 50 mL Erlenmyer flask, ethyl 2-acetoxy-2-(diethoxyphosphoryl)acetate (37.1 g) was weighed and dried over anhydrous MgSO₄. The mixture was filtered over a 0.5 inch bed of silica and washed with toluene (50 mL) into a 1 L round bottom flask. The toluene mixture was concentrated and 200 mL tetrahydrofuran was added, followed by Cs₂CO₃ (42.8 g). The mixture was stirred at ambient temperature for 20 minutes. A tetrahydrofuran mixture (15 mL and 50 mL washing) of Example 1A (15 g) was added, and the reaction mixture was stirred at ambient temperature for 66 hours. The reaction mixture was filtered, the filtrate was transferred to a separatory funnel with 200 mL water, and the layers were separated. The aqueous layer was washed with ethyl acetate (2×100 mL), and the combined organic layers were washed with brine, dried over MgSO₄, filtered, and concentrated. The crude material was purified by silica gel chromatography over a 330 g column on a Grace Reveleris system (0-100% ethyl acetate/heptanes elution gradient). Fractions containing the desired product were combined, concentrated and dried under vacuum to obtain the title compound as an inseparable E/Z mixture. The E/Z ratio was found to be inconsequential for the subsequent step. ¹H NMR of Z isomer (tentatively assigned): ¹H NMR (400 MHz, DMSO-d₆) δ ppm 7.63 (s, 1H), 7.48-7.32 (m, 5H), 7.15 (d, 1H), 7.10 (d, 1H), 6.92 (dd, 1H), 5.13 (s, 2H), 4.20 (q, 2H), 2.27 (s, 3H), 1.23 (t, 3H), 0.94 (s, 9H), 0.16 (s, 6H). ¹H NMR of E isomer (tentatively assigned): ¹H NMR (400 MHz, DMSO-d₆) δ ppm 7.48-7.29 (m, 5H), 6.98 (d, 1H), 6.88 (s, 1H), 6.80 (d, 2H), 5.05 (s, 2H), 4.02 (q, 2H), 2.20 (s, 3H), 1.03 (t, 3H).

0.94 (s, 9H), 0.15 (s, 6H). MS (ESI) m/z 488.0 (M+NH₄)⁺.

### Example 1C

### (R)-ethyl 2-acetoxy-3-(2-(benzyloxy)-5-((tert-butyldimethylsilyl)oxy)phenyl)propanoate

A 100 mL Parr stainless steel reactor was charged with degassed methanol (37.5 mL) and Example 1B (10.5 g). In a nitrogen-filled glove box, a vial was charged with 1,2-Bis [(2R,5R)-2,5-diethylphospholano]benzene(1,5-cyclooctadiene)rhodium (I) trifluoromethanesulfonate (0.45 g) dissolved in degassed methanol (4 mL). The catalyst mixture was capped, brought outside the glove box, and added to the reactor via syringe. The reaction mixture was stirred under 50 psi of hydrogen at 35° C. for 8 hours. The reaction mixture was cooled to ambient temperature and filtered. The filtrate was concentrated. The crude material was purified on a silica plug with 20% ethyl acetate/heptanes as the eluent. The fractions containing the desired product were combined and concentrated to obtain the title compound. ¹H NMR (500 MHz, DMSO-d₆) δ ppm 7.48-7.43 (m, 2H), 7.41-7.36 (m, 2H), 7.35-7.29 (m, 1H), 6.93 (dt, 1H), 6.72-6.66 (m, 2H), 5.12 (dd, 1H), 5.09-5.00 (m, 2H), 4.03 (qd, 2H), 3.16 (dd, 1H), 2.96 (dd, 1H), 1.97 (s, 3H), 1.07 (t, 3H), 0.93 (s, 9H), 0.14 (s, 6H). MS (DCI) m/z 490.2 (M+NH₄)⁺. Enantiomeric excess was determined in the following way: A vial was charged with Example 1C (8 mg) and tetrahydrofuran (1 mL). A 1M mixture of TBAF (tetra-n-butylammonium fluoride) in tetrahydrofuran was added in a single portion. After 5 minutes, the reaction mixture was diluted with ethyl acetate (1 mL) and poured over water (1 mL). The biphasic mixture was vigorously stirred and the layers were allowed to separate. The organic layer was removed via a pipette, dried over MgSO₄, filtered, and concentrated. Analytical SFC: 5-50% methanol, ChiralPak IC column, retention time for the R enantiomer=2.28 minutes, retention time for the S enantiomer=2.08 minutes. The ee (enantiomeric excess) of the sample was determined to be >99%.

### Example 1D

### (R)-ethyl 2-acetoxy-3-(5-((tert-butyldimethylsilyl)oxy)-2-hydroxyphenyl)propanoate

Example 1C (10.2 g) in ethanol (70 mL) was added to 5% Pd/C (wet JM #9) (0.517 g) in a 250 mL pressure bottle. The mixture was stirred under 50 psi of hydrogen (g) at 35° C. for 7.5 hours. The reaction mixture was cooled to ambient temperature and was filtered. The filtrate was concentrated to obtain the title compound. ¹H NMR (400 MHz, DMSO-d₆) δ ppm 9.08 (s, 1H), 6.68-6.60 (m, 1H), 6.59-6.49 (m, 2H), 5.09 (dd, 1H), 4.05 (q, 2H), 3.02 (dd, 1H), 2.87 (dd, 1H), 1.99 (s, 3H), 1.11 (t, 3H), 0.92 (s, 9H), 0.11 (s, 6H). MS (ESI) m/z 399.8 (M+NH₄)⁺. Analytical SFC: 5-50% methanol, Whelk-01 (S,S) column, retention time for the R enantiomer=1.828 minutes, retention time for the S enantiomer=1.926 minutes. The ee (enantiomeric excess) of the sample was determined to be >99%.

### Example 1E

### 2-(2-(2-(2-(2-methoxyethoxy)ethoxy)ethoxy)phenyl)-4,4,5,5-tetramethyl-1,3,2-dioxaborolane

2-(4,4,5,5-Tetramethyl-1,3,2-dioxaborolan-2-yl)phenol (8.57 mL) and 2-(2-(2-methoxyethoxy)ethoxy)ethanol (7.58 mL) were added to tetrahydrofuran (200 mL). Triphenylphosphine (11.80 g) was added, and the mixture was stirred until it dissolved. (E)-Diisopropyldiazene-1,2-dicarboxylate (8.86 mL) was added, and the mixture was stirred at 50° C. for two days. The mixture was cooled, and the solvent was removed under reduced pressure. Diethyl ether (100 mL) and heptanes (50 mL) were added. The mixture was stirred vigorously to precipitate triphenylphosphine oxide. The mixture was filtered, concentrated and purified by flash column chromatography on silica gel using a 30-60% gradient of ethyl acetate in heptanes to provide the title compound. ¹H NMR (400 MHz, DMSO-d₆) δ ppm 7.48 (dd, 1H), 7.40 (td, 1H), 6.95-6.92 (m, 2H), 4.04 (t, 2H), 3.75 (t, 2H), 3.69 (t, 2H), 3.54-3.48 (m, 4H), 3.43-3.41 (m, 2H), 3.23 (s, 3H), 1.22-1.12 (m, 12H).

### Example 1F

### (2-(2-(2-(2-(2-methoxyethoxy)ethoxy)ethoxy)phenyl)pyrimidin-4-yl)methanol

Example 1E (7.80 g) and (2-bromopyrimidin-4-yl)methanol (4.43 g) were dissolved in 1,4-dioxane (90 mL). Aqueous sodium carbonate (2 M, 31.9 mL) was added. The mixture was degassed and flushed with nitrogen three times. Dichloro[1,1′-bis(diphenylphosphino)ferrocene]palladium (II) dichloromethane adduct (1.739 g) was added, and the mixture was degassed and flushed with nitrogen once. The mixture was stirred at 75° C. for 16 hours. The mixture was cooled, diluted with ethyl acetate (100 mL), washed with water (50 mL), washed with brine (50 mL), and dried on anhydrous sodium sulfate. The mixture was filtered, concentrated and purified by flash column chromatography on silica gel using a 0-7% gradient of methanol in dichloromethane to provide the title compound. ¹H NMR (400 MHz, DMSO-d₆) δ ppm 8.84 (d, 1H), 7.53 (dd, 1H), 7.48 (d, 1H), 7.42 (dt, 1H), 7.15 (d, 1H), 7.05 (t, 1H), 5.64 (t, 1H), 4.59 (d, 2H), 4.11 (t, 2H), 3.66 (t, 2H), 3.50-3.48 (m, 2H), 3.46-3.43 (m, 4H), 3.40-3.38 (m, 2H), 3.22 (s, 3H). MS (ESI) m/z 349.3 (M+H)⁺.

### Example 1G

### ethyl (R)-2-acetoxy-3-(5-((tert-butyldimethylsilyl)oxy)-2-((2-(2-(2-(2-(2-methoxyethoxy)ethoxy)ethoxy)phenyl)pyrimidin-4-yl)methoxy)phenyl)propanoate

Triphenylphosphine (575 mg) and (E)-N¹,N¹,N²,N²-tetramethyldiazene-1,2-dicarboxamide (377 mg) were mixed in tetrahydrofuran (4.5 mL) at 0° C. for 20 minutes. The mixture was added to Example 1F (496 mg) and Example 1D (419 mg) which had been added to tetrahydrofuran (1 mL) in a separate flask and pre-cooled to 0° C. The mixture was stirred at 0° C. for one hour and at room temperature for 16 hours. The mixture was filtered, washing with ethyl acetate (10 mL). The mixture was concentrated under vacuum and was purified by flash column chromatography on silica gel using a gradient of 70-100% ethyl acetate in heptanes to provide the title compound. ¹H NMR (400 MHz, DMSO-d₆) δ ppm 8.92 (d, 1H), 7.53 (dd, 1H), 7.48 (d, 1H), 7.44 (td, 1H), 7.16 (d, 1H), 7.06 (t, 1H), 6.94 (d, 1H), 6.76 (d, 1H), 6.71 (dd, 1H), 5.22-5.14 (m, 3H), 4.12 (t, 2H), 4.08 (qd, 2H), 3.67 (t, 2H), 3.50-3.48 (m, 2H), 3.41 (m, 4H), 3.35-3.33 (m, 2H), 3.27 (dd, 1H), 3.17 (s, 3H), 3.05 (dd, 1H), 1.99 (s, 3H), 1.11 (t, 3H), 0.92 (s, 9H), 0.15 (s, 6H). MS (APCI) m/z 713.7 (M+H)⁺.

### Example 1H

### ethyl (R)-3-(5-((tert-butyldimethylsilyl)oxy)-2-((2-(2-(2-(2-(2-methoxyethoxy)ethoxy)ethoxy)phenyl)pyrimidin-4-yl)methoxy)phenyl)-2-hydroxypropanoate

Example 1G (1218 mg) was dissolved in ethanol (9 mL). Sodium ethoxide (21.5% in ethanol, 28 mg, 0.032 mL) was added, and the mixture was stirred at room temperature for 2.5 hours. Acetic acid (0.015 mL) was added, and the mixture was stirred at room temperature for 10 minutes. The mixture was concentrated under vacuum and was purified by flash column chromatography on silica gel using a gradient of 70-100% ethyl acetate in heptanes to provide the title compound. ¹H NMR (400 MHz, DMSO-d₆) δ ppm 8.91 (d, 1H), 7.53 (dd, 1H), 7.48 (d, 1H), 7.44 (td, 1H), 7.16 (d, 1H), 7.06 (t, 1H), 6.89 (d, 1H), 6.73 (d, 1H), 6.66 (dd, 1H), 5.52 (d, 1H), 5.16 (m, 2H), 4.31 (q, 1H), 4.12 (t, 2H), 4.05 (qd, 2H), 3.67 (t, 2H), 3.51-3.48 (m, 2H), 3.41 (m, 4H), 3.36-3.24 (m, 2H), 3.18 (s, 3H), 3.10 (dd, 1H), 2.81 (dd, 1H), 1.12 (t, 3H), 0.93 (s, 9H), 0.14 (s, 6H). MS (ESI) m/z 671.5 (M+H)⁺.

### Example 1I

### 6-iodothieno[2,3-d]pyrimidin-4(3H)-one

Acetic acid (312 mL), sulfuric acid (9.37 mL) and water (63 mL) were combined with stirring. Thieno[2,3-d]pyrimidin-4(3H)-one (50 g), periodic acid (37.4 g) and iodine (75 g) were added sequentially, and the mixture was slightly endothermic. A heating mantle was added and the reaction mixture was ramped up to 60° C. Midway through, the temperature climbed to 68-69° C. The heating mantle was removed and the temperature was maintained at 70° C. by self-heating for about 45 minutes. LC/MS indicated a single peak corresponding to desired product. The reaction mixture was cooled to room temperature. The resulting suspension was filtered, washed with 5:1 acetic acid:water (three times) and diethyl ether (five times) to provide the title compound which was used in the next step without further purification. ¹H NMR (400 MHz, DMSO-d₆) δ ppm 12.80-12.41 (m, 1H), 8.10 (s, 1H), 7.66 (s, 1H). MS (ESI) m/z 277.9 (M−H)⁻.

### Example 1J

### 4-chloro-6-iodothieno[2,3-d]pyrimidine

Phosphorous oxychloride (37 mL) and N,N-dimethylaniline (11.5 mL) were combined, and Example 1I (25 g) was added over a few minutes. The reaction mixture was stirred at about 105° C. for 1.5 hours. An aliquot was analyzed by LC/MS, which indicated the reaction was complete. The suspension was cooled to 5-10° C. filtered, and washed with heptanes. The crude filter cake was dumped into ice water (uneventful) with rapid stirring. The mixture was stirred for about 30 minutes, filtered, washed with additional water (three times), washed with diethyl ether (three times) and dried on the filter bed overnight to provide the title compound which was used in the next step without further purification. ¹H NMR (400 MHz, DMSO-d₆) δ ppm 8.89 (s, 1H), 7.95 (s, 1H).

### Example 1K

### 5-bromo-4-chloro-6-iodothieno[2,3-d]pyrimidine

Example 1J (20.5 g) was taken up in acetonitrile (173 mL) and NBS (N-bromosuccinimide, 13.54 g) was added followed by tetrafluoroboric acid-dimethyl ether complex (2 mL). While the reaction was stirring, the temperature slowly climbed, reaching 25.5° C. after 30 minutes. The reaction mixture was allowed to stir overnight at room temperature. An additional 0.4 equivalents of NBS (N-bromosuccinimide) were added followed by tetrafluoroboric acid-dimethyl ether complex (2 mL), and the reaction mixture was stirred for an additional 5 hours. The reaction mixture was cooled in an ice bath to about 5° C. (internal) and filtered. The solids were washed with acetonitrile (twice) and dried on the filter bed overnight. The title compound was used in the next step without further purification. ¹H NMR (400 MHz, DMSO-d₆) δ ppm 8.93 (s, 1H).

### Example 1L

### 5-bromo-4-chloro-6-(4-fluorophenyl)thieno[2,3-d]pyrimidine

(Tris(dibenzylideneacetone) dipalladium (0)) (7.32 g), di-tert-butyl (2′,4′,6′-triisopropyl-[1,1′-biphenyl]-2-yl)phosphine (7.47 g), tripotassium phosphate (181 g). (4-fluorophenyl)boronic acid (89 g), and Example 1K (200 g) were combined in a three neck, 5 L round bottom flask, fit with water condenser, thermocouple/JKEM, overhead stirring and argon gas inlet. The material was inerted with argon for 40 minutes. Tetrahydrofuran (1705 mL) and water (426 mL) were combined into a 3 L round bottom flask and the subsurface was sparged for 30 minutes. The solvent mixture was then cannulated into the flask containing the material, observing a sharp temperature increase to 37° C. The temperature was set to 64° C. (internal), and the reaction mixture was stirred overnight (16 hours) under a light positive flow of argon. The reaction mixture was cooled to 38° C. and 200 mL water was added with stirring (overhead). Stirring was continued for 2 hours, and the material was filtered, washing with water. A second crop was obtained from the filtrate and was combined with the first crop. The combined material was taken up in hot tetrahydrofuran (2 L), stirred with 20 g thiosilica gel and charcoal for 30 minutes and filtered through a pad of diatomaceous earth. The filtrate was concentrated to provide the title compound. ¹H NMR (400 MHz, Chloroform-d) δ ppm 8.86 (s, 1H), 7.75-7.58 (m, 2H), 7.22 (t, 2H). MS (ESI) m/z 344.8 (M+H)⁺.

### Example 1M

### ethyl (R)-2-((5-bromo-6-(4-fluorophenyl)thieno[2,3-d]pyrimidin-4-yl)oxy)-3-(5-((tert-butyldimethylsilyl)oxy)-2-((2-(2-(2-(2-(2-methoxyethoxy)ethoxy)ethoxy)phenyl)pyrimidin-4-yl)methoxy)phenyl)propanoate

Example 1H (878 mg), Example 1L (472 mg) and cesium carbonate (1279 mg) were heated in tert-butyl alcohol (5.5 mL) at 65° C. for three hours. The mixture was cooled and was diluted with a mixture of ethyl acetate and methyl tert-butyl ether (1:1, 15 mL). The mixture was vacuum filtered over a pad of diatomaceous earth, washing with a mixture of ethyl acetate and methyl tert-butyl ether (1:1, 10 mL). The filtrate was washed with water (8 mL), and a small amount of brine (1 mL) was used to break up the emulsion. The aqueous layer was washed with brine (5 mL), dried on anhydrous sodium sulfate, and filtered. The filtrate was concentrated under vacuum and was purified by flash column chromatography on silica gel using a gradient of 70-100% ethyl acetate in heptanes to provide the title compound, ¹H NMR (400 MHz, DMSO-d₆) δ ppm 8.88 (d, 1H), 8.62 (s, 1H), 7.71 (m, 2H), 7.53 (dd, 1H), 7.48 (d, 1H), 7.45-7.38 (m, 3H), 7.16 (d, 1H), 7.04 (t, 1H), 6.96-6.92 (m, 2H), 6.68 (dd, 1H), 5.85 (dd, 1H), 5.19 (m, 2H), 4.16 (q, 2H), 4.11 (t, 2H), 3.66 (t, 2H), 3.57 (dd, 1H), 3.49-3.46 (m, 2H), 3.40 (m, 4H), 3.33-3.25 (m, 3H), 3.15 (s, 3H), 1.14 (t, 3H), 0.85 (s, 9H), 0.06 (s, 3H), 0.04 (s, 3H). MS (ESI) m/z 977.4, 979.3 (M+H)⁺.

### Example 1N

### (S)-2,3-dihydroxypropyl 4-methylbenzenesulfonate

To a stirring mixture of (S)-(2,2-dimethyl-1,3-dioxolan-4-yl)methyl 4-methylbenzenesulfonate (9 g) in 36 mL of methanol was slowly added 42 mL of 1 M aqueous HCl mixture, and the reaction was stirred at ambient temperature overnight. The mixture was concentrated under reduced pressure to remove most of the methanol. The mixture was carefully poured into 225 mL of saturated aqueous sodium bicarbonate mixture. The mixture was extracted with three portions of ethyl acetate. The combined organic layers were washed with saturated aqueous brine, dried over anhydrous magnesium sulfate, filtered and concentrated onto silica gel. Purification by silica gel flash chromatography on a CombiFlash® Teledyne Isco system using a Teledyne Isco RediSep® Rf gold 330 g silica gel column (eluting with 10-80% of 2:1 ethyl acetate:ethanol in heptane) provided the title compound, which was quickly used in the next step before it solidified. ¹H NMR (400 MHz, DMSO-d₆) δ ppm 2.42 (s, 3H), 3.18-3.27 (m, 1H), 3.29-3.34 (m, 1H), 3.61 (ttd, 1H), 3.84 (dd, 1H), 3.97-4.05 (m, 1H), 4.68 (t, 1H), 5.10 (d, 1H), 7.48 (d, 2H), 7.73-7.85 (m, 2H). LC/MS (APCI) m/z 247.3 (M+H)⁺.

### Example 10

### (S)-3-(bis(4-methoxyphenyl)(phenyl)methoxy)-2-hydroxypropyl 4-methylbenzenesulfonate

To a stirring mixture of Example 1N (6.3 g) in 128 mL of dichloromethane at 0° C., was added 4,4′-dimethoxytrityl chloride (9.10 g) in one portion. To the mixture was added N,N-diisopropylethylamine (4.69 mL) dropwise over 15 minutes. The reaction mixture was stirred at 0° C. for an hour and was quenched with saturated aqueous ammonium chloride (100 mL). The layers were separated, and the aqueous layer was extracted with two portions of dichloromethane. The combined organic extracts were dried over anhydrous magnesium sulfate, filtered and concentrated onto silica gel. Purification by flash chromatography on a CombiFlash® Teledyne Isco system using a Teledyne Isco RediSep® Rf gold 330 g silica gel column (eluting 0-50% ethyl acetate/heptane) provided the title compound, ¹H NMR (400 MHz, DMSO-d₆) δ ppm 2.39 (s, 3H), 2.84 (dd, 1H), 2.94 (dd, 1H), 3.74 (s, 6H), 3.76-3.81 (m, 1H), 3.96 (dd, 1H), 4.02-4.09 (m, 1H), 5.28 (d, 1H), 6.82-6.92 (m, 4H), 7.12-7.18 (m, 4H), 7.19-7.25 (m, 1H), 7.28 (d, 4H), 7.45 (d, 2H), 7.71-7.79 (m, 2H).

### Example 1P

### (4-bromo-2-chlorophenoxy)triisopropylsilane

To a mixture of 4-bromo-2-chlorophenol (570 g) in dichloromethane (4.5 L) was added triisopropylchlorosilane (582 mL) and imidazole (187 g), and the mixture was stirred for 8 hours at 25° C. The reaction mixture was poured into water, and was extracted with dichloromethane (3×2000 mL). The organic layers were combined, washed with brine (1×2000 mL), dried over anhydrous sodium sulfate, filtered and concentrated under reduced pressure to give a residue. The residue was purified by column chromatography on silica gel, eluting with petroleum ether to obtain the title compound. ¹H NMR (400 MHz, chloroform-d) δ ppm 1.12 (d, 18H), 1.27-1.35 (m, 3H), 6.78 (d, 1H), 7.21 (dd, 1H), 7.49 (d, 1H).

### Example 1Q

### (4-bromo-2-chloro-3-methylphenoxy)triisopropylsilane

A 5 L, 3-neck round-bottom flask, fitted with overhead stirring, nitrogen inlet and outlet, three addition funnels, a thermocouple and a Claisen adaptor was twice dried with a torch and heat gun and cooled under nitrogen. The reaction flask was charged with N,N-diisopropylamine (69.2 mL) and tetrahydrofuran (2110 mL). The mixture was cooled to −78° C. under nitrogen, n-Butyllithium (177 mL, 2.5 M in hexane) was added slowly via addition funnel, and a slight rise in temperature was observed.

The mixture was stirred at −78° C. for 45 minutes, at which time Example 1P (153.5 g) was added over 30 minutes as a tetrahydrofuran (200 mL) mixture. The reaction mixture was stirred for about 6.5 hours at −76° C. Iodomethane (31.7 mL) was added dropwise via addition funnel, maintaining the temperature below −62° C. The reaction mixture was allowed to warm slowly overnight to room temperature. The volatiles were removed by rotary evaporation. Ethyl acetate (1.5 L) and water (1.5 L) were added to the residue, and the layers were separated. The organics were washed with brine. The combined aqueous layer was extracted once with ethyl acetate (500 mL). The combined organics were dried (MgSO₄), filtered and concentrated by rotary evaporation. The residue was purified by flash silica gel column in chromatography (1500 g SiO₂, heptanes) to provide the title compound.

### Example 1R

### 4-bromo-2-chloro-3-methylphenol

To a mixture of Example 1Q (500 g) in tetrahydrofuran (5 L) was added tetra-N-butylammonium fluoride (381 g). The reaction mixture was stirred at 25° C. for 3 hours. The reaction mixture was diluted with water (3 L), and extracted with tert-butyl methyl ether (3×2 L). The combined organic layers were dried over anhydrous sodium sulfate, filtered and concentrated under reduced pressure. The residue was diluted with 10% (w/w) aqueous sodium hydroxide (8 L) and washed with a mixture of petroleum ether/tert-butyl methyl ether (v/v=10/1, 3×3 L). The organic layer was discarded. The aqueous layer was adjusted to pH=3 with 3 N aqueous HCl mixture and was extracted with a mixture of petroleum ether/tert-butyl methyl ether (v/v=10/1, 3×4 L). The combined organic layers were dried over anhydrous sodium sulfate, filtered and concentrated under reduced pressure to give a residue. The residue was triturated with petroleum ether (1.5 L), and the material was dried under high vacuum to provide the title compound. ¹H NMR (400 MHz, chloroform-d) δ ppm 2.51 (s, 3H) 5.60 (s, 1H) 6.80 (d, 1H) 7.37 (d, 1H).

### Example 1S

### (R)-3-(bis(4-methoxyphenyl) (phenyl)methoxy)-2-(4-bromo-2-chloro-3-methylphenoxy) propyl 4-methylbenzenesulfonate

A 500 mL round bottom flask, equipped with stir bar and a thermometer, was loaded with Example 10 (10.2 g). Example 1R (4.94 g) and triphenylphosphine (7.31 g). Tetrahydrofuran (186 mL) was added, and to the resulting stirring mixture di-tert-butyl azodicarboxylate (6.42 g) was added portionwise, while keeping the temperature below 25° C. After the addition, the flask was capped, evacuated, and backfilled twice with nitrogen. The reaction mixture was placed in a 45° C. pre-heated oil bath, and the mixture was stirred for 90 minutes. After cooling to ambient temperature, the mixture was concentrated onto silica gel. Purification by flash chromatography on a CombiFlash® Teledyne Isco system using a Teledyne Isco RediSep® Rf gold 330 g silica gel column (eluting 5-40% ethyl acetate/heptane) provided a mixture of the desired product and hydrazine by-product. An additional purification by flash chromatography was performed using the same instrument and column but with a 10-100% dichloromethane/heptane gradient to obtain the title compound. Analytical SFC was performed on an Aurora A5 SFC Fusion and Agilent 1100 system running under Agilent Chemstation software control. The SFC system included a 10-way column switcher, CO₂ pump, modifier pump, oven, and backpressure regulator. The mobile phase comprised of supercritical CO₂ supplied by a beverage-grade CO₂ cylinder with a modifier mixture of methanol at a flow rate of 3 mL/minute. Oven temperature was at 35° C. and the outlet pressure was at 150 bar. The mobile phase gradient started with 5% modifier and was held for 0.1 minutes at a flow rate of 1 mL/minute, and the flow rate was ramped up to 3 mL/minute and was held for 0.4 minutes. The modifier was ramped from 5% to 50% over the next 8 minutes at 3 mL/minute and was held for 1 minute at 50% modifier (3 mL/minute). The gradient was ramped down from 50% to 5% modifier over 0.5 minute (3 mL/minute). The instrument was fitted with a Whelk-01 (S,S) column with dimensions of 4.6 mm i.d.×150 mm length with 5 μm particles. Minor enantiomer (R) eluted after 7.3 minutes and major enantiomer (S) eluted after 7.8 minutes. Using this assay the enantiopurity of title compound was determined to be 96% ee (enantiomeric excess). ¹H NMR (400 MHz, DMSO-d₆) δ ppm 2.33 (s, 3H), 2.41 (s, 3H), 3.16 (d, 2H), 3.69 (d, 6H), 4.19-4.31 (m, 2H), 4.75 (p, 1H), 6.74-6.86 (m, 5H), 7.06-7.12 (m, 4H), 7.13-7.20 (m, 1H), 7.20-7.25 (m, 4H), 7.31-7.37 (m, 2H), 7.39 (d, 1H), 7.61-7.70 (m, 2H).

### Example 1T

### (R)-3-(bis(4-methoxyphenyl)(phenyl)methoxy)-2-(2-chloro-3-methyl-4-(4,4,5,5-tetramethyl-1,3,2-dioxaborolan-2-yl)phenoxy) propyl 4-methylbenzenesulfonate

An 8 mL microwave vial, equipped with stir bar, was charged with potassium acetate (2.036 g), bis(pinacolato)diboron (3.16 g) and [1,1′-bis(diphenylphosphino)ferrocene]dichloropalladium dichloride (0.379 g). A mixture of Example 1S (7.8 g) in 2-methyltetrahydrofuran (51.9 mL) was added. The flask was capped with a septa, and nitrogen was bubbled through the mixture for 15 minutes. The mixture was stirred at 90° C. for 5 hours. The mixture was cooled and filtered through a diatomaceous earth pad and the filter cake was washed with ethyl acetate (˜75 mL). The mixture was concentrated onto silica gel, and purification by flash chromatography (Isco, 330 G Gold Redi-Sep column, 5-40% ethyl acetate/heptane) provided the title compound. ¹H NMR (400 MHz, DMSO-d₆) δ ppm 1.30 (s, 12H), 2.35 (s, 3H), 2.53 (s, 3H), 3.20 (d, 2H), 3.72 (d, 6H), 4.22-4.38 (m, 2H), 4.77-4.90 (m, 1H), 6.74-6.87 (m, 5H), 7.10-7.17 (m, 4H), 7.17-7.30 (m, 5H), 7.32-7.38 (m, 2H), 7.43 (d, 1H), 7.65-7.71 (m, 2H).

### Example 1U

### ethyl (R)-2-((5-((1S)-4-(((R)-1-(bis(4-methoxyphenyl) (phenyl)methoxy)-3-(tosyloxy) propan-2-yl)oxy)-3-chloro-2-methylphenyl)-6-(4-fluorophenyl)thieno[2,3-d]pyrimidin-4-yl)oxy)-3-(5-((tert-butyldimethylsilyl)oxy)-2-((2-(2-(2-(2-(2-methoxyethoxy)ethoxy)ethoxy)phenyl)pyrimidin-4-yl)methoxy)phenyl)propanoate

Example 1M (898 mg), Example 1T (954 mg), cesium carbonate (897 mg), and bis(di-tert-butyl (4-dimethylaminophenyl)-phosphine)dichloropalladium (II) (65 mg) were added to a flask. A mixture of tetrahydrofuran (9 mL) and water (2.25 mL) that had been degassed and flushed with nitrogen three times was added to the solids. The mixture was stirred at room temperature for 16 hours. The mixture was diluted with ethyl acetate (10 mL) and water (2 mL). The layers were separated, and the aqueous layer was extracted with ethyl acetate (10 mL) twice. The organic extracts were combined, washed with brine (10 mL), dried on anhydrous sodium sulfate, and filtered. The filtrate was concentrated by rotary evaporation with an ambient water bath and was purified by flash column chromatography on silica gel using a gradient of 70-100% ethyl acetate in heptanes. The solvent was removed by rotary evaporation with an ambient water bath to provide the title compound. MS (ESI) m/z 1596.2 (M+H)⁺.

### Example 1V

### ethyl (R)-2-((5-((1S)-4-(((R)-1-(bis(4-methoxyphenyl)(phenyl)methoxy)-3-(tosyloxy) propan-2-yl)oxy)-3-chloro-2-methylphenyl)-6-(4-fluorophenyl)thieno[2,3-d]pyrimidin-4-yl)oxy)-3-(5-hydroxy-2-((2-(2-(2-(2-(2-methoxyethoxy)ethoxy)ethoxy)phenyl)pyrimidin-4-yl)methoxy)phenyl)propanoate

Example 1U (915 mg) was dissolved in dichloromethane (30 mL). Tetra-N-butylammonium fluoride (1 M in tetrahydrofuran, 0.58 mL) was added and the mixture was stirred at room temperature for 15 minutes. The mixture was concentrated by rotary evaporation with an ambient water bath and was purified by flash column chromatography on silica gel using a gradient of 70-100% ethyl acetate in heptanes. The solvent was removed by rotary evaporation with an ambient water bath to provide the title compound. MS (ESI) m/z 1456.2 (M+H)⁺.

### Example 1W

### ethyl (7R,16S,21S)-16-{[bis(4-methoxyphenyl) (phenyl)methoxy]methyl}-19-chloro-1-(4-fluorophenyl)-10-{[2-(2-{2-[2-(2-methoxyethoxy)ethoxy]ethoxy}phenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 1V (684 mg) was dissolved in N,N-dimethylformamide (47 mL). Cesium carbonate (1531 mg) was added, and the mixture was stirred at room temperature for 5.5 hours. The mixture was diluted with water (150 mL) and ethyl acetate (100 mL). The layers were separated, and the aqueous layer was extracted with ethyl acetate (100 mL) two times. The organic extracts were combined and washed with water (50 mL) and brine (50 mL). The mixture was dried over anhydrous sodium sulfate, filtered, concentrated by rotary evaporation with an ambient water bath, and purified by flash column chromatography on silica gel using a gradient of 70-100% ethyl acetate in heptanes. The solvent was removed by rotary evaporation with an ambient water bath to provide the title compound. MS (ESI) m/1283.4 (M+H)⁺.

### Example 1X

### ethyl (7R,16R,21S)-19-chloro-1-(4-fluorophenyl)-16-(hydroxymethyl)-10-{[2-(2-{2-[2-(2-methoxyethoxy)ethoxy]ethoxy}phenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 1W (525 mg) was dissolved in dichloromethane (2 mL) and methanol (2 mL). Formic acid (2 mL) was added, and the mixture was stirred at room temperature for 15 minutes. The mixture was poured slowly into a saturated aqueous sodium bicarbonate mixture (20 mL) and was extracted with ethyl acetate (50 mL). The organic layer was washed with brine (10 mL), dried over anhydrous sodium sulfate, and filtered. The filtrate was concentrated and purified by flash column chromatography on silica gel using a gradient of 70-100% ethyl acetate in heptanes. The solvent was removed by rotary evaporation with an ambient water bath to provide the title compound. ¹H NMR (400 MHz, DMSO-d₆) δ 8.94 ppm (d, 1H), 8.72 (s, 1H), 7.62 (m, 1H), 7.61-7.55 (m, 2H), 7.44 (m, 2H), 7.24-7.14 (m, 4H), 7.08 (t, 1H), 6.98 (d, 1H), 6.93 (d, 1H), 6.85 (dd, 1H), 6.08 (m, 1H), 5.56 (d, 1H), 5.18-5.09 (m, 3H), 4.99 (t, 1H), 4.46-4.42 (m, 1H), 4.40-4.36 (m, 2H), 4.15-4.10 (m, 3H), 3.94-3.78 (m, 3H), 3.68 (m, 4H), 3.58 (m, 1H), 3.51-3.47 (m, 3H), 3.43 (m, 2H), 3.41-3.35 (m, 2H), 3.17-3.14 (m, 1H), 2.87 (dd, 1H), 2.25 (s, 3H), 0.80 (t, 3H). MS (ESI) m/z 981.5 (M+H)⁺.

### Example 1Y

### ethyl (7R,16S,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

Example 1X (282 mg) was dissolved in dichloromethane (3 mL). Triethylamine (87 mg, 0.12 mL) was added followed by 4-methylbenzene-1-sulfonyl chloride (110 mg). The mixture was stirred at room temperature for 16 hours. The mixture was concentrated and was purified by flash column chromatography on silica gel using a gradient of 70-100% ethyl acetate in heptanes. The solvent was removed by rotary evaporation with an ambient water bath to provide the title compound. ¹H NMR (400 MHz, DMSO-d₆) δ ppm 8.94 (d, 1H), 8.72 (s, 1H), 7.81 (d, 2H), 7.63 (m, 1H), 7.58 (dd, 1H), 7.56 (d, 1H), 7.46 (d, 2H), 7.23-7.16 (m, 5H), 7.09 (d, 2H), 6.97 (d, 1H), 6.93 (d, 1H), 6.89-6.86 (m, 1H), 6.09 (m, 1H), 5.51 (d, 1H), 5.16 (m, 3H), 4.61 (m, 1H), 4.39-4.27 (m, 4H), 4.15-4.10 (m, 2H), 3.94-3.76 (m, 2H), 3.69-3.64 (m, 2H), 3.52-3.48 (2H), 3.43 (m, 2H), 3.39-3.35 (m, 2H), 3.19 (s, 3H), 3.16-3.14 (m, 1H), 2.86 (dd, 1H), 2.44 (d, 1H), 2.39 (s, 3H), 2.22 (s, 3H), 0.79 (t, 3H). MS (ESI) m/z 1135.5 (M+H)⁺.

### Example 1Z

### ethyl (7R,16R,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 1Y (271 mg) and 1-methylpiperazine (717 mg) were dissolved in N,N-dimethylformamide (1 mL) and the reaction mixture was heated to 40° C. for 18.5 hours. Water (15 mL) was added while stirring the mixture vigorously. The precipitate was vacuum filtered, washed with water (10 mL), and dried under vacuum. The isolated material was used in the next step without further purification. ¹H NMR (400 MHz, DMSO-d₆) δ ppm 8.92 (d, 1H), 8.73 (s, 1H), 7.65 (m, 1H), 7.59 (dd, 1H), 7.48-7.42 (m, 2H), 7.25-7.14 (m, 5H), 7.08 (t, 1H), 6.97 (d, 1H), 6.90 (d, 1H), 6.82 (dd, 1H), 6.15 (m, 1H), 5.57 (d, 1H), 5.12 (m, 3H), 4.52-4.30 (m, 4H), 4.15-4.11 (m, 3H), 3.89 (m, 2H), 3.84-3.78 (m, 1H), 3.69 (m, 2H), 3.52-3.47 (m, 2H), 3.43 (m, 2H), 3.39-3.35 (m, 2H), 3.19 (s, 3H), 2.89 (d, 1H), 2.72 (d, 1H), 2.58-2.54 (m, 2H), 2.40-2.29 (m, 6H), 2.25 (s, 3H), 2.11 (s, 3H), 0.79 (t, 3H). MS (ESI) m/z 1063.5 (M+H)⁺.

### Example 1AA

### (7R,16R,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 1Z (211 mg) was dissolved in tetrahydrofuran (2 mL) and methanol (1 mL). Lithium hydroxide monohydrate (166 mg) in water (1.5 mL) was added. The mixture was stirred at room temperature for 16 hours. The reaction mixture was quenched with acetic acid (0.27 mL) and was stirred for five minutes at room temperature. The mixture was concentrated under vacuum and was diluted with dimethyl sulfoxide (1 mL) and acetonitrile (1 mL). The crude material was purified by reverse phase using a 30-80% gradient of acetonitrile in water (with 0.1% trifluoroacetic acid) over 40 minutes on a Grace Reveleris equipped with a Luna™ column: C18(2), 100 Å, 250×50 mm. The fractions containing the desired compound were combined, frozen and lyophilized to isolate the title compound as the bistrifluoroacetic acid salt. ¹H NMR (400 MHz, DMSO-d₆) δ ppm 8.89 (d, 1H), 8.75 (s, 1H), 7.59 (dd, 1H), 7.53 (d, 1H), 7.46 (td, 1H), 7.22-7.18 (m, 5H), 7.15 (d, 1H), 7.08 (t, 1H), 6.97 (d, 1H), 6.89 (d, 1H), 6.83 (dd, 1H), 6.17 (m, 1H), 5.68 (d, 1H), 5.18 (q, 2H), 4.59 (m, 1H), 4.47 (d, 1H), 4.37 (m, 1H), 4.14 (t, 2H), 3.88 (dd, 1H), 3.69 (t, 2H), 3.53-3.50 (m, 2H), 3.44 (m, 4H), 3.39-3.35 (m, 4H), 3.19 (s, 3H), 3.17-3.08 (m, 5H), 2.91 (d, 2H), 2.78 (s, 3H), 2.73 (t, 2H), 2.22 (s, 3H). MS (ESI) m/z 1035.2 (M+H)⁺.

### Example 2

### (7S,16R,21S)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was isolated during the synthesis of Example 1AA as the bistrifluoroacetic acid salt. ¹H NMR (400 MHz, DMSO-d₆) δ ppm 8.90 (d, 1H), 8.70 (s, 1H), 7.66 (d, 1H), 7.58 (dd, 1H), 7.47 (td, 1H), 7.37-7.18 (m, 6H), 7.09 (t, 1H), 6.98 (d, 1H), 6.94 (d, 1H), 6.80 (dd, 1H), 6.74 (d, 1H), 5.90 (d, 1H), 5.79 (dd, 1H), 5.22 (q, 2H), 4.88 (m, 1H), 4.28 (dd, 1H), 4.21-4.13 (m, 3H), 3.82 (dd, 1H), 3.71 (m, 2H), 3.52 (m, 2H), 3.48-3.42 (m, 6H), 3.37 (m, 2H), 3.29-3.04 (m, 4H), 3.20 (s, 3H), 3.01-2.83 (m, 4H), 2.83 (s, 3H), 2.51 (s, 3H). MS (ESI) m/z 1035.3 (M+H)⁺.

### Example 3

### (7R,16R,21R)-19-chloro-1-(4-fluorophenyl)-10-{[2-(2-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was isolated during the synthesis of Example 1AA as the bistrifluoroacetic acid salt. ¹H NMR (400 MHz, DMSO-d₆) δ ppm 8.89 ppm (d, 1H), 8.65 (s, 1H), 7.70 (d, 1H), 7.59 (dd, 1H), 7.48 (td, 1H), 7.34 (m, 2H), 7.24 (t, 2H), 7.20 (d, 1H), 7.09 (m, 2H), 6.87 (d, 1H), 6.79 (dd, 1H), 6.66 (d, 1H), 6.08 (d, 1H), 5.80 (dd, 1H), 5.21 (q, 2H), 5.17 (m, 1H), 4.43 (d, 2H), 4.15 (t, 2H), 4.11 (m. 2H), 3.70 (t, 2H), 3.54 (m, 2H), 3.42 (m, 6H), 3.35 (m, 2H), 3.19 (s, 3H), 3.16-3.06 (m, 4H), 2.93 (m, 2H), 2.83 (s, 3H), 2.66-2.58 (m, 2H), 2.50 (s, 3H).

### Example 4

### (7R,16R,21S)-19-chloro-1-(4-fluorophenyl)-10-{[2-(2-methoxyphenyl)pyrimidin-4-yl]methoxy}-20-methyl-16-{[4-(2,5,8,11-tetraoxatridecan-13-yl) piperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 4A

### 2,5,8,11-tetraoxatridecan-13-yl 4-methylbenzenesulfonate

3,6,9,12-Tetraoxatetradecan-1-ol (3 g) was dissolved in anhydrous CH₂Cl₂ (16 mL) and triethylamine (4.82 mL). To the mixture was added p-toluenesulfonyl chloride (3.30 g). The mixture was stirred at ambient temperature overnight, diluted with CH₂Cl₂, and washed with water. The organics were dried over MgSO₄, filtered, and concentrated. The residue was purified by silica gel flash chromatography on an AnaLogix IntelliFlash²⁸⁰ system (20-100% ethyl acetate/hexanes, linear gradient) to provide the title compound. LC/MS (APCI) m/z 363.3 (M+H)⁺.

### Example 4B

### tert-butyl 4-(2,5,8,11-tetraoxatridecan-13-yl)piperazine-1-carboxylate

Example 4A (1.8 g) was dissolved in anhydrous acetonitrile (16 mL) and triethylamine (1.384 mL). To the mixture was added tert-butyl piperazine-1-carboxylate (1.110 g) and the mixture was heated under reflux overnight. The mixture was concentrated and was purified by silica gel flash chromatography on an AnaLogix IntelliFlash²⁸⁰ system (eluting with 20% methanol/CH₂Cl₂) to provide the title compound. LC/MS (ESI) m/z 377.2 (M+H)⁺.

### Example 4C

### 1-(2,5,8,11-tetraoxatridecan-13-yl)piperazine

To a mixture of Example 4B (1.60 g) in anhydrous CH₂Cl₂ (5 mL) was added trifluoroacetic acid (4.91 mL). The mixture was stirred at ambient temperature for one hour, and was concentrated in vacuo. The residue was dissolved in 2 mL of 50% methanol in CH₂Cl₂ and was loaded on a 10G MEGA BE-SCX Bond Elut resin cartridge. The cartridge was eluted with 2M ammonia in methanol. The filtrate was collected and was concentrated to provide the title compound. MS (ESI) m/z 277.3 (M+H)⁺.

### Example 4D

### 2-methoxybenzimidamide hydrochloride

An oven-dried 12 L five-necked flask equipped with a mechanical stirrer, a gas inlet with tubing leading to a nitrogen regulator, a gas inlet adapter with tubing leading to a bubbler, and an internal temperature probe (J-KEM controlled) was charged with ammonium chloride (86 g). The material was mixed under nitrogen with anhydrous toluene (2 L). The mixture was cooled to −12.3° C. in an ice/methanol bath. To the mixture was added via cannula 2.0 M trimethylaluminum in toluene (800 mL). Upon addition of the trimethylaluminum, the mixture started to smoke immediately and gas was evolved. The temperature of the reaction mixture rose to a high of −0.4° C. during the addition, and the addition took a total of about 60 minutes. After all of the trimethylaluminum was added, the mixture was allowed to stir at 20° C. for 3 hours. To the mixture was added 2-methoxybenzonitrile (107 g) as a liquid (previously melted in bath at about 45° C.). Once the addition was complete, the reaction was heated at 90° C. overnight using a heating mantle controlled by a J-KEM. The reaction flask was fitted with a vigreux condenser. Thin-laver chromatography in 50% ethyl acetate/heptane indicated a major baseline product. The reaction mixture was cooled to −8.7° C. in an ice/methanol bath, and to the cold mixture was added 4 L of methanol dropwise via an addition funnel. The addition evolved gas and was exothermic. The temperature of the reaction mixture reached a high of 7.9° C., and the addition took a total of about one hour. After all the methanol was added, the mixture was allowed to stir for three hours at 20° C. The reaction mixture was filtered through filter paper on a benchtop filter. The material collected was washed with additional methanol (2 L). The filtrate was concentrated. The crude material was mixed with 500 mL of ethyl acetate. The mixture was sonicated for 30 minutes and was stirred for another 30 minutes. The material was filtered off and washed with additional ethyl acetate. The material was air dried for an hour and dried under high vacuum for two hours to provide the title compound. ¹H NMR (400 MHz, DMSO-d₆) δ ppm 9.23 (bs, 2H), 7.69 (bs, 1H), 7.63 (ddd, 1H), 7.55 (dd, 1H), 7.25 (dd, 1H), 7.12 (td, 1H), 3.87 (s, 3H). MS (DCI) m=151.0 (M+H)⁺.

### Example 4E

### 4-(dimethoxymethyl)-2-(2-methoxyphenyl)pyrimidine

A dried 5 L three neck flask equipped with a mechanical stirrer, nitrogen inlet into a reflux condenser and outlet to a bubbler, and an internal temperature probe (J-KEM controlled) was charged with Example 4D (126.9 g) and (E)-4-(dimethylamino)-1,1-dimethoxybut-3-en-2-one (177 g). The starting materials were mixed with anhydrous methanol (1360 mL). To the mixture at room temperature under nitrogen was added solid sodium methoxide (257 g) in portions over 20 minutes. The temperature of the reaction went up from 18.6° C. to 35.7° C. during the addition. Once the exotherm was completed, the reaction mixture was heated to 65° C. overnight. LC/MS indicated a single peak corresponding to desired product. The reaction mixture was cooled, and the solvents were concentrated. The residue was mixed with ethyl acetate (800 mL), and water (1 L) was added carefully. The two-phase mixture was sonicated for about 30 minutes to dissolve all the material. The layers were separated, and the organic layer was washed with saturated aqueous NH₄Cl mixture. The combined aqueous extracts were extracted one time with ethyl acetate. The combined organic extracts were washed with brine, dried with Na₂SO₄, filtered, and concentrated. The residue was dissolved in a small amount of dichloromethane (30 mL) and loaded onto a 2.0 L plug of silica in a 3 L Buchner funnel that had been equilibrated with 40% ethyl acetate/heptane. The desired product was eluted with 40% to 50% ethyl acetate/heptane. The pure fractions were combined, and concentrated to provide the title compound. ¹H NMR (500 MHz, DMSO-d₆) δ ppm 8.93 (d, 1H), 7.54 (dd, 1H), 7.50-7.43 (m, 2H), 7.16 (dd, 1H), 7.06 (td, 1H), 5.31 (s, 1H), 3.76 (s, 3H), 3.38 (s, 6H). MS (DCI) m=261.0 (M+H)⁺.

### Example 4F

### (2-(2-methoxyphenyl)pyrimidin-4-yl)methanol

A mixture of Example 4E (14.7 g) in 110 mL HCl in dioxane (4M mixture) and 110 mL water was heated at 50° C. for 14 hours. The mixture was cooled to 0° C., and ground NaOH (17.60 g) was added in portions. The pH was adjusted to 8 using 10% K₂CO₃ aqueous mixture. NaBH₄ (4.27 g) was added in portions. The mixture was stirred at 0° C. for 45 minutes. The mixture was carefully quenched with 150 mL saturated aqueous NH₄Cl and was stirred at 0° C. for 30 minutes. The mixture was extracted with ethyl acetate (5×150 mL), washed with brine, dried over MgSO₄, filtered, and concentrated. The residue was triturated in 30 mL ethanol to give a first crop of the title compound.

The filtrate was concentrated and the residue was purified on a silica gel column (120 g, 55-100% ethyl acetate in heptanes, dry load) to give a second crop of the title compound. ¹H NMR (500 MHz, DMSO-d₆) δ ppm 8.84 (d, 1H), 7.49 (m, 2H), 7.44 (ddd, 1H), 7.13 (dd, 1H), 7.04 (td, 1H), 5.65 (t, 1H), 4.60 (dd, 2H), 3.75 (s, 3H). MS (DCI) m/z 217.0 (M+H)⁺.

### Example 4G

### (R)-ethyl 2-acetoxy-3-(5-((tert-butyldimethylsilyl)oxy)-2-((2-(2-methoxyphenyl)pyrimidin-4-yl)methoxy)phenyl)propanoate

To an oven dried 500 mL round bottom flask was added Example 1D (8 g), triphenylphosphine (13.71 g), Example 4F (6.78 g) and tetrahydrofuran (105 mL). The reaction flask was cooled in an ice bath. Solid (E)-N,N,N′,N′-tetramethyldiazene-1,2-dicarboxamide (9 g) was added, and the reaction mixture was allowed to warm up to ambient temperature and was stirred overnight. After 48 hours, thin-layer chromatography indicated complete consumption of starting material. The reaction mixture was concentrated. Ethyl acetate (50 mL) was added, and the mixture was stirred for about 30 minutes and filtered. The filtrate was concentrated and purified by silica gel chromatography on a Grace Reveleris system using a 120 g silica column with 0-25% ethyl acetate/heptanes. Fractions containing the title compound were combined and concentrated to obtain the title compound. ¹H NMR (400 MHz, DMSO-d₆) δ ppm 8.92 (d, 1H), 7.59-7.50 (m, 2H), 7.46 (ddd, 1H), 7.15 (dd, 1H), 7.05 (td, 1H), 6.95 (d, 1H), 6.77-6.68 (m, 2H), 5.25-5.11 (m, 3H), 4.07 (qd, 2H), 3.76 (s, 3H), 3.26 (dd, 2H), 3.05 (dd, 1H), 1.99 (s, 3H), 1.10 (t, 3H), 0.93 (s, 9H), 0.15 (s, 6H). MS (ESI) m/z 581.4 (M+H)⁺.

### Example 4H

### (R)-ethyl 3-(5-((tert-butyldimethylsilyl)oxy)-2-((2-(2-methoxyphenyl)pyrimidin-4-yl)methoxy)phenyl)-2-hydroxypropanoate

To a mixture of Example 4G (12.60 g) in anhydrous ethanol (220 mL) was added anhydrous potassium carbonate (11.99 g), and the mixture was stirred at room temperature and monitored by LC/MS. After 1 hour, LC/MS showed complete consumption of starting material with a major peak consistent with desired product. The mixture was filtered, and the material was rinsed with ethyl acetate. The filtrate was concentrated under reduced pressure. To the residue was added water (100 mL) and ethyl acetate (100 mL). The layers were separated, and the aqueous layer was extracted with three portions of ethyl acetate. The combined organic layers were dried over anhydrous sodium sulfate, filtered and concentrated. The crude product was used in the next step without further purification. LC/MS (APCI) m/z 539.2 (M+H)⁺.

### Example 41

### (R)-ethyl 2-((5-bromo-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 4H (11.10 g) and Example 1L (7.08 g) was added anhydrous cesium carbonate (20.14 g). The mixture was evacuated and backfilled with nitrogen, and anhydrous tert-butanol (180 mL) was added. The mixture was stirred at 65° C. for 5 hours and was concentrated under reduced pressure. The residue was diluted with ethyl acetate, washed with water and brine, dried over anhydrous sodium sulfate, filtered, and concentrated. The crude material was purified by silica gel chromatography on an AnaLogix IntelliFlash²⁸⁰ system (10-70% ethyl acetate/heptanes, linear gradient) to provide the title compound. LC/MS (APCI) m/z 847.1 (M+H)⁺.

### Example 4J

### (R)-ethyl 2-(((S)-5-((1S)-4-(((R)-1-(bis(4-methoxyphenyl) (phenyl)methoxy)-3-(tosyloxy) propan-2-yl)oxy)-3-chloro-2-methylphenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

The title compound was prepared using the conditions described in Example 1U, substituting Example 4I for Example 1M. ¹H NMR (400 MHz, DMSO-d₆) δ ppm 0.02-0.06 (m, 6H), 0.86 (s, 9H), 0.93 (t, 3H), 1.97 (s, 3H), 2.26-2.32 (m, 1H), 2.35 (s, 3H), 2.40-2.47 (m, 1H), 2.73 (dd, 1H), 3.08-3.26 (m, 2H), 3.64 (d, 6H), 3.73 (s, 3H), 3.86-3.99 (m, 1H), 4.15-4.30 (m, 2H), 4.67-4.78 (m, 1H), 5.04-5.09 (m, 2H), 5.55 (t, 1H), 6.22 (d, 1H), 6.65 (td, 1H), 6.70-6.76 (m, 3H), 6.84-6.95 (m, 2H), 7.01 (td, 1H), 7.08-7.32 (m, 11H), 7.31-7.41 (m, 4H), 7.41-7.60 (m, 2H), 7.63-7.70 (m, 2H), 8.60 (s, 1H), 8.80 (d, 1H).

### Example 4K

### (R)-ethyl 2-(((S)-5-((1S)-4-(((R)-1-(bis(4-methoxyphenyl) (phenyl)methoxy)-3-(tosyloxy) propan-2-yl)oxy)-3-chloro-2-methylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

Example 4J (1.76 g) was dissolved in dichloromethane (61.2 mL) and was treated with tetrabutylammonium fluoride (1.224 mL, 1 M in tetrahydrofuran) at ambient temperature for 15 minutes. The mixture was concentrated onto silica gel and purification by flash chromatography on a CombiFlash® Teledyne Isco system using a Teledyne Isco RediSep® Rf gold 80 g silica gel column (eluting with 10-100% ethyl acetate/heptane) provided the title compound. ¹H NMR (400 MHz, DMSO-d₆) δ ppm 1.00 (t, 3H), 1.93 (s, 3H), 2.35 (s, 3H), 2.71 (dd, 1H), 3.09 (dd, 1H), 3.24 (dd, 1H), 3.65 (d, 6H), 3.73 (s, 3H), 3.95-4.07 (m, 2H), 4.19-4.35 (m, 2H), 4.72-4.86 (m, 1H), 4.97-5.09 (m, 2H), 5.40 (dd, 1H), 5.93 (d, 1H), 6.56 (dd, 1H), 6.69-6.77 (m, 4H), 6.78-6.85 (m, 2H), 6.88-6.95 (m, 1H), 7.01 (td, 1H), 7.05-7.28 (m, 12H), 7.31-7.40 (m, 4H), 7.41-7.47 (m, 2H), 7.50 (dd, 1H), 7.66-7.75 (m, 2H), 8.59 (s, 1H), 8.81 (s, 1H), 8.83 (d, 1H).

### Example 4L

### ethyl (7R,16S,21S)-16-{[bis(4-methoxyphenyl) (phenyl)methoxy]methyl}-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 4K (535 mg) in N,N-dimethylformamide (53.9 mL) was added cesium carbonate (1317 mg). The reaction mixture was stirred at 40° C. for 2 hours. The mixture was cooled to ambient temperature, poured into a separatory funnel, and diluted with ethyl acetate and water. The layers were separated, and the aqueous layer was extracted with two portions of ethyl acetate. The combined organics were washed with brine, dried over anhydrous magnesium sulfate, filtered and concentrated onto silica gel. Purification by silica gel chromatography on a CombiFlash® Teledyne Isco system using a Teledyne Isco RediSep® Rf gold 40 g silica gel column (eluting with 20-100% ethyl acetate/heptane) provided the title compound. LC/MS (APCI) m/z 1151.1 (M+H)⁺.

### Example 4M

### ethyl (7R,16R,21S)-19-chloro-1-(4-fluorophenyl)-16-(hydroxymethyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

Example 4L (350 mg) was treated with a mixture of methanol (1.5 mL), dichloromethane (1.5 mL) and formic acid (1.5 mL) for 15 minutes. The mixture was then carefully poured into 50 mL of saturated aqueous sodium bicarbonate and extracted with three portions of ethyl acetate. The combined organic layers were washed with saturated aqueous brine, dried over anhydrous magnesium sulfate, filtered, and concentrated onto silica gel. Purification by silica chromatography on a CombiFlash® Teledyne Isco system using a Teledyne Isco RediSep® Rf gold 24 g silica gel column (eluting with 20-100% ethyl acetate/heptane) provided the title compound. LC/MS (APCI) m/z 849.3 (M+H)⁺.

### Example 4N

### ethyl (7R,16S,21S)-19-chloro-1-(4-fluorophenyl)-10-{[2-(2-methoxyphenyl)pyrimidin-4-yl]methoxy}-20-methyl-16-{[(4-methylbenzene-1-sulfonyl)oxy]methyl}-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 4M (183 mg) and triethylamine (90 μL) in dichloromethane (2.2 mL) was added para-toluenesulfonyl chloride (82 mg) in one portion. The mixture was stirred at ambient temperature overnight. The mixture was concentrated onto silica gel and purification by flash chromatography on a CombiFlash® Teledyne Isco system using a Teledyne Isco RediSep® Rf gold 24 g silica gel column (eluting with 20-100% ethyl acetate/heptane) provided the title compound. LC/MS (APCI) m/z 1003.1 (M+H)⁺.

### Example 40

### ethyl (7R,16R,21S)-19-chloro-1-(4-fluorophenyl)-10-{[2-(2-methoxyphenyl)pyrimidin-4-yl]methoxy}-20-methyl-16-{[4-(2,5,8,11-tetraoxatridecan-13-yl) piperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylate

A 2.0 mL vial was charged with Example 4N (180 mg), Example 4C (317 mg), dimethylformamide (0.4 mL) and triethylamine (0.160 mL). The vial was capped and stirred at 45° C. for 1 day. The mixture was diluted with ethyl acetate and washed with water. The organics were dried over MgSO₄, filtered, and concentrated in vacuo. The residue was purified by silica gel flash chromatography on AnaLogix IntelliFlash²⁸⁰ system eluting with 2-10% methanol in CH₂Cl₂ to provide the title compound. MS (ESI) m/z 1107.5 (M+H)⁺.

### Example 4P

### (7R,16R,21S)-19-chloro-1-(4-fluorophenyl)-10-{[2-(2-methoxyphenyl)pyrimidin-4-yl]methoxy}-20-methyl-16-{[4-(2,5,8,11-tetraoxatridecan-13-yl) piperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylic Acid

To a mixture of Example 40 (170 mg) in tetrahydrofuran (1.50 mL) and methanol (0.75 mL) at 0° C. was slowly added lithium hydroxide mixture (1.0 M in H₂O, 1.228 mL). The mixture was stirred at ambient temperature for 1 day. The reaction mixture was concentrated, and was dissolved in DMSO-H₂O (4/1) (1 mL) and acidified with acetic acid. The mixture was purified on a Gilson prep HPLC (Zorbax, C-18, 250×21.2 mm column, 5-75% acetonitrile in water (0.1% TFA)) to provide the title compound after lyophilization. ¹H NMR (500 MHz, DMSO-d₆) δ ppm 8.89 (d, 1H), 8.75 (d, 1H), 7.57-7.51 (m, 2H), 7.47 (ddd, 1H), 7.24-7.13 (m, 6H), 7.06 (td, 1H), 6.97 (d, 1H), 6.91 (d, 1H), 6.84 (dd, 1H), 6.16 (dd, 1H), 5.67 (d, 1H), 5.26-5.08 (m, 2H), 4.70-4.40 (m, 6H), 3.87 (dd, 1H), 3.77 (s, 3H), 3.74 (t, 2H), 3.61-3.39 (m, 14H), 3.29 (s, 2H), 3.22 (s, 3H), 3.18-2.70 (m, 6H), 2.23 (s, 3H). MS (ESI) m/z 1079.2 (M+H)⁺.

### Example 5

### (7R,16R,21S)-19-chloro-1-(4-fluorophenyl)-16-[(4-{2-[2-(2-methoxyethoxy)ethoxy]ethyl}piperazin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 5A

### 2-(2-(2-methoxyethoxy)ethoxy)ethyl 4-methylbenzenesulfonate

The title compound was prepared using the conditions described in Example 4A substituting 2-(2-(2-methoxyethoxy)ethoxy)ethanol for 3,6,9,12-tetraoxatetradecan-1-ol. MS (ESI) m/z 319.0 (M+H)⁺.

### Example 5B

### tert-butyl 4-(2-(2-(2-methoxyethoxy)ethoxy)ethyl)piperazine-1-carboxylate

The title compound was prepared using the conditions described in Example 4B, substituting Example 5A for Example 4A. MS (ESI) m/z 333.2 (M+H)⁺.

### Example 5C

### 1-(2-(2-(2-methoxyethoxy)ethoxy)ethyl)piperazine

The title compound was prepared using the conditions described in Example 4C, substituting Example 5B for Example 4B. MS (ESI) m/z 233.3 (M+H)⁺.

### Example 5D

### ethyl (7R,16R,21S)-19-chloro-1-(4-fluorophenyl)-16-[(4-{2-[2-(2-methoxyethoxy)ethoxy]ethyl}piperazin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared using the conditions described in Example 40, substituting Example 5C for Example 4C. MS (ESI) m/z 1063.3 (M+H)⁺.

### Example 5E

### (7R,16R,21S)-19-chloro-1-(4-fluorophenyl)-16-[(4-{2-[2-(2-methoxyethoxy)ethoxy]ethyl}piperazin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared using the conditions described in Example 4P, substituting Example 5D for Example 40. ¹H NMR (501 MHz, DMSO-d₆) δ ppm 8.89 ppm (d, 1H), 8.75 (s, 1H), 7.57-7.51 (m, 2H), 7.47 (ddd, 1H), 7.24-7.12 (m, 6H), 7.06 (td, 1H), 6.97 (d, 1H), 6.91 (d, 1H), 6.84 (dd, 1H), 6.16 (dd, 1H), 5.67 (d, 1H), 5.25-5.10 (m, 2H), 4.70-3.90 (m, 6H), 3.87 (dd, 1H), 3.77 (s, 3H), 3.74 (t, 2H), 3.60-3.37 (m, 10H), 3.29 (s, 2H), 3.20 (s, 3H), 3.17-2.71 (m, 6H), 2.23 (s, 3H). MS (ESI) m/z 1035.5 (M+H)⁺.

### Example 6

### methyl 6-(4-{[(7R,16R,21S)-7-carboxy-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]inden-16-yl]methyl}piperazin-1-yl)-6-deoxy-2,3,4-tri-O-methyl-a-D-mannopyranoside

### Example 6A

### (2S,3S,4S,5S,6R)-2-methoxy-6-((trityloxy)methyl)tetrahydro-2H-pyran-3,4,5-triol

To a mixture of (2R,3S,4S,5S,6S)-2-(hydroxymethyl)-6-methoxytetrahydro-2H-pyran-3,4,5-triol (25 g) in pyridine (150 mL) was added trityl chloride (39.5 g) at 25° C. The reaction was stirred at 40° C. for 5 hours. The reaction was cooled to 20° C. and was concentrated under reduced pressure to give a residue which was purified by column chromatography on silica gel (eluting with petroleum ether:ethyl acetate 50:1-1:1) to provide the title compound. ¹H NMR (400 MHz, CDCl₃) δ ppm 7.47 (d, 6H), 7.36-7.23 (m, 9H), 4.70 (br d, 1H), 3.86 (br d, 1H), 3.75 (br s, 1H), 3.68 (br d, 2H), 3.43 (br s, 2H), 3.39 (s, 3H), 3.33-2.48 (m, 3H).

### Example 6B

### (2S,3S,4S,5R,6R)-2,3,4,5-tetramethoxy-6-((trityloxy)methyl)tetrahydro-2H-pyran

To a mixture of Example 6A (35 g) in dimethylformamide (500 mL) was added NaH (12.51 g, 60% in mineral oil) at 0° C. The reaction was stirred at 0° C. for 1 hour. Methyl iodide (22.56 mL) was added slowly at 0° C. The reaction was stirred at 25° C. for 10 hours. The reaction mixture was diluted with water (500 mL) and extracted with ethyl acetate (3×400 mL). The combined organic layers were washed with brine (3×250 mL) and dried over Na₂SO₄. After filtering, the filtrate was concentrated under reduced pressure to give a residue which was washed with petroleum ether (250 ml). The material was collected by suction filtration to provide the title compound. ¹H NMR (400 MHz, CDCl₃) δ ppm 7.43 (d, 6H), 7.24-7.10 (m, 9H), 4.79 (d, 1H), 3.56-3.50 (m, 2H), 3.45 (s, 3H), 3.43-3.38 (m, 5H), 3.37 (s, 3H), 3.31 (dd, 1H), 3.18 (s, 3H), 3.11 (dd, 1H).

### Example 6C

### ((2R,3R,4S,5S,6S)-3,4,5,6-tetramethoxytetrahydro-2H-pyran-2-yl)methanol

To a mixture of Example 6B (18 g) in acetic acid (300 mL) was added water (150 mL) at 20° C. The reaction was stirred at 90° C. for 1 hour. The reaction mixture was cooled to 30° C., poured into ice water (250 mL) and filtered. The filtrate was extracted with ethyl acetate (3×250 mL) and the combined organic layers were washed with brine (3×150 mL). The organic layer was dried over Na₂SO₄ and filtered. The filtrate was concentrated to provide the title compound. ¹H NMR (400 MHz, CDCl₃) δ ppm 4.76 (s, 1H), 3.85-3.79 (m, 1H), 3.77-3.70 (m, 1H), 3.56 (br d, 1H), 3.54-3.52 (m, 3H), 3.48 (s, 8H), 3.46-3.41 (m, 1H), 3.37-3.34 (m, 3H).

### Example 6D

### ((2R,3R,4S,5S,6S)-3,4,5,6-tetramethoxytetrahydro-2H-pyran-2-yl)methyl 4-methylbenzenesulfonate

The title compound was prepared using the conditions described in Example 4A substituting Example 6C for 3,6,9,12-tetraoxatetradecan-1-ol. LC/MS (APCI) m/z 408.3 (M+NH₄)⁺.

### Example 6E

### tert-butyl 4-(((2R,3R,4S,5S,6S)-3,4,5,6-tetramethoxytetrahydro-2H-pyran-2-yl)methyl)piperazine-1-carboxylate

The title compound was prepared using the conditions described in Example 4B, substituting Example 6D for Example 4A. MS (ESI) m/z 405.2 (M+H)⁺.

### Example 6F

### 1-(2R,3R,4S,5S,6S)-3,4,5,6-tetramethoxytetrahydro-2H-pyran-2-yl)methyl)piperazine

The title compound was prepared using the conditions described in Example 4C, substituting Example 6E for Example 4B. MS (ESI) m/z 305.3 (M+H)⁺.

### Example 6G

### methyl 6-(4-{[(7R,16R,21S)-19-chloro-7-(ethoxycarbonyl)-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]inden-16-yl]methyl}piperazin-1-yl)-6-deoxy-2,3,4-tri-O-methyl-a-D-mannopyranoside

The title compound was prepared using the conditions described in Example 40, substituting Example 6F for Example 4C. MS (ESI) m/z 1135.5 (M+H)⁺.

### Example 6H

### methyl 6-(4-{[(7R,16R,21S)-7-carboxy-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]inden-16-yl]methyl}piperazin-1-yl)-6-deoxy-2,3,4-tri-O-methyl-a-D-mannopyranoside

The title compound was prepared using the conditions described in Example 4P, substituting Example 6G for Example 40. ¹H NMR (400 MHz, DMSO-d₆) δ ppm 8.89 (d, 1H), 8.75 (s, 1H), 7.57-7.52 (m, 2H), 7.51-7.43 (m, 1H), 7.19 (dtd, 6H), 7.06 (t, 1H), 6.97 (d, 1H), 6.91 (d, 1H), 6.85 (dd, 1H), 6.16 (dd, 1H), 5.68 (d, 1H), 5.17 (q, 2H), 4.81 (d, 1H), 4.66 (s, 1H), 4.51-4.31 (m, 2H), 3.91-3.80 (m, 1H), 3.77 (s, 3H), 3.64-3.60 (m, 1H), 3.42 (s, 3H), 3.41-3.37 (m, 11H), 3.36 (s, 3H), 3.35 (s, 3H), 3.34 (s, 3H), 3.15 (t, 2H), 2.96-2.87 (m, 2H), 2.23 (s, 3H). MS (ESI) m/z 1035.5 (M+H)⁺.

### Example 7

### methyl 6-O-{3-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

### Example 7A

### 4,4,5,5-tetramethyl-2-(3-(((2R,3R,4S,5S,6S)-3,4,5,6-tetramethoxytetrahydro-2H-pyran-2-yl)methoxy)phenyl)-1,3,2-dioxaborolane

To a mixture of Example 6C (10.4 g), 3-(4,4,5,5-tetramethyl-1,3,2-dioxaborolan-2-yl)phenol (25.2 g), and triphenylphosphine (18.47 g) in toluene (200 mL) was added (E)-di-tert-butyl diazene-1,2-dicarboxylate (12.16 g) at 20° C. The reaction was stirred at 70° C. for 10 hours. The reaction mixture was concentrated under reduced pressure to give a residue which was purified by column chromatography on silica gel (petroleum ether:ethyl acetate 100:1-50:1) to provide the title compound. ¹H NMR (400 MHz, CDCl₃) δ ppm 7.43-7.37 (m, 2H), 7.32-7.28 (m, 1H), 7.10-7.05 (m, 1H), 4.85 (s, 1H), 4.28-4.18 (m, 2H), 4.28-4.18 (m, 1H), 3.81-3.74 (m, 1H), 3.64 (m, 1H), 3.60 (br s, 1H), 3.57 (br d, 1H), 3.52 (s, 6H), 3.50 (s, 3H), 3.40 (s, 3H), 1.35 (s, 12H).

### Example 7B

### (2-(3-(((2R,3R,4S,5S,6S)-3,4,5,6-tetramethoxytetrahydro-2H-pyran-2-yl)methoxy)phenyl)pyrimidin-4-yl)methanol

A stirring mixture of 2-chloropyrimidine-4-yl)methanol (1.25 g), Example 7A (4.17 g), and tetrakis(triphenylphosphine)palladium (0.999 g) in tetrahydrofuran (55.0 mL) and saturated sodium bicarbonate in water (31.4 mL) was degassed by bubbling nitrogen through the mixture via a syringe needle for 10 minutes. The mixture was stirred under nitrogen at 75° C. for 15 hours. After cooling to ambient temperature, the mixture was diluted with saturated aqueous sodium bicarbonate (50 mL). The mixture was extracted with three 40 mL portions of ethyl acetate. The combined organic layers were dried over anhydrous magnesium sulfate, filtered and concentrated onto silica gel. Purification by flash chromatography on a CombiFlash® Teledyne Isco system using a Teledyne Isco RediSep® Rf gold 80 g silica gel column (eluting with 30-100% ethyl acetate/heptanes) provided the title compound. LC/MS (APCI) m/z 421.3 (M+H)⁺.

### Example 7C

### (R)-ethyl 2-acetoxy-3-(5-((tert-butyldimethylsilyl)oxy)-2-((2-(3-(((2R,3R,4S,5S,6S)-3,4,5,6-tetramethoxytetrahydro-2H-pyran-2-yl)methoxy)phenyl)pyrimidin-4-yl)methoxy)phenyl)propanoate

A mixture of N,N,N′,N′-tetramethylazodicarboxylate (0.900 g) and triphenylphosphine (1.371 g) was stirred in 13 mL of tetrahydrofuran at 0° C. for 20 minutes. The mixture was added to a separate flask containing Example 1D (1.0 g) and Example 7B (1.43 g) cooled in an ice bath. The resulting reaction mixture was stirred for 1 hour at 0° C. The cooling bath was removed and the mixture was stirred for 16 hours. The mixture was concentrated onto silica gel, and purification by flash chromatography on a CombiFlash® Teledyne Isco system using a Teledyne Isco RediSep® Rf gold 40 g silica gel column (eluting with 10-70% ethyl acetate/heptanes) provided the title compound. LC/MS (APCI) m/z 785.3 (M+H)⁺.

### Example 7D

### (R)-ethyl 3-(5-((tert-butyldimethylsilyl)oxy)-2-((2-(3-(((2R,3R,4S,5S,6S)-3,4,5,6-tetramethoxytetrahydro-2H-pyran-2-yl)methoxy)phenyl)pyrimidin-4-yl)methoxy)phenyl)-2-hydroxypropanoate

To a mixture of Example 7C (1.56 g) in 13 mL of ethanol was added 1.1 g of anhydrous potassium carbonate and the mixture was stirred at room temperature for 10 hours. The mixture was poured into 80 mL of water and the mixture was extracted with three portions of ethyl acetate. The combined organic layers were dried over anhydrous magnesium sulfate, filtered and concentrated onto silica gel. Purification by flash chromatography on a CombiFlash® Teledyne Isco system using a Teledyne Isco RediSep® Rf gold 80 g silica gel column (eluting with 10-80% ethyl acetate/heptanes) provided the title compound. LC/MS (APCI) m/z 743.0 (M+H)⁺.

### Example 7E

### (R)-ethyl 2-((5-bromo-6-(4-fluorophenyl)thieno[2,3-d]pyrimidin-4-yl)oxy)-3-(5-((tert-butyldimethylsilyl)oxy)-2-((2-(3-(((2R,3R,4S,5S,6S)-3,4,5,6-tetramethoxytetrahydro-2H-pyran-2-yl)methoxy)phenyl)pyrimidin-4-yl)methoxy)phenyl)propanoate

A mixture of Example 7D (1100 mg), Example 1L (509 mg) and cesium carbonate (1447 mg) was evacuated and backfilled with N₂. Anhydrous tert-butanol (12 mL) was added and the mixture was stirred at 65° C. for 3 hours. The reaction mixture was concentrated in vacuo and was diluted with ethyl acetate. The mixture was washed with water and saturated brine, dried over anhydrous sodium sulfate, filtered, and concentrated. The residue was purified by silica gel flash chromatography on AnaLogix IntelliFlash²⁸⁰ system (10-70% ethyl acetate/hexanes, linear gradient) to provide the title compound. MS (ESI) m/z 1051.1 (M+H)⁺.

### Example 7F

### (2R)-ethyl 2-((5-((1S)-4-(((R)-1-(bis(4-methoxyphenyl) (phenyl)methoxy)-3-(tosyloxy) propan-2-yl)oxy)-3-chloro-2-methylphenyl)-6-(4-fluorophenyl)thieno[2,3-d]pyrimidin-4-yl)oxy)-3-(5-((tert-butyldimethylsilyl)oxy)-2-((2-(3-(((2R,3R,4S,5S,6S)-3,4,5,6-tetramethoxytetrahydro-2H-pyran-2-yl)methoxy)phenyl)pyrimidin-4-yl)methoxy)phenyl)propanoate

A 100 mL flask, equipped with stir bar and septa, was charged with Example 7E (1240 mg), Example 1T (1227 mg), bis(di-tert-butyl (4-dimethylaminophenyl)phosphine)dichloropalladium (II) (84 mg) and cesium carbonate (1154 mg). The flask was capped, evacuated and backfilled with nitrogen twice. Freshly degassed tetrahydrofuran (5.0 mL) followed by water (1.25 mL) were introduced and the reaction mixture was evacuated and backfilled with nitrogen twice again while stirring. The mixture was stirred at 40° C. for 16 hours. The reaction mixture was diluted with ethyl acetate and water. The organic layer was collected and the aqueous layer was extracted with two portions of ethyl acetate. The organic layers were combined, dried over anhydrous magnesium sulfate, filtered, and concentrated. The residue was purified by silica gel flash chromatography on AnaLogix IntelliFlash²⁸⁰ system (20-80% ethyl acetate/hexanes, linear gradient) to provide the title compound. LC/MS (ESI) m/z 1643.2 (M+H)⁺.

### Example 7G

### (2R)-ethyl 2-((5-((1S)-4-(((R)-1-(bis(4-methoxyphenyl) (phenyl)methoxy)-3-(tosyloxy) propan-2-yl)oxy)-3-chloro-2-methylphenyl)-6-(4-fluorophenyl)thieno[2,3-d]pyrimidin-4-yl)oxy)-3-(5-hydroxy-2-((2-(3-(((2R,3R,4S,5S,6S)-3,4,5,6-tetramethoxytetrahydro-2H-pyran-2-yl)methoxy)phenyl)pyrimidin-4-yl)methoxy)phenyl)propanoate

To a mixture of Example 7F (1580 mg) in CH₂Cl₂ (45 mL) was added tetrabutylammonium fluoride mixture (1.0 M in tetrahydrofuran, 0.962 mL). The mixture was stirred for 40 minutes. The reaction mixture was concentrated in vacuo. The residue was purified by silica gel flash chromatography on an AnaLogix IntelliFlash²⁸⁰ system (30-80% ethyl acetate/hexanes, linear gradient) to provide the title compound. MS (ESI) m/z 1549.0 (M+Na)⁺.

### Example 7H

### methyl 6-O-{3-[4-({[(7R,16S,21S)-16-{[bis(4-methoxyphenyl)(phenyl)methoxy]methyl}-19-chloro-7-(ethoxycarbonyl)-1-(4-fluorophenyl)-20-methyl-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

To Example 7G (1100 mg) in dimethyl formamide (70 mL) was added cesium carbonate (2345 mg). The reaction mixture was stirred for 5 hours. The reaction mixture was diluted with ethyl acetate and water. The organic layer was collected and the aqueous layer was extracted with two portions of ethyl acetate. The organic layers were combined, dried over anhydrous magnesium sulfate, filtered and concentrated. The residue was purified by silica gel flash chromatography on an AnaLogix IntelliFlash²⁸⁰ system (30-80% ethyl acetate/hexanes, linear gradient) to provide the title compound. MS (ESI) m/z 1355.3 (M+H)⁺.

### Example 7I

### methyl 6-O-{3-[4-({[(7R,16R,21S)-19-chloro-7-(ethoxycarbonyl)-1-(4-fluorophenyl)-16-(hydroxymethyl)-20-methyl-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

To a mixture of Example 7H (700 mg) in CH₂Cl₂ (2.80 mL) and methanol (2.80 mL) was added formic acid (2281 mg). The reaction mixture was stirred at room temperature for 30 minutes. The reaction mixture was carefully added dropwise into saturated aqueous NaHCO₃. The resulting mixture was extracted twice with ethyl acetate. The combined organics were washed with brine, dried over Na₂SO₄, filtered, and concentrated. The residue was purified by silica gel flash chromatography on an AnaLogix IntelliFlash²⁸⁰ system (70-100% ethyl acetate/heptanes, linear gradient) to provide the title compound. MS (ESI) m/z 1053.3 (M+H)⁺.

### Example 7J

### methyl 6-O-{3-[4-({[(7R,16S,21S)-19-chloro-7-(ethoxycarbonyl)-1-(4-fluorophenyl)-20-methyl-16-{[(4-methylbenzene-1-sulfonyl)oxy]methyl}-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

To a mixture of Example 71 (400 mg) in CH₂Cl₂ (4 mL) was added triethylamine (92 mg) and p-toluenesulfonyl chloride (116 mg). The reaction mixture was stirred at room temperature for 1 day. The mixture was purified by silica gel flash chromatography on an AnaLogix IntelliFlash²⁸⁰ system (50-100% ethyl acetate/heptanes, linear gradient) to provide the title compound. MS (ESI) m/z 1207.0 (M+H)⁺.

### Example 7K

### methyl 6-O-{3-[4-({[(7R,16R,21S)-19-chloro-7-(ethoxycarbonyl)-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

A 4 mL vial was charged with Example 7J (100 mg), 1-methylpiperazine (199 mg) and dimethylformamide (0.27 mL). The vial was capped and stirred at 45° C. for 8 hours. To the mixture was added 2 mL of water. The precipitate obtained was sonicated for a few minutes, and filtered and washed with 2 ml of water. The material was collected and dried under high vacuum to provide the title compound. LC/MS (ESI) m/z 1135.5 (M+H)⁺.

### Example 7L

### methyl 6-O-{3-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

To a mixture of Example 7K (90 mg) in tetrahydrofuran (0.64 mL) and methanol (0.320 mL) was slowly added LiOH (1.0 M in H₂O, 0.634 mL). The mixture was stirred for 16 hours. The reaction mixture was acidified at 0° C. with acetic acid. The mixture was purified on Gilson prep HPLC (Zorbax, C-18, 250×21.2 mm column, 5-75% acetonitrile in water (0.1% TFA)) followed by silica gel thin-layer chromatography (eluent: methanol/CH₂Cl₂ (1/8)) to provide the title compound. ¹H NMR (501 MHz, DMSO-d₆) δ ppm 8.91 (d, 1H), 8.75 (s, 1H), 8.06-7.95 (m, 2H), 7.53 (d, 1H), 7.47 (t, 1H), 7.23-7.12 (m, 6H), 6.94 (dd, 2H), 6.83 (dd, 1H), 6.17 (dd, 1H), 5.67 (d, 1H), 5.33-5.15 (m, 2H), 4.79 (d, 1H), 4.58 (q, 1H), 4.47 (d, 1H), 4.36 (dd, 1H), 4.21 (qd, 2H), 3.89 (dd, 1H), 3.68-3.59 (m, 2H), 3.53-3.41 (m, 6H), 3.39 (s, 3H), 3.38 (s, 3H), 3.36 (s, 3H), 3.30 (s, 3H), 3.16-2.87 (m, 4H), 2.79 (s, 3H), 2.74 (t, 2H), 2.22 (s, 3H). MS (ESI) m/z 1107.8 (M+H)⁺.

### Example 8

### methyl 6-O-{3-[4-({[(7S,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

The title compound was isolated as a minor product during the synthesis of Example 7J. ¹H NMR (400 MHz, DMSO-d₆) δ ppm 8.90 (d, 1H), 8.69-8.53 (m, 1H), 7.98 (t, 2H), 7.71 (s, 1H), 7.44 (t, 1H), 7.17 (dd, 5H), 6.89 (d, 2H), 6.69 (d, 2H), 5.94 (s, 1H), 5.22 (d, 2H), 4.95 (s, 1H), 4.79 (d, 1H), 4.26-3.98 (m, 4H), 3.70-3.54 (m, 2H), 3.49-3.40 (m, 2H), 3.39 (s, 6H), 3.36 (s, 3H), 3.35-3.31 (m, 8H), 3.30 (s, 3H), 3.07-2.56 (m, 7H), 2.27 (s, 3H). MS (ESI) m/z 1107.3 (M+H)⁺.

### Example 9

### methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-glucopyranoside

### Example 9A

### (2S,3R,4S,5R,6R)-2-methoxy-6-((4-(4,4,5,5-tetramethyl-1,3,2-dioxaborolan-2-yl)phenoxy)methyl)tetrahydro-2H-pyran-3,4,5-triyl triacetate

To a mixture of 4-(4,4,5,5-tetramethyl-1,3,2-dioxaborolan-2-yl)phenol (0.400 g), (2R,3R,4S,5R,6S)-2-(hydroxymethyl)-6-methoxytetrahydro-2H-pyran-3,4,5-triyl triacetate (0.873 g) and triphenylphosphine (0.715 g) in toluene (10 mL) was added di-tert-butyl azidicarboxylate (0.628 g).

The reaction was stirred at room temperature. The reaction was stirred for 3 hours at room temperature and heated to 60° C. for an additional 3 hours. The reaction was cooled, loaded directly onto a silica gel column (Teledyne Isco RediSep® Rf gold 80 g) and was eluted using a gradient of 5-75% heptanes/ethyl acetate. The title compound containing fractions were combined and concentrated. The crude material was taken up in diethyl ether and was concentrated to provide the title compound. ¹H NMR (400 MHz, CDCl₃) δ ppm 7.73 (d, 2H), 6.88 (d, 2H), 5.52 (t, 1H), 5.17 (t, 1H), 4.98 (d, 1H), 4.93 (dd, 1H), 4.22-4.11 (m, 1H), 4.12-4.00 (m, 2H), 3.44 (s, 3H), 2.08 (s, 3H), 2.02 (s, 3H), 2.01 (s, 3H), 1.33 (s, 12H). MS (ESI) m/z 540.1 (M+NH₄)⁺.

### Example 9B

### (2R,3R,4S,5R,6S)-2-((4-(4-(hydroxymethyl)pyrimidin-2-yl)phenoxy)methyl)-6-methoxytetrahydro-2H-pyran-3,4,5-triyl triacetate

To a mixture of (2-chloropyrimidin-4-yl)methanol (40 mg), Example 9A (123 mg) and tetrakis(triphenylphosphine)palladium (0) (32.0 mg) in tetrahydrofuran (1.8 mL) was added a mixture of saturated aqueous sodium bicarbonate (1.0 mL). The reaction was flushed with nitrogen and heated to 75° C. overnight. The reaction was cooled, diluted with ethyl acetate (50 mL), and washed with water (25 mL) and brine (25 mL). The organic layer was dried over magnesium sulfate, filtered, and concentrated. The residue was loaded onto silica gel (Teledyne Isco RediSep® Rf gold 24 g) and was eluted using a gradient of 5-85% heptanes/ethyl acetate. The desired product containing fractions were combined to provide the title compound. ¹H NMR (400 MHz, CDCl₃) δ ppm 8.70 (d, 1H), 8.45-8.37 (m, 2H), 7.10 (d, 1H), 7.04-6.97 (m, 2H), 5.54 (t, 1H), 5.20 (t, 1H), 5.00 (d, 1H), 4.95 (dd, 1H), 4.79 (d, 2H), 4.24-4.08 (m, 3H), 3.64 (t, 1H), 3.46 (s, 3H), 2.09 (s, 3H), 2.03 (s, 3H), 2.03 (s, 3H). MS (ESI) m/z 505.1 (M+H)⁺.

### Example 9C

### (2R,3R,4S,5R,6S)-2-((4-(4-(chloromethyl)pyrimidin-2-yl)phenoxy)methyl)-6-methoxytetrahydro-2H-pyran-3,4,5-triyl triacetate

To Example 9B (0.069) in dichloromethane (0.5 mL) was added triphenylphosphine (0.039 g) followed by N-chlorosuccinimide (0.020 g). The reaction was stirred at 0° C. for 1 hour. Additional triphenylphosphine (0.039 g) and N-chlorosuccinimide (0.020 g) was added and stirring was continued for an additional 1 hour at 0° C. The reaction was loaded onto silica gel (Teledyne Isco RediSep® Rf gold 24 g) and was eluted using a gradient of 5-75% heptanes/ethyl acetate. The desired product containing fractions were combined and concentrated to provide the title compound. ¹H NMR (400 MHz, CDCl₃) δ ppm 8.78 (d, 1H), 8.46-8.33 (m, 2H), 7.35 (d, 1H), 7.04-6.93 (m, 2H), 5.54 (dd, 1H), 5.20 (dd, 1H), 5.00 (d, 1H), 4.95 (dd, 1H), 4.65 (s, 2H), 4.23-4.08 (m, 3H), 3.45 (s, 3H), 2.09 (s, 3H), 2.03 (s, 3H), 2.03 (s, 3H). MS (ESI) m/z 523.2 (M+H)⁺.

### Example 9D

### ethyl 2-acetoxy-3-(2-(benzyloxy)phenyl)acrylate

A 2 L three-necked round bottom flask equipped with an internal temperature probe was charged with ethyl 2-acetoxy-2-(diethoxyphosphoryl)acetate (86 g) and anhydrous tetrahydrofuran (1 L) at room temperature under nitrogen. To the mixture was added cesium carbonate (100 g, 307 mmol) in one portion. The reaction mixture was stirred for about 20 minutes, and 2-(benzyloxy)benzaldehyde (50 g) was added as a solid in one portion. The slurry was stirred vigorously overnight at room temperature. Thin-layer chromatography in 10% ethyl acetate/heptane indicted the reaction about 60 to 70% complete. Another 0.5 equivalent of ethyl 2-acetoxy-2-(diethoxyphosphoryl)acetate and cesium carbonate were added, and the reaction was stirred overnight. Thin-layer chromatography indicated the reaction was complete. The reaction mixture was cooled to about 0° C. in an ice bath, and reaction was quenched with the addition of water (500 mL) in portions, such that the temperature of the reaction was maintained below 10° C. The reaction was diluted with ethyl acetate (500 mL), and the mixture was stirred for 30 minutes. The mixture was poured into a separatory funnel and was further diluted with ethyl acetate and water to a total volume of 2.6 L. The organic layer was separated, washed with brine, dried with Na₂SO₄, filtered, and concentrated. The residue was dissolved in 2:1 heptane/dichloromethane and was purified on a 2 L silica gel plug equilibrated with 100% heptane. The material was eluted with 5% to 10% ethyl acetate/heptane. Fractions containing the desired product were combined, and the solvents were removed under reduced pressure to provide the title compound. NMR showed the material was about a 2:1 mix of E and Z isomer. ¹H NMR (501 MHz, DMSO-d₆) δ ppm 7.71 (m, 2H), 7.50-7.25 (m, 12H), 7.20 (dd, 1H), 7.11 (dd, 0.5H), 7.04 (m, 1H), 6.94 (m, 1H), 5.22 (s, 2H), 5.14 (s, 1H), 4.20 (q, 2H), 4.01 (q, 1H), 2.30 (s, 3H), 2.21 (s, 1.5H), 1.24 (t, 3H), 0.99 (t, 1.5H). MS (ESI) m/z 340.8 (M+H)⁺.

### Example 9E

### (R)-ethyl 2-acetoxy-3-(2-(benzyloxy)phenyl)propanoate

Example 9D (1.0 kg) in methanol (5.0 L) was degassed with bubbling argon for 30 minutes and was transferred to a 2 gallon Parr stainless steel reactor. The reactor was purged with argon for 30 minutes, 1,2-Bis((2R,5R)-2,5-diethylphospholano)benzene(cyclooctadiene)rhodium (I) tetrafluoroborate (17.8 g) was added, and the vessel was sealed and purged further with argon. The vessel was pressurized to 120 psi with hydrogen. The mixture was stirred under 120 psi of hydrogen with no external heating applied. After 70 hours, the reactor was vented and purged 4 times with argon. HPLC indicated complete conversion to the desired product. The mixture was transferred to a flask and concentrated. Heptane/ethyl acetate (1:1) was added, and the material turned into a cloudy mix. The flask was swirled, and a sludge crashed out. The mixture was poured through a plug of silica (1 L), eluting with 1:1 heptane/ethyl acetate. The filtrate, which contained the desired product, was concentrated to provide the title compound. ¹H NMR (400 MHz, Chloroform-d) δ ppm 7.47 (m, 2H), 7.39 (m, 2H), 7.32 (m, 1H), 7.19 (m, 2H), 6.90 (m, 2H), 5.31 (dd, 1H), 5.12 (m, 2H), 4.13 (qq, 2H), 3.35 (dd, 1H), 3.06 (dd, J=13.8, 9.2 Hz, 1H), 2.03 (s, 3H), 1.17 (t, 3H). MS (ESI) m/z 360.0 (M+NH₄)⁺.

### Example 9F

### (R)-ethyl 2-acetoxy-3-(2-hydroxyphenyl)propanoate

Example 9E (896 g) in ethanol (4.3 L) was added to wet 5% palladium on carbon catalyst (399.7 g) in a 2 gallon Parr stainless steel reactor. The reactor was purged with argon, and the mixture was stirred at 600 RPM under 50 psi of hydrogen at 25° C. for 12 hours. LC/MS indicated a single peak corresponding to desired product. The mixture was filtered through filter paper and through a 0.2 micron polypropylene membrane. The filtrate was concentrated. The crude material was transferred into a 12 L three-neck round bottom flask equipped with a mechanical stirrer and temperature probe (J-KEM controlled). The material was mixed in 5 L (about 0.5M) of heptane. The mixture was heated to about 74° C. To the hot mixture was added isopropyl acetate. The isopropyl acetate was added in 100 mL aliquots up to about 500 mL. Most of the material was dissolved. Isopropyl acetate was added in 10 mL aliquots until a clear solution formed. A total of 630 mL of isopropyl acetate was used. The mixture was heated to about 80° C. for about 10 minutes. The heat was turned off but the heating mantle was left on. Stirring was slowed to a low rate. The mixture was allowed to cool slowly overnight. The material that had formed was filtered off, washed with heptane, and dried for a few hours. The filtrate was concentrated, and the precipitation process was repeated on the residue using the same conditions to produce additional title compound. The two batches of title compound were combined. Chiral HPLC of the combined material on a Gilson HPLC system using a ChiralPak AD-H column (4.6 mm×250 mm, 3 μM) and a 5% to 50% ethanol/heptane gradient over 15 minutes indicated a single peak with a retention time of 8.9 minutes. ¹H NMR (400 MHz, DMSO-d₆) δ ppm 9.53 (s, 1H), 7.06 (m, 2H), 6.79 (m, 1H), 6.71 (td, 1H), 5.11 (dd, J=8.3, 6.0 Hz, 1H), 4.05 (q, 2H), 3.07 (dd, 1H), 2.95 (dd, 1H), 2.00 (s, 3H), 1.09 (t, 3H). MS (DCI) m/z 270.0 (M+NH₄)⁺.

### Example 9G

### (R)-ethyl 2-acetoxy-3-(5-bromo-2-hydroxyphenyl)propanoate

A dried 5 L three neck jacketed flask equipped with a mechanical stirrer and an internal temperature probe controlled by a Huber Ministat 230 chiller was charged with Example 9F (200 g) and anhydrous tetrahydrofuran (3.3 L) at room temperature under nitrogen. The mixture was cooled to −20.4° C. using the chiller. To the cooled mixture was added concentrated sulfuric acid (4.23 mL). The temperature of the reaction rose to −19.8° C. NBS (N-bromosuccinimide, 143 g) was added over a period of 10 minutes. The temperature rose from −20.3° C. to −20.0° C. during the addition. The reaction was stirred overnight at −20° C. LC/MS indicated the reaction was about 70% complete. The reaction was warmed to 0° C. with the use of the chiller and stirred 5 hours at 0° C. LC/MS indicated reaction was greater than 90% complete. The reaction was warmed to 20° C. with use of the chiller. After one hour at 20° C. LC/MS showed no sign of starting material and one major product. The reaction was cooled to 0° C. with use of the chiller. The reaction was quenched with 500 mL of water, and the temperature rose from 0° C. to about 8° C. The reaction was diluted with ethyl acetate (1.0 L), and two-phase mixture was stirred for about 20 minutes. The two-phase mixture was poured into a 6 L separatory funnel. One liter of water was added, the mixture shaken, and the layers were separated. The organic layer was washed with saturated aqueous NaHCO₃ mixture and brine. The combined aqueous layers were back-extracted one time with ethyl acetate. The combined organic extracts were dried with Na₂SO₄, filtered, and concentrated. Dichloromethane (300 mL) was added to the residue, and a slurry formed. The mixture was sonicated for 60 minutes. The material was filtered, washed with a minimum amount of dichloromethane, and dried under vacuum for an hour to produce the title compound. The material that formed in the filtrate were filtered and washed with ethyl acetate. The two batches of material were combined and dried in a vacuum oven at 50° C. for 5 hours to provide the title compound. Chiral HPLC of the material on a Gilson HPLC system using a ChiralPak AD-H column (4.6 mm×250 mm, 3 M) and a 5-50% ethanol/heptane gradient over 30 minutes indicated a single peak with a retention time of 10.6 minutes. ¹H NMR (400 MHz, DMSO-d₆) δ ppm 9.89 (s, 1H), 7.22 (m, 2H), 6.76 (dt, 1H), 5.11 (dd, 1H), 4.06 (qq, 2H), 3.05 (dd, 1H), 2.97 (dd, 1H), 2.02 (s, 3H), 1.10 (t, 3H). MS (ESI) m/z 332.8 (M+H)⁺.

### Example 9H

### (R)-ethyl 2-acetoxy-3-(5-bromo-2-((4-methoxybenzyl)oxy)phenyl)propanoate

A mixture of 4-methoxybenzyl alcohol (6.51 g), triphenylphosphine (12.36 g). Example 9G (12.0 g) and N,N,N′,N′-tetramethylazodicarboxamide (8.11 g) were dissolved in anhydrous toluene (200 mL) at 0° C. The mixture was stirred at 0° C. for 2 hours and was allowed to be warmed to room temperature overnight. The reaction mixture was directly purified by silica gel chromatography (330 g RediSep® Gold column, 10-40% ethyl acetate in hexane) to provide the title compound. MS (ESI) m/z 470 (M+NH₄)⁺.

### Example 91

### (R,E)-ethyl 2-acetoxy-3-(2-((4-methoxybenzyl)oxy)-5-(pent-1-en-1-yl)phenyl)propanoate

A mixture of Example 9H (10.12 g), (E)-pent-1-en-1-ylboronic acid (5.11 g), 2-dicyclohexylphosphino-2′,6′-dimethoxybiphenyl (1.289 g), palladium (II)acetate (0.503 g) and cesium fluoride (10.22 g) in a 500 mL round-bottom flask was purged with nitrogen. Anhydrous 1,4-dioxane (200 mL) was added under nitrogen. The mixture was purged with nitrogen again and was stirred at room temperature for 4 hours. The mixture was partitioned between ethyl acetate (400 mL) and brine (500 mL). The organic phase was washed with brine and concentrated. The residue was purified by silica gel chromatography (5-30% ethyl acetate in heptane) to provide the title compound. MS (ESI) m/z 458 (M+NH₄)⁺.

### Example 9J

### (R)-ethyl 2-acetoxy-3-(5-formyl-2-((4-methoxybenzyl)oxy)phenyl)propanoate

To a mixture of Example 91 (9.68 g) and iodobenzene diacetate (15.78 g) in a mixture of tetrahydrofuran (170 mL) and water (8.5 mL) was added 2,6-dimethylpiperidine (6.55 mL) and osmium tetroxide (0.1 M mixture in water, 4.26 mL). The reaction mixture was stirred at room temperature for 4 hours. The reaction mixture was partitioned between ethyl acetate and brine. The organic phase was washed with brine and concentrated. The residue was purified by silica gel chromatography (5-40% ethyl acetate in heptane) to provide the title compound. MS (ESI) m/z 418 (M+NH₄)⁺.

### Example 9K

### (R)-ethyl 3-(5-formyl-2-((4-methoxybenzyl)oxy)phenyl)-2-hydroxypropanoate

Example 9J (7.22 g) in anhydrous ethanol (160 mL) was treated with 21% sodium ethoxide mixture in ethanol (0.336 mL). The reaction mixture was stirred at room temperature for 5 hours and was quenched by the addition of acetic acid (0.103 mL). The volatiles were removed, and the residue was partitioned between ethyl acetate and brine. The organic phase was washed with brine and concentrated. The residue was purified by silica gel chromatography (5-50% ethyl acetate in heptane) to provide the title compound. MS (ESI) m/z 376 (M+NH₄)⁺.

### Example 9L

### (R)-ethyl 2-((5-bromo-6-(4-fluorophenyl)thieno[2,3-d]pyrimidin-4-yl)oxy)-3-(5-formyl-2-((4-methoxybenzyl)oxy)phenyl)propanoate

A mixture of Example 9K (5.28 g) and Example 1L (5.32 g) was suspended in 160 mL of anhydrous tert-butanol under nitrogen. Cesium carbonate (16.32 g) was added, and the mixture was stirred at 65° C. for 5 hours. After cooling, the reaction mixture was partitioned between ethyl acetate and brine. The organic phase was washed with brine, and concentrated. The residue was purified by silica gel chromatography (10-60% ethyl acetate in heptane) to provide the title compound. MS (ESI) m/z 666 (M+H)⁺.

### Example 9M

### 2-(4-bromo-2-chlorophenyl)-1,3-dioxane

A 3 L, three neck round bottom flask fit with a Dean-Stark trap and reflux condenser was charged with 4-bromo-2-chlorobenzaldehyde (200 g), toluene (1519 mL), propane-1,3-diol (110 mL) and p-toluenesulfonic acid monohydrate (1.1 g). The reaction was heated to reflux (112° C. internal) under Dean-Stark conditions, producing 18 mL of water in about 2 hours. The reaction mixture was cooled to room temperature and was poured into saturated aqueous sodium bicarbonate (600 mL) and ethyl acetate (500 mL). The layers were separated, and the aqueous layer was extracted with ethyl acetate (500 mL). The combined organics were dried (anhydrous MgSO₄) and treated with charcoal with stirring overnight. The mixture was filtered through a plug of diatomaceous earth and the filtrate was concentrated by rotary evaporation to provide the title compound. The crude material was placed in a vacuum oven overnight at 50° C. and was used in the next step without further purification. ¹H NMR (400 MHz, chloroform-d) δ ppm 7.57 (d, 1H), 7.51 (d, 1H), 7.42 (dd, 1H), 5.74 (s, 1H), 4.29-4.19 (m, 2H), 4.05-3.91 (m, 2H), 2.31-2.13 (m, 1H), 1.43 (dtt, 1H).

### Example 9N

### 2-(4-bromo-2-chloro-3-methylphenyl)-1,3-dioxane

A 5-neck, 5 L round bottom reactor was fit with overhead stirring, thermocouple/JKEM, addition funnels and nitrogen inlet. The assembled reactor was dried with a heat gun under nitrogen. N,N-Diisopropylamine (138 mL) and tetrahydrofuran (1759 mL) were added to the reactor under a flow of nitrogen. The clear, colorless mixture was cooled to about −76° C. (internal) upon which time n-butyllithium (369 mL, 2.5 M) was added via addition funnel, keeping the temperature below −68° C. The light yellow mixture was stirred at −76° C. for 45 minutes to generate lithium diisopropylamide (LDA). A tetrahydrofuran (500 mL) mixture of Example 9M (244.08 g) was added dropwise via addition funnel (over 45 minutes) to the LDA mixture, keeping the temperature below −68° C. The mixture was stirred for 2 hours at −76° C. Iodomethane (57.7 mL) was added dropwise over 1 hour via addition funnel (very exothermic), and the temperature was kept below −70° C. during the addition. The reaction mixture was allowed to warm slowly to room temperature and was stirred overnight. In the morning, water and saturated aqueous ammonium chloride were added along with ethyl acetate (1 L). The layers were separated by pump, and the aqueous layer was extracted with ethyl acetate (twice) pumping the top layer into a separatory funnel. The combined organics were dried (anhydrous MgSO₄), filtered through diatomaceous earth and concentrated by rotary evaporation to provide crude desired product. The material (246 g) was slurried in 550 mL isopropyl alcohol. The mixture was heated to about 80° C. With stirring, the mixture was allowed to cool slowly to room temperature. Copious amounts of material formed, and the flask was placed in the freezer (−16° C.). After 1 hour, the material was broken up and 400 mL of ice cold isopropyl alcohol was added. The mixture was slurried and filtered through paper, washing quickly with cold isopropyl alcohol. The material was allowed to dry on the filter bed and was placed in the vacuum oven for 5 hours (50° C.) to provide the title compound. ¹H NMR (400 MHz, Chloroform-d) δ ppm 7.50 (d, 1H), 7.41 (d, 1H), 5.77 (s, 1H), 4.25 (ddd, 2H), 4.01 (td, 2H), 2.53 (s, 3H), 2.34-2.13 (m, 1H), 1.44 (ddt, 1H). MS (ESI) m=308.0 (M+NH₄)⁺.

### Example 90

### 2-(3-chloro-4-(1,3-dioxan-2-yl)-2-methylphenyl)-4,4,5,5-tetramethyl-1,3,2-dioxaborolane

A 3-neck, 5 L round bottom flask fitted with a thermocouple/JKEM, dry ice acetone bath, overhead stirring, nitrogen inlet and outlets and addition funnel was charged with Example 9N (100 g) and tetrahydrofuran (1715 mL) under a positive flow of nitrogen. The mixture was cooled to −76° C. (internal) and n-butyllithium (151 mL, 2.5 M) was added dropwise via addition funnel, observing a temperature increase of 5-8° C. The mixture remained clear and colorless and was stirred for 10 minutes at −76° C., 2-Isopropoxy-4,4,5,5-tetramethyl-1,3,2-dioxaborolane (84 mL) was added dropwise (exothermic) at a rate to keep the temperature below −68° C. The reaction mixture was stirred at −76° C. for about 30 minutes, warmed to room temperature, and stirred for 3 hours. The reaction mixture was concentrated by rotary evaporation. The water bath was set to 80° C. and the evaporator was switched to high vacuum for 1 hour. Water and ethyl acetate were added to the residue, and the layers were separated. The water layer was extracted with ethyl acetate, and the combined organics were dried (anhydrous MgSO₄), filtered and concentrated. The crude material was triturated with ice-cold methanol, filtered through paper, and dried on the filter bed and vacuum oven (50° C.) to provide the title compound. ¹H NMR (400 MHz, DMSO-d₆) δ ppm 7.59 (d, 1H), 7.45 (d, 1H), 5.76 (s, 1H), 4.14 (ddd, 2H), 3.96 (td, 2H), 2.53 (s, 2H), 2.09-1.94 (m, 1H), 1.50-1.39 (m, 1H), 1.31 (s, 9H). MS (ESI) m/z 339.3 (M+H)⁺.

### Example 9P

### (2R)-ethyl 2-((5-((1S)-3-chloro-4-(1,3-dioxan-2-yl)-2-methylphenyl)-6-(4-fluorophenyl)thieno[2,3-d]pyrimidin-4-yl)oxy)-3-(5-formyl-2-((4-methoxybenzyl)oxy)phenyl)propanoate

A 250 mL round-bottom flask was charged with Example 9L (9.32 g). Example 90 (6.16 g), potassium phosphate (8.92 g), and bis(di-tert-butyl (4-dimethylaminophenyl)phosphine)dichloropalladium (II) (992 mg). The flask was purged with nitrogen, after which tetrahydrofuran (100 mL) and water (25 mL) were added. The reaction mixture was purged with nitrogen again and was stirred at room temperature overnight. The reaction mixture was partitioned between ethyl acetate and brine. The organic phase was washed with brine, and concentrated. The residue was purified by silica gel chromatography (10-60% ethyl acetate in heptane) to provide the title compound. MS (ESI) m/z 797 (M+H)⁺.

### Example 9Q

### ethyl (7R,20S)-18-chloro-1-(4-fluorophenyl)-10-[(4-methoxyphenyl)methoxy]-19-methyl-15-[2-(4-methylpiperazin-1-ylethyl]-7,8,15,16-tetrahydro-14H-17,20-etheno-13,9-(metheno)-6-oxa-2-thia-3,5,15-triazacyclooctadeca[1,2,3-cd]indene-7-carboxylate

To Example 9P (8.8 g) in a mixture of anhydrous dichloromethane (100 mL) and acetic acid (20 mL) was added 2-(4-methylpiperazin-1-yl) ethanamine (3.16 g). The mixture was stirred at room temperature for 1 hour before sodium triacetoxyborohydride (7.02 g) was added. The reaction mixture was stirred at room temperature overnight. The volatiles were removed by rotary evaporation, and the residue was dissolved in tetrahydrofuran (45 mL) and water (7.5 mL). The mixture was cooled to 0° C. and trifluoracetic acid (45 mL) was added. After the addition, the cooling bath was removed, and the mixture was stirred at room temperature for 4 hours. The mixture was diluted with ethyl acetate. The mixture was washed with a pre-cooled diluted sodium hydroxide mixture (contained about 60 mL of 50% sodium hydroxide mixture, pH 10) and brine. The organic phase was concentrated. The intermediate was dissolved in anhydrous dichloromethane (100 mL). Anhydrous magnesium sulfate (25 g) was added. The mixture was stirred at room temperature overnight, and sodium triacetoxyborohydride (7.02 g) was added. The reaction mixture was stirred at room temperature for 4 hours. The material was filtered off, and the filtrate was directly purified by silica gel chromatography (0-20% methanol containing 3% ammonium hydroxide in dichloromethane) to provide the title compound. MS (ESI) m/z 850 (M+H)⁺.

### Example 9R

### ethyl (7R,20S)-18-chloro-1-(4-fluorophenyl)-10-hydroxy-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

Example 9Q (2.9 g) was dissolved in anhydrous trifluoracetic acid (60 mL), and the mixture was heated at 45° C. for 1 hour. Anhydrous toluene (60 mL) was added, and the mixture was concentrated. Anhydrous toluene (60 mL) was added to the residue. The mixture was concentrated and dried under vacuum for 2 hours. Anhydrous ethanol (100 mL) was added, and the mixture was stirred at room temperature over a weekend. The volatiles were removed, and the residue was treated with triethylamine (2.5 mL) and loaded onto a silica gel column that was eluted with 0-20% methanol containing 3% ammonium hydroxide in dichloromethane to provide the title compound. MS (ESI) m/z 731 (M+H)⁺.

### Example 9S

### methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-glucopyranoside ethyl ester

A mixture of Example 9C (0.018 g), Example 9R (0.023 g) and cesium carbonate (0.020 g) were stirred together in dimethylformamide (0.50 mL). The reaction mixture was stirred overnight and was diluted with a mixture of N,N-dimethylformamide (1.5 mL), water (0.5 mL) and 2,2,2-trifluoroacetic acid (5 ML). The mixture was purified by Prep HPLC using a Gilson 2020 system (Luna™ column, 250×50 mm, flow 70 mL/minute) using a gradient of 5-100% acetonitrile in water (0.1% TFA) over 30 minutes. The desired product-containing fractions were lyophilized to provide the title compound. MS (APCI) m/z 1216.5 (M+H)⁺.

### Example 9T

### methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-glucopyranoside

To Example 9S (0.005 g) in a mixture of tetrahydrofuran (0.100 mL) and methanol (0.100 mL) was added lithium hydroxide hydrate (3.15 mg) in water (0.100 mL). The resulting mixture was stirred at room temperature for 3 days and was diluted with a mixture of N,N-dimethylformamide (0.5 mL), water (0.5 mL) and 2,2,2-trifluoroacetic acid (6 μL). The mixture was purified by Prep HPLC using a Gilson 2020 system (Luna™ column, 250×30 mm, flow 40 mL/minutes) using a gradient of 10-65% acetonitrile in water (0.1% TFA) over 35 minutes. The desired product fractions were lyophilized to provide the title compound. ¹H NMR (400 MHz, DMSO-d₆) δ ppm 8.61-8.54 (m, 1H), 8.29-8.21 (m, 1H), 7.43 (d, 1H), 7.24 (d, 1H), 7.22-7.16 (m, 1H), 7.15-7.07 (m, 2H), 7.06-6.99 (m, 1H), 6.78 (d, 1H), 6.46 (d, 1H), 5.89 (dd, 1H), 5.17 (d, 1H), 5.03 (d, 1H), 4.55 (d, 1H), 4.32-4.24 (m, 1H), 4.17 (s, 1H), 4.11 (dd, 1H), 4.02 (s, 1H), 3.74-3.62 (m, 2H), 3.26-2.90 (m, 31H), 2.73 (s, 3H), 1.69 (s, 3H). MS (ESI) m/z 1062.4 (M+H)⁺.

### Example 10

### methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-mannopyranoside

### Example 10A

### (2S,3S,4S,5R,6R)-2-methoxy-6-((trityloxy)methyl)tetrahydro-2H-pyran-3,4,5-triyl triacetate

To a mixture of (2R,3S,4S,5S,6S)-2-(hydroxymethyl)-6-methoxytetrahydro-2H-pyran-3,4,5-triol (2.0 g) in pyridine (35 mL) was added triphenylmethyl chloride (3.16 g) and N,N-dimethylpyridin-4-amine (0.315 g). The reaction mixture was stirred overnight at room temperature, and heated to 80° C. for 4 hours. The reaction mixture was cooled to room temperature and acetic anhydride (5.83 mL) was added. Stirring was continued at room temperature for 4 hours. The reaction mixture was poured into water (200 mL) and extracted three times with ethyl acetate. The combined extracts were washed with brine and concentrated. The crude material was purified by silica gel chromatography, using 2-50% ethyl acetate in heptanes as the eluent, to provide the title compound. ¹H NMR (400 MHz, CDCl₃) δ ppm 7.49-7.43 (m, 6H), 7.33-7.19 (m, 9H), 5.35-5.19 (m, 3H), 4.76 (d, 1H), 3.89 (dt, 1H), 3.47 (s, 3H), 3.20 (d, 2H), 2.17 (s, 3H), 1.96 (s, 3H), 1.73 (s, 3H). MS (ESI) m/z 585.2 (M+Na)⁺.

### Example 10B

### (2R,3R,4S,5S,6S)-2-(hydroxymethyl)-6-methoxytetrahydro-2H-pyran-3,4,5-triyl triacetate

Example 10A (4.14 g) in acetic acid (50 mL) was heated to 80° C. and water (25 mL) was added to the reaction. The reaction mixture was stirred for 1 hour at 85° C., cooled to room temperature, poured into water (50 mL), and extracted with dichloromethane (75 mL). The organic layer was washed with brine (50 mL), dried over magnesium sulfate, filtered, and concentrated. The residue was loaded onto silica gel (Teledyne Isco RediSep® Rf gold 120 g) and was eluted using a gradient of 5-75% heptanes/ethyl acetate. The desired product containing fractions were combined and concentrated. The residue was dissolved in minimal dichloromethane, and was diluted with diethyl ether and concentrated to provide the title compound. ¹H NMR (400 MHz, CDCl₃) δ ppm 5.40 (dd, 1H), 5.29-5.19 (m, 2H), 4.73 (d, 1H), 3.79-3.68 (m, 2H), 3.67-3.60 (m, 1H), 3.41 (s, 3H), 2.37 (dd, 1H), 2.15 (s, 3H), 2.08 (s, 3H), 2.01 (s, 3H). MS (ESI) m/z 338.0 (M+NH₄)⁺.

### Example 10C

### (2S,3S,4S,5R,6R)-2-methoxy-6-((4-(4,4,5,5-tetramethyl-1,3,2-dioxaborolan-2-yl)phenoxy)methyl)tetrahydro-2H-pyran-3,4,5-triyl triacetate

To a mixture of 4-(4,4,5,5-tetramethyl-1,3,2-dioxaborolan-2-yl)phenol (0.760 g). Example 10B (1.66 g) and triphenylphosphine (1.359 g) in toluene (20 mL) was added di-tert-butyl azodicarboxylate (1.193 g) and the reaction was heated to 50° C. for 3 hours. The reaction mixture was concentrated to ˜½ volume and loaded onto silica gel (Teledyne Isco RediSep® Rf gold 120 g). The column was eluted using a gradient of 5-75% heptanes/ethyl acetate. The desired product containing fractions were combined, taken up in diethyl ether and concentrated to provide the title compound. ¹H NMR (400 MHz, CDCl₃) δ ppm 7.81-7.68 (m, 2H), 6.95-6.83 (m, 2H), 5.42-5.32 (m, 2H), 5.28-5.24 (m, 1H), 4.73 (d, 1H), 4.18-4.06 (m, 3H), 3.43 (s, 3H), 2.16 (s, 3H), 2.02 (s, 3H), 2.01 (s, 3H), 1.33 (s, 12H). MS (ESI) m/z 539.8 (M+NH₄)⁺.

### Example 10D

### (2R,3R,4S,5S,6S)-2-((4-(4-(hydroxymethyl)pyrimidin-2-yl)phenoxy)methyl)-6-methoxytetrahydro-2H-pyran-3,4,5-triyl triacetate

A mixture of (2-chloropyrimidin-4-yl)methanol (100 mg), Example 10C (470 mg) and tetrakis(triphenylphosphine)palladium (0) (80 mg) in tetrahydrofuran (4.4 mL) and saturated aqueous sodium bicarbonate mixture (2.5 mL) was heated to 75° C. under an atmosphere of nitrogen for 4 hours. The reaction mixture was cooled, diluted with ethyl acetate (50 mL) and washed with water (25 mL) and brine (25 mL). The organic layer was dried over magnesium sulfate, filtered, and concentrated. The residue was loaded onto silica gel (Teledyne Isco RediSep® Rf gold 80 g) and was eluted using a gradient of 5-85% heptanes/ethyl acetate. The desired product containing fractions were combined to provide the title compound. ¹H NMR (400 MHz, CDCl₃) δ ppm 8.70 (d, 1H), 8.45-8.36 (m, 2H), 7.10 (d, 1H), 7.05-6.96 (m, 2H), 5.44-5.36 (m, 2H), 5.34-5.23 (m, 1H), 4.79 (d, 2H), 4.76 (d, 1H), 4.17 (d, 3H), 3.63 (t, 1H), 3.45 (s, 3H), 2.17 (s, 3H), 2.05 (s, 3H), 2.01 (s, 3H). MS (ESI) m/z 505.3 (M+H)⁺.

### Example 10E

### (2R,3R,4S,5S,6S)-2-((4-(4-(chloromethyl)pyrimidin-2-yl)phenoxy)methyl)-6-methoxytetrahydro-2H-pyran-3,4,5-triyl triacetate

To a mixture of Example 10D (0.230 g) in dichloromethane (5 mL) was added triphenylphosphine (0.155 g) followed by N-chlorosuccinimide (0.067 g.) and the reaction was stirred at 0° C. for 3 hours. The reaction mixture was loaded onto silica gel (Teledyne Isco RediSep® Rf gold 40 g) and was eluted using a gradient of 5-75% heptanes/ethyl acetate. The desired product containing fractions were combined to provide the title compound. ¹H NMR (400 MHz, CDCl₃) δ ppm 8.79 (d, 1H), 8.45-8.35 (m, 2H), 7.35 (d, 1H), 7.05-6.94 (m, 2H), 5.45-5.34 (m, 2H), 5.31-5.23 (m, 1H), 4.75 (d, 1H), 4.65 (s, 2H), 4.23-4.10 (m, 3H), 3.45 (s, 3H), 2.17 (s, 3H), 2.05 (s, 3H), 2.01 (s, 3H). MS (ESI) m/z 523.1 (M+H)⁺.

### Example 10F

### methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-mannopyranoside ethyl ester

To Example 10E (0.043 g) and Example 9R (0.040 g) in dimethylformamide (0.30 mL) was added cesium carbonate (0.054 g) and the reaction mixture was stirred at room temperature. After stirring for 5 hours, the reaction was diluted with a mixture of N,N-dimethylformamide (1.5 mL), water (0.5 mL) and 2,2,2-trifluoroacetic acid (0.013 mL). The mixture was purified by prep HPLC using a Gilson 2020 system (Luna™ column, 250×50 mm, flow 70 mL/minutes) using a gradient of 5-85% acetonitrile/water (0.1% TFA) over 30 minutes. The desired product containing fractions were lyophilized to provide the title compound. MS (APCI) m/z 1216.5 (M+H)⁺.

### Example 10G

### methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-a-D-mannopyranoside

To Example 10F (0.024 g) in a mixture of tetrahydrofuran (0.150 mL) and methanol (0.150 mL) was added lithium hydroxide hydrate (0.015 g) in water (0.100 mL) and the resulting mixture was stirred at room temperature. After stirring for 3 days, the reaction mixture was diluted with a mixture of N,N-dimethylformamide (0.5 mL), water (0.5 mL) and 2,2,2-trifluoroacetic acid (0.035 mL). The mixture was purified by prep HPLC using a Gilson 2020 system (Luna™ column, 250×50 mm, flow 70 mL/minutes) using a gradient of 5-60% acetonitrile in water over 30 minutes. The desired product containing fractions were lyophilized to provide the title compound. ¹H NMR (400 MHz, DMSO-d₆) δ ppm 8.60 (d, 1H), 8.57 (s, 1H), 8.31-8.20 (m, 2H), 7.46 (d, 1H), 7.26 (d, 1H), 7.23-7.07 (m, 6H), 7.07-7.00 (m, 2H), 6.81 (d, 1H), 6.46 (d, 1H), 5.91 (dd, 2H), 5.23-5.00 (m, 4H), 4.51 (d, 1H), 4.36-4.22 (m, 3H), 4.11 (dt, 4H), 3.67-3.51 (m, 11H), 3.23 (d, 3H), 3.21-3.07 (m, 6H), 3.00 (s, 4H), 2.75 (s, 3H). MS (ESI) m/z 1062.1 (M+H)⁺.

### Example 11

### methyl 6-O-{4-[4-({[(7R,20S)-7-carboxy-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-2,3,4-tri-O-methyl-a-D-mannopyranoside

### Example 11A

### 4,4,5,5-tetramethyl-2-(4-(((2R,3R,4S,5S,6S)-3,4,5,6-tetramethoxytetrahydro-2H-pyran-2-yl)methoxy)phenyl)-1,3,2-dioxaborolane

The title compound was prepared by substituting Example 6C for (2R,3R,4S,5R,6S)-2-(hydroxymethyl)-6-methoxytetrahydro-2H-pyran-3,4,5-triyl triacetate in Example 9A. MS (DCI) m/z 456.2 (M+NH₄)⁺.

### Example 11B

### (2-(4-(((2R,3R,4S,5S,6S)-3,4,5,6-tetramethoxytetrahydro-2H-pyran-2-yl)methoxy)phenyl)pyrimidin-4-yl)methanol

The title compound was prepared by substituting Example 11A for Example 9A in Example 9B. MS (DCI) m/z 421.1 (M+H)⁺.

### Example 11C

### 4-(chloromethyl)-2-(4-(((2R,3R,4S,5S,6S)-3,4,5,6-tetramethoxytetrahydro-2H-pyran-2-yl)methoxy)phenyl)pyrimidine

The title compound was prepared by substituting Example 11B for Example 9B in Example 9C. MS (DCI) m/z 439.0 (M+H)⁺.

### Example 11D

### methyl 6-O-{4-[4-({[(7R)-18-chloro-7-(ethoxycarbonyl)-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-2,3,4-tri-O-methyl-a-D-mannopyranoside

The title compound was prepared by substituting Example 11C for Example 9C in Example 9T. MS (ESI) m/z 1132.4 (M+H)⁺.

### Example 11E

### methyl 6-O-{4-[4-({[(7R,20S)-7-carboxy-18-chloro-1-(4-fluorophenyl)-19-methyl-15-[2-(4-methylpiperazin-1-ylethyl]-7,8,15,16-tetrahydro-14H-17,20-etheno-13,9-(metheno)-6-oxa-2-thia-3,5,15-triazacyclooctadeca[1,2,3-cd]inden-10-yl]oxy}methyl)pyrimidin-2-yl]phenyl}-2,3,4-tri-O-methyl-a-D-mannopyranoside

The title compound was prepared by substituting Example 11D for Example 9T in Example 9U. ¹H NMR (500 MHz, dimethylsulfoxide-d₆) δ ppm 8.64 (d, 1H), 8.60 (s, 1H), 8.29 (d, 2H), 7.51 (d, 1H), 7.29 (d, 1H), 7.23 (m, 3H), 7.14 (m, 3H), 7.09 (d, 2H), 6.85 (d, 1H), 6.51 (s, 1H), 5.94 (m, 1H), 5.22 (d, 1H), 5.08 (d, 1H), 4.78 (d, 1H), 4.32 (br m, 2H), 4.20 (m, 4H), 3.67 (m, 2H), 3.60 (m, 2H), 3.41 (m, 8H), 3.40 (s, 3H), 3.38 (s, 3H), 3.35 (s, 3H), 3.30 (s, 3H), 3.22 (m, 2H), 3.17 (m, 2H), 3.06 (m, 2H), 2.80 (s, 3H), 1.74 (s, 3H). MS (ESI) m/z 1104.5 (M+H)⁺.

### Example 12

### methyl 6-O-{4-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-a-D-mannopyranoside

### Example 12A

### tert-butyl 2-acetoxy-2-(diethoxyphosphoryl)acetate

A 3 L jacketed round bottom flask, equipped with an overhead stirrer, was charged with glyoxylic acid monohydrate (15 g) and diethyl phosphite (20.82 mL) and was heated to a 60° C. jacket temperature with stirring. The flask headspace was continuously purged with a nitrogen sweep. After stirring overnight, dichloromethane (250 mL) was added, the reaction was cooled to an internal temperature of 5° C. Pyridine (13.05 mL) was added dropwise. After stirring for 1 hour at the same temperature, acetyl chloride (11.47 mL) was added dropwise over 20 minutes. The reaction mixture was warmed to 20° C. stirred for 1.5 hours, and cooled to 5° C. internal temperature. Pyridine (19.57 mL) was added slowly, tert-Butanol (15.43 mL) was added in one portion followed by dropwise addition of 2,4,6-tripropyl-1,3,5,2,4,6-trioxatriphosphinane 2,4,6-trioxide (144 mL, 50% by weight in ethyl acetate) over 20 minutes. After stirring for 1 hour, the reaction was warmed to 20° C. and was stirred overnight. The reaction mixture was cooled to 5° C. and 1 N aqueous hydrochloric acid (200 mL) was added slowly. The biphasic mixture was stirred for 30 minutes at 20° C., and poured into a separatory funnel. Dichloromethane (400 mL) and 1 N aqueous hydrochloric acid (250 mL) were added and the mixture was separated. The aqueous layer was extracted with dichloromethane (400 mL), and the combined organic layers were washed with a mixture of water (300 mL) and saturated aqueous sodium chloride solution (300 mL), and dried over anhydrous magnesium sulfate, filtered and concentrated under reduced pressure. The crude material was purified by plug filtration on silica gel eluting with 1:1 ethyl acetate/heptanes to give the title compound after concentration under reduced pressure. ¹H NMR (400 MHz, Chloroform-d) δ ppm 5.32 (d, 1H), 4.29-4.18 (m, 4H), 2.21 (s, 3H), 1.37 (tdd, 6H). MS (ESI) m/z 255.0 (M-tert-butyl+2H)⁺.

### Example 12B

### (E)-tert-butyl 2-acetoxy-3-(2-(benzyloxy)-5-((tert-butyldimethylsilyl)oxy)phenyl)acrylate

An oven dried 2 L 3-neck round bottomed flask equipped with overhead stirring was charged with anhydrous lithium chloride (5.55 g). The flask was purged with a sweep of argon for 10 minutes and anhydrous tetrahydrofuran (350 mL) was added. A mixture of Example 12A (40.6 g) in tetrahydrofuran (50 mL) was added. A mixture of 1,8-diazabicyclo[5.4.0]undec-7-ene (19.72 mL) in tetrahydrofuran (50 mL) was added dropwise. The stirring mixture became cloudy and was cooled in an ice-water bath to an internal temperature of 15° C. A mixture of Example 1A (32 g) in tetrahydrofuran (50 mL) was added over 30 minutes. The reaction mixture was stirred overnight, cooled to an internal temperature of 5° C. and quenched by addition of 1% by weight aqueous citric acid (700 mL). Ethyl acetate (400 mL) was added and the layers were separated. The combined organic layers were washed with saturated aqueous sodium chloride solution (400 mL), and dried over anhydrous magnesium sulfate, filtered and concentrated under reduced pressure. The crude material was purified by flash column chromatography on a Grace Reveleris system using a Teledyne Isco RediSep® Gold 330 g column, eluting with a 0-25% ethyl acetate/heptanes gradient to give the title compound as a 9:1 mixture of E- and Z-isomers. E-isomer ¹H NMR (501 MHz, Chloroform-d) δ ppm 7.39 (ddt, 2H), 7.36 (ddd, 2H), 7.32-7.27 (m, 1H), 6.88 (dd, 1H), 6.85 (d, 1H), 6.76 (d, 1H), 6.71 (ddd, 1H), 5.01 (s, 2H), 2.22 (s, 3H), 1.34 (s, 9H), 0.97 (s, 9H), 0.17 (s, 6H). MS (ESI) m/z 515.9 (M+NH₄)⁺. This isomer was assigned E by 2D NOE experiments. Z-isomer: ¹H NMR (501 MHz, Chloroform-d) δ ppm 7.74 (s, 1H), 7.45 (ddt, 2H), 7.38 (ddd, 2H), 7.35-7.30 (m, 1H), 7.29-7.26 (m, 1H), 6.83 (d, 1H), 6.79 (dd, 1H), 5.06 (s, 2H), 2.30 (d, 3H), 1.53 (s, 9H), 0.99 (s, 9H), 0.18 (s, 6H). MS (ESI) m/z 515.9 (M+NH₄)⁺. This isomer was assigned Z by 2D NMR experiments.

### Example 12C

### (R)-tert-butyl 2-acetoxy-3-(2-(benzyloxy)-5-((tert-butyldimethylsilyl)oxy)phenyl)propanoate

A 600 mL stainless steel reactor was charged with (1,2-bis[(2R,5R)-2,5-diethylphospholano]benzene(1,5-cyclooctadiene)rhodium (I) trifluoromethanesulfonate (1.88 g), followed by a solution of Example 12B (34.86 g) in methanol (350 mL). The reactor was purged with nitrogen 3 times and 2 times with hydrogen. The mixture was stirred at 1200 RPM under 120 psi of hydrogen with no external heating for 24 hours. The solution was concentrated under reduced pressure, suspended in 5:1 heptanes/dichloromethane (70 mL), and filtered through a pad of diatomaceous earth. The filtrate was concentrated under reduced pressure and purified on a Grace Reveleris system using a 750 g Teledyne Isco Redisep® gold column eluting with an ethyl acetate/heptanes gradient (0-25%). The desired fractions were concentrated under reduced pressure to provide the title compound. ¹H NMR (400 MHz, Chloroform-d) δ ppm 7.45 (d, 2H), 7.42-7.34 (m, 2H), 7.34-7.28 (m, 1H), 6.77 (d, 1H), 6.70 (d, 1H), 6.67 (dd, 1H), 5.19 (dd, 1H), 5.05 (d, 1H), 5.01 (d, 1H), 3.29 (dd, 1H), 2.92 (dd, 1H), 2.03 (s, 3H), 1.40 (s, 9H), 0.97 (s, 9H), 0.16 (s, 6H). MS (DCI) m/z 518.2 (M+NH₄)⁺.

### Example 12D

### (R)-tert-butyl 3-(2-(benzyloxy)-5-((tert-butyldimethylsilyl)oxy)phenyl)-2-hydroxypropanoate

An oven dried 250 mL 3-neck flask was charged with Example 12C (27.46 g). The flask was equipped with a magnetic stir bar, rubber septa, and vacuum purged with nitrogen gas twice. Anhydrous ethanol (274 mL) was added as the mixture was stirred. To the stirring solution was added dropwise sodium ethoxide (21% wt in ethanol, 1.024 mL). The reaction mixture was stirred for three hours at ambient temperature and quenched by addition of acetic acid (0.3 mL). Most of the solvents were removed by rotary evaporation, and the material was diluted with ethyl acetate (300 mL), Saturated aqueous sodium bicarbonate was added (300 mL). The layers were separated and the aqueous layer was extracted with ethyl acetate (300 mL). The combined organic layers were washed with saturated aqueous sodium chloride, dried over MgSO₄, treated with activated charcoal (0.5 g), and stirred for 1 hour before filtering through diatomaceous earth to provide the title compound after concentration under reduced pressure. ¹H NMR (400 MHz, Chloroform-d) δ ppm 7.48-7.42 (m, 2H), 7.42-7.36 (m, 2H), 7.36-7.29 (m, 1H), 6.79 (d, 1H), 6.75 (d, 1H), 6.67 (dd, 1H), 5.10-4.99 (m, 2fH), 4.39 (ddd, 1H), 3.16 (dd, 1H), 2.91 (d, 1H), 2.86 (dd, 1H), 1.41 (s, 9H), 0.99 (s, 9H), 0.18 (s, 6H). MS (DCI) m/z 476.2 (M+NH₄)⁺.

### Example 12E

### (R)-tert-butyl 3-(2-(benzyloxy)-5-((tert-butyldimethylsilyl)oxy)phenyl)-2-((5-bromo-6-(4-fluorophenyl)thieno[2,3-d]pyrimidin-4-yl)oxy)propanoate

A 1000 mL flask containing Example 12D (24.03 g) and Example 1L (19.08 g) was equipped with a stir bar and thermocouple for internal temperature monitoring and was sealed with a rubber septum. The flask was flushed with argon, and warm tert-butanol (262 mL) was added via cannula. Cesium carbonate (51.2 g) was added in one portion. The reaction mixture was heated to an internal temperature of 65° C. After four hours, the reaction mixture was allowed to cool to ambient temperature, diluted with methyl tert-butyl ether (100 mL) and filtered through a pad of diatomaceous earth. The filter pad was washed with ethyl acetate (2×100 mL). The solvents were evaporated and the crude material was re-dissolved in ethyl acetate (500 mL). The mixture was washed with water (300 mL) and saturated aqueous sodium chloride solution (300 mL). The organic layer was dried over anhydrous magnesium sulfate, filtered, and concentrated. The crude residue was purified on a Grace Reveleris instrument using a Teledyne Isco Redisep® Gold 750 g column, eluting with a 0-30% ethyl acetate/heptanes gradient. The desired fractions were combined and concentrated to provide the title compound. ¹H NMR (501 MHz, Chloroform-d) δ ppm 8.49 (s, 1H), 7.68-7.59 (m, 2H), 7.48-7.44 (m, 2H), 7.39-7.32 (m, 2H), 7.32-7.27 (m, 1H), 7.21-7.13 (m, 2H), 6.91 (d 1H), 6.77 (d, 1H), 6.65 (dd, 1H), 5.76 (dd, 1H), 5.07 (d, 1H), 5.04 (d, 1H), 3.49 (dd, 1H), 3.26 (dd, 1H), 1.40 (s, 9H), 0.93 (s, 9H), 0.11 (s, 3H), 0.10 (s, 3H). MS (ESI) m/z 765.2 (M+H)⁺.

### Example 12F

### (3-chloro-4-hydroxy-2-methylphenyl)boronic Acid

A 5 L 3 neck jacketed flask equipped with overhead stirring and thermocouple for internal temperature monitoring was charged with Example 1R (50 g), chloro[(tri-tert-butylphosphine)-2-(2-aminobiphenyl)]palladium (II) (5.78 g), tetrahydroxydiboron (60.7 g), and potassium acetate (55.4 g) which had been dried overnight under vacuum at 50° C. The flask was flow purged with a N₂ sweep for 2 hours, and cooled until the internal temperature of the material reached −6° C. An oven dried 2 L round bottomed flask was charged with anhydrous methanol (1129 mL) and anhydrous ethylene glycol (376 mL). The mixture was degassed by subsurface sparging with nitrogen gas for two hours and was cooled to −8° C. in an ice/ethanol bath. The solvent mixture was then transferred to the reaction flask via cannula over 10 minutes. The reaction mixture was stirred at −7° C. for 2.5 hours, and quenched by addition of water (1000 mL). The reaction mixture was allowed to stir at 0° C. for 1 hour. The mixture was filtered through a large pad of diatomaceous earth and the filter pad was washed with 1:1 water/methanol (2×500 mL). The filtrate was concentrated on a rotary evaporator until approximately 1.5 L of solvent had been removed. The mixture was extracted with ethyl acetate (2×1 L). The combined organic extracts were washed with brine, dried over anhydrous magnesium sulfate, filtered, and concentrated under reduced pressure. The crude material was treated with dichloromethane (200 mL) and filtered to provide the title compound. ¹H NMR (400 MHz, dimethylsulfoxide-d₆/deuterium oxide) δ ppm 7.19 (d, 1H), 6.75 (d, 1H), 2.38 (s, 3H). MS (ESI) m/z 412.9 (M−H)⁻.

### Example 12G

### (R)-tert-butyl 3-(2-(benzyloxy)-5-((tert-butyldimethylsilyl)oxy)phenyl)-2-(((S)-5-(3-chloro-4-hydroxy-2-methylphenyl)-6-(4-fluorophenyl)thieno[2,3-d]pyrimidin-4-yl)oxy)propanoate

A 1 L 3 neck flask equipped with overhead stirring was charged with Example 12E (30.2 g), 4-(di-tert-butylphosphino)-N,N-dimethylaniline (1.15 g). (tris(dibenzylideneacetone) dipalladium (0)) (1.806 g), and Example 12F (14.70 g). The flask was sealed with rubber septa and was flushed with argon for 15 minutes. A separate 500 mL round bottomed flask equipped with a magnetic stir bar was charged with cesium carbonate (25.7 g) and was sealed with a septum. The flask was flushed with argon for 10 minutes, and water (46.9 mL) and 1,4-dioxane (235 mL) were added. The flask was degassed by subsurface sparging with stirring for 30 minutes and the contents were transferred to the reaction flask via cannula. The reaction mixture was stirred for 60 hours and was quenched by addition of ammonium pyrrolidine-1-carbodithioate (1.296 g). The reaction mixture was stirred for 1 hour at which point ethyl acetate (200 mL) and water (100 mL) were added. The biphasic mixture was filtered through a pad of diatomaceous earth, washing with ethyl acetate (100 mL) and water (50 mL). The layers were separated and the aqueous layer was extracted with ethyl acetate (200 mL). The combined organic layers were washed with a solution of saturated aqueous sodium chloride, dried over anhydrous magnesium sulfate, filtered and concentrated under reduced pressure. The crude material was purified by flash column chromatography using a Grace Reveleris system using a Teledyne Isco Redisep® Gold 750 g column eluting with a 0-30% ethyl acetate/heptanes gradient. The desired fractions were collected and concentrated under reduced pressure to give the title compound. ¹H NMR (501 MHz, dimethylsulfoxide-d₆) δ ppm 10.10 (s, 1H), 8.61 (s, 1H), 7.43-7.38 (m, 2H), 7.36-7.24 (m, 5H), 7.24-7.18 (m, 2H), 6.92 (d, 1H), 6.89 (d, 1H), 6.80 (d, Hz, 1H), 6.68 (dd, 1H), 6.43 (d, 1H), 5.34 (t, 1H), 5.03 (s, 2H), 2.70-2.60 (m, 2H), 1.91 (s, 3H), 1.17 (s, 9H), 0.89 (s, 9H), 0.09 (s, 3H), 0.08 (s, 3H). MS (ESI) m/z 827.1 (M+H)⁺.

### Example 12H

### (R)-3-(allyloxy)propane-1,2-diol

To a 250 mL round bottom containing (S)-4-((allyloxy)methyl)-2,2-dimethyl-1,3-dioxolane (7.08 g) was added methanol (100 mL) and p-toluenesulfonic acid monohydrate (0.782 g). The mixture was heated to 50° C. for 18 hours, and at 60° C. for 4 hours. The mixture was cooled to room temperature, and potassium carbonate (1.704 g) and 5 g MgSO₄ were added. The material was filtered and washed with ethyl acetate. The mixture was concentrated, and the residue was chromatographed on silica gel using 20-80% ethyl acetate in heptanes as the eluent, to provide the title compound. ¹H NMR (400 MHz, dimethyl sulfoxide-d₆) δ ppm 5.87 (tdd, 1H), 5.25 (dd, 1H), 5.13 (dd, 1H), 4.62 (d, 1H), 4.46 (t, 1H), 3.94 (ddd, 2H), 3.58 (m, 1H), 3.39 (m, 1H), 3.30 (m, 3H).

### Example 12I

### (S)-3-(allyloxy)-2-hydroxypropyl 4-methylbenzenesulfonate

A 1 L 3 necked round bottomed flask equipped with a magnetic stir bar was charged with a solution of Example 12H (45.8 g) in dichloromethane (500 mL), 4-Dimethylaminopyridine (0.572 g) and N-ethyl-N-isopropylpropan-2-amine (60.3 mL) were then added sequentially. Solid 4-methylbenzene-1-sulfonyl chloride (33 g) was added portion wise and the reaction was heated to an internal temperature of 40° C. overnight. Upon cooling to ambient temperature, saturated aqueous ammonium chloride was added (300 mL). The layers were separated, and the organic layer was washed with saturated sodium chloride (200 mL), dried over anhydrous magnesium sulfate, filtered and concentrated under reduced pressure. The crude material was purified by flash column chromatography on a Grace Reveleris System using a Teledyne Isco Redisep® Gold 750 g column eluting with a 0-40% ethyl acetate/heptanes gradient to give the title compound. ¹H NMR (400 MHz, chloroform-d) δ ppm 7.79 (d, 2H), 7.35 (d, 2H), 5.82 (ddt, 1H), 5.22 (dq,), 5.16 (dq, 1H), 4.10 (dd, 1H), 4.04 (dd, 1H), 3.98 (dd, 1H), 3.94 (dt, 2H), 3.47 (dd, 1H), 3.43 (dd, 1H), 2.87 (d, 1H), 2.44 (s, 3H). MS (ESI) m/z 304.0 (M+NH₄)⁺.

### Example 12J

### (R)-tert-butyl 2-(((S)-5-((1S)-4-(((R)-1-(allyloxy)-3-(tosyloxy) propan-2-yl)oxy)-3-chloro-2-methylphenyl)-6-(4-fluorophenyl)thieno[2,3-d]pyrimidin-4-yl)oxy)-3-(2-(benzyloxy)-5-((tert-butyldimethylsilyl)oxy)phenyl)propanoate

An oven dried 250 mL 3-necked flask was charged with Example 121 (3.11 g) followed by Example 12G (5.0 g). The flask was equipped with a magnetic stir bar, sealed with rubber septa, and purged with an argon sweep for 15 minutes. Toluene (30 mL) was added and upon dissolution the flask was cooled in an ice bath to an internal temperature of 5° C. Triphenylphosphine (3.17 g) was added and the reaction mixture was stirred for 5 minutes at which point di-tert-butyl azodicarboxylate (2.78 g) was added. After 30 minutes, the cooling bath was removed and the flask was allowed to warm to ambient temperature and was stirred overnight. The reaction mixture was loaded onto a 400 mL Buchner funnel packed with silica gel which had been equilibrated with heptanes. The silica gel plug was eluted with a mixture of 1:3 ethyl acetate/heptanes (600 mL), and the solvents were concentrated. The crude product was purified by flash column chromatography on a Teledyne Isco CombiFlash® Rf instrument using a Teledyne Isco RediSep® Gold 220 g column. The desired fractions were combined and concentrated to give the title compound. ¹H NMR (400 MHz, dimethylsulfoxide-d₆) δ ppm 8.62 (s, 1H), 7.75 (d, 1H), 7.46-7.33 (m, 5H), 7.33-7.25 (m, 3H), 7.22 (t, 2H), 7.09 (d, 1H), 6.96 (d, 1H), 6.91 (d, 1H), 6.67 (dd, 1H), 6.39 (d, 1H), 5.62 (ddt, 1H), 5.31 (dd, 1H), 5.06-4.99 (m, 3H), 4.97 (dq, 1H), 4.69 (dt, 1H), 4.28 (dd, 1H), 4.18 (dd, 1H), 3.73 (dq, 2H), 3.45 (d, 2H), 2.58 (qd, 2H), 2.38 (s, 3H), 1.94 (s, 3H), 1.15 (s, 9H), 0.88 (s, 9H), 0.08 (s, 3H), 0.08 (s, 3H). MS (ESI) m/z 1095.3 (M+H)⁺.

### Example 12K

### (R)-tert-butyl 2-((5-((1S)-4-(((R)-1-(allyloxy)-3-(tosyloxy) propan-2-yl)oxy)-3-chloro-2-methylphenyl)-6-(4-fluorophenyl)thieno[2,3-d]pyrimidin-4-yl)oxy)-3-(2-(benzyloxy)-5-hydroxyphenyl)propanoate

A 100 mL round bottomed flask was charged with Example 12J (3.58 g), sealed with a septum and purged with nitrogen gas for 10 minutes. Tetrahydrofuran (23 mL) was added followed by acetic acid (0.3 mL). The stirring homogeneous solution was cooled in an ice bath to 5° C. internal temperature and a solution of tetra-N-butylammonium fluoride (4.75 mL, 1 M) in tetrahydrofuran was added dropwise. After 1 hour, the reaction mixture was quenched by the addition of a saturated solution of sodium bicarbonate (40 mL), and diluted with methyl tert-butyl ether (160 mL). The layers were separated and the organic layer was washed sequentially with water and brine, dried over MgSO₄, filtered and concentrated. The crude residue was purified by flash column chromatography on a Teledyne Isco CombiFlash® Rf instrument using a Teledyne Isco RediSep® Gold 80 g column eluting with a 0-60% ethyl acetate/heptanes gradient. The desired fractions were collected, combined and concentrated to provide the title compound. ¹H NMR (400 MHz, dimethylsulfoxide-d₆) δ ppm 8.78 (s, 1H), 8.61 (s, 1H), 7.80-7.70 (m, 2H), 7.45-7.40 (m, 2H), 7.40-7.33 (m, 4H), 7.32-7.24 (m, 3H), 7.24-7.19 (m, 2H), 7.13 (d, 1H), 7.01 (d, 1H), 6.83 (d, 1H), 6.57 (dd, 1H), 6.17 (d, 1H), 5.63 (ddt, 1H), 5.21 (dd, 1H), 5.04 (dq, 1H), 4.98 (ddt, 3H), 4.73 (dt, 1H), 4.29 (dd, 1H), 4.19 (dd, Hz, 1H), 3.75 (q, 1H), 3.74 (q, 1H), 3.48 (d, 2H), 2.59 (dd, 1H), 2.50 (d, 1H), 2.38 (s, 3H), 1.93 (s, 3H), 1.17 (s, 9H). MS (ESI) m/z 981.1 (M+H)⁺.

### Example 12L

### tert-butyl (7R,16R,21S)-10-(benzyloxy)-19-chloro-1-(4-fluorophenyl)-20-methyl-16-{[(prop-2-en-1-yl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

An oven dried 3 neck 500 mL round bottomed flask was charged with Example 12K (3.13 g), and equipped with a magnetic stir bar and sealed with rubber septa. The flask was purged with an argon flow for 10 minutes. N,N-Dimethylformamide (319 mL) was added and the material dissolved with stirring at ambient temperature. Cesium carbonate (5.19 g) was added and the suspension was stirred at ambient temperature for 3 hours. Ethyl acetate (100 mL) was added and the mixture was filtered through a pad of diatomaceous earth. The solvents were concentrated under vacuum, and the crude residue was treated with ethyl acetate (200 mL) and water (100 mL). A 1 M aqueous solution of lithium chloride was added (50 mL), and the layers were separated. The organic layer was dried over anhydrous magnesium sulfate, filtered and concentrated under reduced pressure. The crude residue was purified by flash column chromatography on a Teledyne Isco CombiFlash® Rf instrument using a Teledyne Isco RediSep® Gold 120 g column eluting with a 0-50% ethyl acetate/heptanes gradient. The desired fractions were collected, combined and concentrated to provide the title compound. ¹H NMR (400 MHz, dimethylsulfoxide-d₆) δ ppm 8.70 (s, 1H), 7.49-7.43 (m, 3H), 7.43-7.36 (m, 3H), 7.37-7.29 (m, 1H), 7.26-7.14 (m, 6H), 6.97-6.91 (m, 3H), 6.88 (dd, 1H), 5.97 (dd, 1H), 5.89 (ddt, 1H), 5.52 (d, 1H), 5.27 (dq, 1H), 5.16 (dq, 1H), 5.04 (d, 1H), 4.97 (d, 1H), 4.50 (hept, 1H), 4.46-4.41 (m, 1H), 4.41-4.37 (m, 1H), 4.06-3.97 (m, 1H), 4.01-3.92 (m, 1H), 3.76 (dd, 1H), 3.68 (dd, 1H), 3.62 (dd, 1H), 2.71 (d, 1H), 2.23 (s, 3H), 1.0fl (s, 9H). MS (ESI) m/z 809.1 (M+H)⁺.

### Example 12M

### tert-butyl (7R,16R,21S)-10-(benzyloxy)-19-chloro-1-(4-fluorophenyl)-16-(hydroxymethyl)-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

An oven dried 100 mL round bottomed flask was charged with Example 12L (2.23 g), tetrakis(triphenylphosphine)palladium (0) (0.318 g), 1,3-dimethylpyrimidine-2,4,6(1H,3H,5H)-trione (0.946 g), and a magnetic stir bar, and sealed with a septum. The flask was then purged with a flow of argon for 15 minutes. A mixture of tetrahydrofuran (18 mL) and methanol (9 mL), which was degassed by subsurface sparging with argon for 30 minutes, was added via cannula. The reaction mixture was stirred at ambient temperature for 40 hours at which point ammonium pyrrolidine-1-carbodithioate (0.181 g) was added and the stirring was continued for 1 hour. The reaction mixture was filtered through a plug of diatomaceous earth, and the filter pad was washed with ethyl acetate (25 mL) and water (25 mL). The filtrate layers were separated and the aqueous layer was extracted once with ethyl acetate (25 mL). The combined organic layers were washed with a solution of saturated aqueous sodium chloride (50 mL), dried over anhydrous magnesium sulfate, filtered and concentrated under reduced pressure. The crude residue was purified by flash column chromatography on a Teledyne Isco CombiFlash® Rf instrument using a Teledyne Isco RediSep® Gold 80 g column eluting with a 0-50% ethyl acetate/heptanes gradient. The desired fractions were collected, combined and concentrated to provide the title compound. ¹H NMR (400 MHz, dimethylsulfoxide-d₆) δ ppm 8.70 (s, 1H), 7.50-7.43 (m, 2H), 7.44-7.36 (m, 2H), 7.37-7.30 (m, 1H), 7.26-7.14 (m, 5H), 6.98-6.90 (m, 2H), 6.86 (dd, 1H), 5.96 (dd, 1H), 5.52 (d, 1H), 5.04 (d, 1H), 4.98 (q, 2H), 4.48-4.31 (m, 3H), 3.76 (dd, 1H), 3.69 (ddd, 1H), 3.56 (dt, 1H), 2.77-2.66 (m, 1H), 2.23 (s, 3H), 1.02 (s, 9H). MS (ESI) m/z 769.2 (M+H)⁺.

### Example 12N

### tert-butyl (7R,16S,21S)-10-(benzyloxy)-19-chloro-1-(4-fluorophenyl)-20-methyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

A 50 mL round bottomed flask was charged with Example 12M (1.81 g), and a magnetic stir bar. Dichloromethane was then added (16 mL), and the mixture stirred to dissolution. 1,4-Diazabicyclo[2.2.2]octane (0.660 g) and p-toluenesulfonyl chloride (0.673 g) were added sequentially. The reaction mixture was stirred at ambient temperature for 1 hour and quenched by addition of ethylenediamine (0.079 mL). The reaction mixture was stirred for 10 minutes and was diluted with dichloromethane (20 mL). A solution of 1.0 M sodium dihydrogen phosphate NaH₂PO₄ (30 mL) was added. The layers were separated and the aqueous layer was extracted with dichloromethane (20 mL). The combined organic layers were dried over anhydrous magnesium sulfate, filtered and concentrated to provide the title compound, which was used without further purification. ¹H NMR (400 MHz, dimethylsulfoxide-d₆) δ ppm 8.70 (s, 1H), 7.84-7.77 (m, 2H), 7.46 (ddd, 4H), 7.44-7.37 (m, 2H), 7.37-7.31 (m, 1H), 7.20 (d, 3H), 7.11-7.04 (m, 1H), 6.94 (d, 1H), 6.92 (d, 1H), 6.87 (dd, 1H), 5.97 (dd, 1H), 5.48 (d, 1H), 5.06 (d, 1H), 4.99 (d, 1H), 4.61-4.49 (m, 1H), 4.39-4.32 (m, 3H), 4.29 (dd, 1H), 3.75 (dd, 1H), 2.75-2.64 (m, 1H), 2.40 (s, 3H), 2.21 (s, 3H), 1.01 (s, 9H). MS (ESI) m/z 923.0 (M+H)⁺.

### Example 12O

### tert-butyl (7R,16R,21S)-10-(benzyloxy)-19-chloro-1-(4-fluorophenyl)-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

An oven dried 100 mL round bottomed flask was charged with Example 12N (2.17 g) and a magnetic stir bar, and was sealed with a rubber septum. The flask was purged with a nitrogen gas sweep for 10 minutes. N,N-Dimethylformamide (8 mL) and 1-methylpiperazine (8 mL) were added sequentially. The reaction mixture was stirred for 60 hours at ambient temperature and 16 hours at 30° C. The reaction mixture was cooled in an ice bath, and diluted with ethyl acetate (20 mL) and water (20 mL). The reaction mixture was allowed to warm to ambient temperature, and was further diluted with water (80 mL) and ethyl acetate (80 mL). The layers were separated and the aqueous layer was extracted with ethyl acetate (2×50 mL). The combined organic layers were washed sequentially with water and a 0.5 M aqueous solution of lithium chloride, dried over anhydrous magnesium sulfate, filtered, and concentrated. The crude residue was purified by flash column chromatography on a Teledyne Isco CombiFlash® Rf instrument using a Teledyne Isco RediSep® Gold 80 g column eluting with a 0-10% methanol/dichlormethane gradient to provide the title compound. ¹H NMR (501 MHz, dimethylsulfoxide-d₆) δ ppm 8.71 (s, 1H), 7.47-7.43 (m, 3H), 7.43-7.37 (m, 3H), 7.37-7.29 (m, 2H), 7.26-7.13 (m, 5H), 6.93 (d, J=2.9 Hz, 1H), 6.91 (d, J=3.7 Hz, 1H), 6.82 (dd, J=9.0, 2.9 Hz, 2H), 6.01 (dd, J=5.9, 2.3 Hz, 2H), 5.53 (d, J=2.7 Hz, 1H), 5.06 (d, J=12.1 Hz, 1H), 4.98 (d, J=12.1 Hz, 1H), 4.48 (d, J=13.2 Hz, 1H), 4.44 (dd, J=8.2, 5.5 Hz, 1H), 4.32 (dd, J=13.0, 8.4 Hz, 1H), 3.78 (dd, J=16.7, 5.9 Hz, 1H), 2.75-2.68 (m, 1H), 2.60-2.55 (m, 1H), 2.54 (dd, J=13.0, 7.8 Hz, 1H), 2.31 (d, J=29.0 Hz, 8H), 2.24 (s, 3H), 2.15 (s, 3H), 1.01 (s, 9H). MS (ESI) m/z 851.0 (M+H)⁺.

### Example 12P

### tert-butyl (7R,16R,21S)-19-chloro-1-(4-fluorophenyl)-10-hydroxy-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A 20 mL Barnstead Hastelloy C reactor was charged with palladium on carbon (0.55 g, 5% weight palladium, wet). A mixture of Example 120 (0.8 g) in tetrahydrofuran (2.5 mL) was added and the reactor was purged with argon. The mixture was stirred at 1600 rotations per minute under 50 psi of hydrogen at 25° C. for 48 hours. The solution was filtered, concentrated under reduced pressure and purified by flash column chromatography on a Teledyne Isco CombiFlash® Rf instrument using a Teledyne Isco RediSep® Gold 40 g column eluting with a 0-10% methanol/dichlormethane gradient to provide the title compound. ¹H NMR (400 MHz, dimethylsulfoxide-d₆) δ ppm 9.03 (s, 1H), 8.67 (s, 1H), 7.32-7.04 (m, 7H), 6.88 (d, 1H), 6.78-6.51 (m, 2H), 5.91 (dd, 1H), 5.33 (d, 1H), 4.43-4.32 (m, 2H), 4.24 (dd, 1H), 3.65 (dd, 1H), 2.57 (d, 1H), 2.53-2.47 (m, 3H), 2.36-2.25 (m, 8H), 2.24 (s, 3H), 2.10 (s, 3H), 1.01 (s, 9H). MS (ESI+) m/z 761.5 (M+H)⁺.

### Example 12Q

### methyl 2,3,4-tri-O-acetyl-6-O-{4-[4-({[(7R,16R,21S)-7-(tert-butoxycarbonyl)-19-chloro-1-(4-fluorophenyl)-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]inden-10-yl]oxy}methyl)pyrimidin-2-yl]phenyl}-a-D-mannopyranoside

To a mixture of Example 12P (0.060 g), Example 10D (0.080 g) and triphenylphosphine (0.043 g) in toluene (0.8 mL) under nitrogen at 0° C. was added di-tert-butyl azodicarboxylate (0.036 g) and the reaction mixture was allowed to warm to room temperature. After stirring for 7 hours, the reaction mixture was loaded onto silica gel (Teledyne Isco RediSep® Rf gold 12 g) and eluted using a gradient of 0.5-10% methanol/dichloromethane. The desired fractions were combined and the solvents were removed to provide the title compound. MS (ESI) m/z 1247.3 (M+H)⁺.

### Example 12R

### methyl 6-O-{4-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-a-D-mannopyranoside

To a mixture of Example 12Q (0.065 g) in dichloromethane (0.3 mL) was added trifluoroacetic acid (0.3 mL) and the reaction mixture was stirred at room temperature. After 6 hours, the reaction mixture was concentrated. The crude material was dissolved in dichloromethane (2 mL) and the mixture was concentrated a second time. The residue was dissolved in tetrahydrofuran (0.3 mL) and methanol (0.3 mL), treated with a solution of lithium hydroxide hydrate (0.022 g) in water (0.3 mL), and stirred for 30 minutes at room temperature. The reaction mixture was diluted with N,N-dimethylformamide (0.7 mL) and water (0.7 mL) containing 2,2,2-trifluoroacetic acid (0.040 mL). The resulting solution was purified by Prep HPLC using a Gilson 2020 system (Luna™ column, 250×50 mm, flow 70 mL/minutes) using a gradient of 5-80% acetonitrile in water (with 0.1% TFA) over 30 minutes. The desired fractions were lyophilized to provide the title compound. ¹H NMR (500 MHz, DMSO-d₆) δ ppm 8.85 (d, 1H), 8.75 (s, 1H), 8.41-8.33 (m, 2H), 7.43 (d, 1H), 7.25-7.13 (m, 4H), 7.12-7.08 (m, 2H), 6.97 (d, 1H), 6.90 (d, 1H), 6.83 (dd, 1H), 6.17 (dd, 1H), 5.68 (d, 1H), 5.25 (d, 1H), 5.18 (d, 1H), 4.59 (q, 1H), 4.55 (d, 1H), 4.47 (d, 1H), 4.42-4.31 (m, 2H), 4.15 (dd, 1H), 3.88 (dd, 1H), 3.69-3.62 (m, 2H), 3.60-3.49 (m, 9H), 3.27 (s, 3H), 3.09 (s, 4H), 2.96-2.83 (m, 2H), 2.79 (s, 3H), 2.76-2.70 (m, 2H), 2.23 (s, 3H). MS (ESI) m/z 1065.3 (M+H)⁺.

### Example 13

### (7R,16R,21S)-19-chloro-1-(4-fluorophenyl)-10-{[2-(4-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 13A 4-(4-(dimethoxymethyl)pyrimidin-2-yl)phenol

4-Hydroxybenzimidamide hydrochloride (2.5 g) was dissolved in ethanol (60 mL). Sodium ethanolate (21% in ethanol, 10.81 mL) was added, followed by (E)-4-(dimethylamino)-1,1-dimethoxybut-3-en-2-one (2.76 g). The reaction mixture was stirred at 70° C. for 16 hours. The solvent was removed by rotary evaporation. The residue was taken up in 50% ethyl acetate in heptanes (100 mL). Saturated aqueous ammonium chloride (20 mL) was added and the layers were separated. The organic layer was washed with water (2×20 mL) and with brine (20 mL). The organic layers were dried on anhydrous sodium sulfate, and filtered. The mixture was concentrated and was allowed to stand for 16 hours. The material was filtered out, washed with diethyl ether and dried under vacuum to provide the title compound. ¹H NMR (400 MHz, DMSO-d₆) δ ppm 8.86 (d, 1H), 9.98 (bs, 1H), 8.25 (d, 2H), 7.35 (d, 1H), 6.89 (d, 2H), 5.32 (s, 1H), 3.38 (s, 6H). MS (ESI) m/z 245 (M−H)⁻.

### Example 13B

### 4-(dimethoxymethyl)-2-(4-(2-(2-(2-methoxyethoxy)ethoxy)ethoxy)phenyl)pyrimidine

Example 13A (4.994 g) and 2-(2-(2-methoxyethoxy)ethoxy)ethanol (4.10 mL) were dissolved in tetrahydrofuran (100 mL). Triphenylphosphine (6.38 g) was added, and the mixture was stirred until it dissolved. (E)-Diisopropyl diazene-1,2-dicarboxylate (4.79 mL) was added, and the mixture was stirred for 16 hours at room temperature. The mixture was concentrated under vacuum and purified by flash column chromatography on silica gel using a gradient of 30-70% ethyl acetate in heptanes. The solvent was removed from the desired fractions by rotary evaporation to provide the title compound. ¹H NMR (400 MHz, DMSO-d₆) δ ppm 8.89 (d, 1H), 8.35 (d, 2H), 7.39 (d, 1H), 7.09 (d, 2H), 5.34 (s, 1H), 4.19 (t, 2H), 3.78 (t, 2H), 3.62-3.59 (m, 4H), 3.56-3.50 (m, 4H), 3.44-3.42 (m, 2H), 3.39 (s, 6H), 3.24 (s, 3H). MS (ESI) m/z 393 (M+H)⁺.

### Example 13C

### (2-(4-(2-(2-(2-methoxyethoxy)ethoxy)ethoxy)phenyl)pyrimidin-4-yl)methanol

Example 13B (7.503 g) was dissolved in 1,4-dioxane (80 mL). Aqueous hydrogen chloride (2 M, 80 mL) was added and the mixture was heated to 50° C. for 16 hours. The mixture was cooled to room temperature and further cooled to 0° C. using an ice bath. The pH of the mixture was adjusted to eight using concentrated aqueous sodium hydroxide. To the mixture was added sodium tetrahydroborate (1.446 g) in three portions five minutes apart. The mixture was stirred at 0° C. for one hour. While keeping the reaction at 0° C. 20 mL of ethyl acetate was added, and the mixture was stirred for 10 minutes. The mixture was diluted further with ethyl acetate (20 mL). The phases were separated. The aqueous layer was extracted with ethyl acetate (25 mL) once. The organic portions were combined, dried on anhydrous sodium sulfate and filtered. The mixture was concentrated under vacuum and purified by flash column chromatography on silica gel using 100% ethyl acetate. The solvent was removed by rotary evaporation to provide the title compound. ¹H NMR (400 MHz, DMSO-d₆) δ ppm 8.82 (d, 1H), 8.33 (d, 2H), 7.41 (d, 1H), 7.07 (d, 2H), 5.65 (t, 1H), 4.62 (d, 2H), 4.17 (t, 2H), 3.77 (t, 2H), 3.61-3.59 (m, 2H), 3.55-3.51 (m, 4H), 3.44-3.42 (m, 2H), 3.23 (s, 1H). MS (ESI) m/z 349 (M+H)⁺.

### Example 13D

### tert-butyl (7R,16R,21S)-19-chloro-1-(4-fluorophenyl)-10-{[2-(4-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 13C (63 mg), Example 12P (60 mg), and triphenylphosphine (43 mg) were dissolved in toluene (0.8 mL). The mixture was cooled to 0° C. using an ice bath. (E)-di-tert-butyl diazene-1,2-dicarboxylate (36 mg) was added. The reaction mixture was allowed to warm to room temperature and stir for 16 hours. Additional Example 13C (63 mg), triphenylphosphine (43 mg) and (E)-di-tert-butyl diazene-1,2-dicarboxylate (36 mg) were added, and the reaction mixture was stirred another 24 hours at room temperature. The mixture was purified by flash column chromatography on silica gel using a gradient of 0-10% methanol in dichloromethane. The solvent was removed by rotary evaporation to provide the title compound. ¹H NMR (400 MHz, DMSO-d₆) δ ppm 8.89 (d, 1H), 8.73 (s, 1H), 8.37 (d, 2H), 7.46 (d, 1H), 7.25-7.16 (m, 5H), 7.09 (d, 2H), 6.94 (dd, 2H), 6.83 (dd, 1H), 6.07 (dd, 1H), 5.57 (d, 1H), 5.21 (q, 2H), 4.48 (d, 1H), 4.43 (m, 1H), 4.33 (dd, 1H), 4.18 (t, 2H), 3.89 (dd, 2H), 3.78 (t, 2H), 3.63-3.59 (m, 2H), 3.54 (m, 4H), 3.45-3.42 (m, 2H), 3.23 (s, 2H), 2.83 (d, 1H), 2.59-2.52 (m, 4H), 2.40-2.27 (m, 6H), 2.26 (s, 3H), 2.12 (s, 3H), 1.02 (s, 9H). MS (ESI) m/z 1091 (M+H)⁺.

### Example 13E

### (7R,16R,21S)-19-chloro-1-(4-fluorophenyl)-10-{[2-(4-{2-[2-(2-methoxyethoxy)ethoxy]ethoxy}phenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 13D (42 mg) was dissolved in dichloromethane (0.25 mL). Trifluoroacetic acid (0.25 mL) was added and the mixture was stirred at room temperature. After six hours, the solvents were removed under vacuum. The residue was taken up in N,N-dimethylformamide (1 mL) and water (1 mL). The material was purified by reverse phase using a 30-100% gradient of acetonitrile in water (with 0.1% trifluoroacetic acid) over 40 minutes on a Grace Reveleris equipped with a Luna™ column: C18(2), 100 Å, 250×50 mm. The product fractions were pooled, frozen and lyophilized to isolate the title compound as the bis trifluoroacetic acid salt. ¹H NMR (400 MHz, DMSO-d₆) δ ppm 9.41 (bs, 1H), 8.85 (d, 1H), 8.75 (s, 1H), 8.35 (d, 1H), 7.43 (d, 1H), 7.22-7.18 (m, 4H), 7.15 (d, 2H), 7.10 (d, 2H), 6.96 (d, 1H), 6.90 (d, 1H), 6.83 (dd, 1H), 6.16 (m, 1H), 5.67 (d, 1H), 5.21 (q, 2H), 4.58 (m, 1H), 4.47 (d, 1H), 4.36 (dd, 1H), 4.19 (t, 2H), 3.88 (dd, 2H), 3.78 (t, 2H), 3.62-3.59 (m, 2H), 3.56-3.51 (m, 6H), 3.44-3.41 (m, 2H), 3.24 (s, 2H), 3.13-2.97 (m, 3H), 2.95-2.83 (m, 2H), 2.78 (s, 3H), 2.74-2.66 (m, 2H), 2.48-2.32 (m, 2H), 2.22 (s, 3H). MS (ESI) m/z 1035 (M+H)⁺.

### Example 14

### methyl 6-O-{4-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

### Example 14A

### methyl 6-O-{4-[4-({[(7R,16R,21S)-7-(tert-butoxycarbonyl)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

The title compound was prepared by substituting Example 11B for Example 10D in Example 12Q. MS (ESI) m/z 1163.1 (M+H)⁺.

### Example 14B

### methyl 6-O-{4-[4-({[(7R,16R,21S)-7-carboxy-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]inden-10-yl]oxy}methyl)pyrimidin-2-yl]phenyl}-2,3,4-tri-O-methyl-a-D-mannopyranoside

The title compound was prepared by substituting Example 14A for Example 12Q in Example 12R. ¹H NMR (500 MHz, dimethylsulfoxide-d₆) δ ppm 8.85 (d, 1H), 8.74 (s, 1H), 8.36 (d, 2H), 7.43 (d, 1H), 7.20 (m, 4H), 7.13 (m, 3H), 6.92 (d, 1H), 6.90 (d, 1H), 6.82 (dd, 1H), 6.17 (m, 1H), 5.67 (d, 1H), 5.25 (d, 1H), 5.17 (d, 1H), 4.79 (s, 1H), 4.57 (m, 1H), 4.46 (d, 1H), 4.35 (m, 1H), 4.21 (m, 2H), 3.89 (dd, 1H), 3.65 (v br m, 1H), 3.61 (br m, 1H), 3.45 (m, 5H), 3.40 (s, 3H), 3.39 (s, 3H), 3.36 (s, 3H), 3.30 (s, 3H), 3.07 (v br s, 3H), 2.91 (br d, 2H), 2.78 (s, 3H), 2.73 (br m, 2H), 2.41 (v br s, 1H), 2.22 (s, 3H). MS (ESI) m/z 1107.4 (M+H)⁺.

### Example 15

### (7R,16R,21S)-19,23-dichloro-1-(4-fluorophenyl)-10-{[2-(2-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 15A

### thieno[2,3-d]pyrimidin-4(3H)-one

A mixture of 2-amino-3-cyanothiophene (50 g) in formic acid (100 mL) and H₂SO₄ (22 mL) was heated in a sealed tube for 2 hours at 100° C. The mixture was cooled to 20° C. and was diluted with water (1 L). The resulting precipitate was collected by filtration, washed with water twice (2×1 L) and dried under reduced pressure to provide the title compound. ¹H NMR (400 MHz, dimethyl sulfoxide-d₆) δ ppm 12.16 (br. s., 1H), 8.09 (s, 1H), 7.54 (d, J=5.6 Hz, 1H), 7.35 (d, J=6.0 Hz, 1H).

### Example 15B

### 5,6-diiodothieno[2,3-d]pyrimidin-4(3H)-one

To an ice-cooled 4-neck 2 L flask fit with a mechanical stirrer, reflux condenser and thermocouple/JKEM was added acetic acid (160 mL), sulfuric acid (8 mL) and water (80 mL) with stirring. Example 15A (40.0 g), periodic acid (30.0 g) and iodine (133 g) were added sequentially and the mixture became slightly endothermic. The ice bucket was removed and a heating mantle was added. The reaction mixture was ramped up to 60° C. and was stirred for 20 minutes. The temperature climbed to 95° C. The heating mantle was removed and reaction mixture was allowed to cool to room temperature. The resulting suspension was poured into saturated aqueous sodium sulfite solution, filtered, and washed with water. The organic layer was dried under vacuum to provide the title compound.

### Example 15C

### 4-chloro-5,6-diiodothieno[2,3-d]pyrimidine

A 250 mL flask equipped with magnetic stirring, heating mantle, temperature probe and reflux condenser to a nitrogen bubbler was charged with phosphorus oxychloride (57.3 mL) and N,N-dimethylaniline (17.64 mL). To the mixture was added Example 15B (56.22 g) over 5 minutes. The resulting suspension was heated at 105° C. for 30 minutes. After cooling, the resulting material was broken up and transferred to a funnel with heptane. The material was washed with heptane to remove most of the phosphorus oxychloride. The material was slowly scooped into rapidly stirring ice water (600 mL) and stirred for 30 minutes. The material was collected by filtration, washed with water and ether (200 mL), dried over Na₂SO₄, and filtered to provide the title compound which was used in the next step without further purification.

### Example 15D

### 4-chloro-5-iodothieno[2,3-d]pyrimidine

A 500 mL 3-neck jacketed flask with magnetic stirring under nitrogen was charged with Example 15C (23 g) and tetrahydrofuran (200 mL). The resulting suspension was cooled to −16° C. using a Huber chiller set to −17° C. To the mixture was added tert-butylmagnesium chloride (40.8 mL, 2 M in ether) dropwise over 40 minutes, keeping the temperature between −15° C. and −16° C. The temperature was slowly raised to 0° C. and was stirred for 30 minutes. The reaction mixture was cooled to −20° C. and was quenched by the very slow dropwise addition (initially about 1 drop/minute) of water (23 mL) over 35 minutes, maintaining the temperature at about −20° C., and then slowly warmed to ambient temperature over 1 hour. The stirring was stopped and the supernatant was decanted from the remaining residue. To the residue was added tetrahydrofuran (200 mL). The mixture was stirred briefly, and after standing, the supernatant was decanted from the remaining residue. This was repeated two times. The combined organics were concentrated. The crude material was purified by chromatography on silica gel eluting with isocratic methylene chloride. The title compound was precipitated from a minimum of hot heptanes.

### Example 15E

### 4-chloro-5-(4-methoxy-2,6-dimethylphenyl)thieno[2,3-d]pyrimidine

To a suspension of Example 15D (5 g). (4-methoxy-2,6-dimethylphenyl)boronic acid (6.07 g) and cesium carbonate (10.99 g) in degassed toluene (50.0 mL) and water (12.5 mL) was added bis(di-tert-butyl (4-dimethylaminophenyl)phosphine)dichloropalladium (II) (597 mg). The mixture was heated to 100° C. overnight. After cooling to room temperature, the mixture was diluted with ethyl acetate (200 mL). The organic layer was washed with water and brine, dried over anhydrous sodium sulfate, filtered and concentrated under vacuum. The residue was purified by silica gel chromatography on a CombiFlash® Teledyne Isco system eluting with 0-20% ethyl acetate in heptanes to provide the title compound. ¹H NMR (501 MHz, CDCl₃) δ ppm 8.88 (s, 1H), 7.35 (s, 1H), 6.70 (s, 2H), 3.85 (s, 3H), 1.99 (s, 6H). MS (ESI) m/z 305.1 (M+H)⁺.

### Example 15F

### 4-chloro-6-iodo-5-(4-methoxy-2,6-dimethylphenyl)thieno[2,3-d]pyrimidine

To a mixture of diisopropylamine (4.15 mL) in tetrahydrofuran (50 mL) cooled to −78° C. was added n-butyllithium (9.71 mL, 2.5 M in hexanes) dropwise. The mixture was stirred for 1 minute before Example 15E (3.7 g) was added as a mixture in tetrahydrofuran (50 mL). The resulting mixture was stirred at −78° C. for 15 minutes. Iodine (6.16 g) was added in one portion and the mixture was warmed to room temperature. The reaction mixture was quenched with saturated aqueous ammonium chloride mixture (100 mL) and was extracted with ethyl acetate (50 mL×3). The combined organic layers were washed sequentially with a sodium thiosulfate mixture and brine, dried over anhydrous sodium sulfate, filtered and concentrated onto silica gel. Purification by flash chromatography on a silica gel column eluting with 0-20% ethyl acetate in heptanes provided crude product, which was triturated with heptanes to obtain the title compound. ¹H NMR (501 MHz, CDCl₃) δ ppm 8.82 (s, 1H), 6.72 (s, 2H), 3.87 (s, 3H), 1.94 (s, 6H). MS (ESI) m/z 431.1 (M+H)⁺.

### Example 15G

### 4-chloro-6-(4-fluorophenyl)-5-(4-methoxy-2,6-dimethylphenyl)thieno[2,3-d]pyrimidine

To a mixture of Example 15F (3.3 g), (4-fluorophenyl)boronic acid (2.144 g) di-tert-butyl (2′,4′,6′-triisopropyl-[1,1′-biphenyl]-2-yl)phosphine (0.179 g) and potassium phosphate tribasic (3.25 g) in degassed tetrahydrofuran (60 mL) and water (15 mL) was added tris(dibenzylideneacetone) dipalladium (0) (0.175 g). The mixture was heated to 60° C. overnight. After cooling to room temperature, the mixture was diluted with ethyl acetate (100 mL). The organic layer was washed with brine, dried over anhydrous sodium sulfate, filtered and concentrated under vacuum. The residue was purified by flash chromatography on a silica gel column eluting with 0-20% ethyl acetate in heptanes to give crude product, which was triturated with heptanes to obtain the title compound. ¹H NMR (501 MHz, CDCl₃) δ ppm 8.84 (s, 1H), 7.31-7.23 (m, 2H), 7.02-6.93 (m, 2H), 6.65 (d, 2H), 3.83 (s, 3H), 1.92 (d, 6H). MS (ESI) m/z 399.1 (M+H)⁺.

### Example 15H

### 4-chloro-5-(3,5-dichloro-4-methoxy-2,6-dimethylphenyl)-6-(4-fluorophenyl)thieno[2,3-d]pyrimidine

To a suspension of Example 15G (2.13 g) in acetonitrile (50 mL) was added N-chlorosuccinimide (2.85 g). The mixture was heated to reflux for 1 hour. The mixture was concentrated under vacuum and the residue was redissolved in ethyl acetate (50 mL). The mixture was washed with brine, dried over anhydrous sodium sulfate, filtered and concentrated under vacuum. The residue was purified by silica gel chromatography on a CombiFlash® Teledyne Isco system eluting with 0-100% ethyl acetate in heptanes to provide the title compound. ¹H NMR (400 MHz, CDCl₃) δ ppm 8.89 (s, 1H), 7.28-7.18 (m, 2H), 7.08-6.97 (m, 2H), 3.96 (s, 3H), 2.02 (s, 6H). MS (ESI) m/z 469.1 (M+H)⁺.

### Example 15I

### 2,6-dichloro-4-(4-chloro-6-(4-fluorophenyl)thieno[2,3-d]pyrimidin-5-yl)-3,5-dimethylphenol

To Example 15H (5 g) in 1,2-dichloroethane (200 mL) was added aluminum trichloride (4.28 g), and the mixture was heated to 68° C. for 6 hours and cooled to room temperature. Saturated aqueous NaHCO₃ (3 mL) was added and the mixture was stirred for 2 minutes. Saturated aqueous NH₄Cl (15 mL) was added. The mixture was diluted with ethyl acetate and the layers were separated. The aqueous layer was extracted once with ethyl acetate. The organic layers were combined and washed with water and brine, dried over Na₂SO₄, filtered, and concentrated to provide the title compound. ¹H NMR (400 MHz, dimethylsulfoxide-d₆) δ ppm 10.10 (br s, 1H), 9.00 (s, 1H), 7.35 (m, 2H), 7.28 (m, 2H), 1.96 (s, 6H). MS (ESI) m/z 452.9 (M−H)⁻.

### Example 15J

### (S)-1-(allyloxy)-3-(bis(4-methoxyphenyl) (phenyl)methoxy) propan-2-ol

To a mixture of Example 12H (2.25 g) and 4,4′-(chloro(phenyl)methylene)bis(methoxybenzene) (DMTrCl) (6.06 g) in dichloromethane (68.1 mL) cooled to 0° C. was added N,N-diisopropylethylamine (3.27 mL). The mixture was allowed to warm to room temperature and was stirred for 30 minutes. The reaction mixture was quenched with saturated aqueous ammonium chloride mixture (50 mL). The organic layer was washed with brine, dried over anhydrous sodium sulfate, filtered and concentrated under vacuum. The residue was purified by silica gel chromatography on a CombiFlash® Teledyne Isco system, eluting with 0-50% ethyl acetate in heptanes to provide the title compound. ¹H NMR (400 MHz, CDCl₃) δ ppm 7.45-7.40 (m, 2H), 7.35-7.24 (m, 6H), 7.24-7.17 (m, 1H), 6.86-6.77 (m, 4H), 5.95-5.79 (m, 1H), 5.24 (dq, 1H), 5.17 (dq, 1H), 4.00 (dt, 2H), 3.98-3.91 (m, 1H), 3.78 (s, 6H), 3.55 (dd, 1H), 3.49 (dd, 1H), 3.24-3.16 (m, 2H), 2.40 (bs, 1H). MS (ESI) m/z 457.1 (M+Na)⁺.

### Example 15K

### (R)-5-(4-((1-(allyloxy)-3-(bis(4-methoxyphenyl) (phenyl)methoxy) propan-2-yl)oxy)-3,5-dichloro-2,6-dimethylphenyl)-4-chloro-6-(4-fluorophenyl)thieno[2,3-d]pyrimidine

Triphenylphosphine (1.561 g). Example 151 (1.5 g), and Example 15J (1.580 g) were taken up in 18 mL tetrahydrofuran and di-tert-butylazodicarboxylate (1.370 g) was added and the reaction was stirred overnight. The material was filtered off and rinsed with 1:1 ether/ethyl acetate, and the organics were concentrated. The crude material was chromatographed on silica gel using 1-40% ethyl acetate in heptanes as eluent to provide the title compound. MS (ESI) m/z 891.1 (M+Na)⁺.

### Example 15L

### ethyl (R)-2-((5-((1S)-4-(((R)-1-(allyloxy)-3-(bis(4-methoxyphenyl) (phenyl)methoxy) propan-2-yl)oxy)-3,5-dichloro-2,6-dimethylphenyl)-6-(4-fluorophenyl)thieno[2,3-d]pyrimidin-4-yl)oxy)-3-(5-((tert-butyldimethylsilyl)oxy)-2-((2-(2-(2-(2-(2-methoxyethoxy)ethoxy)ethoxy)phenyl)pyrimidin-4-yl)methoxy)phenyl)propanoate

Example 1H (1.07 g). Example 15K (1.527 g) and cesium carbonate (883 mg) were heated in anhydrous tert-butyl alcohol (10 mL) at 65° C. for 18 hours. The mixture was cooled and was diluted with ethyl acetate. The mixture was vacuum filtered over a pad of diatomaceous earth. The filtrate was washed with water, and brine, dried over anhydrous sodium sulfate, and filtered. The filtrate was concentrated under vacuum and was purified by flash column chromatography on silica gel using a gradient of 10-100% ethyl acetate in heptanes to provide the title compound. LCMS (APCI) m/z 1504.3 (M+H)⁺.

### Example 15M

### (R)-ethyl 2-((5-((1S)-4-(((S)-1-(allyloxy)-3-hydroxypropan-2-yl)oxy)-3,5-dichloro-2,6-dimethylphenyl)-6-(4-fluorophenyl)thieno[2,3-d]pyrimidin-4-yl)oxy)-3-(5-((tert-butyldimethylsilyl)oxy)-2-((2-(2-(2-(2-(2-methoxyethoxy)ethoxy)ethoxy)phenyl)pyrimidin-4-yl)methoxy)phenyl)propanoate

Example 15L (1.1 g) was stirred in 5 mL dichloroethane and 5 mL methanol at 0° C. To the mixture was added formic acid (3.80 mL) and the reaction mixture was stirred at 0° C. for 15 minutes. Thin layer chromotography showed the reaction was complete. The reaction mixture was diluted with 7 mL water, and solid NaHCO₃ was added slowly until pH 7-8 was reached. The mixture was extracted with dichloromethane, washed with brine, dried over Na₂SO₄, filtered and concentrated. The crude material was purified by flash column chromatography on silica gel using a gradient of 10-100% ethyl acetate in heptanes, followed by 5% methanol in ethyl acetate to provide the title compound. LCMS (APCI) m/z 1203.4 (M+H)⁺.

### Example 15N

### (R)-ethyl 2-((5-((1S)-4-(((R)-1-(allyloxy)-3-(tosyloxy) propan-2-yl)oxy)-3,5-dichloro-2,6-dimethylphenyl)-6-(4-fluorophenyl)thieno[2,3-d]pyrimidin-4-yl)oxy)-3-(5-((tert-butyldimethylsilyl)oxy)-2-((2-(2-(2-(2-(2-methoxyethoxy)ethoxy)ethoxy)phenyl)pyrimidin-4-yl)methoxy)phenyl)propanoate

To a solution of Example 15M (600 mg) and 1,4-diazabicyclo[2.2.2]octane (112 mg) in 7 mL dichloromethane at 0° C. was added TsCl (p-toluenesulfonyl chloride) (105 mg). The mixture was stirred at room temperature for 24 hours. The mixture was diluted with ethyl acetate, washed with pH 7 buffer, and concentrated. The crude material was purified by flash column chromatography on silica gel using a gradient of 10-80% ethyl acetate in heptanes to provide the title compound. ¹H NMR (400 MHz, DMSO-d₆) δ ppm 8.82 (d, 1H), 8.63 (s, 1H), 7.69 (m, 2H), 7.55 (d, 1H), 7.43 (m, 4H), 7.32 (m, 2H), 7.20 (m, 3H), 7.04 (t, 1H), 6.87 (d, 1H), 6.67 (m, 1H), 6.44 (d, 1H), 5.58 (m, 2H), 5.11 (s, 2H), 5.07 (m, 2H), 4.51 (m, 1H), 4.28 (m, 2H), 4.11 (m, 2H), 3.95 (m, 2H), 3.70 (m, 2H), 3.64 (m, 2H), 3.55 (m, 2H), 3.47 (m, 2H), 3.40 (m, 4H), 3.33 (m, 2H), 3.17 (s, 3H), 2.86 (m, 1H), 2.51 (m, 1H), 2.37 (s, 3H), 2.07 (s, 3H), 1.85 (s, 3H), 0.95 (t, 3H), 0.86 (s, 9H), 0.05 (s, 6H). LCMS (APCI) m/z 1357.4 (M+H)⁺.

### Example 150

### (R)-ethyl 2-((5-((1S)-4-(((R)-1-(allyloxy)-3-(tosyloxy) propan-2-yl)oxy)-3,5-dichloro-2,6-dimethylphenyl)-6-(4-fluorophenyl)thieno[2,3-d]pyrimidin-4-yl)oxy)-3-(5-hydroxy-2-((2-(2-(2-(2-(2-methoxyethoxy)ethoxy)ethoxy)phenyl)pyrimidin-4-yl)methoxy)phenyl)propanoate

To a mixture of Example 15N (670 mg) in 15 mL tetrahydrofuran was added TBAF (tetra-N-butylammonium fluoride) (494 μL), and the mixture was stirred at room temperature for 20 minutes. The mixture was diluted with ethyl acetate, washed with pH 7 buffer, and concentrated. The crude material was purified by flash column chromatography on silica gel using a gradient of 10-80% ethyl acetate in heptanes to provide the title compound. ¹H NMR (400 MHz, DMSO-d₆) δ ppm 8.89 (s, 1H), 8.87 (d, 1H), 8.64 (s, 1H), 7.69 (m, 2H), 7.57 (d, 1H), 7.44 (m, 4H), 7.31 (m, 2H), 7.21 (m, 3H), 7.05 (t, 1H), 6.81 (d, 1H), 6.57 (m, 1H), 6.14 (d, 1H), 5.65 (m, 1H), 5.45 (m, 1H), 5.08 (s, 2H), 5.02 (m, 2H), 4.52 (m, 1H), 4.25 (m, 2H), 4.12 (m, 2H), 4.00 (m, 2H), 3.72 (m, 2H), 3.68 (m, 2H), 3.58 (m, 2H), 3.48 (m, 2H), 3.42 (m, 4H), 3.33 (m, 2H), 3.17 (s, 3H), 2.92 (m, 1H), 2.44 (m, 1H), 2.37 (s, 3H), 2.15 (s, 3H), 1.85 (s, 3H), 1.00 (t, 3H). LCMS (APCI) m/z 1243.6 (M+H)⁺.

### Example 15P

### ethyl (7R,16R,21S)-19,23-dichloro-1-(4-fluorophenyl)-10-{[2-(2-{2-[2-(2-methoxyethoxy)ethoxy]ethoxy}phenyl)pyrimidin-4-yl]methoxy}-20,22-dimethyl-16-{[(prop-2-en-1-yl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a solution of Example 150 (565 mg) in 35 mL N,N-dimethylformamide was added cesium carbonate (741 mg), and the mixture was stirred at room temperature for 1 hour. The mixture was poured into 500 mL water and extracted with 5×200 mL ethyl acetate. The organic extracts were combined, rinsed with water and brine, dried over Na₂SO₄, filtered, and concentrated. The crude material was purified by flash column chromatography on silica gel using a gradient of 10-80% ethyl acetate in heptanes to provide the title compound. LCMS (APCI) m/z 1069.5 (M+H)⁺.

### Example 15Q

### ethyl (7R,16R,21S)-19,23-dichloro-1-(4-fluorophenyl)-16-(hydroxymethyl)-10-{[2-(2-{2-[2-(2-methoxyethoxy)ethoxy]ethoxy}phenyl)pyrimidin-4-yl]methoxy}-20,22-dimethyl-7,8,15,16-tetrahydro-18,21-etheno-9,13-(metheno)-6,14,17-trioxa-2-thia-3,5-diazacyclononadeca[1,2,3-cd]indene-7-carboxylate

To a degassed solution of Example 15P (410 mg) in 6 mL tetrahydrofuran and 3 mL methanol was added Pd (PPh₃)₄ (tetrakis(triphenylphosphine)palladium (0), 44.3 mg) and 1,3-dimethylpyrimidine-2,4,6(1H,3H,5H)-trione (150 mg), and the mixture was stirred at room temperature overnight. Ammonium pyrrolidine-1-carbodithioate (100 mg) was added and the reaction mixture was stirred for 30 minutes. The mixture was filtered through diatomaceous earth and rinsed with ethyl acetate. The organics were concentrated. The crude material was purified by flash column chromatography on silica gel using a gradient of 10-00% ethyl acetate in heptanes, and then 5% methanol in ethyl acetate, to provide the title compound. LCMS (APCI) m/z 1029.5 (M+H)⁺.

### Example 15R

### ethyl (7R,16S,21S)-19,23-dichloro-1-(4-fluorophenyl)-10-{[2-(2-{2-[2-(2-methoxyethoxy)ethoxy]ethoxy}phenyl)pyrimidin-4-yl]methoxy}-20,22-dimethyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a solution of Example 15Q (250 mg) and DABCO (1,4-diazabicyclo[2,2,2]octane) (54.5 mg) in 6 mL dichloromethane at 0° C. was added p-toluenesulfonyl chloride (55.5 mg) and the mixture was stirred at room temperature for 5 days. The crude mixture was purified by flash column chromatography on silica gel using a gradient of 10-00% ethyl acetate in heptanes, and then 5% methanol in ethyl acetate, to provide the title compound. LCMS (APCI) m/z 1185.5 (M+H)⁺.

### Example 15S

### ethyl (7R,16R,21S)-19,23-dichloro-1-(4-fluorophenyl)-10-{[2-(2-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A solution of Example 15R (250 mg) and 1-methylpiperazine (634 mg) in 1.4 mL N,N-dimethylformamide was stirred at 38° C. for 3 days. The mixture was cooled and poured into 200 mL ethyl acetate, washed three times with water and brine, dried over Na₂SO₄, filtered, and concentrated. The crude mixture was purified by flash column chromatography on silica gel using a gradient of 10-100% ethyl acetate in heptanes, then 5% methanol in ethyl acetate, and finally 5% methanol in ethyl acetate with 1% trimethylamine, to provide the title compound. LCMS (APCI) m/z 1111.3 (M+H)⁺.

### Example 15T

### (7R,16R,21S)-19,23-dichloro-1-(4-fluorophenyl)-10-{[2-(2-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

A 1M aqueous solution of LiOH (611 μL) was added to Example 15S (170 mg) in 1.8 mL tetrahydrofuran and 0.8 mL methanol and the reaction was stirred overnight. The reaction mixture was quenched by the addition of 200 μL trifluoroacetic acid and 1 mL N,N-dimethylformamide, and the mixture was subjected to vacuum to remove volatiles. The crude material was purified by reverse phase chromatography using a 20-80% gradient of acetonitrile in water (with 0.1% ammonium acetate) over 45 minutes on a Grace Reveleris equipped with a Luna™ column: C18(2), 100 Å, 250×50 mm, to provide the title compound. ¹H NMR (400 MHz, DMSO-d₆) δ ppm 8.86 (d, 1H), 8.72 (s, 1H), 7.57 (m, 2H), 7.44 (dd, 1H), 7.22-7.12 (m, 5H), 7.07 (t, 1H), 6.85 (d, 1H), 6.73 (d, 1H), 6.18 (m, 1H), 5.87 (d, 1H), 5.17 (q, 2H), 4.91 (m, 1H), 4.45 (d, 2H), 4.13 (t, 2H), 3.68 (t, 2H), 3.50 (m, 2H), 3.44 (m, 4H), 3.35 (m, 42), 3.19 (s, 3H), 2.91 (d, 2H), 2.67 (m, 4H), 2.46 (m, 2H), 2.33 (m, 4H), 2.16 (s, 3H), 2.00 (s, 3H), 1.95 (s, 3H). LCMS (APCI) m/z 1085.6 (M+H)⁺.

### Example 16

### (7R,16R)-19,23-dichloro-1-(4-fluorophenyl)-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 16A

### 2-(benzyloxy)-5-((tert-butyldimethylsilyl)oxy)benzaldehyde

A 2 L round bottom flask was charged with 2,5-dihydroxybenzaldehyde (30 g), imidazole (29.6 g) and dichloromethane (543 mL). The flask was placed in a water bath and solid tert-butylchlorodimethylsilane (32.7 g) was added. The reaction mixture was stirred at ambient temperature for 15 minutes at which point thin-layer chromatography indicated complete consumption of starting material. The reaction mixture was poured into a separatory funnel with 200 mL water. The biphasic mixture was shaken and the layers were separated. The aqueous layer was washed with 100 mL of dichloromethane and the organic layers were combined. The organic layer was dried over sodium sulfate, filtered, and concentrated to provide 5-((tert-butyldimethylsilyl)oxy)-2-hydroxybenzaldehyde.

A 1 L three-necked round bottom flask equipped with an internal temperature probe, a reflux condenser, and a stir bar was charged with 5-((tert-butyldimethylsilyl)oxy)-2-hydroxybenzaldehyde (45 g, 178 mmol) in acetone (297 mL). Solid K₂CO₃ (27.1 g) was added followed by dropwise addition of neat benzyl bromide (21.21 mL). The mixture was stirred at ambient temperature for 10 minutes and heated to 55° C. The reaction mixture was stirred overnight. The reaction mixture was cooled to ambient temperature then poured over cold water (200 mL). The mixture was then transferred to a 1 L separatory funnel. The crude product was extracted with ethyl acetate (3×250 mL). The combined organic layers were dried over sodium sulfate, filtered, and concentrated. The crude material was purified by silica gel chromatography over a 330 g column on a Grace Reveleris system (0-5% ethyl acetate/heptanes elution gradient). Fractions containing the desired product were combined, concentrated and dried under vacuum to provide the title compound. ¹H NMR (501 MHz, dimethyl sulfoxide-d₆) δ ppm 10.35 (s, 1H), 7.51-7.47 (m, 2H), 7.42-7.37 (m, 2H), 7.35-7.31 (m, 1H), 7.22 (d, 1H), 7.15 (dd, 1H), 7.11 (d . . . 1H), 5.21 (s, 2H), 0.93 (s, 10H), 0.16 (s, 7H).

### Example 16B

### tert-butyl 2-acetoxy-2-(diethoxyphosphoryl)acetate

A 3 L jacketed round bottom flask equipped with an overhead stirrer was charged with glyoxylic acid monohydrate (15 g) and diethyl phosphite (20.82 mL) and was heated to a 60° C. jacket temperature with stirring. The flask headspace was continuously purged with a nitrogen sweep. After stirring overnight, dichloromethane (250 mL) was added, the reaction was cooled to an internal temperature of 5° C. Pyridine (13.05 mL) was added dropwise. After stirring for 1 hour at 5° C. acetyl chloride (11.47 mL) was added dropwise over 20 minutes. The reaction was warmed to 20° C. stirred for 1.5 hours, and cooled to 5° C. internal temperature. Pyridine (19.57 mL) was added slowly, tert-Butanol (15.43 mL) was added in one portion followed by dropwise addition of 2,4,6-tripropyl-1,3,5,2,4,6-trioxatriphosphinane 2,4,6-trioxide (144 mL, 50% by weight in ethyl acetate) over 20 minutes. After stirring for 1 hour, the reaction was warmed to 20° C. and was stirred overnight. The reactor was then cooled to 5° C. and 1 N aqueous hydrochloric acid (200 mL) was added slowly. The biphasic mixture was stirred for 30 minutes at 20° C. and was poured into a separatory funnel. Dichloromethane (400 mL) and 1 N aqueous hydrochloric acid (250 mL) were added and the mixture was separated. The aqueous layer was extracted with dichloromethane (400 mL), and the combined organic layers were washed with a mixture of water (300 mL) and saturated aqueous sodium chloride solution (300 mL). The combined organics were dried over anhydrous magnesium sulfate, filtered and concentrated under reduced pressure. The crude residue was purified by plug filtration on silica gel eluting with 1:1 ethyl acetate/heptanes. The title compound was provided after concentration of the desired fractions under reduced pressure. ¹H NMR (400 MHz, Chloroform-d) δ ppm 5.32 (d, 1H), 4.29-4.18 (m, 4H), 2.21 (s, 3H), 1.37 (tdd, 6H). MS (ESI) m/z 255.0 (M-tert-butyl+2H)⁺.

### Example 16C

### (E)-tert-butyl 2-acetoxy-3-(2-(benzyloxy)-5-((tert-butyldimethylsilyl)oxy)phenyl)acrylate

An oven dried 2 L 3-neck round bottomed flask equipped with overhead stirring was charged with anhydrous lithium chloride (5.55 g). The flask was purged with a sweep of argon for 10 minutes and anhydrous tetrahydrofuran (350 mL) was added. A solution of Example 16B (40.6 g) in tetrahydrofuran (50 mL) was added, A solution of 1,8-diazabicyclo[5,4,0]undec-7-ene) (19.72 mL) in tetrahydrofuran (50 mL) was added dropwise. The stirring mixture became cloudy and was cooled in an ice-water bath to an internal temperature of 15° C. A mixture of Example 16A (32 g) in tetrahydrofuran (50 mL) was added over 30 minutes. The reaction was stirred overnight, cooled to an internal temperature of 5° C., and quenched by the addition of 1% by weight aqueous citric acid (700 mL). Ethyl acetate (400 mL) was added and the layers were separated. The organic layer was washed with saturated aqueous sodium chloride solution (400 mL), dried over anhydrous magnesium sulfate, filtered and concentrated under reduced pressure. The crude material was purified by flash column chromatography on a Grace Reveleris system using a Teledyne Isco RediSep® Gold 330 g column, eluting with a 0-25% ethyl acetate/heptanes gradient to provide the title compound in a 9:1 mixture of E- and Z-isomers. E-isomer: ¹H NMR (501 MHz, Chloroform-d) δ ppm 7.39 (ddt, 2H), 7.36 (ddd, 2H), 7.32-7.27 (m, 1H), 6.88 (dd, 1H), 6.85 (d, 1H), 6.76 (d, 1H), 6.71 (ddd, 1H), 5.01 (s, 2H), 2.22 (s, 3H), 1.34 (s, 9H), 0.97 (s, 9H), 0.17 (s, 6H). MS (ESI) m/z 515.9 (M+NH₄)⁺. This isomer was assigned E by 2D NOE experiments. Z-isomer: ¹H NMR (501 MHz, Chloroform-d) δ ppm 7.74 (s, 1H), 7.45 (ddt, 2H), 7.38 (ddd, 2H), 7.35-7.30 (m, 1H), 7.29-7.26 (m, 1H), 6.83 (d, 1H), 6.79 (dd, 1H), 5.06 (s, 2H), 2.30 (d, 3H), 1.53 (s, 9H), 0.99 (s, 9H), 0.18 (s, 6H). MS (ESI) m/z 515.9 (M+NH₄)⁺. This isomer was assigned Z by 2D NMR experiments.

### Example 16D

### (R)-tert-butyl 2-acetoxy-3-(2-(benzyloxy)-5-((tert-butyldimethylsilyl)oxy)phenyl)propanoate

A 600 mL stainless steel reactor was charged with (1,2-bis[(2R,5R)-2,5-diethylphospholano]benzene(1,5-cyclooctadiene)rhodium (I) trifluoromethanesulfonate (1.88 g), followed by a solution of Example 16C (34.86 g) in methanol (350 mL). The reactor was purged with nitrogen 3 times and 2 times with hydrogen. The mixture was stirred at 1200 RPM under 120 psi of hydrogen with no external heating for 24 hours. The mixture was concentrated under reduced pressure, suspended in 5:1 heptanes/dichloromethane (70 mL) and filtered through a pad of diatomaceous earth. The filtrate was concentrated under reduced pressure and purified on a Grace Reveleris system using a 750 g Teledyne Isco Redisep® gold column eluting with an ethyl acetate/heptanes gradient (0-25%). The desired fractions were concentrated under reduced pressure to provide the title compound. ¹H NMR (500 MHz, Chloroform-d) δ ppm 7.45 (d, 2H), 7.42-7.34 (m, 2H), 7.34-7.28 (m, 1H), 6.77 (d, 1H), 6.70 (d, 1H), 6.67 (dd, 1H), 5.19 (dd, 1H), 5.05 (d, 1H), 5.01 (d, 1H), 3.29 (dd, 1H), 2.92 (dd, 1H), 2.03 (s, 3H), 1.40 (s, 9H), 0.97 (s, 9H), 0.16 (s, 6H). MS (DCI) m/z 518.2 (M+NH₄)⁺.

### Example 16E

### (R)-tert-butyl 3-(2-(benzyloxy)-5-((tert-butyldimethylsilyl)oxy)phenyl)-2-hydroxypropanoate

An oven dried 250 mL 3-neck flask was charged with Example 16D (27.46 g). The flask was equipped with a magnetic star bar and rubber septa, and vacuum purged with nitrogen gas twice. Anhydrous ethanol (274 mL) was added, and the mixture was stirred. To the stirring solution was added dropwise sodium ethoxide (21% wt in ethanol, 1.024 mL). The reaction was stirred for three hours at ambient temperature and was quenched by addition of acetic acid (0.3 mL). The bulk of the solvents were removed by rotary evaporation, and the material was diluted with ethyl acetate (300 mL). Saturated aqueous sodium bicarbonate was added (300 mL). The layers were separated and the aqueous layer was extracted with ethyl acetate (300 mL). The combined organic layers were washed with saturated aqueous sodium chloride, dried over MgSO₄, treated with activated charcoal (0.5 g) and stirred for 1 hour before filtering through diatomaceous earth to give the title compound after concentration under reduced pressure. ¹H NMR (400 MHz, chloroform-d) δ ppm 7.48-7.42 (m, 2H), 7.42-7.36 (m, 2H), 7.36-7.29 (m, 1H), 6.79 (d, 1H), 6.75 (d, 1H), 6.67 (dd, 1H), 5.10-4.99 (m, 2fH), 4.39 (ddd, 1H), 3.16 (dd, 1H), 2.91 (d, 1H), 2.86 (dd, 1H), 1.41 (s, 9H), 0.99 (s, 9H), 0.18 (s, 6H). MS (DCI) m/z 476.2 (M+NH₄)⁺.

### Example 16F

### tert-butyl (R)-2-((5-(4-(((R)-1-(allyloxy)-3-(bis(4-methoxyphenyl) (phenyl)methoxy) propan-2-yl)oxy)-3,5-dichloro-2,6-dimethylphenyl)-6-(4-fluorophenyl)thieno[2,3-d]pyrimidin-4-yl)oxy)-3-(2-(benzyloxy)-5-((tert-butyldimethylsilyl)oxy)phenyl)propanoate

Example 15K (14.7 g). Example 16E (8.52 g), and cesium carbonate (11.01 g) were added to a three-necked flask equipped with an overhead stirrer and 2.2 g of 4 mm glass beads, tert-Butanol (145 mL) was added and the mixture was heated to 65° C. for 3 hours. Additional cesium carbonate (5.50 g) was added the reaction was stirred at 65° C. overnight. The reaction mixture was cooled and was diluted with ethyl acetate (300 mL). The resulting solution was filtered through diatomaceous earth, and washed through with 200 mL ethyl acetate. The mixture was concentrated, taken up in toluene and purified by silica gel chromatography using 10-30% ethyl acetate in heptanes as the eluent to provide the title compound. MS (ESI) m/z 1293.3 (M+H)⁺.

### Example 16G

### tert-butyl (R)-2-((5-(4-(((S)-1-(allyloxy)-3-hydroxypropan-2-yl)oxy)-3,5-dichloro-2,6-dimethylphenyl)-6-(4-fluorophenyl)thieno[2,3-d]pyrimidin-4-yl)oxy)-3-(2-(benzyloxy)-5-((tert-butyldimethylsilyl)oxy)phenyl)propanoate

Example 16F (17.11 g) in dichloromethane (65 mL) and methanol (65 mL) was cooled to 0° C. Formic acid (38 mL) was added and the solution was stirred for 15 minutes at 0° C. The mixture was slowly added to 1 L of vigorously stirred saturated aqueous sodium bicarbonate. The resulting mixture was extracted with ethyl acetate (2×500 mL). The combined organics were washed with brine (100 mL), dried over Na₂SO₄, filtered, and concentrated. The crude material was purified by silica gel chromatography using 10-30% ethyl acetate in heptanes as eluent to provide the title compound. MS (ESI) m/z 988.9 (M+H)⁺.

### Example 16H

### (R)-tert-butyl 2-((5-(4-(((R)-1-(allyloxy)-3-(tosyloxy) propan-2-yl)oxy)-3,5-dichloro-2,6-dimethylphenyl)-6-(4-fluorophenyl)thieno[2,3-d]pyrimidin-4-yl)oxy)-3-(2-(benzyloxy)-5-((tert-butyldimethylsilyl)oxy)phenyl)propanoate

Example 16G (13.04 g) was dissolved in dichloromethane (125 mL) and the mixture was cooled to 0° C. para-Toluenesulfonyl chloride (3.77 g), and 1,4-diazabicyclo[2.2.2]octane (2.95 g) were added, and the reaction was stirred at 0° C. for 30 minutes. The mixture was diluted with 55 mL dichloromethane, and quenched with 55 mL saturated aqueous NH₄Cl. The layers were separated and the organic layer was washed with brine, dried over Na₂SO₄, filtered, and concentrated. The crude material was purified by silica gel chromatography using 10-25% ethyl acetate in heptanes to provide the title compound. MS (ESI) m/z 1145.1 (M+H)⁺.

### Example 161

### (R)-tert-butyl 2-((5-(4-(((R)-1-(allyloxy)-3-(tosyloxy) propan-2-yl)oxy)-3,5-dichloro-2,6-dimethylphenyl)-6-(4-fluorophenyl)thieno[2,3-d]pyrimidin-4-yl)oxy)-3-(2-(benzyloxy)-5-hydroxyphenyl)propanoate

To Example 16H (14.15 g) in tetrahydrofuran (120 mL) was added acetic acid (0.779 mL), and tetrabutylammonium fluoride (13.60 mL, 1 M in tetrahydrofuran). The reaction mixture was stirred for 20 minutes. The mixture was quenched with 20 mL saturated aqueous sodium bicarbonate solution. The mixture was diluted with 20% ethyl acetate/heptanes (150 mL). The layers were separated and the organic layer was washed with water and brine, dried over Na₂SO₄, filtered, and concentrated. The crude material was purified by silica gel chromatography using 10-50% ethyl acetate in heptanes to provide the title compound. ¹H NMR (400 MHz, dimethylsulfoxide-d₆) δ ppm 8.90 (s, 1H), 8.64 (s, 1H), 7.70 (d, 2H), 7.40 (d, 2H), 7.30 (m, 7H), 7.21 (m, 2H), 7.05 (t, 1H), 6.81 (d, 1H), 6.57 (m, 1H), 6.17 (d, 1H), 5.65 (m, 1H), 5.20 (t, 1H), 5.00 (m, 2H), 4.50 (m, 1H), 4.25 (m, 2H), 3.72 (m, 2H), 3.56 (m, 2H), 2.66 (m, 1H), 2.39 (s, 3H), 2.14 (s, 3H), 1.82 (s, 3H), 1.21 (s, 9H). MS (ESI) m/z 1030.7 (M+H)⁺.

### Example 16J

### tert-butyl (7R,16R)-10-(benzyloxy)-19,23-dichloro-1-(4-fluorophenyl)-20,22-dimethyl-16-{[(prop-2-en-1-yl)oxy]methyl}-7,8,15,16-tetrahydro-18,21-etheno-13,9-(metheno)-6,14,17-trioxa-2-thia-3,5-diazacyclononadeca[1,2,3-cd]indene-7-carboxylate

To Example 161 (11.88 g) in N,N-dimethylformamide (1160 mL) was added cesium carbonate (18.79 g) and the reaction was stirred for 2 hours. The solution was poured into water (3600 mL), and the aqueous solution was extracted with ethyl acetate (4×300 mL). The combined organics were washed with water (2×800 mL) and brine (500 mL), dried over Na₂SO₄, filtered, and concentrated.

The crude material was purified by silica gel chromatography using 10-50% ethyl acetate in heptanes to provide the title compound. ¹H NMR (500 MHz, dimethylsulfoxide-d₆) δ ppm 8.75 (s, 1H), 7.40 (m, 5H), 7.20 (m, 4H), 6.90 (m, 2H), 5.98 (m, 1H), 5.92 (m, 1H), 5.68 (s, 1H), 5.30 (d, 1H), 5.19 (d, 1H), 5.02 (q, 2H), 4.81 (m, 1H), 4.51 (dd, 1H), 4.36 (d, 1H), 4.03 (m, 2H), 3.75 (m, 2H), 3.58 (m, 1H), 2.81 (m, 1H), 2.05 (s, 3H), 1.91 (s, 3H), 1.09 (s, 9H). MS (ESI) m/z 857.0 (M+H)⁺.

### Example 16K

### tert-butyl (7R,16R)-10-(benzyloxy)-19,23-dichloro-1-(4-fluorophenyl)-16-(hydroxymethyl)-20,22-dimethyl-7,8,15,16-tetrahydro-18,21-etheno-13,9-(metheno)-6,14,17-trioxa-2-thia-3,5-diazacyclononadeca[1,2,3-cd]indene-7-carboxylate

A solution of Example 16J (8.75 g) in tetrahydrofuran (120 mL) and methanol (80 mL) was degassed and flushed with nitrogen three times. Tetrakis(triphenylphosphine)palladium (0) (1.179 g), and 1,3-dimethylpyrimidine-2,4,6(1H,3H,5H)-trione (3.98 g) were added, and the solution was degassed and flushed with nitrogen once. The reaction mixture was stirred overnight. Pyrrolidine-1-carbodithioic acid, ammonia salt (0.251 g) was added as a palladium scavenger, and the reaction was stirred for 30 minutes. Ethyl acetate (100 mL) was added and the mixture was filtered through diatomaceous earth, washing with more ethyl acetate. The crude material was concentrated and used without further purification. MS (ESI) m/z 819.2 (M+H)⁺.

### Example 16L

### tert-butyl (7R,16S)-10-(benzyloxy)-19,23-dichloro-1-(4-fluorophenyl)-20,22-dimethyl-16-{[(4-methylbenzene-1-sulfonyl)oxy]methyl}-7,8,15,16-tetrahydro-18,21-etheno-13,9-(metheno)-6,14,17-trioxa-2-thia-3,5-diazacyclononadeca[1,2,3-cd]indene-7-carboxylate

Example 16K (8.09 g) in dichloromethane (95 mL) was cooled to 0° C. To the mixture was added p-toluenesulfonyl chloride (4.9 g), and 1,4-diazabicyclo[2.2.2]octane (3.9 g). The reaction was stirred at 0° C. for 1 hour. The mixture was diluted with 50 mL dichloromethane, and quenched with 50 mL saturated aqueous NH₄Cl. Water (50 mL) was added and the layers were separated. The organic layer was washed with brine, dried over Na₂SO₄, filtered, and concentrated. The crude material was purified by silica gel chromatography using 10-35% ethyl acetate in heptanes to provide the title compound. MS (ESI) m/z 971.2 (M+H)⁺.

### Example 16M

### tert-butyl (7R,16R)-10-(benzyloxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To an ambient solution of Example 16L (2.98 g) in N,N-dimethylformamide (10 mL) was added 1-methylpiperazine (10.20 mL). The reaction was heated to 40° C. for 24 hours. Additional 1-methyl-piperazine (2 mL) was added and the reaction was heated at 35° C. overnight. The reaction was cooled to room temperature, and the solvents were removed by rotary evaporation. The crude material was cooled in an ice bath, stirred, and diluted sequentially with ethyl acetate (100 mL) and water (100 mL). The layers were separated, and the aqueous layer was extracted with additional ethyl acetate (2×100 mL). The combined organics were washed with brine (2×100 mL), dried over anhydrous sodium sulfate, filtered and concentrated under reduced pressure. The residue was diluted with toluene (5 mL) and was purified by normal phase MPLC (Biotage® Isolera, 100 g Biotage® Ultra SiO₂ column), eluting with a gradient of 0-6% methanol in dichloromethane to provide the title compound. ¹H NMR (500 MHz, dimethylsulfoxide-d₆) δ ppm 8.74 (s, 1H), 7.41 (m, 2H), 7.39 (m, 2H), 7.35 (m, 1H), 7.20 (m, 4H), 6.90 (m, 1H), 6.81 (m, 1H), 6.00 (m, 1H), 5.67 (s, 1H), 5.02 (q, 2H), 4.75 (m, 1H), 4.44 (m, 2H), 3.60 (m, 1H), 3.58 (m, 1H), 2.80 (m, 1H), 2.48 (m, 3H), 2.40 (m, 4H), 2.30 (m, 4H), 2.15 (s, 3H), 2.08 (s, 3H), 1.89 (s, 3H), 1.09 (s, 9H). MS (ESI) m/z 899.4 (M+H)⁺.

### Example 16N

### tert-butyl (7R,16R)-19,23-dichloro-1-(4-fluorophenyl)-10-hydroxy-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 16M (1.943 g) in tetrahydrofuran (11 mL) was added to 5% Pd/C (1.801 g) in a 20 mL Barnstead Hast C pressure reactor. The reactor was purged with argon gas. The mixture was stirred at 1600 rpm under 50 psi of hydrogen at 25° C. After 17.3 hours, the reaction was vented. The mixture was filtered through a filter funnel with a polyethylene frit packed with diatomaceous earth. The mixture was concentrated, and the crude material was taken up in ether and a small amount of dichloromethane. The mixture was filtered through diatomaceous earth, washing with ether/dichloromethane. The solvent was removed on a rotovap, and the residue was placed on high vacuum overnight. ¹H NMR (500 MHz, dimethylsulfoxide-d₆) δ ppm 9.11 (s, 1H), 8.72 (s, 1H), 7.20 (m, 4H), 6.67 (m, 2H), 5.96 (m, 1H), 5.50 (s, 1H), 4.69 (m, 1H), 4.41 (m, 1H), 4.37 (m, 1H), 3.54 (dd, 1H), 3.58 (m, 1H), 2.62 (m, 2H), 2.22-2.50 (m, 9H), 2.18 (s, 6H), 1.88 (s, 3H), 1.09 (s, 9H). MS (ESI) m/z 811.2 (M+H)⁺.

### Example 160

### tert-butyl (7R,16R)-19,23-dichloro-1-(4-fluorophenyl)-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (75 mg), Example 13C (56 mg), triphenylphosphine (74 mg) and di-tert-butylazodicarboxylate (48 mg). The vial was capped with a septum, then evacuated and backfilled with nitrogen gas. Toluene (0.46 mL) and tetrahydrofuran (0.46 mL) were added, and the vial was evacuated and backfilled with nitrogen gas again. The reaction mixture was heated to 50° C. for one hour. The mixture was concentrated and purification by flash chromatography on an AnaLogix IntelliFlash²⁸⁰ system (10 g silica gel cartridge (eluting with 0-8% methanol/dichloromethane) provided the title compound. MS (ESI) m/z 1239.4 (M+H)⁺

### Example 16P

### (7R,16R)-19,23-dichloro-1-(4-fluorophenyl)-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 160 (100 mg) in dichloromethane (0.7 mL) was added trifluoroacetic acid (TFA) (0.700 mL). The mixture was stirred for 4 hours, concentrated in vacuo, and dissolved in acetonitrile. The solution was made basic with saturated aqueous NaHCO₃, and was filtered. The filtrate was purified by reverse phase preparative LC using a Gilson 2020 system (Luna™ C-18, 250×50 mm column, mobile phase A: 0.1% ammonium acetate in water; B: acetonitrile: 5-100% B to A gradient at 70 mL/minute) to provide the title compound. ¹H NMR (400 MHz, dimethylsulfoxide-d₆) δ ppm 8.81 (d, 1H), 8.74 (s, 1H), 8.36-8.31 (m, 2H), 7.43 (d, 1H), 7.23-7.10 (m, 5H), 7.10-7.03 (m, 2H), 6.88 (d, 1H), 6.75 (dd, 1H), 6.25 (dd, 1H), 5.81 (d, 1H), 5.24 (d, 1H), 5.16 (d, 1H), 4.85 (p, 1H), 4.44 (d, 2H), 4.17 (dd, 2H), 3.77 (dd, 2H), 3.69-3.58 (m, 3H), 3.56-3.50 (m, 4H), 3.44-3.42 (m, 2H), 3.23 (s, 3H), 3.03-2.93 (m, 1H), 2.66 (td, 2H), 2.42 (s, 8H), 2.20 (s, 3H), 1.99 (s, 3H), 1.95 (s, 3H). MS (ESI) m/z 1083.3 (M+H)⁺.

### Example 17

### (7R,16R,21S)-10-({2-[4-(2-carboxyethyl)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 17A

### 3-(4-(4-(hydroxymethyl)pyrimidin-2-yl)phenyl)propanoic Acid

To a solution of (2-chloropyrimidin-4-yl)methanol (500 mg) and 3-(4-boronophenyl)propanoic acid (671 mg) in a solvent mixture of tetrahydrofuran (14.7 mL) and saturated aqueous sodium bicarbonate solution (8.40 mL) was added palladium (0) tetrakis(triphenylphosphine) (400 mg). The reaction was heated to 75° C. overnight. The reaction was cooled to room temperature and diluted with 15% sodium hydroxide solution (30 mL) and diethyl ether (30 mL). The layers were separated, and the organic layer was discarded. The aqueous layer was acidified with concentrated hydrochloric acid to a pH of ˜5. The aqueous layer was extracted with dichloromethane (3×100 mL). The combined organics were dried with anhydrous sodium sulfate, filtered and concentrated under reduced pressure. The material was used in the subsequent step without further purification. MS (ESI) m/z 259.1 (M+H)⁺.

### Example 17B

### tert-butyl 3-(4-(4-(hydroxymethyl)pyrimidin-2-yl)phenyl)propanoate

To an ambient solution of Example 17A (600 mg) in a solvent mixture of dichloromethane (5.8 mL) and tert-butanol (5.8 mL) was added solid ammonia hydrochloride (373 mg). The mixture was cooled to 0° C. and (E)-tert-butyl N,N′-diisopropylcarbamimidate (1396 mg.) was added via syringe. The reaction mixture was removed from the ice bath and stirred overnight. Additional ammonia hydrochloride (373 mg) and (E)-tert-butyl N,N′-diisopropylcarbamimidate (1396 mg) were added every 4 hours until the reaction was complete. A total of 1.49 g of ammonium chloride and 5.58 g of (E)-tert-butyl N,N′-diisopropylcarbamimidate were added to the reaction. The reaction was diluted with saturated aqueous ammonium chloride solution (50 mL) and methyl tert-butyl ether (50 mL), and the mixture was stirred vigorously for 1 hour. The mixture was filtered through a sintered glass funnel to remove the solid. The layers of the filtrate were separated, and the aqueous layer was extracted with additional methyl tert-butyl ether (2×50 mL). The combined organics were dried with anhydrous sodium sulfate, filtered and concentrated under reduced pressure. The residue was dissolved in 6 mL of toluene, and the solution was purified by silica gel chromatography (Biotage® Isolera, 50 g silica gel column), eluting with a gradient of 0-50% ethyl acetate in heptane, to give the title compound. MS (ESI) m/z 315.3 (M+H)⁺.

### Example 17C

### tert-butyl (7R,16R)-10-({2-[4-(3-tert-butoxy-3-oxopropyl)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a cold (0° C.) solution of Example 17B (49.6 mg), Example 12P (40 mg) and triphenylphosphine (41.3 mg) in toluene was added (E)-di-tert-butyl diazene-1,2-dicarboxylate (36.3 mg). The cold bath was removed, and the reaction was stirred overnight. The mixture was directly purified by silica gel chromatography (Biotage® Isolera, 10 g silica gel column), eluting with a gradient of 0-6% methanol in dichloromethane, to give the title compound. MS (ESI) m/z 1057.5 (M+H)⁺.

### Example 17D

### (7R,16R,21S)-10-({2-[4-(2-carboxyethyl)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 17C (36 mg) in dichloromethane (0.5 mL) was added trifluoroacetic acid (0.262 mL), and the reaction was stirred overnight. The reaction was concentrated under reduced pressure. The residue was dissolved in 2:1 dimethylsulfoxide:water (3 mL) and purified by reverse phase HPLC (Phenomenex® Luna™ 250×50 mm column) eluting with a gradient of 5 to 85% acetonitrile in water containing 0.1% v/v trifluoroacetic acid. The fractions containing the product were lyophilized to give the title compound. ¹H NMR (400 MHz, dimethylsulfoxide-d₆) δ ppm 12.15 (s, 1H), 9.44 (s, 1H), 8.82 (d, 1H), 8.67 (s, 1H), 8.25 (d, 2H), 7.44 (d, 1H), 7.34 (d, 2H), 7.21-7.02 (m, 6H), 6.87 (dd, 2H), 6.77 (dd, 1H), 6.10 (dd, 1H), 5.60 (d, 1H), 5.27-5.05 (m, 2H), 4.52 (q, 1H), 4.41 (d, 1H), 4.30 (dd, 1H), 3.85 (d, 1H), 3.35-2.96 (m, 7H), 2.91-2.79 (m, 3H), 2.78-2.61 (m, 5H), 2.55 (t, 2H), 2.16 (s, 3H). MS (ESI) m/z 945.6 (M+H)⁺.

### Example 18

### (7R,16R,21S)-19-chloro-10-[(2-{4-[(2R)-2,3-dihydroxypropoxy]phenyl}pyrimidin-4-yl)methoxy]-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 18A

### methyl 2-(4-((tert-butyldimethylsilyl)oxy)phenyl)pyrimidine-4-carboxylate

In a 5 mL microwave vial, a mixture of methyl 2-chloropyrimidine-4-carboxylate (250 mg) and (4-((tert-butyldimethylsilyl)oxy)phenyl)boronic acid (384 mg) were suspended in previously degassed 1,4-dioxane (2.5 mL). Potassium carbonate (250 mg) was solubilized in previously degassed water (0.5 mL) and added onto the reaction mixture. [1,1′-Bis(diphenylphosphino)ferrocene]dichloropalladium (II) (35 mg) was added and the reaction mixture was placed under an argon atmosphere and heated under microwave at 130° C. for 45 minutes. Methyl 2-chloropyrimidine-4-carboxylate (125 mg) and [1,1′-bis(diphenylphosphino)ferrocene]dichloropalladium (II) (35 mg) were added again and the reaction mixture was further heated at 130° C. for 30 minutes. The reaction mixture was diluted with 50 mL of dichloromethane and 30 mL of water and the aqueous layer was extracted with 3×50 mL of dichloromethane. The organic layer was dried over magnesium sulfate, filtered, and concentrated. The crude product was purified by silica gel flash chromatography eluting with 5-10% ethyl acetate in cyclohexane to afford the title compound. ¹H NMR (300 MHz, CDCl₃): δ ppm 8.97 (d, 1H), 8.41 (d, 2H), 7.78 (d, 1H), 6.95 (d, 2H), 4.04 (s, 3H), 1.01 (s, 9H), 0.24 (s, 6H). MS (ESI) m/z 344.9 (M+H)⁺.

### Example 18B

### (2-(4-((tert-butyldimethylsilyl)oxy)phenyl)pyrimidin-4-yl)methanol

To a solution of Example 18A (1.27 g) in tetrahydrofuran (10 mL) and methanol (20 mL) was added at 0° C., sodium borohydride (0.488 g). The reaction mixture was stirred at 0° C. for 3 hours. The reaction mixture is quenched with 40 mL of saturated aqueous ammonium chloride solution. The organic solvents were removed under reduced pressure and the residue was diluted with 100 mL of dichloromethane and 50 mL of water. The aqueous layer was extracted with 3×50 mL of dichloromethane. The organic layer was washed with brine, dried over magnesium sulfate, filtered, and concentrated. The crude product was purified by silica gel flash chromatography eluting with 0-20% ethyl acetate in cyclohexane to afford the title compound. ¹H NMR (300 MHz, CDCl₃) δ ppm 8.70 (d, 1H), 8.36 (d, 2H), 7.08 (d, 1H), 6.94 (d, 2H), 4.78 (d, 2H), 3.67 (t, 1H), 1.00 (s, 9H), 0.24 (s, 6H). MS (ESI) m/z 317.0 (M+H)⁺.

### Example 18C

### 4-(4-(hydroxymethyl)pyrimidin-2-yl)phenol

To a solution of Example 18B (200 mg) in methanol (5 mL) was added cesium fluoride (144 mg). The mixture was stirred at ambient temperature for 1 hour. The mixture was diluted with ethyl acetate and washed with water. The organic layer was dried over sodium sulfate, filtered, and concentrated. The crude product was purified by silica gel flash chromatography, eluting with 30-80% ethyl acetate in hexanes to afford the title compound. LC/MS (ESI) m/z 203.07 (M+H)⁺.

### Example 18D

### (S)-(2-(4-((2,2-dimethyl-1,3-dioxolan-4-yl)methoxy)phenyl)pyrimidin-4-yl)methanol

To a solution of Example 18C (238 mg) in dimethylformamide (3.5 mL) was added (R)-(2,2-dimethyl-1,3-dioxolan-4-yl)methyl 4-methylbenzenesulfonate (371 mg) and cesium carbonate (460 mg). The mixture was stirred at 50° C. for 1 day. The mixture was diluted with ethyl acetate and washed with water. The organic layer was dried over sodium sulfate, filtered, and concentrated. The residue was purified by silica gel flash chromatography, eluting with 30-80% ethyl acetate in hexanes to give the title compound. MS (ESI) m/z 317.1 (M+H)⁺.

### Example 18E

### tert-butyl (7R,16R)-19-chloro-10-{[2-(4-{[(4S)-2,2-dimethyl-1,3-dioxolan-4-yl]methoxy}phenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 12P (60 mg), Example 18D (49.9 mg) and triphenylphosphine (43.4 mg). The vial was capped with a septum then evacuated and backfilled with nitrogen twice. Toluene (0.79 mL) was added and once all the reagents completely dissolved, the mixture was cooled with an ice bath. Di-tert-butyl azodicarboxylate (36.3 mg) was added in one solid portion. The vial was capped with a septum, evacuated and backfilled with nitrogen twice again. The mixture was stirred at 0° C. for 10 minutes, the cooling bath was removed, and the mixture was allowed to stir overnight. The mixture was concentrated and purified by silica gel flash chromatography eluting with 0-8% methanol in dichloromethane to afford the title compound. MS (ESI) m/z 1059.8 (M+H)⁺.

### Example 18F

### (7R,16R,21S)-19-chloro-10-[(2-{4-[(2R)-2,3-dihydroxypropoxy]phenyl}pyrimidin-4-yl)methoxy]-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 18E (26 mg) in dichloromethane (0.2 mL) was added trifluoroacetic acid (0.20 mL). The mixture was stirred for 4 hours and concentrated in vacuo. The residue was dissolved in acetonitrile, basified with saturated aqueous sodium bicarbonate and filtered to remove solids. The filtrate was purified by reverse phase prep LC using a Gilson 2020 system (Luna™, C-18, 250×50 mm column, Mobile phase A: 0.1% trifluoroacetic acid in water; B: acetonitrile; 20-75% B to A gradient at 70 mL/minute) to afford the title compound after lyophilization. ¹H NMR (400 MHz, dimethylsulfoxide-d₆) δ ppm 8.81 (d, 1H), 8.70 (s, 1H), 8.39-8.26 (m, 2H), 7.41 (d, 1H), 7.23-7.02 (m, 7H), 6.90 (dd, 2H), 6.80 (dd, 1H), 6.13 (dd, 1H), 5.64 (d, 1H), 5.30-5.03 (m, 2H), 4.58 (q, 1H), 4.46-4.28 (m, 2H), 4.18-2.98 (m, 14H), 2.99-2.81 (m, 2H), 2.80-2.65 (m, 6H), 2.19 (s, 3H). MS (ESI) m/z 963.4 (M+H)⁺.

### Example 19

### (7R,16R)-10-{[2-(2-carboxyphenyl)pyrimidin-4-yl]methoxy}-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 19A

### tert-butyl 2-(4-(hydroxymethyl)pyrimidin-2-yl)benzoate

tert-Butyl 2-(4,4,5,5-tetramethyl-1,3,2-dioxaborolan-2-yl)benzoate (250 mg) and (2-bromopyrimidin-4-yl)methanol (179 mg) were dissolved in 1,4-dioxane (3.5 mL). Aqueous sodium carbonate (2 M, 1.23 mL) was added. The mixture was degassed and flushed with nitrogen three times. Dichloro[1,1′-bis(diphenylphosphino)ferrocene]palladium (II) dichloromethane adduct (67.1 mg) was added, and the mixture was degassed and flushed with nitrogen once. The mixture was stirred at 75° C. overnight. The mixture was cooled, diluted with ethyl acetate (10 mL), washed with saturated aqueous sodium bicarbonate (10 mL), washed with brine (10 mL), and dried over anhydrous sodium sulfate. After filtration, the mixture was concentrated and the residue was purified by flash column chromatography on silica gel using a 20-70% gradient of ethyl acetate in heptanes to provide the title compound. ¹H NMR (500 MHz, dimethylsulfoxide-d₆) δ ppm 8.97 (d, 1H), 7.80 (dd, 1H), 7.66-7.54 (m, 4H), 5.72 (t, 1H), 4.60 (d, 2H), 1.31 (s, 9H). MS (ESI) m/z 213.1 (M-tBu-water+H)⁺.

### Example 19B

### (7R,16R)-10-{[2-(2-carboxyphenyl)pyrimidin-4-yl]methoxy}-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 12P (60 mg), Example 19A (45.1 mg) and triphenylphosphine (43.4 mg) were stirred in toluene (0.8 mL) at 0° C. until everything dissolved. (E)-di-tert-butyl diazene-1,2-dicarboxylate (36.3 mg) was added and the solution was stirred at room temperature overnight. The crude material was chromatographed on silica gel using 0-10% methanol in dichloromethane to give the coupled ester. The material was taken up in dichloromethane (0.2 mL) and trifluoroacetic acid (0.3 mL), and the solution was stirred for six hours and concentrated. The crude material was taken up in N,N-dimethylformamide (1 mL) and water (1 mL), and purified by reverse phase chromatography using a 30-100% gradient of acetonitrile in water (with 0.1% trifluoroacetic acid) over 40 minutes on a Grace Reveleris equipped with a Luna™ column: C18(2), 100 Å, 250×50 mm. The fractions containing the desired compound were combined, frozen and lyophilized to isolate the title compound. ¹H NMR (500 MHz, dimethylsulfoxide-d₆) δ ppm 8.95 (d, 1H), 8.81 (s, 1H), 7.78 (dd, 1H), 7.75 (d, 1H), 7.66 (m, 2H), 7.60 (d, 1H), 7.29-7.26 (m, 4H), 7.22 (d, 1H), 7.03 (d, 1H), 6.96 (d, 1H), 6.85 (dd, 1H), 6.23 (m, 1H), 5.78 (d, 1H), 5.24 (q, 2H), 4.63 (m, 1H), 4.58 (d, 1H), 4.40 (dd, 1H), 4.04 (dd, 1H), 2.92 (d, 2H), 2.74-2.62 (m, 3H), 2.58-2.45 (m, 6H), 2.33 (s, 3H), 2.30 (s, 3H). MS (ESI) m/z 917.3 (M+H)⁺.

### Example 20

### (7R,16R)-10-({2-[4-(2-carboxyethyl)phenyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 20A

### tert-butyl (7R,16R)-10-({2-[4-(3-tert-butoxy-3-oxopropyl)phenyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 20A was prepared according to the procedure described for Example 17C, substituting Example 16N for Example 12P. MS (ESI) m/z 1105.6 (M+H)⁺.

### Example 20B

### (7R,16R)-10-({2-[4-(2-carboxyethyl)phenyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 20B was prepared according to the procedure described for Example 17D, substituting Example 20A for Example 17C. ¹H NMR (400 MHz, dimethylsulfoxide-d₆) δ ppm 12.18 (s, 1H), 9.41 (s, 1H), 8.88 (d, 1H), 8.76 (s, 1H), 8.35-8.28 (m, 2H), 7.50 (d, 1H), 7.38 (d, 2H), 7.24-7.11 (m, 5H), 6.91 (d, 1H), 6.82 (dd, 1H), 6.28 (dd, 1H), 5.79 (d, 1H), 5.23 (q, 2H), 4.92 (q, 1H), 4.54-4.39 (m, 2H), 3.65 (dd, 1H), 3.20 (d, 2H), 3.14-2.86 (m, 7H), 2.83 (t, 1H), 2.79 (s, 3H), 2.59 (t, 2H), 2.45 (s, 2H), 1.99 (s, 3H), 1.96 (s, 3H). MS (ESI) m/z 993.3 (M+H)⁺.

### Example 21

### (7R,16R,21S)-19-chloro-1-(4-fluorophenyl)-16-[(4-{2-[2-(2-methoxyethoxy)ethoxy]ethyl}piperidin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 21A

### 2-(2-methoxyethoxy)ethyl 4-methylbenzenesulfonate

1,4-Diazabicyclo[2.2.2]octane (3.73 g) was added to a solution of 2-(2-methoxyethoxy)ethanol (2.00 g) and p-toluenesulfonyl chloride (4.76 g) in dichloromethane (30 mL). After stirring for 1 hour at room temperature, water (10 mL) was added and the mixture was stirred for 10 minutes. Separation via Horizon DryDisk®, removal of the solvent in vacuo, followed by purification by chromatography on silica gel using a CombiFlash® system (24 g RediSep® Gold column, eluting with 0-12% cyclohexane/ethyl acetate) provided the title compound which was used in the next step without further purification. MS (APCI) m/z 275.2 (M+H)⁺.

### Example 21B

### 1-benzyl-4-(2-(2-(2-methoxyethoxy)ethoxy)ethyl)piperidine

A solution of Example 21A (500 mg) in dry dimethylformamide (5 mL) was added to a mixture of 2-(1-benzylpiperidin-4-yl)ethanol (360 mg) and NaH (98 mg, 60%) in dry dimethylformamide (14 mL). The suspension obtained was stirred for 4 hours at 60° C., and overnight at room temperature, followed by additional 8 hours at 60° C. Water was added (10 mL). The mixture was extracted with ethyl acetate, and washed with water, saturated aqueous NaHCO₃ solution, and brine. The organic layer was dried over magnesium sulfate, filtered, and concentrated. The crude product was purified by chromatography on silica gel using a Grace Reveleris system (12 g Grace Reveleris column, eluting with 1-20% dichloromethane/methanol) to provide the title compound. MS (APCI) m/z 322.2 (M+H)⁺.

### Example 21C

### 4-(2-(2-(2-methoxyethoxy)ethoxy)ethyl)piperidine

A solution of Example 21B (137 mg) in methanol (8 mL) was subjected to hydrogenation in an H-cube (ThalesNano, CatCart Pd/C 10%, flow rate 1 mL/minute, 70° C.). Removal of the solvent provided the crude title compound which was used in the next step without further purification.

### Example 21D

### ethyl (7R,16R,21S)-19-chloro-1-(4-fluorophenyl)-16-[(4-{2-[2-(2-methoxyethoxy)ethoxy]ethyl}piperidin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

Triethylamine (60.7 mg) was added to a mixture of Example 4N (75.0 mg) and Example 21C (52.0 mg) in N,N-dimethylformamide (2 mL). After heating in the Q-tube for 3 days at 45° C. more triethlyamine (0.05 mL) was added and the stirring was continued for 2 days at 45° C. Water (5 mL) was added. The mixture was extracted with ethyl acetate, and the combined organic layers were washed with water, brine and dried (MgSO₄). After filtration and concentration, the crude product was purified by chromatography on silica gel using a CombiFlash® system (4 g RediSep® Gold column, eluting with 0-100% cyclohexane/ethyl acetate) to provide the title compound. MS (APCI) m/z 1062.4 (M+H)⁺.

### Example 21E

### (7R,16R,21S)-19-chloro-1-(4-fluorophenyl)-16-[(4-{2-[2-(2-methoxyethoxy)ethoxy]ethyl}piperidin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic Acid

A solution of LiOH (22.8 mg) in water (1 mL) was added to a solution of Example 21D (39.0 mg) in a mixture of ethanol (1 mL) and tetrahydrofuran (1 mL). After stirring overnight at room temperature, trifluoroacetic acid (0.07 mL) was added to the reaction mixture and the solvent was removed in vacuo. Purification by HPLC (Waters XBridge C8 19×150 mm 5 μm column, gradient 5-100% acetonitrile+0.2% ammonium hydroxide in water +0.2% ammonium hydroxide) provided the title compound. ¹H NMR (600 MHz, dimethylsulfoxide-d₆) δ ppm 12.87 (s, 1H), 8.87 (d, 1H), 8.70 (s, 1H), 7.55 (m, 2H), 7.46 (m, 1H), 7.21-7.10 (m, 6H), 7.05 (td, 1H), 6.94 (d, 1H), 6.87 (s, 1H), 6.75 (s, 1H), 6.11 (s, 1H), 5.71 (m, 1H), 5.26-5.10 (m, 2H), 4.65-4.45 (m, 2H), 4.34-4.22 (m, 1H), 3.86 (m, 1H), 3.77 (s, 3H), 3.54-3.37 (m, 11H), 3.23 (s, 3H), 2.83 (m, 2H), 2.66 (m, 1H), 2.53 (m, 1H), 2.21 (m, 3H), 2.03 (m, 1H), 1.86 (m, 1H), 1.60 (m, 1H), 1.52 (m, 1H), 1.40 (m, 2H), 1.28 (m, 1H), 1.16 (m, 1H), 1.07 (m, 1H). MS (APCI) m/z 1034.4 (M+H)⁺.

### Example 22

### (7R,16R)-19,23-dichloro-10-[(2-{4-[(2R)-2,3-dihydroxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 22A

### tert-butyl (7R,16R)-19,23-dichloro-10-{[2-(4-{[(4S)-2,2-dimethyl-1,3-dioxolan-4-yl]methoxy}phenyl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (100 mg), Example 18D (78 mg), triphenylphosphine (68.0 mg) and di-tert-butyl azodicarboxylate (56.9 mg). The vial was capped with a septum, evacuated and backfilled with nitrogen. Toluene (1.2 mL) was added. The vial was evacuated and backfilled with nitrogen again. The reaction mixture was stirred overnight. The mixture was concentrated and purified by silica gel flash chromatography on AnaLogix IntelliFlash²⁸⁰ system, eluting with 0-8% methanol in dichloromethane to afford the title compound. MS (ESI) m/z 1107.4 (M+H)⁺.

### Example 22B

### (7R,16R)-19,23-dichloro-10-[(2-{4-[(2R)-2,3-dihydroxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 22A for Example 18E in Example 18F. ¹H NMR (400 MHz, dimethylsulfoxide-d₆) δ ppm 8.83 (d, 1H), 8.76 (s, 1H), 8.45-8.27 (m, 2H), 7.43 (d, 1H), 7.25-7.11 (m, 4H), 7.10-7.03 (m, 2H), 6.91 (d, 1H), 6.82 (dd, 1H), 6.28 (dd, 1H), 5.79 (d, 1H), 5.21 (q, 2H), 5.01-4.85 (m, 1H), 4.54-4.37 (m, 2H), 4.09 (dd, 1H), 3.95 (dd, 1H), 3.89-2.82 (m, 17H), 2.80 (s, 3H), 1.99 (s, 3H), 1.96 (s, 3H). MS (ESI) m/z 1011.4 (M+H)⁺.

### Example 23

### (7R,16R)-19,23-dichloro-10-[(2-{2-[(2R)-2,3-dihydroxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 23A

### (S)-2-(2-((2,2-dimethyl-1,3-dioxolan-4-yl)methoxy)phenyl)-4,4,5,5-tetramethyl-1,3,2-dioxaborolane

To a solution of 2-(4,4,5,5-tetramethyl-1,3,2-dioxaborolan-2-yl)phenol (1.0 g) in N,N-dimethyl formamide (10 mL) was added (R)-(2,2-dimethyl-1,3-dioxolan-4-yl)methyl 4-methylbenzenesulfonate (1.43 g) and cesium carbonate (1.78 g). The mixture was stirred at 120° C. for 24 hours. The reaction was diluted with ethyl acetate and washed with water. The organic layer was dried over anhydrous sodium sulfate, filtered, and concentrated under reduced pressure. The residue was purified by silica gel chromatography (Biotage® Isolera, 25 g silica gel column), eluting with 0-80% ethyl acetate in heptane to give the title compound. MS (APCI) m/z 335.4 (M+H)⁺.

### Example 23B

### (S)-(2-(2-((2,2-dimethyl-1,3-dioxolan-4-yl)methoxy)phenyl)pyrimidin-4-yl)methanol

To a solution of (2-chloropyrimidin-4-yl)methanol (143 mg) and Example 23A (330 mg) in a solvent mixture of tetrahydrofuran (5.712 mL) and saturated aqueous sodium bicarbonate solution (3.26 mL) was added palladium (0) tetrakis(triphenylphosphine) (114 mg). The reaction was heated to 75° C. overnight. The reaction was cooled to room temperature and diluted with water (20 mL) and dichloromethane (20 mL). The layers were separated, and the aqueous layer was extracted with additional dichloromethane (2×25 mL). The combined organics were dried with anhydrous sodium sulfate, filtered and concentrated under reduced pressure. Toluene (3 mL) was added to the residue, and the toluene solution was purified by silica gel chromatography (Biotage® Isolera, 10 g silica gel column), eluting with a gradient of 0-50% ethyl acetate in heptane over 20 minutes, to give the title compound. MS (ESI) m/z 317.2 (M+H)⁺.

### Example 23C

### tert-butyl (7R,16R)-19,23-dichloro-10-{[2-(2-{[(4S)-2,2-dimethyl-1,3-dioxolan-4-yl]methoxy}phenyl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 23C was prepared according to the procedure for Example 17C, substituting Example 23B for Example 17B and substituting Example 16N for Example 12P. MS (ESI) m/z 1107.5 (M+H)⁺.

### Example 23D

### (7R,16R)-19,23-dichloro-10-[(2-{2-[(2R)-2,3-dihydroxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To an ambient solution of Example 23C (93 mg) in dichloromethane (0.5 mL) was added trifluoroacetic acid (0.5 mL), and the reaction was stirred for 6 hours. The reaction was concentrated under reduced pressure. The residue was dissolved in acetonitrile (3 mL) and water (0.5 mL) was added. To the mixture was added solid potassium carbonate in portions until the pH was basic (˜9). The mixture was treated with acetic acid (1.5 mL) and filtered through a 0.45 m syringe filter. The solution was purified by reverse phase HPLC (Phenomenex® Luna™ 25 0x50 mm column) eluting with a gradient of 20-75% acetonitrile in water containing 0.1% v/v trifluoroacetic acid over 45 minutes. The fractions containing product were lyophilized to give the title compound. ¹H NMR (500 MHz, dimethylsulfoxide-d₆) δ ppm 9.43 (s, 1H), 8.87 (d, 1H), 8.77 (s, 1H), 7.67 (dd, 1H), 7.54 (d, 1H), 7.51-7.45 (m, 1H), 7.24-7.13 (m, 6H), 7.09 (t, 1H), 6.88 (d, 1H), 6.84 (dd, 1H), 6.28 (dd, 1H), 5.79 (d, 1H), 5.23 (d, 1H), 5.17 (d, 1H), 4.98-4.85 (m, 1H), 4.55-4.39 (m, 2H), 4.12 (dd, 1H), 4.01 (dd, 1H), 3.77 (p, 1H), 3.67 (dd, 1H), 3.53-3.35 (m, 2H), 3.27-3.16 (m, 2H), 3.13-2.94 (m, 8H), 2.85 (qd, 2H), 2.80 (s, 3H), 2.01 (s, 3H), 1.95 (s, 3H). MS (ESI) m/z 1011.3 (M+H)⁺.

### Example 24

### (7R,16R,21S)-10-({2-[2-(carboxymethoxy)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 24A

### 2-(4-(dimethoxymethyl)pyrimidin-2-yl)phenol

2-Hydroxybenzimidamide hydrochloride (2.5 g) was dissolved in ethanol (60 mL). Sodium ethanolate (21% in ethanol, 10.81 mL, 9.39 g) was added, followed by (E)-4-(dimethylamino)-1,1-dimethoxybut-3-en-2-one (2.76 g). The reaction was stirred at 70° C. for 16 hours. The solvent was removed by rotary evaporation. The residue was taken up in 50% ethyl acetate in heptanes (100 mL). Saturated aqueous ammonium chloride (20 mL) was added and the layers were separated. The organic layer was washed with water (2×20 mL) and with brine (20 mL). The solution was dried on anhydrous sodium sulfate, and filtered. The solvent was removed under vacuum to yield the title compound. ¹H NMR (400 MHz, dimethylsulfoxide-d₆) δ ppm 13.15 (s, 1H), 9.03 (d, 1H), 8.41 (dd, 1H), 7.55 (d, 1H), 7.44 (td, 1H), 7.01 (dd, 1H), 6.99 (d, 1H), 5.49 (s, 1H), 3.40 (s, 6H). MS (ESI) m=245 (M−H)⁻.

### Example 24B

### 2-(4-(hydroxymethyl)pyrimidin-2-yl)phenol

Example 24A (1.5 g) was dissolved in 1,4-dioxane (25 mL). Aqueous hydrogen chloride (2 M, 25 mL) was added and the solution was heated to 50° C. for 16 hours. The solution was cooled to room temperature and further cooled to 0° C. using an ice bath. The pH of the solution was adjusted to eight using concentrated aqueous sodium hydroxide. To the solution was added sodium tetrahydroborate (0.461 g) in three portions five minutes apart. The solution was mixed at 0° C. for two hours. While keeping the reaction at 0° C. 10 mL of ethyl acetate was added, and the solution was stirred for 10 minutes. The solution was then diluted further with ethyl acetate (20 mL), keeping the reaction at 0° C. Saturated aqueous ammonium chloride (5 mL) was added, and the solution was stirred for 10 minutes.

The phases were separated. The aqueous layer was adjusted to pH 5 using 2 M aqueous HCl. The aqueous layer was extracted once with ethyl acetate (20 mL). The organic portions were combined and dried over anhydrous sodium sulfate. After filtration, the solution was concentrated under vacuum and purified by flash column chromatography on silica gel using a gradient of 60-80% ethyl acetate in heptanes. The solvent was removed by rotary evaporation to yield the title compound. ¹H NMR (400 MHz, dimethylsulfoxide-d₆) δ ppm 13.29 (s, 1H), 8.93 (d, 1H), 8.40 (dd, 1H), 7.54 (d, 1H), 7.41 (td, 1H), 6.98-6.94 (m, 2H), 5.78 (t, 1H), 4.69 (d, 2H). MS (ESI) m=203 (M+H)⁺.

### Example 24C

### 2-(4-(((tert-butyldimethylsilyl)oxy)methyl)pyrimidin-2-yl)phenol

Example 24B (1000 mg) was dissolved in tetrahydrofuran (12 mL). 1H-Imidazole (741 mg) was added and the solution was cooled to 0° C. tert-Butylchlorodimethylsilane (820 mg) dissolved in tetrahydrofuran (6 mL) was added. The solution was stirred at 0° C. for five minutes, and allowed to warm to room temperature. Additional tetrahydrofuran (10 mL) was added, and the solution was stirred at room temperature for 16 hours. Saturated aqueous ammonium chloride (5 mL) was added. The solution was extracted with ethyl acetate (2×20 mL). The organic extracts were combined and were washed with water (10 mL) and brine (10 mL). The solution was dried over anhydrous sodium sulfate, and filtered. The solution was concentrated under vacuum and purified by flash column chromatography on silica gel using a gradient of 20-100% ethyl acetate in heptanes. The solvent was removed by rotary evaporation to yield the title compound. ¹H NMR (400 MHz, dimethylsulfoxide-d₆) δ ppm 13.21 (s, 1H), 8.95 (d, 1H), 8.38 (dd, 1H), 7.48 (d, 1H), 7.41 (td, 1H), 6.96 (d, 1H), 6.95 (dd, 1H), 4.88 (s, 2H), 0.94 (s, 9H), 0.14 (s, 6H). MS (APCI) m/z 317 (M+H)⁺.

### Example 24D

### tert-butyl 2-(2-(4-(((tert-butyldimethylsilyl)oxy)methyl)pyrimidin-2-yl)phenoxy)acetate

Example 24C (300 mg) was dissolved in tetrahydrofuran (6.5 mL). Sodium hydride (60%, 41.7 mg) was added, and the solution was mixed at room temperature for five minutes, tert-Butyl 2-bromoacetate (203 mg) was added, and the solution was mixed at room temperature overnight. The solution was diluted with ethyl acetate (15 mL), saturated aqueous ammonium chloride (2 mL), and water (0.5 mL). The layers were separated and the organic layer was washed with brine. The solution was dried over anhydrous sodium sulfate, filtered, and concentrated under vacuum. The crude material was carried on in the next step without further purification. MS (ESI) m/z 431.2 (M+H)⁺.

### Example 24E

### tert-butyl 2-(2-(4-(hydroxymethyl)pyrimidin-2-yl)phenoxy)acetate

Example 24D (408 mg) was dissolved in tetrahydrofuran (4 mL). Acetic acid (171 mg) was added, followed by 1 M tetrabutylammonium fluoride in tetrahydrofuran (495 mg). The solution was stirred for one hour at room temperature and concentrated under vacuum. The material was purified by flash column chromatography on silica gel using a gradient of 50-70% ethyl acetate in heptanes. The solvent was removed by rotary evaporation to yield the title compound. ¹H NMR (500 MHz, dimethylsulfoxide-d₆) δ ppm 8.85 (d, 1H), 7.55 (dd, 1H), 7.50 (d, 1H), 7.42 (td, 1H), 7.08 (td, 1H), 7.00 (d, 1H), 5.66 (m, 1H), 4.63 (s, 2H), 4.59 (s, 2H), 1.40 (s, 9H). MS (ESI) m/z 314.9 (M−H)⁻.

### Example 24F

### (7R,16R,21S)-10-({2-[2-(carboxymethoxy)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 24E for Example 19A in Example 19B. ¹H NMR (500 MHz, dimethylsulfoxide-d₆) δ ppm 9.43 (s, 1H), 8.92 (d, 1H), 8.75 (s, 1H), 7.67 (dd, 1H), 7.56 (d, 1H), 7.47 (td, 1H), 7.23-7.11 (m, 7H), 6.97 (d, 1H), 6.90 (d, 1H), 6.84 (dd, 1H), 6.16 (m, 1H), 5.67 (d, 1H), 5.20 (q, 2H), 4.75 (s, 2H), 4.58 (m, 1H), 4.46 (d, 1H), 4.36 (dd, 1H), 3.88 (dd, 1H), 3.08 (m, 4H), 2.93-2.84 (m, 3H), 2.78 (s, 3H), 2.73 (m, 2H), 2.45-2.37 (m, 2H), 2.23 (s, 3H). MS (ESI) m/z 947.1 (M+H)⁺.

### Example 25

### (7R,16R)-19,23-dichloro-1-(4-fluorophenyl)-20,22-dimethyl-10-{[2-(4-methyl-4-oxo-1,4λ⁵-azaphosphinan-1-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 25A

### 1-(4-(hydroxymethyl)pyrimidin-2-yl)-4-methyl-1,4-azaphosphinane 4-oxide

A mixture of (2-chloropyrimidin-4-yl)methanol (200 mg), 4-methyl-1,4-azaphosphinane 4-oxide hydrochloride (258 mg) and triethylamine (579 μL) in 4.6 mL of acetonitrile was heated to 100° C. in a sealed tube for 16 hours. The mixture was concentrated under vacuum and the residue was purified by silica gel chromatography on a CombiFlash® Teledyne Isco system eluting with 0-20% methanol containing 7N ammonia in dichloromethane to provide the title compound. ¹H NMR (501 MHz, CDCl₃) δ ppm 8.29 (d, 1H), 6.54 (d, 1H), 4.60 (d, 2H), 4.30 (dddd, 2H), 4.13 (dddd, 2H), 3.45 (t, 1H), 2.12-1.95 (m, 2H), 1.87 (dtd, 2H), 1.58 (d, 3H). MS (ESI) m/z 242.3 (M+H)⁺.

### Example 25B

### (2-(4-methyl-4-oxido-1,4-azaphosphinan-1-yl)pyrimidin-4-yl)methyl methanesulfonate

To a solution of Example 25A (29 mg) and triethylamine (0.050 mL) in dichloromethane (1.2 mL) cooled to 0° C. was added methanesulfonyl chloride (0.012 mL), and the mixture was stirred at 0° C. for 30 minutes. The reaction mixture was diluted with dichloromethane (10 mL) and washed with brine (10 mL). The organic layer was dried over anhydrous sodium sulfate, filtered and concentrated under vacuum to give the title compound which was used in the next step without further purification. MS (ESI) m/z 320.1 (M+H)⁺.

### Example 25C

### tert-butyl (7R,16R)-19,23-dichloro-1-(4-fluorophenyl)-20,22-dimethyl-10-{[2-(4-methyl-4-oxo-1,4λ⁵-azaphosphinan-1-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 16N (27 mg) and Example 25B (19.2 mg) in N,N-dimethylformamide (0.15 mL) was added cesium carbonate (66.9 mg). The mixture was stirred at room temperature overnight. The mixture was diluted with saturated aqueous sodium bicarbonate solution (5 mL) and extracted with dichloromethane (3×10 mL). The combined organic layers were washed with brine, dried over anhydrous sodium sulfate, filtered and concentrated. The residue was purified by silica gel chromatography on a CombiFlash® Teledyne Isco system eluting with 0-15% methanol containing 7N ammonia in dichloromethane to provide the title compound. ¹H NMR (400 MHz, CDCl₃) δ ppm 8.61 (s, 1H), 8.36 (d, 1H), 7.07-7.01 (m, 2H), 6.97-6.88 (m, 3H), 6.75-6.64 (m, 2H), 5.94 (dd, 1H), 5.89 (d, 1H), 5.02 (q, 1H), 4.95-4.82 (m, 2H), 4.51 (dd, 1H), 4.43-4.26 (m, 3H), 4.10-3.96 (m, 2H), 3.49 (dd, 1H), 3.05 (d, 1H), 2.89 (dd, 1H), 2.73-2.55 (m, 5H), 2.48 (s, 4H), 2.31 (s, 3H), 2.15 (s, 3H), 2.10-1.97 (m, 2H), 1.95 (s, 3H), 1.93-1.79 (m, 2H), 1.59 (d, 3H), 1.21 (s, 9H). MS (ESI) m/z 1032.4 (M+H)⁺.

### Example 25D

### (7R,16R)-19,23-dichloro-1-(4-fluorophenyl)-20,22-dimethyl-10-{[2-(4-methyl-4-oxo-1,4λ⁵-azaphosphinan-1-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 25C (17 mg) in dichloromethane (1 mL) was added trifluoroacetic acid (1 mL) and the reaction was stirred at room temperature for 5 hours. The reaction mixture was concentrated and the residue was purified by reversed-phase HPLC on a Gilson PLC 2020 using a Luna™ column (250×50 mm, 10 mm, 10-80% over 30 minutes with acetonitrile in water containing 0.1% trifluoroacetic acid) to provide the title compound after lyophilization. ¹H NMR (400 MHz, dimethyl sulfoxide-d₆) δ ppm 8.76 (s, 1H), 8.39 (d, 1H), 7.27-7.08 (m, 4H), 6.94-6.74 (m, 3H), 6.24 (dd, 1H), 5.78 (d, 1H), 5.06-4.88 (m, 3H), 4.53-4.39 (m, 2H), 4.26 (ddt, 2H), 3.76 (tdd, 2H), 3.02-2.92 (m, 1H), 2.88 (q, 2H), 2.80 (s, 3H), 1.98 (s, 3H), 1.96 (s, 3H), 1.94-1.83 (m, 2H), 1.77-1.63 (m, 2H), 1.54 (d, 3H). MS (ESI) m/z 973.9 (M−H)⁻.

### Example 26

### (7R,16R)-19,23-dichloro-1-(4-fluorophenyl)-10-({2-[2-(S-methanesulfonimidoyl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 26A

### (2-(2-(methylsulfinyl)phenyl)pyrimidin-4-yl)methanol

A 100 mL round bottom flask, equipped with stir bar, was charged with (2-chloropyrimidin-4-yl)methanol (1.571 g), 2-(methylsulfinyl)phenylboronic acid (2.00 g), tris(dibenzylideneacetone) dipalladium (0) (0.100 g), 1,3,5,7-tetramethyl-6-phenyl-2,4,8-trioxa-6-phosphaadamantane (0.064 g) and potassium phosphate (11.530 g). The flask was capped with a septum then evacuated and backfilled with nitrogen twice. Tetrahydrofuran (40 mL) and water (10 mL) was added and evacuated and backfilled with nitrogen twice. The mixture was stirred at 60° C. for 1 day. Water was added and the mixture was extracted with twice ethyl acetate. The organics were dried over magnesium sulfate, filtered, and concentrated. The residue was purified by silica gel flash chromatography on AnaLogix IntelliFlash²⁸⁰ system eluting with 1-10% methanol in dichloromethane to give the title compound. LC/MS (APCI) m/z 249.32 (M+H)⁺.

### Example 26B

### 4-(((tert-butyldimethylsilyl)oxy)methyl)-2-(2-(methylsulfinyl)phenyl)pyrimidine

To a flask containing Example 26A (1.92 g) in dichloromethane (75 mL) was added tert-butyldimethylsilyl chloride (1.282 g) followed by imidazole (0.579 g). The resulting mixture was stirred for 2 hours. The mixture was purified by silica gel flash chromatography on AnaLogix IntelliFlash²⁸⁰ system eluting with 15-80% ethyl acetate in hexanes to give the title compound. LC/MS (APCI) m/z 363.31 (M+H)⁺.

### Example 26C

### tert-butyl [{2-[4-({[tert-butyl(dimethyl) silyl]oxy}methyl)pyrimidin-2-yl]phenyl}(methyl)oxo-2λ⁶-sulfanylidene]carbamate

To a suspension of Example 26B (1500 mg), tert-butyl carbamate (969 mg), magnesium oxide (1026 mg) and rhodium (ii)acetate dimer (183 mg) in dichloromethane (50 mL) was added (diacetoxyiodo)benzene(2665 mg). The mixture was stirred at 40° C. for 3 days. The reaction mixture was filtered to remove material and the filtrate was concentrated in vacuo. The residue was purified by silica gel flash chromatography on AnaLogix IntelliFlash²⁸⁰ system eluting with 10-75% ethyl acetate in hexanes to give the title compound. LC/MS (APCI) m/z 478.3 (M+H)⁺.

### Example 26D

### tert-butyl [{2-[4-(hydroxymethyl)pyrimidin-2-yl]phenyl}(methyl)oxo-2λ6-sulfanylidene]carbamate

To a solution of Example 26C (102 mg) in methanol (1.6 mL) was added cesium fluoride (97 mg). The mixture was stirred for 1 day. The reaction mixture was concentrated and purified by silica gel flash chromatography on AnaLogix IntelliFlash²⁸⁰ system eluting with 10-90% ethyl acetate in hexanes to give the title compound. LC/MS (APCI) m/z 364.2 (M+H)⁺.

### Example 26E

### tert-butyl (7R,16R)-10-[(2-{2-[N-tert-butoxy (oxo) methane-S-methanesulfonimidoyl]phenyl}pyrimidin-4-yl)methoxy]-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (56 mg), Example 26D (50.3 mg), triphenylphosphine (36.3 mg) and di-tert-butyl azodicarboxylate (31.8 mg). The vial was capped with a septum and evacuated and backfilled with nitrogen. Toluene (1 mL) was added. The vial was evacuated and backfilled with nitrogen again. The reaction mixture was stirred for 1 day. The reaction mixture was concentrated and purified by silica gel flash chromatography on AnaLogix IntelliFlash²⁸⁰ system eluting with 0-8% methanol in dichloromethane to give the title compound. MS (ESI) m/z 1054.3 (M+H)⁺.

### Example 26F

### (7R,16R)-19,23-dichloro-1-(4-fluorophenyl)-10-({2-[2-(S-methanesulfonimidoyl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 26E (60 mg) in dichloromethane (0.3 mL) was added trifluoroacetic acid (0.30 mL). The mixture was stirred at ambient temperature for 1 day and concentrated in vacuo. The residue was dissolved in acetonitrile (1.5 mL) and dimethylformamide (0.5 mL) and purified by reverse phase prep LC using Gilson 2020 system (Luna™, C-18, 250×50 mm column, Mobile phase A: 0.1% trifluoroacetic acid in water: B: acetonitrile: 20-75% B to A gradient at 70 mL/minute) to afford the title compound after lyophilization. ¹H NMR (400 MHz, dimethylsulfoxide-d₆) δ ppm 8.93 (d, 1H), 8.77 (s, 1H), 8.19 (d, 1H), 7.93-7.70 (m, 3H), 7.63 (d, 1H), 7.27-7.09 (m, 4H), 6.90 (d, 1H), 6.83 (dd, 1H), 6.28 (dd, 1H), 5.79 (d, 1H), 5.30-5.11 (m, 2H), 4.96 (tt, 1H), 4.47 (td, 2H), 3.97-2.82 (m, 16H), 2.80 (s, 3H), 2.00 (s, 3H), 1.96 (s, 3H). MS (ESI) m/z 998.5 (M+H)⁺.

### Example 27

### (7R,16R,21R)-19-chloro-1-(4-fluorophenyl)-16-[(4-{2-[2-(2-methoxyethoxy)ethoxy]ethyl}piperidin-1-yl)methyl]-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was isolated as a minor product during the synthesis of Example 21E. ¹H NMR (600 MHz, dimethylsufoxide-d₆) δ ppm 13.36 (s, 1H), 9.30 (s, 1H), 8.87 (d, 1H), 8.62 (s, 1H), 7.68 (d, 1H), 7.50 (dd, 1H), 7.44 (m, 1H), 7.31 (m, 2H), 7.22 (m, 2H), 7.13 (m, 1H), 7.02 (m, 2H), 6.88 (m, 1H), 6.82 (m, 1H), 6.69 (m, 1H), 6.12 (d, 1H), 5.78 (m, 1H), 5.35-5.30 (m, 1H), 5.24-5.15 (m, 2H), 4.27-4.24 (m, 1H), 4.15-4.13 (m, 1H), 3.86-3.78 (m, 1H), 3.73 (s, 3H), 3.71-3.56 (m, 2H), 3.53-3.42 (m, 11H), 3.24 (s, 3H), 3.19-3.09 (m, 1H), 2.57-2.53 (m, 1H), 2.50 (m, 5H), 1.94-1.89 (m, 2H), 1.70-1.62 (m, 1H), 1.50-1.40 (m, 4H). MS (APCI) m/z 1034.4 (M+H)⁺.

### Example 28

### (7R,16R)-19-chloro-1-(4-fluorophenyl)-20-methyl-10-{[2-(1-methyl-6-oxo-1,6-dihydropyridin-2-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 28A

### 6-bromo-1-methylpyridin-2(1H)-one

To a solution of 6-bromopyridin-2(1H)-one (9 g) in acetonitrile (500 mL) was added K₂CO₃ (15.7 g) and iodomethane (15.4 g) at 25° C. The reaction mixture was stirred at 25° C. for 10 hours and filtered. The filtrate was concentrated and the residue was purified by column chromatography on silica gel (eluting with petroleum ether:ethyl acetate=5:1) to give the title compound. ¹H NMR (400 MHz, CDCl₃) δ ppm 7.12 (dd, 1H), 6.54-6.43 (m, 2H), 3.72 (s, 3H).

### Example 28B

### 1-methyl-6-(4,4,5,5-tetramethyl-1,3,2-dioxaborolan-2-yl) pyridin-2(1H)-one

To a solution of Example 28A (11 g) in 1,4-dioxane (180 mL) were added 4,4,4′,4′,5,5,5′,5′-octamethyl-2,2′-bi(1,3,2-dioxaborolane) (17.8 g), potassium acetate (17.23 g) and (1,1′-bis(diphenylphosphino)ferrocene dichloro palladium (II) dichloromethane complex (5.14 g) at room temperature under nitrogen flow. The reaction mixture was stirred at 110° C. for 3 hours under nitrogen atmosphere, cooled to 25° C. and filtered. The filter cake was washed with warm toluene (40° C., 2×100 mL). The combined organic phases were concentrated under reduced pressure to provide the title compound.

### Example 28C

### methyl 2-(1-methyl-6-oxo-1,6-dihydropyridin-2-yl)pyrimidine-4-carboxylate

To a solution of Example 28B (18 g) in 1,4-dioxane (150 mL) were added methyl 2-chloropyrimidine-4-carboxylate, potassium phosphate (21.5 g) and (1, l′-bis(diphenylphosphino) ferrocene dichloro palladium (II) dichloromethane complex (2.54 g) at room temperature under nitrogen flow. The reaction mixture was stirred at 110° C. for 3 hours under nitrogen atmosphere, cooled and filtered. The filtrate was concentrated and the residue was purified by flash column chromatography on silica gel (eluting with ethyl acetate:methanol=10:1) to provide the title compound. ¹H NMR (400 MHz, dimethylsulfoxide-d₆) δ ppm 9.25 (dd, 1H), 8.10 (dd, 1H), 7.62-7.41 (m, 1H), 6.67-6.52 (m, 2H), 3.95 (d, 3H), 3.42 (d, J=1.1 Hz, 3H).

### Example 28D

### 6-(4-(hydroxymethyl)pyrimidin-2-yl)-1-methylpyridin-2(1H)-one

To a solution of Example 28C (1.5 g) in a mixture of methanol (10 mL), N,N-dimethylformamide (10 mL) and water (1 mL) was added NaBH₄ (0.347 g) at room temperature. The reaction was stirred at 0° C. for 2 hours under nitrogen atmosphere, quenched by addition of 2 mL of acetic acid and concentrated. The residue was purified by column chromatography (eluting with chloroform:methanol=10:1) to give the title compound. ¹H NMR (400 MHz, methanol-d₄) δ ppm 8.91 (d, 1H), 7.69 (d, 1H), 7.61 (dd, 1H), 6.74-6.64 (m, 2H), 4.75 (s, 2H), 3.55 (s, 3H).

### Example 28E

### tert-butyl (7R,16R)-19-chloro-1-(4-fluorophenyl)-20-methyl-10-{[2-(1-methyl-6-oxo-1,6-dihydropyridin-2-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 12P (25 mg), Ph₃P (34.5 mg) and Example 28D (24.97 mg) was added toluene (0.6 mL) and tetrahydrofuran (0.6 mL). The mixture was stirred for 1 minute and (E)-di-tert-butyl diazene-1,2-dicarboxylate (30.2 mg) was added. The mixture was stirred at ambient temperature overnight and concentrated. The residue was purified by flash chromatography on a Teledyne Isco CombiFlash® system, eluting with 0-10% methanol in dichloromethane to provide the title compound. MS (APCI) m/z 960.3 (M+H)⁺.

### Example 28F

### (7R,16R)-19-chloro-1-(4-fluorophenyl)-20-methyl-10-{[2-(1-methyl-6-oxo-1,6-dihydropyridin-2-yl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 28E (20 mg) in dichloromethane (2 mL) was treated with TFA (1 mL) overnight and concentrated. The residue was purified by reverse phase HPLC on an ACCQPrep HP125 system, eluting with 35-60% acetonitrile in 0.1 TFA water solution to provide the title compound as a TFA salt. ¹H NMR (400 MHz, dimethylsulfoxide-d₆) δ ppm 9.46 (s, br, 1H), 9.01 (d, 1H), 8.75 (s, 1H), 7.70 (d, 1H), 7.54 (dd, 1H), 7.26-7.09 (m, 5H), 6.94 (dd, 2H), 6.84 (dd, 1H), 6.67-6.52 (m, 2H), 6.15 (dd, 1H), 5.67 (d, 1H), 5.23 (q, 2H), 4.61 (q, 1H), 4.52-4.30 (m, 2H), 3.86 (dd, 1H), 3.16-2.84 (m, 6H), 2.78 (d, 6H), 2.38 (s, 1H), 2.22 (s, 3H).

### Example 29

### (7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclopropyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 29A

### 4-(dimethoxymethyl)-2-(methylthio)pyrimidine

An oven-dried three-neck round bottom flask was charged with solid sodium methoxide (8.73 g) and cooled into an ice brine water bath. Methanol (128 mL) was added and thiourea (13.18 g) was added portionwise over the course of 15 minutes. The mixture was stirred at 2° C. for 60 minutes then 4-(dimethylamino)-1,1-dimethoxybut-3-en-2-one (20 g) was added dropwise via syringe over 5 minutes. The cooling bath was removed and the reaction was heated to reflux for 4 hours (internal temperature at 65° C.). The reaction was cooled to 9° C. with an ice bath and methyl iodide (9.75 mL) was slowly added. The cooling bath was removed and the mixture allowed to stir at room temperature overnight. The reaction mixture was filtered through a disposable plastic funnel and rinsed with methanol. The filtrate was concentrated and added to ˜800 mL of ethyl acetate and poured into a separatory funnel. The mixture was washed with water and brine. The organic layer was dried over anhydrous magnesium sulfate, filtered and concentrated onto silica gel. Purification by flash chromatography on a CombiFlash® Teledyne Isco system using a Teledyne Isco RediSep® Rf gold 330 g silica gel column (eluting 15-50% ethyl acetate/heptane) afforded the title compound. ¹H NMR (400 MHz, dimethylsulfoxide-d₆) δ ppm 8.68 (d, 1H), 7.22 (d, 1H), 5.21 (s, 1H), 3.35 (s, 6H), 2.51 (s, 3H).

### Example 29B

### 4-(dimethoxymethyl)-2-(methylsulfonyl)pyrimidine

Example 29A (14.6 g) was dissolved in methanol (122 mL) and water (122 mL) and the stirring mixture was cooled with an ice bath then oxone (potassium peroxomonosulfate) (67.2 g) was added portionwise over 15 minutes. The resulting mixture was stirred at 0° C. for 3 hours, and the cooling bath was removed to allow for the reaction to stir at room temperature for an additional 2 hours. The mixture was concentrated to remove most of the methanol and the remaining aqueous mixture was filtered and washed with ˜200 mL of methylene chloride. The biphasic mixture was poured into a separatory funnel, the organic layer was removed and the aqueous layer was washed with one portion of methylene chloride. The organic layers were combined, dried over anhydrous magnesium sulfate, filtered and concentrated onto silica gel. Purification by flash chromatography on a CombiFlash® Teledyne Isco system using a Teledyne Isco RediSep® Rf gold 330 g silica gel column (eluting 50-100% ethyl acetate/heptane) afforded the title compound. MS (APCI) m=233.3 (M+H)⁺.

### Example 29C

### methyl 2-(4-(dimethoxymethyl)pyrimidin-2-yl)acetate

Sodium hydride (2.262 g, 60% in mineral oil) was added in small portions to an ice bath cooled stirring solution of tert-butyl methyl malonate (15.15 mL) in N,N-dimethyl formamide (65.3 mL). The cooling bath was removed and the mixture was stirred at room temperature under nitrogen for 20 minutes after which Example 29B (10.4 g) was added as a dimethylformamide (9.33 mL) solution. The resulting mixture was then stirred at 80° C. for 45 minutes. After cooling to ambient temperature, the mixture was poured into 300 mL of saturated aqueous ammonium chloride, transferred into a separatory funnel and extracted with two portions of diethyl ether. The combined organic layers were washed with water and brine, dried over anhydrous magnesium sulfate, filtered and concentrated. The residue was dissolved in 50 mL of dichloromethane and stirred at 0° C. Trifluoroacetic acid (35 mL, 454 mmol) was added dropwise with an addition funnel and stirring was continued at 0° C. for 10 minutes. The cooling bath was removed and the mixture was allowed to stir at room temperature for 1 hour before being concentrated and transferred into a separatory funnel containing 300 mL of saturated aqueous sodium bicarbonate. The mixture was extracted with four portions of ethyl acetate, and the combined layers was dried over anhydrous magnesium sulfate, filtered and concentrated onto silica gel. Purification by flash chromatography on a CombiFlash® Teledyne Isco system using a Teledyne Isco RediSep® Rf gold 220 g silica gel column (eluting 20-100% ethyl acetate/heptane) afforded the title compound. MS (APCI) m/z 227.4 (M+H)⁺.

### Example 29D

### methyl 1-(4-(dimethoxymethyl)pyrimidin-2-yl) cyclopropanecarboxylate

To a stirring solution of Example 29C (6 g) and 1,2-dibromoethane (7.47 g) in dry N,N-dimethylformamide (332 mL) at 0° C. was added cesium carbonate (34.6 g) in one portion. The mixture was stirred at 0° C. for 2 hours and the cooling bath was removed to allow the mixture to stir at room temperature overnight. The stirring mixture was put under high vacuum for 24 hours to remove most of the N,N-dimethylformamide. The crude residue obtained was dissolved into a mixture of water and ethyl acetate (200 mL each) and the mixture was poured into a 500 mL separatory funnel. The mixture was partitioned between the two phases then the organic layer was removed and the aqueous phase was extracted with two portions of ethyl acetate. The organic layers were combined then washed with water, dried over anhydrous magnesium sulfate, filtered and concentrated onto silica gel. Purification by flash chromatography on a CombiFlash® Teledyne Isco system using a Teledyne Isco RediSep® Rf gold 220 g silica gel column (eluting 10-100% ethyl acetate/heptane) afforded the title compound. MS (APCI) m/z 253.4 (M+H)⁺.

### Example 29E

### (1-(4-(dimethoxymethyl)pyrimidin-2-yl)cyclopropyl)methanol

To a stirring solution of Example 29D (3 g), at 0° C., was slowly added a 1 molar solution (in toluene) of diisobutylaluminum hydride (65.4 mL) and the mixture was stirred at 0° C. After 30 minutes the reaction was quenched with water (50 mL) followed by saturated aqueous Rochelle's salt (50 mL) and the resulting mixture was stirred vigorously for 30 minutes before it was transferred into a separatory funnel and diluted with ethyl acetate. The organic layer was removed and the aqueous layer was extracted with three portions of ethyl acetate. The combined organic layers was dried over anhydrous magnesium sulfate, filtered and concentrated onto silica gel. Purification by flash chromatography on a CombiFlash® Teledyne Isco system using a Teledyne Isco RediSep® Rf gold 80 g silica gel column (eluting 30-100% ethyl acetate/heptane) afforded the title compound. MS (APCI) m/z 225.4 (M+H)⁺.

### Example 29F

### 2-(1-(2,5,8,11-tetraoxadodecyl)cyclopropyl)-4-(dimethoxymethyl)pyrimidine

To a stirring solution of Example 29E (70 mg) and 2-(2-(2-methoxyethoxy)ethoxy)ethyl 4-methylbenzenesulfonate (199 mg) in acetonitrile (3.1 mL) was slowly added sodium hydride (14.98 mg) and the mixture was stirred at 45° C. overnight. After cooling to ambient temperature, the reaction was quenched with 4 drops of saturated aqueous ammonium chloride and the mixture was concentrated onto silica gel. Purification by flash chromatography on a CombiFlash® Teledyne Isco system using a Teledyne Isco RediSep® Rf gold 24 g silica gel column (solvent A=2:1 ethyl acetate:ethanol; solvent B=heptane, eluting 0-50% A to B) afforded the title compound. MS (APCI) m/z 371.4 (M+H)⁺.

### Example 29G

### 2-(1-(2,5,8,11-tetraoxadodecyl)cyclopropyl)pyrimidine-4-carbaldehyde

To a stirring mixture of Example 29F (80 mg) in tetrahydrofuran (1.35 mL) was added aqueous HCl (1.3 mL) and the mixture was stirred at 55° C. for 5 hours. After cooling to ambient temperature, the mixture was poured into a separatory funnel containing saturated aqueous sodium bicarbonate. The mixture was extracted with 5 portions of dichloromethane The organic layers were combined and dried over anhydrous magnesium sulfate, filtered and concentrated to obtain the crude title compound which was carried through the next step without further purification. MS (APCI) m/z 325.2 (M+H)⁺.

### Example 29H

### (2-(1-(2,5,8,11-tetraoxadodecyl)cyclopropyl)pyrimidin-4-yl)methanol

To a stirring solution of Example 29G (70.1 mg) in tetrahydrofuran (1.5 mL) was added 17 mg of sodium borohydride in one portion followed by 0.5 mL of methanol. The mixture was stirred at room temperature for 30 minutes and quenched by careful addition of 10 drops of saturated aqueous ammonium chloride solution. The resulting mixture was concentrated onto silica gel. Purification by flash chromatography on a CombiFlash® Teledyne Isco system using a Teledyne Isco RediSep® Rf gold 12 g silica gel column (eluting with solvent A=2:1 ethyl acetate:ethanol; solvent B=heptane, eluting 20-75% A to B) afforded the title compound. MS (APCI) m/z 327.2 (M+H)⁺.

### Example 291

### tert-butyl (7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclopropyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

A reaction vessel equipped with stir bar was charged with Example 16N (73 mg), Example 29H (59 mg) and triphenylphosphine (50 mg). The vial was capped with a septa and evacuated and backfilled with nitrogen twice. Toluene (0.9 mL) was added and the mixture was cooled with an ice bath. To the stirring mixture. (E)-di-tert-butyl diazene-1,2-dicarboxylate (42 mg) was added in one portion and the vial was capped with a septa. The stirring mixture evacuated and backfilled with nitrogen twice. The stirring continued at 0° C. for 10 minutes, the cooling bath was removed and the mixture allowed to stir at room temperature overnight. The mixture was concentrated onto silica gel and purification by flash chromatography on a CombiFlash® Teledyne Isco system using a Teledyne Isco RediSep® Rf gold 12 g silica gel column (eluting with 0-20% methanol/dichloromethane) afforded the title compound. MS (APCI) m/z 1117.4 (M+H)⁺.

### Example 29J

### (7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclopropyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 291 (80 mg) in dichloromethane (0.75 mL) was added trifluoroacetic acid (0.75 mL) and the reaction mixture was stirred at room temperature for 5 hours and concentrated. The crude residue was redissolved into 2 mL of acetonitrile and purified directly by reverse phase prep LC using a Gilson 2020 system (Luna™, C-18, 250×50 mm column, Mobile phase A: 0.1% trifluoroacetic acid in H₂O; B: acetonitrile; 5-75% B to A gradient at 75 mL/minute, 30 minute gradient) to afford the title compound as a trifluoroacetic acid salt. ¹H NMR (400 MHz, dimethylsulfoxide-d₆) δ ppm 8.75 (s, 1H), 8.66 (d, 1H), 7.33 (d, 1H), 7.24-7.10 (m, 4H), 6.90-6.77 (m, 2H), 6.24 (dd, 1H), 5.77 (d, 1H), 5.17-4.98 (m, 2H), 4.98-4.84 (m, 1H), 4.55-4.35 (m, 2H), 3.94-3.84 (m, 2H), 3.66-3.30 (m, 14H), 3.21 (s, 3H), 3.19-2.91 (m, 6H), 2.90-2.82 (m, 2H), 2.80 (s, 3H), 2.49-2.38 (m, 2H), 1.98 (s, 3H), 1.95 (s, 3H), 1.22 (q, 2H), 1.06 (q, 2H). MS (APCI) m/z 1061.3 (M+H)⁺.

### Example 30

### (7R,16R)-19,23-dichloro-1-(4-fluorophenyl)-10-({2-[4-(S-methanesulfonimidoyl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 30A

### (2-(4-(methylsulfinyl)phenyl)pyrimidin-4-yl)methanol

A 100 mL round bottom flask, equipped with stir bar, was charged with (2-chloropyrimidin-4-yl)methanol (1.571 g), 4-(methanesulfinyl)benzeneboronic acid (2.000 g), tris(dibenzylideneacetone) dipalladium (0) (0.100 g), 1,3,5,7-tetramethyl-6-phenyl-2,4,8-trioxa-6-phosphaadamantane (0.064 g) and potassium phosphate (11.53 g). The flask was capped with a septum and evacuated and backfilled with nitrogen twice. Tetrahydrofuran (40 mL) and water (10 mL) were added, and the flask was evacuated and backfilled with nitrogen twice again. The mixture was stirred at 60° C. for 1 day. Water was added and the mixture was extracted twice with ethyl acetate. The organics were dried over magnesium sulfate, filtered, and concentrated. The residue was purified by silica gel flash chromatography on AnaLogix IntelliFlash²⁸⁰ system eluting with 1-10% methanol in dichloromethane to give the title compound. MS (ESI) m/z 249.3 (M+H)⁺.

### Example 30B

### tert-butyl [{4-[4-(hydroxymethyl)pyrimidin-2-yl]phenyl}(methyl)oxo-λ⁶-sulfanylidene]carbamate

To a suspension of Example 30A (300 mg), tert-butyl carbamate (212 mg), magnesium oxide (200 mg) and rhodium (ii)acetate dimer (21.36 mg) in dichloromethane (10 mL) was added (diacetoxyiodo)benzene (584 mg). The mixture was stirred at 45° C. for 1 day. The reaction mixture was filtered to remove the material and the filtrate was concentrated in vacuo. The residue was purified by silica gel flash chromatography on AnaLogix IntelliFlash²⁸⁰ system eluting with 4-100% ethyl acetate in hexanes to give the title compound. MS (ESI) m/z 363.8 (M+H)⁺.

### Example 30C

### tert-butyl (7R,16R)-10-[(2-{4-[N-tert-butoxy (oxo) methane-S-methanesulfonimidoyl]phenyl}pyrimidin-4-yl)methoxy]-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 30B for Example 26D in Example 26E. MS (ESI) m/z 1154.4 (M+H)⁺.

### Example 30D

### (7R,16R)-19,23-dichloro-1-(4-fluorophenyl)-10-({2-[4-(S-methanesulfonimidoyl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 30C for Example 26E in Example 26F. ¹H NMR (400 MHz, dimethylsulfoxide-d₆) δ ppm 8.98 (d, 1H), 8.75 (s, 1H), 8.61 (d, 2H), 8.13 (d, 2H), 7.63 (d, 1H), 7.30-7.10 (m, 4H), 6.94 (d, 1H), 6.83 (dd, 1H), 6.28 (dd, 1H), 5.79 (d, 1H), 5.27 (q, 2H), 5.00-4.80 (m, 1H), 4.51-4.39 (m, 2H), 3.84-2.82 (m, 16H) 2.80 (s, 3H), 1.98 (s, 3H), 1.96 (s, 3H). MS (ESI) m/z 998.4 (M+H)⁺.

### Example 31

### (7R,16R)-10-({2-[(1 s,4s)-4-(carboxymethyl)cyclohexyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 31A

### methyl 2-(4-(4-(hydroxymethyl)pyrimidin-2-yl)cyclohex-3-en-1-yl)acetate

To a solution of methyl 2-(4-(4,4,5,5-tetramethyl-1,3,2-dioxaborolan-2-yl)cyclohex-3-en-1-yl)acetate (970 mg) and (2-chloropyrimidin-4-yl)methanol (500 mg) in tetrahydrofuran (14.7 mL) and saturated aqueous sodium bicarbonate (8.4 mL) was added tetrakis(triphenylphosphine)palladium (0) (400 mg). The reaction was purged with nitrogen and was heated to 75° C. overnight. The reaction was cooled, and diluted with ethyl acetate and water. The layers separated, and the aqueous layer was extracted with ethyl acetate three times. The combined organic layers were dried over anhydrous sodium sulfate, filtered and concentrated. The residue was purified by normal phase MPLC on a Teledyne Isco CombiFlash® Rf+40 g gold silica gel column eluting with 5-65% ethyl acetate in heptanes to give the title compound. ¹H NMR (400 MHz, dimethylsulfoxide-d₆) δ ppm 8.70 (d, 1H), 7.34 (d, 1H), 7.19-7.11 (m, 1H), 5.61-5.53 (m, 1H), 4.56-4.48 (m, 2H), 3.61 (s, 3H), 2.75-2.62 (m, 1H), 2.45-2.28 (m, 4H), 2.10-1.79 (m, 3H), 1.45-1.27 (m, 1H).

### Example 31B

### rel-methyl 2-((1s,4s)-4-(4-(hydroxymethyl)pyrimidin-2-yl)cyclohexyl)acetate

To a solution of Example 31A (450 mg) in tetrahydrofuran (4.5 mL) was added a Ra-Ni 2800, water slurry (430 mg) in a 20 mL Barnstead STEM RS10 reactor. The reactor was purged with argon, and the mixture was stirred at 1100 rpm under 50 psi of hydrogen at 25° C. After 48 hours, the reaction was vented, filtered and concentrated. The residue was purified by normal phase MPLC on a Teledyne Isco CombiFlash® Rf+40 g gold silica gel column eluting with 0-65% ethyl acetate in heptanes to give the title compound. ¹H NMR (500 MHz, dimethylsulfoxide-d₆) δ ppm 8.70 (d, 1H), 7.35 (d, 1H), 5.61-5.53 (m, 1H), 4.55-4.48 (m, 2H), 3.58 (s, 3H), 2.95-2.84 (m, 1H), 2.29 (d, 2H), 2.10-1.92 (m, 3H), 1.73-1.62 (m, 2H), 1.61-1.51 (m, 2H), 1.45-1.32 (m, 2H).

### Example 31C

### rel-methyl 2-((1r,4r)-4-(4-(hydroxymethyl)pyrimidin-2-yl)cyclohexyl)acetate

The title compound was also obtained during the synthesis described in Example 31B. ¹H NMR (500 MHz, dimethylsulfoxide-d₆) δ ppm 8.68 (d, 1H), 7.35 (d, 1H), 5.60-5.53 (m, 1H), 4.53-4.48 (m, 2H), 3.59 (s, 3H), 2.76-2.64 (m, 1H), 2.24 (d, 2H), 1.98-1.88 (m, 2H), 1.84-1.65 (m, 3H), 1.62-1.49 (m, 2H), 1.19-1.05 (m, 2H).

### Example 31D

### tert-butyl (7R,16R)-19,23-dichloro-1-(4-fluorophenyl)-10-({2-[(1s,4s)-4-(2-methoxy-2-oxoethyl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (30 mg), Example 31B (15 mg) in toluene (100 μL) and tetrahydrofuran (100 μL) was added triphenylphosphine (29 mg) followed by N,N,N′,N′-tetramethylazodicarboxamide (19 mg). The reaction was allowed to stir at 50° C. for three hours. The reaction was cooled, diluted with ethyl acetate, filtered over diatomaceous earth and concentrated. The residue was purified by normal phase MPLC on a Teledyne Isco CombiFlash® Rf+4 g gold silica gel column, eluting with 0-9% methanol in dichloromethane to give the title compound.

### Example 31E

### (7R,16R)-19,23-dichloro-1-(4-fluorophenyl)-10-({2-[(1s,4s)-4-(2-methoxy-2-oxoethyl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 31D (31 mg) in dichloromethane (150 μL) was added trifluoroacetic acid (150 μL), and the reaction was allowed to stir for 6 hours. The reaction was concentrated under a stream of nitrogen and the residue was taken up in water and acetonitrile. The mixture was purified by RP-HPLC on a Gilson PLC 2020 using a Luna™ column (250×50 mm, 10 mm) (5-80% over 30 minutes with acetonitrile in water containing 0.01% trifluoroacetic acid) to give the title compound.

### Example 31F

### (7R,16R)-10-({2-[(1s,4s)-4-(carboxymethyl)cyclohexyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 31E (27 mg) in tetrahydrofuran (375 μL) and methanol (375 μL) at room temperature was added a solution of lithium hydroxide (16 mg) in water (375 μL), and the reaction was allowed to sit overnight at 4° C. The reaction was quenched with trifluoroacetic acid (63 μL), taken up in dimethylsulfoxide and purified by RP-HPLC on a Gilson PLC 2020 using a Luna™ column (250×50 mm, 10 mm) (5-75% over 30 minutes with acetonitrile in water containing 0.01% trifluoroacetic acid) to give the title compound. ¹H NMR (500 MHz, dimethylsulfoxide-d₆) δ ppm 8.78-8.72 (m, 2H), 7.41 (d, 1H), 7.24-7.11 (m, 5H), 6.89 (d, 1H), 6.82 (dd, 1H), 6.25 (dd, 1H), 5.78 (d, 1H), 5.19-5.03 (m, 2H), 4.99-4.90 (m, 1H), 4.53-4.38 (m 2H), 3.64-3.54 (m, 1H), 3.41 (br s, 2H), 3.22 (br s, 2H), 3.16-2.77 (m, 9H), 2.19 (d, 2H), 2.08-1.90 (m, 8H), 1.76-1.65 (m, 2H), 1.64-1.53 (m, 2H), 1.47-1.34 (m, 2H). MS (ESI) m/z 983.2 (M−H)⁻.

### Example 32

### (7R,16R)-10-({2-[(1r,4r)-4-(carboxymethyl)cyclohexyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 32A

### tert-butyl (7R,16R)-19,23-dichloro-1-(4-fluorophenyl)-10-({2-[(1r,4r)-4-(2-methoxy-2-oxoethyl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (30 mg), Example 31C (15 mg) in toluene (100 μL) and tetrahydrofuran (100 μL) was added triphenylphosphine (29 mg) followed by N,N,N′,N′-tetramethylazodicarboxamide (19 mg), and the reaction was allowed to stir at 50° C. for 4 hours. Additional triphenylphosphine (29 mg) and N,N,N′,N′-tetramethylazodicarboxamide (19 mg) were added, and the reaction was heated for a further 2 hours before cooling to room temperature stirring overnight. The reaction diluted with ethyl acetate, filtered over diatomaceous earth and concentrated. The residue was purified by normal phase MPLC on a Teledyne Isco CombiFlash® Rf+4 g gold silica gel column eluting with 0.5-7.5% methanol in dichloromethane to give the title compound.

### Example 32B

### (7R,16R)-19,23-dichloro-1-(4-fluorophenyl)-10-({2-[(1r,4r)-4-(2-methoxy-2-oxoethyl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 32A (31 mg) in dichloromethane (150 μL) was added trifluoroacetic acid (150 μL), and the reaction was allowed to stir for 5 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-80% over 30 minutes with acetonitrile in water containing 0.01% trifluoroacetic acid) to give the title compound. ¹H NMR (500 MHz, dimethylsulfoxide-d₆) δ ppm 8.75 (s, 1H), 8.72 (d, 1H), 7.41 (d, 1H), 7.23-7.10 (m, 5H), 6.87 (d, 1H), 6.82 (dd, 1H), 6.24 (dd, 1H), 5.77 (d, 1H), 5.17-5.02 (m, 2H), 4.99-4.90 (m, 1H), 4.53-4.38 (m, 2H), 3.59 (s, 3H), 3.16-2.69 (m, 10H), 2.25 (d, 2H), 2.03-1.90 (m, 8H), 1.85-1.65 (m, 3H), 1.64-1.50 (m, 2H), 1.20-1.05 (m, 2H).

### Example 32C

### (7R,16R)-10-({2-[(1r,4r)-4-(carboxymethyl)cyclohexyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 32B (26 mg) in tetrahydrofuran (360 μL) and methanol (360 μL) at room temperature was added a solution of lithium hydroxide (16 mg) in water (360 μL). The reaction was allowed to sit overnight at 4° C. The reaction was quenched with trifluoroacetic acid (60 μL), taken up in dimethylsulfoxide and purified by RP-HPLC on a Gilson PLC 2020 using a Luna™ column (250×50 mm, 10 mm) (5-75% over 30 minutes with acetonitrile in water containing 0.01% trifluoroacetic acid) to give the title compound. ¹H NMR (500 MHz, dimethylsulfoxide-d₆) δ ppm 8.76 (s, 1H), 8.72 (d, 1H), 7.41 (d, 1H), 7.25-7.10 (5H), 6.91-6.78 (m, 2H), 6.24 (dd, 1H), 5.77 (d, 1H), 5.18-5.02 (m, 2H), 4.99-4.89 (m, 1H), 4.55-4.37 (m, 2H), 3.17-2.67 (m, 10H), 2.15 (d, 2H), 2.03-1.90 (m, 8H), 1.89-1.78 (m, 2H), 1.77-1.49 (m, 3H), 1.21-1.03 (m, 2H). MS (ESI) m/z 982.9 (M−H)⁻.

### Example 33

### (7R,16R)-19,23-dichloro-10-{[2-(6,6-difluoro-3-azabicyclo[3.1.0]hexan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 33A

### (2-(6,6-difluoro-3-azabicyclo[3.1.0]hexan-3-yl)pyrimidin-4-yl)methanol

A solution of 6,6-difluoro-3-azabicyclo[3.1.0]hexane, hydrochloric acid salt (270 mg), (2-chloropyrimidin-4-yl)methanol (210 mg) and N,N-diisopropylethylamine (810 μL) in acetonitrile (3.6 mL) was heated to 80° C. for 2 hours and at room temperature overnight. The reaction was diluted with water and extracted with ethyl acetate three times. The combined organic layers were washed with brine, dried over anhydrous sodium sulfate, filtered and concentrated. The residue was purified by normal phase MPLC on a Teledyne Isco CombiFlash® Rf+24 g gold silica gel column eluting with 0-65% ethyl acetate in dichloromethane to give the title compound. ¹H NMR (400 MHz, dimethylsulfoxide-d₆) δ ppm 8.32 (d, 1H), 6.75 (d, 1H), 5.45-5.33 (m, 1H), 4.44-4.30 (m, 2H), 3.97-3.82 (m, 2H), 3.79-3.63 (m, 2H), 2.71-2.55 (m, 2H).

### Example 33B

### tert-butyl (7R,16R)-19,23-dichloro-10-{[2-(6,6-difluoro-3-azabicyclo[3.1.0]hexan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (30 mg) and Example 33A (13 mg) in toluene (100 μL) and tetrahydrofuran (100 μL) was added triphenylphosphine (29 mg) followed by N,N,N′,N′-tetramethylazodicarboxamide (19 mg), and the reaction was allowed to stir at 50° C. for 4 hours. The reaction was cooled, diluted with ethyl acetate, and filtered over diatomaceous earth. The filtrate was concentrated. The residue was purified by normal phase MPLC on a Teledyne Isco CombiFlash® Rf+4 g gold silica gel column eluting with 0-8% methanol in dichloromethane to give the title compound. ¹H NMR (400 MHz, dimethylsulfoxide-d₆) δ ppm 8.74 (s, 1H), 8.36 (d, 1H), 7.28-7.10 (m, 5H), 6.91-6.73 (m, 3H), 6.03 (dd, 1H), 5.67 (d, 1H), 5.06-4.84 (m, 2H), 4.81-4.68 (m, 1H), 4.54-4.31 (m, 2H), 3.97-3.84 (m, 2H), 3.81-3.69 (m, 2H), 3.67-3.57 (m, 1H), 2.86 (d, 1H), 2.73-2.58 (m, 4H), 2.44-2.22 (m, 4H), 2.15 (s, 3H), 2.09 (s, 3H), 1.90 (s, 3H), 1.06 (s, 9H).

### Example 33C

### (7R,16R)-19,23-dichloro-10-{[2-(6,6-difluoro-3-azabicyclo[3.1.0]hexan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 33B (30 mg) in dichloromethane (150 μL) was added trifluoroacetic acid (150 μL), and the reaction was allowed to stir for 6 hours. The reaction mixture was concentrated under a stream of nitrogen and was taken up in water and acetonitrile. The mixture was purified by RP-HPLC on a Gilson PLC 2020 using a Luna™ column (250×50 mm, 10 mm) (5-80% over 30 minutes with acetonitrile in water containing 0.01% trifluoroacetic acid) to give the title compound. ¹H NMR (400 MHz, dimethylsulfoxide-d₆) δ ppm 8.75 (s, 1H), 8.33 (d, 1H), 7.26-7.09 (m, 5H), 6.88-6.71 (m, 3H), 6.28-6.18 (m, 1H), 5.82-5.73 (m, 1H), 5.05-4.85 (m, 3H), 4.55-4.36 (m, 2H), 3.95-3.84 (m, 2H), 3.47-2.77 (m, 12H), 2.70-2.59 (m, 2H), 2.03-1.92 (m, 6H). MS (ESI) m/z 960.0 (M−H)⁻.

### Example 34

### (7R,16R)-19,23-dichloro-10-{[2-(1,1-dioxo-1,2,3,6-tetrahydro-1λ⁶-thiopyran-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 34A

### 4-(4-(hydroxymethyl)pyrimidin-2-yl)-3,6-dihydro-2H-thiopyran 1,1-dioxide

To a mixture of (2-chloropyrimidin-4-yl)methanol (420 mg), 4-(4,4,5,5-tetramethyl-1,3,2-dioxaborolan-2-yl)-3,6-dihydro-2H-thiopyran 1,1-dioxide (750 mg) and Pd(amphos)Cl₂ (bis(di-tert-butyl (4-dimethylaminophenyl)phosphine)dichloropalladium (II), 411 mg) in a 20-mL vial was added a solution of potassium phosphate (2.5 g) in tetrahydrofuran (12 mL) and water (3.5 mL). The mixture was purged by bubbling nitrogen for 10 minutes, stirred at ambient temperature for 4 days and concentrated. The residue was purified by flash chromatography on a Teledyne Isco CombiFlash® system, eluting with 0-100% ethyl acetate in heptanes to provide the title compound. MS (APCI) m/z 241.3 (M+H)⁺.

### Example 34B

### tert-butyl (7R,16R)-19,23-dichloro-10-{[2-(1,1-dioxo-1,2,3,6-tetrahydro-1λ⁶-thiopyran-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 28E by replacing Example 12P and Example 28D with Example 16N and Example 34A, respectively. MS (APCI) m/z 1030.8 (M+H)⁺.

### Example 34C

### (7R,16R)-19,23-dichloro-10-{[2-(1,1-dioxo-1,2,3,6-tetrahydro-1λ⁶-thiopyran-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 28F by replacing Example 28E with Example 34B. ¹H NMR (400 MHz, dimethylsulfoxide-d₆) δ ppm 8.78-8.60 (m, 2H), 7.44 (d, 1H), 7.19-6.99 (m, 6H), 6.85-6.54 (m, 2H), 6.15 (dd, 1H), 5.74 (d, 1H), 5.18-4.98 (m, 2H), 4.80 (t, 1H), 4.37 (d, 2H), 3.95 (d, 3H), 3.11 (s, 7H), 2.71-2.60 (m, 3H), 2.14 (s, 3H), 1.91 (d, 6H), 1.17 (s, 9H).

### Example 35

### (7R,16R)-10-({2-[(4S*)-4-(carboxymethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 35A

### 2-(4-(4-(hydroxymethyl)pyrimidin-2-yl)cyclohex-3-en-1-yl)acetic Acid

To a solution of Example 31A (880 mg) in tetrahydrofuran (24 mL) and methanol (12 mL) at room temperature was added a solution of lithium hydroxide (400 mg) in water (12 mL), and the reaction was allowed to stir overnight. The reaction was diluted with water and extracted once with dichloromethane. The aqueous layer was acidified with aqueous hydrochloric acid (2 M) and extracted with ethyl acetate six times. The combined organic layers were dried over anhydrous sodium sulfate, filtered and concentrated to give the title compound that was used without further purification.

### Example 35B

### (S*)-tert-butyl 2-(4-(4-(hydroxymethyl)pyrimidin-2-yl)cyclohex-3-en-1-yl)acetate

To a solution of Example 35A (690 mg) in dichloromethane (6.9 mL) and tert-butanol (6.9 mL) was added ammonium chloride (445 mg), and the reaction was cooled to 0° C. 2-tert-Butyl-1,3-diisopropylisourea (1.7 g) was added, and the reaction was warmed to room temperature and stirred overnight. Additional ammonium chloride (445 mg) and 2-tert-butyl-1,3-diisopropylisourea (1.6 g) were added, and the reaction was stirred overnight. Additional ammonium chloride (445 mg) and 2-tert-butyl-1,3-diisopropylisourea (1.6 g) were added. The reaction was stirred for 5 hours. The reaction was diluted with saturated aqueous ammonium chloride and ethyl acetate. The mixture was filtered over diatomaceous earth, and the aqueous layer was extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 0-65% ethyl acetate in heptanes to give a mixture of enantiomers. The mixture was purified by chiral SFC using a Chiralpak AD-H column (30×250 mm, 5 micron) to give the title compound of arbitrarily assigned stereochemistry. Analytical SFC analysis using a Chiralpak AD-H column (5-50% methanol over 10 minutes) gave a retention time of 6.21 minutes. ¹H NMR (400 MHz, dimethylsulfoxide-d₆) δ ppm 8.70 (d, 1H), 7.34 (d, 1H), 7.23-7.09 (m, 1H), 5.57 (br s, 1H), 4.52 (d, 2H), 2.78-2.60 (m, 1H), 2.46-2.29 (m, 2H), 2.22 (d, 2H), 2.07-1.76 (m, 3H), 1.49-1.27 (m, 10H).

### Example 35C

### (R*)-tert-butyl 2-(4-(4-(hydroxymethyl)pyrimidin-2-yl)cyclohex-3-en-1-yl)acetate

The title compound was also obtained from the SFC separation of Example 35B. Analytical SFC analysis using a Chiralpak AD-H column (5-50% methanol over 10 minutes) gave a retention time of 4.13 minutes. ¹H NMR (400 MHz, dimethylsulfoxide-d₆) δ ppm 8.70 (d, 1H), 7.34 (d, 1H), 7.23-7.09 (m, 1H), 5.57 (br s, 1H), 4.52 (d, 2H), 2.78-2.60 (m, 1H), 2.46-2.29 (m, 2H), 2.22 (d, 2H), 2.07-1.76 (m, 3H), 1.49-1.27 (m, 10H).

### Example 35D

### tert-butyl (7R,16R)-10-({2-[(4S)-4-(2-tert-butoxy-2-oxoethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (30 mg) and Example 35B (17 mg) in toluene (100 μL) and tetrahydrofuran (100 μL) was added triphenylphosphine (29 mg) followed by N,N,N′,N′-tetramethylazodicarboxamide (19 mg), and the reaction was allowed to stir at 50° C. for 4 hours. The reaction was cooled, diluted with ethyl acetate, filtered over diatomaceous earth and concentrated. The residue was purified by normal phase MPLC on a Teledyne Isco CombiFlash® Rf+4 g gold silica gel column eluting with 0-7% methanol in dichloromethane to give the title compound. ¹H NMR (500 MHz, dimethylsulfoxide-d₆) δ ppm 8.77-8.72 (m, 2H), 7.38 (d, 1H), 7.26-7.13 (m, 5H), 6.89 (d, 1H), 6.82 (dd, 1H), 6.05 (dd, 1H), 5.67 (d, 1H), 5.20-5.01 (m, 2H), 4.79-4.68 (m, 1H), 4.52-4.36 (m, 2H), 3.67 (dd, 1H), 2.92-2.84 (m, 1H), 2.81-2.75 (m, 1H), 2.74-2.59 (m, 3H), 2.45-2.19 (m, 6H), 2.13 (s, 3H), 2.10 (s, 3H), 2.04-1.81 (m, 6H), 1.45-1.30 (m, 10H), 1.06 (s, 9H).

### Example 35E

### (7R,16R)-10-({2-[(4S*)-4-(carboxymethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 35D (37 mg) in dichloromethane (170 μL) was added trifluoroacetic acid (170 μL), and the reaction was allowed to stir for 6 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-75% over 30 minutes with acetonitrile in water containing 0.01% trifluoroacetic acid) to give the title compound. ¹H NMR (400 MHz, dimethylsulfoxide-d₆) δ ppm 8.76 (s, 1H), 8.73 (d, 1H), 7.37 (d, 1H), 7.28-7.08 (m, 5H), 6.87 (d, 1H), 6.81 (dd, 1H), 6.31-6.21 (m, 1H), 5.82-5.73 (m, 1H), 5.21-5.02 (m, 2H), 4.98-4.86 (m, 1H), 4.56-4.35 (m, 2H), 3.67-3.56 (m, 2H), 3.25-2.63 (m, 12H), 2.47-2.30 (m, 4H), 2.25 (d, 2H), 2.06-1.82 (m, 8H), 1.46-1.27 (m, 2H). MS (ESI) m/z 982.27 (M−H)⁻.

### Example 36

### (7R,16R)-10-({2-[(1R,5S,6r)-6-carboxy-3-azabicyclo[3,1,0]hexan-3-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 36A

### (1R,5S,6r)-ethyl 3-(4-(hydroxymethyl)pyrimidin-2-yl)-3-azabicyclo[3.1.0]hexane-6-carboxylate

A solution of (1R,5S,6r)-ethyl 3-azabicyclo[3.1.0]hexane-6-carboxylate, hydrochloric acid salt (320 mg), (2-chloropyrimidin-4-yl)methanol (200 mg) and N,N-diisopropylethylamine (790 μL) in acetonitrile (3.5 mL) was heated to 80° C. for 90 minutes and stirred at room temperature overnight. The reaction was diluted with water and extracted with ethyl acetate three times. The combined organic layers were washed with brine, dried over anhydrous sodium sulfate, filtered and concentrated. The residue was purified by normal phase MPLC on a Teledyne Isco CombiFlash® Rf+24 g gold silica gel column, eluting with 0-65% ethyl acetate in dichlormethane to give the title compound. ¹H NMR (500 MHz, dimethylsulfoxide-d₆) δ ppm 8.30 (d, 1H), 6.73 (d, 1H), 5.40-5.32 (m, 1H), 4.39-4.30 (m, 2H), 4.06 (q, 2H), 3.84 (d, 2H), 3.55-3.45 (m, 2H), 2.20-2.12 (m, 2H), 1.48-1.41 (m, 1H), 1.18 (t, 3H).

### Example 36B

### tert-butyl (7R,16R)-19,23-dichloro-10-({2-[(1R,5S,6r)-6-(ethoxycarbonyl)-3-azabicyclo[3.1.0]hexan-3-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (30 mg), Example 36A (10 mg) in tetrahydrofuran (100 μL) and toluene (100 μL) was added triphenylphosphine (29 mg) and N,N,N′,N′-tetramethylazodicarboxamide (19 mg), and the reaction was allowed to stir at 50° C. for 3 hours. The reaction was cooled, diluted with ethyl acetate, and filtered over diatomaceous earth. The filtrate was concentrated. The residue was purified by normal phase MPLC on a Teledyne Isco CombiFlash® Rf+4 g gold silica gel column eluting with 0-7.5% methanol in dichlormethane to give the title compound. ¹H NMR (400 MHz, dimethylsulfoxide-d₆) δ ppm 8.74 (s, 1H), 8.33 (d, 1H), 7.28-7.12 (m, 5H), 6.90-6.71 (m, 3H), 6.02 (dd, 1H), 5.67 (d, 1H), 5.03-4.83 (m, 2H), 4.81-4.68 (m, 1H), 4.53-4.34 (m, 2H), 4.06 (q, 2H), 3.90-3.78 (m, 2H), 3.62 (dd, 1H), 3.56-3.47 (m, 2H), 2.92-2.83 (m, 1H), 2.74-2.58 (m, 2H), 2.43-2.23 (m, 4H), 2.21-2.11 (m, 4H), 2.08 (s, 3H), 1.90 (s, 3H), 1.49-1.43 (m, 1H), 1.18 (t, 3H), 1.07 (s, 9H).

### Example 36C

### (7R,16R)-19,23-dichloro-10-({2-[(1R,5S,6r)-6-(ethoxycarbonyl)-3-azabicyclo[3.1.0]hexan-3-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 36B (31 mg) in dichloromethane (150 μL) was added trifluoroacetic acid (150 μL), and the reaction was allowed to stir for 5 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-85% over 30 minutes with acetonitrile in water containing 0.01% trifluoroacetic acid) to give the title compound. ¹H NMR (400 MHz, dimethylsulfoxide-d₆) δ ppm 8.75 (s, 1H), 8.31 (d, 1H), 7.25-7.09 (m, 5H), 6.87-6.70 (m, 3H), 6.23 (dd, 1H), 5.78 (d, 1H), 5.02-4.85 (m, 3H), 4.53-4.37 (m, 2H), 4.07 (q, 2H), 3.89-3.79 (m, 2H), 3.30-2.73 (m, 12H), 2.21-2.12 (m, 2H), 1.96 (s, 6H), 1.50-1.42 (m, 1H), 1.18 (t, 3H). MS (ESI) m/z 996.0 (M−H)⁻.

### Example 36D

### (7R,16R)-10-({2-[(1R,5S,6r)-6-carboxy-3-azabicyclo[3.1.0]hexan-3-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 36C (25 mg) in tetrahydrofuran (280 μL) and methanol (280 μL) at room temperature was added a solution of lithium hydroxide (12 mg) in water (280 μL), and the reaction was allowed to sit for 2 hours. The reaction was quenched with trifluoroacetic acid (50 μL), taken up in dimethylsulfoxide and purified by RP-HPLC on a Gilson PLC 2020 using a Luna™ column (250×50 mm, 10 mm) (5-75% over 30 minutes with acetonitrile in water containing 0.01% trifluoroacetic acid) to give the title compound. ¹H NMR (500 MHz, dimethylsulfoxide-d₆) δ ppm 8.75 (s, 1H), 8.30 (d, 1H), 7.23-7.09 (m, 5H), 6.86-6.76 (m, 2H), 6.73 (d, 1H), 6.27-6.19 (m, 1H), 5.82-5.75 (m, 1H), 5.01-4.85 (m, 3H), 4.52-4.36 (m, 2H), 3.88-3.79 (m, 2H), 3.18-2.76 (m, 10H), 2.18-2.08 (m, 2H), 1.97 (s, 6H), 1.37-1.31 (m, 1H). MS (ESI) m/z 968.0 (M−H)⁻.

### Example 37

### (7R,16R)-10-({2-[(4R*)-4-(carboxymethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 37A

### tert-butyl (7R,16R)-10-({2-[(4R)-4-(2-tert-butoxy-2-oxoethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (30 mg) and Example 35C (17 mg) in toluene (100 μL) and tetrahydrofuran (100 μL) was added triphenylphosphine (29 mg) followed by N,N,N′,N′-tetramethylazodicarboxamide (19 mg), and the reaction was allowed to stir at 50° C. for 4 hours. The reaction was cooled, diluted with ethyl acetate, filtered over diatomaceous earth and the filtrate was concentrated. The residue was purified by normal phase MPLC on a Teledyne Isco CombiFlash® Rf+4 g gold silica gel column eluting with 0-7% methanol in dichloromethane to give the title compound. ¹H NMR (400 MHz, dimethylsulfoxide-d₆) δ ppm 8.79-8.70 (m, 2H), 7.38 (d, 1H), 7.29-7.11 (m, 5H), 6.89 (d, 1H), 6.82 (dd, 1H), 6.05 (dd, 1H), 5.67 (d, 1H), 5.24-5.00 (m, 2H), 4.80-4.67 (m, 1H), 4.56-4.32 (m. 2H), 3.67 (dd, 1H), 2.93-2.83 (m, 1H), 2.76-2.58 (m, 3H), 2.46-2.18 (m, 8H), 2.13 (s, 3H), 2.10 (s, 3H), 2.05-1.81 (m, 6H), 1.41 (s, 9H), 1.06 (s, 9H).

### Example 37B

### (7R,16R)-10-({2-[(4R*)-4-(carboxymethyl)cyclohex-1-en-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 37A (34 mg) in dichloromethane (150 μL) was added trifluoroacetic acid (150 μL), and the reaction was allowed to stir for 5 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-80% over 30 minutes with acetonitrile in water containing 0.01% trifluoroacetic acid) to give the title compound. ¹H NMR (400 MHz, dimethylsulfoxide-d₆) δ ppm 8.76 (s, 1H), 8.72 (d, 1H), 7.37 (d, 1H), 7.27-7.10 (m, 5H), 6.86 (d, 1H), 6.81 (dd, 1H), 6.29-6.21 (m, 1H), 5.80-5.75 (m, 1H), 5.20-5.03 (m, 2H), 4.99-4.87 (m, 1H), 4.55-4.36 (m, 2H), 3.16-2.64 (m, 8H), 2.46-2.29 (m, 2H), 2.25 (d, 2H), 2.07-1.80 (m, 8H), 1.46-1.27 (m, 1H). MS (ESI) m/z 980.9 (M−H)⁻.

### Example 38

### (7R,16R)-19,23-dichloro-10-({2-[(1S,2S)-1,2-dihydroxycyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 38A

### 4-(((tert-butyldimethylsilyl)oxy)methyl)-2-chloropyrimidine

(2-Chloropyrimidin-4-yl)methanol (3.0 g), triethylamine (5.79 mL), and 4-dimethylaminopyridine (0.25 g) were dissolved in dichloromethane (104 mL). The reaction mixture was cooled by an ice-bath, tert-Butylchlorodimethylsilane (3.28 g) was added in portions and the reaction mixture was stirred in the water bath overnight. The reaction mixture was partitioned between dichloromethane and water. The organic layer was washed with saturated aqueous sodium bicarbonate solution, aqueous hydrochloric acid (2M), once more with saturated aqueous sodium bicarbonate solution, dried by a TPS cartridge, and concentrated. Purification was performed on a silica gel column (80 g, 0-11% methanol in dichloromethane). The desired fractions were combined and the solvents were removed under reduced pressure to provide the title compound. MS (ESI) m/z 259.1 (M+H)⁺.

### Example 38B

### 4-(((tert-butyldimethylsilyl)oxy)methyl)-2-(cyclohex-1-en-1-yl)pyrimidine

Example 38A (8 g), cyclohex-1-en-1-ylboronic acid (4.67 g), PdCl₂ (dppf)-dichloromethane complex (1.262 g) and sodium carbonate (61.8 mL) were taken up in 80 mL dioxane, subjected to several vacuum/nitrogen cycles, then heated to 80° C. overnight. The reaction was cooled, poured into ethyl acetate, washed with water and brine, dried over sodium sulfate, filtered and concentrated. The crude material was chromatographed on silica gel using 1% ethyl acetate in heptanes as eluent to give the title compound. ¹H NMR (400 MHz, dimethylsulfoxide-d₆) δ ppm 8.72 (d, 1H), 7.27 (d, 1H), 7.21 (dd, 1H), 4.69 (s, 2H), 2.46 (m, 2H), 2.23 (m, 2H), 1.68 (m, 2H), 1.59 (m, 2H), 0.91 (s, 9H), 0.10 (s, 6H). MS (ESI) m/z 305.2 (M+H)⁺.

### Example 38C

### (1S,2S)-1-(4-(((tert-butyldimethylsilyl)oxy)methyl)pyrimidin-2-yl)cyclohexane-1,2-diol

AD-Mix-(7 g, 1.4 g/mmol) and methanesulfonamide (0.476 g) were taken up in 25 mL tert-butanol and 25 mL water, cooled to 0° C. and Example 38B (1.523 g, 5 mmol) was added. The mixture was allowed to warm to room temperature overnight. Additional AD-Mix-(7 g) was added, and the reaction was stirred at 50° C. overnight. The mixture was cooled and sodium sulfite was added and the mixture was stirred for 1 hour. The reaction was cooled, poured into ethyl acetate, and washed with 1M aqueous sodium hydroxide solution, water and brine. The organic layer was dried over sodium sulfate, filtered and concentrated. The crude material was chromatographed on silica gel using 2-20% ethyl acetate in heptanes as eluent to give the title compound. ¹H NMR (400 MHz, dimethylsulfoxide-d₆) δ ppm 8.79 (d, 1H), 7.38 (d, 1H), 4.75 (s, 2H), 4.65 (s, 1H), 4.13 (d, 1H), 3.82 (m, 1H), 1.91 (ddd, 1H), 1.68 (dd, 1H), 1.61 (m, 2H), 1.52 (m, 2H), 1.44 (m, 1H), 1.33 (m, 1H), 0.92 (s, 9H), 0.11 (s, 6H). MS (ESI) m/z 339.1 (M+H)⁺.

### Example 38D

### (1S,2S)-1-(4-(hydroxymethyl)pyrimidin-2-yl)cyclohexane-1,2-diol

Tetra-N-butylammonium fluoride (3.57 mL, 1M in tetrahydrofuran) was added to Example 38C (1.1 g) in 40 mL tetrahydrofuran, and the reaction was stirred for 30 minutes, poured into ethyl acetate, washed with water and brine, dried over sodium sulfate, filtered and concentrated. The crude material was chromatographed on silica gel using 0-5% methanol in ethyl acetate as the eluent to give the title compound. ¹H NMR (400 MHz, dimethylsulfoxide-d₆) δ ppm 8.77 (d, 1H), 7.43 (d, 1H), 5.65 (t, 1H), 4.71 (s, 1H), 4.57 (d, 2H), 4.11 (d, 1H), 3.83 (m, 1H), 1.90 (ddd, 1H), 1.69 (dd, 1H), 1.63 (m, 2H), 1.56 (m, 2H), 1.43 (m, 1H), 1.35 (m, 1H). MS (ESI) m/z 225.1 (M+H)⁺.

### Example 38E

### (7R,16R)-19,23-dichloro-10-({2-[(1S,2S)-1,2-dihydroxycyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 16N (50 mg), Example 38D (20.77 mg), triphenylphosphine (48.6 mg) and N,N,N′,N′-tetramethylazodicarboxylate (31.9 mg) were stirred in 0.5 mL tetrahydrofuran and 0.5 mL toluene at 50° C. for 1 hour. The crude material was chromatographed on silica gel using 0-10% methanol in dichloromethane to give the coupled ester. The material was taken up in 10 mL 1:1 dichloromethane/trifluoroacetic acid, and the solution was stirred overnight, and concentrated. The crude material was taken up in 2 mL methanol and dimethylformamide, and purified by reverse phase chromatography using a 30-75% gradient of acetonitrile in water (with 0.1% ammonium acetate) over 30 minutes on a Grace Reveleris equipped with a Luna™ column: C18(2), 100 Å, 250×50 mm. The fractions containing the desired compound were combined, frozen and lyophilized to isolate the title compound. ¹H NMR (400 MHz, dimethylsulfoxide-d₆) δ ppm 8.73 (d, 1H), 8.66 (s, 1H), 7.44 (dd, 1H), 7.09 (m, 4H), 6.80 (dd, 1H), 6.72 (dd, 1H), 6.17 (d, 1H), 5.74 (d, 1H), 5.07 (dd, 2H), 4.82 (m, 1H), 4.67 (d, 1H), 4.51 (s, 1H), 4.38 (m, 2H), 4.10 (m, 1H), 3.79 (m, 2H), 3.27 (m, 4H), 2.89 (dd, 2H), 2.64 (m, 4H), 2.35 (s, 3H), 1.92 (s, 3H), 1.89 (s, 3H), 1.85 (m, 2H), 1.59 (m, 4H), 1.40 (m, 1H), 1.35 (m, 1H). MS (ESI) m/z 959.2 (M+H)⁺.

### Example 39

### (7R,16R)-19,23-dichloro-10-({2-[(1R,2R)-1,2-dihydroxycyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 39A

### (1R,2R)-1-(4-(((tert-butyldimethylsilyl)oxy)methyl)pyrimidin-2-yl)cyclohexane-1,2-diol

The title compound was prepared by substituting AD-Mix- for AD-Mix- in Example 38C. ¹H NMR (400 MHz, dimethylsulfoxide-d₆) δ ppm 8.79 (d, 1H), 7.38 (d, 1H), 4.76 (s, 2H), 4.64 (s, 1H), 4.10 (d, 1H), 3.80 (m, 1H), 1.91 (ddd, 1H), 1.67 (dd, 1H), 1.62 (m, 2H), 1.52 (m, 2H), 1.45 (m, 1H), 1.34 (m, 1H), 0.92 (s, 9H), 0.11 (s, 6H). MS (ESI) m/z 339.1 (M+H)⁺.

### Example 39B

### (1R,2R)-1-(4-(hydroxymethyl)pyrimidin-2-yl)cyclohexane-1,2-diol

The title compound was prepared by substituting Example 39A for Example 38C in Example 38D. ¹H NMR (400 MHz, dimethylsulfoxide-d₆) δ ppm 8.77 (d, 1H), 7.44 (d, 1H), 5.65 (t, 1H), 4.72 (s, 1H), 4.58 (d, 2H), 4.10 (d, 1H), 3.82 (m, 1H), 1.92 (ddd, 1H), 1.69 (dd, 1H), 1.63 (m, 2H), 1.53 (m, 2H), 1.46 (m, 1H), 1.32 (m, 1H). MS (ESI) m/z 225.1 (M+H)⁺.

### Example 39C

### (7R,16R)-19,23-dichloro-10-({2-[(1R,2R)-1,2-dihydroxycyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was prepared by substituting Example 39B for Example 38D in Example 38E. ¹H NMR (400 MHz, dimethylsulfoxide-d₆) δ ppm 8.74 (d, 1H), 8.65 (s, 1H), 7.46 (dd, 1H), 7.08 (m, 4H), 6.80 (dd, 1H), 6.68 (dd, 1H), 6.14 (d, 1H), 5.78 (d, 1H), 5.08 (dd, 2H), 4.83 (m, 1H), 4.65 (d, 1H), 4.59 (s, 1H), 4.38 (m, 2H), 4.10 (m, 1H), 3.78 (m, 2H), 3.20 (m, 4H), 2.87 (dd, 2H), 2.62 (m, 4H), 2.19 (s, 3H), 1.90 (s, 6H), 1.85 (m, 2H), 1.55 (m, 4H), 1.39 (m, 1H), 1.28 (m, 1H). MS (ESI) m/z 959.2 (M+H)⁺.

### Example 40

### (7R,16R)-19,23-dichloro-10-{[2-(1,1-dioxo-1λ⁶-thian-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 40A

### 4-(4-(hydroxymethyl)pyrimidin-2-yl)tetrahydro-2H-thiopyran 1,1-dioxide

Example 34A (138 mg) in tetrahydrofuran (2 mL) was added to a Raney®-Nickel 2800/water slurry (140 mg) in a 20 mL Barnstead Hast C. The mixture was stirred for 24 hours under 50 psi hydrogen at 25° C. and was filtered. The filtrate was concentrated and the residue was purified by flash chromatography on a Teledyne Isco CombiFlash® system, eluting with ethyl acetate to provide the title compound.

### Example 40B

### tert-butyl (7R,16R)-19,23-dichloro-10-{[2-(1,1-dioxo-1λ⁶-thian-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 28E by replacing Example 12P and Example 28D with Example 16N and Example 40A, respectively.

### Example 40C

### (7R,16R)-19,23-dichloro-10-{[2-(1,1-dioxo-1λ⁶-thian-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 28F by replacing Example 28E with Example 40B. ¹H NMR (501 MHz, dimethylsulfoxide-d₆) δ ppm 8.78-8.60 (m, 2H), 7.42 (d, 1H), 7.17-7.10 (m, 2H), 7.10-7.04 (m, 2H), 6.81 (d, 1H), 6.70 (dd, 1H), 6.17 (dd, 1H), 5.72 (d, 1H), 5.17-4.93 (m, 2H), 4.81 (p, 1H), 4.38 (d, 2H), 3.55 (dd, 1H), 3.18 (dq, 1H), 3.10-2.99 (m, 2H), 2.89 (dd, 1H), 2.64 (qd, 2H), 2.28-2.16 (m, 7H), 1.90 (d, 6H).

### Example 41

### (7R,16R)-10-({2-[4-(carboxymethyl)-4-methylpiperidin-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 41A

### ethyl 2-(1-(4-(hydroxymethyl)pyrimidin-2-yl)-4-methylpiperidin-4-yl)acetate

A solution of ethyl 2-(4-methylpiperidin-4-yl)acetate, hydrochloric acid salt (320 mg), (2-chloropyrimidin-4-yl)methanol (175 mg) and N,N-diisopropylethylamine (680 μL) in acetonitrile (3 mL) was heated to 80° C. for 2 hours and stirred at room temperature overnight. The reaction was diluted with water and extracted with ethyl acetate three times. The combined organic layers were washed with brine, dried over anhydrous sodium sulfate, filtered and concentrated. The residue was purified by normal phase MPLC on a Teledyne Isco CombiFlash® Rf+24 g gold silica gel column eluting with 0-40% ethyl acetate in dichloromethane. The desired containing fractions were concentrated, and the residue was purified by RP-HPLC on a Gilson PLC 2020 using a Luna™ column (250×50 mm, 10 mm) (5-65% over 30 minutes with acetonitrile in water containing 0.01% trifluoroacetic acid). The desired containing fractions were combined, washed with saturated aqueous sodium bicarbonate and extracted with dichloromethane three times. The organic layers were dried over anhydrous sodium sulfate, filtered and concentrated to give the title compound. ¹H NMR (400 MHz, dimethylsulfoxide-d₆) δ ppm 8.29 (d, 1H), 6.66 (d, 1H), 5.41-5.31 (m, 1H), 4.33 (d, 2H), 4.05 (q, 2H), 3.92-3.77 (m, 2H), 3.68-3.51 (m, 2H), 2.30 (s, 2H), 1.57-1.44 (m, 2H), 1.43-1.31 (m, 2H), 1.17 (t, 3H), 1.05 (s, 3H).

### Example 41B

### tert-butyl (7R,16R)-19,23-dichloro-10-({2-[4-(2-ethoxy-2-oxoethyl)-4-methylpiperidin-1-yl]pyrimidin-4-yl}methoxy)-1-(4-fluorophenyl)-20,22-dimethyl-16-[(4-methylpiperazin-1-yl)methyl]-7,8,15,6-tetrahydro-18,21-etheno-13,9-(metheno)-6,14,17-trioxa-2-thia-3,5-diazacyclononadeca[1,2,3-cd]indene-7-carboxylate

To a vial containing Example 16N (30 mg) and Example 41A (16 mg) in toluene (100 μL) and tetrahydrofuran 100 μL was added triphenylphosphine (29 mg) followed by N,N,N′,N′-tetramethylazodicarboxamide (19 mg), and the reaction was allowed to stir at 50° C. for two hours. The reaction was cooled, diluted with ethyl acetate, filtered over diatomaceous earth and the filtrate was concentrated. The residue was purified by normal phase MPLC on a Teledyne Isco CombiFlash® Rf+24 g gold silica gel column eluting with 0-7% methanol in dichloromethane to give the title compound.

### Example 41C

### (7R,16R)-19,23-dichloro-10-({2-[4-(2-ethoxy-2-oxoethyl)-4-methylpiperidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 41B (36 mg) in dichloromethane (170 μL) was added trifluoroacetic acid (170 μL), and the reaction was allowed to stir for 5 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-75% over 30 minutes with acetonitrile in water containing 0.01% trifluoroacetic acid) to give the title compound. ¹H NMR (400 MHz, dimethylsulfoxide-d₆) δ ppm 8.75 (s, 1H), 8.31 (d, 1H), 7.26-7.09 (m, 5H), 6.88-6.76 (m, 2H), 6.66 (d, 1H), 6.28-6.219 (m, 1H), 5.81-5.73 (m, 2H), 5.02-4.83 (m, 3H), 4.56-4.35 (m, 2H), 4.05 (q, 2H), 3.94-3.82 (m, 2H), 3.69-3.45 (m, 4H), 3.24-2.74 (m, 10H), 2.31 (s, 2H), 1.97 (s, 3H), 1.96 (s, 3H), 1.58-1.45 (m, 2H), 1.43-1.33 (m, 2H), 1.17 (t, 3H), 1.06 (s, 3H). MS (ESI) m/z 1026.1 (M−H)⁻.

### Example 41D

### (7R,16R)-10-({2-[4-(carboxymethyl)-4-methylpiperidin-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 41C (23 mg) in tetrahydrofuran (250 μL) and methanol (250 μL) at room temperature was added a solution of lithium hydroxide (11 mg) in water (250 μL), and the reaction was stirred for 4 hours. The reaction was quenched with trifluoroacetic acid (45 μL), taken up in dimethylsulfoxide and purified by RP-HPLC on a Gilson PLC 2020 using a Luna™ column (250×50 mm, 10 mm) (5-80% over 30 minutes with acetonitrile in water containing 10 mM ammonium acetate) to give the title compound. ¹H NMR (400 MHz, dimethylsulfoxide-d₆) δ ppm 8.73 (s, 1H), 8.29 (d, 1H), 7.25-7.07 (m, 5H), 6.80 (d, 1H), 6.72 (dd, 1H), 6.67 (d, 1H), 6.25-6.17 (m, 1H), 5.85-5.78 (m. 1H), 5.00-4.80 (m, 3H), 4.51-4.36 (m, 2H), 3.93-3.80 (m, 2H), 3.68-3.52 (m, 2H), 2.99-2.87 (m, 2H), 2.76-2.59 (m, 2H), 2.30-2.18 (m, 5H), 1.96 (s, 6H), 1.60-1.47 (m, 2H), 1.45-1.34 (m, 2H), 1.33-1.19 (m, 2H), 1.06 (s, 3H). MS (ESI) m/z 998.1 (M−H)⁻.

### Example 42

### (7R,16R)-19,23-dichloro-1-(4-fluorophenyl)-20,22-dimethyl-16-[(4-methylpiperazin-1-yl)methyl]-10-{[2-(2-oxa-6-azaspiro[3.3]heptan-6-yl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 42A

### methyl 2-(2-oxa-6-azaspiro[3.3]heptan-6-yl)pyrimidin-4-carboxylate

To a solution of 2-oxa-6-azaspiro[3.3]heptane hemioxalate (1.04 g) in dioxane (10 mL) was added triethylamine (1.55 mL) and the reaction mixture was stirred for 10 minutes at ambient temperature. Methyl 2-chloropyrimidine-4-carboxylate (500 mg) was added and the reaction mixture was stirred at 80° C. for 6 hours in a Biotage® Initiator microwave unit. To the reaction mixture was added water and the aqueous phase was extracted twice with ethyl acetate. The combined organic extracts were washed with brine, dried with sodium sulfate, filtered, and concentrated in vacuo. The crude product was used without any further purification in the next step. MS (ESI) m/z 230.4 (M+H)⁺.

### Example 42B

### (2-(2-oxa-6-azaspiro[3.3]heptan-6-yl)pyrimidin-4-yl)methanol

To a solution of Example 42A (500 mg) in methanol (15 mL) was added NaBH₄ (121 mg) at 0° C. and the reaction mixture was stirred for 4 hours at ambient temperature. The reaction mixture was concentrated in vacuo. To the residue was added water and the aqueous phase was extracted three times with dichloromethane. The combined organic extracts were washed with brine, dried via DryDisk® and concentrated in vacuo. The crude product was used without any further purification in the next step. MS (APCI) m/z 208.2 (M+H)⁺.

### Example 42C

### (2-(2-oxa-6-azaspiro[3.3]heptan-6-yl)pyrimidin-4-yl)methyl methanesulfonate

Example 42B (99 mg) was dissolved in dichloromethane (4.5 mL) under a nitrogen atmosphere and cooled to 0° C. with ice water. Triethylamine (190 μL) and methanesulfonyl chloride (46 μL) were added and the reaction mixture was stirred with cooling for 1 hour. Brine was added to the reaction mixture and the aqueous layer was extracted with dichloromethane. The combined organic extracts were dried over anhydrous magnesium sulfate, filtrated and concentrated in vacuo. The crude product was used without any further purification in the next step. MS (APCI) m/z 286.2 (M+H)⁺.

### Example 42D

### tert-butyl (7R,16R)-19,23-dichloro-1-(4-fluorophenyl)-20,22-dimethyl-16-[(4-methylpiperazin-1-yl)methyl]-10-{[2-(2-oxa-6-azaspiro[3.3]heptan-6-yl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylate

A 4 mL vial, equipped with stir bar, was charged with Example 16N (100 mg) and Example 42C (63.4 mg). Dimethylformamide (412 μL) and subsequently cesium carbonate (121 mg) were added. The reaction mixture was stirred at ambient temperature for 150 minutes. The reaction mixture was added to cold aqueous sodium bicarbonate solution (5%). The precipitate was filtered off after 5 minutes and washed twice with cold water. The precipitate was dried in vacuo overnight at 30° C. to provide the title compound. MS (ESI) m/z 998.4 (M+H)⁺.

### Example 42E

### (7R,16R)-19,23-dichloro-1-(4-fluorophenyl)-20,22-dimethyl-16-[(4-methylpiperazin-1-yl)methyl]-10-{[2-(2-oxa-6-azaspiro[3.3]heptan-6-yl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylic Acid

To a solution of Example 42D (49.5 mg) in dichloromethane (330 μL) was added trifluoroacetic acid (382 μL). The reaction mixture was stirred for 135 minutes at ambient temperature. The reaction mixture was then concentrated in vacuo. The residue was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.74 (d, 1H), 8.31 (d, 1H), 7.20 (m, 2H), 7.14 (m, 2H), 6.81 (d, 1H), 6.77 (d, 1H), 6.74 (m, 1H), 6.20 (m, 1H), 5.78 (s, 1H), 4.92 (m, 1H), 4.88 (m, 2H), 4.71 (s, 4H), 4.44 (m, 2H), 4.19 (s, 4H), 3.57 (m, 1H), 2.93 (m, 1H), 2.68 (m, 2H), 2.55-2.25 (m, 8H), 2.19 (s, 3H), 1.99 (s, 3H), 1.97 (s, 3H). MS (ESI) m/z 942.2 (M+H)⁺.

### Example 43

### (7R,16R)-19-chloro-1-(4-fluorophenyl)-20-methyl-16-[(4-methylpiperazin-1-yl)methyl]-10-({2-[(2R)-1-(23-oxo-2,5,8,11,14,17,20-heptaoxatricosan-23-yl) pyrrolidin-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 43A

### (R)-benzyl 2-carbamoylpyrrolidine-1-carboxylate

To a solution of (R)-1-((benzyloxy) carbonyl) pyrrolidine-2-carboxylic acid (25 g) in tetrahydrofuran (250 mL) was added carbonyldiimidazole (48.8 g) with stirring at 15° C. for 2 hours. Ammonium hydroxide (200 mL) was added to the reaction and stirring was continued at 0° C. for 2 hours. The mixture was poured into a separatory funnel and the layers were separated. The aqueous layer was extracted five times with dichloromethane and the combined organic phase was dried over sodium sulfate, filtered and concentrated. The crude product was purified by column chromatography on silica gel, eluting with 1-2.5% methanol in dichloromethane to give the title compound.

### Example 43B

### (R)-benzyl 2-(imino(methoxy)methyl) pyrrolidine-1-carboxylate

To a solution of Example 43A (27 g) in dichloromethane (500 mL) was added trimethyloxonium tetrafluoroborate (29.0 g) at 0° C. and the reaction was stirred at 25° C. for 2 hours. The reaction was quenched with saturated aqueous sodium bicarbonate solution (200 mL) and extracted twice with dichloromethane. The combined organic phase was dried over sodium sulfate, filtered and concentrated. The crude product was purified by column chromatography on silica gel, eluting with 1-20% methanol in dichloromethane to give the title compound. ¹H NMR (400 MHz, chloroform-d) δ ppm 7.19-7.27 (m, 5H), 5.00-5.09 (m, 2H), 4.13-4.34 (m, 1H), 4.13-4.34 (m, 1H), 3.57-3.73 (m, 3H), 3.39-3.51 (m, 2H), 1.94-2.08 (m, 1H), 1.84-1.92 (m, 1H), 1.67-1.81 (m, 2H).

### Example 43C

### (R)-benzyl 2-carbamimidoylpyrrolidine-1-carboxylate

To a solution of Example 43B (18 g) in methanol (300 mL) was added ammonium chloride (7.34 g) at 10° C. The reaction was stirred at 80° C. for 12 hours. The mixture was concentrated to give the crude product which was washed with dichloromethane and filtered. The filtrate was concentrated under reduced pressure to give the title compound. ¹H NMR (400 MHz, dimethylsulfoxide-d₆) δ ppm 9.08 (br s, 2H), 7.41-7.29 (m, 5H), 6.59 (br s, 1H), 5.16-5.01 (m, 2H), 3.62-3.53 (m, 1H), 3.49-3.31 (m, 2H), 2.43-2.20 (m, 1H), 1.98-1.60 (m, 3H).

### Example 43D

### (R)-benzyl 2-(4-(dimethoxymethyl)pyrimidin-2-yl) pyrrolidine-1-carboxylate

To a solution of Example 43C (28 g) in methanol (200 mL) was added (E)-4-(dimethylamino)-1,1-dimethoxybut-3-en-2-one (29.4 g) in one portion with stirring at 80° C. for 12 hours. The reaction was cooled to 20° C. and concentrated under reduced pressure. The crude product was purified by column chromatography on silica gel, eluting with 1-3% ethyl acetate in petroleum ether to give the title compound. ¹H NMR (400 MHz, chloroform-d) δ ppm 8.59-8.78 (m, 1H), 7.29-7.45 (m, 3H), 7.18 (br d, 2H), 6.96 (br d, 1H), 5.10-5.18 (m, 2H), 4.98-5.06 (m, 1H), 4.84-4.93 (m, 1H), 3.61-3.89 (m, 2H), 3.31-3.46 (m, 6H), 2.32-2.55 (m, 1H), 2.01-2.08 (m, 2H), 1.87-1.97 (m, 1H).

### Example 43E

### (R)-benzyl 2-(4-(hydroxymethyl)pyrimidin-2-yl) pyrrolidine-1-carboxylate

To a solution of Example 43D (18 g) in 1,4-dioxane (250 mL) was added 4M aqueous hydrochloric acid (250 mL) in portions at 15° C. The mixture was stirred at 60° C. for 12 hours. The reaction mixture was cooled to 0° C. and NaOH (31.2 g) was added portionwise at 0° C. The PH of the reaction mixture was adjusted to 8 using 10% aqueous K₂CO₃ solution. To the reaction mixture was then added sodium borohydride (3.75 g) in portions with stirring for 2 hours at 0° C. The reaction mixture was extracted twice with ethyl acetate. The combined organic layers were washed with brine (200 mL), dried over sodium sulfate, filtered and concentrated to give the crude product which was purified by prep-SFC on a Thar SFC80 preparative SFC using a Chiralpak IC-H 250*30 mm i.d. 5 m column, Mobile phase: A for carbon dioxide and B for methanol (0.1% ammonium hydroxide). Gradient: B %=35%. Flow rate: 65 g/minute. ¹H NMR (400 MHz, chloroform-d) δ ppm 8.59-8.41 (m, 1H), 7.28 (br s, 1H), 7.27-7.20 (m, 1H), 7.19-7.07 (m, 2H), 7.06-6.94 (m, 1H), 6.88 (br d, 1H), 5.10-4.95 (m, 2H), 4.76 (d, 1H), 4.63 (br d, 1H), 4.51 (br d, 1H), 3.78-3.67 (m, 1H), 3.66-3.53 (m, 1H), 3.50-3.19 (m, 1H), 2.44-2.25 (m, 1H), 1.95 (br d, 2H), 1.89-1.78 (m, 1H).

### Example 43F

### (R)-(2-(pyrrolidin-2-yl)pyrimidin-4-yl)methanol

Example 43E (429 mg) in 6.25 mL 6N aqueous hydrochloric acid was heated under reflux for 75 minutes. The solution was cooled, and extracted with 7.5 mL ether. The aqueous solution was cooled in an ice bath, then 7.5 mL 5.0 N aqueous NaOH was added dropwise (final pH>10). The mixture was extracted with 5×10 mL dichloromethane. The combined extracts were dried over sodium sulfate, filtered and concentrated to give the title compound. ¹H NMR (400 MHz, dimethylsulfoxide-d₆) δ ppm 8.71 (d, 1H), 7.41 (d, 1H), 4.54 (s, 2H), 4.13 (t, 1H), 3.55 (m, 2H), 3.09 (m, 1H), 2.80 (m, 1H), 2.13 (m, 1H), 1.72 (m, 3H). MS (ESI) m/z 180.0 (M+H)⁺.

### Example 43G

### (R)-23-(2-(4-(hydroxymethyl)pyrimidin-2-yl) pyrrolidin-1-yl)-2,5,8,11,14,17,20-heptaoxatricosan-23-one

To 2,5,8,11,14,17,20-heptaoxatricosan-23-oic acid (260 mg) in N,N-dimethylformamide (2.5 mL), was added O-(7-azabenzotriazol-1-yl)-N,N,N′,N′-tetramethyluronium hexafluorophosphate (247 mg) and N-ethyl-N-isopropylpropan-2-amine (370 μL). The reaction was stirred for 3 minutes, and added to a solution of Example 43F (90 mg) and N-ethyl-N-isopropylpropan-2-amine (240 μL) in dimethylformamide (2.5 mL). The combined mixture was stirred for 24 hours. The mixture was diluted with 4 mL dimethylformamide/water 1/1, then chromatographed on a Grace Revelris system using a Luna™ 250×50 mm column, 5-60% acetonitrile in 0.1% aqueous trifluoroacetic acid over 30 minutes to give the title compound. MS (ESI) m/z 530.0 (M+H)⁺.

### Example 43H

### (R)-(2-(1-(2,5,8,11,14,17,20-heptaoxatricosan-23-oyl) pyrrolidin-2-yl)pyrimidin-4-yl)methyl methanesulfonate

To Example 43G (530 mg) in dichloromethane (5 mL) cooled in an ice-water bath was added triethylamine (290 μL). The reaction was stirred for 15 minutes, and methanesulfonyl chloride (160 μL) was added dropwise. The reaction was stirred at room temperature for 1 hour. Sodium carbonate solution (5 mL, 2M) was added, the reaction was stirred for 15 minutes, and extracted twice with dichloromethane. The combined organic layers were dried over sodium sulfate, filtered and concentrated to give the title compound which was used in the next step without further purification. MS (ESI) m/z 608.1 (M+H)⁺.

### Example 431

### tert-butyl (7R,16R)-19-chloro-1-(4-fluorophenyl)-20-methyl-16-[(4-methylpiperazin-1-yl)methyl]-10-({2-[(2R)-1-(23-oxo-2,5,8,11,14,17,20-heptaoxatricosan-23-yl) pyrrolidin-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

To Example 43H (608 mg) in dimethylformamide (1.0 mL) was added Example 12P (157 mg), followed by cesium carbonate (170 mg) and the reaction was stirred for 24 hours. The mixture was diluted with 4 mL dimethylformamide, then chromatographed on a Grace Revelris system using a Luna™ 250×50 mm column, 20-80% acetonitrile in 0.1% aqueous trifluoroacetic acid over 30 minutes to give the title compound. MS (ESI) m/z 1272.6 (M+H)⁺.

### Example 43J

### (7R,16R)-19-chloro-1-(4-fluorophenyl)-20-methyl-16-[(4-methylpiperazin-1-yl)methyl]-10-({2-[(2R)-1-(23-oxo-2,5,8,11,14,17,20-heptaoxatricosan-23-yl) pyrrolidin-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

To Example 431 (58 mg) in dichloromethane (0.5 mL) was added trifluoroacetic acid (0.5 mL), and the reaction was stirred for 4 hours. The mixture was concentrated and taken up in 2 mL dimethylformamide and 0.5 mL water, then chromatographed on a Grace Revelris system using a Luna™ 250×50 mm column, 5-75% acetonitrile in 10 mM ammonium acetate over 30 minutes to give the title compound. ¹H NMR (400 MHz, dimethylsulfoxide-d₆) δ ppm 8.81 (d, 1H), 8.53 (s, 1H), 7.48 (dd, 1H), 7.04 (m, 4H), 6.97 (dd, 1H), 6.84 (dd, 1H), 6.51 (s, 2H), 6.17 (d, 1H), 5.80 (m, 1H), 5.33 (s, 2H), 4.96 (m, 1H), 4.75 (m, 1H), 4.66 (m, 2H), 4.22 (m, 2H), 3.60 (m, 2H), 3.54 (m, 2H), 3.46 (m, 2H), 3.43 (m, 18H), 3.36 (m, 4H), 3.16 (s, 3H), 3.04 (m, 4H), 2.83 (m, 6H), 2.56 (m, 1H), 2.43 (s, 3H), 2.32 (m, 2H), 2.03 (s, 3H), 1.87 (m, 3H). MS (ESI) m/z 1216.7 (M+H)⁺.

### Example 44

### (7R,16R)-19,23-dichloro-1-(4-fluorophenyl)-20,22-dimethyl-16-[(4-methylpiperazin-1-yl)methyl]-10-({2-[1-(2,5,8,11,14,17,20,23,26,29,32,35,38-tridecaoxanonatriacontan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 44A

### 1-(((tert-butyldimethylsilyl)oxy)methyl)cyclobutanecarbonitrile

1-(Hydroxymethyl)cyclobutanecarbonitrile (2 g) was dissolved in dichloromethane (36 mL) then imidazole (2.45 g) and tert-butyldimethylchlorosilane (3.53 g) were added and the resulting mixture was stirred at room temperature for 4 hours. The mixture was then concentrated onto silica gel and purification by flash chromatography on a CombiFlash® Teledyne Isco system using a Teledyne Isco RediSep® Rf gold 80 g silica gel column (eluting 0-15% ethyl acetate/heptane) afforded the title compound. MS (APCI) m/z 226.5 (M+H)⁺.

### Example 44B

### 1-(((tert-butyldimethylsilyl)oxy)methyl)cyclobutanecarboximidamide

A 2 M solution of trimethylaluminum in toluene (15.37 mL) was slowly added to a magnetically stirred suspension of ammonium chloride (1.645 g) in toluene (38.0 mL) at 0° C. under nitrogen. After the addition, the ice water bath was removed and the mixture was stirred at room temperature for 2 hours until gas evolution (CH₄) had ceased. Example 44A (3.85 g) was added as a toluene (20 mL) solution and the mixture was stirred at 80° C. under nitrogen for 12 hours. The mixture was cooled with an ice water bath and quenched carefully with 100 mL of methanol and stirred at room temperature for 2 hours. The material was removed through filtration and washed with methanol. The combined filtrate was concentrated to afford the crude title compound. MS (APCI) m/z 243.4 (M+H)⁺.

### Example 44C

### 2-(1-(((tert-butyldimethylsilyl)oxy)methyl)cyclobutyl)-4-(dimethoxymethyl)pyrimidine

Example 44B (4.12 g) and 4-(dimethylamino)-1,1-dimethoxybut-3-en-2-one (5.89 g) were taken up in ethanol (24 mL) and to this was added a 21% ethanol solution of sodium ethoxide (33.1 g) which warmed the reaction mildly. The thick mixture was heated at 80° C. for 15 hours then cooled back to ambient temperature. The mixture was concentrated, saturated aqueous sodium bicarbonated was added (150 mL) and the mixture stirred for 2 minutes. The mixture was poured into a 250 mL separatory funnel and extracted with three portions of dichloromethane. The organic layers were combined and the resulting solution was dried over anhydrous magnesium sulfate, filtered and concentrated onto silica gel. Purification by flash chromatography on a CombiFlash® Teledyne Isco system using a Teledyne Isco RediSep® Rf gold 40 g silica gel column (eluting with 5-80% ethyl acetate/heptane) afforded the title compound. MS (APCI) m/z 353.4 (M+H)⁺.

### Example 44D

### (1-(4-(dimethoxymethyl)pyrimidin-2-yl)cyclobutyl)methanol

To a stirring mixture of Example 44C (11.3 g) in 100 mL of tetrahydrofuran was added 96 mL of 1 molar tetrabutyl ammonium fluoride and the mixture was stirred at room temperature for 1 hour. The mixture was concentrated onto silica gel and purification by flash chromatography on a CombiFlash® Teledyne Isco system using a Teledyne Isco RediSep® Rf gold 220 g silica gel column (eluting 30-100% ethyl acetate/heptaneane) afforded the title compound. MS (APCI) m/z 239.4 (M+H)⁺.

### Example 44E

### 2,5,8,11,14,17,20,23,26,29,32,35-dodecaoxaheptatriacontan-37-yl 4-methylbenzenesulfonate

A mixture of 2,5,8,11,14,17,20,23,26,29,32,35-dodecaoxaheptatriacontan-37-ol (500 mg) with triethylamine (0.4 mL) in 10 mL of dichloromethane was stirred at 0° C. and para-toluenesulfonyl chloride (0.255 g) was added in one portion. The cooling bath was removed to allow for the reaction mixture to stir at room temperature for 1 hour. The mixture was concentrated onto silica gel and purification by flash chromatography on a CombiFlash® Teledyne Isco system using a Teledyne Isco RediSep® Rf gold 40 g silica gel column (eluting with 30-100% ethyl acetate/heptaneane) afforded the title compound. MS (APCI) m/z 715.6 (M+H)⁺.

### Example 44F

### 2-(1-(2,5,8,11,14,17,20,23,26,29,32,35,38-tridecaoxanonatriacontyl)cyclobutyl)-4-(dimethoxymethyl)pyrimidine

To a stirring solution of Example 44D (74 mg) and Example 44E (44 mg) in 3.5 mL of acetonitrile was added sodium hydride (81 mg) in one portion and the mixture was stirred at 45° C. overnight. After cooling to ambient temperature, a few drops of saturated aqueous ammonium chloride were added and the mixture was concentrated onto silica gel. Purification by flash chromatography on a CombiFlash® Teledyne Isco system using a Teledyne Isco RediSep® Rf gold 40 g silica gel column (eluting with solvent A=2:1 ethyl acetate:ethanol; solvent B=heptane, 10-100% A to B) afforded the title compound. MS (APCI) m/z 781.4 (M+H)⁺.

### Example 44G

### 2-(1-(2,5,8,11,14,17,20,23,26,29,32,35,38-tridecaoxanonatriacontyl)cyclobutyl)pyrimidine-4-carbaldehyde

Example 44G was synthesized according to the procedure described for Example 29G, substituting Example 44F for Example 29F. MS (APCI) m/z 735.3 (M+H)⁺.

### Example 44H

### (2-(1-(2,5,8,11,14,17,20,23,26,29,32,35,38-tridecaoxanonatriacontyl)cyclobutyl)pyrimidin-4-yl)methanol

Example 44H was synthesized according to the procedure described for Example 29H, substituting Example 44G for Example 29G. MS (APCI) m/z 737.4 (M+H)⁺.

### Example 441

### tert-butyl (7R,16R)-19,23-dichloro-1-(4-fluorophenyl)-20,22-dimethyl-16-[(4-methylpiperazin-1-yl)methyl]-10-({2-[1-(2,5,8,11,14,17,20,23,26,29,32,35,38-tridecaoxanonatriacontan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

Example 44I was synthesized according to the procedure described for Example 291, substituting Example 44H for Example 29H. MS (APCI) m/z 1147.4 (M+H)⁺.

### Example 44J

### (7R,16R)-19,23-dichloro-1-(4-fluorophenyl)-20,22-dimethyl-16-[(4-methylpiperazin-1-yl)methyl]-10-({2-[1-(2,5,8,11,14,17,20,23,26,29,32,35,38-tridecaoxanonatriacontan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 44J was synthesized according to the procedure described for Example 29J, substituting Example 441 for Example 291. ¹H NMR (500 MHz, dimethylsulfoxide-d₆) δ ppm 8.75 (d, 1H), 8.73 (s, 1H), 7.43 (d, 1H), 7.24-7.16 (m, 2H), 7.16-7.08 (m, 2H), 6.87 (d, 1H), 6.73 (dd1H), 6.25-6.17 (m, 1H), 5.88-5.77 (m, 1H), 5.19-5.03 (m, 2H), 4.95-4.84 (m, 1H), 4.50-4.39 (m, 2H), 3.86 (s, 2H), 3.60 (dd, 1H), 3.54-3.40 (m, 48H), 3.23 (s, 3H), 3.00-2.91 (m, 1H), 2.75-2.61 (m, 2H), 2.49-2.28 (m, 10H), 2.23-2.11 (m, 5H), 2.04-1.92 (m, 7H), 1.86-1.73 (m, 1H).

### Example 45

### (7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 45A

### 2-(1-(2,5,8,11,14,17,20,23,26,29,32,35,38-tridecaoxanonatriacontyl)cyclobutyl)pyrimidine-4-carbaldehyde

Example 45A was synthesized according to the procedure described for Example 44F, substituting 2-(2-(2-methoxyethoxy)ethoxy)ethyl 4-methylbenzenesulfonate for Example 44E. MS (APCI) m/z 385.4 (M+H)⁺.

### Example 45B

### 2-(1-(2,5,8,11-tetraoxadodecyl)cyclobutyl)pyrimidine-4-carbaldehyde

Example 45B was synthesized according to the procedure described for Example 29G, substituting Example 45A for Example 29F. MS (APCI) m/z 339.4 (M+H)⁺.

### Example 45C

### (2-(1-(2,5,8,11-tetraoxadodecyl)cyclobutyl)pyrimidin-4-yl)methanol

Example 45C was synthesized according to the procedure described for Example 29H, substituting Example 45B for Example 29G. MS (APCI) m/z 341.3 (M+H)⁺.

### Example 45D

### tert-butyl (7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

Example 45D was synthesized according to the procedure described for Example 291, substituting Example 45C for Example 29H. MS (APCI) m/z 1131.7 (M+H)⁺.

### Example 45E

### (7R,16R)-19,23-dichloro-1-(4-fluorophenyl)-20,22-dimethyl-16-[(4-methylpiperazin-1-yl)methyl]-10-({2-[1-(2,5,8,11-tetraoxadodecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 45E was synthesized according to the procedure described for Example 29J, substituting Example 45D for Example 291. ¹H NMR (500 MHz, dimethylsulfoxide-d₆) δ ppm 8.74 (d, 1H), 8.71 (s, 1H), 7.43 (d, 1H), 7.22-7.16 (m, 2H), 7.16-7.09 (m, 2H), 6.85 (d, 1H), 6.71 (dd, 1H), 6.21 (dd, J=5.6, 3.3 Hz, 1H), 5.90-5.82 (m, 1H), 5.18-5.02 (m, 2H), 4.94-4.86 (m, 1H), 4.51-4.37 (m, 2H), 3.86 (s, 2H), 3.58 (dd, 1H), 3.49-3.34 (m, 12H), 3.20 (s, 3H), 3.00-2.91 (m, 1H), 2.74-2.60 (m, 2H), 2.49-2.34 (m, 10H), 2.20 (s, 3H), 2.18-2.10 (m, 2H), 2.05-1.91 (m, 7H), 1.86-1.72 (m, 1H). MS (APCI) m/z 1076.0 (M+H)⁺.

### Example 46

### (7R,16R)-19,23-dichloro-1-(4-fluorophenyl)-10-{[2-(6-{2-[2-(2-methoxyethoxy)ethoxy]ethoxy}pyridin-3-yl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 46A

### 5-bromo-2-(2-(2-(2-methoxyethoxy)ethoxy)ethoxy)pyridine

To a solution of 5-bromo-2-chloropyridine (5 g) and 2-(2-(2-methoxyethoxy)ethoxy)ethanol (6.40 g) in dimethylsulfoxide (50 mL) was added sodium hydride (0.624 g) at 20° C. under nitrogen in flow. The reaction mixture was stirred at 60° C. for 10 hours under nitrogen atmosphere, diluted with water (20 mL) at 25° C. and extracted with ethyl acetate (3×30 mL). The combined organic layers were dried over sodium sulfate, filtered and concentrated to provide the title compound. MS (ESI) m/z 319.9 (M+H)⁺.

### Example 46B

### (6-(2-(2-(2-methoxyethoxy)ethoxy)ethoxy)pyridin-3-yl)boronic Acid

To a solution of Example 46A (3.3 g) in 1,4-dioxane (150 mL) was added 4,4,4′,4′,5,5,5′,5′-octamethyl-2,2′-bi(1,3,2-dioxaborolane) (3.93 g), potassium acetate (2.023 g) and PdCl₂(dppf)-dichloromethane adduct (1.683 g) at 20° C. The mixture was stirred at 100° C. for 12 hours under nitrogen atmosphere, cooled to 25° C. and filtered. The filtrate was concentrated to give the title compound which was directly used for the next step without further purification. MS (ESI) m/z 286 (M+H)⁺.

### Example 46C

### 2-(6-(2-(2-(2-methoxyethoxy)ethoxy)ethoxy)pyridin-3-yl)pyrimidine-4-carboxylic Acid

To a solution of Example 46B (2.94 g) in 1,4-dioxane (200 mL) were added 2-chloropyrimidine-4-carboxylic acid (1.5 g), sodium bicarbonate (1.590 g) and Pd(PPh₃)₄ (1.093 g) at 25° C. under nitrogen flow. The reaction mixture was stirred at 110° C. for 16 hours under nitrogen atmosphere, cooled down to 20° C. and filtered. The filtrate was dissolved into 10 ml of water and the water phase was extracted with ethyl acetate (50 mL) three times. The water phase was purified by reverse phase HPLC to provide the title compound. MS (ESI) m/z 364.2 (M+H)⁺.

### Example 46D

### methyl 2-(6-(2-(2-(2-methoxyethoxy)ethoxy)ethoxy)pyridin-3-yl)pyrimidine-4-carboxylate

To a solution of Example 46C (3 g) in methanol (40 mL) was added sulfuric acid (81 mg) at 0° C. The reaction mixture was heated at 80° C. for 18 hours, poured into water (80 mL) and extracted with ethyl acetate (3×80 mL). The combined organic layers were washed with brine (3×50 mL), dried over sodium sulfate, filtered, and concentrated. The residue was purified by column chromatography on silica gel (eluting with petroleum ether:ethyl acetate=10:1 to 1:1) to provide the title compound. ¹H NMR (400 MHz, CDCl₃) δ ppm 9.27 (d, 1H), 8.98 (d, 1H), 8.65 (dd, 1H), 7.82 (d, 1H), 6.89 (d, 1H), 4.61-4.55 (m, 2H), 4.04 (s, 3H), 3.93-3.86 (m, 2H), 3.76-3.73 (m, 2H), 3.71-3.64 (m, 4H), 3.59-3.53 (m, 2H), 3.38 (s, 3H).

### Example 46E

### (2-(6-(2-(2-(2-methoxyethoxy)ethoxy)ethoxy)pyridin-3-yl)pyrimidin-4-yl)methanol

To a solution of Example 46D (2.4 g) in methanol (40 mL) was added sodium borohydride (0.43 g) at 0° C. The reaction was stirred at 20° C. for 1 hour, poured into water (100 mL) and extracted with ethyl acetate (3×100 mL). The combined organic layers were washed with brine (3×50 mL) and dried over sodium sulfate. After filtration, the filtrate was concentrated to give a residue which was triturated with ethyl acetate (5 mL) and petroleum ether (20 mL). The material was collected by suction filtration to provide the title compound. ¹H NMR (400 MHz, CDCl₃) δ ppm 9.21 (d, 1H), 8.71 (d, 1H), 8.58 (dd. H), 7.17 (d, 1H), 6.87 (d, 1H), 4.79 (s, 2H), 4.60-4.54 (m, 2H), 3.91-3.85 (m, 2H), 3.77-3.73 (m, 2H), 3.71-3.68 (m, 2H), 3.66 (dd, 2H), 3.58-3.52 (m, 2H), 3.38 (s, 3H). MS (ESI) m/z 350 (M+H)⁺.

### Example 46F

### tert-butyl (7R,16R)-19,23-dichloro-1-(4-fluorophenyl)-10-{[2-(6-{2-[2-(2-methoxyethoxy)ethoxy]ethoxy}pyridin-3-yl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 28E by replacing Example 12P and Example 28D with Example 16N and Example 46E, respectively. MS (APCI) m/z 1142.4 (M+H)⁺.

### Example 46G

### (7R,16R)-19,23-dichloro-1-(4-fluorophenyl)-10-{[2-(6-{2-[2-(2-methoxyethoxy)ethoxy]ethoxy}pyridin-3-yl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 28F by replacing Example 28E with Example 46F. ¹H NMR (501 MHz, dimethylsulfoxide-d₆) δ ppm 9.12 (d, 1H), 8.86 (d, 1H), 8.73 (s, 1H), 8.58 (dd, 1H), 7.52 (d, 1H), 7.19 (t, 2H), 7.17-7.10 (m, 2H), 6.97 (d, 1H), 6.88 (d, 1H), 6.74 (dd, 1H), 6.22 (dd, 1H), 5.82 (d, 1H), 5.25 (d, 1H), 5.18 (d, 1H), 4.86 (p, 1H), 4.49-4.42 (m, 4H), 3.80-3.74 (m, 2H), 3.55 (s, 2H), 3.68-3.48 (m, 8H), 3.01-2.93 (m, 1H), 2.70-2.62 (m, 2H), 2.18 (s, 3H), 1.97 (d, 6H). MS (ESI) m/z 1084.3 (M+H)⁺.

### Example 47

### (7R,16R)-19,23-dichloro-1-(4-fluorophenyl)-20,22-dimethyl-16-[(4-methylpiperazin-1-yl)methyl]-10-[(2-{4-[(2,5,8,11,14,17,20,23,26,29,32-undecaoxatetratriacontan-34-yl) carbamoyl]phenyl}pyrimidin-4-yl)methoxy]-7,8,15,6-tetrahydro-18,21-etheno-13,9-(metheno)-6,14,17-trioxa-2-thia-3,5-diazacyclononadeca[1,2,3-cd]indene-7-carboxylic Acid

### Example 47A

### 1-(4-(4,4,5,5-tetramethyl-1,3,2-dioxaborolan-2-yl)phenyl)-5,8,11,14,17,20,23,26,29,32,35-undecaoxa-2l2-azahexatriacontan-1-one

4-(4,4,5,5-Tetramethyl-1,3,2-dioxaborolan-2-yl)benzoic acid (100 mg) and 2,5,8,11,14,17,20,23,26,29,32-undecaoxatetratriacontan-34-amine (229 mg) were dissolved in dichloromethane (2 mL). N¹-((Ethylimino)methylene)-N³,N³-dimethylpropane-1,3-diamine hydrochloride (162 mg) and N,N-dimethylpyridin-4-amine (73.9 mg) were added. The solution was mixed at room temperature overnight. The solution was concentrated under vacuum and purified by flash column chromatography using a gradient of 0-20% methanol in dichloromethane. The solvent was removed under vacuum to yield the title compound. ¹H NMR (500 MHz, dimethylsulfoxide-d₆) δ ppm 8.56 (t, 1H), 7.85 (d, 2H), 7.74 (d, 2H), 3.56-3.46 (m, 44H), 3.24 (s, 3H), 1.31 (s, 12H). MS (ESI) m/z 763.0 (M+NH₄)⁺.

### Example 47B

### 1-(4-(4-(hydroxymethyl)pyrimidin-2-yl)phenyl)-5,8,11,14,17,20,23,26,29,32,35-undecaoxa-2l2-azahexatriacontan-1-one

The title compound was prepared by substituting Example 47A for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.92 (d, 1H), 8.63 (t, 1H), 8.45 (d, 2H), 7.99 (d, 2H), 7.54 (d, 1H), 5.71 (t, 1H), 4.67 (d, 2H), 3.53-3.47 (m, 44H), 3.23 (s, 3H). MS (ESI) m/z 726.2 (M−H)⁻.

### Example 47C

### (7R,16R)-19,23-dichloro-1-(4-fluorophenyl)-20,22-dimethyl-16-[(4-methylpiperazin-1-yl)methyl]-10-[(2-{4-[(2,5,8,11,14,17,20,23,26,29,32-undecaoxatetratriacontan-34-yl) carbamoyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 47B for Example 38D in Example 38E. ¹H NMR (500 MHz, dimethylsulfoxide-d₆) δ ppm 8.87 (d, 1H), 8.68 (s, 1H), 8.58 (t, 1H), 8.40 (d, 2H), 7.93 (d, 2H), 7.51 (d, 1H), 7.15-7.06 (m, 4H), 6.85 (d, 1H), 6.72 (d, 1H), 6.21 (m, 1H), 5.73 (s, 1H), 5.19 (q, 2H), 4.81 (m, 1H), 4.38 (m, 2H), 3.61 (m, 2H), 3.51-3.42 (m, 48H), 3.16 (s, 3H), 2.94 (d, 2H), 2.68-2.52 (m, 4H), 2.29 (s, 3H), 1.93 (s, 3H), 1.88 (s, 3H). MS (ESI) m/z 1464.7 (M+H)⁺.

### Example 48

### (7R,16R)-19,23-dichloro-10-{[2-(6,6-difluoro-2-azaspiro[3.3]heptan-2-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 48A

### (2-(6,6-difluoro-2-azaspiro[3.3]heptan-2-yl)pyrimidin-4-yl)methanol

A mixture of (2-chloropyrimidin-4-yl)methanol (220 mg), 6,6-difluoro-2-azaspiro[3.3]heptane hydrochloride (297 mg) and triethylamine (616 mg) in dioxane (4 mL) was heated in a Q-tube for 7 hours at 80° C. The stirring was then continued at room temperature overnight. Excess water was added, followed by extraction with ethyl acetate, washing of the combined organic layers with water and drying (MgSO₄). The crude product was purified by chromatography on silica gel using a Grace Reveleris system (12 g Grace Reveleris column, eluting with 1-50% dichloromethane/ethyl acetate) providing the title compound. MS (APCI) m/z 242.2 (M+H)⁺.

### Example 48B

### (2-(6,6-difluoro-2-azaspiro[3.3]heptan-2-yl)pyrimidin-4-yl)methyl methanesulfonate

Triethylamine (68.5 mg) was added to an ice-cooled solution of Example 48A (81.7 mg) in dichloromethane (5 mL). After addition of methanesulfonyl chloride (46.6 mg) the stirring was continued for 3 hours under ice-cooling. The reaction mixture was diluted with dichloromethane, washed with water, dried (MgSO₄), filtered, and the solvent was removed in vacuo. The crude title compound obtained was used without further purification.

### Example 48C

### tert-butyl (7R,16R)-19,23-dichloro-10-{[2-(6,6-difluoro-2-azaspiro[3.3]heptan-2-yl)pyrimidin-4-yl]methoxy}-1-(4-fluorophenyl)-20,22-dimethyl-16-[(4-methylpiperazin-1-yl)methyl]-7,8,15,6-tetrahydro-18,21-etheno-9,13-(metheno)-6,14,17-trioxa-2-thia-3,5-diazacyclononadeca[1,2,3-cd]indene-7-carboxylate

Cesium carbonate (48.3 mg) was added to a mixture of Example 16N (40 mg) and Example 48B (31.5 mg) in dimethylformamide (0.4 mL). After stirring overnight at room temperature, a 1:1 mixture of water and saturated aqueous NaHCO₃ solution (3 mL) was added. The suspension obtained was stirred for 2 minutes, and the precipitate formed was filtered and washed with water. The crude product was purified by chromatography on silica gel using a Grace Reveleris system (12 g Grace Reveleris column, eluting with 1-10% dichloromethane/methanol) providing the title compound. MS (APCI) m/z 1032.4 (M+H)⁺.

### Example 48D

### (7R,16R)-19,23-dichloro-10-{[2-(6,6-difluoro-2-azaspiro[3.3]heptan-2-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Trifluoroacetic acid (188 mg) was added to a solution of Example 48C (34 mg) in dichloromethane (0.4 mL) and the reaction mixture was stirred overnight at room temperature. Removal of the solvent, followed by purification by HPLC (Waters XBridge C8 19×150 mm 5 μm column, gradient 5-100% acetonitrile+0.2% ammonium hydroxide in water+0.2% ammonium hydroxide) provided the title compound. ¹H NMR (600 MHz, dimethylsulfoxide-d₆) δ ppm 8.69 (s, 1H), 8.32 (d, 1H), 7.21-7.17 (m, 2H), 7.14-7.10 (m, 2H), 6.82-6.77 (m, 2H), 6.70 (m, 1H), 6.11 (s, 1H), 5.86 (s, 1H), 4.97-4.87 (m, 3H), 4.46-4.39 (m, 2H), 4.13 (s, 4H), 3.50 (m, 1H), 2.92-2.84 (m, 5H), 2.71-2.64 (m, 2H), 2.48-2.28 (m, 8H), 2.17 (s, 3H), 2.00-1.92 (m, 6H). MS (ESI) m/z 976.4 (M+H)⁺.

### Example 49

### (7R,16R)-10-({2-[4-(carboxymethyl)piperidin-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 49A

### methyl 2-(1-(4-(hydroxymethyl)pyrimidin-2-yl)piperidin-4-yl)acetate

A solution of methyl 2-(piperidin-4-yl)acetate, hydrochloric acid salt (320 mg), (2-chloropyrimidin-4-yl)methanol (200 mg) and N,N-diisopropylethylamine (770 μL) in acetonitrile (3.5 mL) was heated to 80° C. for 2 hours and stirred at room temperature overnight. The reaction was diluted with water and extracted with ethyl acetate three times. The combined organic layers were washed with brine, dried over anhydrous sodium sulfate, filtered and concentrated. The residue was purified by normal phase MPLC on a Teledyne Isco CombiFlash® Rf+24 g gold silica gel column eluting with 0-60% ethyl acetate in dichlormethane. Desired fractions were concentrated, and the residue was purified by RP-HPLC on a Gilson PLC 2020 using a Luna™ column (250×50 mm, 10 mm) (5-65% over 30 minutes with acetonitrile in water containing 0.01% trifluoroacetic acid). Desired fractions were combined, washed with saturated sodium bicarbonate and extracted with dichloromethane three times. The organic layers were dried over anhydrous sodium sulfate, filtered and concentrated to give the title compound. ¹H NMR (400 MHz, dimethylsulfoxide-d₆) δ ppm 8.29 (d, 1H), 6.66 (s, 1H), 5.41-5.29 (m, 1H), 4.71-4.55 (m, 2H), 4.33 (d, 2H), 3.59 (s, 3H), 2.91-2.74 (m, 2H), 2.26 (d, 2H), 2.04-1.86 (m, 1H), 1.77-1.61 (m, 2H), 1.21-1.00 (m, 2H).

### Example 49B

### tert-butyl (7R,16R)-19,23-dichloro-1-(4-fluorophenyl)-10-({2-[4-(2-methoxy-2-oxoethyl)piperidin-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (25 mg) and Example 49A (12.29 mg, 0.046 mmol) in toluene (80 μL) and tetrahydrofuran (100 μL) was added triphenylphosphine (24 mg) followed by N,N,N′,N′-tetramethylazodicarboxamide (16 mg), and the reaction was allowed to stir at 50° C. for 5 hours. The reaction was cooled, diluted with ethyl acetate, filtered over diatomaceous earth and concentrated. The residue was purified by normal phase MPLC on a Teledyne Isco CombiFlash® Rf+4 g gold silica gel column eluting with 0-5.5% methanol in dichlormethane to give the title compound.

### Example 49C

### (7R,16R)-19,23-dichloro-1-(4-fluorophenyl)-10-({2-[4-(2-methoxy-2-oxoethyl)piperidin-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 49B (21 mg) in dichloromethane (100 μL) was added trifluoroacetic acid (100 μL), and the reaction was allowed to stir for 5 hours. The reaction was concentrated under a stream of nitrogen and taken up in water and acetonitrile. The mixture was purified by RP-HPLC on a Gilson PLC 2020 using a Luna™ column (250×50 mm, 10 mm) (5-85% over 30 minutes with acetonitrile in water containing 0.01% trifluoroacetic acid) to give the title compound. ¹H NMR (400 MHz, dimethylsulfoxide-d₆) δ ppm 8.75 (s, 1H), 8.30 (d, 1H), 7.27-7.08 (m, 5H), 6.87-6.75 (m, 2H), 6.66 (d, 1H), 6.23 (dd, 1H), 5.81-5.73 (m, 1H), 5.02-4.83 (m, 3H), 4.69-4.57 (m, 2H), 4.52-4.37 (m, 2H), 3.59 (s, 3H), 3.16-2.75 (m, 12H), 2.27 (d, 2H), 2.03-1.89 (m, 6H), 1.75-1.63 (m, 2H), 1.18-1.01 (m, 2H).

### Example 49D

### (7R,16R)-10-({2-[4-(carboxymethyl)piperidin-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 49C (17 mg) in tetrahydrofuran (200 μL) and methanol (200 μL) at room temperature was added a solution of lithium hydroxide (8.3 mg) in water (200 μL), and the reaction was stirred for 3 hours. The reaction was quenched with trifluoroacetic acid (35 μL), taken up in dimethylsulfoxide and purified by RP-HPLC on a Gilson PLC 2020 using a Luna™ column (250×50 mm, 10 mm) (5-75% over 30 minutes with acetonitrile in water containing 10 mM ammonium acetate) to give the title compound. ¹H NMR (400 MHz, dimethylsulfoxide-d₆) δ ppm 8.71 (s, 1H), 8.29 (d, 1H), 7.24-7.08 (m, 5H), 6.79 (d, 1H), 6.74-6.64 (m, 2H), 6.22-6.14 (m, 1H), 5.87-5.78 (m, 1H), 4.99-4.83 (m, 3H), 4.68-4.57 (m, 2H), 4.49-4.36 (m, 2H), 2.97-2.78 (m, 4H), 2.74-2.58 (m, 4H), 2.43 (br s, 4H), 2.21 (s, 3H), 2.15 (d, 2H), 2.01-1.88 (m, 7H), 1.76-1.64 (m, 2H), 1.15-1.00 (m, 1H).

### Example 50

### (7R,16R)-19,23-dichloro-1-(4-fluorophenyl)-20,22-dimethyl-16-[(4-methylpiperazin-1-yl)methyl]-10-({2-[4-(35-oxo-2,5,8,11,14,17,20,23,26,29,32-undecaoxa-36-azaheptatriacontan-37-yl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 50A

### 37-(4-(4,4,5,5-tetramethyl-1,3,2-dioxaborolan-2-yl)phenyl)-2,5,8,11,14,17,20,23,26,29,32-undecaoxa-36l2-azaheptatriacontan-35-one

The title compound was prepared by substituting 2,5,8,11,14,17,20,23,26,29,32-undecaoxapentatriacontan-35-oic acid for 4-(4,4,5,5-tetramethyl-1,3,2-dioxaborolan-2-yl)benzoic acid and (4-(4,4,5,5-tetramethyl-1,3,2-dioxaborolan-2-yl)phenyl) methanamine for 2,5,8,11,14,17,20,23,26,29,32-undecaoxatetratriacontan-34-amine in Example 47A. ¹H NMR (500 MHz, dimethylsulfoxide-d₆) δ ppm 8.36 (t, 1H), 7.61 (d, 2H), 7.26 (d, 2H), 4.29 (d, 2H), 3.63 (t, 2H), 3.50 (m, 38H), 3.43 (m, 2H), 3.24 (s, 3H), 2.38 (t, 2H), 1.28 (s, 12H). MS (ESI) m/z 777.3 (M+NH₄)⁺.

### Example 50B

### 37-(4-(4-(hydroxymethyl)pyrimidin-2-yl)phenyl)-2,5,8,11,14,17,20,23,26,29,32-undecaoxa-36l2-azaheptatriacontan-35-one

The title compound was prepared by substituting Example 50A for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.86 (d, 1H), 8.43 (t, 1H), 8.31 (d, 2H), 7.48 (d, 1H), 7.39 (d, 2H), 5.68 (t, 1H), 4.63 (d, 2H), 4.36 (d, 2H), 3.66 (t, 2H), 3.52 (m, 38H), 3.45-3.41 (m, 2H), 3.23 (s, 3H), 2.42 (t, 2H). MS (ESI) m/z 742.5 (M+H)⁺.

### Example 50C

### (7R,16R)-19,23-dichloro-1-(4-fluorophenyl)-20,22-dimethyl-16-[(4-methylpiperazin-1-yl)methyl]-10-({2-[4-(35-oxo-2,5,8,11,14,17,20,23,26,29,32-undecaoxa-36-azaheptatriacontan-37-yl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 50B for Example 38D in Example 38E. ¹H NMR (500 MHz, dimethylsulfoxide-d₆) δ ppm 8.87 (d, 1H), 8.71 (s, 1H), 8.43 (t, 1H), 8.36 (d, 2H), 7.55 (m, 1H), 7.40 (d, 2H), 7.19 (t, 2H), 7.15-7.10 (m, 2H), 6.87 (m, 1H), 6.73 (m, 1H), 6.55 (s, 1H), 5.88 (s, 1H), 5.22 (q, 2H), 4.90 (m, 1H), 4.44 (d, 2H), 4.36 (d, 1H), 3.66 (t, 2H), 3.49 (m, 46H), 3.42 (m, 2H), 3.23 (s, 3H), 2.97 (m, 2H), 2.67 (m, 3H), 2.41 (t, 2H), 2.33 (s, 3H), 1.99 (s, 3H), 1.95 (s, 3H). MS (ESI) m/z 1476.6 (M+H)⁺.

### Example 51

### (7R,16R)-19,23-dichloro-10-[(2-{3-[(dimethylphosphoryl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 51A

### (3-(4-(((tert-butyldimethylsilyl)oxy)methyl)pyrimidin-2-yl)phenyl)methanol

A mixture of Example 38A (700 mg), 3-(hydroxymethyl)phenylboronic acid (411 mg), and tetrakis(triphenylphosphine)palladium (0) (156 mg) in tetrahydrofuran (9 mL) and saturated aqueous sodium bicarbonate solution (5.14 mL) was evacuated and backfilled with nitrogen twice. The mixture was stirred at 70° C. overnight. The mixture was diluted with water and extracted with three portions of ethyl acetate. The combined organic layers were dried over anhydrous magnesium sulfate, filtered and concentrated. The residue was purified by silica gel flash chromatography on AnaLogix IntelliFlash²⁸⁰ system eluting with 0-35% ethyl acetate in hexanes to give the title compound. MS (ESI) m/z 331.2 (M+H)⁺.

### Example 51B

### 2-(3-(bromomethyl)phenyl)-4-(((tert-butyldimethylsilyl)oxy)methyl)pyrimidine

To a stirring solution of Example 51A (285 mg) and triphenylphosphine (339 mg) in dichloromethane (6 mL) was added carbon tetrabromide (429 mg). The mixture was stirred for 3 hours. The reaction mixture was purified by silica gel flash chromatography on AnaLogix IntelliFlash²⁸⁰ system eluting with 0-20% ethyl acetate in hexanes to give the title compound. MS (ESI) m/z 395.2 (M+H)⁺.

### Example 51C

### (3-(4-(((tert-butyldimethylsilyl)oxy)methyl)pyrimidin-2-yl)benzyl)dimethylphosphine oxide

Sodium bis(trimethylsilyl)amide (0.638 mL) was added dropwise to a solution of dimethylphosphine oxide (49.8 mg) in tetrahydrofuran (2.5 mL) and the mixture was stirred at ambient temperature for 15 minutes. The turbid solution was added dropwise to a solution of Example 51B (251 mg) in tetrahydrofuran (2.5 mL). The mixture was stirred at ambient for 3 hours. The reaction mixture was diluted with water and extracted three times with ethyl acetate. The organic layer was washed with brine, dried over sodium sulfate, filtered, and concentrated. The crude product was purified by silica gel flash chromatography on AnaLogix IntelliFlash²⁸⁰ system eluting with 5-20% methanol in dichloromethane to give the title compound. MS (ESI) m/z 391.4 (M+H)⁺.

### Example 51D

### (3-(4-(hydroxymethyl)pyrimidin-2-yl)benzyl)dimethylphosphine oxide

To a solution of Example 51C (146 mg) in methanol (3 mL) was added cesium fluoride (114 mg). The mixture was stirred for 1 hour, concentrated and the residue was purified by silica gel flash chromatography on AnaLogix IntelliFlash²⁸⁰ system eluting with 5-20% methanol in dichloromethane to give the title compound. MS (ESI) m/z 277.2 (M+H)⁺.

### Example 51E

### tert-butyl (7R,16R)-19,23-dichloro-10-[(2-{3-[(dimethylphosphoryl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 51D (64 mg and triethylamine (70.3 mg) in dichloromethane (2.5 mL) at 0° C. was added methanesulfonyl chloride (39.8 mg). The mixture was stirred for 40 minutes. The mixture was purified by silica gel flash chromatography, eluting with 2-10% methanol in dichloromethane to give the mesylate. To this, along with Example 16N (60 mg) in dimethylformamide (0.4 mL), was added cesium carbonate (72.4 mg) and the reaction mixture was stirred for 90 minutes. The mixture was diluted with water and extracted three times with dichloromethane. The organic layer was washed with brine, dried over sodium sulfate, filtered, and concentrated. The crude product was 20 purified by silica gel flash chromatography on AnaLogix IntelliFlash²⁸⁰ system eluting with 5-16% methanol in dichloromethane to give the title compound. MS (ESI) m/z 1069.1 (M+H)⁺.

### Example 51F

### (7R,16R)-19,23-dichloro-10-[(2-{3-[(dimethylphosphoryl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 51E (60 mg) in dichloromethane (0.40 mL) was added trifluoroacetic acid (0.40 mL). The mixture was stirred at ambient temperature for 3 hours and concentrated. The residue was dissolved in N,N-dimethylformamide and acetonitrile and purified by reverse phase chromatography using a 5-65% gradient of acetonitrile in water (with 0.1% ammonium acetate) over 30 minutes on a Grace Reveleris equipped with a Luna™ column: C18(2), 100 Å, 250×50 mm. The fractions containing the desired compound were combined, frozen and lyophilized to isolate the title compound. ¹H NMR (501 MHz, dimethylsulfoxide-d₆) δ ppm 8.88 (d, 1H), 8.73 (s, 1H), 8.42-8.23 (m, 2H), 7.53 (d, 1H), 7.50-7.34 (m, 2H), 7.26-7.08 (m, 4H), 6.88 (d, 1H), 6.74 (dd, 1H), 6.23 (dd, 1H), 5.82 (d, 1H), 5.31-5.06 (m, 2H), 4.86 (m, 1H), 4.44 (d, 2H), 3.73-2.27 (m, 14H), 2.18 (s, 3H), 1.98 (s, 3H), 1.95 (s, 3H), 1.37 (s, 3H), 1.35 (s, 3H). MS (ESI) m/z 1011.4 (M+H)⁺.

### Example 52

### (7R,16R)-19,23-dichloro-1-(4-fluorophenyl)-10-{[2-(4-{2-[2-(2-methoxyethoxy)ethoxy]ethyl}piperidin-1-yl)pyrimidin-4-yl]methoxy}-20,22-dimethyl-16-[(4-methylpiperazin-1-yl)methyl]-7,8,15,6-tetrahydro-18,21-etheno-9,13-(metheno)-6,14,17-trioxa-2-thia-3,5-diazacyclononadeca[1,2,3-cd]indene-7-carboxylic Acid

### Example 52A

### (2-(4-(2-(2-(2-methoxyethoxy)ethoxy)ethyl)piperidin-1-yl)pyrimidin-4-yl)methanol

A mixture of (2-chloropyrimidin-4-yl)methanol (220 mg), 4-(2-(2-(2-methoxyethoxy)ethoxy)ethyl)piperidine (387 mg) and triethylamine (616 mg) in dioxane (4 mL) was heated in a Q-tube for 7 hours at 80° C. The stirring was then continued at room temperature overnight. Excess water was added, followed by extraction with ethyl acetate, washing of the combined organic layers with water, drying over magnesium sulfate, filtration and concentration. The crude title compound obtained was used without further purification.

### Example 52B

### (2-(4-(2-(2-(2-methoxyethoxy)ethoxy)ethyl)piperidin-1-yl)pyrimidin-4-yl)methyl methanesulfonate

Triethylamine (95 mg) was added to an ice-cooled solution of Example 52A (159 mg) in dichlormethane (5 mL). After addition of methanesulfonyl chloride (64 mg) the stirring was continued for 3 hours under ice-cooling. The reaction mixture was diluted with dichlormethane, washed with water, dried over magnesium sulfate, filtered and concentrated. The crude title compound obtained was used without further purification.

### Example 52C

### tert-butyl (7R,16R)-19,23-dichloro-1-(4-fluorophenyl)-10-{[2-(4-{2-[2-(2-methoxyethoxy)ethoxy]ethyl}piperidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Cesium carbonate (60.4 mg) was added to a mixture of Example 16N (50 mg) and Example 52B (51.6 mg) in dimethylformamide (0.2 mL). After stirring for 3 days at room temperature, a 1:1 mixture of water and saturated aqueous NaHCO₃ solution (3 mL) was added. The suspension obtained was stirred for 20 minutes, and the precipitate formed was filtered and washed with water. The crude product was purified by chromatography on silica gel using a CombiFlash® system (4 g RediSep® Gold column, eluting with 1-10% dichloromethane/methanol) providing the title compound. MS (APCI) m/1030.4 (M+H)⁺.

### Example 52D

### (7R,16R)-19,23-dichloro-1-(4-fluorophenyl)-10-{[2-(4-{2-[2-(2-methoxyethoxy)ethoxy]ethyl}piperidin-1-yl)pyrimidin-4-yl]methoxy}-20,22-dimethyl-16-[(4-methylpiperazin-1-yl)methyl]-7,8,15,6-tetrahydro-18,21-etheno-9,13-(metheno)-6,14,17-trioxa-2-thia-3,5-diazacyclononadeca[1,2,3-cd]indene-7-carboxylic Acid

Trifluoroacetic acid (0.17 mL) was added to Example 52C (25 mg) in dichloromethane (0.5 mL). The reaction mixture was stirred overnight at room temperature. Removal of the solvent, followed by purification by HPLC (Waters XBridge C8 19×150 mm 5 μm column, gradient 5-100% acetonitrile+0.2% ammonium hydroxide in water+0.2% ammonium hydroxide) provided the title compound. ¹H NMR (600 MHz, dimethylsulfoxide-d₆) δ ppm 8.71 (s, 1H), 8.29 (d, 1H), 7.22-7.17 (m, 2H), 7.13 (m, 2H), 6.78 (m, 1H), 6.73-6.65 (m, 2H), 6.15 (s, 1H), 5.83 (s, 1H), 4.97-4.86 (m, 3H), 4.67-4.61 (m, 2H), 4.46-4.40 (m, 2H), 3.58-3.40 (m, 11H), 3.24 (s, 3H), 2.93-2.90 (m, 1H), 2.82 (td, 2H), 2.71-2.63 (m, 2H), 2.47-2.26 (m, 8H), 2.17 (s, 3H), 2.01-1.91 (m, 6H), 1.70 (m, 2H), 1.64 (m, 1H), 1.44 (q, 2H), 1.05 (m, 2H). MS (APCI) m/z 1074.4 (M+H)⁺.

### Example 53

### (7R,16R)-19,23-dichloro-1-(4-fluorophenyl)-20,22-dimethyl-16-[(4-methylpiperazin-1-yl)methyl]-10-({2-[4-(2,5,8,11-tetraoxatetradecan-14-yl) piperazin-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 53A

### tert-butyl 4-(4-(hydroxymethyl)pyrimidin-2-yl)piperazine-1-carboxylate

A solution of tert-butyl piperazine-1-carboxylate (620 mg), (2-chloropyrimidin-4-yl)methanol (400 mg) and N,N-diisopropylethylamine (1.5 mL) in acetonitrile (6.9 mL) was heated to 80° C. for 4 hours. The reaction was cooled, diluted with water and extracted with ethyl acetate three times. The combined organic layers were dried over anhydrous sodium sulfate, filtered and concentrated. The residue was purified by normal phase MPLC on a Teledyne Isco CombiFlash® Rf+40 g gold silica gel column eluting with 0-60% ethyl acetate in dichlormethane to give the title compound. ¹H NMR (500 MHz, dimethylsulfoxide-d₆) δ ppm 8.34 (d, 1H), 6.74 (d, 1H), 5.47-5.37 (m, 1H), 4.35 (d, 2H), 3.76-3.61 (m, 4H), 3.43-3.30 (m, 4H), 1.41 (s, 9H).

### Example 53B

### tert-butyl (7R,16R)-10-({2-[4-(tert-butoxycarbonyl) piperazin-1-yl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (50 mg) and Example 53A (27 mg) in toluene (150 μL) and tetrahydrofuran (150 μL) was added triphenylphosphine (49 mg) followed by N,N,N′,N′-tetramethylazodicarboxamide (32 mg), and the reaction was allowed to stir at 50° C. for 3 hours. The reaction was cooled, diluted with ethyl acetate, filtered over diatomaceous earth and the filtrate was concentrated. The residue was purified by normal phase MPLC on a Teledyne Isco CombiFlash® Rf+4 g gold silica gel column eluting with 0-7.5% methanol in dichlormethane to give the title compound. ¹H NMR (500 MHz, dimethylsulfoxide-d₆) δ ppm 8.74 (s, 1H), 8.38 (d, 1H), 7.26-7.13 (m, 5H), 6.86 (d, 1H), 6.82 (dd, 1H), 6.76 (d, 1H), 6.02 (dd, 1H), 5.67 (d, 1H), 5.02-4.85 (m, 2H), 4.80-4.69 (m, 1H), 4.53-4.33 (m, 2H), 3.78-3.67 (m, 2H), 3.65-3.58 (m, 1H), 3.43-3.36 (m, 4H), 2.91-2.82 (m, 1H), 2.71-2.59 (m, 2H), 2.44-2.20 (m, 4H), 2.14 (s, 3H), 2.09 (s, 3H), 1.90 (s, 3H), 1.42 (s, 9H), 1.07 (s, 9H).

### Example 53C

### tert-butyl (7R,16R)-19,23-dichloro-1-(4-fluorophenyl)-20,22-dimethyl-16-[(4-methylpiperazin-1-yl)methyl]-10-{[2-(piperazin-1-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

An ice cold solution of hydrochloric acid (70 μL, 4 M in dioxane) was added to Example 53B (61 mg) and the reaction was allowed to stir at room temperature for 25 minutes. The reaction mixture was quenched with saturated sodium bicarbonate and extracted with dichloromethane three times. The combined organic layers were dried over anhydrous sodium sulfate, filtered and concentrated. The residue was purified by RP-HPLC on a Gilson PLC 2020 using a Luna™ column (250×50 mm, 10 mm) (5-75% over 30 minutes with acetonitrile in water containing 0.01% trifluoroacetic acid). The desired fractions were combined, washed with saturated sodium bicarbonate and extracted with dichloromethane three times. The organic layers were dried over anhydrous sodium sulfate, filtered and concentrated to give the title compound.

### Example 53D

### (7R,16R)-19,23-dichloro-1-(4-fluorophenyl)-20,22-dimethyl-16-[(4-methylpiperazin-1-yl)methyl]-10-({2-[4-(2,5,8,11-tetraoxatetradecan-14-yl) piperazin-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 53C (26 mg) and 2,5,8,11-tetraoxatetradecan-14-al (7 mg) in dichloromethane (270 μL) at room temperature was added sodium triacetoxyborohydride (8.4 mg), and the reaction was allowed to stir for 4 hours. The reaction mixture was quenched with saturated sodium bicarbonate and extracted with dichloromethane three times. The combined organic layers were dried over anhydrous sodium sulfate, filtered and concentrated to give the title compound that was used without further purification.

### Example 53E

### tert-butyl (7R,16R)-19,23-dichloro-1-(4-fluorophenyl)-20,22-dimethyl-16-[(4-methylpiperazin-1-yl)methyl]-10-({2-[4-(2,5,8,11-tetraoxatetradecan-14-yl) piperazin-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 53D (32 mg) in dichloromethane (130 μL) was added trifluoroacetic acid (130 μL), and the reaction was allowed to stir for 4 hours. The reaction was concentrated under a stream of nitrogen and taken up in water and acetonitrile. The mixture was purified by RP-HPLC on a Gilson PLC 2020 using a Luna™ column (250×50 mm, 10 mm) (5-85% over 30 minutes with acetonitrile in water containing 10 mM ammonium acetate) to give the title compound. ¹H NMR (400 MHz, dimethylsulfoxide-d₆) δ ppm 8.73 (s, 1H), 8.31 (d, 1H), 7.26-7.07 (m, 5H), 6.79 (d, 1H), 6.75-6.66 (m, 2H), 6.25-6.15 (m, 1H), 5.84-5.76 (m, 1H), 5.03-4.79 (m, 3H), 4.50-4.35 (m, 2H), 3.75-3.65 (m, 2H), 3.62-3.35 (m, 14H), 3.23 (s, 3H), 2.98-2.87 (m, 1H), 2.76-2.59 (m, 2H), 2.47-2.29 (m, 10H), 2.23 (s, 3H), 2.02-1.93 (m, 6H), 1.75-1.61 (m, 2H). MS (ESI) m/z 1131.1 (M−H)⁻.

### Example 54

### (7R,16R)-19,23-dichloro-1-(4-fluorophenyl)-10-[(2-{4-[(2,5,8,11,14,17,20-heptaoxadocosan-22-yl)oxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 54A

### 2-(4-((2,5,8,11,14,17,20-heptaoxadocosan-22-yl)oxy)phenyl)-4,4,5,5-tetramethyl-1,3,2-dioxaborolane

4-(4,4,5,5-Tetramethyl-1,3,2-dioxaborolan-2-yl)phenol (250 mg) and 2,5,8,11,14,17,20-heptaoxadocosan-22-yl benzenesulfonate (655 mg) were dissolved in N,N-dimethylformamide (6 mL). Cesium carbonate (740 mg) was added, and the solution was heated to 85° C. overnight. The solution was cooled, added to water (18 mL) and extracted with ethyl acetate (15 mL) three times. The extracts were combined, washed with brine (5 mL) and dried over anhydrous sodium sulfate. The solution was filtered, and concentrated, and the residue was purified by flash column chromatography on silica gel using a gradient of 50-100% ethyl acetate in heptanes. The solvent was removed under vacuum to yield the title compound. ¹H NMR (500 MHz, dimethylsulfoxide-d₆) δ ppm 7.60 (d, 2H), 6.93 (d, 2H), 4.11 (m, 2H), 3.74 (m, 2H), 3.60-3.57 (m, 2H), 3.55-3.48 (m, 20H), 3.43-3.40 (m, 2H), 3.23 (s, 3H), 1.27 (s, 12H). MS (ESI) m/z 560.4 (M+NH₄)⁺.

### Example 54B

### (2-(4-((2,5,8,11,14,17,20-heptaoxadocosan-22-yl)oxy)phenyl)pyrimidin-4-yl)methanol

The title compound was prepared by substituting Example 54A for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.81 (d, 1H), 8.33 (d, 2H), 7.41 (d, 1H), 7.06 (d, 2H), 5.63 (t, 1H), 4.61 (d, 2H), 4.17 (t, 2H), 3.78 (t, 2H), 3.58 (m, 4H), 3.52-3.47 (m, 18H), 3.43-3.40 (m, 2H), 3.29 (s, 3H). MS (ESI) m/z 525.4 (M+H)⁺.

### Example 54C

### (7R,16R)-19,23-dichloro-1-(4-fluorophenyl)-10-[(2-{4-[(2,5,8,11,14,17,20-heptaoxadocosan-22-yl)oxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 54B for Example 38D in Example 38E. ¹H NMR (500 MHz, dimethylsulfoxide-d₆) δ ppm 8.82 (d, 1H), 8.72 (s, 1H), 8.34 (d, 2H), 7.46 (d, 1H), 7.23-7.11 (m, 4H), 7.07 (d, 2H), 6.87 (d, 1H), 6.73 (dd, 1H), 6.20 (m, 1H), 5.84 (s, 1H), 5.20 (q, 2H), 4.88 (m, 1H), 4.45 (m, 2H), 4.18 (t, 2H), 3.78 (t, 2H), 3.65 (d, 1H), 3.58 (m, 4H), 3.54-3.47 (m, 18H), 3.43-3.38 (m, 2H), 3.22 (s, 3H), 2.98 (d, 2H), 2.67 (m, 3H), 2.45 (m, 2H), 2.35 (m, 4H), 2.15 (s, 3H), 1.97 (s, 6H). MS (ESI) m/z 1259.2 (M+H)⁺.

### Example 55

### (7R,16R)-19-chloro-10-[(2-{(2R)-1-[3-(dimethylphosphoryl)propanoyl]pyrrolidin-2-yl}pyrimidin-4-yl)methoxy]-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 55A

### (R)-3-(dimethylphosphoryl)-1-(2-(4-(hydroxymethyl)pyrimidin-2-yl) pyrrolidin-1-yl) propan-1-one

To 3-(dimethylphosphoryl)propanoic acid (305 mg) in dimethylformamide (8 mL), was added O-(7-azabenzotriazol-1-yl)-N,N,N′,N′-tetramethyluronium hexafluorophosphate (770 mg) and N-ethyl-N-isopropylpropan-2-amine (1050 μL). The reaction was stirred for 3 minutes, and added to a solution of Example 43F (364 mg) and N-ethyl-N-isopropylpropan-2-amine (900 μL) in dimethylformamide (8 mL). The combined mixture was stirred for 1 hour. The mixture was diluted with 5 mL water, and chromatographed on a Grace Revelris system using a Luna™ 250×50 mm column, 0-20% acetonitrile in 0.1% aqueous trifluoroacetic acid over 30 minutes to give the title compound. MS (ESI) m/z 312.1 (M+H)⁺.

### Example 55B

### (R)-(2-(1-(3-(dimethylphosphoryl)propanoyl) pyrrolidin-2-yl)pyrimidin-4-yl)methyl methanesulfonate

To Example 55A (115 mg) in dichloromethane (1.8 mL) cooled in an ice-water bath was added triethylamine (105 μL). The reaction was stirred for 15 minutes, and methanesulfonyl chloride (60 μL) was added dropwise. The reaction was stirred at room temperature for 1 hour. Aqueous sodium carbonate solution (0.4 mL, 2M) was added, and the reaction was stirred for 15 minutes. Sodium sulfate was added and the reaction was stirred for 20 minutes. The mixture was filtered and concentrated to give the title compound which was used in the next step without further purification. MS (ESI) m/z 390.1 (M+H)⁺.

### Example 55C

### tert-butyl (7R,16R)-19-chloro-10-[(2-{(2R)-1-[3-(dimethylphosphoryl)propanoyl]pyrrolidin-2-yl}pyrimidin-4-yl)methoxy]-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 55B for Example 43H in Example 431. MS (ESI) m/z 1054.5 (M+H)⁺.

### Example 55D

### (7R,16R)-19-chloro-10-[(2-{(2R)-1-[3-(dimethylphosphoryl)propanoyl]pyrrolidin-2-yl}pyrimidin-4-yl)methoxy]-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 55C for Example 431 in Example 43J. ¹H NMR (400 MHz, dimethylsulfoxide-d₆) δ ppm 8.83 (d, 1H), 8.53 (s, 1H), 7.49 (dd, 1H), 7.04 (m, 4H), 6.97 (dd, 1H), 6.86 (dd, 1H), 6.521 (s, 2H), 6.17 (d, 1H), 5.81 (m, 1H), 5.34 (s, 2H), 4.98 (m, 1H), 4.74 (m, 1H), 4.66 (m, 2H), 4.13 (m, 2H), 3.63 (m, 2H), 3.54 (m, 1H), 3.43 (m, 1H), 3.04 (m, 2H), 2.83 (m, 4H), 2.56 (m, 1H), 2.43 (s, 3H), 2.34 (m, 2H), 2.02 (s, 3H), 1.89 (m, 2H), 1.74 (m, 2H), 1.29 (m, 4H), 1.19 (m, 2H). MS (ESI) m/z 998.6 (M+H)⁺.

### Example 56

### (7R,16R)-19,23-dichloro-10-({2-[(3S,4S)-3,4-dihydroxypyrrolidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 56A

### (2-((3S,4S)-3,4-bis((tert-butyldimethylsilyl)oxy) pyrrolidin-1-yl)pyrimidin-4-yl)methanol

(2-Chloropyrimidin-4-yl)methanol (42 mg), (3S,4S)-3,4-bis((tert-butyldimethylsilyl)oxy) pyrrolidine (100 mg) and triethylamine (88 mg) were dissolved in acetonitrile (2 mL). The solution was heated to 80° C. for five hours and cooled. The solution was concentrated, and the residue was purified by flash column chromatography on silica gel, using a gradient of 10-50% ethyl acetate in heptanes. The solvent was removed under vacuum to yield the title compound. MS (ESI) m/z 440.2 (M+H)⁺.

### Example 56B

### (7R,16R)-19,23-dichloro-10-({2-[(3S,4S)-3,4-dihydroxypyrrolidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 56A for Example 38D in Example 38E. ¹H NMR (500 MHz, dimethylsulfoxide-d₆) δ ppm 8.81 (s, 1H), 8.38 (d, 1H), 7.31-7.21 (m, 4H), 6.90 (d, 1H), 6.83 (dd, 1H), 6.77 (d, 1H), 6.28 (m, 1H), 5.92 (bs, 1H), 5.20 (s, 2H), 5.02 (q, 2H), 4.53 (m, 2H), 4.43 (m, 2H), 4.11 (d, 2H), 3.70-3.62 (m, 4H), 3.56-3.48 (m, 2H), 3.04 (d, 2H), 2.77 (m, 3H), 2.56 (m, 2H), 2.46 (m, 2H), 2.28 (s, 3H), 2.07 (s, 3H), 2.06 (s, 3H). MS (ESI) m/z 946.3 (M+H)⁺.

### Example 57

### (7R,16R)-19,23-dichloro-1-(4-fluorophenyl)-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 57A

### 4-(((tert-butyldiphenylsilyl)oxy)methyl)-2-chloropyrimidine

To a solution of (2-chloropyrimidin-4-yl)methanol (3.8 g) and tert-butylchlorodiphenylsilane (7.23 g) in dimethylformamide (30 mL) was added imidazole (3.58 g). The mixture was stirred under nitrogen at room temperature overnight. The mixture was diluted with water (50 mL), ethyl acetate (400 mL). The organic layer was separated and washed with water and brine and dried over sodium sulfate. Filtration and evaporation of the solvent gave crude product which was loaded on a Redi-Sep Gold 220 g column and eluted with 20% ethyl acetate in heptane to give the title compound. MS (ESI) m/z 383.2 (M+H)⁺.

### Example 57B

### 4-(((tert-butyldiphenylsilyl)oxy)methyl)-2-(1,4-dioxaspiro[4.5]dec-7-en-8-yl)pyrimidine

To a solution of 4,4,5,5-tetramethyl-2-(1,4-dioxaspiro[4.5]dec-7-en-8-yl)-1,3,2-dioxaborolane (7.30 g) and Example 57A (10.5 g) in tetrahydrofuran (120 mL) was added Pd(Ph₃P)₄ (1.58 g) and aqueous saturated sodium bicarbonate (60 mL). The mixture was stirred under nitrogen at 70° C. overnight. The mixture was concentrated under vacuum and the residue was diluted with water (120 mL) and ethyl acetate (600 mL). The organic layer was separated and washed with water and brine and dried over sodium sulfate. Filtration and evaporation of the solvent gave crude product which was loaded on a Redi-Sep Gold 220 g column and eluted with 20% ethyl acetate in heptane to give the title compound. MS (ESI) m/z 487.2 (M+H)⁺.

### Example 57C

### 4-(((tert-butyldiphenylsilyl)oxy)methyl)-2-(1,4-dioxaspiro[4.5]decan-8-yl)pyrimidine

To a solution of Example 57B (10 g) in tetrahydrofuran (120 mL) was added Pd/C (10% 1.5 g). The mixture was stirred under hydrogen (25 psi) at room temperature for 4 hours. The mixture was filtered and concentrated under vacuum to give the title compound. MS (ESI) m/z 489.2 (M+H)⁺.

### Example 57D

### 4-(4-(((tert-butyldiphenylsilyl)oxy)methyl)pyrimidin-2-yl)cyclohexanone

To a solution of Example 57C (10 g) in acetone (70 mL) and water (30 mL) was added pyridinium p-toluenesulfonate (1.5 g). The mixture was stirred at reflux for 16 hours. The mixture was concentrated under vacuum and the residue was diluted with water (120 mL) and ethyl acetate (600 mL). The organic layer was separated and washed with water and brine and dried over sodium sulfate. Filtration and evaporation of the solvent gave crude product which was loaded on a Redi-Sep Gold 220 g column and eluted with 20% ethyl acetate in heptane to give the title compound. MS (ESI) m/z 445.3 (M+H)⁺.

### Example 57E

### (1r,4r)-4-(4-(((tert-butyldiphenylsilyl)oxy)methyl)pyrimidin-2-yl)cyclohexanol

To a solution of Example 57D (2.2 g) in tetrahydrofuran (20 mL) was added sodium borohydride (0.56 g). The mixture was stirred at room temperature for 3 hours. The mixture was diluted with water (20 mL) and ethyl acetate (300 mL). The organic layer was separated and washed with water and brine and dried over sodium sulfate. Filtration and evaporation of the solvent gave crude product which was loaded on a Redi-Sep Gold 120 g column and eluted with 40% ethyl acetate in heptane to give the title compound. MS (ESI) m/z 447.3 (M+H)⁺.

### Example 57F

### 4-(((tert-butyldiphenylsilyl)oxy)methyl)-2-((1r,4r)-4-(2-(2-(2-methoxyethoxy)ethoxy)ethoxy)cyclohexyl)pyrimidine

To a suspension of NaH (60% oil dispersion, 120 mg) in tetrahydrofuran (5 mL), a solution of Example 57E (135 mg) in tetrahydrofuran (4 mL) was added dropwise at room temperature and the resulting suspension was stirred at room temperature for 1 hour. To the mixture, tetra-n-butylammonium bromide (13 mg) and 1-bromo-2-(2-(2-methoxyethoxy)ethoxy) ethane (206 mg) were added. The mixture was stirred for two days at 60° C. The mixture was quenched with aqueous ammonium chloride, extracted with ethyl acetate (300 mL), washed with water and brine and dried over sodium sulfate. Filtration and evaporation of the solvent gave the crude product which was loaded on a Redi-Sep Gold 40 g column and eluted with 5% methanol in dichloromethane to give the title compound. MS (ESI) m/z 593.5 (M+H)⁺.

### Example 57G

### (2-((1r,4r)-4-(2-(2-(2-methoxyethoxy)ethoxy)ethoxy)cyclohexyl)pyrimidin-4-yl)methanol

To a solution of Example 57F (110 mg) in tetrahydrofuran (10 mL) was added cesium fluoride (300 mg) and methanol (5 mL). The mixture was stirred at room temperature overnight. The solvent was evaporated under vacuum and the residue was triturated with heptane (30 mL) and with dichloromethane (30 mL). Evaporation of the solvent gave the title compound. MS (ESI) m/z 355.4 (M+H)⁺.

### Example 57H

### tert-butyl (7R,16R)-19,23-dichloro-1-(4-fluorophenyl)-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a 4 mL vial containing Example 16N (50 mg), Example 57G (23 mg) and triphenylphosphine (52.5 mg) was added toluene (500 μL) and tetrahydrofuran (500 μL) followed by (E)-N¹,N¹,N²,N²-tetramethyldiazene-1,2-dicarboxamide (34.5 mg). The mixture was purged with argon for 3 minutes and was stirred at 50° C. for 4 hours. The mixture was diluted with dichloromethane (10 mL) and loaded on a Redi-Sep Gold 40 g column and eluted with 30% ethyl acetate in heptane (1 L) followed by 5% 7N ammonium in methanol in dichloromethane (1 L) to give the title compound. MS (ESI) m/z 1147.3 (M+H)⁺.

### Example 57I

### (7R,16R)-19,23-dichloro-1-(4-fluorophenyl)-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 57H (76 mg) in dichloromethane (3 mL) was added trifluoroacetic acid (3 mL). The mixture was stirred at room temperature for 6 hours. The mixture was concentrated under vacuum and the residue was dissolved in dimethylformamide (3 mL) and loaded on HPLC (Gilson 2020 system. Luna™ C-18, 250×50 mm column, mobile phase A: 0.1% trifluoroacetic acid in water; B: acetonitrile: 20-75% B to A gradient at 70 mL/minute in 35 minutes) to afford the title compound. ¹H NMR (400 MHz, dimethylsulfoxide-d₆) δ ppm 8.70-8.63 (m, 2H), 7.39 (d, 1H), 7.16 (dd, 2H), 7.14-7.06 (m, 2H), 6.79 (d, 1H), 6.69 (dd, 1H), 6.16 (dd, 1H), 5.79 (d, 1H), 5.09 (d, 1H), 5.01 (d, 1H), 4.88-4.79 (m, 1H), 4.40 (d, 2H), 3.60-3.50 (m, 1H), 3.54-3.44 (m, 10H), 3.40 (dd, 2H), 3.20 (s. 3H), 2.90 (d, 1H), 2.80-2.56 (m, 3H), 2.42 (s, 2H), 2.36 (s, 6H), 2.16 (s, 3H), 2.03 (dd, 2H), 1.93 (d, 8H), 1.57 (qd, 2H), 1.24 (dt, 2H). MS (ESI) m/z 1089.5 (M+H)⁺.

### Example 58

### (7R,16R)-19,23-dichloro-1-(4-fluorophenyl)-20,22-dimethyl-16-[(4-methylpiperazin-1-yl)methyl]-10-({2-[1-(2,5,8,11,14-pentaoxapentadecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 58A

### 2-(1-(2,5,8,11,14-pentaoxapentadecyl)cyclobutyl)-4-(dimethoxymethyl)pyrimidine

Example 58A was synthesized according to the procedure described for Example 44F, substituting 13-bromo-2,5,8,11-tetraoxatridecane for Example 44E. MS (APCI) m/z 429.4 (M+H)⁺.

### Example 58B

### 2-(1-(2,5,8,11,14-pentaoxapentadecyl)cyclobutyl)pyrimidine-4-carbaldehyde

Example 58B was synthesized according to the procedure described for Example 29G, substituting Example 58A for Example 29F. MS (APCI) m/z 383.4 (M+H)⁺.

### Example 58C

### (2-(1-(2,5,8,11,14-pentaoxapentadecyl)cyclobutyl)pyrimidin-4-yl)methanol

Example 58C was synthesized according to the procedure described for Example 29H, substituting Example 58B for Example 29G. MS (APCI) m/z 385.4 (M+H)⁺.

### Example 58D

### tert-butyl (7R,16R)-19,23-dichloro-1-(4-fluorophenyl)-20,22-dimethyl-16-[(4-methylpiperazin-1-yl)methyl]-10-({2-[1-(2,5,8,11,14-pentaoxapentadecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

Example 58D was synthesized according to the procedure described for Example 291, substituting Example 58C for Example 29H. MS (APCI) m/z 1175.4 (M+H)⁺.

### Example 58E

### (7R,16R)-19,23-dichloro-1-(4-fluorophenyl)-20,22-dimethyl-16-[(4-methylpiperazin-1-yl)methyl]-10-({2-[1-(2,5,8,11,14-pentaoxapentadecan-1-yl)cyclobutyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 58E was synthesized according to the procedure described for Example 29J, substituting Example 58D for Example 291. ¹H NMR (500 MHz, dimethylsulfoxide-d₆) δ ppm 8.75 (d, 1H), 8.74 (s, 1H), 7.42 (d, 1H), 7.27-7.08 (m, 4H), 6.89 (d, 1H), 6.76 (dd, 1H), 6.25 (dd, 1H), 5.80 (d, 1H), 5.11 (q, 2H), 4.88 (d, 1H), 4.45 (d, 2H), 3.86 (s, 2H), 3.62 (dd, 1H), 3.51-3.38 (m, 16H), 3.21 (s, 3H), 3.01-2.92 (m, 1H), 2.79-2.64 (m, 2H), 2.57 (s, 8H), 2.48-2.41 (m, 2H), 2.32 (s, 3H), 2.21-2.09 (m, 2H), 2.05-1.91 (m, 7H), 1.86-1.71 (m, 1H). MS (APCI) m/z 1120.6 (M+H)⁺.

### Example 59

### (7R,16R)-19,23-dichloro-1-(4-fluorophenyl)-10-({2-[1-(2,5,8,11,14,17-hexaoxaoctadecan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 59A

### 2-(1-(2,5,8,11,14,17-hexaoxaoctadecyl)cyclobutyl)-4-(dimethoxymethyl)pyrimidine

Example 59A was synthesized according to the procedure described for Example 44F, substituting 16-bromo-2,5,8,11,14-pentaoxahexadecane for Example 44E. (APCI) m/z 473.4 (M+H)⁺.

### Example 59B

### 2-(1-(2,5,8,11,14,17-hexaoxaoctadecyl)cyclobutyl)pyrimidine-4-carbaldehyde

Example 59B was synthesized according to the procedure described for Example 29G, substituting Example 59A for Example 29F. (APCI) m/z 327.4 (M+H)⁺.

### Example 59C

### (2-(1-(2,5,8,11,14,17-hexaoxaoctadecyl)cyclobutyl)pyrimidin-4-yl)methanol

Example 59C was synthesized according to the procedure described for Example 29H, substituting Example 59B for Example 29G. (APCI) m/z 429.4 (M+H)⁺.

### Example 59D

### tert-butyl (7R,16R)-19,23-dichloro-1-(4-fluorophenyl)-10-({2-[1-(2,5,8,11,14,17-hexaoxaoctadecan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 59D was synthesized according to the procedure described for Example 291, substituting Example 59C for Example 29H. MS (APCI) m/z 1175.4 (M+H)⁺.

### Example 59E

### (7R,16R)-19,23-dichloro-1-(4-fluorophenyl)-10-({2-[1-(2,5,8,11,14,17-hexaoxaoctadecan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 59E was synthesized according to the procedure described for Example 29J, substituting Example 59D for Example 291. ¹H NMR (500 MHz, dimethylsulfoxide-d₆) δ ppm 8.75 (d, 1H), 8.74 (s, 1H), 7.42 (d, 1H), 7.26-7.09 (m, 4H), 6.89 (d, 1H), 6.76 (dd, 1H), 6.25 (dd, 1H), 5.80 (d, 1H), 5.11 (q, 2H), 4.88 (p, 1H), 4.45 (d, 2H), 3.86 (s, 2H), 3.62 (dd, 1H), 3.51-3.38 (m, 20H), 3.22 (s, 3H), 3.02-2.92 (m, 1H), 2.77-2.65 (m, 2H), 2.65-2.54 (m, 8H), 2.48-2.41 (m, 2H), 2.34 (s, 3H), 2.20-2.08 (m, 2H), 2.03-1.90 (m, 7H), 1.85-1.72 (m, 1H). MS (APCI) m/z 1163.5 (M+H)⁺.

### Example 60

### (7R,16R)-19,23-dichloro-1-(4-fluorophenyl)-10-({2-[1-(2,5,8,11,14,17,20-heptaoxahenicosan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 60A

### 2-(1-(2,5,8,11,14,17,20-heptaoxahenicosyl)cyclobutyl)-4-(dimethoxymethyl)pyrimidine

Example 60A was synthesized according to the procedure described for Example 44F, substituting 19-bromo-2,5,8,11,14,17-hexaoxanonadecane for Example 44E. MS (APCI) m/z 517.4 (M+H)⁺.

### Example 60B

### 2-(1-(2,5,8,11,14,17,20-heptaoxahenicosyl)cyclobutyl)pyrimidine-4-carbaldehyde

Example 60B was synthesized according to the procedure described for Example 29G, substituting Example 60A for Example 29F. MS (APCI) m/z 471.4 (M+H)⁺.

### Example 60C

### (2-(1-(2,5,8,11,14,17,20-heptaoxahenicosyl)cyclobutyl)pyrimidin-4-yl)methanol

Example 60C was synthesized according to the procedure described for Example 29H, substituting Example 60B for Example 29G. MS (APCI) m/z 473.4 (M+H)⁺.

### Example 60D

### tert-butyl (7R,16R)-19,23-dichloro-1-(4-fluorophenyl)-10-({2-[1-(2,5,8,11,14,17,20-heptaoxahenicosan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 60D was synthesized according to the procedure described for Example 291, substituting Example 60C for Example 29H. MS (APCI) m/z 1131.7 (M+H)⁺.

### Example 60E

### (7R,16R)-19,23-dichloro-1-(4-fluorophenyl)-10-({2-[1-(2,5,8,11,14,17,20-heptaoxahenicosan-1-yl)cyclobutyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 60E was synthesized according to the procedure described for Example 29J, substituting Example 60D for Example 291. ¹H NMR (500 MHz, dimethylsulfoxide-d₆) δ ppm 8.75 (d, 1H), 8.73 (s, 1H), 7.43 (d, 1H), 7.27-7.09 (m, 4H), 6.88 (d, 1H), 6.74 (dd, 1H), 6.24 (dd, 1H), 5.82 (d, 1H), 5.11 (q, 2H), 4.88 (p, 1H), 4.44 (d, 2H), 3.86 (s, 2H), 3.61 (dd, 1H), 3.51-3.38 (m, 24H), 3.22 (s, 3H), 3.00-2.91 (m, 1H), 2.75-2.61 (m, 2H), 2.57-2.42 (m, 10H), 2.24 (s, 3H), 2.20-2.09 (m, 2H), 2.04-1.90 (m, 7H), 1.87-1.71 (m, 1H). MS (APCI) m/z 1207.4 (M+H)⁺.

### Example 61

### (7R,16R)-19,23-dichloro-1-(4-fluorophenyl)-20,22-dimethyl-16-[(4-methylpiperazin-1-yl)methyl]-10-{[2-(1-oxo-2,9-diazaspiro[5.5]undecan-9-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 61A

### 9-(4-(hydroxymethyl)pyrimidin-2-yl)-2,9-diazaspiro[5.5]undecan-1-one

A solution of 2,9-diazaspiro[5.5]undecan-1-one, hydrochloric acid (260 mg), (2-chloropyrimidin-4-yl)methanol (150 mg) and N,N-diisopropylethylamine (910 μL) in acetonitrile (2.6 mL) was heated to 80° C. for 3 hours and stirred overnight at room temperature. The reaction was diluted with water and extracted with ethyl acetate three times. The combined organic layers were dried over anhydrous sodium sulfate, filtered and concentrated. The residue was purified by normal phase MPLC on a Teledyne Isco CombiFlash® Rf+12 g gold silica gel column eluting with 0-7.5% methanol in dichloromethane to give the title compound. ¹H NMR (400 MHz, dimethylsulfoxide-d₆) δ ppm 8.31 (d, 1H), 7.31 (br s, 1H), 6.67 (d, 1H), 5.42-5.30 (m, 1H), 4.41-4.22 (m, 4H), 3.30-3.20 (m, 2H), 3.16-3.06 (m, 2H), 1.95-1.64 (m, 6H), 1.47-1.34 (m, 2H).

### Example 61B

### tert-butyl (7R,16R)-19,23-dichloro-1-(4-fluorophenyl)-20,22-dimethyl-16-[(4-methylpiperazin-1-yl)methyl]-10-{[2-(1-oxo-2,9-diazaspiro[5,5]undecan-9-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (35 mg) and Example 61A (18 mg) in toluene (110 μL) and tetrahydrofuran (110 μL) was added triphenylphosphine (34 mg) followed by N,N,N′,N′-tetramethylazodicarboxamide (22 mg), and the reaction was allowed to stir at 50° C. for 4 hours. The reaction was diluted with ethyl acetate, filtered over diatomaceous earth and concentrated. The residue was purified by normal phase MPLC on a Teledyne Isco CombiFlash® Rf+4 g gold silica gel column eluting with 1-10% methanol in dichloromethane to give the title compound. ¹H NMR (400 MHz, dimethylsulfoxide-d₆) δ ppm 8.74 (s, 1H), 8.34 (d, 1H), 7.35-7.29 (m, 1H), 7.27-7.13 (m, 5H), 6.92-6.78 (m, 2H), 6.69 (d, 1H), 6.03 (dd, 1H), 5.66 (d, 1H), 5.04-4.82 (m, 2H), 4.80-4.69 (m, 1H), 4.53-4.36 (m, 2H), 4.35-4.23 (m, 2H), 3.70-3.58 (m, 1H), 3.18-3.08 (m, 2H), 2.92-2.59 (m, 4H), 2.44-2.20 (m, 4H), 2.14 (s, 3H), 2.09 (s, 3H), 1.96-1.65 (m, 9H), 1.48-1.36 (m, 2H), 1.07 (s, 9H).

### Example 61C

### (7R,16R)-19,23-dichloro-1-(4-fluorophenyl)-20,22-dimethyl-16-[(4-methylpiperazin-1-yl)methyl]-10-{[2-(1-oxo-2,9-diazaspiro[5,5]undecan-9-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 61B (38 mg) in dichloromethane (180 μL) was added trifluoroacetic acid (180 μL), and the reaction was allowed to stir for 4 hours. The reaction was concentrated under a stream of nitrogen and taken up in water and acetonitrile. The mixture was purified by RP-HPLC on a Gilson PLC 2020 using a Luna™ column (250×50 mm, 10 mm) (5-85% over 30 minutes with acetonitrile in water containing 10 mM ammonium acetate) to give the title compound. ¹H NMR (500 MHz, dimethylsulfoxide-d₆) δ ppm 8.71 (s, 1H), 8.30 (d, 1H), 7.35-7.28 (m, 1H), 7.23-7.08 (m, 5H), 6.78 (d, 1H), 6.75-6.66 (m, 2H), 6.23-6.14 (m, 1H), 5.88-5.80 (m, 1H), 5.01-4.83 (m, 3H), 4.50-4.38 (m, 2H), 4.34-4.22 (m, 2H), 3.60-3.51 (m, 1H), 3.36-3.25 (m, 2H), 3.16-3.07 (m, 2H), 2.97-2.88 (m, 1H), 2.75-2.59 (m, 2H), 2.44 (br s, 4H), 2.22 (s, 3H), 1.98 (s, 3H), 1.95 (s, 3H), 1.92-1.83 (m, 2H), 1.82-1.74 (m, 2H), 1.73-1.65 (m, 2H), 1.47-1.36 (m, 2H). MS (ESI) m/z 1009.0 (M−H)⁻.

### Example 62

### (7R,16R)-19,23-dichloro-10-{[2-(1,3-dihydroxypropan-2-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 62A

### ethyl 2-(oxetan-3-yl)pyrimidine-4-carboxylate

To a solution of oxetane-3-carboximidamide acetic acid (1.8 g) in acetonitrile (35 mL) was added ethyl 4-(dimethylamino)-2-oxobut-3-enoate (2.01 g). Potassium carbonate (6 g) was added and the reaction mixture was stirred for 6 hours at reflux. The reaction mixture was concentrated in vacuo. To the residue water was added and the aqueous phase was extracted with ethyl acetate. The combined organic extracts were washed with brine, dried over magnesium sulfate, filtered and concentrated in vacuo. Purification by chromatography on silica gel using an ISCO CombiFlash® Companion MPLC (25 g Chromabond® SiOH column, eluting with 0-10% dichloromethane/methanol) provided the title compound. MS (ESI) m/z 209.4 (M+H)⁺.

### Example 62B

### (2-(oxetan-3-yl)pyrimidin-4-yl)methanol

To a solution of Example 62A (530 mg) in methanol (25 mL) was added NaBH₄ (200 mg) and the reaction mixture was stirred for 2 hours at ambient temperature. The reaction mixture was concentrated in vacuo. To the residue was added water (10 mL). The aqueous phase was purified using a Chromabond® RP C 18 column (gradient 5-30% acetonitrile in water). The desired fractions were combined and concentrated in vacuo. To the residue was added dichloromethane. The material was filtered off and washed twice with dichloromethane (10 mL). The combined organic phases were concentrated in vacuo. Purification by chromatography on silica gel using an ISCO CombiFlash® Companion MPLC (15 g Chromabond® SiOH column, eluting with 0-10% dichloromethane/methanol) provided title compound. MS (ESI) m/z 167.4 (M+H)⁺.

### Example 62C

### tert-butyl (4R,9R)-13,15-dichloro-26-(4-fluorophenyl)-12,16-dimethyl-9-((4-methylpiperazin-1-yl)methyl)-66-((2-(oxetan-3-yl)pyrimidin-4-yl)methoxy)-3,7,10-trioxa-2(5,4)-thieno[2,3-d]pyrimidina-1(1,4),6(1,3)-dibenzenacyclodecaphane-4-carboxylate

A 4 mL vial, equipped with stir bar, was charged with Example 16N (50 mg), Example 62B (15 mg), triphenylphosphine (25 mg) and di-tert-butyl azodicarboxylate (23 mg) and was purged for 10 minutes with nitrogen. Toluene (1.0 mL) was added and the reaction mixture was stirred for 24 hours at room temperature and for 4 hours at 50° C. To the reaction mixture was added Telos bulk sorbents and the mixture was concentrated in vacuo. The residue was purified by normal phase MPLC on a Teledyne-Isco-CombiFlash® system (eluting 0-10% methanol in dichloromethane) to afford the title compound. MS (ESI) m/z 957.4 (M+H)⁺.

### Example 62D

### (7R,16R)-19,23-dichloro-10-{[2-(1,3-dihydroxypropan-2-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

To a solution of Example 62C (46 mg) in dichloromethane (217 μL) was added trifluoroacetic acid (222 μL). The reaction mixture was stirred for 6 hours at ambient temperature. The reaction mixture was concentrated in vacuo and stored in a freezer overnight. To the residue was added to cold aqueous sodium bicarbonate solution (5%) and the mixture was extracted twice with dichloromethane. The combined organic phases were dried via DryDisk® and concentrated in vacuo. The residue was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, MeOD) δ ppm 8.71 (d, 1H), 8.59 (s, 1H), 7.64 (d, 1H), 7.12 (m, 2H), 7.00 (m, 2H), 6.74 (d, 1H), 6.67 (m, 1H), 6.14 (m, 1H), 6.09 (d, 1H), 5.13 (m, 3H), 4.52 (m, 1H), 4.35 (m, 1H), 4.05-3.95 (m, 4H), 3.65 (m, 1H), 3.33 (m, 1H), 3.09 (m, 1H), 2.85-2.65 (m, 10H), 2.57 (s, 3H), 2.13 (s, 3H), 1.99 (s, 3H). MS (ESI) m/z 919.1 (M+H)⁺.

### Example 63

### (7R,16R)-19,23-dichloro-1-(4-fluorophenyl)-20,22-dimethyl-16-[(4-methylpiperazin-1-yl)methyl]-10-[(2-{1-[(2,5,8,11,14-pentaoxahexadecan-16-yl)oxy]cyclobutyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 63A

### (2-iodopyrimidin-4-yl)methanol

Hydrogen iodide (22.37 mL) cooled to about −5° C. with an ice-salt bath, was added portionwise to (2-chloropyrimidin-4-yl)methanol (4.3 g) at 0° C. in a 100 mL flask for 1 hour. A quench was performed with sodium carbonate followed by concentrated sodium hydroxide solution until the pH reached 9. The mixture was poured into dichloromethane. The organic layer was separated, washed with sodium thiosulfate solution, dried over sodium sulfate, filtered and concentrated to provide the title compound which was contaminated with 5% starting chloride. ¹H NMR (400 MHz, dimethylsulfoxide-d₆) δ ppm 8.51 (d, 1H), 7.87 (d, 1H), 5.70 (t, 1H), 4.53 (d, 2H). MS (ESI) m/z 237.0 (M+H)⁺.

### Example 63B

### 4-(((tert-butyldimethylsilyl)oxy)methyl)-2-iodopyrimidine

To a solution of Example 63A (4 g) in 100 mL dichloromethane at 0° C. was added 2,6-lutidine (2.96 mL) and tert-butyldimethylsilyl trifluoromethanesulfonate (4.28 mL). The reaction was stirred for 20 minutes. The mixture was diluted with ethyl acetate, washed with water and brine, dried over sodium sulfate, filtered and concentrated. The crude product was purified by silica gel chromatography using 1% ethyl acetate in heptanes as eluent to provide the title compound. ¹H NMR (400 MHz, dimethylsulfoxide-d₆) δ ppm 8.54 (dd, 1H), 7.52 (d, 1H), 4.71 (s, 2H), 0.92 (s, 9H), 0.10 (s, 6H).

### Example 63C

### 1-(4-(((tert-butyldimethylsilyl)oxy)methyl)pyrimidin-2-yl)cyclobutanol

N-Butyllithium (6.03 mL, 2.5 M in hexanes) was added to Example 63B (4.4 g) in 50 mL tetrahydrofuran at −78° C. Cyclobutanone (3.52 g) was added 10 seconds after, and the reaction was stirred for 1 hour, while warming to room temperature. The mixture was poured into ethyl acetate and washed with pH 7 buffer and brine, and concentrated. The crude material was chromatographed on silica gel using 2-25% ethyl acetate in heptanes as eluent to give the title compound. ¹H NMR (400 MHz, dimethylsulfoxide-d₆) δ ppm 8.81 (d, 1H), 7.38 (d, 1H), 5.45 (s, 1H), 4.76 (s, 2H), 2.56 (m, 2H), 2.23 (m, 2H), 1.84 (m, 1H), 1.72 (m, 1H), 0.92 (s, 9H), 0.10 (s, 6H). MS (ESI) m/z 295.1 (M+H)⁺.

### Example 63D

### (2-(1-(2,5,8,11,14-pentaoxahexadecan-16-yloxy)cyclobutyl)pyrimidin-4-yl)methanol

NaH (32.1 mg, 60% in mineral oil) was added to Example 63C (197 mg) in 5 mL tetrahydrofuran, and the reaction was stirred for 20 minutes. 16-Bromo-2,5,8,11,14-pentaoxahexadecane (253 mg) was added, and the reaction was stirred at 40° C. for 2 hours. The mixture was concentrated and taken up in 23 mL dimethylformamide, and purified by reverse phase chromatography using a 10-75% gradient of acetonitrile in water (with 0.1% ammonium acetate) over 40 minutes on a Grace Reveleris equipped with a Luna™ column: C18(2), 100 Å, 250×50 mm. The fractions containing the desired compound were concentrated. The residue was taken up in 20 mL tetrahydrofuran, tetra-N-butylammonium fluoride (803 μL, 1M in tetrahydrofuran) was added, and the reaction was stirred for 20 minutes and concentrated. The crude material was chromatographed on silica gel using 5-100% ethyl acetate in heptanes as eluent, followed by 10% methanol in ethyl acetate, and then 15% methanol in dichloromethane, to give the title compound. ¹H NMR (400 MHz, dimethylsulfoxide-d₆) δ ppm 8.81 (d, 1H), 7.47 (d, 1H), 5.63 (t, 1H), 4.56 (d, 2H), 3.48 (m, 16H), 3.42 (m, 4H), 3.23 (s, 3H), 2.60 (m, 2H), 2.29 (m, 2H), 1.84 (m, 1H), 1.57 (m, 1H). MS (ESI) m/z 415.2 (M+H)⁺.

### Example 63E

### (7R,16R)-19,23-dichloro-1-(4-fluorophenyl)-20,22-dimethyl-16-[(4-methylpiperazin-1-yl)methyl]-10-[(2-{1-[(2,5,8,11,14-pentaoxahexadecan-16-yl)oxy]cyclobutyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was prepared by substituting Example 63D for Example 38D in Example 38E. ¹H NMR (400 MHz, dimethylsulfoxide-d₆) δ ppm 8.84 (d, 1H), 8.74 (s, 1H), 7.53 (dd, 1H), 7.20 (m, 2H), 7.14 (m, 2H), 6.87 (dd, 1H), 6.76 (dd, 1H), 6.24 (d, 1H), 5.80 (d, 1H), 5.16 (dd, 2H), 4.87 (m, 1H), 4.44 (d, 2H), 3.59 (m, 2H), 3.48 (m, 16H), 3.26 (m, 4H), 3.21 (s, 3H), 2.92 (m, 1H), 2.68 (m, 2H), 2.60 (m, 2H), 2.47 (m, 6H), 2.31 (m, 2H), 2.23 (s, 3H), 1.98 (s, 3H), 1.96 (s, 3H), 1.84 (m, 1H), 1.61 (m, 1H). MS (ESI) m/z 1151.4 (M+H)⁺.

### Example 64

### (7R,16R)-19,23-dichloro-1-(4-fluorophenyl)-20,22-dimethyl-16-[(4-methylpiperazin-1-yl)methyl]-10-[(2-{1-[(2,5,8,11-tetraoxatridecan-13-yl)oxy]cyclobutyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 64A

### (2-(1-(2,5,8,11-tetraoxatridecan-13-yloxy)cyclobutyl)pyrimidin-4-yl)methanol

The title compound was prepared by substituting 13-bromo-2,5,8,11-tetraoxatridecane for 16-bromo-2,5,8,11,14-pentaoxahexadecane in Example 63D. ¹H NMR (400 MHz, dimethylsulfoxide-d₆) δ ppm 8.82 (d, 1H), 7.46 (d, 1H), 5.63 (t, 1H), 4.56 (d, 2H), 3.48 (m, 12H), 3.42 (m, 4H), 3.23 (s, 3H), 2.60 (m, 2H), 2.28 (m, 2H), 1.84 (m, 1H), 1.57 (m, 1H). MS (ESI) m/z 371.2 (M+H)⁺.

### Example 64B

### (7R,16R)-19,23-dichloro-1-(4-fluorophenyl)-20,22-dimethyl-16-[(4-methylpiperazin-1-yl)methyl]-10-[(2-{1-[(2,5,8,11,14-pentaoxahexadecan-16-yl)oxy]cyclobutyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was prepared by substituting Example 64A for Example 38D in Example 38E. ¹H NMR (400 MHz, dimethylsulfoxide-d₆) δ ppm 8.83 (d, 1H), 8.73 (s, 1H), 7.54 (dd, 1H), 7.20 (m, 2H), 7.14 (m, 2H), 6.87 (dd, 1H), 6.74 (dd, 1H), 6.22 (d, 1H), 5.82 (d, 1H), 5.16 (dd, 2H), 4.88 (m, 1H), 4.45 (d, 2H), 3.61 (m, 2H), 3.48 (m, 12H), 3.26 (m, 4H), 3.21 (s, 3H), 2.94 (m, 1H), 2.67 (m, 2H), 2.60 (m, 2H), 2.46 (m, 6H), 2.30 (m, 2H), 2.21 (s, 3H), 1.98 (s, 3H), 1.96 (s, 3H), 1.84 (m, 1H), 1.61 (m, 1H). MS (ESI) m/z 1105.4 (M+H)⁺.

### Example 65

### (7R,16R)-19,23-dichloro-10-({2-[3-(1,3-dihydroxypropan-2-yl)azetidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 65A

### methyl 2-(3-(oxetan-3-yl)azetidin-1-yl)pyrimidine-4-carboxylate

To a solution of 3-(oxetan-3-yl)azetidine (258 mg) in dioxane (10 mL) was added triethylamine (0.70 mL) and the reaction mixture was stirred for 10 minutes at ambient temperature. Subsequently methyl 2-chloropyrimidine-4-carboxylate (300 mg) was added and the reaction mixture was stirred at 80° C. for 3 hours in a Biotage® Initiator microwave unit. To the reaction mixture was added water and the aqueous phase was extracted twice with dichloromethane. The combined organic extracts were washed with brine, dried with sodium sulfate, filtered, and concentrated in vacuo. To the residue was added ethyl acetate and n-heptane. The formed precipitate was filtered off and washed with n-heptane. The precipitate was dried in vacuo at ambient temperature. The crude product was used without any further purification in the next step. MS (APCI) m/z 250.2 (M+H)⁺.

### Example 65B

### (2-(3-(oxetan-3-yl)azetidin-1-yl)pyrimidin-4-yl)methanol

To a solution of Example 65A (286 mg) in methanol (10 mL) was added NaBH₄ (87 mg) at 0° C. The reaction mixture was allowed to warm to ambient temperature and stirring was continued for 70 minutes. NaBH₄ (13 mg) was added again and the reaction mixture was stirred at ambient temperature for 85 minutes. The reaction mixture was concentrated in vacuo. To the residue was added brine and the aqueous phase was extracted twice with dichloromethane. The combined organic extracts were washed with brine, dried via DryDisk® and concentrated in vacuo. The crude product was used without any further purification in the next step. MS (APCI) m/z 222.2 (M+H)⁺.

### Example 65C

### (2-(3-(oxetan-3-yl)azetidin-1-yl)pyrimidin-4-yl)methyl methanesulfonate

Example 65B (50 mg) was dissolved in dichloromethane (2.26 mL) under a nitrogen atmosphere and cooled to 0° C. with iced water. Triethylamine (94 μL) and methanesulfonyl chloride (22.9 μL) were added and the reaction mixture was stirred under cooling for 30 minutes. Brine was added to the reaction mixture and the aqueous layer was extracted with dichloromethane. The combined organic extract were dried over anhydrous magnesium sulfate, filtrated and concentrated in vacuo. The crude product was used without any further purification in the next step. MS (APCI) m/z 300.0 (M+H)⁺.

### Example 65D

### tert-butyl (7R,16R)-19,23-dichloro-10-({2-[3-(1,3-dihydroxypropan-2-yl)azetidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A 4 mL vial, equipped with stir bar, was charged with Example 16N (50 mg) and Example 65C (33 mg). N,N-Dimethylformamide (206 μL) and subsequently cesium carbonate (60.4 mg) were added. The reaction mixture was stirred at ambient temperature for 48 hours. The reaction mixture was added to cold aqueous sodium bicarbonate solution (5%). The precipitate was filtered off after 5 minutes and washed twice with cold water. The precipitate was dried in vacuo overnight at 30° C. MS (ESI) m/z 1012.4 (M+H)⁺.

### Example 65E

### (7R,16R)-19,23-dichloro-10-({2-[3-(1,3-dihydroxypropan-2-yl)azetidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

To a solution of Example 65D (58.6 mg) in dichloromethane (174 μL) was added trifluoroacetic acid (446 μL). The reaction mixture was stirred for 8 hours at ambient temperature. The reaction mixture was concentrated in vacuo and stored in a freezer overnight. To the residue was added to cold aqueous sodium bicarbonate solution (5%) and extracted twice with dichloromethane. The combined organic phases were dried via DryDisk® and concentrated in vacuo. The residue was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.72 (s, 1H), 8.29 (d, 1H), 7.20 (m, 2H), 7.14 (m, 2H), 6.80-6.75 (m, 3H), 6.17 (m, 1H), 5.81 (s, 1H), 4.95-4.85 (m, 3H), 4.45-4.40 (m, 4H), 4.07 (m, 2H), 3.85 (m, 2H), 3.56 (m, 1H), 3.45-3.40 (m, 4H), 2.93 (m, 1H), 2.70-2.25 (m, 11H), 2.18 (s, 3H), 1.97 (s, 6H), 1.74 (m, 1H). MS (ESI) m/z 974.2 (M+H)⁺.

### Example 66

### (7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 66A

### 2-((1r,4r)-4-(2,5,8,11,14-pentaoxahexadecan-16-yloxy)cyclohexyl)-4-(((tert-butyldiphenylsilyl)oxy)methyl)pyrimidine

To a suspension of NaH (60% oil dispersion, 330 mg) in tetrahydrofuran (5 mL), a solution of Example 57E (256 mg) in tetrahydrofuran (4 mL) was added dropwise at room temperature and the resulting suspension was stirred at room temperature for 1 hour. To the mixture, tetra-n-butylammonium iodide (78 mg) and 16-bromo-2,5,8,11,14-pentaoxahexadecane (458 mg) were added. The mixture was stirred two days at room temperature. The mixture was quenched with aqueous ammonium chloride, extracted with ethyl acetate (300 mL), washed with water and brine and dried over sodium sulfate. Filtration and evaporation of the solvent gave the crude product which was loaded on a 40 g column and eluted with 5% methanol in dichloromethane to give the title compound. MS (ESI) m/z 681.3 (M+H)⁺.

### Example 66B

### (2-((1r,4r)-4-(2,5,8,11,14-pentaoxahexadecan-16-yloxy)cyclohexyl)pyrimidin-4-yl)methanol

Example 66B was prepared according to the procedure for Example 57G, substituting Example 66A for Example 57F. MS (ESI) m/z 443.3 (M+H)⁺.

### Example 66C

### tert-butyl (7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylate

Example 66C was prepared according to the procedure for Example 57H, substituting Example 66B for Example 57G. MS (ESI) m/z 1234.5 (M+H)⁺.

### Example 66D

### (7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic

Example 66D was prepared according to the procedure for Example 57I, substituting Example 66C for Example 57H. ¹H NMR (400 MHz, dimethylsulfoxide-d₆) δ ppm 8.70-8.63 (m, 2H), 7.39 (d, 1H), 7.20-7.06 (m, 4H), 6.79 (d, 1H), 6.69 (dd, 1H), 6.15 (dd, 1H), 5.79 (d, 1H), 5.09 (d, 1H), 5.01 (d, 1H), 4.89-4.79 (m, 1H), 4.40 (d, 2H), 3.58-3.44 (m, 18H), 3.39 (dd, 3H), 3.20 (s, 3H), 2.95-2.85 (m, 1H), 2.79-2.56 (m, 3H), 2.42 (s, 3H), 2.36 (s, 4H), 2.16 (s, 3H), 2.08-1.99 (m, 2H), 1.93 (d, 8H), 1.57 (qd, 2H), 1.31-1.17 (m, 2H). MS (ESI) m/z 1179.4 (M+H)⁺.

### Example 67

### (7R,16R)-19,23-dichloro-1-(4-fluorophenyl)-10-[(2-{(1r,4r)-4-[(2,5,8,11,14,17-hexaoxanonadecan-19-yl)oxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 67A

### 2-((1r,4r)-4-(2,5,8,11,14,17-hexaoxanonadecan-19-yloxy)cyclohexyl)-4-(((tert-butyldiphenylsilyl)oxy)methyl)pyrimidine

Example 67A was prepared according to the procedure for Example 66A, substituting 19-bromo-2,5,8,11,14,17-hexaoxanonadecane for 16-bromo-2,5,8,11,14-pentaoxahexadecane. MS (ESI) m/z 725.4 (M+H)⁺.

### Example 67B

### (2-((1r,4r)-4-(2,5,8,11,14,17-hexaoxanonadecan-19-yloxy)cyclohexyl)pyrimidin-4-yl)methanol

Example 67B was prepared according to the procedure for Example 57G, substituting Example 67A for Example 57F. MS (ESI) m/z 487.2 (M+H)⁺.

### Example 67C

### tert-butyl (7R,16R)-19,23-dichloro-1-(4-fluorophenyl)-10-[(2-{(1r,4r)-4-[(2,5,8,11,14,17-hexaoxanonadecan-19-yl)oxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 67C was prepared according to the procedure for Example 57H, substituting Example 67B for Example 57G. MS (ESI) m/z 1277.7 (M+H)⁺.

### Example 67D

### (7R,16R)-19,23-dichloro-1-(4-fluorophenyl)-10-[(2-{(1r,4r)-4-[(2,5,8,11,14,17-hexaoxanonadecan-19-yl)oxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 67D was prepared according to the procedure for Example 57I, substituting Example 67C for Example 57H. ¹H NMR (500 MHz, dimethylsulfoxide-d₆) δ ppm 8.76-8.68 (m, 2H), 7.42 (d, 1H), 7.24-7.16 (m, 2H), 7.19-7.10 (m, 2H), 6.84 (d, 1H), 6.74 (dd, 1H), 6.21 (dd, 1H), 5.81 (d, 1H), 5.13 (d, 1H), 5.05 (d, 1H), 4.87 (p, 1H), 4.44 (d, 2H), 3.64-3.47 (m, 22H), 3.45-3.39 (m, 2H), 3.23 (s, 3H), 2.98-2.90 (m, 1H), 2.82-2.61 (m, 3H), 2.44 (s, 6H), 2.22 (s, 3H), 2.11-2.04 (m, 2H), 1.97 (d, 7H), 1.63 (dd, 1H), 1.58 (dd, 1H), 1.33-1.22 (m, 2H). MS (ESI) m/z 1223.4 (M+H)⁺.

### Example 68

### (7R,16R)-19,23-dichloro-1-(4-fluorophenyl)-20,22-dimethyl-16-[(4-methylpiperazin-1-yl)methyl]-10-({2-[(2S)-2-(2,5,8,11-tetraoxadodecan-1-yl) morpholin-4-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 68A

### (S)(4-(4-(((tert-butyldimethylsilyl)oxy)methyl)pyrimidin-2-yl) morpholin-2-yl)methanol

To a mixture of Example 38A (352 mg) and (S)-morpholin-2-ylmethanol HCl salt (334 mg) in dioxane (5 mL) was added N,N-diisopropylethylamine (0.950 mL). The mixture was stirred at ambient temperature for 5 minutes, heated at 90° C. for 5 hours, diluted with ethyl acetate, washed with water/brine, dried over sodium sulfate, filtered, and concentrated. The residue was purified by flash chromatography on a Teledyne Isco CombiFlash® system, eluting with 0-50% ethyl acetate in heptanes to provide the title compound. MS (APCI) m/z 340.4 (M+H)⁺.

### Example 68B

### (S)-4-(4-(((tert-butyldimethylsilyl)oxy)methyl)pyrimidin-2-yl)-2-(2,5,8,11-tetraoxadodecyl)morpholine

To a mixture of Example 68A (200 mg) and 1-bromo-2-(2-(2-methoxyethoxy)ethoxy) ethane (237 mg) in tetrahydrofuran (2 mL) was added NaH (28 mg). The mixture was heated at 40° C. for 2 hours, diluted with ethyl acetate, washed with water/brine, dried over sodium sulfate, filtered and concentrated. The residue was purified by flash chromatography on a Teledyne Isco CombiFlash® system, eluting with 0-50% methanol in ethyl acetate to provide the title compound. MS (APCI) m/z 486.2 (M+H)⁺.

### Example 68C

### (S)-(2-(2-(2,5,8,11-tetraoxadodecyl) morpholino)pyrimidin-4-yl)methanol

To a mixture of Example 68B (120 mg) in methanol (5 mL) was added 37% concentrated hydrochloric acid (0.113 mL). The mixture was stirred for 15 minutes and was concentrated. The residue was mixed with N,N-diisopropylethylamine (0.1 mL) and methanol (1 mL) and was concentrated. The residue was purified by flash chromatography on a Teledyne Isco CombiFlash® system, eluting with 0-3% methanol in ethyl acetate to provide the title compound. MS (ESI) m/z 372.4 (M+H)⁺.

### Example 68D

### tert-butyl (7R,16R)-19,23-dichloro-1-(4-fluorophenyl)-20,22-dimethyl-16-[(4-methylpiperazin-1-yl)methyl]-10-({2-[(2S)-2-(2,5,8,11-tetraoxadodecan-1-yl) morpholin-4-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 28E by replacing Example 12P and Example 28D with Example 16N and Example 68C, respectively. MS (APCI) m/z 1142.4 (M+H)⁺.

### Example 68E

### (7R,16R)-19,23-dichloro-1-(4-fluorophenyl)-20,22-dimethyl-16-[(4-methylpiperazin-1-yl)methyl]-10-({2-[(2S)-2-(2,5,8,11-tetraoxadodecan-1-yl) morpholin-4-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 28F by replacing Example 28E with Example 68D. ¹H NMR (501 MHz, dimethylsulfoxide-d₆) δ ppm 8.74 (s, 1H), 8.35 (d, 1H), 7.25-7.09 (m, 4H), 6.85-6.69 (m, 3H), 6.22 (dd, 1H), 5.79 (d, 1H), 5.02-4.89 (m, 2H), 4.86 (p, 1H), 4.58-4.48 (m, 1H), 4.48-4.35 (m, 3H), 3.98-3.86 (m, 1H), 3.63-3.45 (m, 17H), 3.41 (dd, 3H), 3.22 (s, 4H), 2.95 (ddd, 3H), 2.79-2.58 (m, 4H), 2.39 (s, 3H), 2.20 (s, 3H), 1.97 (d, 6H). MS (ESI) m/z 1106.5 (M+H)⁺.

### Example 69

### (7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11-tetraoxatridecan-13-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 69A

### 2-((1r,4r)-4-(2,5,8,11-tetraoxatridecan-13-yloxy)cyclohexyl)-4-(((tert-butyldiphenylsilyl)oxy)methyl)pyrimidine

Example 69A was prepared according to the procedure for Example 66A, substituting 13-bromo-2,5,8,11-tetraoxatridecane for 16-bromo-2,5,8,11,14-pentaoxahexadecane. MS (ESI) m/z 637.3 (M+H)⁺.

### Example 69B

### (2-((1r,4r)-4-(2,5,8,11-tetraoxatridecan-13-yloxy)cyclohexyl)pyrimidin-4-yl)methanol

Example 69B was prepared according to the procedure for Example 57G, substituting Example 69A for Example 57F. MS (ESI) m/z 399.4 (M+H)⁺.

### Example 69C

### tert-butyl (7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11-tetraoxatridecan-13-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylate

Example 69C was prepared according to the procedure for Example 57H, substituting Example 69B for Example 57G. MS (ESI) m/z 1191.4 (M+H)⁺.

### Example 69D

### (7R,16R)-19,23-dichloro-1-(4-fluorophenyl)-20,22-dimethyl-16-[(4-methylpiperazin-1-yl)methyl]-10-[(2-{(1r,4r)-4-[(2,5,8,11-tetraoxatridecan-13-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 69D was prepared according to the procedure for Example 57I, substituting Example 69C for Example 57H. ¹H NMR (400 MHz, dimethylsulfoxide-d₆) δ ppm 8.74-8.67 (m, 2H), 7.42 (d, 1H), 7.24-7.09 (m, 4H), 6.83 (d, 1H), 6.73 (dd, 1H), 6.20 (dd, 1H), 5.82 (d, 1H), 5.12 (d, 1H), 5.05 (d, 1H), 4.87 (p, 1H), 4.44 (d, 2H), 3.63-3.48 (m, 14H), 3.43 (dd, 2H), 3.23 (s, 2H), 2.94 (dd, 1H), 2.82-2.60 (m, 3H), 2.45-2.39 (m, 8H), 2.21 (s, 3H), 2.12-2.03 (m, 2H), 1.97 (d, 1H), 1.97 (s, 6H), 1.63 (dd, 1H), 1.57 (dd, 1H), 1.35-1.20 (m, 2H). MS (ESI) m/z 1135.5 (M+H)⁺.

### Example 70

### (7R,16R)-19,23-dichloro-1-(4-fluorophenyl)-10-({2-[(1s,4s)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 70A

### (1s,4s)-4-(4-(((tert-butyldiphenylsilyl)oxy)methyl)pyrimidin-2-yl)cyclohexanol

To a solution of 4-(4-(((tert-butyldiphenylsilyl)oxy)methyl)pyrimidin-2-yl)cyclohexanone (2.2 g) in tetrahydrofuran (20 mL) was added NaBH₄ (0.56 g). The mixture was stirred at room temperature for 3 hours. The mixture was diluted with water (20 mL) and ethyl acetate (300 mL). The organic layer was separated and washed with water and brine and dried over sodium sulfate. Filtration and evaporation of the solvent gave crude product which was loaded on a Redi-Sep Gold 120 g column and eluted with 40% ethyl acetate in heptane to give the title compound. MS (ESI) m/z 447.3 (M+H)⁺.

### Example 70B 4-(((tert-butyldiphenylsilyl)oxy)methyl)-2-((1s,4s)-4-(2-(2-(2-methoxyethoxy)ethoxy)ethoxy)cyclohexyl)pyrimidine

Example 70B was prepared according to the procedure for Example 57F, substituting Example 70A for Example 57E. MS (ESI) m/z 593.5 (M+H)⁺.

### Example 70C

### (2-((1s,4s)-4-(2-(2-(2-methoxyethoxy)ethoxy)ethoxy)cyclohexyl)pyrimidin-4-yl)methanol

Example 70C was prepared according to the procedure for Example 57G, substituting Example 70B for Example 57F. MS (ESI) m/z 355.4 (M+H)⁺.

### Example 70D

### tert-butyl (7R,16R)-19,23-dichloro-1-(4-fluorophenyl)-10-({2-[(1s,4s)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 70D was prepared according to the procedure for Example 57H, substituting Example 70C for Example 57G. MS (ESI) m/z 1147.3 (M+H)⁺.

### Example 70E

### (7R,16R)-19,23-dichloro-1-(4-fluorophenyl)-10-({2-[(1s,4s)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 70E was prepared according to the procedure for Example 57I, substituting Example 70D for Example 57H. ¹H NMR (400 MHz, dimethylsulfoxide-d₆) δ ppm 8.68-8.61 (m, 2H), 7.34 (d, 1H), 7.18-7.03 (m, 4H), 6.78 (d, 1H), 6.67 (dd, 1H), 6.14 (dd, 1H), 5.74 (d, 1H), 5.07 (d, 1H), 4.99 (d, 1H), 4.80 (p, 1H), 4.37 (d, 2H), 3.59-3.40 (m, 12H), 3.34 (dd, 2H), 3.14 (s, 3H), 2.88 (dd, 1H), 2.83-2.74 (m, 1H), 2.68-2.53 (m, 2H), 2.39 (s, 5H), 2.32 (s, 3H), 2.13 (s, 3H), 1.95-1.81 (m, 8H), 1.78 (dt, 2H), 1.64-1.55 (m, 2H), 1.53-1.40 (m, 2H). MS (ESI) m/z 1089.5 (M+H)⁺.

### Example 71

### (7R,16R)-19,23-dichloro-1-(4-fluorophenyl)-20,22-dimethyl-10-[(2-{4-[2-(4-methyl-4-oxo-1, 4λ⁵-azaphosphinan-1-yl)ethoxy]phenyl}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 71A

### (2-(4-(2-chloroethoxy)phenyl)pyrimidin-4-yl)methanol

To a solution of Example 18C (120 mg) in dimethylformamide (2 mL) were added 2-chloroethyl 4-methylbenzenesulfonate (209 mg) and cesium carbonate (290 mg). The mixture was stirred at 50° C. for 2 hours. The mixture was diluted with dichloromethane and washed with water and brine. The organic layer was dried over sodium sulfate, filtered, and concentrated. The residue was purified by silica gel flash chromatography on AnaLogix IntelliFlash²⁸⁰ system eluting with 10-60% ethyl acetate in hexanes to give the title compound. MS (ESI) m/z 265.3 (M+H)⁺.

### Example 71B

### 1-(2-(4-(4-(hydroxymethyl)pyrimidin-2-yl)phenoxy)ethyl)-4-methyl-1,4-azaphosphinane 4-oxide

To a stirring solution of Example 71A (100 mg) in propiononitrile (3 mL) were added 4-methyl-1,4-azaphosphinane 4-oxide hydrochloric acid salt (96 mg), sodium iodide (85 mg) and sodium carbonate (120 mg). The reaction mixture was stirred at 80° C. for 1 day, and cooled and filtered to collect the material. The material were treated with methanol and filtered to remove the inorganic material, and the filtrate was concentrated to give the crude product. The crude product was dissolved in N,N-dimethylformamide and acetonitrile and purified by reverse phase chromatography using a 5-100% gradient of acetonitrile in water (with 0.1% ammonium acetate) over 30 minutes on a Grace Reveleris equipped with a Luna™ column: C18(2), 100 Å, 250×50 mm. The fractions containing the desired compound were combined, frozen and lyophilized to isolate the title compound. MS (ESI) m/z 362.3 (M+H)⁺.

### Example 71C

### tert-butyl (7R,16R)-19,23-dichloro-1-(4-fluorophenyl)-20,22-dimethyl-10-[(2-{4-[2-(4-methyl-4-oxo-1,4λ⁵-azaphosphinan-1-yl)ethoxy]phenyl}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 71B for Example 51D in Example 51E. MS (ESI) m/z 1152.4 (M+H)⁺.

### Example 71D

### (7R,16R)-19,23-dichloro-1-(4-fluorophenyl)-20,22-dimethyl-10-[(2-{4-[2-(4-methyl-4-oxo-1, 4λ⁵-azaphosphinan-1-yl)ethoxy]phenyl}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 71C for Example 51E in Example 51F. ¹H NMR (400 MHz, dimethylsulfoxide-d₆) δ ppm 8.81 (d, 1H), 8.73 (s, 1H), 8.36-8.26 (m, 2H), 7.44 (d, 1H), 7.23-7.09 (m, 4H), 7.08-7.01 (m, 2H), 6.87 (d, 1H), 6.73 (dd, 1H), 6.22 (dd, 1H), 5.83 (d, 1H), 5.19 (q, 2H), 4.86 (p, 1H), 4.43 (d, 2H), 4.14 (t, 2H), 3.77-2.90 (m, 7H), 2.86 (t, 2H), 2.77-2.61 (m, 4H), 2.47-2.30 (m, 5H), 2.17 (s, 3H), 1.97 (s, 3H), 1.96 (s, 3H), 1.88-1.67 (m, 4H), 1.42 (d, 3H). MS (ESI) m/z 1096.6 (M+H)⁺.

### Example 72

### (7R,16R)-19,23-dichloro-10-{[2-(1-{[2-(2-{[(2R)-1,4-dioxan-2-yl]methoxy}ethoxy)ethoxy]methyl}cyclobutyl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 72A

### (2-(2-bromoethoxy)ethoxy) (tert-butyl)diphenylsilane

2-(2-Bromoethoxy) ethan-1-ol (500 mg) was dissolved in dichloromethane (6.0 mL) then imidazole (403 mg) and tert-butyldiphenylchlorosilane (1.0 mL) were added and the resulting mixture was stirred at room temperature for 4 hours. The mixture was then concentrated onto silica gel and purification by flash chromatography on a CombiFlash® Teledyne Isco system using a Teledyne Isco RediSep® Rf gold 80 g silica gel column (eluting 0-20% ethyl acetate/heptane) afforded the title compound. ¹H NMR (500 MHz, dimethylsulfoxide-d₆) δ ppm 7.70-7.62 (m, 4H), 7.51-7.38 (m, 6H), 3.84-3.71 (m, 4H), 3.64-3.52 (m, 4H), 1.00 (s, 9H).

### Example 72B

### 4-(dimethoxymethyl)-2-(1-(10,10-dimethyl-9,9-diphenyl-2,5,8-trioxa-9-silaundecyl)cyclobutyl)pyrimidine

Example 72B was synthesized according to the procedure described for Example 44F, substituting Example 72A for Example 44E. MS (APCI) m/z 565.3 (M+H)⁺.

### Example 72C

### 2-(2-((1-(4-(dimethoxymethyl)pyrimidin-2-yl)cyclobutyl)methoxy)ethoxy)ethanol

To a stirring mixture of Example 72B (350 mg) in 2.2 mL of tetrahydrofuran was added a 1 molar solution of tetrabutyl ammonium fluoride (1.9 mL, in tetrahydrofuran) and the mixture was stirred at room temperature for 30 minutes. The mixture was next concentrated onto silica gel and purification by flash chromatography on a CombiFlash® Teledyne Isco system using a Teledyne Isco RediSep® Rf gold 12 g silica gel column (eluting with solvent A=2:1 ethyl acetate:ethanol, solvent B=heptane; 10-70% A to B) afforded the title compound. MS (APCI) m/z 327.4 (M+H)⁺.

### Example 72D

### (R)-(1,4-dioxan-2-yl)methyl methanesulfonate

A mixture of (S)-(1,4-dioxan-2-yl)methanol (500 mg) with triethylamine (1.7 mL) in 10 mL of dichloromethane was stirred at 0° C. and methanosulfonyl chloride (0.5 mL) was added dropwise. Upon completion of the addition, the cooling bath was removed and the mixture was stirred at room temperature for an hour. The mixture was concentrated onto silica gel and purification by flash chromatography on a CombiFlash® Teledyne Isco system using a Teledyne Isco RediSep® Rf gold 40 g silica gel column (eluting with 30-100% ethyl acetate/heptaneane) afforded the title compound. ¹H NMR (400 MHz, dimethylsulfoxide-d₆) δ ppm 4.24-4.13 (m, 2H), 3.81-3.71 (m, 3H), 3.67-3.56 (m, 2H), 3.51-3.42 (m, 1H), 3.33-3.27 (m, 1H), 3.19 (s, 3H).

### Example 72E

### (R)-2-(1-((2-(2-((1,4-dioxan-2-yl)methoxy)ethoxy)ethoxy)methyl)cyclobutyl)-4-(dimethoxymethyl)pyrimidine

To a stirring solution of Example 72C (155 mg) and Example 72D (186 mg) in acetonitrile (5.0 mL) was added sodium hydride (23 mg) in one portion. The mixture was next stirred at 45° C. for 5 hours. After cooling to ambient temperature, the mixture was quenched with five drops of saturated aqueous ammonium and concentrated onto silica gel. Purification by flash chromatography on a CombiFlash® Teledyne Isco system using a Teledyne Isco RediSep® Rf gold 12 g silica gel column (eluting solvent A=2:1 ethyl acetate:ethanol; solvent B=heptane, 10-100% A to B) afforded the title compound. MS (APCI) m/z 427.3 (M+H)⁺.

### Example 72F

### (R)-2-(1-((2-(2-((1,4-dioxan-2-yl)methoxy)ethoxy)ethoxy)methyl)cyclobutyl)pyrimidine-4-carbaldehyde

Example 72F was synthesized according to the procedure described for Example 29G, substituting Example 72E for Example 29F. MS (APCI) m/z 381.4 (M+H)⁺.

### Example 72G

### (R)-(2-(1-((2-(2-((1,4-dioxan-2-yl)methoxy)ethoxy)ethoxy)methyl)cyclobutyl)pyrimidin-4-yl)methanol

Example 72G was synthesized according to the procedure described for Example 29H, substituting Example 72F for Example 29G. MS (APCI) m/z 383.4 (M+H)⁺.

### Example 72H

### tert-butyl (7R,16R)-19,23-dichloro-10-{[2-(1-{[2-(2-{[(2R)-1,4-dioxan-2-yl]methoxy}ethoxy)ethoxy]methyl}cyclobutyl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 72H was synthesized according to the procedure described for Example 291, substituting Example 72G for Example 29H. MS (APCI) m/z 1175.4 (M+H)⁺.

### Example 72I

### (7R,16R)-19,23-dichloro-10-{[2-(1-{[2-(2-{[(2R)-1,4-dioxan-2-yl]methoxy}ethoxy)ethoxy]methyl}cyclobutyl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 721 was synthesized according to the procedure described for Example 29J, substituting Example 72H for Example 291. ¹H NMR (500 MHz, dimethylsulfoxide-d₆) δ ppm 8.75 (d, Hz, 1H), 8.73 (s, 1H), 7.42 (d, 1H), 7.29-7.08 (m, 4H), 6.88 (d, 1H), 6.75 (dd, 1H), 6.24 (dd, 1H), 5.81 (d, 1H), 5.11 (q, 2H), 4.88 (p, 1H), 4.44 (d, 2H), 3.86 (s, 2H), 3.69-3.17 (m, 17H), 3.01-2.91 (m, 1H), 2.78-2.61 (m, 2H), 2.61-2.38 (m, 11H), 2.24 (s, 3H), 2.21-2.09 (m, 2H), 2.05-1.90 (m, 7H), 1.87-1.72 (m, 1H). MS (APCI) m/z 1119.5 (M+H)⁺.

### Example 73

### (7R,16R)-19,23-dichloro-10-{[2-(1,1-dioxo-1λ⁶-thiolan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 73A

### (2-(1,1-dioxidotetrahydrothiophen-3-yl)pyrimidin-4-yl)methyl methanesulfonate

3-(4-(Hydroxymethyl)pyrimidin-2-yl)tetrahydrothiophene 1,1-dioxide (50 mg) was dissolved in dichloromethane (3 mL) under a nitrogen atmosphere and cooled to 0° C. with ice water. Triethylamine (92 μL) and methanesulfonyl chloride (5 μL) were added and the reaction mixture was stirred under cooling for 5 hours. Brine was added to the reaction mixture and the aqueous layer was extracted with dichloromethane. The combined organic extract were dried via DryDisk®, filtered and concentrated in vacuo. The crude product was used without any further purification in the next step. MS (APCI) m/z 307.0 (M+H)⁺.

### Example 73B

### tert-butyl (7R,16R)-19,23-dichloro-10-{[2-(1,1-dioxo-1λ⁶-thiolan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A 4 mL vial, equipped with stir bar, was charged with Example 16N (50 mg) and Example 73A (34 mg). N,N-Dimethylformamide (206 μL) and cesium carbonate (60.4 mg) were added. The reaction mixture was stirred overnight at ambient temperature. The reaction mixture was added to cold aqueous sodium bicarbonate solution (5%). The precipitate was filtered off after 5 minutes and washed twice with cold water. The precipitate was dried in vacuo overnight at 30° C. MS (ESI) m/z 1019.3 (M+H)⁺.

### Example 73C

### (7R,16R)-19,23-dichloro-10-{[2-(1,1-dioxo-1λ⁶-thiolan-3-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

To a solution of Example 73B (59.1 mg) in dichloromethane (386 μL) was added trifluoroacetic acid (446 μL). The reaction mixture was stirred for 20 hours at ambient temperature. The reaction mixture was then concentrated in vacuo. To the residue was added to cold aqueous sodium bicarbonate solution (5%) and the mixture was extracted twice with dichloromethane. The combined organic phases were dried via DryDisk® and concentrated in vacuo. The residue was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.81 (d, 1H), 8.74 (s, 1H), 7.55 (d, 1H), 7.20 (m, 2H), 7.14 (m, 2H), 6.88 (d, 1H), 6.76 (m, 1H), 6.20 (m, 1H), 5.80 (s, 1H), 5.17 (d, 1H), 5.14 (d, 1H), 4.88 (m, 1H), 4.44 (m, 2H), 3.94 (m, 1H), 3.59 (m, 2H), 3.46 (m, 1H), 3.24 (m, 2H), 2.95 (m, 1H), 2.68 (m, 2H), 2.60-2.25 (m, 10H), 2.19 (s, 3H), 1.99 (s, 3H), 1.97 (s, 3H). MS (ESI) m/z 963.2 (M+H)⁺.

### Example 74

### (7R,16R)-19,23-dichloro-1-(4-fluorophenyl)-10-[(2-{1-[(2,5,8,11,14,17-hexaoxanonadecan-19-yl)oxy]cyclopentyl}pyrimidin-4-yl)methoxy]-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 74A

### 1-(2,5,8,11,14,17-hexaoxanonadecan-19-yloxy)cyclopentanecarbonitrile

Zinc chloride (1.226 g) was heated at 120° C. under vacuum overnight, and cooled. 2,5,8,11,14,17-Hexaoxanonadecan-19-ol (4.00 g) was added, 1-hydroxycyclopentanecarbonitrile (1 g) was added, and the reaction was heated to 60° C. overnight. The material was taken up in ethyl acetate and a minimal amount of water, the layers were separated, and the aqueous layer was extracted five times with ether. The organic layers were combined, dried over sodium sulfate, filtered and concentrated. The crude product was purified by reverse phase chromatography using a 10-65% gradient of acetonitrile in water (with 0.1% ammonium acetate) over 35 minutes on a Grace Reveleris equipped with a Luna™ column: C18(2), 100 Å, 250×50 mm to isolate the title compound. ¹H NMR (400 MHz, dimethylsulfoxide-d₆) δ ppm 3.67 (m, 16H), 3.62 (m, 2H), 3.55 (m, 2H), 3.50 (m, 2H), 3.42 (m, 2H), 3.24 (s, 3H), 2.04 (m, 4H), 1.70 (m, 4H). MS (ESI) m/z 407.1 (M+NH₄)⁺.

### Example 74B 1-(2,5,8,11,14,17-hexaoxanonadecan-19-yloxy)cyclopentanecarboximidamide acetate

Hydroxylamine hydrochloride (250 mg) and sodium carbonate (381 mg) were added to Example 74A (700 mg) in 8 mL ethanol and 0.15 mL water, and the reaction was heated to 80° C. overnight. The reaction was then cooled, filtered and concentrated. The residue was taken up in 4 mL acetic acid and 2 mL acetic anhydride and stirred overnight. The solution was concentrated, then concentrated twice from heptanes, and subjected to high vacuum overnight. The material was then taken up in methanol (7.4 mL) and added to 5% wet Pd/C (0.25 g) in a 20 mL Barnstead Hast C reactor, and purged with argon. The mixture was stirred at 1200 rpm under 50 psi of hydrogen at 25° C. for 2.6 hours. The mixture was filtered through a filter funnel with a polyethylene frit packed with diatomaceous earth and concentrated to give the title compound as an acetate salt. ¹H NMR (400 MHz, dimethylsulfoxide-d₆) δ ppm 3.56 (m, 4H), 3.52 (m, 16H), 3.42 (m, 4H), 3.24 (s, 3H), 2.00 (m, 2H), 1.89 (m, 2H), 1.77 (s, 3H), 1.73 (m, 2H), 1.70 (m, 2H).

### Example 74C

### 2-(1-(2,5,8,11,14,17-hexaoxanonadecan-19-yloxy)cyclopentyl)-4-(dimethoxymethyl)pyrimidine

Example 74B (900 mg) and (E)-4-(dimethylamino)-1,1-dimethoxybut-3-en-2-one (278 mg) were stirred in 12 mL dry methanol. To the mixture was added sodium methoxide (347 mg, 25% wt solution in methanol), and the reaction was stirred at 75° C. for 6 hours. The reaction was cooled and partitioned between 200 mL ethyl acetate and 20 mL pH 7 buffer, and the organic layer was concentrated. The crude material was purified by reverse phase chromatography using a 10-80% gradient of acetonitrile in water (with 0.1% ammonium acetate) over 35 minutes on a Grace Reveleris equipped with a Luna™ column: C18(2), 100 Å, 250×50 mm to isolate the cyclized acetal. The material was taken up in 20 mL 2M aqueous HCl at 60° C. for 1 hour. The solution was cooled to 0° C. Concentrated aqueous NaOH solution was slowly added portionwise. The pH was adjusted to 8 using 10% potassium carbonate solution, sodium borohydride (117 mg) was added portionwise keeping the temperature under 5° C., and the mixture was stirred for 10 minutes at 0° C. The reaction mixture was added to pH 7 buffer and extracted three times with ethyl acetate. The combined extracts were dried over sodium sulfate, filtered and concentrated to give the title compound. ¹H NMR (400 MHz, dimethylsulfoxide-d₆) δ ppm 8.78 (d, 1H), 7.44 (d, 1H), 5.60 (t, 1H), 4.55 (d, 2H), 3.50 (m, 14H), 3.47 (m, 4H), 3.42 (m, 4H), 3.27 (m, 2H), 3.24 (s, 3H), 2.07 (m, 4H), 1.75 (m, 2H), 1.65 (m, 2H). MS (ESI) m/z 473.2 (M+H)⁺.

### Example 74D

### (7R,16R)-19,23-dichloro-1-(4-fluorophenyl)-10-[(2-{1-[(2,5,8,11,14,17-hexaoxanonadecan-19-yl)oxy]cyclopentyl}pyrimidin-4-yl)methoxy]-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was prepared by substituting Example 74C for Example 38D in Example 38E. ¹H NMR (400 MHz, dimethylsulfoxide-d₆) δ ppm 8.79 (d, 1H), 8.71 (s, 1H), 7.52 (dd, 1H), 7.20 (m, 2H), 7.13 (m, 2H), 6.84 (dd, 1H), 6.71 (dd, 1H), 6.17 (d, 1H), 5.84 (d, 1H), 5.12 (dd, 2H), 4.91 (m, 1H), 4.44 (d, 2H), 3.56 (m, 2H), 3.48 (m, 20H), 3.26 (m, 4H), 3.22 (s, 3H), 2.94 (m, 1H), 2.67 (m, 2H), 2.46 (m, 5H), 2.36 (m, 2H), 2.17 (s, 3H), 2.09 (m, 4H), 1.98 (s, 3H), 1.95 (s, 3H), 1.76 (m, 2H), 1.65 (m, 2H). MS (ESI) m/z 604.3 ((M+H)/2)⁺.

### Example 75

### (7R,16R)-19,23-dichloro-10-{[2-(1,1-dioxo-1λ⁶-thiomorpholin-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 75A

### methyl 2-(1,1-dioxidothiomorpholino)pyrimidine-4-carboxylate

Methyl 2-chloropyrimidine-4-carboxylate (300 mg) and thiomorpholine 1,1-dioxide (306 mg) were dissolved in dioxane (10 mL) under argon atmosphere. Triethylamine (0.97 mL) was added and the reaction mixture was degassed with argon for 15 minutes. The reaction mixture was stirred at 80° C. for 12 hours. Dioxane was evaporated and the residue diluted with dichloromethane. The organic phase was washed with brine and aqueous sodium bicarbonate solution. The aqueous layer was extracted with dichloromethane (2 times). The combined organic layers were dried over magnesium sulfate, filtrated, and concentrated. Purification was performed on a silica gel column (12 g, 0-2% methanol in dichloromethane). The desired fractions were combined and the solvents were removed under reduced pressure to provide the title compound. MS (ESI) m/z 272.1 (M+H)⁺.

### Example 75B

### 4-(4-(hydroxymethyl)pyrimidin-2-yl)thiomorpholine 1,1-dioxide

Example 75A (105 mg) was dissolved in methanol (3.0 mL) under nitrogen, cooled to 0° C. with an ice-bath, and sodium borohydride (45 mg) was added. The reaction mixture was stirred at 0° C. for 10 minutes, and the reaction mixture was allowed to warm to room temperature, and stirred overnight. Additional sodium borohydride (30 mg) was added and the reaction mixture was stirred at room temperature for additional 2 hours. The mixture was concentrated. Aqueous saturated sodium bicarbonate solution was added (until pH 9). The aqueous phase was extracted with dichloromethane (3 times). The combined organic layers were dried over magnesium sulfate, filtrated and concentrated to yield the title compound, which was used in the next step without further purification. MS (APCI) m/z 244.2 (M+H)⁺.

### Example 75C

### (2-(1,1-dioxidothiomorpholino)pyrimidin-4-yl)methyl methanesulfonate

Example 75B (88 mg) was dissolved in dichloromethane under nitrogen atmosphere and cooled with an ice-bath to 0° C. Triethylamine (0.15 mL) and methanesulfonyl chloride (34 μL) were added, and the reaction mixture was stirred at 0° C. for 150 minutes. The reaction mixture was diluted with brine, and the aqueous layer was extracted with dichloromethane (2 times). The combined organic extracts were dried over magnesium sulfate, filtrated, and concentrated to provide the crude title compound. MS (APCI) m/z 322.1 (M+H)⁺.

### Example 75D

### tert-butyl (7R,16R)-19,23-dichloro-10-{[2-(1,1-dioxo-1λ⁶-thiomorpholin-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 75C (34 mg), Example 16N (40 mg), and cesium carbonate (53 mg) were combined under nitrogen atmosphere and N,N-dimethylformamide (0.2 mL) was added. The reaction mixture was stirred overnight at room temperature. A 1:1 mixture of water and aqueous saturated sodium bicarbonate solution (2.5 mL) was added to the reaction mixture. The resulting suspension was stirred at room temperature vigorously for 20 minutes. The suspension was filtered and the residue was washed with water (1 mL) and dried over sodium sulfate, filtered, and concentrated to yield the crude title compound. MS (APCI) m/z 1034.3 (M+H)⁺.

### Example 75E

### (7R,16R)-19,23-dichloro-10-{[2-(1,1-dioxo-1λ⁶-thiomorpholin-4-yl)pyrimidin-4-yl]methoxy}-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 75D (49 mg) was dissolved in dichloromethane (360 μL) under nitrogen atmosphere. Trifluoroacetic acid (361 μL) was added and the mixture was stirred at room temperature for 6 hours. The reaction mixture was diluted with dichloromethane and concentrated at room temperature. The obtained residue was again dissolved in dichloromethane and washed with a 1:1 mixture of water and aqueous saturated sodium bicarbonate solution (6 mL). The aqueous layer was extracted with dichloromethane twice. The combined organic layer was dried over magnesium sulfate, filtrated and concentrated. The crude material was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.73 (s, 1H), 8.42 (d, 1H), 7.22-7.19 (m, 2H), 7.15-7.13 (m, 2H), 6.87 (d, 1H), 6.82 (d, 1H), 6.73 (dd, 1H), 6.18 (m, 1H), 5.81 (m, 1H), 5.02 (d, 1H), 4.94 (d, 1H), 4.89 (m, 1H), 4.46-4.41 (m, 2H), 4.21 (m, 4H), 3.75 (dd, 1H), 3.15 (t, 4H), 2.94 (dd, 1H), 2.68 (qd, 2H), 2.54-2.31 (m, 8H), 2.18 (s, 3H), 1.97 (s, 6H). MS (APCI) m/z 978.2 (M+H)⁺.

### Example 76

### (7R,16R)-19,23-dichloro-1-(4-fluorophenyl)-10-({2-[1-(2,5,8,11,14,17-hexaoxaoctadecan-1-yl)cyclopentyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 76A

### 1-(((tert-butyldimethylsilyl)oxy)methyl)cyclopentanecarbonitrile

Example 76A was synthesized according to the procedure described for Example 44A, substituting 1-(hydroxymethyl)cyclopentanecarbonitrile for 1-(hydroxymethyl)cyclobutanecarbonitrile. MS (DCI) m/z 257.1 (M+H+NH3)⁺.

### Example 76B

### 1-(((tert-butyldimethylsilyl)oxy)methyl)cyclopentanecarboximidamide

Example 76B was synthesized according to the procedure described for Example 44B, substituting Example 76A for Example 44A. MS (DCI) m/z 257.1 (M+H)⁺.

### Example 76C

### 2-(1-(((tert-butyldimethylsilyl)oxy)methyl)cyclopentyl)-4-(dimethoxymethyl)pyrimidine

Example 76C was synthesized according to the procedure described for Example 44C, substituting Example 76B for Example 44B. MS (DCI) m/z 367.2 (M+H)⁺.

### Example 76D

### (1-(4-(dimethoxymethyl)pyrimidin-2-yl)cyclopentyl)methanol

Example 76D was synthesized according to the procedure described for Example 44D, substituting Example 76C for Example 44C. MS (DCI) m/z 253.1 (M+H)⁺.

### Example 76E

### 2-(1-(2,5,8,11,14,17,20,23,26,29,32,35,38-tridecaoxanonatriacontyl)cyclobutyl)-4-(dimethoxymethyl)pyrimidine

Example 76E was synthesized according to the procedure described for Example 44F, substituting 16-bromo-2,5,8,11,14-pentaoxahexadecane for Example 44E and substituting Example 76D for Example 44D. MS (APCI) m/z 487.2 (M+H)⁺.

### Example 76F

### 2-(1-(2,5,8,11,14,17-hexaoxaoctadecyl)cyclopentyl)pyrimidine-4-carbaldehyde

Example 76F was synthesized according to the procedure described for Example 29G, substituting Example 76E for Example 29F. MS (APCI) m/z 441.4 (M+H)⁺.

### Example 76G

### (2-(1-(2,5,8,11,14,17-hexaoxaoctadecyl)cyclopentyl)pyrimidin-4-yl)methanol

Example 76G was synthesized according to the procedure described for Example 29H, substituting Example 76F for Example 29G. MS (APCI) m/z 443.4 (M+H)⁺.

### Example 76H

### tert-butyl (7R,16R)-19,23-dichloro-1-(4-fluorophenyl)-10-({2-[1-(2,5,8,11,14,17-hexaoxaoctadecan-1-yl)cyclopentyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylare

Example 76H was synthesized according to the procedure described for Example 291, substituting Example 76G for Example 29H. ¹H NMR (500 MHz, dimethylsulfoxide-d₆) δ ppm 8.77 (d, 1H), 8.74 (s, 1H), 7.41 (d, 1H), 7.29-7.12 (m, 4H), 6.93 (d, 1H), 6.82 (dd, 1H), 6.04 (dd, 1H), 5.68 (d, 1H), 5.23-4.98 (m, 2H), 4.77 (d, 1H), 4.57-4.33 (m, 2H), 3.75 (s, 2H), 3.56-3.38 (m, 20H), 3.22 (s, 3H), 2.88 (d, Hz, 1H), 2.74-2.61 (m, 2H), 2.49-2.25 (m, 12H), 2.25-2.06 (m, 8H), 1.90 (s, 3H), 1.83-1.71 (m, 1H), 1.71-1.51 (m, 2H), 1.06 (s, 9H).

### Example 761

### (7R,16R)-19,23-dichloro-1-(4-fluorophenyl)-10-({2-[1-(2,5,8,11,14,17-hexaoxaoctadecan-1-yl)cyclopentyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 76I was synthesized according to the procedure described for Example 29J, substituting Example 76H for Example 291. ¹H NMR (500 MHz, dimethylsulfoxide-d₆) δ ppm 8.77-8.70 (m, 2H), 7.40 (d, 1H), 7.24-7.09 (m, 4H), 6.87 (d, 1H), 6.74 (dd, 1H), 6.24 (dd, 1H), 5.81 (d, 1H), 5.10 (q, Hz, 2H), 4.88 (p, 1H), 4.52-4.35 (m, 2H), 3.74 (s, 2H), 3.61 (dd, 1H), 3.51-3.44 (m, 10H), 3.44-3.38 (m, 9H), 3.22 (s, 3H), 3.01-2.89 (m, 1H), 2.76-2.61 (m, 2H), 2.49-2.36 (m, 10H), 2.28-2.14 (m, 5H), 1.97 (s, 6H), 1.82-1.70 (m, 2H), 1.70-1.50 (m, 3H). MS (APCI) m/z 1177.5 (M+H)⁺.

### Example 77

### (7R,16R)-19,23-dichloro-10-[(2-{1-[(2-{[(2R)-1,4-dioxan-2-yl]methoxy}ethoxy)methyl]cyclobutyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 77A

### 2-(1-((2-((tert-butyldimethylsilyl)oxy)ethoxy)methyl)cyclobutyl)-4-(dimethoxymethyl)pyrimidine

Example 77A was synthesized according to the procedure described for Example 44F, substituting (2-bromoethoxy) (tert-butyl)dimethylsilane for Example 44E. MS (APCI) m/z 397.4 (M+H)⁺.

### Example 77B

### 2-((1-(4-(dimethoxymethyl)pyrimidin-2-yl)cyclobutyl)methoxy)ethanol

Example 77B was synthesized according to the procedure described for Example 72C, substituting Example 77A for Example 72B. MS (APCI) m/z 283.1 (M+H)⁺.

### Example 77C

### (R)-2-(1-((2-((1,4-dioxan-2-yl)methoxy)ethoxy)methyl)cyclobutyl)-4-(dimethoxymethyl)pyrimidine

Example 77C was synthesized according to the procedure described for Example 72E, substituting Example 77B for Example 72C. MS (APCI) m/z 383.3 (M+H)⁺.

### Example 77D

### (R)-2-(1-((2-((1,4-dioxan-2-yl)methoxy)ethoxy)methyl)cyclobutyl)pyrimidine-4-carbaldehyde

Example 77D was synthesized according to the procedure described for Example 29G, substituting Example 77C for Example 29F. MS (APCI) m/z 337.3 (M+H)⁺.

### Example 77E

### (R)-(2-(1-((2-((1,4-dioxan-2-yl)methoxy)ethoxy)methyl)cyclobutyl)pyrimidin-4-yl)methanol

Example 77E was synthesized according to the procedure described for Example 29H, substituting Example 77D for Example 29G. MS (APCI) m/z 339.4 (M+H)⁺.

### Example 77F

### tert-butyl (7R,16R)-19,23-dichloro-10-[(2-{1-[(2-{[(2R)-1,4-dioxan-2-yl]methoxy}ethoxy)methyl]cyclobutyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 77F was synthesized according to the procedure described for Example 291, substituting Example 77E for Example 29H. MS (APCI) m/z 1131.3 (M+H)⁺.

### Example 77G

### (7R,16R)-19,23-dichloro-10-[(2-{1-[(2-{[(2R)-1,4-dioxan-2-yl]methoxy}ethoxy)methyl]cyclobutyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 77G was synthesized according to the procedure described for Example 29J, substituting Example 77F for Example 291. ¹H NMR (500 MHz, dimethylsulfoxide-d₆) δ ppm 8.75 (d, 1H), 8.73 (s, 1H), 7.43 (d, 1H), 7.24-7.09 (m, 4H), 6.88 (d, 1H), 6.75 (dd, 1H), 6.24 (dd, 1H), 5.82 (d, 1H), 5.20-5.03 (m, 2H), 4.88 (p, 1H), 4.52-4.37 (m, 2H), 3.85 (s, 2H), 3.66-3.45 (m, 7H), 3.44-3.35 (m, 3H), 3.30 (dd, 1H), 3.23 (dd, 1H), 3.16 (dd, 1H), 3.00-2.91 (m, 1H), 2.76-2.61 (m, 2H), 2.49-2.38 (m, 11H), 2.24 (s, 3H), 2.20-2.09 (m, 2H), 2.03-1.91 (m, 7H), 1.86-1.70 (m, 1H). MS (APCI) m/z 1073.3 (M+H)⁺.

### Example 78

### (7R,16R)-19-chloro-10-{[2-(3,3-difluoro-1-oxa-8-azaspiro[4.5]decan-8-yl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 78A (2-(3,3-difluoro-1-oxa-8-azaspiro[4.5]decan-8-yl)pyrimidin-4-yl)methanol

A solution of 3,3-difluoro-1-oxa-8-azaspiro[4.5]decane, hydrochloric acid salt (270 mg), (2-chloropyrimidin-4-yl)methanol (150 mg) and N,N-diisopropylethylamine (910 μL) in acetonitrile (2.6 mL) was heated to 80° C. for 7 hours and stirred overnight at room temperature. The reaction was diluted with water and extracted with ethyl acetate three times. The combined organic layers were dried over anhydrous sodium sulfate, filtered and concentrated. The residue was purified by normal phase MPLC on a Teledyne Isco CombiFlash® Rf+12 g gold silica gel column eluting with 5-70% ethyl acetate in dichloromethane. The desired fractions were concentrated and purified by normal phase MPLC on a Teledyne Isco CombiFlash® Rf+24 g gold silica gel column eluting with 0-40% ethyl acetate in dichloromethane to give the title compound. ¹H NMR (400 MHz, dimethylsulfoxide-d₆) δ ppm 8.32 (d, 1H), 6.69 (d, 1H), 5.44-5.34 (m, 1H), 4.34 (d, 2H), 4.09-3.90 (m, 4H), 3.67-3.53 (m, 2H), 2.43-2.28 (m, 2H), 1.78-1.58 (m, 4H).

### Example 78B

### tert-butyl (7R,16R,21S)-19-chloro-10-{[2-(3,3-difluoro-1-oxa-8-azaspiro[4.5]decan-8-yl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 12P (40 mg), Example 78A (45 mg) and triphenylphosphine (41 mg) in toluene (525 μL) at room temperature was added di-tert-butyl azodicarboxylate (36 mg), and the reaction was allowed to stir overnight. The reaction mixture was concentrated, and the residue was purified by normal phase MPLC on a Teledyne Isco CombiFlash® Rf+12 g gold silica gel column eluting with 0.5-7.5% methanol in dichloromethane to give the title compound.

### Example 78C

### (7R,16R,21S)-19-chloro-10-{[2-(3,3-difluoro-1-oxa-8-azaspiro[4.5]decan-8-yl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 78B (42 mg) in dichloromethane (200 μL) was added trifluoroacetic acid (200 μL), and the reaction was allowed to stir for 4 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-85% over 30 minutes with acetonitrile in water containing 0.01% trifluoroacetic acid) to give the title compound. ¹H NMR (400 MHz, dimethylsulfoxide-d₆) δ ppm 8.74 (s, 1H), 8.34 (d, 1H), 7.26-7.10 (m, 6H), 6.96 (d, 1H), 6.88-6.77 (m, 2H), 6.71 (d, 1H), 6.18-6.09 (m, 1H), 5.70-5.63 (m, 1H), 5.03-4.85 (m, 2H), 4.67-4.57 (m, 1H), 4.51-4.42 (m, 1H), 4.41-4.29 (m, 1H), 4.13-3.91 (m, 4H), 3.87-3.74 (m, 1H), 3.70-3.56 (m, 2H), 3.39 (br s, 2H), 2.93-2.75 (m, 6H), 2.45-2.31 (m, 2H), 2.21 (s, 3H), 1.82-1.59 (m, 4H). MS (ESI) m/z 970.0 (M−H)⁻.

### Example 79

### (7R,16R)-19,23-dichloro-1-(4-fluorophenyl)-10-{[2-(6-methoxy-2-azaspiro[3,3]heptan-2-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 79A

### (2-(6-methoxy-2-azaspiro[3.3]heptan-2-yl)pyrimidin-4-yl)methanol

A mixture of (2-chloropyrimidin-4-yl)methanol (220 mg), 6-methoxy-2-azaspiro[3.3]heptane (HCl salt, 300 mg) and triethylamine (616 mg) in dioxane (2.5 mL) was heated in a Q-tube overnight at 80° C. Excess water was added, followed by extraction with dichloromethane, washing of combined organic layers with water and drying over magnesium sulfate, filtration and concentration. The crude title compound obtained was used without further purification.

### Example 79B

### tert-butyl (7R,16R)-19,23-dichloro-1-(4-fluorophenyl)-10-{[2-(6-methoxy-2-azaspiro[3.3]heptan-2-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A microwave vial was charged with Example 16N (20.0 mg), Example 79A (11.6 mg), N,N,N′,N′-tetramethylazodicarboxamide (17.0 mg) and triphenylphosphine (25.9 mg). After degassing, a mixture of degassed toluene (0.5 mL) and tetrahydrofuran (0.5 mL) was added and the reaction mixture was heated in the microwave for 4.5 hours at 50° C. Water was added (20 mL), followed by extraction with ethyl acetate. The combined organic layers were washed with brine, dried over magnesium sulfate, filtered and concentrated. The crude product was purified by chromatography on silica gel using a CombiFlash® system (4 g RediSep® Gold column, eluting with 0-30% dichloromethane/methanol) providing the title compound. MS (APCI) m/z 1026.3 (M+H)⁺.

### Example 79C

### (7R,16R)-19,23-dichloro-1-(4-fluorophenyl)-10-{[2-(6-methoxy-2-azaspiro[3.3]heptan-2-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Trifluoroacetic acid (0.19 mL) was added to Example 79B (25 mg) in dichloromethane (2.5 mL). The reaction mixture was stirred overnight at room temperature. Removal of the solvent, followed by purification by HPLC (Waters XBridge C8 19×150 mm 5 μm column, gradient 5-100% acetonitrile+0.2% ammonium hydroxide in water+0.2% ammonium hydroxide) provided the title compound. ¹H NMR (600 MHz, dimethylsulfoxide-d₆) δ ppm 8.72 (s, 1H), 8.29 (d, 1H), 7.21-7.17 (m, 2H), 7.13 (m, 2H), 6.80-6.70 (m, 3H), 6.16 (s, 1H), 5.81 (s, 1H), 4.95 (d, 1H), 4.88 (m, 2H), 4.4 (m, 2H), 4.02 (s, 2H), 3.96 (s, 2H), 3.77 (m, 1H), 3.54 (m, 1H), 3.12 (m, 4H), 2.95-2.90 (m, 1H), 2.71-2.63 (m, 2H), 2.48-2.25 (m, 9H), 2.17 (s, 3H), 2.04 (m, 2H), 1.97 (m, 6H). MS (APCI) m/z 970.3 (M+H)⁺.

### Example 80

### (7R,16R)-19,23-dichloro-10-({2-[1-(2-{[(2R)-1,4-dioxan-2-yl]methoxy}ethoxy)-2-methylpropan-2-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 80A

### 3-((tert-butyldimethylsilyl)oxy)-2,2-dimethylpropanenitrile

Example 80A was synthesized according to the procedure described for Example 44A, substituting 3-hydroxy-2,2-dimethylpropanenitrile for 1-(hydroxymethyl)cyclobutanecarbonitrile. MS (APCI) m/z 214.1 (M+H+NH3)⁺.

### Example 80B

### 3-((tert-butyldimethylsilyl)oxy)-2,2-dimethylpropanimidamide

Example 80B was synthesized according to the procedure described for Example 44B, substituting Example 80A for Example 44A. MS (APCI) m/z 231.1 (M+H)⁺.

### Example 80C

### 2-(1-((tert-butyldimethylsilyl)oxy)-2-methylpropan-2-yl)-4-(dimethoxymethyl)pyrimidine

Example 80C was synthesized according to the procedure described for Example 44C, substituting Example 80B for Example 44B. MS (APCI) m/z 341.4 (M+H)⁺.

### Example 80D

### 2-(4-(dimethoxymethyl)pyrimidin-2-yl)-2-methylpropan-1-ol

Example 80D was synthesized according to the procedure described for Example 44D, substituting Example 80C for Example 44C. MS (APCI) m/z 227.4 (M+H)⁺.

### Example 80E

### 2-(1-(2,5,8,11,14,17,20,23,26,29,32,35,38-tridecaoxanonatriacontyl)cyclobutyl)-4-(dimethoxymethyl)pyrimidine

Example 80E was synthesized according to the procedure described for Example 44F, substituting (2-bromoethoxy) (tert-butyl)dimethylsilane for Example 44E and Example 80D for Example 44D. MS (APCI) m/z 385.4 (M+H)⁺.

### Example 80F

### 2-(2-(4-(dimethoxymethyl)pyrimidin-2-yl)-2-methylpropoxy)ethanol

Example 80F was synthesized according to the procedure described for Example 72C, substituting Example 80E for Example 72B. MS (APCI) m/z 271.3 (M+H)⁺.

### Example 80G

### (R)-2-(1-(2-((1,4-dioxan-2-yl)methoxy)ethoxy)-2-methylpropan-2-yl)-4-(dimethoxymethyl)pyrimidine

Example 80G was synthesized according to the procedure described for Example 72E, substituting Example 80F for Example 72C. MS (APCI) m/z 371.4 (M+H)⁺.

### Example 80H

### (R)-2-(1-(2-((1,4-dioxan-2-yl)methoxy)ethoxy)-2-methylpropan-2-yl)pyrimidine-4-carbaldehyde

Example 80H was synthesized according to the procedure described for Example 29G, substituting Example 80G for Example 29F. MS (APCI) m/z 325.4 (M+H)⁺.

### Example 80I

### (R)-(2-(1-(2-((1,4-dioxan-2-yl)methoxy)ethoxy)-2-methylpropan-2-yl)pyrimidin-4-yl)methanol

Example 80I was synthesized according to the procedure described for Example 29H, substituting Example 80H for Example 29G. MS (APCI) m/z 327.4 (M+H)⁺.

### Example 80J

### tert-butyl (7R,16R)-19,23-dichloro-10-({2-[1-(2-{[(2R)-1,4-dioxan-2-yl]methoxy}ethoxy)-2-methylpropan-2-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 80J was synthesized according to the procedure described for Example 29I, substituting Example 801 for Example 29H. MS (APCI) m/z 1119.5 (M+H)⁺.

### Example 80K

### (7R,16R)-19,23-dichloro-10-({2-[1-(2-{[(2R)-1,4-dioxan-2-yl]methoxy}ethoxy)-2-methylpropan-2-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 80K was synthesized according to the procedure described for Example 29J, substituting Example 80J for Example 291. ¹H NMR (500 MHz, dimethylsulfoxide-d₆) δ ppm 8.79-8.67 (m, 2H), 7.43 (d, 1H), 7.22-7.17 (m, 2H), 7.17-7.11 (m, 2H), 6.87 (d, 1H), 6.75 (dd, 1H), 6.24 (dd, 1H), 5.80 (d, 1H), 5.18-5.00 (m, 2H), 4.95-4.84 (m, 1H), 4.51-4.33 (m, 2H), 3.68 (s, 2H), 3.67-3.47 (m, 7H), 3.44-3.40 (m, 4H), 3.30 (dd, 1H), 3.23 (dd, 1H), 3.17 (dd, 1H), 2.95 (dd, 1H), 2.76-2.62 (m, 2H), 2.49-2.34 (m, 8H), 2.22 (s, 3H), 1.97 (d, 6H), 1.30 (s, 6H). MS (APCI) m/z 1063.0 (M+H)⁺.

### Example 81

### (7R,16R)-19,23-dichloro-10-[(2-{1-[(2-{[(2R)-1,4-dioxan-2-yl]methoxy}ethoxy)methyl]cyclopentyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 81A

### 2-(1-((2-((tert-butyldimethylsilyl)oxy)ethoxy)methyl)cyclopentyl)-4-(dimethoxymethyl)pyrimidine

Example 81A was synthesized according to the procedure described for Example 44F, substituting (2-bromoethoxy) (tert-butyl)dimethylsilane for Example 44E and Example 76D for Example 44D. MS (APCI) m/z 411.4 (M+H)⁺.

### Example 81B

### 2-((1-(4-(dimethoxymethyl)pyrimidin-2-yl)cyclopentyl)methoxy)ethanol

Example 81B was synthesized according to the procedure described for Example 72C, substituting Example 81A for Example 72B. MS (APCI) m/z 297.3 (M+H)⁺.

### Example 81C

### (R)-2-(1-((2-((1,4-dioxan-2-yl)methoxy)ethoxy)methyl)cyclopentyl)-4-(dimethoxymethyl)pyrimidine

Example 81C was synthesized according to the procedure described for Example 72E, substituting Example 81B for Example 72C. MS (APCI) m/z 396.3 (M+H)⁺.

### Example 81D

### (R)-2-(1-((2-((1,4-dioxan-2-yl)methoxy)ethoxy)methyl)cyclopentyl)pyrimidine-4-carbaldehyde

Example 81D was synthesized according to the procedure described for Example 29G, substituting Example 81C for Example 29F. MS (APCI) m/z 351.4 (M+H)⁺.

### Example 81E

### (R)-(2-(1-((2-((1,4-dioxan-2-yl)methoxy)ethoxy)methyl)cyclopentyl)pyrimidin-4-yl)methanol

Example 81E was synthesized according to the procedure described for Example 29H, substituting Example 81D for Example 29G. MS (APCI) m/z 353.3 (M+H)⁺.

### Example 81F

### tert-butyl (7R,16R)-19,23-dichloro-10-[(2-{1-[(2-{[(2R)-1,4-dioxan-2-yl]methoxy}ethoxy)methyl]cyclopentyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 81F was synthesized according to the procedure described for Example 291, substituting Example 81E for Example 29H. MS (APCI) m/z 1143.5 (M+H)⁺.

### Example 81G

### (7R,16R)-19,23-dichloro-10-[(2-{1-[(2-{[(2R)-1,4-dioxan-2-yl]methoxy}ethoxy)methyl]cyclopentyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 81G was synthesized according to the procedure described for Example 29J, substituting Example 81F for Example 291. ¹H NMR (500 MHz, dimethylsulfoxide-d₆) δ ppm 8.77-8.68 (m, 2H), 7.41 (d, 1H), 7.23-7.16 (m, 2H), 7.16-7.10 (m, 2H), 6.87 (d, 1H), 6.74 (dd, 1H), 6.24 (dd, 1H), 5.81 (d, 1H), 5.19-5.02 (m, 2H), 4.93-4.85 (m, 1H), 4.53-4.35 (m, 2H), 3.73 (s, 2H), 3.65-3.46 (m, 7H), 3.44-3.36 (m, 2H), 3.28 (dd, 1H), 3.21 (dd, 1H), 3.15 (dd, 1H), 2.95 (dd1H), 2.74-2.62 (m, 2H), 2.48-2.34 (m, 8H), 2.28-2.14 (m, 5H), 1.97 (s, 6H), 1.80-1.72 (m, 2H), 1.70-1.51 (m, 4H). MS (APCI) m/z 1088.6 (M+H)⁺.

### Example 82

### (7R,16R)-19,23-dichloro-1-(4-fluorophenyl)-20,22-dimethyl-16-[(4-methylpiperazin-1-yl)methyl]-10-[(2-{(1s,4s)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 82A

### 2-((1s,4s)-4-(2,5,8,11,14-pentaoxahexadecan-16-yloxy)cyclohexyl)-4-(((tert-butyldiphenylsilyl)oxy)methyl)pyrimidine

To a suspension of NaH (60% oil dispersion, 300 mg) in tetrahydrofuran (5 mL), a solution of Example 70A (200 mg) in tetrahydrofuran (4 mL) was added dropwise at room temperature and the resulting suspension was stirred at room temperature for 1 hour. To the mixture, tetra-n-butylammonium iodide (60 mg) and 16-bromo-2,5,8,11,14-pentaoxahexadecane (430 mg) were added. The mixture was stirred two days at room temperature. The mixture was quenched with aqueous ammonium chloride and extracted with ethyl acetate (300 mL). The organic layer was washed with water and brine, and dried over sodium sulfate. Filtration and evaporation of the solvent gave the crude product which was loaded on a Redi-Sep Gold 40 g column and eluted with 5% methanol in dichloromethane to give the title compound. MS (ESI) m/z 681.3 (M+H)⁺.

### Example 82B

### (2-((1s,4s)-4-(2,5,8,11,14-pentaoxahexadecan-16-yloxy)cyclohexyl)pyrimidin-4-yl)methanol

Example 82B was prepared according to the procedure for Example 57G, substituting Example 82A for Example 57F. MS (ESI) m/z 443.3 (M+H)⁺.

### Example 82C

### tert-butyl (7R,16R)-19,23-dichloro-1-(4-fluorophenyl)-20,22-dimethyl-16-[(4-methylpiperazin-1-yl)methyl]-10-[(2-{(1s,4s)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylate

Example 82C was prepared according to the procedure for Example 57H, substituting Example 82B for Example 57G. MS (ESI) m/z 1234.5 (M+H)⁺.

### Example 82D

### (7R,16R)-19,23-dichloro-1-(4-fluorophenyl)-20,22-dimethyl-16-[(4-methylpiperazin-1-yl)methyl]-10-[(2-{(1s,4s)-4-[(2,5,8,11,14-pentaoxahexadecan-16-yl)oxy]cyclohexyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 82D was prepared according to the procedure for Example 57I, substituting Example 82C for Example 57H. ¹H NMR (400 MHz, dimethylsulfoxide-d₆) δ ppm 8.71 (d, 1H), 7.42 (d, 1H), 7.25-7.14 (m, 2H), 7.14 (ddd, 2H), 6.84 (d, 1H), 6.72 (dd, 1H), 6.19 (dd, 1H), 5.83 (d, 1H), 5.13 (d, 1H), 5.06 (d, 1H), 4.89 (d, 1H), 4.47-4.41 (m, 2H), 3.59 (dd, 1H), 3.57-3.45 (m, 12H), 3.44-3.37 (m, 2H), 3.22 (s, 2H), 2.99-2.90 (m, 1H), 2.90-2.80 (m, 1H), 2.75-2.60 (m, 2H), 2.46 (s, 3H), 2.37 (s, 3H), 2.19 (s, 3H), 1.97 (d, 7H), 1.92-1.80 (m, 3H), 1.71-1.62 (m, 2H), 1.60-1.48 (m, 2H). MS (ESI) m/z 1177.3 (M+H)⁺.

### Example 83

### (7R,16R)-19,23-dichloro-1-(4-fluorophenyl)-10-({2-[(2R)-2-(2,5,8,11,14,17-hexaoxaoctadecan-1-yl) morpholin-4-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 83A

### (R)-(4-(4-(((tert-butyldimethylsilyl)oxy)methyl)pyrimidin-2-yl) morpholin-2-yl)methanol

To a mixture of Example 38A (310 mg) and (R)-morpholin-2-ylmethanol, hydrochloric acid salt (290 mg) in dioxane (5 mL) was added N,N-diisopropylethylamine (830 μL), and the mixture was heated at 90° C. for 5 hours and at 70° C. overnight. The reaction was then heated at 85° C. for 6 hours and concentrated. The reaction was diluted with ethyl acetate and water, and the layers were separated. The organic layer was washed with water and brine, dried over anhydrous sodium sulfate, filtered and concentrated. The residue was purified by silica gel chromatography using an 80 g cartridge to give the title compound. ¹H NMR (400 MHz, CDCl₃) δ ppm 8.32 (d, 1H), 6.77 (d, 1H), 4.59 (s, 2H), 4.55-4.43 (m, 2H), 4.09-3.96 (m, 1H), 3.82-3.56 (m, 4H), 3.14-3.00 (m, 1H), 2.94-2.79 (m, 1H), 1.99 (br s, 1H), 0.95 (s, 9H), 0.11 (s, 6H).

### Example 83B

### (R)-4-(4-(((tert-butyldimethylsilyl)oxy)methyl)pyrimidin-2-yl)-2-(2,5,8,11,14,17-hexaoxaoctadecyl)morpholine

To a solution of Example 83A (200 mg) and 16-bromo-2,5,8,11,14-pentaoxahexadecane (370 mg) in tetrahydrofuran (2.9 mL) was added sodium hydride (47 mg, 60% dispersion in oil), and the reaction was warmed to 40° C. overnight. The reaction was diluted with saturated aqueous ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+12 g gold silica gel column eluting with 50-100% ethyl acetate in dichloromethane to give the title compound. ¹H NMR (400 MHz, dimethylsulfoxide-d₆) δ ppm 8.38 (d, 1H), 6.70 (d, 1H), 4.56 (s, 2H), 4.53-4.44 (m, 1H), 4.43-4.30 (m, 1H), 3.97-3.84 (m, 1H), 3.61-3.37 (m, 24H), 3.23 (s, 3H), 2.99-2.83 (m, 1H), 2.78-2.62 (m, 1H), 0.91 (s, 9H), 0.09 (s, 6H).

### Example 83C

### (R)-(2-(2-(2,5,8,11,14,17-hexaoxaoctadecyl) morpholino)pyrimidin-4-yl)methanol

To a solution of Example 83B (210 mg) in methanol (7.3 mL) was added concentrated hydrochloric acid (164 μL), and the reaction was allowed to stir for 30 minutes. The reaction was concentrated. N,N-Diisopropylethyl amine (0.1 mL) and methanol were added, and the mixture was concentrated. The residue was purified by normal phase MPLC on a Teledyne Isco CombiFlash® Rf+12 g gold silica gel column eluting with 0.5-8% methanol in dichloromethane to give the title compound.

### Example 83D

### (R)-(2-(2-(2,5,8,11,14,17-hexaoxaoctadecyl) morpholino)pyrimidin-4-yl)methyl methanesulfonate

To a solution of Example 83C (50 mg) in dichloromethane (1.1 mL) at 0° C. was added triethylamine (46 μL) followed by methanesulfonyl chloride (10 μL), and the reaction was allowed to warm to room temperature. After one hour, the reaction was concentrated to give the title compound that was used directly in the next step without further purification.

### Example 83E

### tert-butyl (7R,16R)-19,23-dichloro-1-(4-fluorophenyl)-10-({2-[(2R)-2-(2,5,8,11,14,17-hexaoxaoctadecan-1-yl) morpholin-4-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 16N (40 mg) and Example 83D (53 mg) in dimethylformamide (500 μL) was added cesium carbonate (100 mg), and the reaction was allowed to stir overnight. The reaction was diluted with water and extracted with ethyl acetate three times. The combined organic layers were dried over anhydrous sodium sulfate, filtered and concentrated. The residue was purified by normal phase MPLC on a Teledyne Isco CombiFlash® Rf+4 g gold silica gel column eluting with 1-10% methanol in dichloromethane to give the title compound.

### Example 83F

### (7R,16R)-19,23-dichloro-1-(4-fluorophenyl)-10-({2-[(2R)-2-(2,5,8,11,14,17-hexaoxaoctadecan-1-yl) morpholin-4-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 83E (16 mg) in dichloromethane (60 μL) was added trifluoroacetic acid (60 μL), and the reaction was allowed to stir for 5 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-85% over 30 minutes with acetonitrile in water containing 0.01% trifluoroacetic acid) to give the title compound. ¹H NMR (400 MHz, dimethylsulfoxide-d₆) δ ppm 8.73 (s, 1H), 8.35 (d, 1H), 7.25-7.08 (m, 5H), 6.85-6.68 (m, 3H), 6.26-6.17 (m, 1H), 5.86-5.78 (m, 1H), 5.05-4.80 (m, 3H), 4.57-4.33 (m, 4H), 3.96-3.86 (m, 1H), 3.63-3.36 (m, 24H), 3.22 (s, 3H), 3.00-2.88 (m, 2H), 2.79-2.60 (m, 3H), 2.44 (br s, 4H), 2.22 (s, 3H), 2.01-1.92 (m, 6H). MS (ESI) m/z 1192.1 (M−H)⁻.

### Example 84

### (7R,16R)-19,23-dichloro-1-(4-fluorophenyl)-10-({2-[(4R*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 84A

### ethyl 1-methyl-4-(((trifluoromethyl) sulfonyl)oxy)cyclohex-3-enecarboxylate

To a solution of ethyl 1-methyl-4-oxocyclohexanecarboxylate (14 g) in tetrahydrofuran (150 mL) was added potassium hexamethyldisilazide (1 M tetrahydrofuran solution, 129 mL) at −78° C. The reaction mixture was stirred at −78° C. for 1 hour and a solution of 1,1,1-trifluoro-N-phenyl-N-((trifluoromethyl) sulfonyl) methanesulfonamide (34.6 g) in tetrahydrofuran was slowly added at −78° C. The mixture was stirred and allowed to warm to 25° C. in the period of 15 hours. The reaction was quenched with aqueous NH₄Cl solution (100 mL), extracted with ethyl acetate (2×200 mL). The combined organic layer was washed with brine (200 mL), dried over sodium sulfate, filtered, and concentrated. The residue was purified by column chromatography on silica gel (petroleum ether and ethyl acetate=100:1-10:1) to give the title compound.

### Example 84B

### ethyl 1-methyl-4-(4,4,5,5-tetramethyl-1,3,2-dioxaborolan-2-yl)cyclohex-3-enecarboxylate

To a solution of Example 84A (10 g) in 1,4-dioxane (150 mL) was added 4,4,4′,4′,5,5,5′,5′-octamethyl-2,2′-bi(1,3,2-dioxaborolane) (9.15 g), potassium acetate (5.90 g) and PdCl₂(dppf)-CH₂Cl₂ adduct (2.453 g). The mixture was stirred at 80° C. for 12 hours, cooled and filtered. The filtrate was concentrated. The residue was purified by column chromatography on silica gel (petroleum ether:ethyl acetate=100:1 to 10:1) to provide the title compound.

### Example 84C

### ethyl 4-(4-(((tert-butyldimethylsilyl)oxy)methyl)pyrimidin-2-yl)-1-methylcyclohex-3-enecarboxylate

To a mixture of Example 84B (5 g), Example 38A (3.2 g) and K₃PO₄ (8.27 g) in 1,4-dioxane (100 mL) was added Pd(dppf)Cl₂ (0.91 g) under nitrogen atmosphere. The mixture was stirred at 110° C. for 12 hours and was filtered. The filtrate was concentrated. The residue was purified by column chromatography on silica gel (petroleum ether and ethyl acetate=50:1 to 15:1) to give the title compound.

### Example 84D

### (4-(4-(((tert-butyldimethylsilyl)oxy)methyl)pyrimidin-2-yl)-1-methylcyclohex-3-en-1-yl)methanol

To a stirred solution of Example 84C (1 g) in tetrahydrofuran, was added LiBH₄ (0.502 g) at 0° C. The reaction mixture was stirred at 25° C. for 30 hours, diluted with water (20 mL) and extracted with ethyl acetate (3×30 mL). The organic layers were washed with brine (30 mL), dried over sodium sulfate, filtered, and concentrated. The residue which was purified by column chromatography on silica gel (petroleum ether and ethyl acetate=50:1 to 3:1) to provide the title compound.

### Example 84E

### (R)-(4-(4-(((tert-butyldimethylsilyl)oxy)methyl)pyrimidin-2-yl)-1-methylcyclohex-3-en-1-yl)methanol

The enantiomers of Example 84D (4 g) were separated on a Thar SFC 80 preparative SFC (Column: Chiralpak AD-3, 3 μm, 0.46 cm id×5 cm L; Mobile phase: A for SFC CO₂ and B for 2-propanol (0.05% IPAm; Gradient: B in A from 10% to 40% over 3 minutes; Flow rate: 4.0 mL/minute; Wavelength: 220 nm; System Back Pressure: 100 bar) to provide the title compound. The stereochemistry was arbitrarily assigned. ¹H NMR (400 MHz, CDCl₃) δ ppm 8.63 (d, 1H), 7.18 (d, 1H), 7.06 (br s, 1H), 4.61 (s, 2H), 4.45 (t, 1H), 3.13-3.00 (m, 2H), 2.43 (br s, 1H), 2.36-2.20 (m, 1H), 2.06 (br dd, 1H), 1.79 (br d, 1H), 1.49-1.39 (m, 1H), 1.35-1.25 (m, 1H), 0.84-0.80 (m, 1H), 0.82 (s, 9H), 0.75 (s, 3H), 0.00 (s, 6H).

### Example 84F

### (S)-(4-(4-(((tert-butyldimethylsilyl)oxy)methyl)pyrimidin-2-yl)-1-methylcyclohex-3-en-1-yl)methanol

The title compound was obtained from the chiral separation described in Example 84E. ¹H NMR (400 MHz, CDCl₃) δ ppm 8.68 (d, 1H), 7.33 (d, 1H), 7.18 (br s, 1H), 4.76 (s, 2H), 3.49 (t, 1H), 2.66-2.60 (m, 2H), 2.52-2.43 (m, 1H), 2.34-2.20 (m, 1H), 2.13-1.98 (m, 1H), 1.79 (br d, 1H), 1.51-1.39 (m, 1H), 1.37-1.21 (m, 1H), 0.82 (s, 9H), 0.74 (s, 3H), 0.00 (s, 6H).

### Example 84G

### (R)-4-(((tert-butyldimethylsilyl)oxy)methyl)-2-(4-methyl-4-(2,5,8,11,14,17-hexaoxaoctadecyl)cyclohex-1-en-1-yl)pyrimidine

To a solution of Example 84E (0.17 g) in tetrahydrofuran (3 mL) was added NaH (59 mg, 60% in mineral oil) at 0° C. under nitrogen flow. The mixture was stirred for 10 minutes and a solution of 2,5,8,11,14-pentaoxahexadecan-16-yl 4-methylbenzenesulfonate (0.8 g) in tetrahydrofuran (3 mL) was added. The reaction was stirred at 50° C. for 12 hours, cooled, diluted with water (10 mL) and extracted with ethyl acetate (3×10 mL). The combined organic layers were dried over sodium sulfate, filtered, and concentrated to provide the title compound.

### Example 84H

### (R)-(2-(4-methyl-4-(2,5,8,11,14,17-hexaoxaoctadecyl)cyclohex-1-en-1-yl)pyrimidin-4-yl)methanol

To a solution of Example 84G (300 mg) in tetrahydrofuran (5 mL) was added aqueous HCl (4 mL, 2 M) at 0° C. The reaction mixture was stirred at 20° C. for 16 hours under nitrogen atmosphere, neutralized to pH 8 with saturated aqueous NaHCO₃ solution at 0° C., and extracted with ethyl acetate (3×10 mL). The combined organics were dried over sodium sulfate, filtered, and concentrated. The residue was purified by HPLC on a Gilson 281 semi-preparative HPLC system, eluting with 15%-45% acetonitrile in 0.075% TFA water solution to provide the title compound. ¹H NMR (400 MHz, CDCl₃) δ ppm 8.62 (d, 1H), 7.30 (br s, 1H), 7.04 (d, 1H), 4.73 (s, 2H), 3.67-3.63 (m, 14H), 3.62-3.59 (m, 2H), 3.58-3.53 (m, 2H), 3.38 (s, 3H), 3.32-3.19 (m, 2H), 2.73-2.49 (m, 2H), 2.31 (br dd, 1H), 2.04 (br d, 1H), 1.79-1.67 (m, 1H), 1.56 (br dd, 2H), 1.00 (s, 3H).

### Example 84I

### tert-butyl (7R,16R)-19,23-dichloro-1-(4-fluorophenyl)-10-({2-[(4R*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L, replacing Example 101J with Example 84H. MS (ESI) m/z 630.4 (M+H)²⁺.

### Example 84J

### (7R,16R)-19,23-dichloro-1-(4-fluorophenyl)-10-({2-[(4R*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M, replacing Example 101L with Example 841. ¹H NMR (400 MHz, dimethylsulfoxide-d₆) δ ppm 8.78-8.66 (m, 2H), 7.37 (d, 1H), 7.27-7.08 (m, 5H), 6.90-6.69 (m, 2H), 6.23 (dd, 1H), 5.79 (d, 1H), 5.11 (q, 2H), 4.91-4.79 (m, 1H), 4.44 (d, 2H), 3.72-3.38 (m, 29H), 3.26-3.14 (m, 10H), 3.01-2.90 (m, 1H), 2.69 (dt, 3H), 2.28 (d, 8H), 1.97 (d, 7H), 1.60 (dt, 1H), 1.46 (dt, 1H), 0.91 (s, 3H). MS (ESI) m/z 1203.4 (M+H)⁺.

### Example 85

### (7R,16R)-19,23-dichloro-1-(4-fluorophenyl)-10-({2-[(4S*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 85A

### (S)-4-(((tert-butyldimethylsilyl)oxy)methyl)-2-(4-methyl-4-(2,5,8,11,14,17-hexaoxaoctadecyl)cyclohex-1-en-1-yl)pyrimidine

The title compound was prepared as described in Example 84G, replacing Example 84E with Example 84F.

### Example 85B

### (S)-(2-(4-methyl-4-(2,5,8,11,14,17-hexaoxaoctadecyl)cyclohex-1-en-1-yl)pyrimidin-4-yl)methanol

The title compound was prepared as described in Example 84H, replacing Example 84G with Example 85A. ¹H NMR (400 MHz, CDCl₃) δ ppm 8.62 (d, 1H), 7.29 (br t, 1H), 7.04 (d, 1H), 4.73 (s, 2H), 3.68-3.63 (m, 15H), 3.62-3.58 (m, 2H), 3.57-3.52 (m, 2H), 3.38 (s, 3H), 3.32-3.17 (m, 2H), 2.75-2.49 (m, 2H), 2.31 (br dd, 1H), 2.09-1.95 (m, 1H), 1.79-1.65 (m, 1H), 1.62-1.47 (m, 1H), 1.00 (s, 3H).

### Example 85C

### tert-butyl (7R,16R)-19,23-dichloro-1-(4-fluorophenyl)-10-({2-[(4S*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L, replacing Example 101J with Example 85B. MS (ESI) m/z 630.4 (M+H)²⁺.

### Example 85D

### (7R,16R)-19,23-dichloro-1-(4-fluorophenyl)-10-({2-[(4S*)-4-(2,5,8,11,14,17-hexaoxaoctadecan-1-yl)-4-methylcyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M, replacing Example 101L with Example 85C. ¹H NMR (400 MHz, dimethylsulfoxide-d₆) δ ppm 8.78-8.65 (m, 2H), 7.37 (d, 1H), 7.28-7.08 (m, 5H), 6.90-6.69 (m, 2H), 6.23 (dd, 1H), 5.79 (d, 1H), 5.11 (q, 2H), 4.85 (d, 1H), 4.44 (d, 2H), 3.74-3.45 (m, 21H), 3.41 (dd, 2H), 3.25-3.10 (m, 5H), 3.03-2.89 (m, 1H), 2.68 (t, 2H), 2.28 (d, 15H), 1.97 (d, 8H), 1.69-1.40 (m, 2H), 0.91 (s, 3H). MS (ESI) m/z 1203.5 (M+H)⁺.

### Example 86

### (7R,16R)-19,23-dichloro-10-({2-[(4S*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 86A

### 8-fluoro-8-((2-(2-methoxyethoxy)ethoxy)methyl)-1,4-dioxaspiro[4.5]decane

The title compound was prepared as described in Example 101E, replacing 2-(2-(2-methoxyethoxy)ethoxy)ethyl 4-methylbenzenesulfonate with 2-(2-methoxyethoxy)ethyl 4-methylbenzenesulfonate.

### Example 86B

### 4-fluoro-4-((2-(2-methoxyethoxy)ethoxy)methyl)cyclohexanone

The title compound was prepared as described in Example 101F, replacing Example 101E with Example 86A.

### Example 86C

### 4-fluoro-4-((2-(2-methoxyethoxy)ethoxy)methyl)cyclohex-1-en-1-yl trifluoromethanesulfonate

The title compound was prepared as described in Example 101G, replacing Example 101F with Example 86B.

### Example 86D

### 2-(4-fluoro-4-((2-(2-methoxyethoxy)ethoxy)methyl)cyclohex-1-en-1-yl)-4,4,5,5-tetramethyl-1,3,2-dioxaborolane

The title compound was prepared as described in Example 101H, replacing Example 101G with Example 86C.

### Example 86E

### (2-(4-fluoro-4-((2-(2-methoxyethoxy)ethoxy)methyl)cyclohex-1-en-1-yl)pyrimidin-4-yl)methanol

The title compound was prepared as described in Example 101I, replacing Example 101H with Example 86D.

### Example 86F

### (S)-(2-(4-fluoro-4-((2-(2-methoxyethoxy)ethoxy)methyl)cyclohex-1-en-1-yl)pyrimidin-4-yl)methanol

The title compound was prepared as described in Example 101J, replacing Example 101I with Example 86E. ¹H NMR (400 MHz, CDCl₃) δ ppm 8.64 (d, 1H), 7.23 (br s, 1H), 7.07 (d, 1H), 4.74 (s, 2H), 3.77-3.73 (m, 2H), 3.73-3.64 (m, 6H), 3.62 (s, 1H), 3.58-3.54 (m, 2H), 3.39 (s, 3H), 2.85-2.75 (m, 2H), 2.63 (br s, 1H), 2.61-2.51 (m, 1H), 2.19-2.11 (m, 1H), 1.99-1.83 (m, 1H).

### Example 86G

### (R)-(2-(4-fluoro-4-((2-(2-methoxyethoxy)ethoxy)methyl)cyclohex-1-en-1-yl)pyrimidin-4-yl)methanol

The title compound was prepared as described in Example 101J, replacing Example 101I with Example 86E. ¹H NMR (400 MHz, CDCl₃) δ ppm 8.64 (d, 1H), 7.24 (br s, 1H), 7.07 (d, 1H), 4.74 (s, 2H), 3.77-3.74 (m, 2H), 3.73-3.65 (m, 6H), 3.63 (s, 1H), 3.57-3.55 (m, 2H), 3.39 (s, 3H), 2.80 (br s, 2H), 2.66-2.54 (m, 2H), 2.18-2.11 (m, 1H), 1.98-1.84 (m, 1H).

### Example 86H

### tert-butyl (7R,16R)-19,23-dichloro-10-({2-[(4S*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L, replacing Example 101J with Example 86F. MS (ESI) m/z 1131.5 (M+H)⁺.

### Example 86I

### (7R,16R)-19,23-dichloro-10-({2-[(4S*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M, replacing Example 101L with Example 86H. ¹H NMR (500 MHz, dimethylsulfoxide-d₆) δ ppm 8.75 (d, 2H), 7.40 (d, 1H), 7.24-7.17 (m, 2H), 7.14 (td, 3H), 6.85 (d, 1H), 6.76 (dd, 1H), 6.25 (dd, 1H), 5.79 (d, 1H), 5.17 (d, 1H), 5.09 (d, 1H), 4.86 (p, 1H), 4.45 (d, 2H), 3.68-3.56 (m, 6H), 3.56-3.51 (m, 6H), 3.46-3.40 (m, 2H), 3.24 (s, 3H), 3.01-2.93 (m, 1H), 2.70 (qd, 4H), 2.54 (s, 3H), 2.31 (s, 3H), 2.04 (t, 1H), 2.00 (s, 3H), 1.95 (s, 3H), 1.79 (dt, 1H). MS (ESI) m/z 1123.7 (M+H)⁺.

### Example 87

### (7R,16R)-19,23-dichloro-10-({2-[(4R*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 87A

### tert-butyl (7R,16R)-19,23-dichloro-10-({2-[(4R*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 86G. MS (ESI) m/z 1133.5 (M+H)⁺.

### Example 87B

### (7R,16R)-19,23-dichloro-10-({2-[(4R*)-4-fluoro-4-{[2-(2-methoxyethoxy)ethoxy]methyl}cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 87A. ¹H NMR (400 MHz, dimethylsulfoxide-d₆) δ ppm 8.79-8.69 (m, 2H), 7.41 (d, 1H), 7.26-7.08 (m, 5H), 6.89-6.68 (m, 2H), 6.21 (d, 1H), 5.81 (d, 1H), 5.12 (q, 2H), 4.87 (s, 1H), 4.44 (d, 2H), 3.67-3.51 (m, 9H), 3.49-3.41 (m, 10H), 3.24 (s, 3H), 2.96 (d, 1H), 2.82-2.61 (m, 2H), 2.36 (s, 4H), 2.46-2.25 (m, OH), 2.18 (s, 3H), 1.97 (d, 7H). MS (ESI) m/z 1077.4 (M+H)⁺.

### Example 88

### (7R,16R)-19,23-dichloro-1-(4-fluorophenyl)-10-({2-[1-(2,5,8,11,14,17-hexaoxaoctadecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 88A

### 1-(((tert-butyldimethylsilyl)oxy)methyl)cyclohexanecarbonitrile

To a flask containing 1-(hydroxymethyl)cyclohexane-1-carbonitrile (2.200 g) in dichloromethane (33 mL) was added tert-butyldimethylsilyl chloride (3097 mg) followed by imidazole (2.152 g). The resulting mixture was stirred for 4 hours. The mixture was then concentrated and purified by silica gel flash chromatography on AnaLogix IntelliFlash²⁸⁰ system eluting with 0-20% ethyl acetate in hexanes to give the title compound. MS (ESI) m/z 254.4 (M+H)⁺.

### Example 88B

### 2-(1-(((tert-butyldimethylsilyl)oxy)methyl)cyclohexyl)-4-(dimethoxymethyl)pyrimidine

A solution of trimethylaluminum (12.07 mL, 2M in toluene) was slowly added to a stirred suspension of ammonium chloride (1292 mg) in toluene (34.0 mL) at 0° C. After the addition, the ice water was removed and the mixture was stirred for 2 hours until gas evolution had ceased. Next, 1-(((tert-butyldimethylsilyl)oxy)methyl)cyclohexanecarbonitrile (3400 mg) was added as a toluene (17 mL) solution. The resulting mixture was stirred at 80° C. for 12 hours. The reaction mixture was cooled with an ice water bath and was quenched carefully with 5 mL of methanol and stirred for 2 hours. The material was removed through filtration and washed with methanol. The combined filtrate was concentrated to afford the crude amidine, which was taken up in ethanol (20 mL) and to this was added a 21% ethanol solution of sodium ethoxide (26.1 g) which warmed the reaction mildly. The thick mixture was heated at 80° C. for 16 hours and was concentrated. Saturated aqueous sodium bicarbonate was added (150 mL) and the mixture was stirred for 2 minutes. The mixture was extracted with three portions of dichloromethane. The organic layers were combined and the resulting solution was dried over anhydrous magnesium sulfate, filtered and concentrated. The crude product was purified by silica gel flash chromatography on AnaLogix IntelliFlash²⁸⁰ system eluting with 5-80% ethyl acetate in heptanes to give the title compound. MS (ESI) m/z 381.2 (M+H)⁺.

### Example 88C

### (1-(4-(dimethoxymethyl)pyrimidin-2-yl)cyclohexyl)methanol

To a solution of Example 88B (1400 mg) in tetrahydrofuran (14 mL) was added tetrabutylammonium fluoride (7.36 mL). The mixture was stirred for 1 hour. The mixture was concentrated and purified by silica gel flash chromatography on AnaLogix IntelliFlash²⁸⁰ system eluting with 25-80% ethyl acetate in hexanes to give the title compound. LC/MS (APCI) m/z 267.36 (M+H)⁺.

### Example 88D

### 2-(1-(2,5,8,11,14,17-hexaoxaoctadecyl)cyclohexyl)-4-(dimethoxymethyl)pyrimidine

To a stirring solution of Example 88C (200 mg) and M-PEG5-bromide (473 mg) in acetonitrile (6 mL) was slowly added sodium hydride (36.0 mg) and the mixture was stirred at 45° C. for 1 day. A few drops of saturated aqueous ammonium chloride were added. The mixture was concentrated onto silica gel and was purified by silica gel flash chromatography (solvent A=3:1 ethyl acetate:ethanol; solvent B=heptanes, eluting with 30-100% A to B) to give the title compound. MS (ESI) m/z 501.3 (M+H)⁺.

### Example 88E

### 2-(1-(2,5,8,11,14,17-hexaoxaoctadecyl)cyclohexyl)pyrimidine-4-carbaldehyde

To a stirring solution of Example 88D (236 mg) in tetrahydrofuran (4 mL) was slowly added aqueous hydrochloric acid solution (2.83 mL) and the mixture was stirred at 55° C. for 5 hours. The mixture was cooled to room temperature and poured into a separatory funnel containing saturated aqueous sodium bicarbonate. The mixture was extracted three times with dichloromethane. The organic layer was dried over anhydrous sodium sulfate, filtered and concentrated. The crude title compound was carried through the next step without further purification. LC/MS (APCI) m/z 455.0 (M+H)⁺.

### Example 88F

### (2-(1-(2,5,8,11,14,17-hexaoxaoctadecyl)cyclohexyl)pyrimidin-4-yl)methanol

To a solution of Example 88E (214 mg) in tetrahydrofuran (3 mL) was added sodium borohydride (35.6 mg) in one portion followed by methanol (1.3 mL). The mixture was stirred for 20 minutes. The mixture was quenched by careful addition of 3 mL of saturated aqueous ammonium chloride solution, stirred for 15 minutes and poured into a separatory funnel containing 8 mL of water. The mixture was extracted with 3 portions of dichloromethane. The combined organic layers were dried over anhydrous magnesium sulfate, filtered and concentrated onto silica gel. Purification by flash chromatography on an AnaLogix IntelliFlash²⁸⁰ system (solvent A=3:1 ethyl acetate:ethanol; solvent B=heptane, eluting with 30-100% A to B) afforded the title compound. MS (ESI) m/z 457.3 (M+H)⁺.

### Example 88G

### tert-butyl (7R,16R)-19,23-dichloro-1-(4-fluorophenyl)-10-({2-[1-(2,5,8,11,14,17-hexaoxaoctadecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (50 mg), Example 88F (56.4 mg) and triphenylphosphine (34.0 mg). The vial was capped with a septum then evacuated and backfilled with nitrogen. Toluene (0.6 mL) was added and the mixture was cooled with an ice bath. Di-tert-butyl azodicarboxylate (28.4 mg) was added in one solid portion. The vial was capped with a septum, evacuated and backfilled with nitrogen twice. The mixture was stirred at 0° C. for 10 minutes and the cooling bath was removed and the mixture was allowed to stir overnight. The mixture was concentrated and the residue was purified by silica gel flash chromatography on AnaLogix IntelliFlash²⁸⁰ system eluting with 0-20% methanol in dichloromethane to give the title compound. LC/MS (ESI) m/z 1247.5 (M+H)⁺.

### Example 88H

### (7R,16R)-19,23-dichloro-1-(4-fluorophenyl)-10-({2-[1-(2,5,8,11,14,17-hexaoxaoctadecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 88G for Example 26E in Example 26F. ¹H NMR (400 MHz, dimethylsulfoxide-d₆) δ ppm 8.75 (d, 1H), 8.70 (s, 1H), 7.42 (d, 1H), 7.25-7.07 (m, 4H), 6.84 (d, 1H), 6.69 (dd, 1H), 6.18 (dd, 1H), 5.85 (d, 1H), 5.09 (q, 2H), 4.91 (q, 1H), 4.43 (d, 2H), 3.61-3.25 (m, 26H), 3.22 (s, 3H), 2.98-2.89 (m, 1H), 2.67 (qd, 3H), 2.45-2.25 (m, 6H), 2.19 (s, 3H), 1.99 (s, 3H), 1.93 (s, 3H), 1.62-1.14 (m, 8H). MS (ESI) m/z 1191.3 (M+H)⁺.

### Example 89

### (7R,16R)-19,23-dichloro-1-(4-fluorophenyl)-20,22-dimethyl-16-[(4-methylpiperazin-1-yl)methyl]-10-({2-[(2,5,8,11-tetraoxatridecan-13-yl)oxy]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 89A

### methyl 2-((2,5,8,11-tetraoxatridecan-13-yl)oxy)pyrimidine-4-carboxylate

A 100 mL three neck flask was charged with NaH (55%, 130 mg) and tetrahydrofuran (2 mL). At 5° C. tetraethyleneglycol monomethylether (530 mg) dissolved in tetrahydrofuran (2 mL) was added dropwise and the mixture was stirred for 1 hour at 5° C. A solution of methyl 2-chloropyrimidine-4-carboxylate (390 mg) in tetrahydrofuran (4 mL) was added at 5° C. and stirring was continued at ambient temperature for 2 hours. Tetrahydrofuran and water were added (10:1, 10 mL), and the mixture extracted three times with dichloromethane. The combined organic layers washed with brine, dried over magnesium sulfate, filtered and concentrated in vacuo. Purification by chromatography using an ISCO CombiFlash® Companion MPLC (12 g RediSep® Gold column, eluting with 0-50% dichloromethane/methanol) followed by treatment with n-pentane, filtration, concentration and purification by ISCO CombiFlash® Companion MPLC (15 g Chromabond® RP-C18 column, eluting with 0-100% water/methanol) gave the title compound. MS (APCI) m/z 344.2 (M+H)⁺.

### Example 89B

### (2-((2,5,8,11-tetraoxatridecan-13-yl)oxy)pyrimidin-4-yl)methanol

To a solution of Example 89A (48 mg) in methanol (2 mL) sodium borohydride (11 mg) was added in two portions and the reaction was stirred at ambient temperature for 1 hour. Water (0.2 mL) was added. The mixture was concentrated in vacuo, dichloromethane (15 mL) and water (1 mL) were added, and the mixture was separated via Chromabond® PTS cartridge. The organic layer was concentrated to give the title compound. MS (APCI) m/z 317.2 (M+H)⁺.

### Example 89C

### tert-butyl (7R,16R)-19,23-dichloro-1-(4-fluorophenyl)-20,22-dimethyl-16-[(4-methylpiperazin-1-yl)methyl]-10-({2-[(2,5,8,11-tetraoxatridecan-13-yl)oxy]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

A 10 mL round-bottomed flask was charged with Example 16N (40 mg), Example 89B (34 mg), triphenylphosphine (51 mg) and (E)-N¹,N¹,N²,N²-tetramethyldiazene-1,2-dicarboxamide (34 mg) and degassed for 15 minutes with nitrogen. Tetrahydrofuran (1 mL) and toluene (1 mL), both degassed for 30 minutes with nitrogen, were added via syringe and the reaction mixture stirred for 3 days at ambient temperature. Telos Bulk Sorbent was added, and the mixture was concentrated to dryness. The material was directly subjected to chromatography using an ISCO CombiFlash® Companion MPLC (12 g RediSep® Gold column, eluting with 0-50% dichloromethane/methanol) to give the title compound. MS (APCI) m/z 1107.4 (M+H)⁺.

### Example 89D

### (7R,16R)-19,23-dichloro-1-(4-fluorophenyl)-20,22-dimethyl-16-[(4-methylpiperazin-1-yl)methyl]-10-({2-[(2,5,8,11-tetraoxatridecan-13-yl)oxy]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

To Example 89C (62 mg) in dichloromethane (1.5 mL) was added trifluoroacetic acid (0.35 mL), and the reaction stirred at ambient temperature. The mixture was concentrated in vacuo and purified by HPLC (XBridge C8 19×150 mm 5 μm column, gradient 5-100% acetonitrile+0.2% ammonium hydroxide in water+0.1% ammonium hydroxide) providing the title compound. ¹H NMR (600 MHz, dimethylsulfoxide-d₆) δ ppm 8.70 (s, 1H), 8.58 (d, 1H), 7.25 (d, 1H), 7.19 (t, 2H), 7.12 (m, 1H), 6.79 (m, 1H), 6.70 (m, 1H), 6.17 (m, 1H), 5.83 (m, 1H), 5.09 (d, 1H), 5.01 (d, 1H), 4.89 (m, 1H), 4.43 (m, 4H), 3.75 (m, 2H), 3.58 (m, 2H), 3.53 (m, 2H), 3.50 (m, 6H), 3.41 (m, 2H), 3.22 (s, 3H), 2.92 (dd, 1H), 2.67 (m, 2H), 2.55-2.45 (m, 4H), 2.34 (s, 3H), 2.17 (s, 3H), 2.06-1.86 (s, 6H). MS (APCI) m/z 1051.4 (M+H)⁺.

### Example 90

### (7R,16R)-19,23-dichloro-1-cyclohexyl-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 90A

### tert-butyl (7R,16R)-10-(benzyloxy)-19,23-dichloro-1-(cyclohex-1-en-1-yl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 130L (400 mg), 1,1′-bis(diphenylphosphino)ferrocene-palladium (ii) dichloride dichloromethane complex (35 mg), 1-cyclohexen-yl-boronic acid pinacol ester (160 mg), and cesium carbonate were combined under an argon atmosphere in dioxane/water (degassed, 4 mL/9 mL). The reaction mixture was heated to 90° C. and stirred for 45 minutes. The reaction mixture was partitioned between water and ethyl acetate. The aqueous phase was extracted with ethyl acetate twice. The combined organic layer was washed with brine, dried over anhydrous magnesium sulfate, filtered and concentrated. The residue was purified on a silica gel column (12 g, 0-10% methanol in dichloromethane). The desired fractions were combined and the solvents were removed under reduced pressure to provide the title compound. MS (ESI) m/z 885.3 (M+H)⁺.

### Example 90B

### tert-butyl (7R,16R)-19,23-dichloro-1-cyclohexyl-10-hydroxy-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

In a 20 mL tinyclave reactor. Example 90A was dissolved in tetrahydrofuran (12 mL), and palladium on carbon (68 mg, 100%, wet) was added under nitrogen atmosphere. The reactor was flushed with hydrogen four times and set under pressure of 50 psi (3.45 bar). The reaction mixture was stirred at room temperature for 22 hours. Additional palladium on carbon (66 mg, 10%, wet) was added to the reaction mixture. The reactor was flushed with hydrogen four times and set under pressure of ca.52 psi. The mixture was stirred at room temperature for additional 23 hours. The catalyst was filtered off and the filtrate was concentrated. The residue was purified on silica gel column (12 g, 0-100% methanol in dichloromethane). The desired fractions were combined and the solvents were removed under reduced pressure to provide the title compound. ¹H NMR (600 MHz, dimethylsulfoxide-d6) δ ppm 9.06 (s, 1H), 8.65 (s, 1H), 6.70 (dd, 1H), 6.64 (d, 1H), 5.94 (dd, 1H), 5.49 (d, 1H), 4.68 (q, 1H), 4.50-4.46 (m, 1H), 4.40 (d, 1H), 3.50 (dd, 1H), 2.71-2.65 (m, 2H), 2.57 (d, 1H), 2.51-2.25 (m, 9H), 2.17 (m, 3H), 2.02 (s, 3H), 1.99 (s, 3H), 1.83 (d, 1H), 1.74-1.58 (m, 4H), 1.49-1.42 (m, 1H), 1.39-1.32 (m, 1H), 1.24-1.08 (m, 3H), 1.07 (s, 9H). MS (ESI) m/z 797.3 (M+H)⁺.

### Example 90C

### tert-butyl (7R,16R)-19,23-dichloro-1-cyclohexyl-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 90B (22 mg), Example 13C (35 mg), triphenylphosphine (37 mg), and N,N,N′,N′-tetramethylazodicarboxamide were combined under argon atmosphere. Tetrahydrofuran (0.7 mL) and toluene (0.7 mL) were added. The reaction mixture was stirred at room temperature for 5 minutes. The mixture was heated to 50° C. and stirred for 6 hours. All volatiles were evaporated and the residue was partitioned between water and dichloromethane. The organic layer was washed with water and aqueous sodium bicarbonate solution. The combined aqueous layers were extracted with dichloromethane twice. The combined organic extracts were dried over magnesium sulfate, filtrated, and concentrated. Purification was performed on a silica gel column (4 g, 0-10% methanol in dichloromethane). The desired fractions were combined and the solvents were removed under reduced pressure to provide the title compound. MS (ESI) m/z 1127.4 (M+H)⁺.

### Example 90D

### (7R,16R)-19,23-dichloro-1-cyclohexyl-10-{[2-(4-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 90C (13 mg) was dissolved in dichloromethane. Trifluoroacetic acid (36 μL) was added and the mixture was stirred at room temperature overnight. The solvent was evaporated at room temperature. The residue was diluted with dichloromethane and washed with aqueous saturated sodium bicarbonate solution. The aqueous layer was extracted with dichloromethane twice. The combined organic phases were dried over magnesium sulfate, filtrated, and concentrated. Purification was performed on a silica gel column (4 g, 0-20% methanol in dichloromethane). The desired fractions were combined and the solvents were removed under reduced pressure to provide the title compound. ¹H NMR (600 MHz, dimethylsulfoxide-d6) δ ppm 8.82 (d, 1H), 8.66 (m, 1H), 8.35-8.33 (m, 2H), 7.43 (d, 1H), 7.09-7.06 (m, 2H), 6.89 (d, 1H), 6.77 (d, 1H), 6.26 (m, 1H), 5.79 (m, 1H), 5.24 (d, 1H), 5.16 (d, 1H), 4.87 (m, 1H), 4.53-4.47 (m, 2H), 4.18-4.17 (m, 2H), 3.78-3.77 (m, 2H), 3.61-3.59 (m, 2H), 3.55-3.51 (m, 5H), 3.44-3.42 (m, 2H), 3.33 (s, 3H), 2.90 (d, 1H), 2.73-2.67 (m, 2H), 2.54-2.30 (m, 8H), 2.22-2.18 (m, 1H), 2.15 (s, 3H), 2.03 (s, 3H), 1.91 (s, 3H), 1.78-1.75 (m, 1H), 1.72-1.66 (m, 3H), 1.59-1.55 (m, 1H), 1.45-1.32 (m, 2H), 1.19-1.08 (m, 3H). MS (ESI) m/z 1071.3 (M+H)⁺.

### Example 91

### (7R,16R)-19,23-dichloro-10-({2-[4-({2-[(1,4-dioxan-2-yl)methoxy]ethoxy}methyl)-4-fluoropiperidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 91A

### (1-(4-(((tert-butyldimethylsilyl)oxy)methyl)pyrimidin-2-yl)-4-fluoropiperidin-4-yl)methanol

A solution of (4-fluoropiperidin-4-yl)methanol, hydrochloric acid (400 mg), Example 38A (510 mg) and N,N-diisopropylethylamine (1.7 mL) in acetonitrile (4.9 mL) was heated to 80° C. for 6 hours and stirred overnight at room temperature. The reaction was diluted with water and extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 0-40% ethyl acetate in heptanes to give the title compound. ¹H NMR (400 MHz, dimethylsulfoxide-d₆) δ ppm 8.35 (d, 1H), 6.65 (d, 1H), 5.02-4.91 (m, 1H), 4.54 (s, 2H), 4.48-4.33 (m, 2H), 3.50-3.37 (m, 2H), 3.28-3.11 (m, 2H), 1.84-1.45 (m, 4H), 0.91 (s, 9H), 0.09 (s, 6H).

### Example 91B

### 2-(4-((2-(allyloxy)ethoxy)methyl)-4-fluoropiperidin-1-yl)-4-(((tert-butyldimethylsilyl)oxy)methyl)pyrimidine

To a solution of Example 91A (310 mg) in tetrahydrofuran (8.7 mL) at 0° C. was added sodium hydride (70 mg, 60% oil dispersion), and the reaction was allowed to stir for 1 hour as it warmed to room temperature. Tetrabutylammonium iodide (320 mg) and 3-(2-bromoethoxy) prop-1-ene (430 mg) were added, and the reaction was allowed to stir at room temperature overnight. The reaction was quenched with saturated aqueous ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+12 g gold silica gel column eluting with 0-35% ethyl acetate in heptanes to give the title compound. ¹H NMR (400 MHz, dimethylsulfoxide-d₆) δ ppm 8.35 (d, 1H), 6.65 (d, 1H), 5.95-5.77 (m, 1H), 5.28-5.18 (m, 1H), 5.16-5.07 (m, 1H), 4.54 (s, 2H), 4.46-4.34 (m, 2H), 3.99-3.90 (m, 2H), 3.62-3.45 (m, 6H), 3.28-3.15 (m, 2H), 1.88-1.74 (m, 2H), 1.72-1.51 (m, 2H), 0.91 (s, 9H), 0.09 (s, 6H).

### Example 91C

### 3-(2-((1-(4-(((tert-butyldimethylsilyl)oxy)methyl)pyrimidin-2-yl)-4-fluoropiperidin-4-yl)methoxy)ethoxy)propane-1,2-diol

To a solution of Example 91B (185 mg) in t-butanol (2.1 mL) and water (2.1 mL) at 0° C. was added AD-Mix alpha (1 g), and the reaction was stirred for 4 hours at 0° C. The reaction was warmed to room temperature and stirred overnight. The reaction was quenched with solid sodium sulfite and extracted with ethyl acetate three times. The combined organic layers were washed with brine, dried over anhydrous sodium sulfate, filtered and concentrated to give the title compound as a mixture of isomers that was used in the next step without further purification.

### Example 91D

### Phenyl(Vinyl)Selane

To a solution of 1,2-diphenyldiselane (7 g) in tetrahydrofuran (75 mL) at 0° C. was added vinylmagnesium bromide (49.3 mL, 1 M in tetrahydrofuran) over 25 minutes. The reaction was allowed to warm to room temperature and stir overnight. The reaction was slowly diluted with water with water bath cooling and extracted with ethyl acetate three times. The combined organic layers were dried over anhydrous sodium sulfate, filtered and concentrated. The residue was purified by normal phase MPLC on a Teledyne Isco CombiFlash® Rf+120 g gold silica gel column, eluting with heptanes to give the title compound. ¹H NMR (500 MHz, CDCl₃) δ ppm 7.57-7.49 (m, 2H), 7.36-7.27 (m, 3H), 6.91-6.79 (m, 1H), 5.83-5.75 (m, 1H), 5.60-5.50 (m, 1H).

### Example 91E

### (Vinylselenonyl)Benzene

To a solution of Example 91D (1.2 g) in tetrahydrofuran (120 mL) was added potassium phosphate dibasic (3.4 g) and magnesium monoperoxyphthalate hexahydrate (8.1 g), and the reaction was allowed to stir for 3 hours. The reaction was diluted with ethyl acetate and washed with 10% aqueous sodium carbonate followed by brine. The organic layer was dried over anhydrous sodium sulfate, filtered and concentrated to give the title compound that was used in the next step without further purification. ¹H NMR (500 MHz, CDCl₃) δ ppm 8.01-7.91 (m, 2H), 7.74-7.60 (m, 3H), 7.08-6.90 (m, 1H), 6.76-6.68 (m, 1H), 6.48-41 (m, 1H).

### Example 91F

### 2-(4-((2-((1,4-dioxan-2-yl)methoxy)ethoxy)methyl)-4-fluoropiperidin-1-yl)-4-(((tert-butyldimethylsilyl)oxy)methyl)pyrimidine

To a solution of Example 91C (200 mg) in dichloromethane (2.8 mL) at room temperature was added sodium hydride (30 mg, 60% oil dispersion), and the reaction was allowed to stir for 10 minutes. A solution of Example 91E (400 mg) in dichloromethane (1.4 mL) was added, and the reaction was allowed to stir for 4 hours. The reaction was quenched with saturated ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+4 g gold silica gel column eluting with 0-45% ethyl acetate in dichloromethane to give the title compound. ¹H NMR (400 MHz, dimethylsulfoxide-d₆) δ ppm 8.36 (d, 1H), 6.66 (d, 1H), 4.54 (s, 2H), 4.46-4.32 (m, 2H), 3.73-3.33 (m, 14H), 3.29-3.15 (m, 3H), 1.88-1.73 (m, 2H), 1.72-1.49 (m, 2H), 0.91 (s, 9H), 0.09 (s, 6H).

### Example 91G

### (2-(4-((2-((1,4-dioxan-2-yl)methoxy)ethoxy)methyl)-4-fluoropiperidin-1-yl)pyrimidin-4-yl)methanol

To a solution of Example 91F (160 mg) in tetrahydrofuran (1.1 mL) and methanol (540 μL) at room temperature was added cesium fluoride (250 mg), and the reaction was allowed to stir overnight. The reaction was concentrated, and the residue was treated with heptane to remove non-polar material. The remaining material was taken up in ethyl acetate, filtered over diatomaceous earth and concentrated. The residue was purified by normal phase MPLC on a Teledyne Isco CombiFlash® Rf+4 g gold silica gel column eluting with 3-10% methanol in dichloromethane to give the title compound. ¹H NMR (400 MHz, dimethylsulfoxide-d₆) δ ppm 8.33 (d, 1H), 6.71 (d, 1H), 5.44-5.34 (m, 1H), 4.47-4.30 (m, 4H), 3.74-3.35 (m, 14H), 3.29-3.14 (m, 3H), 1.87-1.74 (m, 2H), 1.72-1.50 (m, 2H).

### Example 91H

### tert-butyl (7R,16R)-19,23-dichloro-10-({2-[4-({2-[(1,4-dioxan-2-yl)methoxy]ethoxy}methyl)-4-fluoropiperidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A vial containing Example 91G (54 mg), Example 16N (38 mg), triphenylphosphine (37 mg) and N,N,N′,N′-tetramethylazodicarboxamide (24 mg) in toluene (120 μL) and tetrahydrofuran (120 μL) was allowed to stir at 50° C. for 6 hours. The residue was purified by normal phase MPLC on a Teledyne Isco CombiFlash® Rf+4 g gold silica gel column eluting with 1-9% methanol in dichloromethane to give the title compound.

### Example 91I

### (7R,16R)-19,23-dichloro-10-({2-[4-({2-[(1,4-dioxan-2-yl)methoxy]ethoxy}methyl)-4-fluoropiperidin-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 91H (68 mg) in dichloromethane (290 μL) was added trifluoroacetic acid (290 μL), and the reaction was allowed to stir for 4 hours. The reaction was concentrated under a stream of nitrogen and taken up in water and acetonitrile. The mixture was purified by RP-HPLC on a Gilson PLC 2020 using a Luna™ column (250×50 mm, 10 mm) (5-80% over 30 minutes with acetonitrile in water containing 10 mM ammonium acetate) to give the title compound. ¹H NMR (400 MHz, dimethylsulfoxide-d₆) δ ppm 8.73 (s, 1H), 8.33 (d, 1H), 7.27-7.07 (m, 5H), 6.81 (d, 1H), 6.76-6.68 (m, 2H), 6.27-6.16 (m, 1H), 585-5.76 (m, 1H), 5.03-4.80 (m, 3H), 4.53-4.34 (m, 4H), 3.71-3.16 (m, 17H), 2.98-2.88 (m, 1H), 2.76-2.59 (m, 2H), 2.46 (br s, 4H), 2.23 (s, 3H), 2.03-1.93 (m, 6H), 1.88-1.75 (m, 2H), 1.73-1.51 (m, 2H). MS (ESI) m/z 1120.1 (M−H)⁻.

### Example 92

### (7R,16R)-19,23-dichloro-10-({2-[(1r,4r)-4-{2-[(1,4-dioxan-2-yl)methoxy]ethoxy}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 92A

### 2-chloro-4-(((2-(trimethylsilyl)ethoxy)methoxy)methyl)pyrimidine

To a solution of (2-chloropyrimidin-4-yl)methanol (12 g) in dichloromethane (300 mL) was added N,N-diisopropylethylamine (20 mL) followed by chloromethyl 2-trimethylsilylethyl ether (15.22 g). The mixture was stirred under nitrogen at room temperature overnight. The mixture was diluted with water (100 mL), and ethyl acetate (600 mL). The organic layer was separated and washed with water and brine and dried over sodium sulfate. Filtration and evaporation of the solvent gave crude product which was loaded on a Redi-Sep Gold 220 g column and eluted with 10% ethyl acetate in heptane to give the title compound. MS (ESI) m/z 275.2 (M+H)⁺.

### Example 92B

### 2-(1,4-dioxaspiro[4.5]dec-7-en-8-yl)-4-(((2-(trimethylsilyl)ethoxy)methoxy)methyl)pyrimidine

To a solution of 4,4,5,5-tetramethyl-2-(1,4-dioxaspiro[4.5]dec-7-en-8-yl)-1,3,2-dioxaborolane (16.27 g) and Example 92A (16.8 g) in tetrahydrofuran (220 mL) was added Pd(Ph₃P)₄ (3.53 g) and aqueous saturated sodium bicarbonate (120 mL). The mixture was stirred under nitrogen at 70° C. overnight. The mixture was concentrated under vacuum and the residue was diluted with water (120 mL) and ethyl acetate (800 mL). The organic layer was separated, washed with water and brine and dried over sodium sulfate. Filtration and evaporation of the solvent gave crude product which was loaded on a Redi-Sep Gold 330 g column and eluted with 20% ethyl acetate in heptane to give the title compound. MS (ESI) m/z 379.1 (M+H)⁺.

### Example 92C

### 2-(1,4-dioxaspiro[4.5]decan-8-yl)-4-(((2-(trimethylsilyl)ethoxy)methoxy)methyl)pyrimidine

To a solution of Example 92B (21 g) in tetrahydrofuran (120 mL) was added Pd/C (10% 1.5 g). The mixture was stirred under hydrogen (25 psi) at room temperature for 4 hours. The mixture was filtered and concentrated under vacuum to give the title compound. MS (ESI) m/z 381.2 (M+H)⁺.

### Example 92D

### 4-(4-(((2-(trimethylsilyl)ethoxy)methoxy)methyl)pyrimidin-2-yl)cyclohexanone

To a solution of Example 92 C (12 g) in acetone (70 mL) and water (30 mL) was added pyridinium p-toluenesulfonate (1.5 g). The mixture was stirred at reflux for 16 hours. The mixture was concentrated under vacuum and the residue was diluted with water (120 mL) and ethyl acetate (400 mL). The organic layer was separated and washed with water and brine and dried over sodium sulfate. Filtration and evaporation of the solvent gave crude product which was loaded on a Redi-Sep Gold 220 g column and eluted with 20% ethyl acetate in heptane to give the title compound. MS (ESI) m/z 337.1 (M+H)⁺.

### Example 92E

### (1r,4r)-4-(4-(((2-(trimethylsilyl)ethoxy)methoxy)methyl)pyrimidin-2-yl)cyclohexanol

To a solution of Example 92D (8.4 g) in tetrahydrofuran (100 mL) was added NaBH₄ (2.84 g). The mixture was stirred at room temperature for 3 hours. The mixture was diluted with water (20 mL) and ethyl acetate (300 mL). The organic layer was separated and washed with water and brine and dried over sodium sulfate. Filtration and evaporation of the solvent gave crude product which was loaded on a Redi-Sep Gold 120 g column and eluted with 40% ethyl acetate in heptane to give the title compound. MS (ESI) m/z 339.2 (M+H)⁺.

### Example 92F

### 2-((1r,4r)-4-(2-(allyloxy)ethoxy)cyclohexyl)-4-(((2-(trimethylsilyl)ethoxy)methoxy)methyl)pyrimidine

To a suspension of NaH (60% oil dispersion, 350 mg) in tetrahydrofuran (10 mL), a solution of Example 92E (1.3 g) in tetrahydrofuran (20 mL) was added dropwise at room temperature and the resulting suspension was stirred at room temperature for 1 hour. To the mixture, tetra-n-butylammonium iodide (760 mg) and 3-(2-bromoethoxy) prop-1-ene (1.9 g) were added. The mixture was stirred two days at 50° C. under nitrogen. The mixture was quenched with aqueous ammonium chloride, extracted with ethyl acetate (500 mL), washed with water and brine, and dried over sodium sulfate. Filtration and evaporation of the solvent gave the crude product which was loaded on a Redi-Sep Gold 120 g column and eluted with 20% ethyl acetate in heptane to give the title compound. MS (ESI) m/z 423.3 (M+H)⁺.

### Example 92G

### 3-(2-(((1r,4r)-4-(4-(((2-(trimethylsilyl)ethoxy)methoxy)methyl)pyrimidin-2-yl)cyclohexyl)oxy)ethoxy)propane-1,2-diol

To a solution of Example 92F (700 mg) in t-butanol (15 mL) and water (15 mL) at 0° C. was added AD-Mix-(3.4 g). The resulting suspension was stirred at 0° C. for 4 hours and at room temperature overnight. The mixture was quenched with sodium sulfite and extracted with ethyl acetate (3×100 mL). The combined organic phases were washed with brine and dried over sodium sulfate. Filtration and evaporation of the solvent gave the title compound. MS (ESI) m/z 457.3 (M+H)⁺.

### Example 92H

### 2-((1r,4r)-4-(2-((1,4-dioxan-2-yl)methoxy)ethoxy)cyclohexyl)-4-(((2-(trimethylsilyl)ethoxy)methoxy)methyl)pyrimidine

To a stirred solution of Example 92G (740 mg) in dichloromethane (10 mL) was added NaH (102 mg) at 0° C. The mixture was stirred for 10 minutes at 0° C. A solution of Example 91E (400 mg) in dichloromethane (5 mL) was added to the mixture and the mixture was stirred at room temperature for 3 hours. The mixture was quenched with aqueous ammonium chloride and extracted with ethyl acetate (2×200 mL). The mixture was washed with water and brine, and dried over sodium sulfate. Filtration and evaporation of the solvent gave crude product which was loaded on a Redi-Sep Gold 40 g column and eluted with 20% ethyl acetate in heptane (1 L) followed by 5% methanol in dichloromethane (500 mL) to give the title compound. MS (ESI) m/z 483.3 (M+H)⁺.

### Example 921

### (2-((1r,4r)-4-(2-((1,4-dioxan-2-yl)methoxy)ethoxy)cyclohexyl)pyrimidin-4-yl)methanol

To a solution of Example 92H (520 mg) in dichloromethane (5 mL) was added trifluoroacetic acid (5 mL). The mixture was stirred for 3 hours. The mixture was concentrated under vacuum and the residue was dissolved in dichloromethane and loaded on a Redi-Sep Gold 40 g column and eluted with 5% methanol in dichloromethane to give the title compound. MS (ESI) m/z 353.3 (M+H)⁺.

### Example 92J

### tert-butyl (7R,16R)-19,23-dichloro-10-({2-[(1r,4r)-4-{2-[(1,4-dioxan-2-yl)methoxy]ethoxy}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 92J was prepared according to the procedure for Example 57H, substituting Example 921 for Example 57G. MS (ESI) m/z 1143.5 (M+H)⁺.

### Example 92K

### (7R,16R)-19,23-dichloro-10-({2-[(1r,4r)-4-{2-[(1,4-dioxan-2-yl)methoxy]ethoxy}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 92K was prepared according to the procedure for Example 57I, substituting Example 92J for Example 57H. ¹H NMR (501 MHz, dimethylsulfoxide-d₆) δ ppm 8.67-8.61 (m, 2H), 7.36 (d, 1H), 7.17-7.09 (m, 2H), 7.07 (ddd, 2H), 6.76 (d, 1H), 6.66 (dd, 1H), 6.14 (dd, 1H), 5.75 (d, 1H), 5.05 (d, 1H), 4.98 (d, 1H), 4.80 (p, 1H), 4.37 (d, 2H), 3.63 (dt, 2H), 3.60-3.24 (m, 17H), 3.26-3.15 (m, 2H), 2.87 (dd, 1H), 2.70 (tt, 1H), 2.63 (dd, 1H), 2.58 (dd, 1H), 2.36 (s, 6H), 2.14 (s, 3H), 2.03-1.96 (m, 2H), 1.94-1.87 (m, 1H), 1.90 (s, 6H), 1.56 (dd, 1H), 1.51 (dd, 1H), 1.26-1.19 (m, 1H), 1.18 (dd, 1H). MS (ESI) m/z 1087.3 (M+H)⁺.

### Example 93

### (7R,16R)-10-{[2-(bis{2-[2-(2-methoxyethoxy)ethoxy]ethyl}amino)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 93A

### (2-(bis(2-(2-(2-methoxyethoxy)ethoxy)ethyl)amino)pyrimidin-4-yl)methanol

To a solution of (2-chloropyrimidin-4-yl)methanol (100 mg) in dioxane (4 mL) was added bis(2-(2-(2-methoxyethoxy)ethoxy)ethyl)amine (235 mg) and triethylamine (386 μL). The reaction mixture was stirred for 4 hours at 80° C. for 6 hours at 100° C. and finally for 1 hour at 110° C. in a Biotage® Initiator microwave unit. To the reaction mixture was added water and the aqueous phase was extracted twice with ethyl acetate. The combined organic extracts were washed with water and subsequently dried with sodium sulfate and filtered. The aqueous phase was again extracted twice with dichloromethane. This organic phase was combined with the other organic phase and concentrated in vacuo. Purification by chromatography on silica gel using an ISCO CombiFlash® Companion MPLC (24 g Flashpure ALOX neutral column, eluting first with 0-80% ethyl acetate in heptane and then with 0-50% methanol in dichloromethane) provided title compound. MS (APCI) m/z 418.2 (M+H)⁺.

### Example 93B

### tert butyl (7R,16R)-10-{[2-(bis{2-[2-(2-methoxyethoxy)ethoxy]ethyl}amino)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, charged with Example 16N (32.2 mg), Example 93A (20 mg), triphenylphosphine (20.9 mg) and (E)-N¹,N¹,N²,N²-tetramethyldiazene-1,2-dicarboxamide (13.7 mg) was purged for 30 minutes with argon. A mixture of toluene (0.5 mL) and tetrahydrofuran (0.5 mL) was added and the reaction mixture was stirred for 48 hours at ambient temperature and subsequently for 3 hours at 50° C. The reaction mixture was filtered to remove the formed material. To the solution was added ethyl acetate and the organic phase was washed twice with water and brine. The organic phase was concentrated in vacuo. The residue was purified by normal phase MPLC on a Teledyne-Isco-CombiFlash® system (eluting with 20-50% ethanol in ethyl acetate) to afford the title compound. MS (APCI) m/z 1208.4 (M+H)⁺.

### Example 93C

### (7R,16R)-10-{[2-(bis{2-[2-(2-methoxyethoxy)ethoxy]ethyl}amino)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 93B (27 mg) in dichloromethane (170 μL) was added trifluoroacetic acid (172 μL). The reaction mixture was stirred overnight at ambient temperature. The reaction mixture was then concentrated in vacuo. To the residue was added to cold aqueous sodium bicarbonate solution (5%) and the mixture was extracted twice with dichloromethane. The combined organic phases were dried via DryDisk® and concentrated in vacuo. The residue was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.71 (s, 1H), 8.31 (d, 1H), 7.19 (m, 2H), 7.13 (m, 2H), 6.79 (m, 1H), 6.71 (m, 2H), 6.15 (s, 1H), 5.85 (s, 1H), 4.95-4.85 (m, 3H), 4.43 (m, 2H), 3.74 (m, 4H), 3.57 (m, 5H), 3.50-3.40 (m, 16H), 3.22 (s, 6H), 2.92 (m, 1H), 2.68 (m, 2H), 2.55-2.25 (m, 8H), 2.17 (s, 3H), 1.99 (s, 3H), 1.96 (s, 3H). MS (ESI) m/z 1152.2 (M+H)⁺.

### Example 94

### (7R,16R)-19,23-dichloro-1-(4-fluorophenyl)-20,22-dimethyl-16-[(4-methylpiperazin-1-yl)methyl]-10-({2-[3-(2,5,8,11-tetraoxadodecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 94A

### (1-(4-(((tert-butyldimethylsilyl)oxy)methyl)pyrimidin-2-yl)azetidin-3-yl)methanol

Azetidin-3-ylmethanol hydrochloride (0.87 g). Example 38A (1.29 g), and triethylamine (2.79 mL) were dissolved in acetonitrile (15 mL). The reaction mixture was heated in the microwave at 80° C. for 3 hours. The reaction mixture was concentrated, and the residue dissolved in dichloromethane, and washed with water. The organic layer was dried by a PTS cartridge and concentrated to give the crude title compound. MS (ESI) m/z 310.2 (M+H)⁺.

### Example 94B

### 2-(3-(2,5,8,11-tetraoxadodecyl)azetidin-1-yl)-4-(((tert-butyldimethylsilyl)oxy)methyl)pyrimidine

Sodium hydride (500 mg, 50%) was suspended in tetrahydrofuran (2.0 mL) and Example 94A (250 mg), dissolved in tetrahydrofuran (1.5 mL), was added dropwise. The reaction mixture was stirred at room temperature for 1 hour. Tetrabutylammonium iodide (15 mg) was added. Diethyleneglycol-2-bromoethyl methyl ether (550 mg), diluted in tetrahydrofuran (1.0 mL) was added dropwise. The reaction mixture was stirred at room temperature overnight. The reaction mixture was concentrated. Purification was performed on a silica gel column (12 g, 0-20% methanol in dichloromethane). The desired fractions were combined and the solvents were removed under reduced pressure to provide the title compound. MS (ESI) m/z 456.3 (M+H)⁺.

### Example 94C

### (2-(3-(2,5,8,11-tetraoxadodecyl)azetidin-1-yl)pyrimidin-4-yl)methanol

Example 94B (342 mg) was dissolved in tetrahydrofuran (5.0 mL). Cesium fluoride (570 mg) and methanol (5.0 mL) were added. The reaction mixture was stirred at room temperature over the weekend. The reaction mixture was concentrated. The residue was washed with n-heptane and the solvent was decanted. Ethyl acetate was added to the residue and the material was filtered off. The filtrate was concentrated. Purification was performed on a silica gel column (4 g, 0-60% methanol in dichloromethane). The desired fractions were combined and the solvents were removed under reduced pressure to provide the title compound. MS (ESI) m/z 342.2 (M+H)⁺.

### Example 94D

### tert-butyl (7R,16R)-19,23-dichloro-1-(4-fluorophenyl)-20,22-dimethyl-16-[(4-methylpiperazin-1-yl)methyl]-10-({2-[3-(2,5,11-tetraoxadodecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylate

Example 94C (54 mL), Example 16N (40 mg), triphenylphosphine (52 mg), and N,N,N′,N′-tetramethylazodicarboxamide (34 mg) were combined and flushed with argon for 15 minutes. Tetrahydrofuran (1.0 mL) and toluene (1.0 mL) were mixed, flushed with argon for 15 minutes, and added to the reactants. The reaction mixture was stirred at room temperature for 1 week. The reaction mixture was concentrated. Purification was performed on a silica gel column (4 g, 0-100% ethyl acetate in n-heptane, then 100% methanol). The desired fractions were combined and the solvents were removed under reduced pressure to provide the title compound. MS (APCI) m/z 1132.4 (M+H)⁺.

### Example 94E

### (7R,16R)-19,23-dichloro-1-(4-fluorophenyl)-20,22-dimethyl-16-[(4-methylpiperazin-1-yl)methyl]-10-({2-[3-(2,5,8,11-tetraoxadodecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 94D (66 mg) was dissolved in dichloromethane (1.0 mL) and trifluoroacetic acid (470 μL) was added. The reaction mixture was stirred at room temperature for 6 hours. An aliquot analyzed by LC/MS indicated almost full conversion. The reaction mixture was concentrated at 25° C. The residue was dissolved in methanol, diluted with water, and freeze-dried. The crude material was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.70 (s, 1H), 8.29 (d, 1H), 7.21-7.18 (m, 2H), 7.14-7.11 (m, 2H), 6.79-6.76 (m, 2H), 6.71-6.69 (m, 1H), 6.14 (m, 1H), 5.84 (m, 1H), 4.97-4.86 (m, 3H), 4.46-4.39 (m, 2H), 4.07 (t, 2H), 3.75 (dd, 2H), 3.61 (d, 2H), 3.55-3.48 (m, 11H), 3.42-3.40 (m, 2H), 3.22 (s, 3H), 2.94-2.85 (m, 2H), 2.72-2.65 (m, 2H), 2.52-2.42 (m, 8H), 2.17 (s, 3H), 1.99 (s, 3H), 1.94 (s, 3H). MS (APCI) m/z 1076.3 (M+H)⁺.

### Example 95

### (7R,16R)-19,23-dichloro-1-(4-fluorophenyl)-20,22-dimethyl-16-[(4-methylpiperazin-1-yl)methyl]-10-({2-[3-(2,5,8,11,14-pentaoxapentadecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 95A

### 2-(3-(2,5,8,11,14-pentaoxapentadecyl)azetidin-1-yl)-4-(((tert-butyldimethylsilyl)oxy)methyl)pyrimidine

Sodium hydride (500 mg, 50%) was suspended in tetrahydrofuran (2.0 mL) and Example 94A (250 mg), dissolved in tetrahydrofuran (1.5 mL) was added dropwise. The reaction mixture was stirred at room temperature for 1 hour. Tetrabutylammonium iodide (15 mg) was added. 13-Bromo-2,5,8,11-tetraoxatridecane (657 mg), diluted in tetrahydrofuran (1.0 mL) was added dropwise. The reaction mixture was stirred at room temperature overnight. The reaction mixture was concentrated. Purification was performed on a silica gel column (12 g, 0-100% methanol in dichloromethane). The desired fractions were combined and the solvents were removed under reduced pressure to provide the title compound. MS (ESI) m/z 500.3 (M+H)⁺.

### Example 95B

### (2-(3-(2,5,8,11,14-pentaoxapentadecyl)azetidin-1-yl)pyrimidin-4-yl)methanol

Example 95A (371 mg) was dissolved in tetrahydrofuran (5.0 mL). Cesium fluoride (564 mg) and methanol (5.0 mL) were added. The reaction mixture was stirred at room temperature over the weekend. The reaction mixture was concentrated. The residue was washed with n-heptane and the solvent was decanted. Ethyl acetate was added to the residue and the material was filtered off. The filtrate was concentrated. Purification was performed on a silica gel column (4 g, 0-40% methanol in dichloromethane). The desired fractions were combined and the solvents were removed under reduced pressure to provide the title compound. MS (ESI) m/z 386.2 (M+H)⁺.

### Example 95C

### tert-butyl (7R,16R)-19,23-dichloro-1-(4-fluorophenyl)-20,22-dimethyl-16-[(4-methylpiperazin-1-yl)methyl]-10-({2-[3-(2,5,8,11,14-pentaoxapentadecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylate

Example 95B (63 mg), Example 16N (40 mg), triphenylphosphine (52 mg), and N,N,N′,N′-tetramethylazodicarboxamide (34 mg) were combined and flushed with argon for 15 minutes. Tetrahydrofuran (1.0 mL) and toluene (1.0 mL) were mixed, flushed with argon for 15 minutes, and added to the material. The reaction mixture was stirred at room temperature for 1 week. The reaction mixture was concentrated. Purification was performed on a silica gel column (4 g, 0-70% ethyl acetate in n-heptane, then 100% methanol). The desired fractions were combined and the solvents were removed under reduced pressure to provide the title compound. MS (APCI) m/z 1176.4 (M+H)⁺.

### Example 95D

### (7R,16R)-19,23-dichloro-1-(4-fluorophenyl)-20,22-dimethyl-16-[(4-methylpiperazin-1-yl)methyl]-10-({2-[3-(2,5,8,11,14-pentaoxapentadecan-1-yl)azetidin-1-yl]pyrimidin-4-yl}methoxy)-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 95C (63 mg) was dissolved in dichloromethane (1.0 mL) and trifluoroacetic acid (470 μL) was added. The reaction mixture was stirred at room temperature for 6 hours. The reaction mixture was concentrated at 25° C. The residue was dissolved in methanol, diluted with water, and freeze-dried. The crude material was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ 8.72 (s, 1H), 8.29 (d, 1H), 7.21-7.19 (m, 2H), 7.15-7.12 (m, 2H), 6.79 (d, 1H), 6.76 (d, 1H), 6.72 (d, 1H), 6.17 (m, 1H), 5.82 (m, 1H), 4.95 (d, 1H), 4.89-4.87 (m, 2H), 4.46-4.41 (m, 2H), 4.11-4.06 (m, 2H), 3.74 (dd, 2H), 3.61 (d, 2H), 3.51-3.49 (m, 16H), 3.42-3.40 (m, 1H), 3.22 (s, 3H), 2.94-2.85 (m, 2H), 2.71-2.64 (m, 2H), 2.52-2.42 (m, 8H), 2.18 (s, 3H), 1.97 (s, 3H), 1.96 (s, 3H). MS (APCI) m/z 1020.4 (M+H)⁺.

### Example 96

### (7R,16R)-19,23-dichloro-10-({2-[(1s,4s)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 96A

### (2-((1s,4s)-4-fluoro-4-((2-(2-methoxyethoxy)ethoxy)methyl)cyclohexyl)pyrimidin-4-yl)methanol

The title compound was prepared as described in Example 105A, replacing Example 101I with Example 86E.

### Example 96B

### tert-butyl (7R,16R)-19,23-dichloro-10-({2-[(1s,4s)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 96A. MS (ESI) m/z 1135.6 (M+H)⁺.

### Example 96C

### (7R,16R)-19,23-dichloro-10-({2-[(1s,4s)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M, replacing Example 101L with Example 96B. ¹H NMR (500 MHz, dimethylsulfoxide-d₆) δ ppm 8.75-8.70 (m, 2H), 7.44 (d, 1H), 7.23-7.17 (m, 2H), 7.14 (ddd, 2H), 6.85 (d, 1H), 6.74 (dd, 1H), 6.21 (dd, 1H), 5.81 (d, 1H), 5.14 (d, 1H), 5.07 (d, 1H), 4.87 (p, 1H), 4.44 (d, 2H), 3.64-3.57 (m, 4H), 3.54 (ddd, 5H), 3.50 (s, 2H), 3.48-3.42 (m, 4H), 3.25 (s, 3H), 2.98-2.91 (m, 1H), 2.88-2.81 (m, 1H), 2.74-2.63 (m, 3H), 2.54 (s, 1H), 2.45 (s, 2H), 2.40 (s, 2H), 2.20 (s, 3H), 2.00-1.89 (m, 8H), 1.84 (tt, 4H), 1.66-1.56 (m, 1H), 1.56-1.48 (m, 1H). MS (ESI) m/z 1075.0 (M−H)⁻.

### Example 97

### (7R,16R)-19,23-dichloro-10-({2-[(4S*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 97A

### 8-fluoro-8-(2,5,8,11,14,17-hexaoxaoctadecyl)-1,4-dioxaspiro[4.5]decane

The title compound was prepared as described in Example 101E, replacing 2-(2-(2-methoxyethoxy)ethoxy)ethyl 4-methylbenzenesulfonate with 2,5,8,11,14-pentaoxahexadecan-16-yl 4-methylbenzenesulfonate.

### Example 97B

### 4-fluoro-4-(2,5,8,11,14,17-hexaoxaoctadecyl)cyclohexanone

The title compound was prepared as described in Example 101F, replacing Example 101E with Example 97A.

### Example 97C

### 4-fluoro-4-(2,5,8,11,14,17-hexaoxaoctadecyl)cyclohex-1-en-1-yl trifluoromethanesulfonate

The title compound was prepared as described in Example 101G, replacing Example 101F with Example 97B.

### Example 97D

### 2-(4-fluoro-4-(2,5,8,11,14,17-hexaoxaoctadecyl)cyclohex-1-en-1-yl)-4,4,5,5-tetramethyl-1,3,2-dioxaborolane

The title compound was prepared as described in Example 101H, replacing Example 101G with Example 97C.

### Example 97E

### (2-(4-fluoro-4-(2,5,8,11,14,17-hexaoxaoctadecyl)cyclohex-1-en-1-yl)pyrimidin-4-yl)methanol

The title compound was prepared as described in Example 101I, replacing Example 101H with Example 97D.

### Example 97F

### (S)-(2-(4-fluoro-4-(2,5,8,11,14,17-hexaoxaoctadecyl)cyclohex-1-en-1-yl)pyrimidin-4-yl)methanol

The title compound was prepared as described in Example 101J, replacing Example 101I with Example 97E. ¹H NMR (400 MHz, CDCl₃) δ ppm 8.62 (d, 1H), 7.20 (br s, 1H), 7.09 (d, 1H), 4.72 (s, 2H), 3.84-3.48 (m, 22H), 3.36 (s, 3H), 2.77 (br s, 2H), 2.66-2.44 (m, 2H), 2.19-2.06 (m, 1H), 2.00-1.79 (m, 1H)

### Example 97G

### (R)-(2-(4-fluoro-4-(2,5,8,11,14,17-hexaoxaoctadecyl)cyclohex-1-en-1-yl)pyrimidin-4-yl)methanol

The title compound was prepared as described in Example 101J, replacing Example 101I with Example 97E. ¹H NMR (400 MHz, CDCl₃) δ ppm 8.63 (d, 1H), 7.22 (br s, 1H), 7.08 (d, 1H), 4.73 (s, 2H), 3.80-3.50 (m, 22H), 3.38 (s, 3H), 2.78 (br s, 2H), 2.67-2.47 (m, 2H), 2.19-2.08 (m, 1H), 2.01-1.81 (m, 1H).

### Example 97H

### tert-butyl (7R,16R)-19,23-dichloro-10-({2-[(4S*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L, replacing Example 101J with Example 97F. MS (ESI) m/z 633.8 (M+H)²⁺.

### Example 97I

### (7R,16R)-19,23-dichloro-10-({2-[(4S*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M, replacing Example 101L with Example 97H. ¹H NMR (500 MHz, dimethylsulfoxide-d₆) δ ppm 8.76-8.71 (m, 2H), 7.41 (d, 1H), 7.20 (dd, 2H), 7.14 (td, 3H), 6.83 (d, 1H), 6.73 (dd, 1H), 6.20 (dd, 1H), 5.82 (d, 1H), 5.16 (d, 1H), 5.08 (d, 1H), 4.87 (p, 1H), 4.44 (d, 2H), 3.64-3.58 (m, 4H), 3.58-3.48 (m, 18H), 3.43-3.40 (m, 2H), 3.22 (s, 3H), 2.95 (dd, 1H), 2.76-2.64 (m, 4H), 2.47-2.30 (m, 9H), 2.19 (s, 3H), 2.03 (q, 1H), 1.97 (d, 6H), 1.87-1.69 (m, 1H). MS (ESI) m/z 1207.4 (M+H)⁺.

### Example 98

### (7R,16R)-19,23-dichloro-10-({2-[(1r,4r)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 98A

### (2-((1r,4r)-4-fluoro-4-((2-(2-methoxyethoxy)ethoxy)methyl)cyclohexyl)pyrimidin-4-yl)methanol

The title compound was also isolated from the preparation of Example 96A.

### Example 98B

### tert-butyl (7R,16R)-19,23-dichloro-10-({2-[(1r,4r)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 98A. MS (ESI) m/z 1135.6 (M+H)⁺.

### Example 98C

### (7R,16R)-19,23-dichloro-10-({2-[(1r,4r)-4-fluoro-4-{[2-(2-methoxyethoxy)ethoxy]methyl}cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M, replacing Example 101L with Example 98B. ¹H NMR (500 MHz, dimethylsulfoxide-d₆) δ ppm 8.72 (d, 2H), 7.45 (d, 1H), 7.24-7.17 (m, 2H), 7.14 (ddd, 2H), 6.84 (d, 1H), 6.73 (dd, 1H), 6.19 (dd, 1H), 5.83 (d, 1H), 5.13 (d, 1H), 5.06 (d, 1H), 4.88 (p, 1H), 4.48-4.38 (m, 2H), 3.62-3.49 (m, 9H), 3.43-3.39 (m, 2H), 3.21 (s, 3H), 2.99 (dq, 1H), 2.94 (dd, 1H), 2.74-2.63 (m, 2H), 2.46-2.33 (m, 8H), 2.20 (s, 3H), 1.97 (d, 7H), 1.94 (s, 2H), 1.87-1.81 (m, 1H), 1.79 (d, 1H), 1.66 (qd, 2H).

### Example 99

### (7R,16R)-19,23-dichloro-1-(4-fluorophenyl)-10-[(6-{2-[2-(2-methoxyethoxy)ethoxy]ethoxy}pyridin-2-yl)methoxy]-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 99A

### tert-butyl (4R,9R)-13,15-dichloro-26-(4-fluorophenyl)-66-((6-(2-(2-(2-methoxyethoxy)ethoxy)ethoxy)pyridin-2-yl)methoxy)-12,16-dimethyl-9-((4-methylpiperazin-1-yl)methyl)-3,7,10-trioxa-2(5,4)-thieno[2,3-d]pyrimidina-1(1,4),6(1,3)-dibenzenacyclodecaphane-4-carboxylate

Example 16N (25 mg), (6-(2-[2-(2-methoxyethoxy)ethoxy]ethoxy)pyridin-2-yl)methanol (29 mg), triphenylphosphine (37 mg), and N,N,N′,N′-tetramethylazodicarboxamide (24 mg) were combined under argon atmosphere. Tetrahydrofuran (0.6 mL) and toluene (0.6 mL) were added. The reaction mixture was stirred at room temperature over the weekend. All volatiles were removed and the residue was partitioned between dichloromethane and aqueous saturated sodium bicarbonate solution. The aqueous layer was extracted with dichloromethane twice. The combined organic extracts were dried over magnesium sulfate, filtrated, and concentrated. The residue was purified on silica gel column (4 g, 0-8% methanol in dichloromethane). The desired fractions were combined and the solvents were removed under reduced pressure to provide the title compound. MS (ESI) m/z 885.3 (M+H)⁺.

### Example 99B

### (4R,9R)-13,15-dichloro-26-(4-fluorophenyl)-66-((6-(2-(2-(2-methoxyethoxy)ethoxy)ethoxy)pyridin-2-yl)methoxy)-12,16-dimethyl-9-((4-methylpiperazin-1-yl)methyl)-3,7,10-trioxa-2(5,4)-thieno[2,3-d]pyrimidina-1(1,4),6(1,3)-dibenzenacyclodecaphane-4-carboxylic Acid

Example 99A (27 mg) was dissolved in dichloromethane (1.0 mL). Trifluoroacetic acid (200 μL) was added and the reaction mixture was stirred at room temperature overnight. All volatiles were removed at room temperature. The residue was dissolved in dichloromethane and concentrated at room temperature again. The obtained residue was dissolved in methanol (0.5-1.0 mL), diluted with water (8 mL) and the solution was concentrated once more at room temperature. The remaining aqueous solution was freeze-dried. The crude material was purified by HPLC (Phenomenex® Gemini NX C18 21×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ 8.70 (s, 1H), 7.69 (dd, 1H), 7.20 (t, 2H), 7.14-7.09 (m, 3H), 6.83 (d, 1H), 6.75 (d, 1H), 6.71 (m, 1H), 6.14 (m, 1H), 5.85 (m, 1H), 5.03 (d, 1H), 4.97 (d, 1H), 4.91 (m, 1H), 4.46-4.40 (m, 2H), 4.38-4.36 (m, 2H), 3.73-3.72 (m, 2H), 3.57-3.55 (m, 3H), 3.53-3.49 (m, 5H), 3.42-3.40 (m, 3H), 3.22 (s, 3H), 2.91 (d, 1H), 2.68 (qd, 2H), 2.54-2.28 (m, 8H), 2.17 (s, 3H), 1.98 (m, 3H), 1.94 (m, 3H). MS (ESI) m/z 1006.1 (M+H)⁺.

### Example 100

### (7R,16R)-19,23-dichloro-10-({2-[(4R*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 100A

### tert-butyl (7R,16R)-19,23-dichloro-10-({2-[(4R*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L, replacing Example 101J with Example 97G. MS (ESI) m/z 633.7 (M+H)²⁺.

### Example 100B

### (7R,16R)-19,23-dichloro-10-({2-[(4R*)-4-fluoro-4-(2,5,8,11,14,17-hexaoxaoctadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M, replacing Example 101L with Example 100A. ¹H NMR (500 MHz, dimethylsulfoxide-d₆) δ ppm 8.83-8.60 (m, 2H), 7.42 (d, 1H), 7.23-7.17 (m, 2H), 7.13 (td, 3H), 6.83 (d, 1H), 6.73 (dd, 1H), 6.20 (dd, 1H), 5.82 (d, 1H), 5.21-5.04 (m, 2H), 4.87 (p, 1H), 4.52-4.34 (m, 2H), 3.64-3.48 (m, 22H), 3.41 (dd, 3H), 3.22 (s, 3H), 2.95 (dd, 1H), 2.78-2.59 (m, 3H), 2.47-2.27 (m, 9H), 2.19 (s, 3H), 2.09-1.92 (m, 7H), 1.86-1.70 (m, 1H). MS (ESI) m=1207.6 (M+H)⁺.

### Example 101

### (7R,16R)-19,23-dichloro-1-(4-fluorophenyl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 101A

### 8-methylene-1,4-dioxaspiro[4.5]decane Commented [A14]: Warning

To a solution of methyltriphenylphosphonium bromide (68.6 g) in tetrahydrofuran (200 mL) was added n-butyllithium (77 mL, 2.5 M in tetrahydrofuran) at −78° C. The reaction mixture was stirred for 10 minutes at −78° C., then 30 minutes at 0° C., then cooled to −78° C. A solution of 1,4-dioxaspiro[4.5]decan-8-one (50 g) in tetrahydrofuran (200 mL) was added. The reaction mixture was stirred for 16 hours at 25° C. and was filtered. The filtrate was concentrated. The residue was purified by column chromatography on silica gel (eluted with petroleum ether:ethyl acetate=5:1) to provide the title compound. ¹H NMR (400 MHz, CDCl₃) δ ppm 4.67 (s, 2H), 3.97 (s, 4H), 2.31-2.27 (m, 4H), 1.72-1.64 (m, 3H).

### Example 101B

### 8-(bromomethyl)-8-fluoro-1,4-dioxaspiro[4.5]decane

To a mixture of Example 101A (10 g, 64.8 mmol) and 1-bromopyrrolidine-2,5-dione (13.85 g) in dichloromethane (150 mL) was added triethylamine trihydrofluoride (15.68 g) at 0° C. The reaction mixture was stirred at 20° C. for 2 hours, poured into saturated aqueous NaHCO₃ solution (500 mL) and extracted with dichloromethane (500 mL). The combined extracts were washed with 0.1M aqueous HCl (2×200 mL) and 5% sodium hydrogen carbonate solution (2×200 mL), dried over magnesium sulfate, filtered, and concentrated. The residue was purified by column chromatography on silica gel (eluted with petroleum ether:ethyl acetate=3:1) to provide the title compound. ¹H NMR (400 MHz, CDCl₃) δ ppm 3.99-3.92 (m, 4H), 3.48 (d, J=18 Hz, 2H), 2.10-2.05 (m, 2H), 1.91-1.64 (m, 6H).

### Example 101C

### (8-fluoro-1,4-dioxaspiro[4.5]decan-8-yl)methyl acetate

To a mixture of Example 101B (10 g), potassium iodide (0.656 g) in N,N-dimethylformamide (100 mL) was added potassium acetate (38.8 g) at 25° C. The mixture was heated at 135° C. for 16 hours, cooled, poured into water and extracted with ethyl acetate. The combined organic layer was washed with brine (2×100 mL). The organic phase was dried over sodium sulfate, filtered, and concentrated. The residue was purified by column chromatography on silica gel (eluted with petroleum ether:ethyl acetate=3:1 to 1:1) to provide the title compound. ¹H NMR (400 MHz, CDCl₃) δ ppm 4.11 (d, 2H), 3.99-3.93 (m, 4H), 2.10 (s, 3H), 1.97-1.63 (m, 8H).

### Example 101D

### (8-fluoro-1,4-dioxaspiro[4.5]decan-8-yl)methanol

To a solution of Example 101C (25 g) in tetrahydrofuran (200 mL) and water (100 mL) was added lithium hydroxide monohydrate (6.78 g) at 0° C. The reaction mixture was stirred for 16 hours at 25° C. poured into water (500 mL) and extracted with ethyl acetate (3×500 mL). The combined organic phase was washed with brine (2×100 mL). The organic layers were combined, dried over magnesium sulfate, filtered, and concentrated. The residue was purified by column chromatography on silica gel (eluted with petroleum ether:ethyl acetate=3:1) to provide the title compound. ¹H NMR (400 MHz, CDCl₃) δ ppm 3.99-3.93 (m, 4H), 3.64-3.57 (m, 2H), 2.03-2.01 (m, 2H), 1.89-1.86 (m, 3H), 1.68-1.63 (m, 4H).

### Example 101E

### 8-(2,5,8,11-tetraoxadodecyl)-8-fluoro-1,4-dioxaspiro[4.5]decane

To a solution of Example 101D (3.5 g) in tetrahydrofuran (100 mL) was added NaH (1.472 g) at 0° C. The mixture was stirred for 10 minutes and a solution of 2-(2-(2-methoxyethoxy)ethoxy)ethyl 4-methylbenzenesulfonate (5.86 g) in tetrahydrofuran (100 mL) was added. The reaction was stirred at 50° C. for 12 hours, poured into ice water (200 mL) and exacted with ethyl acetate (2×300 mL). The organic phases were combined and washed with brine (100 mL), dried over magnesium sulfate, filtered, and concentrated. The residue was purified by column chromatography on silica gel (eluting with petroleum ether:ethyl acetate=3:1 to 1:1) to provide the title compound. ¹H NMR (400 MHz, CDCl₃) δ ppm 4.01-3.88 (m, 4H), 3.71-3.61 (m, 10H), 3.57-3.51 (m, 3H), 3.48 (s, 1H), 3.37 (s, 3H), 2.04-1.81 (m, 4H), 1.81-1.55 (m, 4H).

### Example 101F

### 4-(2,5,8,11-tetraoxadodecyl)-4-fluorocyclohexanone

To a solution of Example 101E (3.3 g) in tetrahydrofuran (50 mL) was added aqueous HCl (50 mL, 6 M) at 0° C. The reaction mixture was stirred at 25° C. for 16 hours and cooled to 0° C. Solid NaOH was added to adjust pH value to 8. The mixture was extracted with ethyl acetate (8×100 mL). The combined organic layers were dried over sodium sulfate, filtered, and concentrated to provide the title compound. ¹H NMR (400 MHz, CDCl₃) δ ppm 3.73-3.60 (m, 11H), 3.57 (s, 1H), 3.55-3.51 (m, 2H), 3.36 (s, 3H), 2.66 (dt, 2H), 2.38-2.22 (m, 4H), 2.01-1.76 (m, 2H).

### Example 101G

### 4-(2,5,8,11-tetraoxadodecyl)-4-fluorocyclohex-1-en-1-yl trifluoromethanesulfonate

To a stirred solution of diisopropylamine (1.35 g) in dry tetrahydrofuran (30 mL) was added n-butyllithium (5.34 mL) under nitrogen at 0° C. The mixture was stirred for 30 minutes and a solution of Example 101F (2.6 g) in dry tetrahydrofuran (30 mL) was added. The mixture was stirred for 15 minutes at −78° C. and a solution of 1,1,1-trifluoro-N-phenyl-N-((trifluoromethyl) sulfonyl) methanesulfonamide (4.13 g) in tetrahydrofuran (30 mL) was added. The reaction was warmed to 20° C. stirred for 16 hours, poured into ice water (200 mL) and exacted with ethyl acetate (150 mL). The organic phases were combined, washed with brine (100 mL), dried over magnesium sulfate, filtered, and concentrated. The residue was purified by column chromatography on silica gel (eluting with petroleum ether:ethyl acetate=3:1 to 1:1) to provide the title compound. ¹H NMR (400 MHz, CDCl₃) δ ppm 5.59 (br s, 1H), 3.66-3.55 (m, 10H), 3.53 (s, 1H), 3.50-3.45 (m, 3H), 3.30 (s, 3H), 2.54 (ddt, 1H), 2.42 (br s, 1H), 2.39-2.21 (m, 2H), 2.11-1.98 (m, 1H), 1.93-1.71 (m, 1H).

### Example 101H

### 2-(4-(2,5,8,11-tetraoxadodecyl)-4-fluorocyclohex-1-en-1-yl)-4,4,5,5-tetramethyl-1,3,2-dioxaborolane

To a solution of Example 101G (3.5 g) in 1,4-dioxane (100 mL) were added 4,4,4′,4′,5,5,5′,5′-octamethyl-2,2′-bi(1,3,2-dioxaborolane) (2.72 g), potassium acetate (1.619 g) and [1,1-bis(diphenylphosphino)ferrocene]palladium (II) chloride (0.673 g) at 20° C. under nitrogen. The mixture was stirred at 80° C. for 12 hours under nitrogen atmosphere, cooled to 20° C. and filtered. The filtrate was concentrated and the residue was purified by column chromatography on silica gel (eluting with petroleum ether:ethyl acetate=100:1 to 10:1) to provide the title compound. ¹H NMR (400 MHz, CDCl₃) δ ppm 6.43 (br d, 1H), 3.72-3.62 (m, 10H), 3.58-3.53 (m, 3H), 3.51 (s, 1H), 3.38 (s, 3H), 2.45-2.13 (m, 4H), 1.94-1.83 (m, 1H), 1.82-1.62 (m, 1H), 1.32-1.19 (m, 27H).

### Example 101I

### (2-(4-(2,5,8,11-tetraoxadodecyl)-4-fluorocyclohex-1-en-1-yl)pyrimidin-4-yl)methanol

To a solution of Example 101H (3.12 g) and (2-chloropyrimidin-4-yl)methanol (0.7 g) in dioxane (40 mL) was added tetrakis(triphenylphosphine)-palladium (0) (0.28 g) and saturated aqueous NaHCO₃ solution (20 mL). The mixture was heated at 110° C. for 16 hours under nitrogen atmosphere, cooled to 15° C. and exacted with ethyl acetate (3×50 mL). The combined organic phases were washed with brine (2×30 mL), dried over magnesium sulfate, filtered, and concentrated. The residue was purified by HPLC on a Shimadzu LC-8A preparative HPLC (Column: Phenomenex Luna™ (2) C18 250×50 10 μm: Mobile phase: A for H₂O (0.090% trifluoroacetic acid) and B for acetonitrile; Gradient: B from 15% to 35% in 20 min; Flow rate: 60 mL/minute; Wavelength: 220 and 254 nm) to provide the title compound. MS (ESI) m/z 385.3 (M+H)⁺.

### Example 101J

### (S)(2-(4-(2,5,8,11-tetraoxadodecyl)-4-fluorocyclohex-1-en-1-yl)pyrimidin-4-yl)methanol

Example 101I (0.6 g) was separated on a Thar SFC 80 preparative SFC (Column: Chiralpak AD-H 250*30 mm i.d. 5 μm; Mobile phase: A for CO₂ and B for ethanol (0.1% ammonium hydroxide); Gradient: B %=35%; Flow rate: 62 g/minute: Wavelength: 220 nm; Column temperature: 40° C.: System back pressure: 100 bar) to provide enatiomerically pure title compound. The stereochemistry was 35 arbitrarily assigned. ¹H NMR (400 MHz, CDCl₃) δ ppm 8.63 (d, 1H), 7.23 (br s, 1H), 7.07 (d, 1H), 4.73 (s, 2H), 3.84-3.50 (m, 15H), 3.38 (s, 3H), 2.87-2.73 (m, 2H), 2.71-2.47 (m, 2H), 2.21-2.08 (m, 1H), 2.01-1.81 (m, 1H). MS (ESI) m=385.3 (M+H)⁺.

### Example 101K

### (R)-(2-(4-(2,5,8,11-tetraoxadodecyl)-4-fluorocyclohex-1-en-1-yl)pyrimidin-4-yl)methanol

The title compound was also obtained during the preparation for Example 101J. The stereochemistry was arbitrarily assigned. ¹H NMR (400 MHz, CDCl₃) δ ppm 8.63 (d, 1H), 7.22 (br s, 1H), 7.07 (d, 1H), 4.73 (s, 2H), 3.77-3.52 (m, 15H), 3.38 (s, 3H), 2.79 (br dd, 2H), 2.67-2.46 (m, 2H), 2.21-2.08 (m, 1H), 2.01-1.79 (m, 1H). MS (ESI) m/z 385.3 (M+H)⁺.

### Example 101L

### tert-butyl (7R,16R)-19,23-dichloro-1-(4-fluorophenyl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 16N (30 mg), Example 101J (21.4 mg) and Ph₃P (38.9 mg) in tetrahydrofuran (1 mL) and toluene (1 mL) was added (E)-N¹,N¹,N²,N²-tetramethyldiazene-1,2-dicarboxamide (25.5 mg). The reaction mixture was heated at 60° C. overnight, diluted with dichloromethane and purified by flash chromatography on a Teledyne Isco CombiFlash® system, eluting with 0-10% methanol in dichloromethane to provide the title compound. MS (ESI) m/z 1175.5 (M+H)⁺.

### Example 101M

### (7R,16R)-19,23-dichloro-1-(4-fluorophenyl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 101L (50 mg) in dichloromethane (5 mL) was treated with trifluoroacetic acid (2.5 mL) overnight and the mixture was concentrated. The residue was co-concentrated with methanol (5 mL) three times and dissolved in methanol. The solution was cooled in an ice bath, mixed with 1.5 mL of trimethylamine and concentrated. The residue was dissolved in dimethylsulfoxide (2 mL), methanol (2 mL) and saturated ammonium acetate (1 mL) and purified by reverse phase HPLC on a ACCQPrep HP125 system, eluting with 40-65% acetonitrile in 5 mM ammonium acetate water solution to provide the title compound. ¹H NMR (500 MHz, dimethylsulfoxide-d₆) δ ppm 8.69-8.64 (m, 2H), 7.35 (d, 1H), 7.13 (t, 2H), 7.07 (td, 3H), 6.76 (d, 1H), 6.66 (dd, 1H), 6.13 (dd, 1H), 5.75 (d, 1H), 5.09 (d, 1H), 5.01 (d, 1H), 4.84-4.76 (m, 1H), 4.41-4.34 (m, 2H), 3.58-3.42 (m, 14H), 3.35 (dd, 3H), 3.16 (s, 3H), 2.88 (dd, 1H), 2.70-2.54 (m, 4H), 2.40-2.22 (m, 8H), 2.12 (s, 3H), 2.00-1.93 (m, 1H), 1.90 (d, 6H), 1.77-1.64 (m, 1H). MS (ESI) m/z 1119.4 (M+H)⁺.

### Example 102

### (7R,16R)-19,23-dichloro-10-[(2-{1-[(2-{[(2R)-1,4-dioxan-2-yl]methoxy}ethoxy)methyl]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 102A

### 2-(1-((2-((tert-butyldimethylsilyl)oxy)ethoxy)methyl)cyclohexyl)-4-(dimethoxymethyl)pyrimidine

To a stirring solution of Example 88C (600 mg) and (2-bromoethoxy)-tert-butyldimethylsilane (1078 mg) in acetonitrile (18 mL) was slowly added sodium hydride (108 mg) and the mixture was stirred at 45° C. for 16 hours. Saturated aqueous ammonium chloride was added. The mixture was extracted twice with ethyl acetate and the organic layer was washed with brine, dried over anhydrous sodium sulfate, filtered and concentrated. The crude product was purified by silica gel flash chromatography on AnaLogix IntelliFlash²⁸⁰ system eluting with 0-40% ethyl acetate in heptanes to give the title compound. MS (ESI) m/z 425.4 (M+H)⁺.

### Example 102B

### 2-((1-(4-(dimethoxymethyl)pyrimidin-2-yl)cyclohexyl)methoxy)ethanol

To a solution of Example 102A (420 mg) in tetrahydrofuran (3.0 mL) was added tetrabutylammonium fluoride (1M in tetrahydrofuran, 1.978 mL). The mixture was stirred for 40 minutes. The mixture was concentrated and the crude product was purified by silica gel flash chromatography on AnaLogix IntelliFlash²⁸⁰ system (solvent A=3:1 ethyl acetate:ethanol, solvent B=heptanes; eluting with 15-60% A to B) to give the title compound. MS (ESI) m/z 311.1 (M+H)⁺.

### Example 102C

### (R)-2-(1-((2-((1,4-dioxan-2-yl)methoxy)ethoxy)methyl)cyclohexyl)-4-(dimethoxymethyl)pyrimidine

To a stirring solution of Example 102B (200 mg) and Example 72D (253 mg) in acetonitrile (6 mL) was added sodium hydride (30.9 mg) in one portion and the mixture was stirred at 45° C. for 1 day. A few drops of saturated aqueous ammonium chloride solution were added. The mixture was concentrated onto silica gel and purified by flash chromatography on AnaLogix IntelliFlash²⁸⁰ system (solvent A=3:1 ethyl acetate:ethanol; solvent B=heptanes, eluting with 30-100% A to B)) to give the title compound. LC/MS (ESI) m/z 411.24 (M+H)⁺.

### Example 102D

### (R)-2-(1-((2-((1,4-dioxan-2-yl)methoxy)ethoxy)methyl)cyclohexyl)pyrimidine-4-carbaldehyde

The title compound was prepared by substituting Example 102C for Example 88D in Example 88E. LC/MS (ESI) m/z 365.22 (M+H)⁺.

### Example 102E

### (R)-(2-(1-((2-((1,4-dioxan-2-yl)methoxy)ethoxy)methyl)cyclohexyl)pyrimidin-4-yl)methanol

The title compound was prepared by substituting Example 102D for Example 88E in Example 88F. MS (ESI) m/z 367.3 (M+H)⁺.

### Example 102F

### tert-butyl (7R,16R)-19,23-dichloro-10-[(2-{1-[(2-{[(2R)-1,4-dioxan-2-yl]methoxy}ethoxy)methyl]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 102E for Example 88F in Example 88G. MS (ESI) m/z 1157.9 (M+H)⁺.

### Example 102G

### (7R,16R)-19,23-dichloro-10-[(2-{1-[(2-{[(2R)-1,4-dioxan-2-yl]methoxy}ethoxy)methyl]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 102F for Example 51E in Example 51F. ¹H NMR (501 MHz, dimethylsulfoxide-d₆) δ ppm 8.76 (d, 1H), 8.73 (s, 1H), 7.42 (d, 1H), 7.24-7.07 (m, 4H), 6.87 (d, 1H), 6.73 (dd, 1H), 6.24 (dd, 1H), 5.81 (d, 1H), 5.18-4.99 (m, 2H), 4.91-4.82 (m, 1H), 4.44 (d, 2H), 2.22 (s, 3H), 3.70-2.26 (m, 29H), 1.97 (s, 3H), 1.96 (s, 3H), 1.60-1.36 (m, 5H), 1.32-1.19 (m, 3H). MS (ESI) m/z 1101.6 (M+H)⁺.

### Example 103

### (7R,16R)-19,23-dichloro-10-({2-[(2S)-2-({2-[(1,4-dioxan-2-yl)methoxy]ethoxy}methyl) morpholin-4-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 103A

### (S)-(4-(4-(((tert-butyldimethylsilyl)oxy)methyl)pyrimidin-2-yl) morpholin-2-yl)methanol

A solution of (S)-morpholin-2-ylmethanol, trifluoroacetic acid salt (420 mg), Example 38A (390 mg) and N,N-diisopropylethylamine (1.6 mL) in acetonitrile (3.8 mL) was heated to 80° C. overnight. The reaction was cooled, diluted with water and extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 0-45% ethyl acetate in dichloromethane to give the title compound.

### Example 103B

### (S)-2-((2-(allyloxy)ethoxy)methyl)-4-(4-(((tert-butyldimethylsilyl)oxy)methyl)pyrimidin-2-yl)morpholine

To a solution of Example 103A (420 mg) in tetrahydrofuran (8.2 mL) at 0° C. was added sodium hydride (110 mg, 60% oil dispersion), and the reaction was allowed to stir for 1 hour as it warmed to room temperature. Tetrabutylammonium iodide (460 mg) and 3-(2-bromoethoxy) prop-1-ene (670 mg) were added, and the reaction was allowed to stir at room temperature overnight. The reaction was quenched with saturated aqueous ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 0-35% ethyl acetate in dichloromethane to give the title compound. ¹H NMR (500 MHz, dimethylsulfoxide-d₆) δ ppm 8.38 (d, 1H), 6.70 (d, 1H), 5.93-5.82 (m, 1H), 5.30-5.21 (m, 1H), 5.17-5.09 (m, 1H), 4.55 (s, 2H), 4.53-4.47 (m, 1H), 4.41-4.33 (m, 1H), 4.00-3.93 (m, 2H), 3.92-3.87 (m, 1H), 3.62-3.42 (m, 8H), 2.98-2.86 (m, 1H), 2.76-2.65 (m, 1H), 0.91 (s, 9H), 0.08 (s, 6H).

### Example 103C

### 3-(2-(((S)-4-(4-(((tert-butyldimethylsilyl)oxy)methyl)pyrimidin-2-yl) morpholin-2-yl)methoxy)ethoxy)propane-1,2-diol

To a solution of Example 103B (300 mg) in 1-butanol (3.5 mL) and water (3.5 mL) at 0° C. was added AD-Mix alpha (1.5 g), and the reaction was stirred for 4 hours at 0° C. The reaction was warmed to room temperature and stirred overnight. The reaction was quenched with solid sodium sulfite, diluted with water and extracted with ethyl acetate three times. The combined organic layers were washed with brine, dried over anhydrous sodium sulfate, filtered and concentrated. The crude product was resubmitted to the same conditions and workup procedure to give the title compound that was used in the next step without further purification.

### Example 103D

### (2S)-2-((2-((1,4-dioxan-2-yl)methoxy)ethoxy)methyl)-4-(4-(((tert-butyldimethylsilyl)oxy)methyl)pyrimidin-2-yl)morpholine

To a solution of Example 103C (320 mg) in dichloromethane (4.7 mL) at room temperature was added sodium hydride (51 mg, 60% oil dispersion), and the reaction was allowed to stir for 10 minutes. A solution of Example 91E (300 mg) in dichloromethane (2.4 mL) was added, and the reaction was allowed to stir for 5 hours. The reaction was quenched with saturated aqueous ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+12 g gold silica gel column eluting with 0-55% ethyl acetate in dichloromethane to give the title compound. ¹H NMR (500 MHz, dimethylsulfoxide-d₆) δ ppm 8.38 (d, 1H), 6.71 (d, 1H), 4.56 (s, 2H), 4.53-4.46 (m, 1H), 4.43-4.33 (m, 1H), 3.95-3.87 (m, 1H), 3.75-3.34 (m, 16H), 3.29-3.20 (m, 1H), 2.98-2.87 (m, 1H), 2.76-2.66 (m, 1H), 0.91 (s, 9H), 0.09 (s, 6H).

### Example 103E

### (2-((2S)-2-((2-((1,4-dioxan-2-yl)methoxy)ethoxy)methyl) morpholino)pyrimidin-4-yl)methanol

To a solution of Example 103D (90 mg) in tetrahydrofuran (630 μL) and methanol (320 μL) was added cesium fluoride (140 mg), and the reaction was allowed to stir for 5 hours. The reaction was concentrated, and the residue was taken up in ethyl acetate with sonication, filtered over diatomaceous earth and concentrated. The residue was purified by normal phase MPLC on a Teledyne Isco CombiFlash® Rf+4 g gold silica gel column, eluting with 20-100% ethyl acetate in dichloromethane to give the title compound. ¹H NMR (500 MHz, dimethylsulfoxide-d₆) δ ppm 8.35 (d, 1H), 6.76 (d, 1H), 5.46-5.38 (m, 1H), 4.55-4.45 (m, 1H), 4.43-4.32 (m, 3H), 3.95-3.86 (m, 1H), 3.75-3.35 (m, 16H), 3.30-3.20 (m, 1H), 2.97-2.85 (m, 1H), 2.75-2.63 (m, 1H).

### Example 103F

### tert-butyl (7R,16R)-19,23-dichloro-10-({2-[(25)-2-({2-[(1,4-dioxan-2-yl)methoxy]ethoxy}methyl) morpholin-4-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A vial containing Example 103E (51 mg), Example 16N (37 mg), triphenylphosphine (36 mg) and N,N,N′,N′-tetramethylazodicarboxamide (24 mg) in toluene (110 μL) and tetrahydrofuran (110 μL) was allowed to stir at 50° C. for 4 hours. The reaction was cooled, diluted with ethyl acetate, filtered over diatomaceous earth and concentrated. The residue was purified by normal phase MPLC on a Teledyne Isco CombiFlash® Rf+4 g gold silica gel column eluting with 0.5-9% methanol in dichloromethane to give the title compound. ¹H NMR (500 MHz, dimethylsulfoxide-d₆) δ ppm 8.74 (s, 1H), 8.39 (d, 1H), 7.27-7.13 (m, 5H), 6.90-6.75 (m, 3H), 6.08-5.99 (m, 1H), 5.70-5.62 (m, 1H), 5.04-4.85 (m, 2H), 4.80-4.69 (m, 1H), 4.57-4.35 (m, 4H), 3.97-3.88 (m, 1H), 3.74-3.34 (m, 14H), 3.29-3.21 (m, 1H), 3.02-2.90 (m, 1H), 2.89-2.82 (m, 1H), 2.78-2.58 (m, 3H), 2.43-2.21 (m, 4H), 2.14 (s, 3H), 2.10 (s, 3H), 1.89 (s, 3H), 1.06 (s, 9H).

### Example 103G

### (7R,16R)-19,23-dichloro-10-({2-[(2S)-2-({2-[(1,4-dioxan-2-yl)methoxy]ethoxy}methyl) morpholin-4-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 103F (50 mg) in dichloromethane (220 μL) was added trifluoroacetic acid (220 μL), and the reaction was allowed to stir for 4 hours. The reaction was concentrated under a stream of nitrogen and was taken up in water and acetonitrile. The mixture was purified by RP-HPLC on a Gilson PLC 2020 using a Luna™ column (250×50 mm, 10 mm) (5-85% over 30 minutes with acetonitrile in water containing 10 mM ammonium acetate) to give the title compound. ¹H NMR (400 MHz, dimethylsulfoxide-d₆) δ ppm 8.72 (s, 1H), 8.35 (d, 1H), 7.24-7.07 (m, 5H), 6.85-6.68 (m, 3H), 6.25-6.15 (m, 1H), 5.86-5.77 (m, 1H), 5.05-4.80 (m, 3H), 4.58-4.34 (m, 3H), 3.96-3.86 (m, 1H), 3.74-3.21 (m, 16H), 3.01-2.87 (m, 3H), 2.78-2.59 (m, 4H), 2.43 (br s, 4H), 2.21 (s, 3H), 2.01-1.92 (m, 6H). MS (ESI) m/z 1101.9 (M−H)⁻.

### Example 104

### (7R,16R)-19,23-dichloro-1-(4-fluorophenyl)-10-{[2-(3-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 104A

### 1-bromo-3-(2-(2-(2-methoxyethoxy)ethoxy)ethoxy)benzene

To a solution of 3-bromophenol (9.29 g) in acetonitrile (200 mL) was added 2-(2-(2-methoxyethoxy)ethoxy)ethyl 4-methylbenzenesulfonate (15 g) and potassium carbonate (18.56 g). The reaction mixture was stirred at 80° C. for 12 hours. The solution was filtered, and the filtrate was diluted with ethyl acetate (500 mL). The solution was washed with 15% aqueous sodium hydroxide (200 mL) three times, washed with brine (200 mL) twice and dried over anhydrous magnesium sulfate. The solution was filtered, concentrated under reduced pressure, and the material was purified by flash column chromatography on silica gel using a gradient of 10-100% ethyl acetate in petroleum ether. The solvent was removed under vacuum to yield the title compound. ¹H NMR (400 MHz, chloroform-d) δ ppm 7.16-7.10 (m, 1H), 7.10-7.05 (m, 2H), 6.85 (ddd, 1H), 4.13-4.09 (m, 2H), 3.87-3.83 (m, 2H), 3.76-3.72 (m, 2H), 3.70-3.65 (m, 4H), 3.57-3.54 (m, 2H), 3.38 (s, 3H). MS (ESI) m/z 319.0 (M+H)⁺.

### Example 104B

### 2-(3-(2-(2-(2-methoxyethoxy)ethoxy)ethoxy)phenyl)-4,4,5,5-tetramethyl-1,3,2-dioxaborolane

A mixture of 1-bromo-3-(2-(2-(2-methoxyethoxy)ethoxy)ethoxy)benzene(10 g), 4,4,4′,4′,5,5,5′,5′-octamethyl-2,2′-bi(1,3,2-dioxaborolane) (18.70 g), potassium acetate (7.23 g), 1,1′-bis(diphenylphosphino)ferrocenedichloro palladium (II) dichloromethane complex (1.077 g) in 1,4-dioxane (300 mL) was stirred at 80° C. for 12 hours. The solution was filtered, and the filtrate concentrated in vacuo and the residue was dissolved in ethyl acetate (400 mL). The solution was washed with saturated aqueous ammonium chloride (100 mL) three times. The solution was washed with brine (3×100 mL), dried over anhydrous magnesium sulfate, and filtered. The solution was concentrated under vacuum and purified by flash column chromatography on silica gel using a gradient of 2-100% ethyl acetate in petroleum ether. The solvent was removed under vacuum to yield the title compound. ¹H NMR (400 MHz, chloroform-d) δ ppm 7.51-7.16 (m, 3H), 7.03 (dd, 1H), 4.17 (t, 2H), 3.86 (t, 2H), 3.78-3.73 (m, 2H), 3.72-3.64 (m, 4H), 3.59-3.54 (m, 2H), 3.39 (s, 3H), 1.35 (s, 12H). MS (ESI) m/z 384.2 (M+NH₄)⁺.

### Example 104C

### (2-(3-(2-(2-(2-methoxyethoxy)ethoxy)ethoxy)phenyl)pyrimidin-4-yl)methanol

The title compound was prepared by substituting Example 104B for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.88 (d, 1H), 7.98 (d, 1H), 7.93 (s, 1H), 7.50 (d, 1H), 7.43 (t, 1H), 7.11 (dd, 1H), 5.67 (t, 1H), 4.64 (d, 2H), 4.17 (t, 2H), 3.78 (t, 2H), 3.62-3.60 (m, 2H), 3.56-3.51 (m, 4H), 3.44-3.42 (m, 2H), 3.23 (s, 3H). MS (ESI) m/z 349.3 (M+H)⁺.

### Example 104D

### (7R,16R)-19,23-dichloro-1-(4-fluorophenyl)-10-{[2-(3-{2-[2-(2-methoxyethoxy)ethoxy]ethoxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 104C for Example 38D in Example 38E. ¹H NMR (500 MHz, dimethylsulfoxide-d₆) δ ppm 8.89 (d, 1H), 8.74 (s, 1H), 8.00 (d, 1H), 7.95 (s, 1H), 7.54 (d, 1H), 7.43 (t, 1H), 7.22-7.11 (m, 5H), 6.89 (d, 1H), 6.75 (dd, 1H), 6.24 (m, 1H), 5.82 (s, 1H), 5.23 (q, 2H), 4.86 (m, 1H), 4.44 (m, 2H), 4.17 (t, 2H), 3.78 (t, 2H), 3.67 (dd, 1H), 3.63-3.60 (m, 2H), 3.55-3.50 (m, 6H), 3.22 (s, 3H), 2.90 (d, 2H), 2.67 (m, 3H), 2.48-2.34 (m, 6H), 2.18 (s, 3H), 1.99 (s, 3H), 1.96 (s, 3H). MS (ESI) m/z 1085.4 (M+H)⁺.

### Example 105

### (7R,16R)-19,23-dichloro-1-(4-fluorophenyl)-10-({2-[(1r,4r)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 105A

### (2-((1s,4s)-4-(2,5,8,11-tetraoxadodecyl)-4-fluorocyclohexyl)pyrimidin-4-yl)methanol

A mixture of Example 101I (300 mg), triethylamine (237 mg) and 10% Pd/C (33.2 mg) in dry tetrahydrofuran (30 mL) was stirred for 16 hours under H₂ (15 psi) at 25° C. and filtered. The filtrate was concentrated and the residue was purified by HPLC on a Gilson 281 semi-preparative HPLC system (Mobile phase: A: trifluoroacetic acid/water=0.075% v/v; B: acetonitrile; Column: Nano-micro Kromasil C18 100*30 mm 5 μm; Flow rate: 25 mL/minute; Monitor wavelength: 220 and 254 nm) to provide the title compound. ¹H NMR (400 MHz, CDCl₃) δ ppm 8.62 (d, 1H), 7.10 (d, 1H), 4.73 (s, 2H), 3.74-3.62 (m, 11H), 3.58-3.54 (m, 3H), 3.52 (s, 1H), 3.38 (s, 3H), 2.96-2.85 (m, 1H), 2.16-1.93 (m, 6H), 1.69-1.47 (m, 2H).

### Example 105B

### (2-((1r,4r)-4-(2,5,8,11-tetraoxadodecyl)-4-fluorocyclohexyl)pyrimidin-4-yl)methanol

The title compound was obtained during the HPLC purification in Example 105A. ¹H NMR (400 MHz, CDCl₃) δ ppm 8.62 (d, 1H), 7.12 (d, 1H), 4.73 (s, 2H), 3.57-3.49 (m, 15H), 3.37 (s, 3H), 3.12-3.01 (m, 1H), 2.17-2.01 (m, 4H), 1.95-1.83 (m, 2H), 1.83-1.71 (m, 2H).

### Example 105C

### tert-butyl (7R,16R)-19,23-dichloro-1-(4-fluorophenyl)-10-({2-[(1r,4r)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 105B. MS (ESI) m/z 1177.7 (M+H)⁺.

### Example 105D

### (7R,16R)-19,23-dichloro-1-(4-fluorophenyl)-10-({2-[(1r,4r)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 105C. ¹H NMR (500 MHz, dimethylsulfoxide-d₆) δ ppm 8.77-8.72 (m, 2H), 7.43 (d, 1H), 7.20 (t, 2H), 7.18-7.11 (m, 2H), 6.87 (d, 1H), 6.77 (dd, 1H), 6.24 (dd, 1H), 5.77 (d, 1H), 5.25-4.99 (m, 2H), 4.87 (t, 1H), 4.45 (d, 2H), 3.80-3.46 (m, 77H), 3.21 (s, 3H), 3.01-2.93 (m, 2H), 2.70 (dd, 2H), 2.30 (d, 4H), 2.01-1.89 (m, 10H), 1.82 (q, 2H), 1.72-1.61 (m, 2H). MS (ESI) m/z 1123.7 (M+H)⁺.

### Example 106

### (7R,16R)-19,23-dichloro-10-({2-[(4R*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 106A

### 8-fluoro-8-(2,5,8,11,14-pentaoxapentadecyl)-1,4-dioxaspiro[4.5]decane

The title compound was prepared as described in Example 101E by replacing 2-(2-(2-methoxyethoxy)ethoxy)ethyl 4-methylbenzenesulfonate with 2,5,8,11-tetraoxatridecan-13-yl 4-methylbenzenesulfonate.

### Example 106B

### 4-fluoro-4-(2,5,8,11,14-pentaoxapentadecyl)cyclohexanone

The title compound was prepared as described in Example 101F by replacing Example 101E with Example 106A.

### Example 106C

### 4-fluoro-4-(2,5,8,11,14-pentaoxapentadecyl)cyclohex-1-en-1-yl trifluoromethanesulfonate

The title compound was prepared as described in Example 101G by replacing Example 101F with Example 106B.

### Example 106D

### 2-(4-fluoro-4-(2,5,8,11,14-pentaoxapentadecyl)cyclohex-1-en-1-yl)-4,4,5,5-tetramethyl-1,3,2-dioxaborolane

The title compound was prepared as described in Example 101H by replacing Example 101G with Example 106C.

### Example 106E

### (2-(4-fluoro-4-(2,5,8,11,14-pentaoxapentadecyl)cyclohex-1-en-1-yl)pyrimidin-4-yl)methanol

The title compound was prepared as described in Example 101I by replacing Example 101H with Example 106D.

### Example 106F

### (S)-(2-(4-fluoro-4-(2,5,8,11,14-pentaoxapentadecyl)cyclohex-1-en-1-yl)pyrimidin-4-yl)methanol

The title compound was prepared as described in Example 101J by replacing Example 101I with Example 106E. ¹H NMR (400 MHz, CDCl₃) δ ppm 8.63 (d, 1H), 7.23 (br d, 1H), 7.07 (d, 1H), 4.74 (d, 2H), 3.76-3.61 (m, 16H), 3.57-3.53 (m, 2H), 3.38 (s, 3H), 2.79 (br s, 2H), 2.65-2.47 (m, 2H), 2.20-2.08 (m, 1H), 2.01-1.83 (m, 1H).

### Example 106G

### (R)-(2-(4-fluoro-4-(2,5,8,11,14-pentaoxapentadecyl)cyclohex-1-en-1-yl)pyrimidin-4-yl)methanol

The title compound was prepared as described in Example 101J by replacing Example 101I with Example 106E. ¹H NMR (400 MHz, CDCl₃) δ ppm 8.64 (d, 1H), 7.23 (br s, 1H), 7.07 (d, 1H), 4.74 (s, 2H), 3.76-3.61 (m, 16H), 3.58-3.53 (m, 2H), 3.38 (s, 3H), 2.79 (br s, 2H), 2.66-2.53 (m, 2H), 2.20-2.10 (m, 1H), 2.00-1.83 (m, 1H), 1.64 (br s, 1H).

### Example 106H

### tert-butyl (7R,16R)-19,23-dichloro-10-({2-[(4R*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 106G.

### Example 106I

### (7R,16R)-19,23-dichloro-10-({2-[(4R*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 106H. ¹H NMR (500 MHz, dimethylsulfoxide-d₆) δ ppm 8.76-8.70 (m, 2H), 7.42 (d, 1H), 7.19 (dd, 2H), 7.13 (td, 3H), 6.82 (d, 1H), 6.72 (dd, 1H), 6.20 (dd, 1H), 5.85 (d, 1H), 5.16 (d, 1H), 5.08 (d, 1H), 4.88 (d, 1H), 4.44 (d, 2H), 3.63-3.58 (m, 4H), 3.56 (t, 3H), 3.54-3.48 (m, 11H), 3.41 (dd, 2H), 3.22 (s, 3H), 2.94 (d, 1H), 2.76-2.60 (m, 4H), 2.44 (s, 3H), 2.22 (s, 3H), 2.07-2.00 (m, 1H), 1.97 (d, 6H), 1.78 (dq, 1H). MS (ESI) m/z 1163.5 (M+H)⁺.

### Example 107

### (7R,16R)-19,23-dichloro-1-(4-fluorophenyl)-20,22-dimethyl-16-[(4-methylpiperazin-1-yl)methyl]-10-[(2-{4-[(1,4,7,10,13-pentaoxacyclopentadecan-2-yl)methoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 107A

### 2-(4-((1,4,7,10,13-pentaoxacyclopentadecan-2-yl)methoxy)phenyl)-4,4,5,5-tetramethyl-1,3,2-dioxaborolane

4-(4,4,5,5-Tetramethyl-1,3,2-dioxaborolan-2-yl)phenol (700 mg), (1,4,7,10,13-pentaoxacyclopentadecan-2-yl)methanol (955 mg), and triphenylphosphine (1251 mg) were dissolved in tetrahydrofuran (14 mL). (E)-Diisopropyl diazene-1,2-dicarboxylate (965 mg) was added, and the solution was stirred at room temperature overnight. The solution was concentrated under vacuum and purified by flash column chromatography using a gradient of 30-100% ethyl acetate in heptanes. The solvent was removed under vacuum to yield the title compound. ¹H NMR (500 MHz, dimethylsulfoxide-d₆) δ ppm 7.60 (d, 2H), 6.94 (d, 2H), 4.06 (dd, 1H), 3.97 (dd, 1H), 3.85 (m, 1H), 3.75-3.66 (m, 3H), 3.62-3.52 (m, 15H), 1.27 (s, 12H). MS (ESI) m/z 470.3 (M+NH₄)⁺.

### Example 107B

### (2-(4-((1,4,7,10,13-pentaoxacyclopentadecan-2-yl)methoxy)phenyl)pyrimidin-4-yl)methanol

The title compound was prepared by substituting Example 107A for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.81 (d, 1H), 8.33 (d, 2H), 7.41 (d, 1H), 7.07 (d, 2H), 5.66 (t, 1H), 4.61 (d, 2H), 4.11 (dd, 1H), 4.05 (dd, 1H), 3.89 (m, 1H), 3.78-3.69 (m, 3H), 3.61 (dd, 2H), 3.56 (s, 12H), 3.17 (d, 1H). MS (ESI) m/z 435.1 (M+H)⁺.

### Example 107C

### (7R,16R)-19,23-dichloro-1-(4-fluorophenyl)-20,22-dimethyl-16-[(4-methylpiperazin-1-yl)methyl]-10-[(2-{4-[(1,4,7,10,13-pentaoxacyclopentadecan-2-yl)methoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 107B for Example 38D in Example 38E. ¹H NMR (500 MHz, dimethylsulfoxide-d₆) δ ppm 8.82 (d, 1H), 8.73 (s, 1H), 8.34 (d, 2H), 7.44 (d, 1H), 7.20 (t, 2H), 7.14 (dd, 2H), 7.07 (d, 2H), 6.88 (d, 1H), 6.75 (dd, 1H), 6.22 (m, 1H), 5.81 (s, 1H), 5.21 (q, 2H), 4.86 (m, 1H), 4.45 (m, 2H), 4.12 (dd, 1H), 4.04 (dd, 1H), 3.89 (m, 1H), 3.76-3.69 (m, 4H), 3.65-3.53 (m, 16H), 2.98 (dd, 2H), 2.69-2.62 (m, 2H), 2.48-2.38 (m, 2H), 2.37-2.28 (m, 4H), 2.15 (s, 3H), 1.98 (s, 3H), 1.96 (s, 3H). MS (ESI) m/z 1169.3 (M+H)⁺.

### Example 108

### (7R,16R)-10-[(2-{bis[2-(2-methoxyethoxy)ethyl]amino}pyrimidin-4-yl)methoxy]-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 108A

### (2-(bis(2-(2-methoxyethoxy)ethyl)amino)pyrimidin-4-yl)methanol

To a solution of (2-chloropyrimidin-4-yl)methanol (100 mg) in acetonitrile (5 mL) was added bis(2-(2-methoxyethoxy)ethyl)amine (200 mg) and triethylamine (0.5 mL). The reaction mixture was stirred overnight at 110° C. in a Biotage® Initiator microwave unit. The reaction mixture was concentrated in vacuo. To the residue was added ethyl acetate and the organic phase was washed with saturated aqueous ammonium chloride solution and water. The organic phase was then dried with sodium sulfate, filtered, and concentrated in vacuo. Purification by chromatography on silica gel using an ISCO CombiFlash® Companion MPLC (eluting with 0-10% methanol in dichloromethane) provided title compound. MS (APCI) m/z 330.2 (M+H)⁺.

### Example 108B

### tert butyl (7R,16R)-10-[(2-{bis[2-(2-methoxyethoxy)ethyl]amino}pyrimidin-4-yl)methoxy]-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (30 mg), Example 108A (15 mg), triphenylphosphine (33 mg) and (E)-N¹,N¹,N²,N²-tetramethyldiazene-1,2-dicarboxamide (22 mg) was purged for 30 minutes with argon. A mixture of toluene (0.5 mL) and tetrahydrofuran (0.5 mL) was added and the reaction mixture was stirred for 48 hours at ambient temperature. The reaction mixture was concentrated in vacuo. To the residue was added dichloromethane and the mixture was washed once with water. The organic phase was filtered through a Chromabond® PTS cartridge and the organic phase was concentrated in vacuo. The residue was purified by normal phase MPLC on a Teledyne-Isco-CombiFlash® system (eluting with 0-20% methanol in dichloromethane) to afford the title compound. MS (APCI) m/z 1121.4 (M+H)⁺.

### Example 108C

### (7R,16R)-10-[(2-{bis[2-(2-methoxyethoxy)ethyl]amino}pyrimidin-4-yl)methoxy]-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 108B (39 mg) in dichloromethane (0.5 mL) was added trifluoroacetic acid (200 μL). The reaction mixture was stirred overnight at ambient temperature. The reaction mixture was then concentrated in vacuo. To the residue was added acetone and the mixture was concentrated in vacuo. The process was repeated overall three times. The residue was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.73 (d, 1H), 8.30 (d, 1H), 7.20 (m, 2H), 7.14 (m, 2H), 6.80 (d, 1H), 6.72 (m, 2H), 6.18 (m, 1H), 5.80 (s, 1H), 4.92 (m, 1H), 4.88 (m, 2H), 4.44 (m, 2H), 3.73 (m, 4H), 3.56 (m, 5H), 3.51 (m, 4H), 3.42 (m, 4H), 3.22 (s, 6H), 2.93 (m, 1H), 2.68 (m, 2H), 2.55-2.25 (m, 8H), 2.18 (s, 3H), 1.97 (s, 6H). MS (ESI) m/z 1064.2 (M+H)⁺.

### Example 109

### (7R,16R)-10-({2-[bis(2,5,8,11-tetraoxatridecan-13-yl)amino]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 109A

### (2-(di(2,5,8,11-tetraoxatridecan-13-yl)amino)pyrimidin-4-yl)methanol

To a solution of (2-chloropyrimidin-4-yl)methanol (100 mg) in acetonitrile (5 mL) was added di(2,5,8,11-tetraoxatridecan-13-yl)amine (200 mg) and N,N-diisopropylethylamine (0.4 mL). The reaction mixture was stirred overnight at 110° C. in a Biotage® Initiator microwave unit. The reaction mixture was concentrated in vacuo. To the residue was added ethyl acetate and the organic phase was washed with saturated aqueous ammonium chloride solution and water. The organic phase was dried with sodium sulfate, filtered, and concentrated in vacuo. Purification by chromatography on silica gel using an ISCO CombiFlash® Companion MPLC (eluting with 0-10% methanol in dichloromethane) provided title compound. MS (APCI) m/z 506.30 (M+H)⁺.

### Example 109B

### tert butyl (7R,16R)-10-({2-[bis(2,5,8,11-tetraoxatridecan-13-yl)amino]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, charged with Example 16N (30 mg), Example 109A (20 mg) triphenylphosphine (33 mg) and (E)-N¹,N¹,N²,N²-tetramethyldiazene-1,2-dicarboxamide (22 mg) was purged for 30 minutes with argon. A mixture of toluene (0.5 mL) and tetrahydrofuran (0.5 mL) was added and the reaction mixture was stirred for 48 hours at ambient temperature. The reaction mixture was concentrated in vacuo. To the residue was added dichloromethane and the mixture was washed once with water. The organic phase was filtered through a Chromabond® PTS cartridge and the organic phase was concentrated in vacuo. The residue was first purified by normal phase MPLC on a Teledyne-Isco-CombiFlash® system (eluting with 0-20% methanol in dichloromethane) and purified again by normal phase MPLC on a Teledyne-Isco-CombiFlash® system (eluting with 0-15% methanol in dichloromethane) to afford the title compound. MS (APCI) m/z 1296.5 (M+H)⁺.

### Example 109C

### (7R,16R)-10-({2-[bis(2,5,8,11-tetraoxatridecan-13-yl)amino]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 109B (39 mg) in dichloromethane (0.5 mL) was added trifluoroacetic acid (150 μL). The reaction mixture was stirred overnight at ambient temperature. The reaction mixture was then concentrated in vacuo. To the residue was added acetone and the mixture was concentrated in vacuo. The residue was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.73 (s, 1H), 8.31 (d, 1H), 7.20 (m, 2H), 7.14 (m, 2H), 6.81 (d, 1H), 6.71 (m, 2H), 6.19 (m, 1H), 5.80 (s, 1H), 4.92 (m, 1H), 4.88 (m, 2H), 4.44 (m, 2H), 3.74 (m, 4H), 3.57 (m, 5H), 3.50 (m, 20H), 3.41 (m, 4H), 3.22 (s, 6H), 2.93 (m, 1H), 2.68 (m, 2H), 2.55-2.25 (m, 8H), 2.17 (s, 3H), 1.97 (s, 6H). MS (ESI) m/z 1240.3 (M+H)⁺.

### Example 110

### (7R,16R)-19,23-dichloro-10-[(2-{4-[(1,3-dimethoxypropan-2-yl)oxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 110A

### 2-(4-((1,3-dimethoxypropan-2-yl)oxy)phenyl)-4,4,5,5-tetramethyl-1,3,2-dioxaborolane

1,3-Dimethoxypropan-2-ol (328 mg), 4-hydroxyphenylboronic acid pinacol ester (200 mg). N,N,N′,N′-tetramethylazodicarboxamide (626 mg) and triphenylphosphine (953 mg) were combined and flushed with argon for 15 minutes. Tetrahydrofuran (1.0 mL) and toluene (1.0 mL) were also flushed with argon for 15 minutes and then combined with the reactants. The mixture was stirred overnight at room temperature. The reaction mixture was partitioned between dichloromethane and water. The aqueous layer was extracted with dichloromethane (twice). The combined organic layers were washed with brine and dried over sodium sulfate, filtered, and concentrated. Purification was performed on a silica gel column (12 g, 0-30% methanol in dichloromethane). The desired fractions were combined and the solvents were removed under reduced pressure to provide the title compound. MS (ESI) m/z 323.2 (M+H)⁺.

### Example 110B

### (2-(4-((1,3-dimethoxypropan-2-yl)oxy)phenyl)pyrimidin-4-yl)methanol

Example 110A (293 mg), (2-chloropyrimidin-4-yl)methanol (131 mg), and tetrakis(triphenylphosphine)palladium (53 mg) were dissolved in tetrahydrofuran (6.0 mL). Aqueous sodium bicarbonate solution (6 mL, 9%) was added under argon atmosphere. The reaction was heated for 4 hours at 120° C. in a Biotage® Initiator microwave reactor. The reaction mixture was diluted with ethyl acetate and water. The aqueous layer was washed with ethyl acetate (three times). The combined organic layers were dried over magnesium sulfate, filtered, and concentrated. Purification was performed on a silica gel column (12 g, 0-40% ethyl acetate in n-heptane). The desired fractions were combined and the solvents were removed under reduced pressure to provide the title compound. MS (ESI) m/z 305.2 (M+H)⁺.

### Example 110C

### tert-butyl (7R,16R)-19,23-dichloro-10-[(2-{4-[(1,3-dimethoxypropan-2-yl)oxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 110B (21 mg), Example 16N (25 mg), triphenylphosphine (32 mg), and N,N,N′,N′-tetramethylazodicarboxamide (21 mg) were combined and flushed with argon for 15 minutes. Tetrahydrofuran (1.0 mL) and toluene (1.0 mL) were mixed, flushed with argon for 15 minutes, and added to the reactants. The reaction mixture was stirred at room temperature over the weekend. The reaction mixture was concentrated. Purification was performed on a silica gel column (4 g, 0-70% ethyl acetate in n-heptane, then 100% methanol). The desired fractions were combined and the solvents were removed under reduced pressure to provide the title compound. MS (APCI) m/z 1195.4 (M+H)⁺.

### Example 110D

### (7R,16R)-19,23-dichloro-10-[(2-{4-[(1,3-dimethoxypropan-2-yl)oxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 110C (70 mg) was dissolved in dichloromethane (1.0 mL) and trifluoroacetic acid (245 μL) was added. The reaction mixture was stirred at room temperature overnight. The reaction mixture was concentrated at 25° C. The residue was dissolved in methanol, diluted with water, and freeze-dried. The crude material was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.82 (d, 1H), 8.73 (s, 1H), 8.34-8.32 (m, 2H), 7.45 (d, 1H), 7.22-7.19 (m, 2H), 7.15-7.10 (m, 4H), 6.87 (d, 1H), 6.75-6.73 (m, 1H), 6.20 (m, 1H), 5.82 (m, 1H), 5.24 (d, 1H), 5.17 (d, 1H), 4.87 (m, 1H), 4.75-4.71 (m, 1H), 4.46-4.41 (m, 2H), 3.65-3.62 (m, 1H), 3.57-3.55 (m, 4H), 3.28 (s, 6H), 2.99-2.96 (m, 1H), 2.70-2.63 (m, 2H), 2.56-2.25 (m, 8H), 2.14 (s, 3H), 1.98 (s, 3H), 1.96 (s, 3H). MS (APCI) m/z 1039.1 (M+H)⁺.

### Example 111

### (7R,16R)-19,23-dichloro-1-(4-fluorophenyl)-10-({2-[(4R*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 111A

### tert-butyl (7R,16R)-19,23-dichloro-1-(4-fluorophenyl)-10-({2-[(4R*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 101K. MS (ESI) m/z 1177.6 (M+H)⁺.

### Example 111B

### (7R,16R)-19,23-dichloro-1-(4-fluorophenyl)-10-({2-[(4R*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 111A. ¹H NMR (400 MHz, dimethylsulfoxide-d₆) δ ppm 8.67 (d, 2H), 7.33 (d, 1H), 7.18-7.03 (m, 5H), 6.78 (d, 1H), 6.69 (dd, 1H), 6.18 (dd, 1H), 5.71 (d, 1H), 5.10 (d, 1H), 5.02 (d, 1H), 4.82-4.75 (m, 1H), 4.38 (d, 2H), 3.59 (d, 1H), 3.57-3.52 (m, 4H), 3.47 (dtd, 11H), 3.35 (dd, 2H), 3.16 (s, 3H), 2.90 (d, 1H), 2.63 (d, 2H), 2.48 (s, 2H), 2.38 (s, 3H), 2.17 (s, 3H), 1.97 (d, 1H), 1.93 (s, 3H), 1.88 (s, 3H), 1.82-1.60 (m, 1H). MS (ESI) m/z 1121.6 (M+H)⁺.

### Example 112

### (7R,16R)-19,23-dichloro-1-(4-fluorophenyl)-20,22-dimethyl-16-[(4-methylpiperazin-1-yl)methyl]-10-({2-[(4R*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 112A

### (R)-2-(4-(2,5,8,11-tetraoxadodecyl)-4-methylcyclohex-1-en-1-yl)-4-(((tert-butyldimethylsilyl)oxy)methyl)pyrimidine

The title compound was prepared as described in Example 84G by replacing 2,5,8,11,14-pentaoxahexadecan-16-yl 4-methylbenzenesulfonate with 2-(2-(2-methoxyethoxy)ethoxy)ethyl 4-methylbenzenesulfonate.

### Example 112B

### (R)-(2-(4-(2,5,8,11-tetraoxadodecyl)-4-methylcyclohex-1-en-1-yl)pyrimidin-4-yl)methanol

The title compound was prepared as described in Example 84H by replacing Example 84G with Example 112A. ¹H NMR (400 MHz, CDCl₃) δ ppm 8.62 (d, 1H), 7.30 (br s, 1H), 7.03 (d, 1H), 4.73 (s, 2H), 3.72-3.64 (m, 8H), 3.63-3.58 (m, 2H), 3.58-3.54 (m, 2H), 3.39 (s, 3H), 3.31-3.22 (m, 2H), 2.73-2.52 (m, 2H), 2.31 (br dd, 1H), 2.05 (br d, 1H), 1.78-1.67 (m, 1H), 1.61-1.51 (m, 1H), 1.00 (s, 3H).

### Example 112C

### tert-butyl (7R,16R)-19,23-dichloro-1-(4-fluorophenyl)-20,22-dimethyl-16-[(4-methylpiperazin-1-yl)methyl]-10-({2-[(4R*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 112B. MS (ESI) m/z 587.3 (M+H)²⁺.

### Example 112D

### (7R,16R)-19,23-dichloro-1-(4-fluorophenyl)-20,22-dimethyl-16-[(4-methylpiperazin-1-yl)methyl]-10-({2-[(4R*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 112C. ¹H NMR (500 MHz, dimethylsulfoxide-d₆) δ ppm 8.66-8.61 (m, 2H), 7.32 (d, 1H), 7.17-7.12 (m, 2H), 7.12-7.03 (m, 3H), 6.75 (d, 1H), 6.65 (dd, 1H), 6.11 (t, 1H), 5.77 (d, 1H), 5.12-4.94 (m, 2H), 4.81 (d, 1H), 4.37 (d, 2H), 3.53 (dd, 2H), 3.48-3.42 (m, 10H), 3.34 (dd, 2H), 3.15 (s, 4H), 3.11 (d, 1H), 2.87 (d, 1H), 2.66-2.54 (m, 2H), 2.48 (s, 1H), 2.40 (s, 1H), 2.38 (s, 2H), 2.31 (s, 2H), 2.12 (s, 3H), 1.90 (d, 7H), 1.53 (dt, 1H), 1.39 (dt, 1H), 0.84 (s, 3H). MS (ESI) m/z 1115.5 (M+H)⁺.

### Example 113

### (7R,16R)-19,23-dichloro-10-({2-[(1s,4s)-4-fluoro-4-(2,5,8,11,14,17-hexaoxaoctadecan-1-yl)cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 113A

### (2-((1s,4s)-4-fluoro-4-(2,5,8,11,14,17-hexaoxaoctadecyl)cyclohexyl)pyrimidin-4-yl)methanol

The title compound was prepared as described in Example 105A by replacing Example 101I with Example 97E.

### Example 113B

### tert-butyl (7R,16R)-19,23-dichloro-10-({2-[(1s,4s)-4-fluoro-4-(2,5,8,11,14,17-hexaoxaoctadecan-1-yl)cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 113A. MS (ESI) m/z 633.5 (M+H)²⁺.

### Example 113C

### (7R,16R)-19,23-dichloro-10-({2-[(1s,4s)-4-fluoro-4-(2,5,8,11,14,17-hexaoxaoctadecan-1-yl)cyclohexyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 113B. ¹H NMR (500 MHz, dimethylsulfoxide-d₆) δ ppm 8.67-8.61 (m, 2H), 7.40 (d, 1H), 7.16-7.09 (m, 2H), 7.09-7.02 (m, 2H), 6.75 (d, 1H), 6.63 (dd, 1H), 6.08 (dd, 1H), 5.81 (d, 1H), 5.06 (d, 1H), 4.99 (d, 1H), 4.84 (d, 1H), 4.36 (d, 2H), 3.53-3.42 (m, 19H), 3.39 (s, 1H), 3.36-3.33 (m, 2H), 3.16 (s, 3H), 2.90-2.83 (m, 1H), 2.78 (p, 1H), 2.66-2.54 (m, 2H), 2.39 (s, 1H), 2.32 (s, 2H), 2.12 (s, 3H), 1.93 (s, 3H), 1.87 (d, 6H), 1.77 (td, 4H), 1.50 (ddt, 2H). MS (ESI) m/z 1209.6 (M+H)⁺.

### Example 114

### (7R,16R)-19,23-dichloro-10-({2-[(4S*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 114A

### tert-butyl (7R,16R)-19,23-dichloro-10-({2-[(4S*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 106F. MS (ESI) m/z 610.4 (M+H)²⁺.

### Example 114B (7R,16R)-19,23-dichloro-10-({2-[(4S*)-4-fluoro-4-(2,5,8,11,14-pentaoxapentadecan-1-yl)cyclohex-1-en-1-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 114A. ¹H NMR (500 MHz, dimethylsulfoxide-d₆) δ ppm 8.74 (d, 2H), 7.41 (d, 1H), 7.23-7.18 (m, 2H), 7.18-7.10 (m, 4H), 6.83 (d, 1H), 6.73 (dd, 1H), 6.21 (dd, 1H), 5.81 (d, 1H), 5.16 (d, 1H), 5.08 (d, 1H), 4.86 (p, 1H), 4.44 (d, 2H), 3.65-3.58 (m, 4H), 3.58-3.48 (m, 14H), 3.41 (dd, 2H), 3.22 (s, 3H), 2.99-2.91 (m, 1H), 2.81-2.61 (m, 3H), 2.39 (dd, 8H), 2.20 (s, 3H), 2.07-2.00 (m, 1H), 1.98 (s, 3H), 1.96 (s, 3H), 1.86-1.70 (m, 1H). MS (ESI) m/z 1163.6 (M+H)⁺.

### Example 115

### (7R,16R)-19,23-dichloro-1-(4-fluorophenyl)-20,22-dimethyl-16-[(4-methylpiperazin-1-yl)methyl]-10-{[2-(1,4,7,10-tetraoxa-13-azacyclopentadecan-13-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 115A

### (2-(1,4,7,10-tetraoxa-13-azacyclopentadecan-13-yl)pyrimidin-4-yl)methanol

1,4,7,10-Tetraoxa-13-azacyclopentadecane (250 mg), (2-chloropyrimidin-4-yl)methanol (150 mg) and triethylamine (315 mg) were dissolved in acetonitrile (4 mL). The solution was heated to 80° C. overnight. The solution was then cooled and concentrated under vacuum. The material was purified by flash column chromatography using a gradient of 0-5% methanol in dichloromethane. The solvent was removed under vacuum to yield the title compound. ¹H NMR (500 MHz, dimethylsulfoxide-d₆) δ ppm 8.31 (d, 1H), 6.70 (d, 1H), 5.36 (t, 1H), 4.34 (d, 2H), 3.67 (m, 8H), 3.55-3.51 (m, 12H). MS (ESI) m/z 328.3 (M+H)⁺, 326.0 (M−H)⁻.

### Example 115B

### (7R,16R)-19,23-dichloro-1-(4-fluorophenyl)-20,22-dimethyl-16-[(4-methylpiperazin-1-yl)methyl]-10-{[2-(1,4,7,10-tetraoxa-13-azacyclopentadecan-13-yl)pyrimidin-4-yl]methoxy}-7,8,15,16-tetrahydro-18,21-etheno-13,9-(metheno)-6,14,17-trioxa-2-thia-3,5-diazacyclononadeca

The title compound was prepared by substituting Example 115A for Example 38D in Example 38E. ¹H NMR (500 MHz, dimethylsulfoxide-d₆) δ ppm 8.73 (s, 1H), 8.31 (d, 1H), 7.22-7.12 (m, 4H), 6.83 (d, 1H), 6.74-6.68 (m, 2H), 6.20 (m, 1H), 5.80 (s, 1H), 4.93 (q, 2H), 4.89 (m, 1H), 4.44 (m, 2H), 3.83 (m, 1H), 3.68 (m, 8H), 3.54-3.50 (m, 12H), 2.94 (d, 2H), 2.68 (m, 3H), 2.46 (m, 2H), 2.38 (m, 4H), 2.19 (s, 3H), 1.97 (s, 3H), 1.96 (s, 3H). MS (ESI) m/z 1062.3 (M+H)⁺.

### Example 116

### (7R,16R)-19,23-dichloro-1-(4-fluorophenyl)-20,22-dimethyl-16-[(4-methylpiperazin-1-yl)methyl]-10-{[2-(1,4,7,10,13-pentaoxa-16-azacyclooctadecan-16-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 116A

### (2-(1,4,7,10,13-pentaoxa-16-azacyclooctadecan-16-yl)pyrimidin-4-yl)methanol

A 10 mL microwave tube was charged with (2-chloropyrimidin-4-yl)methanol (140 mg), 1,4,7,10,13-pentaoxa-16-azacyclooctadecane (265 mg) and acetonitrile (5 mL). N,N-Diisopropylethylamine (0.25 mL) was added, the vessel was capped and heated in a Biotage® microwave for 2 hours to 90° C. and for 4 hours to 105° C. Water (5 mL) and dichloromethane (50 mL) were added, the mixture stirred for 10 minutes, the layers separated via Chromabond® PTS cartridge and the organic layer concentrated in vacuo. Purification by chromatography using an ISCO CombiFlash® Companion MPLC (24 g RediSep® Gold column, eluting with 0-50% dichloromethane/methanol; in a second step 15 g Chromabond® RP-C18 column, eluting with 0-100% water/acetonitrile) gave the title compound. MS (APCI) m/z 372.2 (M+H)⁺.

### Example 116B

### tert-butyl (7R,16R)-19,23-dichloro-1-(4-fluorophenyl)-20,22-dimethyl-16-[(4-methylpiperazin-1-yl)methyl]-10-{[2-(1,4,7,10,13-pentaoxa-16-azacyclooctadecan-16-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 89C by replacing Example 89B with Example 116A. Purification by chromatography using an ISCO CombiFlash® Companion MPLC (12 g RediSep® Gold column, eluting with 0-50% dichloromethane/methanol; in a second step 15 g Chromabond® RP-C18 column, eluting with 0-100% water/acetonitrile+0.1% ammonium hydroxide) provided the title compound. MS (APCI) m/z 1162.4 (M+H)⁺.

### Example 116C

### (7R,16R)-19,23-dichloro-1-(4-fluorophenyl)-20,22-dimethyl-16-[(4-methylpiperazin-1-yl)methyl]-10-{[2-(1,4,7,10,13-pentaoxa-16-azacyclooctadecan-16-yl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 89D by replacing Example 89C with Example 116B. Purification by HPLC (XBridge C8 19×150 mm 5 μm column, gradient 5-100% acetonitrile+0.2% ammonium hydroxide in water+0.1% ammonium hydroxide) provided the title compound. ¹H NMR (600 MHz, dimethylsulfoxide-d₆) δ ppm 12.93 (s, 1H), 8.72 (s, 1H), 8.30 (d, 1H), 7.20 (dd, 2H), 7.13 (m, 2H), 6.80 (m, 1H), 6.70 (m, 2H), 6.16 (m, 1H), 5.82 (m, 1H), 4.96 (d, 1H), 4.89 (m, 2H), 4.44 (m, 2H), 3.76 (m, 4H), 3.58 (m, 4H), 3.53 (m, 16H), 2.92 (d, 1H), 2.73-2.63 (m, 2H), 2.55-2.45 (m, 9H), 2.34 (s, 3H), 2.17 (s, 3H), 1.97 (s, 6H). MS (APCI) m/z 1106.6 (M+H)⁺.

### Example 117

### (7R,16R)-19,23-dichloro-10-[(2-{3-[(1,1-dioxo-1λ⁶-thiomorpholin-4-yl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 117A

### 4-(3-(4-(hydroxymethyl)pyrimidin-2-yl)benzyl)thiomorpholine 1,1-dioxide

The title compound was prepared by substituting 4-(3-(4,4,5,5-tetramethyl-1,3,2-dioxaborolan-2-yl)benzyl)thiomorpholine 1,1-dioxide for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.88 (d, 1H), 8.34 (m, 1H), 8.30 (m, 1H), 7.50 (m, 3H), 5.70 (t, 1H), 4.65 (d, 2H), 3.77 (s, 2H), 3.12 (m, 4H), 2.92 (m, 4H). MS (ESI)m/z 334.3 (M+H)⁺.

### Example 117B

### (7R,16R)-19,23-dichloro-10-[(2-{3-[(1,1-dioxo-1λ⁶-thiomorpholin-4-yl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 117A for Example 38D in Example 38E. ¹H NMR (500 MHz, dimethylsulfoxide-d₆) δ ppm 8.89 (d, 1H), 8.75 (s, 1H), 8.36 (s, 1H), 8.32 (dd, 1H), 7.54-7.48 (m, 3H), 7.22-7.12 (m, 4H), 6.89 (d, 1H), 6.75 (dd, 1H), 6.24 (m, 1H), 5.82 (s, 1H), 5.24 (q, 2H), 4.86 (m, 1H), 4.44 (m, 2H), 3.77 (s, 2H), 3.66 (dd, 1H), 3.12 (m, 4H), 3.09 (d, 2H), 2.92 (m, 4H), 2.68 (m, 3H), 2.46-2.30 (m, 6H), 2.17 (s, 3H), 1.99 (s, 3H), 1.96 (s, 3H). MS (ESI) m/z 1068.3 (M+H)⁺.

### Example 118

### (7R,16R)-19,23-dichloro-1-(4-fluorophenyl)-10-({2-[(1s,4s)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 118A

### tert-butyl (7R,16R)-19,23-dichloro-1-(4-fluorophenyl)-10-({2-[(1s,4s)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 105A. MS (ESI) m/z 1177.6 (M+H)⁺.

### Example 118B

### (7R,16R)-19,23-dichloro-1-(4-fluorophenyl)-10-({2-[(1s,4s)-4-fluoro-4-(2,5,8,11-tetraoxadodecan-1-yl)cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 118A. ¹H NMR (500 MHz, dimethylsulfoxide-d₆) δ ppm 8.75-8.70 (m, 2H), 7.44 (d, 1H), 7.24-7.16 (m, 2H), 7.16-7.10 (m, 2H), 6.85 (d, 1H), 6.74 (dd, 1H), 6.21 (dd, 1H), 5.82 (d, 1H), 5.14 (d, 1H), 5.07 (d, 1H), 4.87 (p, 1H), 4.44 (d, 2H), 3.64-3.56 (m, 2H), 3.53 (tdd, 11H), 3.46 (s, 1H), 3.43 (dd, 2H), 3.24 (s, 3H), 2.95 (d, 2H), 2.85 (p, 1H), 2.68 (qd, 2H), 2.48-2.39 (m, 8H), 2.23 (s, 3H), 2.02-1.91 (m, 9H), 1.84 (td, 4H), 1.57 (ddt, 2H). MS (ESI) m/z 1121.2 (M+H)⁺.

### Example 119

### (7R,16R)-19,23-dichloro-1-(4-fluorophenyl)-20,22-dimethyl-16-[(4-methylpiperazin-1-yl)methyl]-10-({2-[(4S*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 119A

### (S)-2-(4-(2,5,8,11-tetraoxadodecyl)-4-methylcyclohex-1-en-1-yl)-4-(((tert-butyldimethylsilyl)oxy)methyl)pyrimidine

The title compound was prepared as described in Example 84G by replacing 2,5,8,11,14-pentaoxahexadecan-16-yl 4-methylbenzenesulfonate and Example 84E with 2-(2-(2-methoxyethoxy)ethoxy)ethyl 4-methylbenzenesulfonate and Example 84F, respectively.

### Example 119B

### (S)(2-(4-(2,5,8,11-tetraoxadodecyl)-4-methylcyclohex-1-en-1-yl)pyrimidin-4-yl)methanol

The title compound was prepared as described in Example 84H by replacing Example 84G with Example 119A. ¹H NMR (400 MHz, CDCl₃) δ ppm 8.62 (d, 1H), 7.29 (br t, 1H), 7.04 (d, 1H), 4.73 (s, 2H), 3.71-3.64 (m, 8H), 3.71-3.64 (m, 1H), 3.71-3.64 (m, 1H), 3.62-3.58 (m, 2H), 3.57-3.53 (m, 2H), 3.38 (s, 3H), 3.31-3.20 (m, 2H), 2.73-2.52 (m, 2H), 2.30 (br dd, 1H), 2.13-1.99 (m, 1H), 1.77-1.67 (m, 1H), 1.59-1.51 (m, 1H), 0.99 (s, 3H).

### Example 119C

### tert-butyl (7R,16R)-19,23-dichloro-1-(4-fluorophenyl)-20,22-dimethyl-16-[(4-methylpiperazin-1-yl)methyl]-10-({2-[(4S*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J with Example 119B. MS (ESI) m/z 1171.6 (M+H)⁺.

### Example 119D

### (7R,16R)-19,23-dichloro-1-(4-fluorophenyl)-20,22-dimethyl-16-[(4-methylpiperazin-1-yl)methyl]-10-({2-[(4S*)-4-methyl-4-(2,5,8,11-tetraoxadodecan-1-yl)cyclohex-1-en-1-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 119C. ¹H NMR (500 MHz, dimethylsulfoxide-d₆) δ ppm 8.72-8.68 (m, 2H), 7.40 (d, 1H), 7.23-7.18 (m, 3H), 7.18-7.09 (m, 3H), 6.80 (d, 1H), 6.69 (dd, 1H), 6.14 (s, 1H), 5.87 (d, 1H), 5.14 (d, 1H), 5.06 (d, 1H), 4.90 (d, 1H), 4.43 (d, 2H), 3.61-3.48 (m, 11H), 3.43-3.39 (m, 2H), 3.22 (s, 4H), 3.17 (d, 1H), 2.93 (d, 1H), 2.68 (td, 2H), 2.56-2.52 (m, 1H), 2.48-2.31 (m, 7H), 2.18 (s, 4H), 1.99 (s, 3H), 1.94 (s, 3H), 1.90 (s, 1H), 1.60 (dt, 1H), 1.46 (dt, 1H), 0.91 (s, 3H). MS (ESI) m/z 1113.0 (M−H)⁻.

### Example 120

### (7R,16R)-19,23-dichloro-1-(4-fluorophenyl)-20,22-dimethyl-16-[(4-methylpiperazin-1-yl)methyl]-10-({2-[(1R*,2R*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 120A

### methyl 2-(4,4,5,5-tetramethyl-1,3,2-dioxaborolan-2-yl)cyclohex-1-enecarboxylate

A 1000 mL 3-neck oven dried flask was charged with bis(pinacolato)diboron (20.83 g), triphenylarsine (1.748 g), and (1,5-cyclooctadiene)(methoxy) iridium (I) dimer (0.946 g). The vessel was flow purged with argon for 25 minutes. Octane (446 mL) was added, and the stirring reaction was heated to 85° C. and stirred overnight. The mixture was allowed to cool to ambient temperature and was filtered through diatomaceous earth. The mixture was concentrated by rotary evaporation and the crude residue was purified by flash column chromatography using a Teledyne Isco CombiFlash® instrument using a RediSep® Gold 120 g column eluting with 0 to 30% ethyl acetate/heptanes to yield the title compound. ¹H NMR (501 MHz, chloroform-d) δ ppm 3.74 (s, 3H), 2.23 (tq, 4H), 1.67-1.61 (m, 2H), 1.58 (dtt, 2H), 1.33 (s, 12H). MS (ESI) m/z 167.2 [M-pinacol+OH]⁺.

### Example 120B

### 2-chloro-4-(((triisopropylsilyl)oxy)methyl)pyrimidine

A 250 mL oven dried flask was charged with (2-chloropyrimidin-4-yl)methanol (8.0 g), and imidazole (7.91 g) in acetonitrile (70 mL) and N,N-dimethylformamide (10 mL). The reaction was stirred and cooled in an ice/water bath. Chlorotriisopropylsilane (12.19 mL) was added and the water bath was removed. The mixture was concentrated by rotary evaporation and treated with tert-butyl methyl ether (250 mL). Water (250 mL) was added, the layers were separated, and the aqueous layer was extracted with tert-butyl methyl ether (100 mL). The combined organic layers were washed with water and saturated aqueous sodium chloride solution, dried over anhydrous magnesium sulfate, filtered and concentrated to give the title compound. ¹H NMR (501 MHz, chloroform-d) δ ppm 8.64 (d, 1H), 7.58 (dt, 1H), 4.87 (s, 2H), 1.26-1.14 (m, 3H), 1.10 (d, 18H). MS (DCI) m/z 301.1 [M+H]⁺.

### Example 120C

### methyl 2-(4-(((triisopropylsilyl)oxy)methyl)pyrimidin-2-yl)cyclohex-1-ene-1-carboxylate

To a 250 mL three neck flask was added tris(dibenzylideneacetone) dipalladium (0) (0.242 g), (1S,3R,5R,7S)-1,3,5,7-tetramethyl-8-phenyl-2,4,6-trioxa-8-phosphaadamantane (0.170 g) and potassium phosphate (7.85 g). The flask was flushed with nitrogen for 30 minutes. A 250 mL flask was charged with Example 120B (5.3 g) and Example 120A (5.63 g). Tetrahydrofuran (70 mL) and water (18 mL) were added, and the stirring mixture was sparged with nitrogen for 30 minutes, and transferred to the reaction flask via cannula. The reaction was warmed to 65° C. and stirred overnight. The reaction mixture was allowed to cool to room temperature and treated with ammonium pyrrolidine-1-carbodithioate (2.89 g). Water (5 mL) and ethyl acetate (10 mL) were added and the mixture was stirred for 30 minutes before filtering through a pad of diatomaceous earth. The layers were separated and the organic layer was washed sequentially with saturated aqueous sodium bicarbonate and saturated aqueous sodium chloride, dried over anhydrous magnesium sulfate and concentrated under reduced pressure. The residue was purified by flash column chromatography using an Analogix 280 SF with a 330 g column eluting with a 0-45% tert-butyl methyl ether/heptanes gradient to give the title compound. ¹H NMR (501 MHz, chloroform-d) δ ppm 8.70 (d, 1H), 7.45 (dd, 1H), 4.85 (s, 2H), 3.57 (s, 3H), 2.60 (dq, 2H), 2.45 (tt, 2H), 1.24-1.14 (m, 3H), 1.10 (d, 18H). MS (ESI) m/z 405.2 [M+H]⁺.

### Example 120D

### (1S,2R)-methyl 2-(4-(((triisopropylsilyl)oxy)methyl)pyrimidin-2-yl)cyclohexanecarboxylate

A stainless steel reactor was charged with Example 120C (10.85 g), methanol (90 mL) and 5% Pd/C (wet JM #9, 918 mg). The reactor was purged with nitrogen. The mixture was stirred under 50 psi of hydrogen at 25° C. for 68 hours. The reactor was vented and the mixture filtered through a pad of diatomaceous earth. The material was concentrated then purified by flash column chromatography using an Analogix280 SF with a 330 g column eluting with a 0-45% tert-butyl methyl ether/heptanes gradient. The enantiomers were separated on a Thar SFC80 preparative SFC system using a Chiralpak IC 250×30 mm i.d. 5 μM column with a flow rate of 64 g/minute, a system back pressure of 100 bar, a column temperature of 33° C., and a mobile phase of 20% methanol in CO₂ to provide the title compound as the second eluting peak. ¹H NMR (400 MHz, chloroform-d) δ ppm 8.65 (d, 1H), 7.37 (dt, 1H), 4.80 (s, 2H), 3.53 (d, 3H), 3.41 (dt, 1H), 3.08 (dt, 1H), 2.34 (dtd, 1H), 2.10 (dtd, 1H), 2.00 (ddt, 1H), 1.86 (ddt, 1H), 1.68 (dtd, 1H), 1.56-1.36 (m, 3H), 1.30-1.13 (m, 3H), 1.10 (d, 18H). MS (ESI) m/z 407.3 [M+H]⁺.

### Example 120E

### (1R,2S)-methyl 2-(4-(((triisopropylsilyl)oxy)methyl)pyrimidin-2-yl)cyclohexanecarboxylate

The title compound was also obtained from the preparation of 120D, isolated as the first eluting peak. ¹H NMR (400 MHz, chloroform-d) δ ppm 8.65 (d, 1H), 7.37 (dt, 1H), 4.80 (s, 2H), 3.53 (d, 3H), 3.41 (dt, 1H), 3.08 (dt, 1H), 2.34 (dtd, 1H), 2.10 (dtd, 1H), 2.00 (ddt, 1H), 1.86 (ddt, 1H), 1.68 (dtd, 1H), 1.56-1.36 (m, 3H), 1.30-1.13 (m, 3H), 1.10 (d, 18H). MS (ESI) m/z 407.3 [M+H]⁺.

### Example 120F

### (1R,2R)-methyl 2-(4-(((triisopropylsilyl)oxy)methyl)pyrimidin-2-yl)cyclohexanecarboxylate

To Example 120D (500 mg) was slowly added sodium methoxide (0.5 M in methanol, 12.3 mL). The mixture was stirred at 70° C. for 1 day. The mixture was subsequently diluted with water, acidified with acetic acid and extracted with dichloromethane. The combined organic phases were dried over sodium sulfate, filtered and concentrated. The crude product was purified by silica gel flash chromatography on an AnaLogix IntelliFlash²⁸⁰ system (solvent A=3:1 ethyl acetate:ethanol; solvent B=heptane, eluting with 0-40% A to B) to afford the title compound. ¹H NMR (400 MHz, dimethylsulfoxide-d₆) δ ppm 8.71 (d, 1H), 7.34 (d, 1H), 4.84-4.65 (m, 2H), 3.39 (s, 3H), 3.06-2.77 (m, 2H), 1.99 (dt, 2H), 1.76 (dd, 2H), 1.50-1.08 (m, 7H), 1.04 (d, 18H). MS (ESI) m/z 407.4 (M+H)⁺.

### Example 120G

### ((1R,2R)-2-(4-(((triisopropylsilyl)oxy)methyl)pyrimidin-2-yl)cyclohexyl)methanol

To a solution of Example 120F (100 mg) in tetrahydrofuran (5 mL) at 0° C. was added (slowly over 8 minutes) lithium aluminum hydride (1.0M in tetrahydrofuran, 0.246 mL). The mixture was stirred at 0° C. for 30 minutes. The reaction mixture was then quenched by slow addition of ethyl acetate and methanol, then diluted with saturated aqueous Rochelle's salt solution and stirred for 30 minutes. The organic layer was separated and concentrated. The residue was purified by silica gel flash chromatography on AnaLogix IntelliFlash²⁸⁰ system eluting with 0-5% methanol in dichloromethane to give the title compound. MS (ESI) m/z 379.3 (M+H)⁺.

### Example 120H

### 2-((1R,2R)-2-(2,5,8,11,14-pentaoxapentadecyl)cyclohexyl)-4-(((triisopropylsilyl)oxy)methyl)pyrimidine

To a stirring solution of Example 120G (80 mg) in tetrahydrofuran (3.0 mL) was slowly added sodium hydride (16.90 mg) and the mixture was stirred for 25 minutes. 2,5,8,11-Tetraoxatridecan-13-yl 4-methylbenzenesulfonate (153 mg) was added and the reaction was stirred at 50° C. for 7 hours. One drop of saturated aqueous ammonium chloride solution was added and the reaction was filtered to remove the material. The solids were washed with dichloromethane. The organics were concentrated and purified by silica gel flash chromatography (solvent A=3:1 ethyl acetate:ethanol; solvent B=Heptane, eluting with 5-50% A to B) to give the title compound. MS (ESI) m/z 569.4 (M+H)⁺.

### Example 1201

### (2-((1R,2R)-2-(2,5,8,11,14-pentaoxapentadecyl)cyclohexyl)pyrimidin-4-yl)methanol

To a solution of Example 120H (102 mg) in tetrahydrofuran (2 mL) was added tetrabutylammonium fluoride (1.0M in tetrahydrofuran, 0.215 mL). The mixture was stirred for 30 minutes. The mixture was concentrated and purified by silica gel flash chromatography on AnaLogix IntelliFlash²⁸⁰ system (solvent A=3:1 ethyl acetate:ethanol; solvent B=heptanes, eluting with 15-80% A to B) to give the title compound. LC/MS (ESI) m/z 413.5 (M+H)⁺.

### Example 120J

### tert-butyl (7R,16R)-19,23-dichloro-1-(4-fluorophenyl)-20,22-dimethyl-16-[(4-methylpiperazin-1-yl)methyl]-10-({2-[(1R*,2R*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 1021 for Example 88F in Example 88G. MS (ESI) m/z 1203.5 (M+H)⁺.

### Example 120K

### (7R,16R)-19,23-dichloro-1-(4-fluorophenyl)-20,22-dimethyl-16-[(4-methylpiperazin-1-yl)methyl]-10-({2-[(1R*,2R*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 120J for Example 51E in Example 51F. ¹H NMR (400 MHz, dimethylsulfoxide-d₆) δ ppm 8.72 (s, 1H), 8.70 (d, 1H), 7.42 (d, 1H), 7.24-7.09 (m, 4H), 6.85 (d, 1H), 6.73 (dd, 1H), 6.21 (dd, 1H), 5.82 (d, 1H), 5.08 (q, 2H), 4.90 (q, 1H), 4.44 (d, 2H), 3.74-2.25 (m, 34H), 2.20 (s, 3H), 2.09 (dq, 1H), 1.98 (s, 3H), 1.96 (s, 3H), 1.94-1.84 (m, 1H), 1.75 (d, 3H), 1.55 (d, 1H), 1.38-1.08 (m, 3H). MS (ESI) m/z 1147.3 (M+H)⁺.

### Example 121

### (7R,16R)-19,23-dichloro-10-[(2-{(1r,4r)-4-[(1,4-dioxan-2-yl)methoxy]-1-[2-(2-methoxyethoxy)ethoxy]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 121A

### 4-(2-(2-methoxyethoxy)ethoxy)cyclohexanone

Sodium hydride (2.53 g, 60% in mineral oil) was added to 1,4-dioxaspiro[4.5]decan-8-ol (8.8 g) in 80 mL tetrahydrofuran and the reaction was stirred for 45 minutes. 3-Bromoprop-1-ene (7 mL) was then added and the reaction was stirred for 24 hours. The reaction mixture was cooled and poured into ethyl acetate, washed with water, dried over sodium sulfate, filtered, and concentrated. The crude material was chromatographed on silica gel using 1-50% ethyl acetate in heptanes as eluent to give the title compound. ¹H NMR (400 MHz, dimethylsulfoxide-d₆) δ ppm 5.88 (ddt, 1H), 5.23 (dd, 1H), 5.10 (dd, 1H), 3.93 (m, 2H), 3.84 (m, 4H), 3.40 (m, 1H), 1.73 (m, 2H), 1.65 (m, 2H), 1.56 (m, 2H), 1.45 (m, 2H).

### Example 121B

### 3-(1,4-dioxaspiro[4.5]decan-8-yloxy)propane-1,2-diol

AD-Mix-b (67.1 g, 1.4 g/mmol) was taken up in 200 mL tert-butanol and 200 ml water, cooled to 0° C. and Example 121A (9.5 g) was added. The mixture was allowed to warm to room temperature overnight. Sodium sulfite (70 g) was added and the mixture was stirred for 1 hour. The reaction was poured into ethyl acetate, washed with 1M aqueous sodium hydroxide solution, water and brine, dried over sodium sulfate, filtered and concentrated. The crude material was chromatographed on silica gel using 10-100% ethyl acetate in heptanes as eluent to give the title compound. ¹H NMR (400 MHz, dimethylsulfoxide-d₆) δ ppm 4.55 (d, 1H), 4.43 (t, 1H), 3.84 (s, 4H), 3.52 (m, 1H), 3.36 (m, 2H), 3.28 (m, 3H), 1.75-1.60 (m, 4H), 1.56 (m, 2H), 1.44 (m, 2H). MS (ESI) m/z 233.2 (M+H)⁺.

### Example 121C

### 8-((1,4-dioxan-2-yl)methoxy)-1,4-dioxaspiro[4.5]decane

NaH (2.79 g) was added to a solution of Example 121B (13.5 g) in 200 mL tetrahydrofuran and the reaction was stirred for 10 minutes. Example 91E (12.50 g) was added and the reaction was stirred overnight. The mixture was quenched with ammonium chloride solution and extracted twice with ethyl acetate, washed with brine, dried over sodium sulfate, filtered and concentrated. The crude material was chromatographed on silica gel using 2-30% ethyl acetate in heptanes as eluent to give the title compound. ¹H NMR (400 MHz, dimethylsulfoxide-d₆) δ ppm 3.83 (s, 4H), 3.71 (dd, 1H), 3.68 (dd, 1H), 3.63-3.52 (m, 3H), 3.43 (dt, 1H), 3.35 (m, 2H), 3.31 (dd, 1H), 3.26 (dd, 1H), 1.70 (m, 2H), 1.63 (m, 2H), 1.53 (m, 2H), 1.44 (m, 2H). MS (ESI) m/z 259.1 (M+H)⁺.

### Example 121D

### 4-((1,4-dioxan-2-yl)methoxy)cyclohexanone

Example 121C (7.3 g) was taken up in 200 mL acetone, p-toluenesulfonic acid monohydrate (5.38 g), was added, and the solution was heated to reflux for 5 days. The solution was cooled and poured into ethyl acetate, and the solution was washed with saturated aqueous sodium carbonate solution and brine, dried over sodium sulfate, filtered, and concentrated. The crude material was chromatographed on silica gel using 2-50% ethyl acetate in heptanes as eluent to give the title compound. ¹H NMR (400 MHz, dimethylsulfoxide-d₆) δ ppm 3.73 (dd, 1H), 3.68 (dd, 1H), 3.63 (m, 2H), 3.56 (dt, 1H), 3.46 (m, 1H), 3.40 (dd, 2H), 3.31 (dd, 1H), 2.34 (m, 2H), 2.19 (m, 2H), 1.90 (m, 4H).

### Example 121E

### 4-((1,4-dioxan-2-yl)methoxy)-1-hydroxycyclohexanecarbonitrile

A solution of sodium bisulfite (1.544 g) in 20 mL water was added in portions to Example 121D (2.65 g) and potassium cyanide (1.208 g) in 30 mL water, and the reaction was stirred for 2 hours. Ethyl acetate was added, the layers were separated, and the organic layer was dried over sodium sulfate, filtered, and concentrated to give the title compound. ¹H NMR (400 MHz, dimethylsulfoxide-d₆) δ ppm 6.50 (s, 0.5H), 6.43 (s, 0.5H), 3.72 (m, 1H), 3.57 (m, 2H), 3.54 (m, 4H), 3.43 (m, 2H), 3.24 (s, 3H), 2.36 (m, 2H), 2.20 (m, 2H), 1.90 (m, 4H). MS (ESI) m/z 264.2 (M+Na)⁺.

### Example 121F

### (1r,4r)-4-((1,4-dioxan-2-yl)methoxy)-1-(2-(2-methoxyethoxy)ethoxy)cyclohexane-1-carbonitrile

Zinc chloride (2.02 g) was heated at 120° C. under vacuum overnight, and cooled. 2-(2-Methoxy)ethanol (2.29 g) was added, Example 121E (2.98 g) was added, and the reaction was heated to 70° C. for six days. The mixture was cooled and 2 mL methanol was added. The crude mixture was purified by reverse phase chromatography using a 10-60% gradient of acetonitrile in water (with 0.1% ammonium acetate) over 30 minutes on a Grace Reveleris equipped with a Luna™ column: C18(2), 100 Å, 250×50 mm to isolate the title compound. ¹H NMR (400 MHz, dimethylsulfoxide-d₆) δ ppm 3.70 (m, 2H), 3.64 (m, 1H), 3.60 (m, 1H), 3.56 (m, 1H), 3.52 (m, 1H), 3.43 (m, 1H), 3.38 (m, 1H), 3.33 (dd, 1H), 3.31 (sm, 8H), 3.25 (dd, 1H), 3.24 (s, 3H), 2.07 (m, 2H), 1.85 (m, 2H), 1.71 (m, 2H), 1.42 (m, 2H). MS (ESI) m/z 361.1 (M+NH₄)⁺.

### Example 121G

### (1S,4r)-4-((1,4-dioxan-2-yl)methoxy)-1-(2-(2-methoxyethoxy)ethoxy)cyclohexanecarboximidamide acetate

The title compound was prepared by substituting Example 121F for Example 74A in Example 74B. MS (ESI) m/z 361.0 (M+H)⁺.

### Example 121H

### (2-((1S,4r)-4-((1,4-dioxan-2-yl)methoxy)-1-(2-(2-methoxyethoxy)ethoxy)cyclohexyl)pyrimidin-4-yl)methanol

The title compound was prepared by substituting Example 121G for Example 74B in Example 74C. ¹H NMR (400 MHz, dimethylsulfoxide-d₆) δ ppm 8.80 (d, 1H), 7.45 (d, 1H), 5.63 (t, 1H), 4.56 (d, 2H), 3.68 (m, 2H), 3.56 (m, 4H), 3.45 (m, 4H), 3.37 (m, 4H), 3.24 (m, 2H), 3.21 (s, 3H), 3.16 (m, 2H), 2.24 (m, 2H), 1.79 (m, 4H), 1.43 (m, 2H). MS (ESI) m/z 427.2 (M+H)⁺.

### Example 121I

### (7R,16R)-19,23-dichloro-10-[(2-{(1r,4r)-4-[(1,4-dioxan-2-yl)methoxy]-1-[2-(2-methoxyethoxy)ethoxy]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was prepared by substituting Example 121H for Example 38D in Example 38E. ¹H NMR (400 MHz, dimethylsulfoxide-d₆) δ ppm 8.74 (d, 1H), 8.71 (s, 1H), 7.53 (dd, 1H), 7.19 (m, 2H), 7.13 (m, 2H), 6.84 (dd, 1H), 6.72 (dd, 1H), 6.20 (d, 1H), 5.85 (d, 1H), 5.12 (dd, 2H), 4.90 (m, 1H), 4.44 (d, 2H), 3.67 (m, 2H), 3.56 (m, 4H), 3.50 (m, 4H), 3.44 (m, 4H), 3.36 (m, 4H), 3.30 (m, 3H), 3.24 (m, 4H), 3.19 (s, 3H), 2.94 (m, 1H), 2.68 (m, 2H), 2.44 (m, 4H), 2.27 (m, 2H), 2.04 (s, 3H), 1.98 (s, 3H), 1.96 (s, 3H), 1.79 (m, 4H), 1.58 (m, 2H), 1.42 (m, 2H). MS (ESI) m/z 1161.5 (M+H)⁺.

### Example 122

### (7R,16R)-19,23-dichloro-10-[(2-{(1s,4s)-4-[(1,4-dioxan-2-yl)methoxy]-1-[2-(2-methoxyethoxy)ethoxy]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 122A

### (1 s,4s)-4-((1,4-dioxan-2-yl)methoxy)-1-(2-(2-methoxyethoxy)ethoxy)cyclohexane-1-carbonitrile

The title compound was isolated from Example 121F as the opposite diastereomer. ¹H NMR (400 MHz, dimethylsulfoxide-d₆) δ ppm 3.70 (m, 2H), 3.65 (m, 2H), 3.57 (m, 2H), 3.55 (m, 3H), 3.44 (m, 2H), 3.37 (m, 1H), 3.33 (m, 4H), 3.28 (m, 2H), 3.24 (s, 3H), 1.90 (m, 4H), 1.63 (m, 4H). MS (ESI) m/z 361.2 (M+NH₄)⁺.

### Example 122B

### (1S,4s)-4-((1,4-dioxan-2-yl)methoxy)-1-(2-(2-methoxyethoxy)ethoxy)cyclohexanecarboximidamide acetate

The title compound was prepared by substituting Example 122A for Example 74A in Example 74B. MS (ESI) m/z 361.0 (M+H)⁺.

### Example 122C

### (2-((1S,4S)-4-((1,4-dioxan-2-yl)methoxy)-1-(2-(2-methoxyethoxy)ethoxy)cyclohexyl)pyrimidin-4-yl)methanol

The title compound was prepared by substituting Example 122B for Example 74B in Example 74C. ¹H NMR (400 MHz, dimethylsulfoxide-d₆) δ ppm 8.78 (d, 1H), 7.44 (d, 1H), 5.63 (br s, 1H), 4.54 (s, 2H), 3.70 (m, 2H), 3.61 (m, 4H), 3.43 (m, 4H), 3.38 (m, 4H), 3.24 (m, 2H), 3.22 (s, 3H), 3.19 (m, 2H), 2.13 (m, 2H), 1.85 (m, 2H), 1.69 (m, 2H), 1.58 (m, 2H). MS (ESI) m/z 427.2 (M+H)⁺.

### Example 122D

### (7R,16R)-19,23-dichloro-10-[(2-{(1r,4r)-4-[(1,4-dioxan-2-yl)methoxy]-1-[2-(2-methoxyethoxy)ethoxy]cyclohexyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was prepared by substituting Example 122C for Example 38D in Example 38E. ¹H NMR (400 MHz, dimethylsulfoxide-d₆) δ ppm 8.81 (d, 1H), 8.75 (s, 1H), 7.52 (dd, 1H), 7.19 (m, 2H), 7.14 (m, 2H), 6.88 (dd, 1H), 6.79 (dd, 1H), 6.25 (d, 1H), 5.78 (d, 1H), 5.12 (dd, 2H), 4.92 (m, 1H), 4.45 (d, 2H), 3.71 (m, 2H), 3.60 (m, 4H), 3.50 (m, 4H), 3.42 (m, 4H), 3.36 (m, 4H), 3.30 (m, 3H), 3.24 (m, 4H), 3.20 (s, 3H), 2.98 (m, 1H), 2.77 (m, 2H), 2.58 (m, 4H), 2.50 (s, 3H), 2.14 (m, 2H), 1.98 (s, 3H), 1.96 (s, 3H), 1.88 (m, 4H), 1.69 (m, 2H), 1.59 (m, 2H). MS (ESI) m/z 1161.5 (M+H)⁺.

### Example 123

### (7R,16R)-19,23-dichloro-1-(4-fluorophenyl)-10-[(2-{4-[(1,4,7,10,13,16-hexaoxacyclooctadecan-2-yl)methoxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 123A

### 2-(4-((1,4,7,10,13,16-hexaoxacyclooctadecan-2-yl)methoxy)phenyl)-4,4,5,5-tetramethyl-1,3,2-dioxaborolane

The title compound was prepared by substituting (1,4,7,10,13,16-hexaoxacyclooctadecan-2-yl)methanol for (1,4,7,10,13-pentaoxacyclopentadecan-2-yl)methanol in Example 107A. ¹H NMR (500 MHz, dimethylsulfoxide-d₆) δ ppm 7.60 (d, 2H), 6.94 (d, 2H), 4.08 (dd, 1H), 4.02 (dd, 1H), 3.84 (m, 1H), 3.71 (m, 2H), 3.60 (d, 2H), 3.57-3.51 (m, 18H), 1.27 (s, 12H). MS (ESI) m/z 514.4 (M+NH₄)⁺.

### Example 123B

### (2-(4-((1,4,7,10,13,16-hexaoxacyclooctadecan-2-yl)methoxy)phenyl)pyrimidin-4-yl)methanol

The title compound was prepared by substituting Example 123A for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.81 (d, 1H), 8.33 (d, 2H), 7.41 (d, 1H), 7.07 (d, 2H), 5.64 (t, 1H), 4.61 (d, 2H), 4.17-4.07 (m, 3H), 3.88 (m, 1H), 3.74 (m, 2H), 3.64-3.62 (m, 2H), 3.58-3.52 (m, 15H), 3.17 (d, 2H). MS (ESI) m/z 479.4 (M+H)⁺.

### Example 123C

### (7R,16R)-19,23-dichloro-1-(4-fluorophenyl)-10-[(2-{4-[(1,4,7,10,13,16-hexaoxacyclooctadecan-2-yl)methoxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 123B for Example 38D in Example 38E. ¹H NMR (500 MHz, dimethylsulfoxide-d₆) δ ppm 8.82 (d, 1H), 8.70 (s, 1H), 8.34 (d, 2H), 7.47 (d, 1H), 7.19 (t, 2H), 7.13 (dd, 2H), 7.08 (d, 2H), 6.85 (d, 1H), 6.71 (m, 1H), 6.17 (m, 1H), 5.87 (s, 1H), 5.20 (q, 2H), 4.89 (m, 1H), 4.44 (m, 2H), 4.14 (dd, 1H), 4.09 (dd, 1H), 3.88 (m, 1H), 3.74 (m, 2H), 3.63 (d, 2H), 3.61-3.58 (m, 2H), 3.56-3.53 (m, 17H), 2.97 (d, 2H), 2.66 (m, 3H), 2.44 (m, 2H), 2.37-2.30 (m, 4H), 2.14 (s, 3H), 1.99 (s, 3H), 1.95 (s, 3H). MS (ESI) m/z 1215.4 (M+H)⁺.

### Example 124

### (7R,16R)-19,23-dichloro-1-(4-fluorophenyl)-20,22-dimethyl-16-[(4-methylpiperazin-1-yl)methyl]-10-({2-[(1S*,2S*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 124A

### (1S,2S)-methyl 2-(4-(((triisopropylsilyl)oxy)methyl)pyrimidin-2-yl)cyclohexanecarboxylate

The title compound was prepared by substituting Example 120E for Example 120D in Example 120F. MS (ESI) m/z 407.4 (M+H)⁺.

### Example 124B

### ((1S,2S)-2-(4-(((triisopropylsilyl)oxy)methyl)pyrimidin-2-yl)cyclohexyl)methanol

The title compound was prepared by substituting Example 124A for Example 120F in Example 120G. MS (ESI) m/z 379.4 (M+H)⁺.

### Example 124C

### 2-((1S,2S)-2-(2,5,8,11,14-pentaoxapentadecyl)cyclohexyl)-4-(((triisopropylsilyl)oxy)methyl)pyrimidine

The title compound was prepared by substituting Example 124B for Example 120G in Example 120H. MS (ESI) m/z 569.6 (M+H)⁺.

### Example 124D

### (2-((1S,2S)-2-(2,5,8,11,14-pentaoxapentadecyl)cyclohexyl)pyrimidin-4-yl)methanol

The title compound was prepared by substituting Example 124C for Example 120H in Example 120I. LC/MS (ESI) m/z 413.2 (M+H)⁺.

### Example 124E

### tert-butyl (7R,16R)-19,23-dichloro-1-(4-fluorophenyl)-20,22-dimethyl-16-[(4-methylpiperazin-1-yl)methyl]-10-({2-[(1S*,2S*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 124D for Example 1201 in Example 120J. MS (ESI) m/z 1203.3 (M+H)⁺.

### Example 124F

### (7R,16R)-19,23-dichloro-1-(4-fluorophenyl)-20,22-dimethyl-16-[(4-methylpiperazin-1-yl)methyl]-10-({2-[(1S*,2S*)-2-(2,5,8,11,14-pentaoxapentadecan-1-yl)cyclohexyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 124E for Example 120J in Example 120K. ¹H NMR (501 MHz, dimethylsulfoxide-d₆) δ ppm 8.73 (s, 1H), 8.70 (d, 1H), 7.42 (d, 1H), 7.23-7.10 (m, 4H), 6.85 (d, 1H), 6.73 (dd, 1H), 6.21 (dd, 1H), 5.82 (d, 1H), 5.17-5.00 (m, 2H), 4.89 (p, 1H), 4.53-4.33 (m, 2H), 3.68-3.28 (m, 34H), 2.20 (s, 3H), 2.09 (dq, 1H), 1.97 (s, 3H), 1.96 (s, 3H), 1.91 (d, 1H), 1.77 (dd, 3H), 1.55 (q, 1H), 1.30 (t, 2H), 1.12 (d, 1H). MS (ESI) m/z 1147.6 (M+H)⁺.

### Example 125

### (7R,16R)-19,23-dichloro-1-(cyclopent-1-en-1-yl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 125A

### (R)-tert-butyl 2-acetoxy-3-(5-((tert-butyldimethylsilyl)oxy)-2-hydroxyphenyl)propanoate

To a solution of Example 12C (12 g) in tetrahydrofuran (300 mL) was added Pd/C (0.210 g) under a nitrogen atmosphere. The suspension was degassed and purged with hydrogen three times. The reaction mixture was stirred under 50 psi of hydrogen at 50° C. for 10 hours. The mixture was cooled, filtered and concentrated to give a residue which was purified by column chromatography on silica gel (eluting with petroleum ether:ethyl acetate=100:1 to 100:5) to give the title compound. ¹H NMR (400 MHz, CDCl₃) δ ppm 6.71-6.69 (m, 1H), 6.64-6.61 (m, 2H), 5.55 (s, 1H), 5.19-5.15 (dd, 1H), 3.14-3.02 (m, 2H), 2.12 (s, 3H), 1.43 (s, 9H), 0.97 (s, 9H), 0.17 (s, 6H).

### Example 125B

### (R)-tert-butyl 2-acetoxy-3-(5-((tert-butyldimethylsilyl)oxy)-2-((2-(trimethylsilyl)ethoxy)methoxy)phenyl)propanoate

To a solution of Example 125A (8.8 g) in tetrahydrofuran (280 mL) was added sodium hydride (0.120 g, 60% dispersion) at 0° C. After 15 minutes. (2-(chloromethoxy)ethyl)-trimethylsilane (0.810 g) was added into the mixture dropwise. The reaction was stirred at 25° C. for 12 hours under a nitrogen atmosphere. One additional vial was set up as described above and both of the two mixtures were combined. The reaction was quenched with water and extracted with ethyl acetate three times. The combined organic layers were washed with brine twice, dried over anhydrous sodium sulfate, filtered and concentrated to give a residue which was purified by column chromatography on silica gel (petroleum ether:ethyl acetate=100:1 to 100:5) to give the title compound. ¹H NMR (400 MHz, CDCl₃) δ ppm 6.97-6.95 (m, 1H), 6.67-6.64 (m, 2H), 5.20-5.12 (m, 3H), 3.79-3.75 (m, 2H), 3.20-3.15 (dd, 1H), 2.97-2.91 (dd, 1H), 2.05 (s, 3H), 1.43 (s, 9H), 0.99-0.94 (m, 11H), 0.17-0.16 (m, 6H), 0.03-0.00 (m, 9H).

### Example 125C

### (R)-tert-butyl 3-(5-((tert-butyldimethylsilyl)oxy)-2-((2-(trimethylsilyl)ethoxy)methoxy)phenyl)-2-hydroxypropanoate

To a solution of Example 125B (9 g) in ethyl alcohol (280 mL) was added sodium ethanolate (6.3 mg) at 0° C. under nitrogen flow. After 15 minutes, the reaction mixture was stirred at 25° C. for 1 hour. The reaction was quenched with water and extracted with ethyl acetate three times. The combined organic layers were washed with brine twice, dried over anhydrous sodium sulfate, filtered and concentrated to give a residue which was purified by column chromatography on silica gel (petroleum ether:ethyl acetate=100:1 to 100:5) to give the title compound. ¹H NMR (400 MHz, CDCl₃) δ ppm 6.96 (d, 1H), 6.70-6.63 (m, 2H), 5.18 (s, 2H), 4.36-4.31 (m, 1H), 3.79-3.75 (m, 2H), 3.04-2.90 (m, 3H), 1.43 (s, 9H), 0.99-0.95 (m, 11H), 0.17 (s, 6H), 0.04-0.01 (m, 9H).

### Example 125D

### 4-chloro-5-(3,5-dichloro-4-methoxy-2,6-dimethylphenyl)thieno[2,3-d]pyrimidine

To a suspension of Example 15E (25 g) in acetonitrile (300 mL) was added N-chlorosuccinimide (24 g) and HBF₄·Et₂O (tetrafluoroboric acid diethyl ether complex, 29 g). The reaction mixture was stirred at 15° C. under nitrogen atmosphere for 16 hours. Another reaction was set up as above, and the two reaction mixtures were combined. The reaction mixture was diluted with water and extracted with ethyl acetate three times. The combined organic layers were dried over anhydrous sodium sulfate, filtered and concentrated. The residue was purified by column chromatography on silica gel (petroleum:ethyl acetate from 200:1 to 20:1) to give the title compound. ¹H NMR (400 MHz, dimethylsulfoxide-d₆) δ ppm 9.01 (s, 1H), 8.02 (s, 1H), 3.88 (s, 3H), 2.01 (s, 6H).

### Example 125E

### 4-chloro-5-(3,5-dichloro-4-methoxy-2,6-dimethylphenyl)-6-iodothieno[2,3-d]pyrimidine

To a suspension of Example 125D (20 g) in tetrahydrofuran (200 mL) was added lithium diisopropylamide (38.1 mL, 2M) at −78° C. under nitrogen, and the reaction was stirred for 0.5 hours. Iodine (19.4 g) in tetrahydrofuran (100 mL) was added, and the reaction mixture was stirred at the same temperature for 0.5 hours. The reaction mixture was warmed to 15° C. under nitrogen atmosphere for 1 hour. Two other vials were set up as described above. The three reactions were combined, and the resulting mixture was treated with saturated aqueous sodium thiosulfate and extracted with ethyl acetate three times. The combined organic layers were dried over anhydrous sodium sulfate, filtered and concentrated. The crude material was purified by column chromatography on silica gel (petroleum ether:ethyl acetate from 100:1 to 40:1) to give the title compound. ¹H NMR (400 MHz, dimethylsulfoxide-d₆) δ ppm 8.96 (s, 1H), 3.90 (s, 3H), 1.95 (s, 6H).

### Example 125F

### 2,6-dichloro-4-(4-chloro-6-iodothieno[2,3-d]pyrimidin-5-yl)-3,5-dimethylphenol

To a solution of Example 125E (7.5 g) in dichloroethane (100 mL) was added aluminum chloride (6.0 g) at 0° C. and the reaction was heated at 68° C. for 6 hours. Two additional vials were set up as described above. The three reactions were combined, and the resulting mixture was quenched with saturated aqueous sodium bicarbonate and saturated aqueous ammonium chloride at 0° C. The mixture was extracted with ethyl acetate/tetrahydrofuran=1:1 three times, and the combined organic phases were washed with brine, dried over anhydrous magnesium sulfate filtered and concentrated under vacuum. The residue was purified by column chromatography on silica gel (n-hexane/ethyl acetate/tetrahydrofuran=20:1:1 to 10:1:1) to give the title compound. ¹H NMR (400 MHz, CDCl₃) δ ppm 8.85 (s, 1H), 6.23 (s, 1H), 2.00 (s, 6H).

### Example 125G

### 2,6-dichloro-4-(4-chloro-6-(cyclopent-1-en-1-yl)thieno[2,3-d]pyrimidin-5-yl)-3,5-dimethylphenol

To a suspension of Example 125F (2.3 g) and 2-(cyclopent-1-en-1-yl)-4,4,5,5-tetramethyl-1,3,2-dioxaborolane (1.3 g) in water (5 mL) and dioxane (50 mL) was added cesium carbonate (3 g) and tetrakis(triphenylphosphine)palladium (0) (0.535 g). The reaction mixture was heated to 80° C. under nitrogen atmosphere for 2 hours. The resulting mixture was diluted with water and extracted with ethyl acetate three times. The combined organic layers were washed with brine, dried over anhydrous sodium sulfate, filtered and concentrated under vacuum. The residue was purified by column chromatography on silica gel (n-hexane/ethyl acetate=100:1 to 15:1) to give the title compound. ¹H NMR (400 MHz, dimethylsulfoxide-d₆) δ ppm 10.13 (br s, 1H), 8.71-9.01 (m, 1H), 6.10 (d, 1H), 2.39 (td, 2H), 2.08-2.17 (m, 2H), 1.94 (s, 6H), 1.80 (quin, 2H).

### Example 125H

### (R)-5-(4-((1-(allyloxy)-3-(bis(4-methoxyphenyl)(phenyl)methoxy) propan-2-yl)oxy)-3,5-dichloro-2,6-dimethylphenyl)-4-chloro-6-(cyclopent-1-en-1-yl)thieno[2,3-d]pyrimidine

To a suspension of Example 125G (6.6 g) and Example 15J (9.4 g) in tetrahydrofuran (80 mL) was added triphenylphosphine (8.1 g) and (E)-di-tert-butyl diazene-1,2-dicarboxylate (7.1 g) at 0° C. The reaction mixture was warmed to 25° C. and stirred for 12 hours. The reaction was concentrated to give a residue which was purified by column chromatography on silica gel (eluting with petroleum ether:ethyl acetate=94:6) to give the title compound. ¹H NMR (400 MHz, CDCl₃) δ ppm 8.79 (s, 1H), 7.47 (d, 2H), 7.35 (d, 4H), 7.31-7.25 (m, 3H), 7.19 (dd, 2H), 6.87-6.77 (m, 5H), 5.95 (br s, 1H), 5.88-5.74 (m, 1H), 5.26-5.07 (m, 2H), 4.81-4.70 (m, 1H), 3.96 (d, 2H), 3.90-3.83 (m, 2H), 3.81-3.77 (m, 7H), 3.53 (d, 2H), 2.42-2.32 (m, 2H), 2.19 (br t, 2H), 2.01 (d, 6H), 1.89-1.77 (m, 3H).

### Example 125I

### (R)-tert-butyl 2-((5-(4-(((R)-1-(allyloxy)-3-(bis(4-methoxyphenyl) (phenyl)methoxy) propan-2-yl)oxy)-3,5-dichloro-2,6-dimethylphenyl)-6-(cyclopent-1-en-1-yl)thieno[2,3-d]pyrimidin-4-yl)oxy)-3-(5-((tert-butyldimethylsilyl)oxy)-2-((2-(trimethylsilyl)ethoxy)methoxy)phenyl)propanoate

To a suspension of Example 125H (4.8 g) and Example 125C (3.3 g) in tert-butanol (60 mL) was added cesium carbonate (6.6 g) at 25° C. under nitrogen flow. The reaction mixture was stirred at 65° C. for 16 hours. The reaction was quenched with water and extracted with ethyl acetate three times. The combined organic layers were washed with brine twice, dried over anhydrous sodium sulfate, filtered and concentrated to give a residue which was purified by column chromatography on silica gel (eluting with petroleum ether:ethyl acetate=95:5) to give the title compound which was used to the next step without further purification.

### Example 125J

### (R)-tert-butyl 2-((5-(4-(((S)-1-(allyloxy)-3-hydroxypropan-2-yl)oxy)-3,5-dichloro-2,6-dimethylphenyl)-6-(cyclopent-1-en-1-yl)thieno[2,3-d]pyrimidin-4-yl)oxy)-3-(5-((tert-butyldimethylsilyl)oxy)-2-((2-(trimethylsilyl)ethoxy)methoxy)phenyl)propanoate

To a solution of Example 125I (3.5 g) in methanol (25 mL) and dichloromethane (25 mL) was added formic acid (4.1 mL) at 0° C. The reaction was stirred at 25° C. for 16 hours. Three additional vials were set up as described above, and all the four reaction mixtures were combined. The combined mixture was poured into saturated aqueous sodium bicarbonate solution at 0° C. and extracted with ethyl acetate three times. The combined organic phases were washed with brine twice, dried over anhydrous sodium sulfate, filtered and concentrated to get the crude product. The crude product was purified by column chromatography on silica gel (eluting with petroleum ether:ethyl acetate=97:3 to 90:10) to give the title compound. ¹H NMR (400 MHz, CDCl₃) δ ppm 8.48 (s, 1H), 7.35-7.24 (m, 2H), 7.21-7.14 (m, 1H), 6.91 (d, 1H), 6.87-6.79 (m, 1H), 6.61 (dd, 1H), 6.38 (d, 1H), 5.94-5.77 (m, 2H), 5.34 (t, 1H), 5.23 (dd, 1H), 5.19-5.09 (m, 3H), 4.59-4.50 (m, 1H), 4.04-3.93 (m, 3H), 3.92-3.79 (m, 5H), 3.78-3.70 (m, 5H), 2.58 (d, 2H), 2.51 (dd, 1H), 2.45-2.36 (m, 2H), 2.27-2.15 (m, 5H), 2.00 (s, 3H), 1.92-1.80 (m, 5H), 1.27 (s, 11H), 1.02-0.82 (m, 14H), 0.10 (d, 6H), 0.01 (s, 9H).

### Example 125K

### (R)-tert-butyl 2-((5-(4-(((R)-1-(allyloxy)-3-(tosyloxy) propan-2-yl)oxy)-3,5-dichloro-2,6-dimethylphenyl)-6-(cyclopent-1-en-1-yl)thieno[2,3-d]pyrimidin-4-yl)oxy)-3-(5-((tert-butyldimethylsilyl)oxy)-2-((2-(trimethylsilyl)ethoxy)methoxy)phenyl)propanoate

To a solution of Example 125J (4.6 g) and triethylamine (2.6 mL) in dichloromethane (100 mL) was added para-toluenesulfonyl chloride (2.6 g) at 0° C., and the reaction was stirred at 25° C. for 40 hours. One additional vial was set up as described above. Both of the two mixtures were combined and poured into water and extracted with dichloromethane three times. The combined organic phases were washed with brine twice, dried over anhydrous magnesium sulfate, filtered and concentrated under reduced pressure to afford the crude product which was purified by column chromatography on silica gel (eluting with petroleum ether:ethyl acetate=97:3 to 90:10) to give the title compound. ¹H NMR (400 MHz, CDCl₃) δ ppm 8.47 (s, 1H), 7.81 (d, 2H), 7.34 (d, 2H), 6.92 (d, 1H), 6.61 (dd, 1H), 6.39 (d, 1H), 5.91 (br s, 1H), 5.82-5.67 (m, 1H), 5.35-5.27 (m, 1H), 5.21-5.06 (m, 4H), 4.67-4.57 (m, 1H), 4.51-4.37 (m, 2H), 4.14 (q, 1H), 3.94-3.79 (m, 3H), 3.78-3.66 (m, 4H), 2.62-2.49 (m, 2H), 2.46-2.37 (m, 5H), 2.23 (br t, 2H), 2.16 (s, 3H), 1.99 (s, 3H), 1.92-1.81 (m, 2H), 1.33-1.15 (m, 12H), 0.93 (s, 11H), 0.10 (d, 6H), 0.00 (s, 9H).

### Example 125L

### (R)-tert-butyl 2-((5-(4-(((R)-1-(allyloxy)-3-(tosyloxy) propan-2-yl)oxy)-3,5-dichloro-2,6-dimethylphenyl)-6-(cyclopent-1-en-1-yl)thieno[2,3-d]pyrimidin-4-yl)oxy)-3-(5-hydroxy-2-((2-(trimethylsilyl)ethoxy)methoxy)phenyl)propanoate

To a solution of Example 125K (4.6 g) in dichloromethane (46 mL) was added tetra-N-butylammonium fluoride (5.2 mL, 1M) at 0° C. After the addition, the reaction was stirred at 25° C. for 16 hours under nitrogen atmosphere. One additional vial was set up as described above. Both of the two mixtures were combined, poured into water and extracted with ethyl acetate three times. The combined organic layers were washed with brine twice, dried over anhydrous sodium sulfate, filtered and concentrated to give a residue which was purified by column chromatography on silica gel (petroleum ether:ethyl acetate=100:1 to 100:5) to give the title compound. ¹H NMR (400 MHz, CDCl₃) δ ppm 8.47 (s, 1H), 7.80 (d, 2H), 7.34 (d, 2H), 6.92 (d, 1H), 6.65 (dd, 1H), 6.02 (d, 1H), 5.89 (br s, 1H), 5.83-5.68 (m, 1H), 5.39 (dd, 1H), 5.22-5.09 (m, 5H), 4.70 (t, 1H), 4.51-4.41 (m, 2H), 3.98-3.67 (m, 7H), 2.83 (dd, 1H), 2.49-2.34 (m, 6H), 2.28-2.15 (m, 5H), 2.00-1.81 (m, 5H), 1.33 (s, 10H), 0.99-0.91 (m, 2H), 0.04-0.03 (m, 9H).

### Example 125M

### tert-butyl (7R,16R)-19,23-dichloro-1-(cyclopent-1-en-1-yl)-20,22-dimethyl-16-{[(prop-2-en-1-yl)oxy]methyl}-10-{[2-(trimethylsilyl)ethoxy]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 125L (3.6 g) in N,N-dimethylformamide (40 mL) was added cesium carbonate (5.6 g) at 0° C. and the reaction was stirred at 25° C. for 16 hours under nitrogen atmosphere. One additional vial was set up as described above. Both of the mixtures were combined, quenched with water and extracted with ethyl acetate three times. The combined organic layers were washed with brine twice, dried over anhydrous sodium sulfate, filtered and concentrated to give a residue which was purified by column chromatography on silica gel (petroleum ether:ethyl acetate=100:1 to 100:5) to give the title compound. ¹H NMR (400 MHz, CDCl₃) δ ppm 8.55 (s, 1H), 6.95 (d, 1H), 6.74 (dd, 1H), 6.03-5.90 (m, 1H), 5.87 (dd, 1H), 5.79-5.67 (m, 2H), 5.34 (qd, 1H), 5.28-5.20 (m, 1H), 5.15 (s, 2H), 5.03-4.92 (m, 1H), 4.68 (dd, 1H), 4.37-4.29 (m, 1H), 4.21-4.06 (m, 2H), 3.91-3.70 (m, 4H), 3.49 (dd, 1H), 2.87-2.77 (m, 1H), 2.35 (dt, 2H), 2.13 (s, 3H), 2.09-1.99 (m, 5H), 1.79 (m, 2H), 1.13 (s, 10H), 0.01-0.00 (m, 9H).

### Example 125N

### tert-butyl (7R,16R)-19,23-dichloro-1-(cyclopent-1-en-1-yl)-16-(hydroxymethyl)-20,22-dimethyl-10-{[2-(trimethylsilyl)ethoxy]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

To a degassed solution of Example 125M (2.3 g) in tetrahydrofuran (50 mL) and methanol (50 mL) under nitrogen atmosphere was added 1,3-dimethylpyrimidine-2,4,6,(1H,3H,5H)-trione (2.5 g) and tetrakis(triphenylphosphine)palladium (0) (2.3 g), and the reaction was stirred at 30° C. for 18 hours. One additional vial was set up as described above. Both of the mixtures were combined, poured into water and extracted with ethyl acetate three times. The combined organic phases were washed with brine twice, dried over anhydrous magnesium sulfate, filtered and concentrated under reduced pressure to afford the crude product which was purified by column chromatography on silica gel (eluting with petroleum ether:ethyl acetate=100:6 to 100:10) to give the title compound which was used in the next step directly.

### Example 1250

### tert-butyl (7R,16S)-19,23-dichloro-1-(cyclopent-1-en-1-yl)-20,22-dimethyl-16-{[(4-methylbenzene-1-sulfonyl)oxy]methyl}-10-{[2-(trimethylsilyl)ethoxy]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 125N (1.3 g) and triethylamine (1.1 mL) in dichloromethane (50 mL) was added toluenesulfonyl chloride (1.2 g) under nitrogen atmosphere at 0° C., and the reaction was stirred at 25° C. for 12 hours. Three additional vials were set up as described above. The mixtures were combined, quenched with water and extracted with ethyl acetate three times. The combined organic layers were washed with brine twice, dried over anhydrous sodium sulfate, filtered and concentrated to give a residue which was purified by column chromatography on silica gel (petroleum ether:ethyl acetate=100:1 to 100:5) to give the title compound. ¹H NMR (400 MHz, CDCl₃) δ ppm 8.55 (s, 1H), 7.86 (d, 2H), 7.37 (d, 2H), 6.96 (d, 1H), 6.69 (dd, 1H), 5.81 (dd, 1H), 5.76-5.68 (m, 2H), 5.15 (s, 2H), 5.03-4.87 (m, 1H), 4.58 (dd, 1H), 4.46-4.36 (m, 2H), 4.20 (d, 1H), 3.76 (t, 3H), 3.41 (dd, 1H), 2.84 (br d, 1H), 2.47 (s, 3H), 2.36 (br s, 2H), 2.13 (s, 3H), 1.98 (s, 5H), 1.90-1.73 (m, 3H), 1.29 (br d, 2H), 1.14 (s, 9H), 1.00-0.92 (m, 3H), 0.00 (s, 9H).

### Example 125P

### tert-butyl (7R,16R)-19,23-dichloro-1-(cyclopent-1-en-1-yl)-20,22-dimethyl-16-[(4-methylpiperazin-1-yl)methyl]-10-{[2-(trimethylsilyl)ethoxy]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 1250 (1.6 g) in N,N-dimethylformamide (16 mL) was added 1-methylpiperazine (16 mL) under nitrogen atmosphere at 0° C., and the reaction was stirred at 55° C. for 12 hours. Two other vials were set up as described above. The three reaction mixtures were combined and concentrated to a residue. The residue was dissolved in ethyl acetate and washed with brine twice. The organic phase was dried over anhydrous magnesium sulfate, filtered and concentrated to give the crude product. The crude product was purified by column chromatography on silica gel (eluting with petroleum ether:ethyl acetate=1:1) to provide the title compound.

### Example 125Q

### tert-butyl (7R,16R)-19,23-dichloro-1-(cyclopent-1-en-1-yl)-10-hydroxy-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 125P (2.1 g) in dichloromethane (75 mL) was added HCl (1.1 mL, 1M in methanol) under nitrogen atmosphere at 0° C., and the reaction was stirred at 25° C. for 2 hours. Two additional vials were set up as described above. The three reaction mixtures were combined, quenched with saturated aqueous sodium bicarbonate solution at 0° C. and extracted with ethyl acetate three times. The combined organic layers were washed with brine twice, dried over anhydrous sodium sulfate, filtered and concentrated to give the title compound. ¹H NMR (400 MHz, CDCl₃) δ ppm 8.57 (s, 1H), 6.76-6.61 (m, 2H), 5.94 (dd, 1H), 5.73 (br s, 1H), 5.64 (d, 1H), 4.89 (q, 1H), 4.67-4.52 (m, 1H), 4.31 (br d, 1H), 3.66-3.49 (m, 1H), 2.91 (dd, 1H), 2.83-2.67 (m, 3H), 2.66-2.43 (m, 6H), 2.43-2.27 (m, 5H), 2.17-1.99 (m, 8H), 1.81 (m, 2H), 1.11 (s, 9H).

### Example 125R

### tert-butyl (7R,16R)-19,23-dichloro-1-(cyclopent-1-en-1-yl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 16N with Example 125Q. MS (ESI) m/z 1147.6 (M+H)⁺.

### Example 125S

### (7R,16R)-19,23-dichloro-1-(cyclopent-1-en-1-yl)-10-({2-[(4S*)-4-fluoro-4-(2,5,8,11-tetraoxadodecan-1-yl)cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 125R. ¹H NMR (400 MHz, dimethylsulfoxide-d₆) δ ppm 8.73 (d, 1H), 8.63 (s, 1H), 7.42 (d, 1H), 7.14 (d, 1H), 6.90-6.65 (m, 2H), 6.16 (dd, 1H), 5.87 (d, 1H), 5.76 (p, 1H), 5.21-5.01 (m, 2H), 4.89 (q, 1H), 4.48 (d, 2H), 3.64-3.49 (m, 14H), 3.42 (dd, 3H), 3.23 (s, 3H), 2.87 (dd, 1H), 2.69 (dd, 2H), 2.42-2.28 (m, 6H), 2.19 (s, 3H), 2.04 (s, 4H), 1.91 (s, 5H), 1.75 (q, 2H). MS (ESI) m/z 1091.5 (M+H)⁺.

### Example 126

### (7R,16R)-19,23-dichloro-10-[(2-{4-[(1,1-dioxo-1˜⁶-thiomorpholin-4-yl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 126A

### 4-(4-(4-(hydroxymethyl)pyrimidin-2-yl)benzyl)thiomorpholine 1,1-dioxide

The title compound was prepared by substituting 4-(4-(4,4,5,5-tetramethyl-1,3,2-dioxaborolan-2-yl)benzyl)thiomorpholine 1,1-dioxide for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.87 (d, 1H), 8.35 (d, 2H), 7.49-7.46 (m, 3H), 5.67 (t, 1H), 4.63 (d, 2H), 3.75 (s, 2H), 3.11 (m, 4H), 2.91 (m, 4H). MS (ESI) m/z 334.2 (M+H)⁺.

### Example 126B

### (7R,16R)-19,23-dichloro-10-[(2-{4-[(1,1-dioxo-1λ⁶-thiomorpholin-4-yl)methyl]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 126A for Example 38D in Example 38E. ¹H NMR (500 MHz, dimethylsulfoxide-d₆) δ ppm 8.81 (d, 1H), 8.67 (s, 1H), 8.30 (d, 2H), 7.45 (d, 1H), 7.42 (d, 2H), 7.15-7.05 (m, 4H), 6.82 (d, 1H), 6.68 (dd, 1H), 6.18 (m, 1H), 5.76 (s, 1H), 5.16 (q, 2H), 4.79 (m, 1H), 4.38 (m, 2H), 3.68 (s, 2H), 3.59 (dd, 1H), 3.06 (m, 4H), 2.92 (d, 2H), 2.84 (m, 4H), 2.60 (m, 3H), 2.35 (m, 6H), 2.12 (s, 3H), 1.91 (s, 3H), 1.89 (s, 3H). MS (ESI) m/z 1068.4 (M+H)⁺.

### Example 127

### (7R,16R)-19,23-dichloro-1-(4-fluorophenyl)-10-({2-[(4R)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 127A

### 8-((2-(2-methoxyethoxy)ethoxy)methyl)-1,4-dioxaspiro[4.5]decane

Example 127A was synthesized according to the procedure described for Example 72E, substituting 1,4-dioxaspiro[4.5]decan-8-ylmethanol for Example 72C and also substituting 1-bromo-2-(2-methoxyethoxy) ethane for Example 72D. MS (APCI) m/z 275.4 (M+H)⁺.

### Example 127B

### 4-((2-(2-methoxyethoxy)ethoxy)methyl)cyclohexanone

To a solution of Example 127A (2.9 g) in tetrahydrofuran (30 mL) was added a 6 molar aqueous solution of HCl (30 mL) and the reaction was stirred at room temperature overnight. The mixture was poured into a 500 mL separatory funnel and diluted with 250 ml of water. The aqueous layer was extracted with three portions of ethyl acetate. The organic layers were combined and dried over anhydrous magnesium sulfate, filtered and concentrated onto silica gel. Purification by flash chromatography on a CombiFlash® Teledyne Isco system using a Teledyne Isco RediSep® Rf gold 120 g silica gel column (eluting with solvent A=2:1 ethyl acetate:ethanol; solvent B=Heptane, 10-70% A to B) afforded the title compound. MS (APCI) m/z 231.5 (M+H)⁺.

### Example 127C

### (R)-((4-((2-(2-methoxyethoxy)ethoxy)methyl)cyclohex-1-en-1-yl)oxy)trimethylsilane

A solution of (S)-bis((S)-1-phenylethyl)amine (769 mg) in tetrahydrofuran was cooled to −78° C. and stirred under nitrogen before N-butyllithium (1.3 mL) was added dropwise over 10 minutes. Stirring was continued at −78° C. for 30 minutes and trimethylchlorosilane (1.7 mL) was added dropwise over 10 minutes. After an additional 10 minutes of stirring, a solution of Example 127B (600 mg) in 1.3 mL of tetrahydrofuran was added dropwise over 30 minutes. The mixture was stirred at −78° C. for 20 minutes longer then treated with triethylamine (3.7 mL) and stirred an extra 15 minutes. The cooling bath was removed and saturated aqueous sodium bicarbonate (10 mL) was added. The mixture was allowed to warm to ambient temperature and poured into a separatory funnel containing water and diethyl ether. The mixture was partitioned between the two phases, the organic layer was removed and the aqueous layer was washed with one more portion of diethyl ether. The organic layers were combined, dried over anhydrous magnesium sulfate, filtered and concentrated onto silica gel. Purification by flash chromatography on a CombiFlash® Teledyne Isco system using a Teledyne Isco RediSep® Rf gold 40 g silica gel column (eluting 0-40% ethyl acetate/heptane) afforded the title compound. ¹H NMR (400 MHz, dimethylsulfoxide-d₆) δ ppm 4.80-4.70 (m, 1H), 3.55-3.45 (m, 6H), 3.45-3.40 (m, 2H), 3.30-3.25 (m, 2H), 3.24 (s, 3H), 2.10-1.95 (m, 2H), 1.95-1.82 (m, 1H), 1.81-1.62 (m, 3H), 1.37-1.21 (m, 1H), 0.14 (s, 9H).

### Example 127D

### (R)-4-((2-(2-methoxyethoxy)ethoxy)methyl)cyclohex-1-en-1-yl trifluoromethanesulfonate

To a stirring solution of Example 127C (500 mg) in tetrahydrofuran (6.0 mL), at 0° C. under nitrogen, was added 2.1 mL of methyllithium and stirring was continued for 30 minutes. TMEDA (N,N,N′,N′-tetramethylethylenediamine, 1.3 mL) was added followed by a solution of N,N-bis(trifluoromethylsulfonyl) aniline (768 mg) in 6 mL of tetrahydrofuran. The reaction mixture was stirred at 0° C. for 1 hour and allowed to warm to room temperature. The mixture was quenched with saturated aqueous sodium bicarbonate and the aqueous layer was extracted with two portions of ethyl acetate. The organic layers were combined then dried over anhydrous magnesium sulfate, filtered and concentrated onto silica gel. Purification by flash chromatography on a CombiFlash® Teledyne Isco system using a Teledyne Isco RediSep® Rf gold 40 g silica gel column (eluting 10-100% ethyl acetate/heptane) afforded the title compound. MS (APCI) m/z 363.3 (M+H)⁺.

### Example 127E

### (R)-2-(4-((2-(2-methoxyethoxy)ethoxy)methyl)cyclohex-1-en-1-yl)-4,4,5,5-tetramethyl-1,3,2-dioxaborolane

A 20 mL reaction vessel, equipped with stir bar, was charged with Example 127D (530 mg), bis(pinacolato)diboron (483 mg), [1,1′-bis(diphenylphosphino)ferrocene]dichloropalladium (II) (107 mg) and potassium acetate (287 mg). The flask was capped then evacuated and backfilled with nitrogen twice. Dioxane (12 mL) was added via syringe and the stirring mixture was evacuated and backfilled with nitrogen twice again. The mixture stirred at 80° C. overnight. After cooling to ambient temperature, the mixture was filtered through a diatomaceous earth pad, the filter cake washed with ethyl acetate and the filtrate was concentrated onto silica gel. Purification by flash chromatography on a CombiFlash® Teledyne Isco system using a Teledyne Isco RediSep® Rf gold 40 g silica gel column (eluting 10-80% ethyl acetate/heptane) afforded the title compound. ¹H NMR (400 MHz, dimethylsulfoxide-d₆) δ ppm 6.52-6.35 (m, 1H), 3.54-3.45 (m, 6H), 3.44-3.40 (m, 2H), 3.26 (dd, J=6.2, 1.9 Hz, 2H), 3.24 (s, 3H), 2.22-2.03 (m, 2H), 2.02-1.88 (m, 1H), 1.80-1.65 (m, 3H), 1.18 (s, 12H), 1.15-1.08 (m, 1H).

### Example 127F

### (R)-4-(((tert-butyldimethylsilyl)oxy)methyl)-2-(4-((2-(2-methoxyethoxy)ethoxy)methyl)cyclohex-1-en-1-yl)pyrimidine

An 8 mL reaction vessel, equipped with stir bar, was charged with Example 38B (100 mg), Example 127E (158 mg), [1,1′-bis(diphenylphosphino)ferrocene]dichloropalladium (II) (28 mg) and potassium phosphate (205 mg). The flask was capped with a septa and evacuated and backfilled with nitrogen twice before dioxane (2.1 mL) and water (0.5 mL) were added. The stirring mixture was evacuated, backfilled with nitrogen twice, and stirred at 80° C. for 16 hours. After cooling to ambient temperature, the reaction was poured into a separatory funnel containing water and brine and extracted three times with ethyl acetate. The organic layers were combined and concentrated onto silica gel. Purification by flash chromatography on a CombiFlash® Teledyne Isco system using a Teledyne Isco RediSep® Rf gold 12 g silica gel column (eluting 10-100% ethyl acetate/heptane) afforded the title compound. MS (APCI) m/z 437.4 (M+H)⁺.

### Example 127G

### (R)-(2-(4-((2-(2-methoxyethoxy)ethoxy)methyl)cyclohex-1-en-1-yl)pyrimidin-4-yl)methanol

Example 127G was synthesized according to the procedure described for Example 72C, substituting Example 127F for Example 72B. MS (APCI) m/z 323.4 (M+H)⁺.

### Example 127H

### tert-butyl (7R,16R)-19,23-dichloro-1-(4-fluorophenyl)-10-({2-[(4R)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 127H was synthesized according to the procedure described for Example 291, substituting Example 127G for Example 29H. MS (APCI) m/z 1114.8 (M+H)⁺.

### Example 127I

### (7R,16R)-19,23-dichloro-1-(4-fluorophenyl)-10-({2-[(4R)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 127I was synthesized according to the procedure described for Example 29J, substituting Example 127H for Example 291. ¹H NMR (500 MHz, dimethylsulfoxide-d₆) δ ppm 8.73 (s, 1H), 8.71 (d, 1H), 7.37 (d, 1H), 7.26-7.22 (m, 1H), 7.22-7.16 (m, 2H), 7.15-7.10 (m, 2H), 6.83 (d, 1H), 6.73 (dd, 1H), 6.23 (dd, 1H), 5.80 (d, 1H), 5.14 (d, 1H), 5.07 (d, 1H), 4.90-4.80 (m, 1H), 4.44 (d, 2H), 3.66-3.59 (m, 1H), 3.54-3.49 (m, 6H), 3.44-3.41 (m, 4H), 3.35-3.31 (m, 2H), 3.23 (s, 3H), 3.00-2.89 (m, 1H), 2.76-2.60 (m, 3H), 2.48-2.30 (m, 10H), 2.24 (s, 3H), 1.98 (s, 3H), 1.95 (s, 3H), 1.93-1.77 (m, 2H), 1.36-1.25 (m, 1H). MS (APCI) m/z 1059.0 (M+H)⁺.

### Example 128

### (7R,16R)-19,23-dichloro-1-(4-fluorophenyl)-10-({2-[(4S)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 128A

### (S)((4-((2-(2-methoxyethoxy)ethoxy)methyl)cyclohex-1-en-1-yl)oxy)trimethylsilane

Example 128A was synthesized according to the procedure described for Example 127C, substituting (R)-bis((R)-1-phenylethyl)amine for (S)-bis((S)-1-phenylethyl)amine. ¹H NMR (400 MHz, dimethylsulfoxide-d₆) δ ppm 4.82-4.67 (m, 1H), 3.58-3.45 (m, 6H), 3.45-3.38 (m, 2H), 3.30-3.25 (m, 2H), 3.24 (s, 3H), 2.10-1.95 (m, 2H), 1.95-1.84 (m, 1H), 1.79-1.60 (m, 3H), 1.34-1.22 (m, 1H), 0.14 (d, 9H).

### Example 128B

### (S)-4-((2-(2-methoxyethoxy)ethoxy)methyl)cyclohex-1-en-1-yl trifluoromethanesulfonate

Example 128B was synthesized according to the procedure described for Example 127D, substituting Example 128A for Example 127C. MS (APCI) m/z 363.3 (M+H)⁺.

### Example 128C

### (S)-2-(4-((2-(2-methoxyethoxy)ethoxy)methyl)cyclohex-1-en-1-yl)-4,4,5,5-tetramethyl-1,3,2-dioxaborolane

Example 128C was synthesized according to the procedure described for Example 127E, substituting Example 128B for Example 127D. ¹H NMR (400 MHz, dimethylsulfoxide-d₆) δ ppm 6.52-6.35 (m, 1H), 3.54-3.45 (m, 6H), 3.44-3.40 (m, 2H), 3.26 (dd, 2H), 3.24 (s, 3H), 2.22-2.03 (m, 2H), 2.02-1.88 (m, 1H), 1.80-1.65 (m, 3H), 1.18 (s, 12H), 1.15-1.08 (m, 1H).

### Example 128D

### (S)-4-(((tert-butyldimethylsilyl)oxy)methyl)-2-(4-((2-(2-methoxyethoxy)ethoxy)methyl)cyclohex-1-en-1-yl)pyrimidine

Example 128D was synthesized according to the procedure described for Example 127F, substituting Example 128C for Example 127E. MS (APCI) m/z 437.4 (M+H)⁺.

### Example 128E

### (S)-(2-(4-((2-(2-methoxyethoxy)ethoxy)methyl)cyclohex-1-en-1-yl)pyrimidin-4-yl)methanol

Example 128E was synthesized according to the procedure described for Example 72C, substituting Example 128D for Example 72B. MS (APCI) m/z 323.4 (M+H)⁺.

### Example 128F

### tert-butyl (7R,16R)-19,23-dichloro-1-(4-fluorophenyl)-10-({2-[(4S)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 128F was synthesized according to the procedure described for Example 29J, substituting Example 128E for Example 29H. MS (APCI) m/z 1114.8 (M+H)⁺.

### Example 128G

### (7R,16R)-19,23-dichloro-1-(4-fluorophenyl)-10-({2-[(45)-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 128G was synthesized according to the procedure described for Example 29J, substituting Example 128F for Example 291. ¹H NMR (500 MHz, dimethylsulfoxide-d₆) δ ppm 8.73 (s, 1H), 8.71 (d, 1H), 7.37 (d, 1H), 7.26-7.22 (m, 1H), 7.22-7.16 (m, 2H), 7.15-7.10 (m, 2H), 6.83 (d, 1H), 6.73 (dd, 1H), 6.23 (dd, 1H), 5.80 (d1H), 5.14 (d, 1H), 5.07 (d, 1H), 4.90-4.80 (m, 1H), 4.44 (d, 2H), 3.66-3.59 (m, 1H), 3.54-3.49 (m, 6H), 3.44-3.41 (m, 4H), 3.35-3.31 (m, 2H), 3.23 (s, 3H), 3.00-2.89 (m, 1H), 2.76-2.60 (m, 3H), 2.48-2.30 (m, 10H), 2.24 (s, 3H), 1.98 (s, 3H), 1.95 (s, 3H), 1.93-1.77 (m, 2H), 1.36-1.25 (m, 1H). MS (APCI) m/z 1059.0 (M+H)⁺.

### Example 129

### (7R,16R)-19,23-dichloro-1-(4-fluorophenyl)-20,22-dimethyl-16-[(4-methylpiperazin-1-yl)methyl]-10-[(2-{4-[(1,4,7,10-tetraoxa-13-azacyclopentadecan-13-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 129A

### 13-(4-(4,4,5,5-tetramethyl-1,3,2-dioxaborolan-2-yl)benzyl)-1,4,7,10-tetraoxa-13-azacyclopentadecane

1,4,7,10-Tetraoxa-13-azacyclopentadecane (255 mg) and 2-(4-(bromomethyl)phenyl)-4,4,5,5-tetramethyl-1,3,2-dioxaborolane (300 mg) were dissolved in tetrahydrofuran (5 mL). Triethylamine (307 mg) was added, and the solution was stirred at room temperature for 15 minutes. The solution was filtered and concentrated under vacuum. The material was purified by flash column chromatography on silica gel using a gradient of 0-10% methanol in dichloromethane. The solvent was removed under vacuum to yield the title compound. ¹H NMR (500 MHz, dimethylsulfoxide-d₆) δ ppm 7.61 (d, 2H), 7.31 (d, 2H), 3.63 (s, 2H), 3.56-3.48 (m, 16H), 2.63 (t, 4H), 1.29 (s, 12H). MS (ESI) m/z 436.3 (M+H)⁺.

### Example 129B

### (2-(4-((1,4,7,10-tetraoxa-13-azacyclopentadecan-13-yl)methyl)phenyl)pyrimidin-4-yl)methanol

The title compound was prepared by substituting Example 129A for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.87 (d, 1H), 8.32 (d, 2H), 7.49-7.44 (m, 3H), 5.67 (t, 1H), 4.63 (d, 2H), 3.70 (s, 2H), 3.58-3.51 (m, 16H), 2.68 (m, 4H). MS (ESI) m/z 418.4 (M+H)⁺.

### Example 129C

### (7R,16R)-19,23-dichloro-1-(4-fluorophenyl)-20,22-dimethyl-16-[(4-methylpiperazin-1-yl)methyl]-10-[(2-{4-[(1,4,7,10-tetraoxa-13-azacyclopentadecan-13-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 129B for Example 38D in Example 38E. ¹H NMR (500 MHz, dimethylsulfoxide-d₆) δ ppm 8.88 (d, 1H), 8.76 (s, 1H), 8.34 (d, 2H), 7.50 (d, 1H), 7.47 (d, 2H), 7.22-7.13 (m, 4H), 6.91 (d, 1H), 6.79 (dd, 1H), 6.28 (m, 1H), 5.79 (d, 1H), 5.24 (q, 2H), 4.87 (m, 1H), 4.45 (m, 2H), 3.72 (s, 2H), 3.68 (dd, 2H), 3.58-3.51 (m, 18H), 3.01 (dd, 2H), 2.76-2.68 (m, 11H), 2.41 (s, 3H), 1.99 (s, 3H), 1.95 (s, 3H). MS (ESI) m/z 1152.5 (M+H)⁺.

### Example 130

### (7R,16R)-19,23-dichloro-1-cyclobutyl-10-({2-[(4R*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 130A

### 4-chloro-5-(3,5-dichloro-4-methoxy-2,6-dimethylphenyl)thieno[2,3-d]pyrimidine

To a suspension of Example 15E (4 g) in acetonitrile (50 mL) was added N-chlorosuccinimide (3.86 g) and tetrafluoroboric acid diethyl ether complex (4.68 g). The reaction mixture was stirred at 15° C. under nitrogen for 16 hours. The reaction mixture was diluted with water (30 mL) and extracted three times with ethyl acetate (200 mL). The organic layer was dried over Na₂SO₄, filtered and concentrated. The residue was purified by column chromatography on silica gel (petroleum ether:ethyl acetate from 200:1 to 20:1) to provide the title compound. ¹H NMR (400 MHz, dimethylsulfoxide-d₆) δ ppm 9.01 (s, 1H), 8.02 (s, 1H), 3.88 (s, 3H), 2.01 (s, 6H).

### Example 130B

### 6-bromo-4-chloro-5-(3,5-dichloro-4-methoxy-2,6-dimethylphenyl)thieno[2,3-d]pyrimidine

To a solution of Example 130A (3.0 g) in tetrahydrofuran (50 mL) cooled to −78° C., was added lithium diisopropylamide (2M in tetrahydrofuran/heptane/ethylbenzene, 6.02 mL) and the mixture was stirred at −78° C. for 90 minutes. 1,2-Dibromotetrachloroethane (3.14 g) was added in three portions over 10 minutes and stirring was continued at −78° C. for 1 hour. The mixture was allowed to warm to −30° C. water (60 mL) was added, and the mixture was extracted twice with ethyl acetate (40 mL). The combined organic extracts washed with brine, dried over magnesium sulfate, filtered and concentrated. Purification by chromatography on silica gel using an ISCO CombiFlash® Companion MPLC (10 g Chromabond® column, eluting with 0-20% heptane/ethyl acetate) provided the title compound. ¹H NMR (600 MHz, dimethylsulfoxide-d₆) δ ppm 10.22 (s, 1H), 9.00 (s, 1H), 1.96 (s, 6H). MS (ESI) m/z 450.95 (M+H)⁺.

### Example 130C

### 4-(6-bromo-4-chlorothieno[2,3-d]pyrimidin-5-yl)-2,6-dichloro-3,5-dimethylphenol

To a solution of Example 130B (4.35 g) in 1,2-dichloroethane (60 mL) at 15° C. was added AlCl₃ (3.84 g) in three portions over 5 minutes, and the mixture was stirred for 10 minutes at ambient temperature. Boron trichloride (1 M in dichloromethane-24.03 mL) was added dropwise over 5 minutes, and the mixture was stirred for 2 hours. The mixture was allowed to warm to 5° C., and water (50 mL) was added. The mixture was extracted twice with dichloromethane (40 mL), and the combined organic extracts were washed twice with HCl (1 M aqueous solution-30 mL), dried over magnesium sulfate, filtered, and concentrated to provide the title compound. MS (ESI) m/z 436.8 (M+H)⁺.

### Example 130D

### (R)-5-(4-((1-(allyloxy)-3-(bis(4-methoxyphenyl)(phenyl)methoxy) propan-2-yl)oxy)-3,5-dichloro-2,6-dimethylphenyl)-6-bromo-4-chlorothieno[2,3-d]pyrimidine

The title compound was prepared as described in Example 15K by substituting Example 130C for Example 151. ¹H NMR (400 MHz, chloroform-d) δ ppm 8.85 (s, 1H), 7.47-7.41 (m, 2H), 7.36-7.30 (m, 5H), 7.30-7.24 (m, 3H), 7.23-7.15 (m, 1H), 5.82 (ddt, 1H), 5.19 (dq, 1H), 5.11 (dq, 1H), 4.74 (p, 1H), 3.97 (dt, 2H), 3.86-3.81 (m, 2H), 3.79 (s, 6H), 3.59-3.49 (m, 2H), 2.01 (s, 3H), 2.01 (s, 3H). MS (ESI) m/z 877.0 [M+H]⁺.

### Example 130E

### (R)-tert-butyl 2-((5-(4-(((R)-1-(allyloxy)-3-(bis(4-methoxyphenyl) (phenyl)methoxy) propan-2-yl)oxy)-3,5-dichloro-2,6-dimethylphenyl)-6-bromothieno[2,3-d]pyrimidin-4-yl)oxy)-3-(2-(benzyloxy)-5-((tert-butyldimethylsilyl)oxy)phenyl)propanoate

The title compound was prepared as described in Example 16F by substituting Example 130D for Example 15K. ¹H NMR (501 MHz, chloroform-d) δ ppm 8.51 (s, 1H), 7.46-7.39 (m, 2H), 7.39-7.32 (m, 2H), 7.35-7.28 (m, 4H), 7.28-7.22 (m, 2H), 7.22-7.15 (m, 1H), 6.83-6.75 (m, 4H), 6.69 (d, 1H), 6.60 (dd, 1H), 6.40 (d, 1H), 5.77 (ddt, 1H), 5.39 (t, 1H), 5.13 (dq, 1H), 5.07 (dq, 1H), 4.98 (d, 1H), 4.94 (d, 1H), 4.60 (p, 1H), 3.90 (ddt, 2H), 3.78 (s, 6H), 3.83-3.72 (m, 2H), 3.59-3.50 (m, 2H), 2.67 (d, 2H), 2.13 (s, 3H), 1.93 (s, 3H), 1.31 (s, 1H), 1.35-1.23 (m, 1H), 1.28 (s, 2H), 1.26 (s, 9H), 0.93 (s, 9H), 0.10 (s, 3H), 0.09 (s, 3H). MS (ESI) m/z 1275 [M+H]⁺.

### Example 130F

### (R)-tert-butyl 2-((5-(4-(((S)-1-(allyloxy)-3-hydroxypropan-2-yl)oxy)-3,5-dichloro-2,6-dimethylphenyl)-6-bromothieno[2,3-d]pyrimidin-4-yl)oxy)-3-(2-(benzyloxy)-5-((tert-butyldimethylsilyl)oxy)phenyl)propanoate

The title compound was prepared as described in Example 16G by substituting Example 130E for Example 16F. ¹H NMR (400 MHz, chloroform-d) δ ppm 8.47 (d, 1H), 7.39-7.31 (m, 2H), 7.31-7.23 (m, 2H), 7.27-7.17 (m, 1H), 6.68 (d, 1H), 6.57 (dd, 1H), 6.35 (d, 1H), 5.78 (ddt, 1H), 5.39 (t, 1H), 5.16 (dt, 1H), 5.08 (dd, 1H), 4.96 (d, 1H), 4.92 (d, 1H), 4.53-4.44 (m, 1H), 3.91 (dddd, 3H), 3.81 (ddd, 1H), 3.79-3.70 (m, 2H), 2.66 (dd, 1H), 2.58 (dd, 1H), 2.31 (dd, 1H), 2.09 (s, 3H), 1.91 (s, 3H), 1.22 (s, 9H), 0.88 (s, 9H), 0.06 (s, 3H), 0.05 (s, 3H). MS (DCI) m/z 973.2 [M+H]⁺.

### Example 130G

### (R)-tert-butyl 2-((5-(4-(((R)-1-(allyloxy)-3-(tosyloxy) propan-2-yl)oxy)-3,5-dichloro-2,6-dimethylphenyl)-6-bromothieno[2,3-d]pyrimidin-4-yl)oxy)-3-(2-(benzyloxy)-5-((tert-butyldimethylsilyl)oxy)phenyl)propanoate

The title compound was prepared as described in Example 16H substituting Example 130F for Example 16G. ¹H NMR (400 MHz, chloroform-d) δ ppm 8.46 (s, 1H), 7.77-7.68 (m, 2H), 7.36-7.28 (m, 2H), 7.28-7.17 (m, 5H), 6.66 (d, 1H), 6.56 (dd, 1H), 6.34 (d, 1H), 5.75-5.61 (m, 1H), 5.35 (t, 1H), 5.13-5.00 (m, 2H), 4.95 (d, 1H), 4.91 (d, 1H), 4.51 (p, 1H), 4.41 (dd, 1H), 4.33 (dd, 1H), 3.87-3.73 (m, 2H), 3.66 (dd, 1H), 3.61 (dd, 1H), 2.64 (dd, 1H), 2.57 (dd, 1H), 2.38 (s, 3H), 2.06 (s, 3H), 1.87 (s, 3H), 1.22 (s, 9H), 0.88 (s, 9H), 0.06 (s, 3H). MS (ESI) m/z 1127.3 [M+H]⁺.

### Example 130H

### (R)-tert-butyl 2-((5-(4-(((R)-1-(allyloxy)-3-(tosyloxy) propan-2-yl)oxy)-3,5-dichloro-2,6-dimethylphenyl)-6-bromothieno[2,3-d]pyrimidin-4-yl)oxy)-3-(2-(benzyloxy)-5-hydroxyphenyl)propanoate

The title compound was prepared as described in Example 161 substituting Example 130G for Example 16H. ¹H NMR (501 MHz, chloroform-d) δ ppm 8.51 (s, 1H), 7.82-7.75 (m, 2H), 7.44-7.38 (m, 2H), 7.37-7.29 (m, 4H), 7.32-7.25 (m, 1H), 6.73 (d, 1H), 6.64 (dd, 1H), 5.96 (d, 1H), 5.76 (ddt, 1H), 5.52 (dd, 1H), 5.16 (dq, 1H), 5.12 (dt, 1H), 5.01 (s, 1H), 4.99 (s, 2H), 4.69-4.61 (m, 1H), 4.48 (dd, 1H), 4.41 (dd, 1H), 3.97-3.82 (m, 2H), 3.78 (dd, 1H), 3.74 (dd, 1H), 2.99 (dd, 1H), 2.43 (s, 3H), 2.39 (dd, 1H), 2.18 (s, 3H), 1.97 (s, 3H), 1.31 (s, 9H). MS (ESI) m/z 1112.8 [M+H]⁺.

### Example 1301

### tert-butyl (7R,16R)-10-(benzyloxy)-1-bromo-19,23-dichloro-20,22-dimethyl-16-{[(prop-2-en-1-yl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 16J substituting Example 130H for Example 161. ¹H NMR (400 MHz, chloroform-d) δ ppm 8.59 (s, 1H), 7.47-7.40 (m, 2H), 7.42-7.34 (m, 2H), 7.37-7.28 (m, 1H), 6.80-6.70 (m, 2H), 6.03-5.88 (m, 2H), 5.82 (d, 1H), 5.35 (dq, 1H), 5.24 (dq, 1H), 5.09-5.01 (m, 1H), 5.04-4.94 (m, 2H), 4.63 (dd, 1H), 4.35 (dd, 1H), 4.23-4.07 (m, 2H), 3.91 (dd, 1H), 3.82 (dd, 1H), 3.48 (dd, 1H), 2.91 (dd, 1H), 2.19 (s, 3H), 1.98 (s, 3H), 1.20 (s, 9H). MS (ESI) m/z 841.1 [M+H]⁺.

### Example 130J

### tert-butyl (7R,16R)-10-(benzyloxy)-1-bromo-19,23-dichloro-16-(hydroxymethyl)-20,22-dimethyl-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 16K substituting Example 1301 for Example 16J. ¹H NMR (400 MHz, chloroform-d) δ ppm 8.57 (s, 1H), 7.46-7.40 (m, 2H), 7.37 (ddd, 2H), 7.35-7.26 (m, 1H), 6.75 (d, 1H), 6.71 (dd, 1H), 5.86 (dd, 1H), 5.82 (d, 1H), 5.12 (dddd, 1H), 5.01 (d, 1H), 4.97 (d, 1H), 4.61 (dd, 1H), 4.23 (dd, 1H), 4.06 (ddd, 1H), 3.93 (ddd, 1H), 3.35 (dd, 1H), 2.98 (dd, 1H), 2.34 (dd, 1H), 2.21 (s, 3H), 1.95 (s, 3H), 1.22 (s, 9H). MS (ESI) m/z 801.0 [M+H]⁺.

### Example 130K

### tert-butyl (7R,16S)-10-(benzyloxy)-1-bromo-19,23-dichloro-20,22-dimethyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 16L substituting Example 130J for Example 16K. ¹H NMR (501 MHz, Chloroform-d) δ 8.57 (s, 1H), 7.89-7.83 (m, 2H), 7.45-7.40 (m, 2H), 7.40-7.33 (m, 4H), 7.35-7.28 (m, 1H), 6.76 (d, 1H), 6.69 (dd, 1H), 5.86 (dd, 1H), 5.77 (d, 1H), 5.09-4.98 (m, 2H), 4.98 (d, 1H), 4.52 (dd, 1H), 4.43 (dd, 1H), 4.37 (dd, 1H), 4.22 (dd, 1H), 3.38 (dd, 1H), 2.93 (dd, 1H), 2.45 (s, 3H), 2.17 (s, 3H), 1.92 (s, 3H), 1.20 (s, 9H). MS (ESI) m/z 955.0 [M+H]⁺.

### Example 130L

### tert-butyl (7R,16S)-10-(benzyloxy)-1-bromo-19,23-dichloro-20,22-dimethyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 16M substituting Example 130K for Example 16L. ¹H NMR (400 MHz, dimethylsulfoxide-d₆) δ ppm 8.68 (s, 1H), 7.41-7.35 (m, 2H), 7.35-7.28 (m, 2H), 7.31-7.22 (m, 1H), 6.87 (d, 1H), 6.79 (dd, 1H), 5.97 (dd, 1H), 5.59 (d, 1H), 5.01 (d, 1H), 4.93 (d, 1H), 4.70 (tt, 1H), 4.51-4.38 (m, 2H), 3.58-3.49 (m, 1H), 2.78-2.65 (m, 1H), 2.66 (d, 2H), 2.41 (s, 4H), 2.28 (s, 4H), 2.11 (s, 3H), 1.98 (s, 3H), 1.93 (s, 3H), 1.03 (s, 9H). MS (ESI) m/z 883.4 [M+H]⁺.

### Example 130M

### tert-butyl (4R,9R)-66-(benzyloxy)-13,15-dichloro-26-cyclobutyl-12,16-dimethyl-9-((4-methylpiperazin-1-yl)methyl)-3,7,10-trioxa-2(5,4)-thieno[2,3-d]pyrimidina-1(1,4), 6(1,3)-dibenzenacyclodecaphane-4-carboxylate

To a 5 mL microwave vial, which was dried for 24 hours at 70° C. under vacuum and stored in a glove box, was added Example 130L (200 mg), potassium cyclobutyltrifluoroborate (80 mg), Cs₂CO₃ (150 mg), [Ni(dtbbpy)]Cl₂ (9 mg), and Ir[dF(CF₃)ppy]₂(dtbbpy) (25 mg) in a glove box. Freshly degassed dioxane (1 mL) was added and the reaction mixture exposed to blue light (34W Blue LED KESSIL Light, EvoluChem™ PhotoRedOx Box) under stirring at 25° C. for 20 hours. The reaction mixture was concentrated, water (20 mL) was added and the mixture was extracted twice with ethyl acetate (10 mL). The combined organic extracts were washed with brine, dried over magnesium sulfate, filtered and concentrated in vacuo. Purification by chromatography on silica gel using an ISCO CombiFlash® Companion MPLC (4 g Chromabond® SiOH column, eluting with 0-10% dichloromethane/methanol) and subsequent purification by SFC (Viridis PFP 250×19 mm 5 μm column; gradient 5-50% liquid CO₂ in methanol+0.2% ammonium hydroxide) provided the title compound. MS (APCI) m/z 859.3 (M+H)⁺.

### Example 130N

### tert-butyl (4R,9R)-13,15-dichloro-26-cyclobutyl-66-hydroxy-12,16-dimethyl-9-((4-methylpiperazin-1-yl)methyl)-3,7,10-trioxa-2 (5,4)-thieno[2,3-d]pyrimidina-1(1,4),6(1,3)-dibenzenacyclodecaphane-4-carboxylate

A Tinyclave steel reactor (Buechi) was charged with Example 130M (165 mg) in tetrahydrofuran (10 mL) and Pd/C (50% wet with water, 50 mg) was added. The reactor was purged with hydrogen gas three times, stirred under hydrogen first with a pressure of 50 psi for 24 hours and under a pressure of 100 psi for 96 hours. The reaction was vented, the mixture was filtered through diatomaceous earth, and the filtrate was concentrated in vacuo. Purification by chromatography on silica gel using an ISCO CombiFlash® Companion MPLC (4 g Chromabond® SiOH column, eluting with 0-10% dichloromethane/methanol) provided the title compound. MS (APCI) m/z 769.3 (M+H)⁺.

### Example 1300

### 8-(bromomethyl)-8-fluoro-1,4-dioxaspiro[4.5]decane

To a mixture of 8-methylene-1,4-dioxaspiro[4.5]decane (30 g) and 1-bromopyrrolidine-2,5-dione (41.6 g) in dichloromethane was added triethylamine trihydrofluoride (47.0 g) at 0° C. After 15 minutes, stirring was continued at 20° C. for 2 hours. The mixture was poured into ice-water, neutralized with saturated aqueous sodium bicarbonate to pH 8 and extracted with dichloromethane twice. The combined extracts were washed with 0.1N aqueous HCl and with 5% aqueous sodium bicarbonate solution, dried with sodium sulfate, filtered and concentrated. The crude product was purified by column chromatography (n-hexane/ethyl acetate=3:1) to provide the title compound. ¹H NMR (400 MHz, CDCl₃) δ ppm 3.97 (m, 4H), 3.49 (d, 2H), 2.10 (m, 2H), 1.92 (m, 2H), 1.77 (m, 2H), 1.67 (m, 2H).

### Example 130P

### (8-fluoro-1,4-dioxaspiro[4.5]decan-8-yl)methyl acetate

To a mixture of Example 1300 (20 g), potassium iodide (1.312 g) and N,N-dimethylformamide (400 mL) was added potassium acetate (78 g) at 25° C. and the mixture was stirred at 135° C. for 16 hours. The mixture was poured into water (200 mL) and extracted with ethyl acetate (500 mL) three times, and the combined organic phase was washed with brine (250 mL) twice. The organic phase was dried over sodium sulfate, filtered, and concentrated. The crude material was purified by column chromatography (n-hexane/ethyl acetate=3:1) to provide the title compound. ¹H NMR (400 MHz, CDCl₃) δ ppm 4.22 (d, 2H), 3.88 (m, 4H), 2.21 (s, 3H), 1.95 (m, 2H), 1.88 (m, 2H), 1.73 (m, 2H), 1.71 (m, 2H)

### Example 130Q

### (8-fluoro-1,4-dioxaspiro[4.5]decan-8-yl)methanol

Example 130P (18 g) was dissolved into a mixed solution of tetrahydrofuran (200 mL) and water (100 mL) at 0° C. Lithium hydroxide monohydrate (6.51 g) was added, and the reaction mixture was stirred for 16 hours at 25° C. The mixture was extracted with ethyl acetate (500 mL) twice. The organic layers were combined, dried over sodium sulfate, filtered, and concentrated. The crude product was purified by column chromatography (n-hexane/ethyl acetate=3:1) to provide the title compound. ¹H NMR (400 MHz, CDCl₃) δ ppm 4.03-3.89 (m, 4H), 3.65-3.49 (m, 2H), 2.07-1.96 (m, 2H), 1.94-1.81 (m, 3H), 1.78-1.56 (m, 4H)

### Example 130R

### 2-(2-methoxyethoxy)ethyl 4-methylbenzenesulfonate

To a solution of sodium hydroxide (4.99 g) in water (100 mL) was added a solution of 2-(2-methoxyethoxyethanol (10 g) in tetrahydrofuran (100 mL) at 0° C. A solution of 4-methylbenzene-1-sulfonyl chloride (15.87 g) in tetrahydrofuran (100 mL) was added to the reaction at 0° C. The reaction was stirred at 25° C. for 10 hours. The reaction was diluted with 50 mL of water and extracted with ethyl acetate (3×50 mL). The combined organic layers were combined and concentrated to give a residue which was purified by column chromatography (eluting with petroleum:ethyl acetate=10:1 to 1:1) to give the desired product. ¹H NMR (400 MHz, CDCl₃) δ ppm 7.77 (d, 2H), 7.32 (d, 2H), 4.18-4.11 (m, 2H), 3.69-3.64 (m, 2H), 3.58-3.53 (m, 2H), 3.47-3.42 (m, 2H), 3.32 (s, 3H), 2.42 (s, 3H).

### Example 130S

### 4-fluoro-4-((2-(2-methoxyethoxy)ethoxy)methyl)cyclohexanone

To a solution of Example 130Q (5 g) in tetrahydrofuran (100 mL) was added NaH (2.103 g 60% in mineral oil) at 0° C. The reaction mixture was stirred at 25° C. for 15 minutes. Example 130R (9.37 g) was added to the reaction at 25° C. The reaction was stirred at 50° C. for 10 hours. After quenching with ice-cooled aqueous ammonium chloride solution (50 mL), the aqueous layer was extracted with ethyl acetate (3×50 mL). The combined organic layers were dried, filtered, and concentrated. The crude product was purified by column chromatography (eluting with petroleum ether:ethyl acetate=10:1 to 1:1) to give the pure ketal. The ketal was taken up in tetrahydrofuran (50 mL), and to it was added a solution of 6M aqueous hydrochloric acid (50 mL) at 0° C. The reaction was stirred at 25° C. for 10 hours. After adjustment of the solution with sodium hydroxide powder to pH 9, the aqueous layer was extracted with ethyl acetate (3×100 mL). The combined organic layers were dried, filtered, and concentrated. The crude material was purified by column chromatography (eluting with petroleum ether:ethyl acetate=10:1 to 1:1) to give the title compound, which was carried on without characterization.

### Example 130T

### 4-fluoro-4-((2-(2-methoxyethoxy)ethoxy)methyl)cyclohex-1-en-1-yl 1,1,2,2,3,3,4,4,4-nonafluorobutane-1-sulfonate

To a solution of Example 130S (2 g) and nonafluorobutanesulfonyl fluoride (10.95 g) in dry N,N-dimethylformamide (20 mL) was added (tert-butylimino)tris(pyrrolidino)phosphorene (11.33 g) dropwise at 0° C. Then the reaction mixture was stirred at 20° C. for 12 hours. The mixture was extracted with ethyl acetate (3×100 mL) and the combined extracts were washed with water (200 mL), dried over sodium sulfate, filtered and concentrated. The crude material was purified by column chromatography on silica gel and was eluted with petroleum ether/ethyl acetate=10/90 to 60/40 to afford the title compound. ¹H NMR (400 MHz, CDCl₃) δ ppm 5.73-5.67 (m, 1H), 3.74-3.70 (m, 2H), 3.68-3.63 (m, 4H), 3.62 (s, 1H), 3.58-3.54 (m, 3H), 3.41-3.37 (m, 3H), 2.62 (ddt, 1H), 2.51 (br s, 1H), 2.46 (br d, 1H), 2.35 (br dd, 1H), 2.182.09 (m, 1H), 2.01-1.83 (m, 1H).

### Example 130U

### 2-(4-fluoro-4-((2-(2-methoxyethoxy)ethoxy)methyl)cyclohex-1-en-1-yl)-4,4,5,5-tetramethyl-1,3,2-dioxaborolane

To a solution of Example 130T (2 g) in dimethoxyethane (20 mL) was added 4,4,4′,4′,5,5,5′,5′-octamethyl-2,2′-bi(1,3,2-dioxaborolane) (1.049 g).potassium acetate (1.106 g), PdCl₂ (dppf) (1,1′-bis(diphenylphosphino)ferrocenedichloro palladium (II) dichloromethane complex) (0.137 g) and 1,1′-bis(diphenylphosphino)ferrocene (0.104 g). The mixture was stirred at 80° C. for 12 hours under nitrogen. The reaction was filtered and the filtrate was concentrated under reduced pressure to give a crude product, which was purified by column chromatography on silica gel eluting with petroleum/ethyl acetate=10/90 to 30/70 to afford the title compound. ¹H NMR (400 MHz, CDCl₃) δ ppm 6.47-6.40 (m, 1H), 3.73-3.69 (m, 2H), 3.68-3.64 (m, 4H), 3.58-3.53 (m, 3H), 3.52 (s, 1H), 3.39 (s, 3H), 2.36-2.30 (m, 2H), 2.41-2.29 (m, 1H), 2.26-2.16 (m, 1H), 1.95-1.86 (m, 1H), 1.81-1.66 (m, 1H), 1.26 (s, 12H).

### Example 130V

### (2-(4-fluoro-4-((2-(2-methoxyethoxy)ethoxy)methyl)cyclohex-1-en-1-yl)pyrimidin-4-yl)methanol

To a solution of Example 130U (535 mg) and (2-chloropyrimidin-4-yl)methanol (200 mg) in 1,4-dioxane (2 mL) was added Pd(Ph₃P)₄ (71.9 mg) and saturated aqueous sodium bicarbonate (0.5 mL). The mixture was stirred under nitrogen at 110° C. for 16 hours. The reaction was cooled to 25° C. the mixture was filtered, the filtrate was extracted with ethyl acetate (3×100 mL), and the organic phases were combined and washed with brine (2×100 mL). The organic phase was dried over magnesium sulfate, filtered, and concentrated to a crude product which was purified by column chromatography on silica gel (eluting with petroleum ether:ethyl acetate=1:5 to 3:5) to afford the title compound. ¹H NMR (400 MHz, CDCl₃) δ ppm 8.64 (d, 1H), 7.23 (br s, 1H), 7.07 (d, 1H), 4.74 (s, 2H), 3.76-3.74 (m, 2H), 3.73-3.65 (m, 6H), 3.63 (s, 1H), 3.56 (dd, 2H), 3.39 (s, 3H), 2.80 (br s, 2H), 2.64-2.56 (m, 2H), 2.20-2.11 (m, 1H), 1.98-1.82 (m, 1H).

### Example 130W

### (R)-(2-(4-fluoro-4-((2-(2-methoxyethoxy)ethoxy)methyl)cyclohex-1-en-1-yl)pyrimidin-4-yl)methanol

Example 130V (1 g) was separated into enantiomers by SFC under the following conditions to give the title compound: Instrument: Thar analytical SFC, Column: Chiralpak AD-3, 3 μm, 0.46 cm id×5 cm L. Mobile phase: A for SFC CO₂ and B for methanol (0.05% IPAm), Gradient: B in A from 10% to 40% in 3 minutes, Flow rate: 4.0 mL/minute, Wavelength: 220 nm, System Back Pressure: 100 bar. ¹H NMR (400 MHz, CDCl₃) δ ppm 8.64 (d, 1H), 7.24 (br s, 1H), 7.07 (d, 1H), 4.74 (s, 2H), 3.77-3.74 (m, 2H), 3.73-3.65 (m, 6H), 3.63 (s, 1H), 3.57-3.55 (m, 2H), 3.39 (s, 3H), 2.80 (br s, 2H), 2.66-2.54 (m, 2H), 2.18-2.11 (m, 1H), 1.98-1.84 (m, 1H).

### Example 130X

### (R)-(2-(4-fluoro-4-((2-(2-methoxyethoxy)ethoxy)methyl)cyclohex-1-en-1-yl)pyrimidin-4-yl)methyl methanesulfonate

To a solution of Example 130W (55 mg) in dichloromethane (1 mL) at a temperature of 5° C. triethylamine (0.068 mL) and methanesulfonyl chloride (0.019 mL) were added. The mixture was allowed to reach ambient temperature and stirring was continued for 30 minutes. Dichloromethane (3 mL) and water (4 mL) were added, the layers separated via Chromabond® PTS cartridge, the aqueous layer was extracted with dichloromethane (2 mL), and the combined organic layers concentrated in vacuo to provide the title compound, which was used in the next reaction without further purification. MS (APCI) m/z 419.2.

### Example 130Y

### tert-butyl (7R,16R)-19,23-dichloro-1-cyclobutyl-10-({2-[(4R*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 130N (50 mg) and Example 130X (36.5 mg) in N,N-dimethylformamide (1 mL), cesium carbonate (54 mg) was added and the mixture stirred for 20 hours at ambient temperature. Ethyl acetate (10 mL) and water (20 mL) were added, and the aqueous layer was extracted with ethyl acetate. The combined organic layers were washed with brine, dried over magnesium sulfate, filtered, and concentrated in vacuo. Purification by chromatography on silica gel using an ISCO CombiFlash® Companion MPLC (4 g Chromabond® SiOH column, eluting with 0-10% dichloromethane/methanol) provided the title compound. MS (APCI) m/z 1091.5 (M+H)⁺.

### Example 130Z

### (7R,16R)-19,23-dichloro-1-cyclobutyl-10-({2-[(4R*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 89D by replacing Example 89C with Example 130Y. Purification by HPLC (XSelect CSH C18 20×150 mm 5 μm column, gradient 5-100% acetonitrile+0.1% formic acid in water+0.1% formic acid) was followed by dissolution in dichloromethane (10 mL) and treatment with saturated aqueous NaHCO₃. The aqueous layer was extracted with dichloromethane, dried over magnesium sulfate, filtered and concentrated to give the title compound. ¹H NMR (600 MHz, dimethylsulfoxide-d₆) δ ppm 12.91 (s, 1H), 8.74 (d, 1H), 8.66 (s, 1H), 7.39 (d, 1H), 7.14 (m, 1H), 6.85 (d, 1H), 6.75 (dd, 1H), 6.25 (s, 1H), 5.77 (m, 1H), 5.16 (d, 1H), 5.08 (d, 1H), 4.85 (m, 1H), 4.50 (m, 2H), 3.60 (m, 4H), 3.54 (m, 6H), 3.43 (m, 2H), 3.24 (s, 3H), 3.18 (m, 1H), 2.89 (dd, 1H), 2.55-2.45 (m, 10H), 2.74-2.69 (m, 3H), 2.18 (s, 3H), 2.14-2.07 (m, 2H), 2.07-1.99 (m, 3H), 1.98 (s, 3H), 1.89 (s, 3H), 1.89-1.78 (m, 1H), 1.78-1.70 (m, 2H). MS (APCI) m/z 1035.6 (M+H)⁺.

### Example 131

### (7R,16R)-19,23-dichloro-1-cyclobutyl-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 131A

### (2-((1r,4r)-4-(2-(2-(2-methoxyethoxy)ethoxy)ethoxy)cyclohexyl)pyrimidin-4-yl)methyl methanesulfonate

The compound was prepared as described in Example 130X by replacing Example 130W with Example 57G (30 mg) to give the title compound. MS (APCI) m/z 433.3 (M+H)⁺.

### Example 131B

### tert-butyl (7R,16R)-19,23-dichloro-1-cyclobutyl-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 130Y by replacing Example 130X with Example 131A. Purification by chromatography on silica gel using an ISCO CombiFlash® Companion MPLC (4 g Chromabond® SiOH column, eluting with 0-10% dichloromethane/methanol) provided the title compound. MS (APCI) m/z 1105.5 (M+H)⁺.

### Example 131C

### (7R,16R)-19,23-dichloro-1-cyclobutyl-10-({2-[(1r,4r)-4-{2-[2-(2-methoxyethoxy)ethoxy]ethoxy}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 89D by replacing Example 89C with Example 131B. Purification by HPLC (XBridge C8 19×150 mm 5 μm column, gradient 5-100% acetonitrile+0.2% ammonium hydroxide in water+0.2% ammonium hydroxide) provided a crude material, which was further purified by chromatography on silica gel using an ISCO CombiFlash® Companion MPLC (4 g Chromabond® SiOH column, eluting with 0-50% dichloromethane/methanol). The obtained material then was purified again by HPLC (XSelect CSH C18 19×150 mm 5 μm, gradient 5-100% acetonitrile+0.1% trifluoroacetic acid in water+0.1% trifluoroacetic acid) followed by dissolution in dichloromethane (10 mL) and treatment with saturated aqueous NaHCO₃. The aqueous layer was extracted with dichloromethane, dried over magnesium sulfate, filtered and concentrated to give the title compound. ¹H NMR (600 MHz, dimethylsulfoxide-d₆) δ ppm 12.88 (s, 1H), 8.70 (d, 1H), 8.66 (s, 1H), 7.40 (d, 1H), 6.85 (d, 1H), 6.75 (dd, 1H), 6.24 (s, 1H), 5.77 (s, 1H), 5.12 (d, 1H), 5.05 (d, 1H), 4.86 (m, 1H), 4.48 (m, 2H), 3.62-3.54 (m, 1H), 3.60-3.48 (m, 11H), 3.45-3.41 (m, 2H), 3.24 (s, 3H), 3.18 (m, 1H), 2.87 (dd, 1H), 2.77 (m, 1H), 2.71 (m, 2H), 2.55-2.45 (m, 5H), 2.19 (s, 3H), 2.12-1.93 (m, 11H), 1.88 (s, 3H), 1.84 (m, 1H), 1.74 (m, 1H), 1.65-1.59 (m, 2H), 1.61-1.55 (m, 2H), 1.32-1.22 (m, 3H). MS (APCI) m/z 1049.6 (M+H)⁺.

### Example 132

### (7R,16R)-19,23-dichloro-1-cyclobutyl-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,16,17-tetrahydro-15H-18,21-etheno-13,9-(metheno)-6,14-dioxa-2-thia-3,5-diazacyclononadeca[1,2,3-cd]indene-7-carboxylic Acid

### Example 132A

### (S)(2-(4-fluoro-4-((2-(2-methoxyethoxy)ethoxy)methyl)cyclohex-1-en-1-yl)pyrimidin-4-yl)methanol

The title compound was also isolated during the preparation of Example 130W. ¹H NMR (400 MHz, CDCl₃) δ ppm 8.64 (d, 1H), 7.23 (br s, 1H), 7.07 (d, 1H), 4.74 (s, 2H), 3.77-3.73 (m, 2H), 3.73-3.64 (m, 6H), 3.62 (s, 1H), 3.58-3.54 (m, 2H), 3.39 (s, 3H), 2.85-2.75 (m, 2H), 2.63 (br s, 1H), 2.61-2.51 (m, 1H), 2.19-2.11 (m, 1H), 1.99-1.83 (m, 1H).

### Example 132B

### tert-butyl (7R,16R)-19,23-dichloro-1-cyclobutyl-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,16,17-tetrahydro-15H-18,21-etheno-13,9-(metheno)-6,14-dioxa-2-thia-3,5-diazacyclononadeca[1,2,3-cd]indene-7-carboxylate

The title compound was prepared as described in Example 89C by replacing 89B with Example 132A. MS (APCI) m/z 1091.4 (M+H)⁺.

### Example 132C

### (7R,16R)-19,23-dichloro-1-cyclobutyl-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,16,17-tetrahydro-15H-18,21-etheno-13,9-(metheno)-6,14-dioxa-2-thia-3,5-diazacyclononadeca[1,2,3-cd]indene-7-carboxylic Acid

The title compound was prepared as described in Example 89D by replacing Example 89C with 132B. ¹H NMR (600 MHz, dimethylsulfoxide-d₆) δ ppm 12.92 (s, 1H), 8.73 (d, 1H), 8.66 (s, 1H), 7.39 (d, 1H), 7.14 (m, 1H), 6.84 (d, 1H), 6.75 (dd, 1H), 6.24 (m, 1H), 5.78 (d, 1H), 5.16 (d, 1H), 5.08 (d, 1H), 4.86 (m, 1H), 4.54-4.45 (m, 2H), 3.64-3.51 (m, 10H), 3.46-3.41 (m, 2H), 3.24 (s, 3H), 3.18 (m, 1H), 2.88 (dd, 1H), 2.71 (m, 3H), 2.55-2.45 (m, 7H), 2.19 (s, 3H), 2.10 (m, 2H), 2.03 (m, 3H), 1.99 (s, 3H), 1.88 (s, 3H), 1.85-1.74 (m, 4H). MS (APCI) m/z 1035.6 (M+H)⁺.

### Example 133

### (7R,16R)-19,23-dichloro-1-(4-fluorophenyl)-10-({2-[2-(2-{[(3R,3aR,6R,6aR)-6-methoxyhexahydrofuro[3,2-b]furan-3-yl]oxy}ethoxy)ethoxy]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 133A

### (3R,3aR,6R,6aR)-6-methoxyhexahydrofuro[3,2-b]furan-3-ol

To a suspension of (3R,3aR,6R,6aR)-hexahydrofuro[3,2-b]furan-3,6-diol (10.1 g) and silver oxide (24 g) at room temperature was added methyl iodide (6.5 mL), and the reaction was allowed to stir in the dark for 2 days. More methyl iodide (2.2 mL) and silver oxide (8 g) was added, and the reaction was stirred for 4 days. The reaction was filtered over diatomaceous earth, washing with dichloromethane, and concentrated. The residue was purified by normal phase MPLC on a Teledyne Isco CombiFlash® Rf+220 g gold silica gel column eluting with 5-85% ethyl acetate in heptanes to give the title compound. ¹H NMR (400 MHz, CDCl₃) δ ppm 4.59-4.53 (m, 1H), 4.51-4.47 (m, 1H), 4.33-4.21 (m, 1H), 4.11-4.02 (m, 1H), 4.01-3.89 (m, 2H), 3.74-3.61 (m, 2H), 3.46 (s, 3H), 2.85 (d, 1H).

### Example 133B

### (2-(2-bromoethoxy)ethoxy)(tert-butyl)dimethylsilane

To a solution of 2-(2-bromoethoxy)ethanol (2.4 g) and tert-butyldimethylchlorosilane (2.4 g) in N,N-dimethyl formamide (14.3 mL) was added N,N-diisopropylethylamine (6.2 mL), and the reaction was allowed to stir for 6 hours. The reaction was diluted with ethyl acetate, water and brine. The aqueous layer was extracted with ethyl acetate three times. The combined organic layers were washed with water then brine, dried over anhydrous sodium sulfate, filtered and concentrated. The residue was purified by normal phase MPLC on a Teledyne Isco CombiFlash® Rf+80 g gold silica gel column eluting with 0-15% ethyl acetate in heptanes to give the title compound. ¹H NMR (500 MHz, CDCl₃) δ ppm 3.84-3.80 (m, 2H), 3.79-3.75 (m, 2H), 3.60-3.56 (m, 2H), 3.48-3.43 (m, 2H), 0.90 (s, 9H), 0.07 (s, 6H).

### Example 133C

### tert-butyl (2-(2-(((3R,3aR,6R,6aR)-6-methoxyhexahydrofuro[3,2-b]furan-3-yl)oxy)ethoxy)ethoxy)dimethylsilane

To a solution of Example 133A (1.35 g) and Example 133B (3.58 g) in acetonitrile (50 mL) at room temperature was added sodium hydride (675 mg, 60% oil dispersion) slowly, and the reaction was heated to 50° C. for 24 hours. The reaction was cooled, diluted with saturated ammonium chloride and water and extracted with ethyl acetate three times. The combined organic layers were dried over anhydrous sodium sulfate, filtered and concentrated. The residue was purified by normal phase MPLC on a Teledyne Isco CombiFlash® Rf+80 g gold silica gel column eluting with 10-65% ethyl acetate in heptanes to give the title compound. ¹H NMR (400 MHz, dimethylsulfoxide-d₆) δ ppm 4.54-4.45 (m, 2H), 4.07-3.95 (m, 1H), 3.92-3.82 (m, 3H), 3.72-3.64 (m, 3H), 3.58-3.49 (m, 3H), 3.48-3.36 (m, 4H), 3.30 (s, 3H), 0.86 (s, 9H), 0.04 (s, 6H).

### Example 133D

### 2-(2-(((3R,3aR,6R,6aR)-6-methoxyhexahydrofuro[3,2-b]furan-3-yl)oxy)ethoxy)ethanol

To a solution of Example 133C (1.1 g) in tetrahydrofuran (10.4 mL) and methanol (5.2 mL) was added cesium fluoride (2.4 g), and the reaction was allowed to stir overnight. The reaction was concentrated, and the residue was taken up in ethyl acetate, filtered over diatomaceous earth and concentrated. The residue was purified by normal phase MPLC on a Teledyne Isco CombiFlash® Rf+40 g gold silica gel column eluting with 5-85% ethyl acetate in heptanes to give the title compound. ¹H NMR (400 MHz, dimethylsulfoxide-d₆) δ ppm 4.61-4.46 (m, 3H), 4.07-3.95 (m, 1H), 3.94-3.82 (m, 3H), 3.73-3.60 (m, 1H), 3.58-3.37 (m, 9H), 3.30 (s, 3H).

### Example 133E

### 4-(((tert-butyldimethylsilyl)oxy)methyl)-2-(2-(2-(((3R,3aR,6R,6aR)-6-methoxyhexahydrofuro[3,2-b]furan-3-yl)oxy)ethoxy)ethoxy)pyrimidine

To a solution of Example 133D (460 mg) and Example 38A (400 mg) in acetonitrile (5.2 mL) at 0° C. was added sodium hydride (185 mg, 60% oil dispersion), and the reaction was allowed to stir at room temperature for 5 hours. The reaction was diluted with saturated aqueous ammonium chloride and water and extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 40-90% ethyl acetate in heptanes to give the title compound. ¹H NMR (500 MHz, dimethylsulfoxide-d₆) δ ppm 8.59 (d, 1H), 7.13 (d, 1H), 4.66 (s, 2H), 4.54-4.46 (m, 2H), 4.43-4.34 (m, 2H), 4.06-3.95 (m, 1H), 3.91-3.81 (m, 3H), 3.78-3.71 (m, 2H), 3.70-3.62 (m, 1H), 3.61-3.52 (m, 3H), 3.45-3.36 (m, 2H), 3.29 (s, 3H), 0.92 (s, 9H), 0.10 (s, 6H).

### Example 133F

### (2-(2-(2-(((3R,3aR,6R,6aR)-6-methoxyhexahydrofuro[3,2-b]furan-3-yl)oxy)ethoxy)ethoxy)pyrimidin-4-yl)methanol

To a solution of Example 133E (180 mg) in tetrahydrofuran (1.3 mL) and methanol (650 μL) was added cesium fluoride (300 mg), and the reaction was allowed to stir for 24 hours. The reaction mixture was concentrated, and the residue was taken up in ethyl acetate, filtered over diatomaceous earth and concentrated. The residue was purified by normal phase MPLC on a Teledyne Isco CombiFlash® Rf+4 g gold silica gel column eluting with 0-7% methanol in dichloromethane to give the title compound. ¹H NMR (400 MHz, dimethylsulfoxide-d₆) δ ppm 8.55 (d, 1H), 7.18 (d, 1H), 5.63-5.53 (m, 1H), 4.56-4.43 (m, 4H), 4.42-4.34 (m, 2H), 4.08-3.95 (m, 1H), 3.93-3.80 (m, 3H), 3.79-3.63 (m, 3H), 3.61-3.50 (m, 3H), 3.46-3.36 (m, 2H), 3.29 (s, 3H).

### Example 133G

### tert-butyl (7R,16R)-19,23-dichloro-1-(4-fluorophenyl)-10-({2-[2-(2-{[(3R,3aR,6R,6aR)-6-methoxyhexahydrofuro[3,2-b]furan-3-yl]oxy}ethoxy)ethoxy]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A vial containing Example 133F (53 mg), Example 16N (40 mg), triphenylphosphine (39 mg) and N,N,N′,N′-tetramethylazodicarboxamide (26 mg) in toluene (120 μL) and tetrahydrofuran (120 μL) was allowed to stir at 50° C. overnight. The reaction was cooled, diluted with ethyl acetate, filtered over diatomaceous earth and concentrated. The residue was purified by normal phase MPLC on a Teledyne Isco CombiFlash® Rf+4 g gold silica gel column eluting with 0.5-10% methanol in dichloromethane to give the title compound.

### Example 133H

### (7R,16R)-19,23-dichloro-1-(4-fluorophenyl)-10-({2-[2-(2-{[(3R,3aR,6R,6aR)-6-methoxyhexahydrofuro[3,2-b]furan-3-yl]oxy}ethoxy)ethoxy]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 133G (54 mg) in dichloromethane (240 μL) was added trifluoroacetic acid (240 μL), and the reaction was allowed to stir overnight. The reaction was concentrated under a stream of nitrogen and taken up in water and acetonitrile. The mixture was purified by RP-HPLC on a Gilson PLC 2020 using a Luna™ column (250×50 mm, 10 mm) (30-80% over 30 minutes with acetonitrile in water containing 10 mM ammonium acetate) to give the title compound. ¹H NMR (400 MHz, dimethylsulfoxide-d₆) δ ppm 8.73 (s, 1H), 8.57 (d, 1H), 7.28-7.08 (m, 6H), 6.83 (d, 1H), 6.78-6.70 (m, 1H), 6.26-6.17 (m, 1H), 5.85-5.77 (m, 1H), 5.17-4.96 (m, 2H), 4.93-4.80 (m, 1H), 4.54-4.37 (m, 5H), 4.06-3.95 (m, 1H), 3.91-3.81 (m, 3H), 3.79-3.50 (m, 6H), 3.45-3.35 (m, 2H), 3.28 (s, 3H), 3.00-2.89 (m, 1H), 2.77-2.59 (m, 2H), 2.45 (br s, 4H), 2.23 (s, 3H), 2.02-1.92 (m, 6H). MS (ESI) m/z 1089.2 (M−H)⁻.

### Example 134

### (7R,16R)-19,23-dichloro-1-(4-fluorophenyl)-10-{[2-(2-{[(3R,3aR,6R,6aR)-6-methoxyhexahydrofuro[3,2-b]furan-3-yl]oxy}ethoxy)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 134A

### tert-butyl (2-(((3R,3aR,6R,6aR)-6-methoxyhexahydrofuro[3,2-b]furan-3-yl)oxy)ethoxy)dimethylsilane

To a solution of Example 133A (2 g) and (2-bromoethoxy) (tert-butyl)dimethylsilane (6 g) in acetonitrile (83 mL) at room temperature was added sodium hydride (1 g, 60% oil dispersion) slowly, and the reaction was heated to 50° C. for 24 hours. The reaction was cooled, diluted with saturated ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+120 g gold silica gel column eluting with 0-45% ethyl acetate in dichloromethane to give the title compound. ¹H NMR (500 MHz, CDCl₃) δ ppm 4.59-4.52 (m, 2H), 4.17-4.09 (m, 1H), 4.08-4.01 (m, 2H), 3.97-3.88 (m, 1H), 3.82-3.64 (m, 5H), 3.61-3.52 (m, 1H), 3.45 (s, 3H), 0.88 (s, 9H), 0.05 (s, 6H).

### Example 134B

### 2-(((3R,3aR,6R,6aR)-6-methoxyhexahydrofuro[3,2-b]furan-3-yl)oxy)ethanol

To a solution of Example 134A (2.1 g) in tetrahydrofuran (22 mL) and methanol (11 mL) was added cesium fluoride (5 g), and the reaction was allowed to stir overnight. The reaction was concentrated, and the residue was taken up in ethyl acetate, filtered over diatomaceous earth and concentrated. The residue was purified by normal phase MPLC on a Teledyne Isco CombiFlash® Rf+80 g gold silica gel column eluting with 0-6% methanol in dichloromethane to give the title compound. ¹H NMR (500 MHz, dimethylsulfoxide-d₆) δ ppm 4.60-4.47 (m, 3H), 4.05-3.97 (m, 1H), 3.93-3.83 (m, 3H), 3.62-3.53 (m, 1H), 3.52-3.37 (m, 5H), 3.30 (s, 3H).

### Example 134C

### 4-(((tert-butyldimethylsilyl)oxy)methyl)-2-(2-(((3R,3aR,6R,6aR)-6-methoxyhexahydrofuro[3,2-b]furan-3-yl)oxy)ethoxy)pyrimidine

To a solution of Example 134B (380 mg) and Example 38A (400 mg) in acetonitrile (5.2 mL) at 0° C. was added sodium hydride (185 mg, 60% oil dispersion), and the reaction was allowed to stir at room temperature for three hours. The reaction was cooled to 0° C., quenched with saturated ammonium chloride and water and extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 20-75% ethyl acetate in heptanes to give the title compound. ¹H NMR (400 MHz, dimethylsulfoxide-d₆) δ ppm 8.59 (d, 1H), 7.14 (d, 1H), 4.66 (s, 2H), 4.56-4.48 (m, 2H), 4.45-4.31 (m, 2H), 4.14-4.01 (m, 1H), 3.95-3.81 (m, 4H), 3.80-3.70 (m, 1H), 3.48-3.36 (m, 2H), 3.30 (s, 3H), 0.92 (s, 9H), 0.10 (s, 6H).

### Example 134D

### (2-(2-(((3R,3aR,6R,6aR)-6-methoxyhexahydrofuro[3,2-b]furan-3-yl)oxy)ethoxy)pyrimidin-4-yl)methanol

To a solution of Example 134C (260 mg) in tetrahydrofuran (2 mL) and methanol (1 mL) was added cesium fluoride (460 mg), and the reaction was allowed to stir overnight. The reaction was concentrated, and the residue was taken up in ethyl acetate, filtered over diatomaceous earth and concentrated. The residue was purified by normal phase MPLC on a Teledyne Isco CombiFlash® Rf+12 g gold silica gel column eluting with 0-7% methanol in dichloromethane to give the title compound. ¹H NMR (400 MHz, dimethylsulfoxide-d₆) δ ppm 8.56 (d, 1H), 7.18 (d, 1H), 5.64-5.53 (m, 1H), 4.60-4.30 (m, 6H), 4.15-4.01 (m, 1H), 3.96-3.82 (m, 4H), 3.80-3.69 (m, 1H), 3.50-3.37 (m, 2H), 3.30 (s, 3H).

### Example 134E

### tert-butyl (7R,16R)-19,23-dichloro-1-(4-fluorophenyl)-10-{[2-(2-{[(3R,3aR,6R,6aR)-6-methoxyhexahydrofuro[3,2-b]furan-3-yl]oxy}ethoxy)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A vial containing Example 134D (46 mg), Example 16N (40 mg), triphenylphosphine (39 mg) and N,N,N′,N′-tetramethylazodicarboxamide (26 mg) in toluene (120 μL) and tetrahydrofuran (120 μL) was allowed to stir at 50° C. overnight. The reaction was cooled, diluted with ethyl acetate, filtered over diatomaceous earth and the filtrate was concentrated. The residue was purified by normal phase MPLC on a Teledyne Isco CombiFlash® Rf+4 g gold silica gel column eluting with 1-10% methanol in dichloromethane to give the title compound. ¹H NMR (400 MHz, dimethylsulfoxide-d₆) δ ppm 8.74 (s, 1H), 8.61 (d, 1H), 7.29-7.13 (m, 5H), 6.89 (d, 1H), 6.83 (dd, 1H), 6.04 (dd, 1H), 5.67 (d, 1H), 5.16-4.96 (m, 2H), 4.81-4.69 (m, 1H), 4.58-4.34 (m, 6H), 4.14-4.03 (m, 1H), 3.96-3.73 (m, 5H), 3.65 (dd, 1H), 3.48-3.39 (m, 2H), 3.30 (s, 3H), 2.93-2.75 (m, 3H), 2.72-2.59 (m, 2H), 2.39 (br s, 2H), 2.30 (br s, 2H), 2.14 (s, 3H), 2.10 (s, 3H), 1.90 (s, 3H), 1.06 (s, 9H).

### Example 134F

### (7R,16R)-19,23-dichloro-1-(4-fluorophenyl)-10-{[2-(2-{[(3R,3aR,6R,6aR)-6-methoxyhexahydrofuro[3,2-b]furan-3-yl]oxy}ethoxy)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 134E (54 mg) in dichloromethane (240 μL) was added trifluoroacetic acid (240 μL), and the reaction was allowed to stir overnight. The reaction was concentrated under a stream of nitrogen and was taken up in water and acetonitrile. The mixture was purified by RP-HPLC on a Gilson PLC 2020 using a Luna™ column (250×50 mm, 10 mm) (30-80% over 30 minutes with acetonitrile in water containing 10 mM ammonium acetate) to give the title compound. ¹H NMR (500 MHz, dimethylsulfoxide-d₆) δ ppm 8.73 (s, 1H), 8.57 (d, 1H), 7.27-7.08 (m, 6H), 6.82 (d, 1H), 6.77-6.69 (m, 1H), 6.25-6.16 (m, 1H), 5.87-5.78 (m, 1H), 5.14-4.97 (m, 2H), 4.93-4.81 (m, 1H), 4.58-4.49 (m, 2H), 4.48-4.35 (m, 4H), 4.13-4.03 (m, 1H), 3.94-3.83 (m, 4H), 3.81-3.71 (m, 1H), 3.65-3.55 (m, 1H), 3.47-3.38 (m, 2H), 3.30 (s, 3H), 2.99-2.88 (m, 1H), 2.76-2.59 (m, 2H), 2.44 (br s, 4H), 2.22 (s, 3H), 2.02-1.92 (m, 6H). MS (ESI) m/z 1045.0 (M−H)⁻.

### Example 135

### (7R,16R)-19,23-dichloro-1-(cyclopent-1-en-1-yl)-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 135A

### tert-butyl (7R,16R)-19,23-dichloro-1-(cyclopent-1-en-1-yl)-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 101L by replacing Example 101J and Example 16N with Example 86F and Example 125Q, respectively. MS (ESI) m/z 552.0 (M+H)²⁺.

### Example 135B

### (7R,16R)-19,23-dichloro-1-(cyclopent-1-en-1-yl)-10-({2-[(4S*)-4-fluoro-4-{[2-(2-methoxyethoxy)ethoxy]methyl}cyclohex-1-en-1-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 101M by replacing Example 101L with Example 135A. ¹H NMR (500 MHz, dimethylsulfoxide-d₆) δ ppm 9.53 (s, 1H), 8.69 (d, 1H), 8.62 (s, 1H), 7.33 (d, 1H), 7.08 (dt, 1H), 6.83-6.74 (m, 2H), 6.20 (dd, 1H), 5.74 (td, 1H), 5.69 (d, 1H), 5.10 (d, 1H), 5.03 (d, 1H), 4.90-4.84 (m, 1H), 4.50-4.39 (m, 2H), 3.50-3.44 (m, 13H), 3.40-3.34 (m, 3H), 3.17 (s, 3H), 2.88 (d, 1H), 2.75 (s, 3H), 2.66 (d, 1H), 2.47 (dd, 2H), 2.27 (tq, 2H), 1.96 (d, 1H), 1.94 (s, 3H), 1.89 (s, 5H), 1.74-1.63 (m, 3H). MS (ESI) m/z 1049.5 (M+H)⁺.

### Example 136

### (7R,16R)-19,23-dichloro-10-[(2-{4-[(2S)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 136A

### (S)-2-(4-(2,3-dimethoxypropoxy)phenyl)-4,4,5,5-tetramethyl-1,3,2-dioxaborolane

(R)-2,3-Dimethoxypropan-1-ol (109 mg), 4-hydroxyphenylboronic acid pinacol ester (200 mg). N,N,N′,N′-tetramethylazodicarboxamide (626 mg) and triphenylphosphine (953 mg) were combined and flushed with argon for 15 minutes. Tetrahydrofuran (1.0 mL) and toluene (1.0 mL) were flushed with argon for 15 minutes and then combined with the reactants. The mixture was stirred over the weekend at room temperature. The reaction mixture was concentrated. Purification was performed on a silica gel column (12 g, 0-30% methanol in dichloromethane and 12 g, 0-40% acetone in n-heptane). The desired fractions were combined and the solvents were removed under reduced pressure to provide the title compound. MS (ESI) m/z 323.2 (M+H)⁺.

### Example 136B

### (S)-(2-(4-(2,3-dimethoxypropoxy)phenyl)pyrimidin-4-yl)methanol

Example 136A (235 mg), (2-chloropyrimidin-4-yl)methanol (74 mg), and tetrakis(triphenylphosphine)palladium (30 mg) were dissolved in tetrahydrofuran (6.0 mL). Aqueous sodium bicarbonate solution (6 mL, 9%) was added under argon atmosphere. The reaction was heated for 4 hours at 120° C. in the microwave. The reaction mixture was diluted with ethyl acetate and water. The aqueous layer was washed with ethyl acetate (three times). The combined organic layers were dried over magnesium sulfate, filtered, and concentrated. Purification was performed on a silica gel column (12 g, 0-40% acetone in n-heptane). The desired fractions were combined and the solvents were removed under reduced pressure to provide the title compound. MS (ESI) m/z 305.2 (M+H)⁺.

### Example 136C

### tert-butyl (7R,16R)-19,23-dichloro-10-[(2-{4-[(2S)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 136B (25 mg), Example 16N (20 mg), triphenylphosphine (26 mg), and N,N,N′,N′-tetramethylazodicarboxamide (17 mg) were combined and flushed with argon for 15 minutes. Tetrahydrofuran (0.2 mL) and toluene (1.0 mL) were mixed, flushed with argon for 15 minutes, and added to the reactants. The reaction mixture was stirred at room temperature overnight. The reaction mixture was concentrated. Purification was performed on a silica gel column (4 g, 0-30% methanol in dichloromethane). The desired fractions were combined and the solvents were removed under reduced pressure to provide the title compound. MS (APCI) m/z 1195.6 (M+H)⁺.

### Example 136D

### (7R,16R)-19,23-dichloro-10-[(2-{4-[(2S)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 136C (43 mg) was dissolved in dichloromethane (238 μL) and trifluoroacetic acid (183 μL) was added. The reaction mixture was stirred at room temperature over the weekend. The reaction mixture was diluted with dichloromethane and aqueous sodium bicarbonate solution (9%). The aqueous layer was washed with dichloromethane (five times) and dried over sodium sulfate. Filtration, concentration, and purification by HPLC (Waters X-Bridge C8 19×150 mm 5 μm column, gradient 5-100% acetonitrile+0.2% NH₄OH in water+0.2% ammonium hydroxide) provided the title compound. ¹H NMR (600 MHz, dimethylsulfoxide-d₆) δ ppm 8.82 (d, 1H), 8.74 (s, 1H), 8.36-8.33 (m, 2H), 7.44 (d, 1H), 7.22-7.19 (m, 2H), 7.15-7.13 (m, 2H), 7.09-7.07 (m, 2H), 6.88 (d, 1H), 6.75 (dd, 1H), 6.23 (m, 1H), 5.81 (m, 1H), 5.25 (d, 1H), 5.17 (d, 1H), 4.86 (m, 1H), 4.46-4.42 (m, 2H), 4.16-4.13 (dd, 1H), 4.07-4.05 (dd, 1H), 3.71-3.68 (m, 1H), 3.65 (dd, 1H), 3.52 (qd, 2H), 3.39 (s, 6H), 2.99-2.97 (m, 1H), 2.67 (qd, 2H), 2.54-2.26 (m, 8H), 2.15 (s, 3H), 1.99 (s, 3H), 1.95 (s, 3H). MS (APCI) m/z 1039.3 (M+H)⁺.

### Example 137

### (7R,16R)-19,23-dichloro-10-[(2-{4-[(2R)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 137A

### (R)-2-(4-(2,3-dimethoxypropoxy)phenyl)-4,4,5,5-tetramethyl-1,3,2-dioxaborolane

The title compound was prepared by substituting (S)-2,3-dimethoxypropan-1-ol for (R)-2,3-dimethoxypropan-1-ol in Example 136A. MS (ESI) m/z 323.2 (M+H)⁺.

### Example 137B

### (R)-(2-(4-(2,3-dimethoxypropoxy)phenyl)pyrimidin-4-yl)methanol

The title compound was prepared by substituting Example 137A for Example 136A in Example 136B. MS (ESI) m/z 305.2 (M+H)⁺.

### Example 137C

### tert-butyl (7R,16R)-19,23-dichloro-10-[(2-{4-[(2R)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared by substituting Example 137B for Example 136B in Example 136C. MS (APCI) m/z 1195.6 (M+H)⁺.

### Example 137D

### (7R,16R)-19,23-dichloro-10-[(2-{4-[(2R)-2,3-dimethoxypropoxy]phenyl}pyrimidin-4-yl)methoxy]-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was prepared by substituting Example 137C for Example 136C in Example 136D. Purification by HPLC (Waters X-Bridge C8 19×150 mm 5 μm column, gradient 5-100% acetonitrile+0.2% NH₄OH in water+0.2% ammonium hydroxide) followed by a second purification by HPLC (Waters X-Bridge C8 19×150 mm 5 μm column, gradient 5-100% acetonitrile+0.1% trifluoroacetic acid in water+0.1% TFA) provided the title compound. ¹H NMR (600 MHz, dimethylsulfoxide-d₆) δ ppm 8.82 (d, 1H), 8.74 (s, 1H), 8.35-8.34 (m, 2H), 7.44 (d, 1H), 7.22-7.19 (m, 2H), 7.15-7.13 (m, 2H), 7.08-7.07 (m, 2H), 6.88 (d, 1H), 6.75 (dd, 1H), 6.23 (m, 1H), 5.80 (m, 1H), 5.25 (d, 1H), 5.18 (d, 1H), 4.87-4.84 (m, 1H), 4.46-4.42 (m, 2H), 4.14 (dd, 1H), 4.06 (dd, 1H), 3.71-3.68 (m, 1H), 3.65 (dd, 1H), 3.52 (qd, 2H), 3.39 (s, 6H), 2.99-2.97 (m, 1H), 2.67 (qd, 2H), 2.55-2.34 (m, 8H), 2.15 (s, 3H), 1.99 (s, 3H), 1.95 (s, 3H). MS (APCI) m/z 1039.4 (M+H)⁺.

### Example 138

### (7R,16R)-19,23-dichloro-1-(4-fluorophenyl)-10-[(2-{(1r,4r)-4-[2-(2-{[(3R,3aR,6R,6aR)-6-methoxyhexahydrofuro[3,2-b]furan-3-yl]oxy}ethoxy)ethoxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 138A

### (3R,3aR,6R,6aR)-3-(2-bromoethoxy)-6-methoxyhexahydrofuro[3,2-b]furan

To a solution of Example 134B (500 mg) in tetrahydrofuran (6.1 mL) in a water bath was added triphenylphosphine (770 mg) followed by carbon tetrabromide (970 mg), and the reaction was allowed to stir at room temperature for 2 hours. The reaction was filtered over diatomaceous earth and concentrated. The residue was purified by normal phase MPLC on a Teledyne Isco CombiFlash® Rf+40 g gold silica gel column eluting with 0-65% ethyl acetate in heptanes to give the title compound. ¹H NMR (500 MHz, dimethylsulfoxide-d₆) δ ppm 4.56-4.46 (m, 2H), 4.13-4.01 (m, 1H), 3.95-3.82 (m, 4H), 3.79-3.67 (m, 1H), 3.65-3.52 (m, 2H), 3.50-3.38 (m, 2H), 3.30 (s, 3H).

### Example 138B

### 2-((1r,4r)-4-(allyloxy)cyclohexyl)-4-(((tert-butyldiphenylsilyl)oxy)methyl)pyrimidine

To a suspension of sodium hydride (660 mg, 60% oil dispersion) in tetrahydrofuran (20 mL) at room temperature was added a solution of Example 57E (600 mg) in tetrahydrofuran (5 mL) dropwise, and the resulting suspension was stirred for 1 hour under nitrogen. To the mixture, allylbromide (400 mg) was added. The mixture was stirred for 4 hours at room temperature. The mixture was quenched with saturated aqueous ammonium chloride and extracted with ethyl acetate. The organic layer was washed with water and brine, dried over anhydrous sodium sulfate, filtered and concentrated. The residue was purified by silica gel chromatography on a 40 g column eluting with 20% ethyl acetate in heptanes to give the title compound. MS (ESI) m/z 487.0 (M+H)⁺.

### Example 138C

### 2-(((1r,4r)-4-(4-(((tert-butyldiphenylsilyl)oxy)methyl)pyrimidin-2-yl)cyclohexyl)oxy)acetaldehyde

A solution of Example 138B (530 mg) in tetrahydrofuran (13.6 mL) and water (13.6 mL) was treated with osmium tetroxide (350 μL, 4% by weight solution) and sodium periodate (930 mg), and the reaction was allowed to stir for 2 hours. The reaction was diluted with water and ethyl acetate. The aqueous layer was extracted with ethyl acetate three times, and the combined organic layers were dried over anhydrous sodium sulfate, filtered and concentrated to give the title compound that was used in the next step without further purification.

### Example 138D

### 2-(((1r,4r)-4-(4-(((tert-butyldiphenylsilyl)oxy)methyl)pyrimidin-2-yl)cyclohexyl)oxy)ethanol

To a solution of Example 138C (525 mg) in methanol (5.4 mL) at 0° C. was added sodium borohydride (41 mg), and the reaction was allowed to stir for 3 hours at room temperature and overnight at 4° C. Additional sodium borohydride (10 mg) was added at 0° C., and the reaction was allowed to warm to room temperature. After 1 hour, the reaction was cooled, quenched with saturated ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 0-6% methanol in dichloromethane to give the title compound. ¹H NMR (400 MHz, dimethylsulfoxide-d₆) δ ppm 8.75 (d, 1H), 7.68-7.60 (m, 4H), 7.53-7.37 (m, 7H), 4.73 (s, 2H), 4.54-4.48 (m, 1H), 3.52-3.39 (m, 4H), 3.29-3.19 (m, 1H), 2.75-2.63 (m, 1H), 2.10-1.98 (m, 2H), 1.96-1.84 (m, 2H), 1.62-1.45 (m, 2H), 1.32-1.15 (m, 2H), 1.06 (s, 9H).

### Example 138E

### 4-(((tert-butyldiphenylsilyl)oxy)methyl)-2-((1R,4r)-4-(2-(2-(((3R,3aR,6R,6aR)-6-methoxyhexahydrofuro[3,2-b]furan-3-yl)oxy)ethoxy)ethoxy)cyclohexyl)pyrimidine

To a solution of Example 138D (150 mg) and Example 138A (110 mg) in acetonitrile (1.5 mL) was added sodium hydride (24 mg, 60% oil dispersion), and the reaction was allowed to stir at 50° C. overnight. The reaction was cooled, quenched with saturated aqueous ammonium chloride and extracted with ethyl acetate three times. The combined organic layers were dried over anhydrous sodium sulfate, filtered and concentrated. The residue was purified by normal phase MPLC on a Teledyne Isco CombiFlash® Rf+24 g gold silica gel column eluting with 0-4% methanol in dichloromethane to give the title compound. ¹H NMR (400 MHz, dimethylsulfoxide-d₆) δ ppm 8.76 (d, 1H), 7.69-7.60 (m, 4H), 7.53-7.38 (m, 7H), 4.73 (s, 2H), 4.54-4.45 (m, 2H), 4.07-3.96 (m, 1H), 3.93-3.81 (m, 3H), 3.72-3.60 (m, 1H), 3.58-3.36 (m, 8H), 3.30-3.20 (m, 4H), 2.76-2.63 (m, 1H), 2.10-1.98 (m, 2H), 1.96-1.85 (m, 2H), 1.63-1.46 (m, 2H), 1.32-1.16 (m, 2H), 1.06 (s, 9H).

### Example 138F

### (2-((1R,4r)-4-(2-(2-(((3R,3aR,6R,6aR)-6-methoxyhexahydrofuro[3,2-b]furan-3-yl)oxy)ethoxy)ethoxy)cyclohexyl)pyrimidin-4-yl)methanol

To a solution of Example 138E (29 mg) in tetrahydrofuran (140 μL) and methanol (70 μL) was added cesium fluoride (33 mg), and the reaction was allowed to stir overnight. The reaction was concentrated, and the residue was taken up in ethyl acetate, filtered over diatomaceous earth and concentrated. The residue was purified by normal phase MPLC on a Teledyne Isco CombiFlash® Rf+4 g gold silica gel column eluting with 2-10% methanol in dichloromethane to give the title compound. ¹H NMR (400 MHz, dimethylsulfoxide-d₆) δ ppm 8.68 (d, 1H), 7.35 (d, 1H), 5.62-5.50 (m, 1H), 4.58-4.45 (m, 3H), 4.08-397 (m, 1H), 3.94-3.82 (m, 3H), 3.72-3.61 (m, 1H), 3.60-3.48 (m, 6H), 3.46-3.37 (m, 2H), 3.32-3.25 (m, 4H), 2.79-2.66 (m, 1H), 2.12-2.01 (m, 2H), 1.99-1.88 (m, 2H), 1.67-1.50 (m, 2H), 1.34-1.19 (m, 2H).

### Example 138G

### tert-butyl (7R,16R)-19,23-dichloro-1-(4-fluorophenyl)-10-[(2-{(1r,4r)-4-[2-(2-{[(3R,3aR,6R,6aR)-6-methoxyhexahydrofuro[3,2-b]furan-3-yl]oxy}ethoxy)ethoxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 138F (11.9 mg) and Example 16N (11 mg) were azeotroped with toluene and tetrahydrofuran three times. The residue was taken up in toluene (70 μL) and tetrahydrofuran (70 μL), and triphenylphosphine (7 mg) and N,N,N′,N′-tetramethylazodicarboxamide (4.7 mg) were added. The reaction was heated to 50° C. for 4 hours. The reaction was cooled, diluted with ethyl acetate, filtered over diatomaceous earth and the filtrate was concentrated. The residue was purified by normal phase MPLC on a Teledyne Isco CombiFlash® Rf+4 g gold silica gel column eluting with 1.5-10% methanol in dichloromethane to give the title compound.

### Example 138H

### (7R,16R)-19,23-dichloro-1-(4-fluorophenyl)-10-[(2-{(1r,4r)-4-[2-(2-{[(3R,3aR,6R,6aR)-6-methoxyhexahydrofuro[3,2-b]furan-3-yl]oxy}ethoxy)ethoxy]cyclohexyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 138G (14 mg) in dichloromethane (70 μL) was added trifluoroacetic acid (70 μL), and the reaction was allowed to stir overnight. The reaction was concentrated under a stream of nitrogen and taken up in water and acetonitrile. The mixture was purified by RP-HPLC on a Gilson PLC 2020 using a Luna™ column (250×50 mm, 10 mm) (30-80% over 30 minutes with acetonitrile in water containing 10 mM ammonium acetate) to give the title compound. ¹H NMR (500 MHz, dimethylsulfoxide-d₆) δ ppm 8.73 (s, 1H), 8.70 (d, 1H), 7.42 (d, 1H), 7.24-7.09 (m, 5H), 6.84 (d, 1H), 6.79-6.71 (m, 1H), 6.27-6.18 (m, 1H), 5.84-5.76 (m, 1H), 5.20-5.00 (m, 2H), 4.92-4.80 (m, 1H). 4.57-4.36 (m, 4H), 4.07-3.97 (m, 1H), 3.94-3.82 (m, 3H), 3.71-3.48 (m, 6H), 3.45-3.38 (m, 2H), 3.33-3.25 (m, 4H), 2.99-2.90 (m, 1H), 2.83-2.61 (m, 4H), 2.47 (br s, 4H), 2.51 (s, 3H), 2.12-2.02 (m, 2H), 2.01-1.91 (m, 6H), 1.67-1.53 (m, 2H), 1.35-1.20 (m, 2H). MS (ESI) m/z 1171.2 (M−H)⁻.

### Example 139

### (7R,16R)-19,23-dichloro-1-(4-fluorophenyl)-10-{[2-(3-{[2-(2-methoxyethoxy)ethoxy]methyl}azetidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 139A

### 4-(((tert-butyldimethylsilyl)oxy)methyl)-2-(3-((2-(2-methoxyethoxy)ethoxy)methyl)azetidin-1-yl)pyrimidine

Example 94A (250 mg) was dissolved in tetrahydrofuran (4.5 mL) and cooled to 0° C. with an ice-bath. Sodium hydride (465 mg, 50%) was added and the mixture was stirred at 0° C. for 1 hour. Tetrabutylammonium iodide (15 mg) and 1-bromo-2-(2-methoxyethoxy) ethane (493 mg, 90%) were added. The ice-bath was removed and the reaction mixture was stirred at room temperature for 1 hour. Methanol was added to the reaction mixture and the reaction mixture was concentrated. Purification was performed on a silica gel column (12 g, 0-50% methanol in dichloromethane). The desired fractions were combined and the solvents were removed under reduced pressure to provide the title compound. MS (ESI) m/z 412.3 (M+H)⁺.

### Example 139B

### (2-(3-((2-(2-methoxyethoxy)ethoxy)methyl)azetidin-1-yl)pyrimidin-4-yl)methanol

Example 139A (281 mg) was dissolved in tetrahydrofuran (1.0 mL) and cooled to 0° C. with an ice-bath. Tetrabutylammonium fluoride (1.37 mL, 1M) was added and the reaction mixture was stirred at 0° C. for 2 hours. The reaction mixture was concentrated. Purification was performed on a silica gel column (4 g, 0-20% methanol in dichloromethane). The desired fractions were combined and the solvents were removed under reduced pressure to provide the title compound. MS (ESI) m/z 298.2 (M+H)⁺.

### Example 139C

### (2-(3-((2-(2-methoxyethoxy)ethoxy)methyl)azetidin-1-yl)pyrimidin-4-yl)methyl methanesulfonate

Example 139B (30 mg) and triethylamine (0.04 mL) were dissolved in dichloromethane (1.0 mL). The mixture was cooled to 0° C. by an ice-bath. Methanesulfonyl chloride (9.29 μL) was added and the reaction mixture was stirred for 30 minutes while warming up to ambient temperature. To the reaction mixture was added brine. The aqueous layer was washed with dichloromethane. The organic layer was dried by a PTS cartridge, concentrated, and used in the next step without further purification. MS (ESI) m/z 376.2 (M+H)⁺.

### Example 139D

### tert-butyl (7R,16R)-19,23-dichloro-1-(4-fluorophenyl)-10-{[2-(3-{[2-(2-methoxyethoxy)ethoxy]methyl}azetidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 139C (35 mg), Example 16N 2 (30 mg), and cesium carbonate (36 mg) were dissolved in dimethyl formamide (200 μL) under argon atmosphere. The reaction mixture was stirred overnight at room temperature. To the reaction mixture aqueous sodium bicarbonate solution (5%) was added dropwise. Dichloromethane was added and the phases were separated. The aqueous layer was extracted with dichloromethane (twice). The organic layer was dried by a PTS-cartridge and concentrated. Purification was performed on a silica gel column (4 g, 0-38% methanol in dichloromethane). The desired fractions were combined and the solvents were removed under reduced pressure to provide the title compound. MS (APCI) m/z 1088.4 (M+H)⁺.

### Example 139E

### (7R,16R)-19,23-dichloro-1-(4-fluorophenyl)-10-{[2-(3-{[2-(2-methoxyethoxy)ethoxy]methyl}azetidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 139D (73 mg) was dissolved in dichloromethane (1.0 mL) and trifluoroacetic acid (260 μL) was added. The reaction mixture was stirred overnight at room temperature. Aqueous sodium bicarbonate solution (9%) and dichloromethane were added dropwise to the reaction mixture. The aqueous layer was extracted with dichloromethane (five times). The organic layer was dried over sodium sulfate, filtered, and concentrated. The crude material was purified by HPLC (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.69 (s, 1H), 8.29 (d, 1H), 7.21-7.18 (m, 2H), 7.14-7.11 (m, 2H), 6.79-6.76 (m, 2H), 6.70-6.68 (m, 1H), 6.10 (m, 1H), 5.88 (m, 1H), 4.96-4.86 (m, 3H), 4.47-4.39 (m, 2H), 4.09-4.05 (m, 2H), 3.75 (dd, 2H), 3.60 (d, 2H), 3.56-3.49 (m, 7H), 3.42-3.40 (m, 2H), 3.22 (s, 3H), 2.93-2.85 (m, 2H), 2.72-2.66 (m, 2H), 2.55-2.30 (m, 8H), 2.17 (s, 3H), 2.00 (s, 3H), 1.92 (s, 3H). MS (APCI) m/z 1032.3 (M+H)⁺.

### Example 140

### (7S,16R)-19,23-dichloro-1-(4-fluorophenyl)-10-{[2-(3-{[2-(2-methoxyethoxy)ethoxy]methyl}azetidin-1-yl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

The title compound was obtained as a minor product from the preparation for Example 139E after purification by HPLC (Waters X-Bridge C8 19×150 mm 5 μm column, gradient 5-100% acetonitrile+0.2% ammonium hydroxide in water+0.2% ammonium hydroxide). ¹H NMR (600 MHz, dimethylsulfoxide-d₆) δ ppm 8.67 (s, 1H), 8.30 (d, 1H), 7.20-7.17 (m, 2H), 7.13-7.11 (m, 2H), 6.87-6.86 (m, 1H), 6.81 (d, 1H), 6.68-6.66 (m, 1H), 6.16 (m, 1H), 5.98 (m, 1H), 5.13 (m, 1H), 4.93 (d, 1H), 4.89 (d, 1H), 4.22 (t, 1H), 4.11 (d, 1H), 4.07 (t, 2H), 3.75 (dd, 2H), 3.60 (d, 2H), 3.56-3.50 (m, 7H), 3.42-3.40 (m, 3H), 3.22 (s, 3H), 3.13-3.09 (m, 1H), 2.92-2.85 (m, 1H), 2.75-2.72 (m, 1H), 2.52-2.45 (m, 8H), 2.26 (s, 3H), 2.19 (s, 3H), 1.72 (s, 3H). MS (APCI) m/z 1032.3 (M+H)⁺.

### Example 141

### (7R,16R)-19,23-dichloro-10-({2-[(1,3-dimethoxypropan-2-yl)oxy]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 141A

### 4-(((tert-butyldimethylsilyl)oxy)methyl)-2-((1,3-dimethoxypropan-2-yl)oxy)pyrimidine

1,3-Dimethoxypropan-2-ol (279 mg), Example 38A (200 mg.), palladium acetate (17 mg), ((RS)2,2′-bis(diphenylphosphino)-1,1′-binaphthyl) (96 mg), and cesium carbonate (755 mg) were suspended in toluene (3 mL) under an argon atmosphere. The reaction mixture was heated 125° C. for 1 hour in t a Biotage® Initiator microwave. The reaction mixture was concentrated and the residue was absorbed on Bulk Isolute Sorbent. Purification was performed on a silica gel column (12 g, 0-30% methanol in dichloromethane). The desired fractions were combined and the solvents were removed under reduced pressure to provide the title compound. MS (APCI) m/z 343.2 (M+H)⁺.

### Example 141B

### (2-((1,3-dimethoxypropan-2-yl)oxy)pyrimidin-4-yl)methanol

Example 141A (214 mg) was dissolved in tetrahydrofuran (1.0 mL) and cooled to 0° C. by an ice bath. Tetrabutylammonium fluoride (1M solution in tetrahydrofuran, 1.25 mL) was added and the reaction mixture was stirred at 0° C. for 2 hours. The reaction mixture was concentrated and the residue was absorbed on Bulk Isolute Sorbent. Purification was performed on a silica gel column (4 g, 0-20% methanol in dichloromethane). The desired fractions were combined and the solvents were removed under reduced pressure to provide the title compound. MS (APCI) m/z 229.2 (M+H)⁺.

### Example 141C

### (2-((1,3-dimethoxypropan-2-yl)oxy)pyrimidin-4-yl)methyl methanesulfonate

Example 141B (23 mg) and triethylamine (42 μL) were dissolved in dichloromethane (1.0 mL) and cooled to 0° C. by an ice-bath. Methanesulfonyl chloride (9.36 μL) was added and the reaction mixture was stirred for 15 minutes while warming up to room temperature. Brine was added to the reaction mixture and the phases were separated. The aqueous layer was washed with dichloromethane. The organic layer was dried by PTS cartridge and concentrated to yield the crude title product. MS (APCI) m/z 307.2 (M+H)⁺.

### Example 141D

### tert-butyl (7R,16R)-19,23-dichloro-10-({2-[(1,3-dimethoxypropan-2-yl)oxy]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 141C (28 mg), Example 16N (25 mg), and cesium carbonate (36 mg) were suspended in N,N-dimethyl formamide (0.5 mL) under argon atmosphere. The reaction mixture was stirred overnight at room temperature. An aliquot analyzed by LC/MS indicated full conversion. The reaction mixture was diluted with dichloromethane and washed with brine. The aqueous layer was extracted with dichloromethane (four times). The organic layer was dried by a PTS-Cartridge and concentrated. The residue was absorbed on Bulk Isolute Sorbent. Purification was performed on a silica gel column (4 g, 0-40% methanol in dichloromethane). The desired fractions were combined and the solvents were removed under reduced pressure to provide the title compound. MS (APCI) m/z 1019.6 (M+H)⁺.

### Example 141E

### (7R,16R)-19,23-dichloro-10-({2-[(1,3-dimethoxypropan-2-yl)oxy]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 141D (29 mg) was dissolved in dichloromethane (3.0 mL) and trifluoroacetic acid (218 μL) was added. The reaction mixture was stirred overnight at room temperature. The reaction mixture was diluted with dichloromethane and aqueous sodium bicarbonate solution (9%). The aqueous layer was extracted with dichloromethane five times. The organic layer was dried over sodium sulfate, filtered, and concentrated. The residue was absorbed on Bulk Isolute Sorbent. Purification was performed on a silica gel column (4 g, 0-100% methanol in dichloromethane). The desired fractions were combined and the solvents were removed under reduced pressure to provide the title compound. ¹H NMR (600 MHz, dimethylsulfoxide-d₆) δ ppm 8.73 (s, 1H), 8.57 (d, 1H), 7.23-7.18 (m, 3H), 7.15-7.13 (m, 2H), 6.83 (d, 1H), 6.75 (dd, 1H), 6.19 (m, 1H), 5.81 (m, 1H), 5.38 (tt, 1H), 5.10 (d, 1H), 5.02 (d, 1H), 4.90-4.87 (m, 1H), 4.46-4.41 (m, 2H), 3.60-3.54 (m, 5H), 3.26 (s, 6H), 2.94 (dd, 1H), 2.70 (dd, 1H), 2.66 (dd, 1H), 2.52-2.28 (m, 8H), 2.18 (s, 3H), 1.98 (s, 3H), 1.96 (s, 3H). MS (APCI) m/z 963.4 (M+H)⁺.

### Example 142

### (7R,16R)-19,23-dichloro-1-(4-fluorophenyl)-20,22-dimethyl-16-[(4-methylpiperazin-1-yl)methyl]-10-[(2-{4-[2-(morpholin-4-yl)ethyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 142A

### (2-(4-(2-morpholinoethyl)phenyl)pyrimidin-4-yl)methanol

The title compound was prepared by substituting (4-(2-morpholinoethyl)phenyl)boronic acid for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.84 (d, 1H), 8.28 (d, 2H), 7.45 (d, 1H), 7.36 (d, 2H), 5.65 (t, 1H), 4.62 (d, 2H), 3.56 (m, 4H), 2.80 (t, 2H), 2.54 (t, 2H), 2.42 (m 4H). MS (ESI) m/z 300.2 (M+H)⁺.

### Example 142B

### (7R,16R)-19,23-dichloro-1-(4-fluorophenyl)-20,22-dimethyl-16-[(4-methylpiperazin-1-yl)methyl]-10-[(2-{4-[2-(morpholin-4-yl)ethyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 142A for Example 38D in Example 38E. ¹H NMR (500 MHz, dimethylsulfoxide-d₆) δ ppm 8.86 (d, 1H), 8.74 (s, 1H), 8.31 (d, 2H), 7.50 (d, 1H), 7.37 (d, 2H), 7.20 (t, 2H), 7.15-7.12 (m, 2H), 6.89 (d, 1H), 6.75 (dd, 1H), 6.26 (m, 1H), 5.81 (d, 1H), 5.24 (q, 2H), 4.85 (m, 1H), 4.45 (m, 2H), 3.67 (dd, 2H), 3.58 (m, 4H), 2.98 (d, 1H), 2.81 (t, 2H), 2.67 (m, 3H), 2.55 (t, 2H), 2.44 (m, 10H), 2.21 (s, 3H), 1.98 (s, 3H), 1.95 (s, 3H). MS (ESI) m/z 1034.2 (M+H)⁺.

### Example 143

### (7R,16R)-19,23-dichloro-1-(4-fluorophenyl)-20,22-dimethyl-16-[(4-methylpiperazin-1-yl)methyl]-10-[(2-{3-[2-(morpholin-4-yl)ethyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 143A

### (2-(3-(2-morpholinoethyl)phenyl)pyrimidin-4-yl)methanol

The title compound was prepared by substituting 4-(3-(4,4,5,5-tetramethyl-1,3,2-dioxaborolan-2-yl) phenethyl)morpholine for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.87 (d, 1H), 8.25 (s, 1H), 8.21 (dt, 1H), 7.48 (d, 1H), 7.44-7.38 (m, 2H), 5.67 (t, 1H), 4.64 (d, 2H), 3.58 (t, 4H), 2.84 (t, 2H), 2.55 (t, 2H), 2.46 (m, 4H). MS (ESI) m/z 300.3 (M+H)⁺.

### Example 143B

### (7R,16R)-19,23-dichloro-1-(4-fluorophenyl)-20,22-dimethyl-16-[(4-methylpiperazin-1-yl)methyl]-10-[(2-{3-[2-(morpholin-4-yl)ethyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 143A for Example 38D in Example 38E. ¹H NMR (500 MHz, dimethylsulfoxide-d₆) δ ppm 8.80 (d, 1H), 8.66 (s, 1H), 8.19 (s, 1H), 8.16 (dt, 1H), 7.45 (d, 1H), 7.38-7.31 (m, 2H), 7.13 (t, 2H), 7.08-7.03 (m, 2H), 6.80 (d, 1H), 6.66 (dd, 1H), 6.16 (m, 1H), 5.77 (d, 1H), 5.17 (q, 2H), 4.80 (m, 1H), 4.37 (m, 2H), 3.59 (dd, 2H), 3.52 (t, 4H), 2.92 (d, 1H), 2.78 (m, 2H), 2.61 (m, 3H), 2.50 (t, 2H), 2.40 (m, 4H), 2.32 (m, 6H), 2.11 (s, 3H), 1.90 (s, 6H). MS (ESI) m/z 1034.3 (M+H)⁺.

### Example 144

### (7R,16R)-19,23-dichloro-1-(4-fluorophenyl)-20,22-dimethyl-10-({2-[4-methyl-4-(morpholin-4-yl)piperidin-1-yl]pyrimidin-4-yl}methoxy)-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 144A

### 4-(4-methylpiperidin-4-yl)morpholine

To a solution of tert-butyl 4-methyl-4-morpholinopiperidine-1-carboxylate (180 mg) in dichloromethane (1.2 mL) at ambient temperature was added trifluoroacetic acid (600 μL), and the reaction mixture was allowed to stand for 2 hours. The reaction mixture was concentrated and used directly in the next step without further purification.

### Example 144B

### (2-(4-methyl-4-morpholinopiperidin-1-yl)pyrimidin-4-yl)methanol

A solution of Example 144A (246 mg), (2-chloropyrimidin-4-yl)methanol (72 mg) and N,N-diisopropylethylamine (440 μL) in acetonitrile (1.2 mL) was heated to 80° C. for 2.5 hours. The reaction was cooled and concentrated. The residue was purified by normal phase MPLC on a Teledyne Isco Combiflash® Rf+12 g gold silica gel column eluting with 0-7% methanol in dichloromethane. Desired fractions were combined, concentrated, taken up in dimethylsulfoxide and purified by RP-HPLC on a Gilson PLC 2020 using a Luna™® column (250×50 mm, 10 mm) (5-55% over 30 minutes with acetonitrile in water containing 0.01% trifluoroacetic acid). Desired fractions were combined, neutralized with saturated aqueous sodium bicarbonate and extracted with dichloromethane three times. The combined organic layers were dried over anhydrous sodium sulfate, filtered and concentrated to give the title compound. ¹H NMR (400 MHz, dimethylsulfoxide-d₆) δ ppm 8.30 (d, 1H), 6.67 (d, 1H), 5.36 (t, 1H), 4.33 (d, 2H), 3.93-3.79 (m, 2H), 3.65-3.49 (m, 5H), 2.48-2.40 (m, 4H), 1.80-1.68 (m, 2H), 1.43-1.28 (m, 2H), 0.91 (s, 3H).

### Example 144C

### tert-butyl (7R,16R)-19,23-dichloro-1-(4-fluorophenyl)-20,22-dimethyl-10-({2-[4-methyl-4-(morpholin-4-yl)piperidin-1-yl]pyrimidin-4-yl}methoxy)-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (25 mg), Example 144B (14 mg) and triphenylphosphine (24 mg) in toluene (80 μL) and tetrahydrofuran (80 μL) was added N,N,N′,N′-tetramethylazodicarboxamide (16 mg), and the reaction was allowed to stir at 50° C. for 3 hours. The reaction mixture was cooled, diluted with ethyl acetate, filtered over diatomaceous earth and concentrated. The residue was purified by normal phase MPLC on a Teledyne Isco Combiflash® Rf+4 g gold silica gel column eluting with 0.5-10% methanol in dichloromethane to give the title compound. ¹H NMR (400 MHz, dimethylsulfoxide-d₆) δ ppm 8.74 (s, 1H), 8.32 (d, 1H), 7.27-7.13 (m, 4H), 6.98-6.77 (m, 2H), 6.67 (d, 1H), 6.06-5.98 (m, 1H), 5.70-5.62 (m, 1H), 5.01-4.82 (m, 2H), 4.79-4.68 (m, 1H), 4.52-4.33 (m, 3H), 3.95-3.83 (m, 2H), 3.70-3.49 (m, 6H), 2.91-2.81 (m, 1H), 2.73-2.59 (m, 2H), 2.51-2.20 (m, 8H), 2.14 (s, 3H), 2.09 (s, 3H), 1.89 (s, 3H), 1.81-1.68 (s, 3H), 1.42-1.29 (m, 2H), 1.06 (s, 9H), 0.91 (s, 3H).

### Example 144D

### (7R,16R)-19,23-dichloro-1-(4-fluorophenyl)-20,22-dimethyl-10-({2-[4-methyl-4-(morpholin-4-yl)piperidin-1-yl]pyrimidin-4-yl}methoxy)-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 144C (24 mg) in dichloromethane (110 μL) was added trifluoroacetic acid (110 μL), and the reaction was allowed to stir for 5 hours. The reaction was concentrated under a stream of nitrogen and taken up in water and acetonitrile. The mixture was purified by RP-HPLC on a Gilson PLC 2020 using a Luna™ column (250×50 mm, 10 mm, 5-75% over 30 minutes with acetonitrile in water containing 10 mM ammonium acetate) to give the title compound. ¹H NMR (400 MHz, dimethylsulfoxide-d₆) δ ppm 8.70 (s, 1H), 8.28 (d, 1H), 7.24-7.06 (m, 4H), 6.77 (d, 1H), 6.73-6.64 (m, 2H), 6.19-6.10 (m, 1H), 5.90-5.82 (m, 1H), 5.00-4.81 (m, 3H), 4.51-4.35 (m, 2H), 3.93-3.81 (m, 2H), 3.63-3.46 (m, 10H), 2.97-2.86 (m, 1H), 2.75-2.59 (m, 3H), 2.54-2.29 (m, 8H), 2.19 (s, 3H), 1.99 (s, 3H), 1.93 (s, 3H), 1.80-1.66 (m, 2H), 1.42-1.28 (m, 2H), 0.90 (s, 3H). MS (ESI) m/z 1025.0 (M−H)⁻.

### Example 145

### (7R,16R)-19,23-dichloro-1-(4-fluorophenyl)-20,22-dimethyl-16-[(4-methylpiperazin-1-yl)methyl]-10-({2-[4-(morpholine-4-sulfonyl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 145A

### (2-(4-(morpholinosulfonyl)phenyl)pyrimidin-4-yl)methanol

The title compound was prepared by substituting (4-(morpholinosulfonyl)phenyl)boronic acid for tert-butyl 2-(4,4,5,5-tetramethyl-1,3,2-dioxaborolan-2-yl)benzoate and (2-chloropyrimidin-4-yl)methanol for (2-bromopyrimidin-4-yl)methanol in Example 19A. ¹H NMR (500 MHz, dimethylsulfoxide-d₆) δ ppm 8.97 (d, 1H), 8.63 (d, 2H), 7.90 (d, 2H), 7.60 (d, 1H), 5.75 (t, 1H), 4.68 (d, 2H), 3.64 (t, 4H), 2.92 (t, 4H). MS (ESI) m/z 336.1 (M+H)⁺.

### Example 145B

### (7R,16R)-19,23-dichloro-1-(4-fluorophenyl)-20,22-dimethyl-16-[(4-methylpiperazin-1-yl)methyl]-10-({2-[4-(morpholine-4-sulfonyl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 145A for Example 13C in Example 160. ¹H NMR (500 MHz, dimethylsulfoxide-d₆) δ ppm 8.90 (d, 1H), 8.66 (s, 1H), 8.57 (d, 2H), 7.84 (d, 2H), 7.57 (d, 1H), 7.15-7.05 (m, 4H), 6.82 (d, 1H), 6.67 (dd, 1H), 6.16 (m, 1H), 5.75 (d, 1H), 5.21 (q, 2H), 4.78 (m, 1H), 4.38 (m, 2H), 3.62-3.56 (m, 6H), 2.92 (dd, 2H), 2.86 (m, 4H), 2.60 (m, 2H), 2.40-2.24 (m, 6H), 2.08 (s, 3H), 1.92 (s, 3H), 1.89 (s, 3H). MS (ESI) m/z 1070.5 (M+H)⁺.

### Example 146

### (7R,16R)-19,23-dichloro-1-(4-fluorophenyl)-10-[(2-{[(3R,3aR,6R,6aR)-6-methoxyhexahydrofuro[3,2-b]furan-3-yl]oxy}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 146A

### 4-(((tert-butyldimethylsilyl)oxy)methyl)-2-(((3R,3aR,6R,6aR)-6-methoxyhexahydrofuro[3,2-b]furan-3-yl)oxy)pyrimidine

To a solution of Example 133A (136 mg) and Example 38A (200 mg) in acetonitrile (2.6 mL) at ambient temperature was added sodium hydride (93 mg, 60% oil dispersion), and the reaction was allowed to stir overnight. The reaction mixture was diluted with saturated aqueous ammonium chloride and water and extracted with ethyl acetate three times. The combined organic layers were dried over anhydrous sodium sulfate, filtered and concentrated. The residue was purified by normal phase MPLC on a Teledyne Isco Combiflash® Rf+24 g gold silica gel column eluting with 25-100% ethyl acetate in heptanes to give the title compound. ¹H NMR (500 MHz, dimethylsulfoxide-d₆) δ ppm 8.59 (d, 1H), 7.14 (d, 1H), 5.31-5.23 (m, 1H), 4.82-4.76 (m, 1H), 4.72-4.61 (m, 2H), 4.60-4.54 (m, 1H), 4.11-4.02 (m, 1H), 3.92-3.74 (m, 3H), 3.47-3.39 (m, 1H), 3.33 (s, 3H), 0.92 (s, 9H), 0.10 (s, 6H).

### Example 146B

### (2-(((3R,3aR,6R,6aR)-6-methoxyhexahydrofuro[3,2-b]furan-3-yl)oxy)pyrimidin-4-yl)methanol

To a solution of Example 146A (120 mg) in tetrahydrofuran (1 mL) and methanol (500 μL) was added cesium fluoride (240 mg), and the reaction was allowed to stir for 3 hours. The reaction was concentrated, and the residue was taken up in ethyl acetate, filtered over diatomaceous earth and concentrated. The residue was purified by normal phase MPLC on a Teledyne Isco Combiflash® Rf+12 g gold silica gel column eluting with 0-8% methanol in dichloromethane to give the title compound. ¹H NMR (400 MHz, dimethylsulfoxide-d₆) δ ppm 8.55 (d, 1H), 7.19 (d, 1H), 5.64-5.56 (m, 1H), 5.32-5.23 (m, 1H), 4.83-4.75 (m, 1H), 4.62-4.54 (m, 1H), 4.50-4.43 (m, 2H), 4.11-4.02 (m, 1H), 3.93-3.73 (m, 3H), 3.48-3.39 (m, 1H), 3.33 (s, 3H).

### Example 146C

### tert-butyl (7R,16R)-19,23-dichloro-1-(4-fluorophenyl)-10-[(2-{[(3R,3aR,6R,6aR)-6-methoxyhexahydrofuro[3,2-b]furan-3-yl]oxy}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A vial containing Example 146B (35 mg), Example 16N (35 mg), triphenylphosphine (34 mg) and N,N,N′,N′-tetramethylazodicarboxamide (22 mg) in toluene (110 μL) and tetrahydrofuran (110 μL) was allowed to stir at 50° C. for 5 hours. The reaction was cooled, diluted with ethyl acetate, filtered over diatomaceous earth and concentrated. The residue was purified by normal phase MPLC on a Teledyne Isco Combiflash® Rf+4 g gold silica gel column eluting with 0.5-9% methanol in dichloromethane to give the title compound.

### Example 146D

### (7R,16R)-19,23-dichloro-1-(4-fluorophenyl)-10-[(2-{[(3R,3aR,6R,6aR)-6-methoxyhexahydrofuro[3,2-b]furan-3-yl]oxy}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 146C (46 mg) in dichloromethane (220 μL) was added trifluoroacetic acid (220 μL), and the reaction was allowed to stir for 4 hours. The reaction was concentrated under a stream of nitrogen and was taken up in water and acetonitrile. The mixture was purified by RP-HPLC on a Gilson PLC 2020 using a Luna™ column (250×50 mm, 10 mm, 5-80% over 30 minutes with acetonitrile in water containing 10 mM ammonium acetate) to give the title compound. ¹H NMR (400 MHz, dimethylsulfoxide-d₆) δ ppm 8.73 (s, 1H), 8.57 (d, 1H), 7.29-7.07 (m, 5H), 6.87-6.79 (d, 1H), 6.78-6.70 (m, 1H), 6.27-6.18 (m, 1H), 5.86-5.77 (m, 1H), 5.35-5.24 (m, 1H), 5.15-4.98 (m, 2H), 4.93-4.76 (m, 2H), 4.61-4.53 (m, 1H), 4.50-4.38 (m, 2H), 4.12-4.03 (m, 1H), 3.93-3.75 (m, 3H), 3.66-3.55 (m, 1H), 3.49-3.39 (m, 1H), 3.33 (s, 3H), 2.99-2.88 (m, 1H), 2.76-2.60 (m, 3H), 2.59-2.40 (m, 6H), 2.26 (s, 3H), 2.02-1.93 (m, 6H). MS (ESI) m/z 1000.8 (M−H)⁻.

### Example 147

### (7R,16R)-19,23-dichloro-1-(4-fluorophenyl)-20,22-dimethyl-16-[(4-methylpiperazin-1-yl)methyl]-10-[(2-{3-[(morpholin-4-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 147A

### (2-(3-(morpholinomethyl)phenyl)pyrimidin-4-yl)methanol

The title compound was prepared by substituting 4-(3-(4,4,5,5-tetramethyl-1,3,2-dioxaborolan-2-yl)benzyl)morpholine hydrochloride for tert-butyl 2-(4,4,5,5-tetramethyl-1,3,2-dioxaborolan-2-yl)benzoate and (2-chloropyrimidin-4-yl)methanol for (2-bromopyrimidin-4-yl)methanol in Example 19A. ¹H NMR (500 MHz, dimethylsulfoxide-d₆) δ ppm 8.88 (d, 1H), 8.33 (bs, 1H), 8.30-8.27 (m, 1H), 7.51-7.45 (m, 3H), 5.68 (t, 1H), 4.65 (d, 2H), 3.55 (bs, 2H), 3.58-3.54 (m, 4H), 2.39 (m, 4H). MS (ESI) m/z 286.3 (M+H)⁺.

### Example 147B

### (7R,16R)-19,23-dichloro-1-(4-fluorophenyl)-20,22-dimethyl-16-[(4-methylpiperazin-1-yl)methyl]-10-[(2-{3-[(morpholin-4-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 147A for Example 13C in Example 160. ¹H NMR (500 MHz, dimethylsulfoxide-d₆) δ ppm 8.89 (d, 1H), 8.73 (s, 1H), 8.36 (s, 1H), 8.30 (m, 1H), 7.53 (d, 1H), 7.48 (d, 2H), 7.22-7.12 (m, 4H), 6.89 (d, 1H), 6.75 (dd, 1H), 6.24 (m, 1H), 5.83 (d, 1H), 5.24 (q, 2H), 4.86 (m, 1H), 4.45 (m, 2H), 3.66 (dd, 2H), 3.57 (m, 4H), 2.99 (d, 2H), 2.67 (m, 2H), 2.46-2.33 (m, 12H), 2.18 (s, 3H), 1.98 (s, 3H), 1.96 (s, 3H). MS (ESI) m/z 1022.4 (M+H)⁺.

### Example 148

### (7R,16R)-19,23-dichloro-1-(4-fluorophenyl)-20,22-dimethyl-16-[(4-methylpiperazin-1-yl)methyl]-10-[(2-{4-[(morpholin-4-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 148A

### (2-(4-(morpholinomethyl)phenyl)pyrimidin-4-yl)methanol

The title compound was prepared by substituting 4-(4-(4,4,5,5-tetramethyl-1,3,2-dioxaborolan-2-yl)benzyl)morpholine for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.87 (d, 1H), 8.34 (d, 2H), 7.48 (d, 1H), 7.45 (d, 2H), 5.67 (t, 1H), 4.64 (d, 2H), 3.59 (t, 4H), 3.53 (s, 2H), 2.38 (m, 4H). MS (ESI) m/z 286.3 (M+H)⁺.

### Example 148B

### (7R,16R)-19,23-dichloro-1-(4-fluorophenyl)-20,22-dimethyl-16-[(4-methylpiperazin-1-yl)methyl]-10-[(2-{4-[(morpholin-4-yl)methyl]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 148A for Example 13C in Example 160. ¹H NMR (500 MHz, dimethylsulfoxide-d₆) δ ppm 8.86 (d, 1H), 8.73 (s, 1H), 8.35 (d, 2H), 7.53 (d, 1H), 7.45 (d, 2H), 7.19 (m, 2H), 7.13 (m, 2H), 6.86 (d, 1H), 6.72 (dd, 1H), 6.19 (m, 1H), 5.86 (d, 1H), 5.22 (q, 2H), 4.87 (m, 1H), 4.44 (m, 2H), 3.65 (dd, 2H), 3.58 (m, 4H), 3.53 (s, 2H), 2.97 (d, 2H), 2.66 (m, 4H), 2.46-2.28 (m, 8H), 2.16 (s, 3H), 1.97 (s, 3H), 1.95 (s, 3H). MS (ESI) m/z 1020.3 (M+H)⁺, 1018.0 (M−H)⁻.

### Example 149

### (7R,16R)-19,23-dichloro-1-(4-fluorophenyl)-20,22-dimethyl-16-[(4-methylpiperazin-1-yl)methyl]-10-({2-[3-(morpholine-4-sulfonyl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 149A

### (2-(3-(morpholinosulfonyl)phenyl)pyrimidin-4-yl)methanol

The title compound was prepared by substituting (3-(morpholinosulfonyl)phenyl)boronic acid for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.96 (d, 1H), 8.72 (dd, 1H), 8.69 (m, 1H), 7.90-7.81 (m, 2H), 7.59 (d, 1H), 5.73 (t, 1H), 4.68 (d, 2H), 3.64 (t, 4H), 2.92 (t, 4H). MS (ESI) m/z 336.3 (M+H)⁺.

### Example 149B

### (7R,16R)-19,23-dichloro-1-(4-fluorophenyl)-20,22-dimethyl-16-[(4-methylpiperazin-1-yl)methyl]-10-({2-[3-(morpholine-4-sulfonyl)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 149A for Example 13C in Example 160. ¹H NMR (500 MHz, dimethylsulfoxide-d₆) δ ppm 8.92 (d, 1H), 8.69-8.65 (m, 3H), 7.86-7.74 (m, 2H), 7.57 (d, 1H), 7.15-7.06 (m, 4H), 6.90 (d, 1H), 6.75 (dd, 1H), 6.22 (m, 1H), 5.72 (d, 1H), 5.22 (q, 2H), 4.83 (m, 1H), 4.39 (m, 2H), 3.65-3.55 (m, 6H), 3.06-2.93 (m, 6H), 2.85 (m, 4H), 2.73 (m, 4H), 2.60 (m, 3H), 1.92 (s, 3H), 1.88 (s, 3H). MS (ESI) m/z 1070.4 (M+H)⁺.

### Example 150

### (7R,16R)-19,23-dichloro-1-(4-fluorophenyl)-10-({2-[(3S,8aS)-hexahydro-1H-pyrrolo[2,1-c][1,4]oxazin-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 150A

### tert-butyl (7R,16R)-19,23-dichloro-1-(4-fluorophenyl)-10-({2-[(3S,8aS)-hexahydro-1H-pyrrolo[2,1-c][1.4]oxazin-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (30 mg), (2-((3S,8aS)-hexahydro-1H-pyrrolo[2,1-c][1.4]oxazin-3-yl)pyrimidin-4-yl)methanol (25 mg, commercially available from Chemspace (CAS 1502498-81-8)), triphenylphosphine (30 mg) and (E)-N¹,N¹,N²,N²-tetramethyldiazene-1,2-dicarboxamide (TMAD) (20 mg). The mixture was purged for 30 minutes with argon. A mixture of toluene (0.5 mL) and tetrahydrofuran (0.5 mL) was added and the reaction mixture was stirred overnight at ambient temperature. The reaction mixture was then concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-100% methanol in dichloromethane) to afford the title compound. MS (APCI) m/z 1026.55 (M+H)⁺.

### Example 150B

### (7R,16R)-19,23-dichloro-1-(4-fluorophenyl)-10-({2-[(3S,8aS)-hexahydro-1H-pyrrolo[2,1-c][1.4]oxazin-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 150A (26 mg) in dichloromethane (1 mL) was added trifluoroacetic acid (150 μL). The reaction mixture was stirred for 20 hours at ambient temperature. The reaction mixture was then concentrated in vacuo. The residue was purified by HPLC (Waters X-Bridge C18 19×150 mm 5 μm column, gradient 5-95% acetonitrile+0.1% trifluoroacetic acid in water+0.1% trifluoroacetic acid) to provide the title compound as a trifluoroacetic acid salt. The residue was dissolved in dichloromethane (5 mL) and saturated aqueous NaHCO₃-solution was added. The reaction mixture was stirred for 30 minutes at ambient temperature. The phases were separated with a Horizon DryDisk® and the organic phase was concentrated in vacuo to provide the title compound. ¹H NMR (600 MHz, dimethylsulfoxide-d₆) δ ppm 8.79 (d, 1H), 8.75 (s, 1H), 7.53 (d, 1H), 7.21 (m, 2H), 7.15 (m, 2H), 6.86 (d, 1H), 6.77 (m, 1H), 6.23 (m, 1H), 5.77 (m, 1H), 5.20 (d, 1H), 5.15 (d, 1H), 4.86 (m, 1H), 4.45 (m, 2H), 4.03 (m, 1H), 3.63 (m, 1H), 3.25 (m, 2H), 3.04 (m, 1H), 2.96 (m, 1H), 2.68 (m, 2H), 2.50-2.25 (m, 9H), 2.19 (s, 3H), 2.13 (m, 1H), 2.09 (m, 1H), 1.97 (s, 3H), 1.94 (s, 3H), 1.71 (m, 3H), 1.28 (m, 1H). MS (APCI) m/z 970.4 (M+H)⁺.

### Example 151

### (7R,16R)-19,23-dichloro-1-(4-fluorophenyl)-20,22-dimethyl-16-[(4-methylpiperazin-1-yl)methyl]-10-[(2-{4-[(morpholine-4-carbonyl)oxy]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 151A

### 4-(4,4,5,5-tetramethyl-1,3,2-dioxaborolan-2-yl)phenyl morpholine-4-carboxylate

4-Hydroxyphenylboronic acid pinacol ester (103 mg) was dissolved under nitrogen atmosphere in dichloromethane. 4-Dimethylaminopyridine (150 mg) and 4-morpholinecarbonyl chloride (0.12 mL) were added. The reaction mixture was stirred overnight at ambient temperature. The reaction mixture was diluted with ethyl acetate. The organic layer was washed with water thrice, dried over magnesium sulfate, filtrated, and concentrated. Purification of the residue was performed on a silica gel column (4 g, 0-5% methanol in dichloromethane). The desired fractions were combined and the solvents were removed under reduced pressure to provide the title compound. MS (ESI) m/z 334.2 (M+H)⁺.

### Example 151B

### 4-(4-(hydroxymethyl)pyrimidin-2-yl)phenyl morpholine-4-carboxylate

Example 151A (56 mg), (2-chloropyrimidin-4-yl)methanol (25 mg), and tetrakis(triphenylphosphine)palladium (1.94 mg) were combined in tetrahydrofuran (2.5 mL). Aqueous sodium bicarbonate solution (1.0 mL, 90%) was added under argon. The reaction mixture was degassed with argon for 5 minutes and then heated at 120° C. in a Biotage® Initiator microwave for 2 hours. The reaction mixture was partitioned between water and ethyl acetate. The aqueous layer was extracted with ethyl acetate twice. The combined organic layers were dried over magnesium sulfate, filtrated and concentrated. Purification of the residue was performed on a silica gel column (4 g, 0-5% methanol in dichloromethane). The desired fractions were combined and the solvents were removed under reduced pressure to provide the title compound. MS (ESI) m/z 316.1 (M+H)⁺.

### Example 151C

### tert-butyl (7R,16R)-19,23-dichloro-1-(4-fluorophenyl)-20,22-dimethyl-16-[(4-methylpiperazin-1-yl)methyl]-10-[(2-{4-[(morpholine-4-carbonyl)oxy]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylate

Example 16N (30 mg), Example 151B (22 mg), triphenylphosphine (37 mg), and N,N,N′,N′-tetramethylazodicarboxamide (28 mg) were combined under an argon atmosphere. Tetrahydrofuran (0.6 mL) and toluene (0.6 mL) were added. The reaction mixture was stirred overnight at ambient temperature. All volatiles were removed in vacuo and the residue was partitioned between dichloromethane and aqueous saturated sodium bicarbonate solution. The aqueous layer was extracted twice with dichloromethane. The combined organic extracts were dried over magnesium sulfate, filtrated and concentrated. Purification was performed on a silica gel column (4 g, 0-8% methanol in dichloromethane). The desired fractions were combined and the solvents were removed under reduced pressure to provide the title compound. MS (APCI) m/z 1106.6 (M+H)⁺.

### Example 151D

### (7R,16R)-19,23-dichloro-1-(4-fluorophenyl)-20,22-dimethyl-16-[(4-methylpiperazin-1-yl)methyl]-10-[(2-{4-[(morpholine-4-carbonyl)oxy]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 151C (36 mg) was dissolved in dichloromethane (1 mL), trifluoroacetic acid (250 μL, 3.24 mmol) was added, and the mixture was stirred overnight at ambient temperature. The reaction mixture was diluted with dichloromethane and washed with aqueous sodium bicarbonate solution. The separated aqueous layer was extracted with dichloromethane, dried over magnesium sulfate, filtrated, and concentrated. The crude material was purified by HPLC (Waters XSelect CSH C18 19×150 mm 5 μm column, gradient 5-100% acetonitrile+0.1% trifluoroacetic acid in water+0.1% trifluoroacetic acid). The residue was dissolved in dichloromethane, and washed with aqueous saturated sodium bicarbonate solution. The separated aqueous layer (pH 9) was extracted with dichloromethane another two times. The combined dichloromethane extracts were dried over magnesium sulfate, filtrated, and concentrated to yield the title compound. ¹H NMR (600 MHz, dimethylsulfoxide-d₆) δ ppm 8.89 (d, 1H), 8.75 (s, 1H), 8.43-8.41 (m, 2H), 7.52 (d, 1H), 7.31-7.29 (m, 2H), 7.22-7.18 (m, 2H), 7.16-7.13 (m, 2H), 6.90 (d, 1H), 6.77 (dd, 1H), 6.25 (b, 1H), 5.79 (b, 1H), 5.28 (d, 1H), 5.20 (d, 1H), 4.87-4.84 (m, 1H), 4.47-4.42 (m, 2H), 3.67-3.62 (m, 5H), 3.62 (b, 2H), 3.44 (b, 2H), 2.99 (dd, 1H), 2.67 (qd, 2H), 2.52-2.30 (m, 8H), 2.17 (s, 3H), 2.00 (s, 3H), 1.95 (s, 3H). MS (APCI) m/z 1050.3 (M+H)⁺.

### Example 152

### (7R,16R)-10-({2-[3,4-bis(2,5,8,11-tetraoxadodecan-1-yl)phenyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 152A

### 1,1′-(4-bromo-1,2-phenylene)bis(2,5,8,11-tetraoxadodecane)

4-Bromo-1,2-bis(bromomethyl)benzene(250 mg) and 2-(2-(2-methoxyethoxy)ethoxy)ethanol (263 mg) were dissolved in dioxane (8 mL). Sodium hydride (60%, 64.2 mg) was added, and the solution was mixed at ambient temperature. After 20 minutes, the solvent was removed under vacuum. The residue was suspended in ethyl acetate (20 mL), washed with brine (5 mL) and dried over anhydrous sodium sulfate. The solution was concentrated and syringe filtered. The remaining solvent was then removed under vacuum, and the material was utilized without further purification. ¹H NMR (500 MHz, dimethylsulfoxide-d₆) δ ppm 7.57 (d, 1H), 7.48 (dd, 1H), 7.33 (d, 1H), 4.54 (s, 2H), 4.50 (s, 2H), 3.60-3.55 (m, 8H), 3.53-3.50 (m, 12H), 3.44-3.40 (m, 4H), 3.24-3.23 (m, 6H). MS (ESI) m/z 526.2 (M+H)⁺.

### Example 152B

### 2-(3,4-di(2,5,8,11-tetraoxadodecyl)phenyl)-4,4,5,5-tetramethyl-1,3,2-dioxaborolane

The title compound was prepared by substituting Example 152A for 1-bromo-3-(2-(2-(2-methoxyethoxy)ethoxy)ethoxy)benzene in Example 104B. ¹H NMR (500 MHz, dimethylsulfoxide-d₆) o ppm 7.67 (d, 1H), 7.59 (dd, 1H), 7.41 (d, 1H), 4.58 (s, 2H), 4.54 (s, 2H), 3.55 (m, 8H), 3.51 (m, 12H), 3.42 (m, 4H), 3.24-3.22 (m, 6H), 1.29 (s, 12H). MS (ESI) m/z 574.3 (M+NH₄)⁺.

### Example 152C

### (2-(3,4-di(2,5,8,11-tetraoxadodecyl)phenyl)pyrimidin-4-yl)methanol

The title compound was prepared by substituting Example 152B for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.87 (d, 1H), 8.41 (d, 1H), 8.30 (dd, 1H), 7.53 (d, 1H), 7.49 (d, 1H), 5.67 (t, 1H), 4.66-4.62 (m, 6H), 3.59 (m, 8H), 3.52 (m, 12H), 3.42 (m, 4H), 3.24-3.21 (m, 6H). MS (ESI) m/z 539.5 (M+H)⁺.

### Example 152D

### (7R,16R)-10-({2-[3,4-bis(2,5,8,11-tetraoxadodecan-1-yl)phenyl]pyrimidin-4-yl}methoxy)-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 152C for Example 13C in Example 160. ¹H NMR (500 MHz, dimethylsulfoxide-d₆) δ ppm 8.81 (d, 1H), 8.67 (s, 1H), 8.36 (d, 1H), 8.25 (dd, 1H), 7.47 (dd, 2H), 7.13 (t, 2H), 7.07 (dd, 2H), 6.84 (d, 1H), 6.69 (dd, 1H), 6.19 (dd, 1H), 5.74 (d, 1H), 5.17 (q, 2H), 4.79 (t, 1H), 4.56 (bs, 4H), 4.38 (d, 2H), 3.60 (dd, 1H), 3.53 (m, 8H), 3.47-3.42 (m, 12H), 3.37-3.31 (m, 6H), 3.16 (s, 36H), 3.13 (s, 3H), 2.92 (d, 1H), 2.60 (m, 2H), 2.39-2.25 (m, 6H), 2.11 (s, 3H), 1.92 (s, 3H), 1.88 (s, 3H). MS (ESI) m/z 1273.4 (M+H)⁺.

### Example 153

### (7R,16R)-19,23-dichloro-1-(4-fluorophenyl)-10-({2-[2-{2-[2-(2-methoxyethoxy)ethoxy]ethoxy}-4-(2,5,8,11-tetraoxadodecan-1-yl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 153A

### 2-bromo-5-(((tert-butyldimethylsilyl)oxy)methyl)phenol

2-Bromo-5-(hydroxymethyl)phenol (2 g) was taken up in tetrahydrofuran (24 mL). 1H-Imidazole (1.475 g) was added, and the mixture was cooled to 0° C. tert-Butylchlorodimethylsilane (1.633 g) dissolved in tetrahydrofuran (12 mL) was added. The mixture was stirred at 0° C. for five minutes and then allowed to warm to ambient temperature. Additional tetrahydrofuran (18 mL) was added. The mixture was stirred overnight at ambient temperature. Saturated aqueous ammonium chloride (10 mL) was added, and the mixture was extracted with ethyl acetate (20 mL) twice. The organic extracts were combined, washed with water and washed with brine. The organics were dried on anhydrous sodium sulfate, filtered, and concentrated. The material was purified by flash column chromatography on silica gel using a gradient of 5-10% ethyl acetate in heptanes. The solvent was removed from the desired fractions under vacuum to yield the title compound. ¹H NMR (500 MHz, dimethylsulfoxide-d₆) δ ppm 10.15 (bs, 1H), 7.39 (d, 1H), 6.93 (d, 1H), 6.64 (dd, 1H), 4.59 (s, 2H), 0.89 (s, 9H). MS (ESI) m/z 315.0 (M−H)⁻.

### Example 153B

### ((4-bromo-3-(2-(2-(2-methoxyethoxy)ethoxy)ethoxy)benzyl)oxy) (tert-butyl)dimethylsilane

Example 153A (400 mg) and 2-(2-(2-methoxyethoxy)ethoxy)ethanol (251 mg) were taken up in tetrahydrofuran (6 mL). Triphenylphosphine (496 mg) was added, followed by (E)-diisopropyl diazene-1,2-dicarboxylate (382 mg). The mixture was stirred overnight at ambient temperature. The mixture was concentrated on vacuum and purified by flash column chromatography on silica gel using a gradient of 30-100% ethyl acetate in heptanes. The solvent was removed under vacuum to yield the title compound. ¹H NMR (500 MHz, dimethylsulfoxide-d₆) δ ppm 7.51 (d, 1H), 7.03 (d, 1H), 6.84 (dd, 1H), 4.66 (s, 2H), 4.13 (t, 2H), 3.77 (t, 2H), 3.62 (m, 2H), 3.52 (m, 4H), 3.40 (m, 2H), 3.21 (s, 3H), 0.89 (s, 9H). MS (ESI) m/z 480.2 (M+NH₄)⁺.

### Example 153C

### (4-bromo-3-(2-(2-(2-methoxyethoxy)ethoxy)ethoxy)phenyl)methanol

Example 153B (517 mg) was taken up in tetrahydrofuran (4 mL). Tetrabutylammonium fluoride (1 M in tetrahydrofuran, 3.35 mL) was added, and the mixture was stirred at ambient temperature for 30 minutes. The mixture was concentrated on vacuum and purified by flash column chromatography on silica gel using ethyl acetate. The solvent was removed on vacuum to yield the title compound. ¹H NMR (500 MHz, dimethylsulfoxide-d₆) δ ppm 7.49 (d, 1H), 7.07 (d, 1H), 6.85 (dd, 1H), 5.26 (t, 1H), 4.46 (d, 2H), 4.15 (t, 2H), 3.78 (t, 2H), 3.64 (m, 2H), 3.54 (m, 4H), 3.42 (m, 2H), 3.23 (s, 3H). MS (ESI) m/z 366.1 (M+NH₄)⁺.

### Example 153D

### 1-(4-bromo-3-(2-(2-(2-methoxyethoxy)ethoxy)ethoxy)phenyl)-2,5,8,11-tetraoxadodecane

Example 153C (175 mg) and 2-(2-(2-methoxyethoxy)ethoxy)ethyl methanesulfonate (243 mg) were taken up in 1,4-dioxane (6 mL). Sodium hydride (60%, 13.8 mg) was added, and the mixture was stirred at ambient temperature for five minutes. Additional sodium hydride (60%, 13.8 mg) was added, and the mixture was heated to 50° C. for one hour. The mixture was cooled and concentrated on vacuum. The material was purified by flash column chromatography on silica gel using a gradient of 0-5% methanol in ethyl acetate. The solvent was removed from the desired fractions on vacuum to yield the title compound. ¹H NMR (500 MHz, dimethylsulfoxide-d₆) δ ppm 7.52 (d, 1H), 7.06 (d, 1H), 6.85 (dd, 1H), 4.45 (s, 2H), 4.15 (t, 2H), 3.77 (t, 2H), 3.62 (m, 2H), 3.54-3.48 (m, 14H), 3.41 (m, 4H), 3.22 (s, 6H). MS (ESI) m/z 512.2 (M+NH₄)⁺.

### Example 153E

### 2-(4-(2,5,8,11-tetraoxadodecyl)-2-(2-(2-(2-methoxyethoxy)ethoxy)ethoxy)phenyl)-4,4,5,5-tetramethyl-1,3,2-dioxaborolane

The title compound was prepared by substituting Example 153D for 1-bromo-3-(2-(2-(2-methoxyethoxy)ethoxy)ethoxy)benzene in Example 104B. ¹H NMR (500 MHz, dimethylsulfoxide-d₆) δ ppm 7.46 (d, 1H), 6.89 (d, 1H), 6.85 (dd, 1H), 4.49 (s, 2H), 4.04 (t, 2H), 3.74 (m, 4H), 3.69 (m, 2H), 3.59-3.50 (m, 10H), 3.42 (m, 6H), 3.23-3.22 (m, 6H), 1.26 (bs, 12H). MS (ESI) m/z 560.0 (M+NH₄)⁺.

### Example 153F

### (2-(4-(2,5,8,11-tetraoxadodecyl)-2-(2-(2-(2-methoxyethoxy)ethoxy)ethoxy)phenyl)pyrimidin-4-yl)methanol

The title compound was prepared by substituting Example 153E for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.83 (d, 1H), 8.68 (d, 1H), 7.62 (d, 1H), 7.10 (d, 1H), 7.02 (dd, 1H), 5.62 (t, 1H), 4.58 (d, 2H), 4.55 (s, 2H), 4.11 (t, 2H), 3.73 (m, 2H), 3.67 (m, 2H), 3.60-3.57 (m, 4H), 3.56-3.49 (m, 8H), 3.47-3.38 (m, 6H), 3.24-3.22 (m, 6H). MS (ESI) m/z 525.2 (M+H)⁺.

### Example 153G

### (7R,16R)-19,23-dichloro-1-(4-fluorophenyl)-10-({2-[2-{2-[2-(2-methoxyethoxy)ethoxy]ethoxy}-4-(2,5,8,11-tetraoxadodecan-1-yl)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 153F for Example 13C in Example 160. ¹H NMR (500 MHz, dimethylsulfoxide-d₆) δ ppm 8.85 (d, 1H), 8.74 (s, 1H), 7.65-7.47 (m, 2H), 7.20 (t, 2H), 7.16-7.11 (m, 3H), 7.03 (d, 1H), 6.87 (d, 1H), 6.76 (dd, 1H), 6.24 (d, 1H), 5.82 (d, 1H), 5.17 (q, 2H), 4.88 (m, 1H), 4.56 (s, 2H), 4.45 (d, 2H), 4.13 (t, 2H), 3.69 (t, 2H), 3.66-3.58 (m, 6H), 3.57-3.48 (m, 8H), 3.43 (m, 6H), 3.36-3.33 (m, 4H), 3.23 (s, 3H), 3.18 (s, 3H), 2.98 (d, 1H), 2.69 (m, 2H), 2.45 (m, 2H), 2.38 (m, 3H), 2.18 (s, 3H), 1.99 (s, 3H), 1.97 (s, 3H). MS (ESI) m/z 1259.6 (M+H)⁺.

### Example 154

### (7R,16R)-19,23-dichloro-1-(4-fluorophenyl)-10-({2-[4-(2-{[(3R,3aR,6R,6aR)-6-methoxyhexahydrofuro[3,2-b]furan-3-yl]oxy}ethoxy)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 154A

### 2-(4-(2-(((3R,3aR,6R,6aR)-6-methoxyhexahydrofuro[3,2-b]furan-3-yl)oxy)ethoxy)phenyl)-4,4,5,5-tetramethyl-1,3,2-dioxaborolane

To a solution of 4-(4,4,5,5-tetramethyl-1,3,2-dioxaborolan-2-yl)phenol (250 mg), Example 134B (290 mg) and triphenylphosphine (450 mg) in tetrahydrofuran (3.4 mL) at ambient temperature was added di-tert-butyl azodicarboxylate (390 mg), and the reaction was allowed to stir overnight. The reaction was concentrated, and the residue was purified by normal phase MPLC on a Teledyne Isco Combiflash® Rf+24 g gold silica gel column eluting with 20-100% ethyl acetate in heptanes to give the title compound. ¹H NMR (500 MHz, dimethylsulfoxide-d₆) δ ppm 7.63-7.55 (m, 2H), 6.98-6.89 (m, 2H), 4.56-4.50 (m, 2H), 4.14-4.05 (m, 3H), 3.93-3.84 (m, 4H), 3.80-3.73 (m, 1H), 3.47-3.40 (m, 2H), 3.30 (s, 3H), 1.27 (s, 12H).

### Example 154B

### (2-(4-(2-(((3R,3aR,6R,6aR)-6-methoxyhexahydrofuro[3,2-b]furan-3-yl)oxy)ethoxy)phenyl)pyrimidin-4-yl)methanol

To a solution of Example 154A (200 mg) and (2-chloropyrimidin-4-yl)methanol (70 mg) in tetrahydrofuran (2.1 mL) and saturated sodium bicarbonate (1.2 mL) was added tetrakis(triphenylphosphine)palladium (0) (57 mg), and the reaction was purged with nitrogen and heated to 75° C. overnight. The reaction was cooled, diluted with ethyl acetate and water, and the aqueous layer was extracted three times with ethyl acetate. The combined organic layers were dried over anhydrous sodium sulfate, filtered and concentrated. The residue was purified by normal phase MPLC on a Teledyne Isco Combiflash® Rf+12 g gold silica gel column eluting with 60-100% ethyl acetate in heptanes. Desired fractions were combined and concentrated, and the residue was purified by normal phase MPLC on a Teledyne Isco Combiflash® Rf+12 g gold silica gel column eluting with 0-4% methanol in dichloromethane to give the title compound. ¹H NMR (400 MHz, dimethylsulfoxide-d₆) ppm 8.81 (d, 1H), 8.37-8.28 (m, 2H), 7.40 (d, 1H), 7.11-7.01 (m, 2H), 5.68-5.60 (m, 1H), 4.61 (d, 2H), 4.58-4.49 (m, 2H), 4.22-4.05 (m, 3H), 3.97-3.74 (m, 5H), 3.51-3.39 (m, 2H), 3.30 (s, 3H).

### Example 154C

### tert-butyl (7R,16R)-19,23-dichloro-1-(4-fluorophenyl)-10-({2-[4-(2-{[(3R,3aR,6R,6aR)-6-methoxyhexahydrofuro[3,2-b]furan-3-yl]oxy}ethoxy)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 154B (59 mg) and Example 16N (41 mg) in tetrahydrofuran (250 μL) and toluene (250 μL) was added triphenylphosphine (40 mg) followed by N,N,N′,N′-tetramethylazodicarboxamide (26 mg), and the reaction was allowed to stir at 50° C. for 4 hours. The reaction was cooled, diluted with ethyl acetate, filtered over diatomaceous earth and concentrated. The residue was purified by normal phase MPLC on a Teledyne Isco Combiflash® Rf+4 g gold silica gel column eluting with 0-8% methanol in dichloromethane to give the title compound.

### Example 154D

### (7R,16R)-19,23-dichloro-1-(4-fluorophenyl)-10-({2-[4-(2-{[(3R,3aR,6R,6aR)-6-methoxyhexahydrofuro[3,2-b]furan-3-yl]oxy}ethoxy)phenyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 154C (56 mg) in dichloromethane (240 μL) was added trifluoroacetic acid (240 μL), and the reaction was allowed to stir overnight. The reaction was concentrated under a stream of nitrogen and taken up in water and acetonitrile. The mixture was purified by RP-HPLC on a Gilson PLC 2020 using a Luna™ column (250×50 mm, 10 mm, 30-80% over 30 minutes with acetonitrile in water containing 10 mM ammonium acetate) to give the title compound. ¹H NMR (400 MHz, dimethylsulfoxide-d₆) δ ppm 8.80 (d, 1H), 8.72 (s, 1H), 8.38-8.28 (m, 2H), 7.44 (d, 1H), 7.24-7.01 (m, 6H), 6.86 (d, 1H), 6.77-6.68 (m, 1H), 6.26-6.17 (m, 1H), 5.89-5.80 (m, 1H), 5.29-5.10 (m, 2H), 4.93-4.80 (m, 1H), 4.61-4.49 (m, 2H), 4.48-4.37 (m, 2H), 4.22-4.06 (m, 3H), 3.97-3.75 (m, 4H), 3.69-3.58 (m, 1H), 3.51-3.40 (m, 4H), 3.30 (s, 3H), 3.02-2.90 (m, 1H), 2.75-2.58 (m, 3H), 2.50-2.30 (m, 6H), 2.18 (s, 3H), 2.01-1.92 (m, 6H). MS (ESI) m/z 1121.1 (M−H)⁻.

### Example 155

### (7R,16R)-19,23-dichloro-10-({2-[4-{[(2R)-1,4-dioxan-2-yl]methoxy}-2-(2,5,8,11-tetraoxadodecan-1-yl)phenyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 155A

### methyl (R)-5-((1,4-dioxan-2-yl)methoxy)-2-bromobenzoate

(S)-(1,4-Dioxan-2-yl)methanol (2500 mg) was dissolved in dichloromethane (10 mL). The mixture was cooled to 0° C. Triethylamine (246 mg) was added. Methanesulfonyl chloride (267 mg) was then added dropwise. The mixture was allowed to warm to ambient temperature. After two hours, saturated aqueous sodium bicarbonate (4 mL) was added. The layers were separated, and the organic portion was washed with brine (5 mL). The aqueous portions were combined and back-extracted with dichloromethane (10 mL). The organic portions were combined and dried over anhydrous sodium sulfate and filtered. The solvent was removed under vacuum. To the residue was added methyl 2-bromo-5-hydroxybenzoate (350 mg) and N,N-dimethylformamide (7 mL). Cesium carbonate (987 mg) was added, and the mixture was heated to 90° C. overnight. The mixture was cooled, and water (20 mL) was added. The solution was extracted with 50% ethyl acetate in heptanes (10 mL) three times. The extracts were combined and washed with water (10 mL) and brine (5 mL). The solution was dried on anhydrous sodium sulfate and filtered. The solvent removed on vacuum to yield the title compound which was utilized without further purification. ¹H NMR (500 MHz, dimethylsulfoxide-d₆) δ ppm 7.60 (d, 1H), 7.29 (d, 1H), 7.08 (dd, 1H), 3.99 (d, 2H), 3.84 (s, 3H), 3.82-3.73 (m, 2H), 3.67-3.57 (m, 2H), 3.51-3.44 (m, 1H), 3.41-3.36 (m, 2H). MS (ESI) m/z 331.2 (M+H)⁺.

### Example 155B

### (R)-(5-((1,4-dioxan-2-yl)methoxy)-2-bromophenyl)methanol

Example 155A (500 mg) was taken up in tetrahydrofuran (4 mL). The solution was cooled in an ice bath to 0° C. Lithium aluminum hydride (2 M in tetrahydrofuran, 0.755 mL) was added dropwise. The solution was stirred for 30 minutes at 0° C. Water (0.5 mL) was added dropwise to quench the reaction, and 2 M aqueous HCl (8 mL) was added to dissolve the metal salts. The solution was allowed to warm to ambient temperature and stirred for 10 minutes. Brine (3 mL) was added, and the solution was extracted with ethyl acetate (20 mL) three times. The extracts were combined, dried on anhydrous sodium sulfate, and filtered. The solvent was removed on vacuum to yield the title compound which was utilized without further purification. ¹H NMR (500 MHz, dimethylsulfoxide-d₆) δ ppm 7.43 (d, 1H), 7.10 (dt, 1H), 6.80 (dd, 1H), 5.45 (t, 1H), 4.45 (d, 2H), 3.95 (d, 2H), 3.87-3.85 (m, 1H), 3.84 (m, 2H), 3.63 (m, 2H), 3.49 (m, 1H), 3.39 (m, 1H).

### Example 155C

### (R)-2-((4-bromo-3-(2,5,8,11-tetraoxadodecyl)phenoxy)methyl)-1,4-dioxane

Example 155B (338 mg) and 2-(2-(2-methoxyethoxy)ethoxy)ethyl methanesulfonate (675 mg) were taken up in 1,4-dioxane (12 mL). Sodium hydride (60° 0, 30.8 mg) was added, and the solution was stirred at ambient temperature for two hours. The reaction was quenched with a few drops of water, and the solution was concentrated on vacuum. The material was taken up in ethyl acetate (20 mL), washed with 0.1 M aqueous sodium hydroxide (5 mL), washed with water (5 mL), washed with brine (5 mL) and dried on anhydrous sodium sulfate. After filtration and concentration, the material was purified by flash column chromatography on silica gel using a gradient of 20-70% ethyl acetate in heptanes. The solvent was removed on vacuum to yield the title compound. ¹H NMR (500 MHz, dimethylsulfoxide-d₆) δ ppm 7.48 (d, 1H), 7.07 (d, 1H), 6.82 (dd, 1H), 4.48 (s, 2H), 4.31 (m, 3H), 3.96 (d, 2H), 3.83-3.72 (m, 2H), 3.68-3.60 (m, 4H), 3.57-3.50 (m, 6H), 3.44-3.40 (m, 4H), 3.24-3.23 (m, 3H). MS (ESI) m/z 466.2 (M+NH₄)⁺.

### Example 155D

### (R)-2-(4-((1,4-dioxan-2-yl)methoxy)-2-(2,5,8,11-tetraoxadodecyl)phenyl)-4,4,5,5-tetramethyl-1,3,2-dioxaborolane

The title compound was prepared by substituting Example 155C for 1-bromo-3-(2-(2-(2-methoxyethoxy)ethoxy)ethoxy)benzene in Example 104B. ¹H NMR (500 MHz, dimethylsulfoxide-d₆) δ ppm 7.59 (d, 1H), 6.99 (d, 1H), 6.84 (dd, 1H), 4.65 (s, 2H), 4.31 (m, 2H), 4.10 (m, 2H), 3.97-3.91 (m, 3H), 3.87-3.72 (m, 4H), 3.68-3.61 (m, 2H), 3.59-3.50 (m, 4H), 3.44-3.38 (m, 4H), 3.24-3.23 (m, 3H), 1.28 (s, 12H). MS (ESI) m/z 514.1 (M+NH₄)⁺.

### Example 155E

### (R)-(2-(4-((1,4-dioxan-2-yl)methoxy)-2-(2,5,8,11-tetraoxadodecyl)phenyl)pyrimidin-4-yl)methanol

The title compound was prepared by substituting Example 155D for tert-butyl 2-(4,4,5,5-tetramethyl-1,3,2-dioxaborolan-2-yl)benzoate in Example 19A. ¹H NMR (500 MHz, dimethylsulfoxide-d₆) δ ppm 8.83 (d, 1H), 7.97 (d, 1H), 7.42 (d, 1H), 7.19 (d, 1H), 6.98 (dd, 1H), 5.65 (t, 1H), 4.90 (s, 2H), 4.60 (d, 2H), 4.03 (d, 2H), 3.93-3.76 (m, 3H), 3.70-3.61 (m, 2H), 3.50 (m, 10H), 3.42 (m, 4H), 3.24-3.23 (m, 3H). MS (ESI) m/z 479.3 (M+H)⁺.

### Example 155F

### (7R,16R)-19,23-dichloro-10-({2-[4-{[(2R)-1,4-dioxan-2-yl]methoxy}-2-(2,5,8,11-tetraoxadodecan-1-yl)phenyl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared by substituting Example 155E for Example 13C in Example 160. ¹H NMR (500 MHz, dimethylsulfoxide-d₆) δ ppm 8.79 (d, 1H), 8.69 (s, 1H), 7.94 (d, 1H), 7.39 (d, 1H), 7.16-7.05 (m, 5H), 6.92 (dd, 1H), 6.84 (d, 1H), 6.72 (dd, 1H), 6.20 (dd, 1H), 5.72 (d, 1H), 5.13 (q, 2H), 4.86 (s, 2H), 4.81 (m, 1H), 4.38 (m, 2H), 3.97 (d, 2H), 3.88 (m, 1H), 3.78 (dd, 1H), 3.71 (dd, 1H), 3.64-3.54 (m, 3H), 3.48-3.44 (m, 6H), 3.43-3.39 (m, 8H), 3.36-3.31 (m, 3H), 3.13 (s, 2H), 2.93 (dd, 1H), 2.71-2.59 (m, 2H), 2.56 (m, 6H), 2.32-2.25 (m, 3H), 1.93 (s, 3H), 1.88 (s, 3H). MS (ESI) m/z 1215.4 (M+H)⁺.

### Example 156

### (7R,16R)-19,23-dichloro-1-(4-fluorophenyl)-10-[(3-{2-[2-(2-methoxyethoxy)ethoxy]ethoxy}phenyl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 156A

### tert-butyl (7R,16R)-19,23-dichloro-1-(4-fluorophenyl)-10-[(3-{2-[2-(2-methoxyethoxy)ethoxy]ethoxy}phenyl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

4 mL vial, equipped with stir bar, was charged with Example 16N (30 mg), (3-(2-[2-(2-methoxyethoxy)ethoxy]ethoxy)phenyl)methanol (30 mg), triphenylphosphine (30 mg) and (E)-N¹,N¹,N²,N²-tetramethyldiazene-1,2-dicarboxamide (TMAD) (20 mg). The mixture was purged for 30 minutes with argon. A mixture of toluene (0.5 mL) and tetrahydrofuran (0.5 mL) was added and the reaction mixture was stirred overnight at ambient temperature. The reaction mixture was then concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-10% methanol in dichloromethane) to afford the title compound. MS (APCI) m/z 1061.60 (M+H)⁺.

### Example 156B

### (7R,16R)-19,23-dichloro-1-(4-fluorophenyl)-10-[(3-{2-[2-(2-methoxyethoxy)ethoxy]ethoxy}phenyl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 156A (34 mg) in dichloromethane (1 mL) was added trifluoroacetic acid (200 μL). The reaction mixture was stirred for 48 hours at ambient temperature.

The reaction mixture was then concentrated in vacuo. The residue was purified by HPLC (Waters X-Bridge C18 19×150 mm 5 μm column, gradient 5-95% acetonitrile+0.1% trifluoroacetic acid in water+0.1% trifluoroacetic acid) to provide the title compound as a trifluoroacetic acid salt. The residue was dissolved in dichloromethane (5 mL) and saturated aqueous NaHCO₃ solution was added. The reaction mixture was stirred for 30 minutes at ambient temperature. The phases were separated with a Horizon DryDisk® and the organic phase was concentrated in vacuo to provide the title compound. ¹H NMR (600 MHz, dimethylsulfoxide-d₆) δ ppm 8.71 (s, 1H), 7.26 (m, 1H), 7.20 (m, 2H), 7.13 (m, 2H), 7.00 (m, 2H), 6.88 (m, 2H), 6.72 (m, 1H), 6.14 (m, 1H), 5.77 (m, 1H), 5.05 (d, 1H), 4.95 (d, 1H), 4.90 (m, 1H), 4.45 (m, 2H), 4.09 (m, 2H), 3.74 (m, 2H), 3.60-3.40 (m, 9H), 3.22 (s, 3H) 2.87 (m, 1H), 2.68 (m, 2H), 2.60-2.25 (m, 10H), 2.17 (s, 3H), 1.97 (s, 3H), 1.93 (s, 3H). MS (APCI) m/z 1005.40 (M+H)⁺.

### Example 157

### (7R,16R)-19,23-dichloro-1-(4-fluorophenyl)-20,22-dimethyl-10-{[2-(4-{[(2S)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 157A

### 4-(4-(((tert-butyldimethylsilyl)oxy)methyl)pyrimidin-2-yl)phenol

A mixture of Example 38A (200.0 mg), 4-(4,4,5,5-tetramethyl-1,3,2-dioxaborolan-2-yl)phenol (179.0 mg) and 1,1′-bis(diphenylphosphino)ferrocene-palladium (II) dichloride dichloromethane complex (50.5 mg) was degassed. Degassed dioxane (3.2 mL) was added followed by degassed sodium carbonate solution (1.1 mL, 2M in water). After heating for 18 hours at 70° C. and cooling to ambient temperature, water was added followed by extraction with ethyl acetate. The combined organic layers were washed with water and dried over magnesium sulfate. After filtration, the solvent was removed in vacuo and the crude product obtained was purified using a Grace Reveleris system (12 g Grace Reveleris column, eluting with 5-50% ethyl acetate in heptane) providing the title compound. MS (APCI) m/z 317.2 (M+H)⁺.

### Example 157B

### (S)-2-((4-(4-(((tert-butyldimethylsilyl)oxy)methyl)pyrimidin-2-yl)phenoxy)methyl)-4-methylmorpholine

A microwave vial was charged with Example 157A (50.0 mg), (S)-4-methyl-2-(hydroxymethyl)morpholine (36.6 mg), triphenylphosphine (83.0 mg) and di-tert-butyl azodicarboxylate (72.8 mg). After degassing, tetrahydrofuran (2.5 mL) was added and the reaction mixture was stirred for 3 hours at ambient temperature followed by heating in the microwave for two hours at 50° C. Triethylamine (17.6 mg) was added and the stirring was continued overnight. After addition of more triphenylphosphine (42.0 mg) and di-tert-butyl azodicarboxylate (37.0 mg), the reaction mixture was stirred for 42 hours at ambient temperature. The solvent was removed in vacuo and the crude product obtained was purified using a Grace Reveleris system (12 g Grace Reveleris column, eluting with 5-75% ethyl acetate/ethanol in heptane) providing the title compound. MS (APCI) m/z 430.4 (M+H)⁺.

### Example 157C

### (S)-(2-(4-((4-methylmorpholin-2-yl)methoxy)phenyl)pyrimidin-4-yl)methanol

Tetra-N-butylammonium fluoride (0.11 mL) was added to an ice-cooled solution of Example 157B (35 mg) in tetrahydrofuran (2 mL). After stirring for 4 hours at 0° C. the reaction mixture was allowed to warm to ambient temperature. Ammonium chloride solution (10% in water) was added and the stirring was continued for 5 minutes. After extraction with ethyl acetate, the combined organic layers were washed with water, dried over magnesium sulfate, filtered and concentrated. The crude product obtained was used without further purification. MS (APCI) m/z 316.2 (M+H)⁺.

### Example 157D

### tert-butyl (7R,16R)-19,23-dichloro-1-(4-fluorophenyl)-20,22-dimethyl-10-{[2-(4-{[(2S)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A mixture of Example 16N (32.0 mg) and Example 157C (17.3 mg) was dried under vacuum for one hour. N,N,N′,N′-Tetramethylazodicarboxamide (20.4 mg) and triphenylphosphine (31.1 mg) were added. After stirring for 15 minutes under argon, a mixture of degassed toluene (0.5 mL) and tetrahydrofuran (0.5 mL) was added and the reaction mixture was stirred for 3 days at ambient temperature. Water was added, followed by extraction with ethyl acetate. The combined organic layers were washed with water, dried over magnesium sulfate, filtered, and concentrated. The crude product was purified by chromatography on silica gel using a Grace Reveleris system (4 g Grace Reveleris column, eluting with 1-20% methanol in dichloromethane) to provide the title compound. MS (APCI) m/z 1106.6 (M+H)⁺.

### Example 157E

### (7R,16R)-19,23-dichloro-1-(4-fluorophenyl)-20,22-dimethyl-10-{[2-(4-{[(2S)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Trifluoroacetic acid (161 mg) was added to a solution of Example 157D (33 mg) in dichloromethane (2 mL) and the reaction mixture was stirred overnight at ambient temperature. Removal of the solvent, followed by purification by HPLC (Waters XBridge C8 19×150 mm 5 μm column, gradient 5-100% acetonitrile+0.2% ammonium hydroxide in water+0.2% ammonium hydroxide) provided the title compound. ¹H NMR (600 MHz, dimethylsulfoxide-d₆) δ ppm 8.82 (d, 1H), 8.74 (s, 1H), 8.33 (m, 2H), 7.44 (d, 1H), 7.24-7.18 (m, 2H), 7.16-7.12 (m, 2H), 7.07 (m, 2H), 6.88 (bd, 1H), 6.75 (bdd, 1H), 6.23 (bm, 1H), 5.80 (bd, 1H), 5.25-5.16 (m, 2H), 4.85 (bm, 1H), 4.44 (bm, 2H), 4.04 (m, 2H), 3.81 (m, 2H), 3.65 (bdd, 1H), 3.55 (td, 1H), 2.98 (bdd, 1H), 2.80 (dt, 1H), 2.71-2.63 (m, 2H), 2.61 (m, 1H), 2.50-2.24 (bm, 8H), 2.20 (s, 3H), 2.15 (s, 3H), 2.02 (m, 1H), 1.99 (s, 3H), 1.95 (s, 3H), 1.90 (t, 1H). MS (APCI) m/z 1050.4 (M+H)⁺.

### Example 158

### (7R,16R)-19,23-dichloro-1-(4-fluorophenyl)-20,22-dimethyl-10-{[2-(4-{[(2R)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 158A

### (R)-4-methyl-2-((4-(4,4,5,5-tetramethyl-1,3,2-dioxaborolan-2-yl)phenoxy)methyl)morpholine

Tetrahydrofuran (6 mL) was added to a degassed mixture of (R)-4-methyl-2-(hydroxymethyl)morpholine (189 mg), 4-(4,4,5,5-tetramethyl-1,3,2-dioxaborolan-2-yl)phenol (200 mg), triphenylphosphine (477 mg) and di-tert-butyl azodicarboxylate (419 mg). The reaction mixture was stirred for 3 days at ambient temperature. Water was added followed by extraction with ethyl acetate. The combined organic layers were washed with water, dried over magnesium sulfate, filtered, and concentrated. The crude product obtained was purified by chromatography on silica gel using a Grace Reveleris system (12 g Buchi Reveleris column, eluting with 5-75% ethyl acetate/ethanol in heptane). The desired fractions were combined and concentrated in vacuo. The precipitate formed was filtered off and washed with heptane. The filtrate was concentrated to dryness providing the title compound which was used without further purification. MS (APCI) m/z 334.3 (M+H)⁺.

### Example 158B

### (R)-2-((4-(4-(((tert-butyldimethylsilyl)oxy)methyl)pyrimidin-2-yl)phenoxy)methyl)-4-methylmorpholine

A degassed solution of Example 158A (221 mg) in dioxane (3 mL) was added to a mixture of Example 38A (130 mg) and 1,1′-bis(diphenylphosphino)ferrocene-palladium (II) dichloride dichloromethane complex (32.8 mg) under argon. After addition of Na₂CO₃ solution (0.75 mL, 2M in water), the reaction mixture was heated for 20 hours at 70° C. and subsequently allowed to cool to ambient temperature. Water was added followed by extraction with ethyl acetate. The combined organic layers were washed with water, dried over magnesium sulfate, filtered, and concentrated. The crude product obtained was purified by chromatography on silica gel using a Grace Reveleris system (12 g Buchi Reveleris column, eluting with 5-75% ethyl acetate/ethanol in heptane) to provide the title compound. MS (APCI) m/z 430.4 (M+H)⁺.

### Example 158C

### (R)-(2-(4-((4-methylmorpholin-2-yl)methoxy)phenyl)pyrimidin-4-yl)methanol

Tetra-N-butylammonium fluoride (0.55 mL) was added to an ice-cooled solution of Example 158B (186.0 mg) in tetrahydrofuran (4 mL). After stirring for 4 hours at 0° C. the reaction mixture was allowed to warm to ambient temperature. Ammonium chloride solution (6 mL, 10% in water) was added and the stirring was continued for 5 minutes. After extraction with ethyl acetate, the combined organic layers were washed with water, dried over magnesium sulfate, filtered, and concentrated. The crude product obtained was purified by chromatography on silica gel using a Grace Reveleris system (4 g Grace Reveleris column, eluting with 1-10% methanol in dichloromethane) to provide the title compound. MS (APCI) m/z 316.2 (M+H)⁺.

### Example 158D

### tert-butyl (7R,16R)-19,23-dichloro-1-(4-fluorophenyl)-20,22-dimethyl-10-{[2-(4-{[(2R)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A mixture of Example 16N (33.0 mg) and Example 158C (20.0 mg) was dried under vacuum for 1 hour. N,N,N′,N′-Tetramethylazodicarboxamide (21.1 mg) and triphenylphosphine (32.1 mg) were added. After stirring for 15 minutes under argon, a mixture of degassed toluene (0.5 mL) and tetrahydrofuran (0.5 mL) was added and the reaction mixture was stirred for 2 days at ambient temperature. Water was added followed by extraction with ethyl acetate. The combined organic layers were washed with water, dried over magnesium sulfate, filtered and concentrated. The crude product was purified by chromatography on silica gel using a Grace Reveleris system (4 g Grace Reveleris column, eluting with 1-20% methanol in dichloromethane) providing the title compound. MS (APCI) m/z 1106.6 (M+H)⁺.

### Example 158E

### (7R,16R)-19,23-dichloro-1-(4-fluorophenyl)-20,22-dimethyl-10-{[2-(4-{[(2R)-4-methylmorpholin-2-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Trifluoroacetic acid (0.122 mL) was added to a solution of Example 158D (37 mg) in dichloromethane (2 mL) and the reaction mixture was stirred overnight at ambient temperature. Additional trifluoroacetic acid (0.061 mL) was added and the stirring was continued for 3 hours. Removal of the solvent, followed by purification by HPLC (first: Waters XSelect CSH C18 30×150 mm 5 μm column, gradient 5% to 100% acetonitrile+0.1% trifluoroacetic acid in water+0.1% trifluoroacetic acid and in a second step: Waters XBridge C8 19×150 mm 5 μm column, gradient 5% to 100% acetonitrile+0.2% ammonium hydroxide in water+0.2% ammonium hydroxide) provided the title compound. ¹H NMR (600 MHz, dimethylsulfoxide-d₆) δ ppm 8.82 (d, 1H), 8.74 (s, 1H), 8.34 (m, 2H), 7.44 (d, 1H), 7.23-7.17 (m, 2H), 7.14 (m, 2H), 7.07 (m, 2H), 6.88 (bd, 1H), 6.75 (bdd, 1H), 6.22 (bs, 1H), 5.81 (bs, 1H), 5.25-5.16 (m, 2H), 4.86 (bm, 1H), 4.44 (bm, 2H), 4.04 (m, 2H), 3.81 (m, 2H), 3.64 (bdd, 1H), 3.55 (td, 1H), 2.98 (bdd, 1H), 2.80 (bdt, 1H), 2.70-2.64 (m, 2H), 2.62 (m, 1H), 2.47-2.24 (bm, 8H), 2.20 (s, 3H), 2.15 (s, 3H), 2.02 (dd, 1H), 1.99 (s, 3H), 1.95 (s, 3H), 1.93-1.86 (m, 1H). MS (APCI) m/z 1050.3 (M+H)⁺.

### Example 159

### (7R,16R)-19,23-dichloro-1-(4-fluorophenyl)-10-[(2-{4-[2-(2-{[(3R,3aR,6R,6aR)-6-methoxyhexahydrofuro[3,2-b]furan-3-yl]oxy}ethoxy)ethoxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 159A

### 4-(4-(((tert-butyldimethylsilyl)oxy)methyl)pyrimidin-2-yl)phenol

A solution of Example 38A (1.5 g), 4-(4,4,5,5-tetramethyl-1,3,2-dioxaborolan-2-yl)phenol (1.3 g) and 1,1′-bis(diphenylphosphino)ferrocene-palladium (II) dichloride dichloromethane complex (0.47 g) in dioxane (24 mL) and 2 M aqueous sodium carbonate (8.6 mL) was purged with nitrogen and heated to 75° C. overnight. The reaction mixture was concentrated, diluted with water and extracted with ethyl acetate three times. The combined organic layers were dried over anhydrous sodium sulfate, filtered and concentrated. The residue was purified by normal phase MPLC on a Teledyne Isco Combiflash® Rf+80 g gold silica gel column eluting, with 0-25% ethyl acetate in heptanes to give the title compound. ¹H NMR (400 MHz, dimethylsulfoxide-d₆) δ ppm 9.93 (br s, 1H), 8.80 (d, 1H), 8.30-8.15 (m, 2H), 7.30 (d, 1H), 6.94-6.80 (m, 2H), 4.78 (s, 2H), 0.93 (s, 9H), 0.12 (s, 6H).

### Example 159B

### 4-(((tert-butyldimethylsilyl)oxy)methyl)-2-(4-(2-(2-(((3R,3aR,6R,6aR)-6-methoxyhexahydrofuro[3,2-b]furan-3-yl)oxy)ethoxy)ethoxy)phenyl)pyrimidine

To a solution of Example 159A (200 mg), Example 133D (235 mg) and triphenylphosphine (250 mg) in tetrahydrofuran (1.9 mL) at ambient temperature was added di-tert-butyl azodicarboxylate (220 mg), and the reaction was heated at 50° C. overnight. The reaction mixture was cooled and concentrated. The residue was purified by normal phase MPLC on a Teledyne Isco Combiflash® Rf+40 g gold silica gel column eluting with 15-70% ethyl acetate in heptanes to give the title compound.

### Example 159C

### (2-(4-(2-(2-(((3R,3aR,6R,6aR)-6-methoxyhexahydrofuro[3,2-b]furan-3-yl)oxy)ethoxy)ethoxy)phenyl)pyrimidin-4-yl)methanol

To a solution of Example 159B (340 mg) in tetrahydrofuran (2.1 mL) and methanol (1 mL) was added cesium fluoride (470 mg), and the reaction was allowed to stir overnight. The reaction was concentrated. The residue was taken up in ethyl acetate, filtered over diatomaceous earth rinsing well with ethyl acetate, and concentrated. The reaction mixture was cooled and concentrated. The residue was purified by normal phase MPLC on a Teledyne Isco Combiflash® Rf+12 g gold silica gel column eluting with 0-4% methanol in dichloromethane to give the title compound. ¹H NMR (400 MHz, dimethylsulfoxide-d₆) δ ppm 8.81 (d, 1H), 8.37-8.29 (m, 2H), 7.40 (d, 1H), 7.11-7.02 (m, 2H), 5.67-5.59 (m, 1H), 4.61 (d, 2H), 4.54-4.46 (m, 2H), 4.22-4.12 (m, 2H), 4.08-3.98 (m, 1H), 3.93-3.81 (m, 3H), 3.81-3.74 (m, 2H), 3.74-3.65 (m, 1H), 3.64-3.52 (m, 3H), 3.47-3.37 (m, 2H), 3.29 (s, 3H).

### Example 159D

### tert-butyl (7R,16R)-19,23-dichloro-1-(4-fluorophenyl)-10-[(2-{4-[2-(2-{[(3R,3aR,6R,6aR)-6-methoxyhexahydrofuro[3,2-b]furan-3-yl]oxy}ethoxy)ethoxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a vial containing Example 16N (40 mg) and Example 159C (64 mg) in toluene (120 μL) and tetrahydrofuran (120 μL) was added triphenylphosphine (39 mg) and N,N,N′,N′-tetramethylazodicarboxamide (26 mg). The reaction mixture was allowed to stir at 50° C. for 5 hours. The reaction was cooled, diluted with ethyl acetate, filtered over diatomaceous earth, and concentrated. The residue was purified by normal phase MPLC on a Teledyne Isco Combiflash® Rf+4 g gold silica gel column, eluting with 0-9% methanol in dichloromethane to give the title compound.

### Example 159E

### (7R,16R)-19,23-dichloro-1-(4-fluorophenyl)-10-[(2-{4-[2-(2-{[(3R,3aR,6R,6aR)-6-methoxyhexahydrofuro[3,2-b]furan-3-yl]oxy}ethoxy)ethoxy]phenyl}pyrimidin-4-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 159D (55 mg) in dichloromethane (220 μL) was added trifluoroacetic acid (220 μL), and the reaction was allowed to stir overnight. The reaction was concentrated under a stream of nitrogen and the residue was taken up in water and acetonitrile. The mixture was purified by RP-HPLC on a Gilson PLC 2020 using a Luna™ column (250×50 mm, 10 mm, 30-80% over 30 minutes with acetonitrile in water containing 10 mM ammonium acetate) to give the title compound. ¹H NMR (400 MHz, dimethylsulfoxide-d₆) δ ppm 8.81 (d, 1H), 8.73 (s, 1H), 8.37-8.28 (m, 2H), 7.44 (d, 1H), 7.23-7.02 (m, 7H), 6.86 (d, 1H), 6.78-6.69 (m, 1H), 6.26-6.18 (m, 1H), 5.87-5.79 (m, 1H), 5.28-5.11 (m, 2H), 4.91-4.81 (m, 1H), 4.54-4.39 (m, 4H), 4.21-4.13 (m, 2H), 4.07-3.97 (m, 1H), 3.92-3.82 (m, 3H), 3.81-3.74 (m, 2H), 3.73-3.53 (m, 6H), 3.47-3.36 (m, 4H), 3.28 (s, 3H). 3.02-2.91 (m, 1H), 2.73-2.58 (m, 2H), 2.50-2.30 (m, 4H), 2.19 (s, 3H), 2.02-1.92 (m, 6H). MS (ESI) m/z 1167.0 (M−H)⁻.

### Example 160

### (7R,16R)-19,23-dichloro-1-(4-fluorophenyl)-10-({2-[(1r,4r)-4-{[2-(2-methoxyethoxy)ethoxy]methyl}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 160A

### 4-(((tert-butyldiphenylsilyl)oxy)methyl)-2-chloropyrimidine

To a flask containing (2-chloropyrimidin-4-yl)methanol (5.00 g) in N,N-dimethylformamide (40 mL) was added tert-butylchlorodiphenylsilane (9.51 g) followed by imidazole (4.71 g). The resulting mixture was stirred at ambient temperature overnight. The mixture was diluted with water (100 mL) and extracted with ethyl acetate (3×150 mL). The organic layer was separated, washed with water and brine, dried over sodium sulfate, filtered and concentrated. The residue was purified by flash chromatography on AnaLogix IntelliFlash²⁸⁰ system (100 g silica gel cartridge, eluting with 0-30% ethyl acetate/hexanes) to give the title compound. MS (ESI) m/z 383.2 (M+H)⁺.

### Example 160B

### ethyl 4-(4-(((tert-butyldiphenylsilyl)oxy)methyl)pyrimidin-2-yl)cyclohex-3-enecarboxylate

A 250 mL flask, equipped with stir bar, was charged with Example 49A (4.00 g), ethyl 4-(4,4,5,5-tetramethyl-1,3,2-dioxaborolan-2-yl)cyclohex-3-enecarboxylate (3.80 g), [1,1′-bis(diphenylphosphino)ferrocene]dichloropalladium (II) (0.764 gl) and potassium phosphate (5.54 g). The flask was capped then evacuated and backfilled with nitrogen twice. Dioxane (55 mL) was added followed by water (13.75 mL) and the stirring mixture was evacuated and backfilled with nitrogen twice again. The mixture was stirred at 80° C. for 16 hours. The mixture was cooled to ambient temperature, poured into a separatory funnel containing water and brine, and extracted three times with ethyl acetate. The organics were combined and concentrated. The residue was purified by flash chromatography on AnaLogix IntelliFlash²⁸⁰ system (100 g silica gel cartridge, eluting with 0-30% ethyl acetate/hexanes) to give the title compound. MS (ESI) m/z 501.2 (M+H)⁺.

### Example 160C

### (4-(4-(((tert-butyldiphenylsilyl)oxy)methyl)pyrimidin-2-yl)cyclohex-3-en-1-yl)methanol

To a solution of Example 49B (2.081 g) in tetrahydrofuran (5 mL) at 0° C. was added lithium diisobutyl-tert-butoxyaluminum hydride (0.25 M in tetrahydrofuran/hexanes, 66.5 mL). The mixture was stirred at 0° C. for 25 minutes. The reaction mixture, at 0° C., was quenched by slow addition of saturated aqueous Rochelle's salt solution (20 ml.) and then stirred at ambient temperature for 15 minutes. The mixture was extracted three times with ethyl acetate and the organies were concentrated. The residue was purified by flash chromatography on an AnaLogix IntelliFlash²⁸⁰ system using a Teledyne Isco RediSep® Rf gold 100 g silica gel column (eluting with 0-100% ethyl acetate/hexanes) to afford the title compound. MS (ESI) m/z 459.4 (M+H)⁺.

### Example 160D

### ((1r,4r)-4-(4-(((tert-butyldiphenylsilyl)oxy)methyl)pyrimidin-2-yl)cyclohexyl)methanol

Example 160C (2.095 g) and tetrahydrofuran (14.5 mL) were added to Ra-Ni 2800 water slurry (2.0 g) in a 25 mL Hast C reactor, and the mixture was stirred at 50 psi hydrogen for one hour. The reaction mixture was filtered and concentrated. The residue was purified by flash chromatography on an AnaLogix IntelliFlash²⁸⁰ system using a Teledyne Isco RediSep® Rf gold 100 g silica gel column (eluting with 20-100% ethyl acetate/hexanes) to afford the title compound. ¹H NMR (400 MHz, dimethylsulfoxide-d₆) δ ppm 8.75 (d, 1H), 7.64 (dt, 4H), 7.43 (dddd, 7H), 4.72 (s, 2H), 4.37 (s, 1H), 3.28-3.15 (m, 2H), 2.65 (tt, 1H), 1.96-1.77 (m, 4H), 1.58-1.31 (m, 3H), 1.05 (s, 9H), 1.04-0.93 (m, 2H). MS (ESI) m/z 461.3 (M+H)⁺.

### Example 160E

### 4-(((tert-butyldiphenylsilyl)oxy)methyl)-2-((1r,4r)-4-((2-(2-methoxyethoxy)ethoxy)methyl)cyclohexyl)pyrimidine

To a stirring solution of Example 160D (200 mg) in tetrahydrofuran (4 mL) was slowly added sodium hydride (52.1 mg) and the mixture was stirred for 25 minutes. 1-Bromo-2-(2-methoxyethoxy) ethane (265 mg) was added and the mixture was stirred at 45° C. for 2 days. One drop of saturated aqueous ammonium chloride solution was added. The mixture was filtered to remove the material and the material was washed with dichloromethane. The organics were concentrated. The residue was purified by flash chromatography on an AnaLogix IntelliFlash²⁸⁰ system using a Teledyne Isco RediSep® Rf gold 24 g silica gel column (eluting with 10-60% ethyl acetate/hexanes over 30 minutes) to afford the title compound. MS (ESI) m/z 563.3 (M+H)⁺.

### Example 160F

### (2-((1r,4r)-4-((2-(2-methoxyethoxy)ethoxy)methyl)cyclohexyl)pyrimidin-4-yl)methanol

To a stirring solution of Example 160E (150 mg) in tetrahydrofuran (1 mL) was slowly added tetrabutylammonium fluoride (1.0 M in tetrahydrofuran, 0.533 mL). The mixture was stirred for one hour. The reaction mixture was concentrated and purified by flash chromatography on an AnaLogix IntelliFlash²⁸⁰ system using a Teledyne Isco RediSep® Rf gold 24 g silica gel column (solvent A=3:1 ethyl acetate/ethanol, solvent B=heptane, eluting with 10-90% A to B) to afford the title compound. MS (ESI) m/z 325.3 (M+H)⁺.

### Example 160G

### tert-butyl (7R,16R)-19,23-dichloro-1-(4-fluorophenyl)-10-({2-[(1r,4r)-4-{[2-(2-methoxyethoxy)ethoxy]methyl}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (90 mg), Example 160F (72.1 mg) and triphenylphosphine (61.2 mg). The vial was capped with septa then evacuated and backfilled with nitrogen. Toluene (1.8 mL) was added and the mixture was cooled with an ice bath. Di-tert-butyl azodicarboxylate (51.2 mg) was added in one solid portion, and the vial was capped with septa, evacuated and backfilled with nitrogen twice. The mixture was stirred at 0° C. for 10 minutes. The cooling bath was removed and the mixture was allowed to stir for one day. The mixture was concentrated and purified by flash chromatography on an AnaLogix IntelliFlash²⁸⁰ system using a Teledyne Isco RediSep® Rf gold 12 g silica gel column (eluting with 4-16% methanol/dichloromethane over 35 minutes) afforded the title compound. MS (ESI) m/z 1117.7 (M+H)⁺.

### Example 160H

### (7R,16R)-19,23-dichloro-1-(4-fluorophenyl)-10-({2-[(1r,4r)-4-{[2-(2-methoxyethoxy)ethoxy]methyl}cyclohexyl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 160G (108 mg) in dichloromethanez (1 mL) was added trifluoroacetic acid (1 mL). The mixture was stirred for 3 hours. The mixture was concentrated in vacuo and purified by reverse phase prep LC using Phenomenex® Luna™ C-18 250×50 mm column, 70 mL/minutes flow, 10-95% acetonitrile in 10 mM ammonium acetate in water over 35 minutes. The title compound was obtained after lyophilization. ¹H NMR (400 MHz, dimethylsulfoxide-d₆) δ ppm 8.78-8.60 (m, 2H), 7.41 (d, 1H), 7.26-7.04 (m, 4H), 6.83 (d, 1H), 6.73 (dd, 1H), 6.21 (dd, 1H), 5.81 (d, 1H), 5.09 (q, 2H), 4.87 (p, 1H), 4.43 (d, 2H), 3.67-3.26 (m, 13H), 3.24 (s, 3H), 2.99-2.60 (m, 6H), 2.59-2.34 (m, 4H), 2.22 (s, 3H), 1.97 (s, 6H), 1.95-1.79 (m, 4H), 1.56 (qd, 3H), 1.17-0.97 (m, 2H). MS (ESI) m/z 1061.2 (M+H)⁺.

### Example 161

### (7R,16R)-19,23-dichloro-1-(4-fluorophenyl)-10-{[2-(4-{[(3S,3aR,6R,6aR)-6-hydroxyhexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 161A

### (3R,3aR,6R,6aS)-6-((tert-butyldiphenylsilyl)oxy) hexahydrofuro[3,2-b]furan-3-ol

To a solution of (3R,3aR,6R,6aR)-hexahydrofuro[3,2-b]furan-3,6-diol (3 g) and imidazole (2.8 g) in dichloromethane (72 mL) at 0° C. was added tert-butylchlorodiphenylsilane (5.8 mL), and the reaction was allowed to stir overnight. The reaction was diluted with saturated aqueous ammonium chloride and extracted with dichloromethane three times. The combined organic layers were dried over anhydrous sodium sulfate, filtered and concentrated. The residue was purified by normal phase MPLC on a Teledyne Isco Combiflash® Rf+80 g gold silica gel column eluting with 0-45% ethyl acetate in heptanes to give the title compound. ¹H NMR (500 MHz, CDCl₃) δ ppm 7.78-7.72 (m, 2H), 7.71-7.65 (m, 2H), 7.48-7.36 (m, 6H), 4.38-4.32 (m, 1H), 4.28-4.19 (m, 3H), 4.02 (dd, 1H), 3.80-3.71 (m, 2H), 3.69-3.62 (m, 1H), 2.93 (d, 1H), 1.10 (s, 9H).

### Example 161B

### (2-chloropyrimidin-4-yl)methyl acetate

To a solution of (2-chloropyrimidin-4-yl)methanol (1.6 g) in dichloromethane (18 mL) at 0° C. was added pyridine (3.5 mL) followed by acetic anhydride (2 mL), and the reaction was allowed to warm to ambient temperature. After 3.5 hours, the reaction was cooled to 0° C., diluted with dichloromethane, quenched with saturated aqueous sodium bicarbonate, and extracted with dichloromethane three times. The combined organics were washed with water and brine, dried over anhydrous sodium sulfate, filtered and concentrated. The residue was purified by normal phase MPLC on a Teledyne Isco Combiflash® Rf+80 g gold silica gel column eluting with 5-45% ethyl acetate in heptanes to give the title compound. ¹H NMR (400 MHz, dimethylsulfoxide-d₆) δ ppm 8.77 (d, 1H), 7.57 (d, 1H), 5.17 (s, 2H), 2.16 (s, 3H).

### Example 161C

### (2-(4-hydroxyphenyl)pyrimidin-4-yl)methyl acetate

To a solution of 4-(4,4,5,5-tetramethyl-1,3,2-dioxaborolan-2-yl)phenol (470 mg) and Example 161B (400 mg) in tetrahydrofuran (9.1 mL) and saturated aqueous sodium bicarbonate solution (5.2 mL) was added tetrakis(triphenylphosphine)palladium (0) (250 mg), and the reaction was purged with nitrogen and heated to 75° C. overnight. The reaction was cooled, diluted with ethyl acetate and water, and the aqueous layer was extracted with ethyl acetate three times. The combined organic layers were dried over anhydrous sodium sulfate, filtered and concentrated. The residue was purified by normal phase MPLC on a Teledyne Isco Combiflash® Rf+80 g gold silica gel column eluting with 0-50% ethyl acetate in heptanes to give the title compound. ¹H NMR (400 MHz, dimethylsulfoxide-d₆) δ ppm 9.98 (br s, 1H), 8.79 (d, 1H), 8.30-8.18 (m, 2H), 7.27 (d, 1H), 6.95-6.81 (m, 2H), 5.19 (s, 2H), 2.19 (s, 3H).

### Example 161D

### (2-(4-(((3S,3aR,6R,6aS)-6-((tert-butyldiphenylsilyl)oxy) hexahydrofuro[3,2-b]furan-3-yl)oxy)phenyl)pyrimidin-4-yl)methyl acetate

To a solution of Example 161C (100 mg), Example 161A (240 mg) and triphenylphosphine (160 mg) in tetrahydrofuran (1.2 mL) at ambient temperature was added di-tert-butyl azodicarboxylate (140 mg), and the reaction was allowed to stir at 50° C. overnight. The reaction was cooled and concentrated. The residue was purified by normal phase MPLC on a Teledyne Isco Combiflash® Rf+24 g gold silica gel column eluting with 0-15% ethyl acetate in dichloromethane. The desired fractions were combined, concentrated and purified by normal phase MPLC on a Teledyne Isco Combiflash® Rf+40 g gold silica gel column eluting with 0-30% ethyl acetate in heptanes to give the title compound. ¹H NMR (500 MHz, dimethylsulfoxide-d₆) δ ppm 8.83 (d, 1H), 8.37-8.29 (m, 2H), 7.73-7.67 (m, 2H), 7.66-7.60 (m, 2H), 7.52-7.41 (m, 6H), 7.33 (d, 1H), 7.12-7.04 (m, 2H), 5.21 (s, 2H), 4.99-4.94 (m, 1H), 4.50-4.43 (m, 2H), 4.30-4.22 (m, 1H), 4.19 (dd, 1H), 4.09-3.99 (m, 1H), 3.65 (dd, 1H), 3.55 (dd, 1H), 2.19 (s, 3H), 1.03 (s, 9H).

### Example 161E

### (2-(4-(((3S,3aR,6R,6aS)-6-((tert-butyldiphenylsilyl)oxy) hexahydrofuro[3,2-b]furan-3-yl)oxy)phenyl)pyrimidin-4-yl)methanol

To a solution of Example 161D (130 mg) in methanol (590 μL) and tetrahydrofuran (150 μL) was added potassium carbonate (120 mg), and the reaction was allowed to stir at ambient temperature. After 2 hours, the reaction was filtered, washed with ethyl acetate, diluted with water and extracted with ethyl acetate three times. The combined organic layers were dried over anhydrous sodium sulfate, filtered and concentrated. The residue was purified by normal phase MPLC on a Teledyne Isco Combiflash® Rf+12 g gold silica gel column eluting with 5-55% ethyl acetate in heptanes to give the title compound. ¹H NMR (400 MHz, dimethylsulfoxide-d₆) δ ppm 8.81 (d, 1H), 8.39-8.27 (m, 2H), 7.74-7.67 (m, 2H), 7.66-7.59 (m, 2H), 7.54-7.37 (m, 7H), 7.12-7.01 (m, 2H), 5.68-5.58 (m, 1H), 5.02-4.90 (m, 1H), 4.67-4.56 (m, 2H), 4.51-4.41 (m, 2H), 4.32-4.15 (m, 2H), 4.10-4.00 (m, 1H), 3.70-3.60 (m, 1H), 3.59-3.50 (m, 1H), 1.03 (s, 9H).

### Example 161F

### tert-butyl (7R,16R)-19,23-dichloro-1-(4-fluorophenyl)-10-{[2-(4-{[(3S,3aR,6R,6aR)-6-((tert-butyldiphenylsilyl)oxy) hexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A vial containing Example 16N (55 mg) and Example 161E (77 mg) was azeotroped with toluene and tetrahydrofuran three times. The mixture was taken up in toluene (170 μL) and tetrahydrofuran (170 μL), triphenylphosphine (53 mg) and N,N,N′,N′-tetramethylazodicarboxamide (35 mg) were added. The reaction mixture was allowed to stir at 50° C. for 4 hours. The reaction was cooled, diluted with ethyl acetate, filtered over diatomaceous earth and concentrated. The residue was purified by normal phase MPLC on a Teledyne Isco Combiflash® Rf+12 g gold silica gel column eluting with 0-6% methanol in dichloromethane to give the title compound.

### Example 161G

### tert-butyl (7R,16R)-19,23-dichloro-1-(4-fluorophenyl)-10-{[2-(4-{[(3S,3aR,6R,6aR)-6-hydroxyhexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 161G (92 mg) in tetrahydrofuran (340 μL) was added tetrabutylammonium fluoride (100 μL, 1 M in tetrahydrofuran), and the reaction was allowed to stir for 45 minutes. The reaction was diluted with water and methanol. The mixture was reduced under vacuum and extracted with ethyl acetate three times. The combined organic layers were dried over anhydrous sodium sulfate, filtered and concentrated. The residue was purified by normal phase MPLC on a Teledyne Isco Combiflash® Rf+12 g gold silica gel column eluting with 2-8% methanol in dichloromethane to give the title compound.

### Example 161H

### (7R,16R)-19,23-dichloro-1-(4-fluorophenyl)-10-{[2-(4-{[(3S,3aR,6R,6aR)-6-hydroxyhexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 161G (33 mg) in dichloromethane (200 μL) was added trifluoroacetic acid (200 μL), and the reaction was allowed to stir for 5 hours. The reaction was concentrated under a stream of nitrogen and taken up in water and acetonitrile. The mixture was purified by RP-HPLC on a Gilson PLC 2020 using a Luna™ column (250×50 mm, 10 mm) (30-80% over 30 minutes with acetonitrile in water containing 10 mM ammonium acetate) to give the title compound. ¹H NMR (400 MHz, dimethylsulfoxide-d₆) δ ppm 8.82 (d, 1H), 8.73 (s, 1H), 8.39-8.30 (m, 2H), 7.45 (d, 1H), 7.23-7.04 (m, 7H), 6.87 (d, 1H), 6.78-6.70 (m, 1H), 6.27-6.18 (m, 1H), 5.87-5.78 (m, 1H), 5.29-5.10 (m, 2H), 4.94-4.80 (m, 2H), 4.56-4.37 (m, 4H), 4.20-4.11 (m, 2H), 4.08 (dd, 1H), 3.97 (d, 1H), 3.78 (dd, 1H), 3.69-3.60 (m, 1H), 3.49-3.40 (m, 1H), 3.01-2.92 (m, 1H), 2.74-2.59 (m, 2H), 2.49-2.30 (m, 6H), 2.19 (s, 3H), 2.01-1.92 (m, 6H). MS (ESI) m/z 1065.3 (M−H)⁻.

### Example 162

### (7R,16R)-19,23-dichloro-1-(4-fluorophenyl)-10-[(5-{2-[2-(2-methoxyethoxy)ethoxy]ethoxy}pyridin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 162A

### tert-butyl (7R,16R)-19,23-dichloro-1-(4-fluorophenyl)-10-[(5-{2-[2-(2-methoxyethoxy)ethoxy]ethoxy}pyridin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (30 mg), (5-(2-[2-(2-methoxyethoxy)ethoxy]ethoxy)pyridine-2-yl)methanol (30 mg), triphenylphosphine (30 mg) and (E)-N¹,N¹,N²,N²-tetramethyldiazene-1,2-dicarboxamide (TMAD) (20 mg). The mixture was purged for 30 minutes with argon. A mixture of toluene (0.5 mL) and tetrahydrofuran (0.5 mL) was added and the reaction mixture was stirred overnight at ambient temperature. The reaction mixture was then concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting 0-50% methanol in dichloromethane) to afford the title compound. MS (APCI) m/z 1062.6 (M+H)⁺.

### Example 162B

### (7R,16R)-19,23-dichloro-1-(4-fluorophenyl)-10-[(5-{2-[2-(2-methoxyethoxy)ethoxy]ethoxy}pyridin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 162A (42 mg) in dichloromethane (1 mL) was added trifluoroacetic acid (250 μL). The reaction mixture was stirred for 5 days at ambient temperature. The reaction mixture was then concentrated in vacuo. The residue was purified by HPLC Purification (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.74 (s, 1H), 8.26 (d, 1H), 7.44 (d, 1H), 7.40 (d, 1H), 7.20 (m, 2H), 7.14 (m, 2H), 6.87 (d, 1H), 6.74 (m, 1H), 6.19 (m, 1H), 5.76 (m, 1H), 5.05 (m, 1H), 5.00 (m, 1H), 4.86 (m, 1H), 4.44 (m, 2H), 4.17 (m, 2H), 3.75 (m, 2H), 3.60-3.50 (m, 7H), 3.42 (m, 2H), 3.22 (s, 3H), 2.88 (m, 1H), 2.33 (m, 2H), 2.55-2.25 (m, 8H), 2.20 (s, 3H), 1.99 (s, 3H), 1.96 (s, 3H). MS (APCI) m/z 1006.3 (M+H)⁺

### Example 163

### (7R,16R)-19,23-dichloro-1-(4-fluorophenyl)-20,22-dimethyl-10-{[2-(4-{[(3R)-4-methylmorpholin-3-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 163A

### (R)-3-((4-(4-(((tert-butyldimethylsilyl)oxy)methyl)pyrimidin-2-yl)phenoxy)methyl)-4-methylmorpholine

A microwave vial was charged with Example 157A (100.0 mg), (S)-4-methyl-3-(hydroxymethyl)morpholine (83 mg), triphenylphosphine (166.0 mg) and di-tert-butyl azodicarboxylate (146.0 mg). After degassing, tetrahydrofuran (3 mL) was added and the reaction mixture was stirred for 3 days at ambient temperature. Water was added followed by extraction with ethyl acetate. The combined organic layers were washed with water, dried over magnesium sulfate, and filtered. The solvent was removed in vacuo and the crude product obtained was purified using a Grace Reveleris system (12 g Grace Reveleris column, eluting with 2-60% ethyl acetate/ethanol in heptane) to provide the title compound which was used in the next step without further purification. MS (APCI) m/z 430.2 (M+H)⁺.

### Example 163B

### (R)-(2-(4-((4-methylmorpholin-3-yl)methoxy)phenyl)pyrimidin-4-yl)methanol

Tetra-N-butylammonium fluoride (0.277 mL) was added to an ice-cooled solution of Example 163A (118.8 mg, 50% pure) in tetrahydrofuran (2 mL). The reaction mixture was slowly warmed to ambient temperature and stirred overnight. Ammonium chloride solution (2 mL, 10% in water) was added and the stirring was continued for 5 minutes. After extraction with ethyl acetate, the combined organic layers were washed with brine, dried over magnesium sulfate, and filtered. The crude material obtained was purified by SFC (Luna™ HILIC 150×30 mm 5 μm column, eluting with 5-15% methanol+0.2% ammonium hydroxide (25% in water) in liquid CO₂) providing the title compound. MS (APCI) m/z 316.2 (M+H)⁺.

### Example 163C

### tert-butyl (7R,16R)-19,23-dichloro-1-(4-fluorophenyl)-20,22-dimethyl-10-{[2-(4-{[(3R)-4-methylmorpholin-3-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A mixture of Example 16N (18.0 mg) and Example 163B (8.0 mg) was dried under vacuum for 1 hour. N,N,N′,N′-Tetramethylazodicarboxamide (15.3 mg) and triphenylphosphine (23.3 mg) were added. After stirring for 15 minutes under argon, a mixture of degassed toluene (0.5 mL) and tetrahydrofuran (0.5 mL) was added and the reaction mixture was stirred for 3 days at ambient temperature. Water was added followed by extraction with ethyl acetate. The combined organic layers were washed with water, dried over magnesium sulfate, filtered, and concentrated. The crude product was purified by chromatography on silica gel using a Grace Reveleris system (4 g Grace Reveleris column, eluting with 1-15% methanol in dichloromethane) to provide the title compound. MS (APCI) m/z 1106.6 (M+H)⁺.

### Example 163D

### (7R,16R)-19,23-dichloro-1-(4-fluorophenyl)-20,22-dimethyl-10-{[2-(4-{[(3R)-4-methylmorpholin-3-yl]methoxy}phenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Trifluoroacetic acid (0.067 mL) was added to a solution of Example 163C (16 mg) in dichloromethane (2 mL) and the reaction mixture was stirred overnight at ambient temperature. Additional trifluoroacetic acid (0.05 mL) was added and the stirring was continued for 24 hours. Removal of the solvent, followed by purification by HPLC (Waters XBridge C8 19×150 mm 5 μm column, gradient 5% to 100% acetonitrile+0.2% ammonium hydroxide in water+0.2% ammonium hydroxide) provided the title compound. ¹H NMR (600 MHz, dimethylsulfoxide-d₆) δ 8.82 (d, 1H), 8.74 (s, 1H), 8.34 (m, 2H), 7.44 (d, 1H), 7.20 (m, 2H), 7.14 (m, 2H), 7.08 (m, 2H), 6.88 (d, 1H), 6.75 (bdd, 1H), 6.24 (bs, 1H), 5.80 (bs, 1H), 5.28-5.15 (m, 2H), 4.85 (bm, 1H), 4.44 (m, 2H), 4.19 (dd, 1H), 3.97 (dd, 1H), 3.87 (dd, 1H), 3.70 (dt, 1H), 3.65 (bdd, 1H), 3.52 (m, 1H), 3.37 (m, 1H), 2.98 (m, 1H), 2.72-2.64 (m, 3H), 2.48-2.32 (bm, 9H), 2.30 (s, 3H), 2.24 (ddd, 1H), 2.15 (s, 3H), 1.99 (s, 3H), 1.95 (s, 3H). MS (APCI) m/z 1050.3 (M+H)⁺.

### Example 164

### (7R,16R)-19,23-dichloro-1-(4-fluorophenyl)-20,22-dimethyl-16-[(4-methylpiperazin-1-yl)methyl]-10-{[2-(4-{2-[(3aR,6aS)-tetrahydro-1H-furo[3,4-c]pyrrol-5 (3H)-yl]ethoxy}phenyl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 164A (3aR,6aS)-5-(2-(4-(4-(((tert-butyldimethylsilyl)oxy)methyl)pyrimidin-2-yl)phenoxy)ethyl) hexahydro-1H-furo[3,4-c]pyrrole

A 4 mL vial, equipped with stir bar, charged with Example 157A (100 mg), 2-((3aR,6aS)-tetrahydro-1H-furo[3,4-c]pyrrol-5 (3H)-yl)ethanol (74.5 mg) and triphenylphosphine (124 mg), was purged for 30 minutes with argon. Tetrahydrofuran (958 μL) was added and subsequently di-tert-butyl azaodicarboxylate (DBAD) (109 mg) was added and the reaction mixture was stirred overnight at ambient temperature and for 24 hours at 30° C. To the reaction mixture was added di-tert-butyl azaodicarboxylate (DBAD) (72.8 mg) and triphenylphosphine (83 mg) and the reaction mixture was stirred 22 hours at 30° C. The reaction mixture was concentrated in vacuo. The residue was purified by normal phase MPLC on a Teledyne-Isco-Combiflash® system (12 g Agela Si Spherical (20-35 μm), eluting first with 0-50% ethyl acetate in n-heptane and then 10% ethanol was added to the ethyl acetate eluent) to afford the title compound. MS (APCI) m/z 456.3 (M+H)⁺.

### Example 164B

### (2-(4-(2-((3aR,6aS)-tetrahydro-1H-furo[3,4-c]pyrrol-5 (3H)-yl)ethoxy)phenyl)pyrimidin-4-yl)methanol

To a solution of Example 164A (99 mg) in tetrahydrofuran (724 μL) was added cesium fluoride (83 mg). Subsequently methanol (362 μL) was added and the reaction mixture was stirred for 17 hours at ambient temperature. The reaction mixture was concentrated in vacuo and the residue was purified by normal phase MPLC on a Teledyne-Isco-Combiflash® system (eluting 0-10% methanol in dichloromethane) to afford the title compound. MS (APCI) m/z 342.3 (M+H)⁺.

### Example 164C

### tert-butyl (7R,16R)-19,23-dichloro-1-(4-fluorophenyl)-20,22-dimethyl-16-[(4-methylpiperazin-1-yl)methyl]-10-{[2-(4-{2-[(3aR,6aS)-tetrahydro-1H-furo[3,4-c]pyrrol-5 (3H)-yl]ethoxy}phenyl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (35 mg), Example 164B (26.6 mg), triphenylphosphine (45.3 mg) and (E)-N¹,N¹,N²,N²-tetramethyldiazene-1,2-dicarboxamide (TMAD) (29.8 mg), and was purged for 30 minutes with argon. A mixture of toluene (0.5 mL) and tetrahydrofuran (0.5 mL) was added and the reaction mixture was stirred for 18 hours at ambient temperature. Dichloromethane was added to the reaction mixture and the organic phase was extracted twice with water and brine and subsequently dried via DryDisk®. The organic phase was concentrated in vacuo. The residue was purified by normal phase MPLC on a Teledyne-Isco-Combiflash® system (eluting 0-20% methanol in dichloromethane) to afford the title compound. MS (APCI) m/z 1132.40 (M+H)⁺.

### Example 164D

### (7R,16R)-19,23-dichloro-1-(4-fluorophenyl)-20,22-dimethyl-16-[(4-methylpiperazin-1-yl)methyl]-10-{[2-(4-{2-[(3aR,6aS)-tetrahydro-1H-furo[3,4-c]pyrrol-5 (3H)-yl]ethoxy}phenyl)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 164C (43 mg) in dichloromethane (406 μL) was added trifluoroacetic acid (234 μL). The reaction mixture was stirred for 4 hours at ambient temperature. The reaction mixture was then concentrated in vacuo. The residue was purified by HPLC purification (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.82 (d, 1H), 8.73 (s, 1H), 8.34 (m, 2H), 7.44 (d, 1H), 7.20 (m, 2H), 7.14 (m, 2H), 7.07 (m, 2H), 6.88 (d, 1H), 6.74 (d, 1H), 6.21 (m, 1H), 5.81 (m, 1H), 5.21 (d, 1H), 5.15 (d, 1H), 4.87 (m, 1H), 4.44 (m, 2H), 4.14 (m, 2H), 3.70 (m, 2H), 3.63 (m, 1H), 3.40 (m, 2H), 2.98 (m, 1H), 2.78 (m, 2H), 2.70-2.60 (m, 6H), 2.55-2.25 (m, 10H), 2.14 (s, 3H), 1.99 (s, 3H), 1.97 (s, 3H). MS (APCI) m/z 1076.30 (M+H)⁺.

### Example 165

### (7R,16R)-19,23-dichloro-1-(4-fluorophenyl)-20,22-dimethyl-16-[(4-methylpiperazin-1-yl)methyl]-10-[(2-{4-[2-(2-oxa-6-azaspiro[3.3]heptan-6-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 165A

### 6-(2-(4-(4-(((tert-butyldimethylsilyl)oxy)methyl)pyrimidin-2-yl)phenoxy)ethyl)-2-oxa-6-azaspiro[3.3]heptane

A 4 mL vial, equipped with stir bar, was charged with Example 157A (100 mg), 2-(2-oxa-6-azaspiro[3.3]heptan-6-yl)ethanol (67.9 mg) and triphenylphosphine (124 mg) and was purged for 30 minutes with argon. Tetrahydrofuran (958 μL) was added and subsequently di-tert-butyl azaodicarboxylate (DBAD) (109 mg) was added and the reaction mixture was stirred for 1 hour at 30° C. The reaction mixture was concentrated in vacuo. The residue was purified by normal phase MPLC on a Teledyne-Isco-Combiflash® system (24 g Flashpur Alumina neutral (60 μm), eluting with 0-80% ethyl acetate in heptane) to afford the title compound. MS (APCI) m/z 442.30 (M+H)⁺.

### Example 165B

### (2-(4-(2-(2-oxa-6-azaspiro[3.3]heptan-6-yl)ethoxy)phenyl)pyrimidin-4-yl)methanol

To a solution of Example 165A (122 mg) in tetrahydrofuran (921 μL) was added CsF (105 mg). Subsequently methanol (460 μL) was added and the reaction mixture was stirred for 22 hours at ambient temperature. The reaction mixture was concentrated in vacuo and the residue was purified by normal phase MPLC on a Teledyne-Isco-Combiflash® system (Flash Pure 24 g ALOX neutral; eluting with 0-5% methanol in dichloromethane) to afford the title compound. MS (APCI) m/z 328.20 (M+H)⁺.

### Example 165C

### tert-butyl (7R,16R)-19,23-dichloro-1-(4-fluorophenyl)-20,22-dimethyl-16-[(4-methylpiperazin-1-yl)methyl]-10-[(2-{4-[2-(2-oxa-6-azaspiro[3.3]heptan-6-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (35 mg), Example 165B (20.9 mg), triphenylphosphine (45.3 mg) and (E)-N¹,N¹,N², N²-tetramethyldiazene-1,2-dicarboxamide (TMAD) (29.8 mg) and was purged for 30 minutes with argon. A mixture of toluene (0.5 mL) and tetrahydrofuran (0.5 mL) was added and the reaction mixture was stirred for 18 hours at ambient temperature and 23 hours at 30° C. To the reaction mixture was added triphenylphosphine (22.6 mg) and di-tert-butyl azodicarboxylate (14.9 mg) and stirring was continued for 3 days at 30° C. The reaction mixture was concentrated in vacuo. The residue was purified by normal phase MPLC on a Teledyne-Isco-Combiflash® system (eluting 0-20% methanol in dichloromethane) to afford the title compound. MS (APCI) m/z 1118.3 (M+H)⁺.

### Example 165D

### (7R,16R)-19,23-dichloro-1-(4-fluorophenyl)-20,22-dimethyl-16-[(4-methylpiperazin-1-yl)methyl]-10-[(2-{4-[2-(2-oxa-6-azaspiro[3.3]heptan-6-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 165C (34 mg) in dichloromethane (234 μL) was added trifluoroacetic acid (234 μL). The reaction mixture was stirred for 2 hours and 20 minutes at ambient temperature. The reaction mixture was then concentrated in vacuo. The residue was purified by HPLC Purification (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.81 (d, 1H), 8.70 (s, 1H), 8.33 (m, 2H), 7.47 (m, 1H), 7.19 (m, 2H), 7.13 (m, 2H), 7.03 (m, 2H), 6.85 (m, 1H), 6.71 (m, 1H), 6.14 (m, 1H), 5.88 (m, 1H), 5.20 (d, 1H), 5.15 (d, 1H), 4.90 (m, 1H), 4.60 (s, 4H), 4.43 (m, 2H), 3.99 (m, 2H), 3.37 (m, 4H), 2.97 (m, 1H), 2.75-2.55 (m, 4H), 2.50-2.25 (m, 9H), 2.14 (s, 3H), 2.00 (s, 3H), 1.97 (s, 3H). MS (APCI) m/z 1062.3 (M+H)⁺.

### Example 166

### (7R,16R)-19,23-dichloro-1-(4-fluorophenyl)-20,22-dimethyl-16-[(4-methylpiperazin-1-yl)methyl]-10-[(2-{4-[2-(tetrahydro-1H-furo[3,4-c]pyrrol-5 (3H)-yl)ethyl]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 166A

### 2-(4-(4-(((tert-butyldimethylsilyl)oxy)methyl)pyrimidin-2-yl)phenyl) ethan-1-ol

Example 94A (200 mg), 2-[4-(tetramethyl-1,3,2-dioxaborolan-2-yl)phenyl]ethan-1-ol (202 mg), and 1,1′-bis(diphenylphosphino)ferrocene-palladium (II) dichloride dichloromethane complex (52 mg) were combined under argon. 1,4-Dioxane (4.0 mL, degassed with argon) and aqueous sodium carbonate solution (2M, 1.16 mL, degassed with argon) were added. The reaction mixture was stirred overnight at 70° C. The reaction mixture was concentrated and the residue was partitioned between water and ethyl acetate. The aqueous layer was extracted with ethyl acetate. The combined organic layers were washed with water and brine, dried over magnesium sulfate, filtrated, and concentrated. Purification was performed on a silica gel column (12 g, 0-10% methanol in dichloromethane). The desired fractions were combined and the solvents were removed under reduced pressure to provide the title compound. MS (APCI) m/z 345.3 (M+H)⁺.

### Example 166B

### 4-(4-(((tert-butyldimethylsilyl)oxy)methyl)pyrimidin-2-yl) phenethyl methanesulfonate

Example 166A (252 mg) was dissolved in dichloromethane under nitrogen atmosphere and cooled with iced water. Triethylamine (310 μL) and methanesulfonyl chloride (70 μL) were added. The reaction mixture was stirred at 0° C. for 1 hour. The reaction mixture was diluted with brine. The aqueous layer was extracted with dichloromethane two times. The combined organic extracts were dried over magnesium sulfate, filtrated and concentrated to yield the crude title compound, which was used in the next step without further purification. MS (ESI) m/z 423.2 (M+H)⁺.

### Example 166C

### 5-(4-(4-(((tert-butyldimethylsilyl)oxy)methyl)pyrimidin-2-yl) phenethyl) hexahydro-1H-furo[3,4-c]pyrrole

Example 166B (220 mg), hexahydro-1H-furo[3,4-c]pyrrole (80 mg), and sodium carbonate (160 mg) were combined with acetonitrile (5.0 mL). The reaction mixture was stirred overnight at 70° C. The reaction mixture was partitioned between water and ethyl acetate. The organic layer was washed with brine and concentrated. Purification was performed on a silica gel column (4 g, 5-10% methanol in dichloromethane). The desired fractions were combined and the solvents were removed under reduced pressure to provide the title compound. MS (ESI) m/z 440.3 (M+H)⁺.

### Example 166D

### (2-(4-(2-(tetrahydro-1H-furo[3,4-c]pyrrol-5 (3H)-yl)ethyl)phenyl)pyrimidin-4-yl)methanol

Example 166C (171 mg) was dissolved in tetrahydrofuran under nitrogen and cooled with iced water. Aqueous tetrabutylammonium fluoride solution (1M, 0.58 mL) was added. The reaction mixture was stirred at 0° C. for 1 hour. The reaction mixture was quenched with aqueous sodium bicarbonate solution and extracted three times with ethyl acetate. The combined organic layers were washed with water and brine, dried over magnesium sulfate, filtrated, and concentrated to provide the crude title compound. MS (APCI) m/z 326.3 (M+H)⁺.

### Example 166E

### tert-butyl (7R,16R)-19,23-dichloro-1-(4-fluorophenyl)-20,22-dimethyl-16-[(4-methylpiperazin-1-yl)methyl]-10-[(2-{4-[2-(tetrahydro-1H-furo[3,4-c]pyrrol-5 (3H)-yl)ethyl]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylate

Example 16N (31 mg), Example 166D (24 mg), triphenylphosphine (55 mg), and N,N,N′,N′-tetramethylazodicarboxamide (38 mg) were combined under argon atmosphere. Tetrahydrofuran (0.5 mL) and toluene (0.5 mL) were added. The reaction mixture was stirred at ambient temperature for 4 days. The reaction mixture was partitioned between dichloromethane and water. The aqueous layer was extracted with dichloromethane another two times. The combined organic extracts were dried over magnesium sulfate, filtrated and concentrated. Purification was performed on a silica gel column (4 g, 0-10% methanol in dichloromethane). The desired fractions were combined and the solvents were removed under reduced pressure to provide the title compound. MS (APCI) m/z 1116.4 (M+H)⁺.

### Example 166F

### (7R,16R)-19,23-dichloro-1-(4-fluorophenyl)-20,22-dimethyl-16-[(4-methylpiperazin-1-yl)methyl]-10-[(2-{4-[2-(tetrahydro-1H-furo[3,4-c]pyrrol-5 (3H)-yl)ethyl]phenyl}pyrimidin-4-yl)methoxy]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 166E (41 mg) was dissolved in dichloromethane (1 mL), trifluoroacetic acid (0.28 mL) was added, and the mixture was stirred overnight at ambient temperature. The reaction mixture was diluted with dichloromethane, cooled with iced water, and washed with sodium bicarbonate solution. The aqueous layer was extracted with dichloromethane another two times. The combined organic extract was dried over magnesium sulfate, filtrated and concentrated. The material was purified by HPLC (Waters XBridge C8 19×150 mm 5 μm column, gradient 5-100% acetonitrile+0.2% ammonium hydroxide in water+0.2% ammonium hydroxide) to yield the title compound. ¹H NMR (600 MHz, dimethylsulfoxide-d₆) δ ppm 8.86 (d, 1H), 8.74 (s, 1H), 8.30-8.29 (m, 2H), 7.50 (d, 1H), 7.37-7.35 (m, 2H), 7.22-7.18 (m, 2H), 7.16-7.12 (m, 2H), 6.88 (d, 1H), 6.75 (dd, 1H), 6.23 (b, 1H), 5.81 (b, 1H), 5.26 (d, 1H), 5.19 (d, 1H), 4.85 (p, 1H), 4.46-4.41 (m, 2H), 3.72 (t, 2H), 3.66 (dd, 1H), 2.98 (dd, 1H), 2.79 (t, 2H), 2.70-2.66 (m, 3H), 2.65-2.61 (m, 3H), 2.57 (b, 2H), 2.51-2.27 (m, 12H), 2.15 (s, 3H), 1.98 (s, 3H), 1.95 (s, 3H). MS (APCI) m/z 1160.3 (M+H)⁺.

### Example 167

### (7R,16R)-19,23-dichloro-1-(4-fluorophenyl)-10-({2-[(1R,5S,6r)-6-hydroxy-3-azabicyclo[3.1.1]heptan-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 167A

### (1R,5S,6s)-3-(4-(hydroxymethyl)pyrimidin-2-yl)-3-azabicyclo[3.1.1]heptan-6-ol

To an 8 mL high pressure reaction vessel, equipped with stir bar, was added 3-azabicyclo[3.1.1]heptan-6-ol hydrochloride (50 mg), (2-chloropyrimidine-4yl)methanol (72 mg), acetonitrile (0.9 mL) and triethylamine (0.14 mL). The flask was capped and the mixture was stirred at 80° C. for 5 hours. After cooling to ambient temperature, the reaction mixture was diluted with dichloromethane and concentrated onto silica gel. Purification by flash chromatography on a CombiFlash® Teledyne Isco system using a Teledyne Isco RediSep® Rf gold 12 g silica gel column (eluting with solvent A=2:1 ethyl acetate:ethanol, solvent B=heptane, 10-100%) afforded the title compound. MS (APCI) m/z 222.4 (M+H)⁺.

### Example 167B

### tert-butyl (7R,16R)-19,23-dichloro-1-(4-fluorophenyl)-10-({2-[(1R,5S,6r)-6-hydroxy-3-azabicyclo[3.1.1]heptan-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 167B was synthesized according to the procedure described for Example 29I, substituting Example 167A for Example 29H. MS (APCI) m/z 1014.9 (M+H)⁺.

### Example 167C

### (7R,16R)-19,23-dichloro-1-(4-fluorophenyl)-10-({2-[(1R,5S,6r)-6-hydroxy-3-azabicyclo[3.1.1]heptan-3-yl]pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 167C was synthesized according to the procedure described for Example 29J, substituting Example 167B for Example 291. ¹H NMR (500 MHz, dimethylsulfoxide-d₆) δ ppm 8.76 (s, 1H), 8.33 (d, 1H), 7.24-7.10 (m, 4H), 6.86-6.77 (m, 2H), 6.70 (d, 1H), 6.30-6.20 (m, 1H), 5.78 (d, 1H), 5.07-4.88 (m, 3H), 4.54-4.36 (m, 2H), 4.02 (t, 1H), 3.78-3.66 (m, 4H), 3.27-2.93 (m, 12H), 2.90-2.84 (m, 1H), 2.80 (s, 3H), 1.97 (d, 6H), 1.66-1.53 (m, 1H), 1.33-1.23 (m, 1H). MS (APCI) m/z 956.3 (M+H)⁺.

### Example 168

### (7R,16R)-19,23-dichloro-1-(4-fluorophenyl)-20,22-dimethyl-16-[(4-methylpiperazin-1-yl)methyl]-10-[(2-{4-[2-(morpholin-4-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 168A

### 4-(2-(4-(4-(((tert-butyldimethylsilyl)oxy)methyl)pyrimidin-2-yl)phenoxy)ethyl)morpholine

A 4 mL vial, equipped with stir bar, was charged with Example 157A (50 mg), 2-morpholinoethan-1-ol (50 μL), triphenylphosphine (120 mg) and (E)-N¹,N¹,N²,N²-tetramethyldiazene-1,2-dicarboxamide (TMAD) (80 mg), and was purged for 30 minutes with argon. A mixture of toluene (1 mL) and tetrahydrofuran (1 mL) was added and the reaction mixture was stirred overnight at ambient temperature. The reaction mixture was concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-5% methanol in dichloromethane) to afford the title compound. MS (APCI) m/z 430.4 (M+H)⁺.

### Example 168B

### (2-(4-(2-morpholinoethoxy)phenyl)pyrimidin-4-yl)methanol

To a solution of Example 168A (59 mg) in tetrahydrofuran (2 mL) was added CsF (60 mg). Subsequently methanol (2 mL) was added and the reaction mixture was stirred overnight at ambient temperature. The reaction mixture was concentrated in vacuo and the residue was purified by normal phase MPLC on a Teledyne-Isco-Combiflash® system (eluting 0-10% methanol in dichloromethane) to afford the title compound. MS (APCI) m/z 316.2 (M+H)⁺.

### Example 168C

### tert-butyl (7R,16R)-19,23-dichloro-1-(4-fluorophenyl)-20,22-dimethyl-16-[(4-methylpiperazin-1-yl)methyl]-10-[(2-{4-[2-(morpholin-4-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (30 mg), Example 168B (25 mg), triphenylphosphine (30 mg) and (E)-N¹,N¹N²,N²-tetramethyldiazene-1,2-dicarboxamide (TMAD) (20 mg), and was purged for 30 minutes with argon. A mixture of toluene (0.5 mL) and tetrahydrofuran (0.5 mL) was added and the reaction mixture was stirred for 5 days at ambient temperature. The reaction mixture was concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-40% methanol in dichloromethane) to afford the title compound. MS (APCI) m/z 1106.5 (M+H)⁺.

### Example 168D

### (7R,16R)-19,23-dichloro-1-(4-fluorophenyl)-20,22-dimethyl-16-[(4-methylpiperazin-1-yl)methyl]-10-[(2-{4-[2-(morpholin-4-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 168C (18 mg) in dichloromethane (1 mL) was added trifluoroacetic acid (100 μL). The reaction mixture was stirred for 2 days at ambient temperature. To the reaction mixture was added trifluoroacetic acid (200 μL) and stirring continued for 3 days at ambient temperature. The reaction mixture was then concentrated in vacuo. The residue was purified by HPLC purification (Waters X-Bridge C8 19×150 mm 5 μm column, gradient 5-100% acetonitrile+0.2% ammonium hydroxide in water+0.2% ammonium hydroxide) provided the title compound. ¹H NMR (600 MHz, dimethylsulfoxide-d₆) δ ppm 8.82 (d, 1H), 8.71 (s, 1H), 8.34 (m, 2H), 7.47 (m, 1H), 7.19 (m, 2H), 7.13 (m, 2H), 7.07 (m, 2H), 6.85 (m, 1H), 6.72 (m, 1H), 6.11 (m, 1H), 5.90 (m, 1H), 5.25 (m, 1H), 5.20 (m, 1H), 4.90 (m, 1H), 4.44 (m, 2H), 4.17 (m, 2H), 3.58 (m, 4H), 2.96 (m, 1H), 2.75-2.25 (m, 17H), 2.14 (s, 3H), 1.99 (s, 3H), 1.96 (s, 3H). MS (APCI) m/z 1051.3 (M+H)⁺.

### Example 169

### (7R,16R)-19,23-dichloro-1-(4-fluorophenyl)-10-({6-[2-(2-methoxyethoxy)ethoxy]-2-(2-methoxyphenyl)pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 169A

### methyl 2-chloro-6-(2-(2-methoxyethoxy)ethoxy)pyrimidine-4-carboxylate

To a solution of 2-(2-methoxyethoxy)ethanol (290 mg) in tetrahydrofuran (8 mL) cooled to 5° C. NaH (126 mg, 60% suspension in paraffin oil) was added and the mixture was stirred for 30 minutes. After cooling to −78° C. a solution of methyl 2,4-dichloropyrimidine-6-carboxylate (500 mg) in tetrahydrofuran (8 mL) was added dropwise and stirring was continued for 16 hours while allowing the mixture to come to ambient temperature. At 5° C., water was added, and the mixture was extracted twice with ethyl acetate (20 mL). The combined organic extracts were washed with brine, dried over magnesium sulfate, filtered and concentrated in vacuo. Purification by chromatography using an ISCO CombiFlash® Companion MPLC (4 g Chromabond® SiOH column, eluting with 0-50% heptane/ethyl acetate) provided the title compound. MS (APCI) m/z 291.2 (M+H)⁺.

### Example 169B

### methyl 6-(2-(2-methoxyethoxy)ethoxy)-2-(2-methoxyphenyl)pyrimidine-4-carboxylate

A 10 mL microwave tube was charged with Example 169A (150 mg), 2-methoxyphenylboronic acid (80 mg) and dioxane (2 mL), and the solution degassed with nitrogen. The vial was transferred into a glove box, then 1,1′-bis(diphenylphosphino)ferrocene-palladium (II) dichloride dichloromethane complex (17.9 mg) and CsF (167 mg) were added. The vial was capped and heated in a Biotage® Initiator microwave for 2 hours to 80° C. Water (20 mL) and ethyl acetate (20 mL) were added, and the material was filtered off and washed with ethyl acetate and water. The layers were separated and the aqueous layer was extracted once more with ethyl acetate (15 mL). The combined organic layers were washed with brine, dried over magnesium sulfate, filtered and concentrated in vacuo. Purification by chromatography using an ISCO CombiFlash® Companion MPLC (5 g Chromabond® SiOH column, eluting with 0-50% heptane/ethyl acetate) gave the title compound. MS (APCI) m/z 363.2 (M+H)⁺.

### Example 169C

### (6-(2-(2-methoxyethoxy)ethoxy)-2-(2-methoxyphenyl)pyrimidin-4-yl)methanol

To a solution of Example 169B (150 mg) in methanol (10 mL). NaBH₄ (55 mg) was added and the reaction mixture was stirred at ambient temperature for 1 hour. Water (40 mL) was added and the mixture was extracted twice with dichloromethane (20 mL). The combined organic layers were washed with brine, dried over magnesium sulfate, filtered and concentrated in vacuo. Purification by chromatography using an ISCO CombiFlash® Companion MPLC (5 g Chromabond® SiOH column, eluting with 0-5% dichloromethane/methanol) gave the title compound. MS (APCI) m/z 335.2 (M+H)⁺.

### Example 169D

### tert-butyl (7R,16R)-19,23-dichloro-1-(4-fluorophenyl)-10-({6-[2-(2-methoxyethoxy)ethoxy]-2-(2-methoxyphenyl)pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 89C by replacing Example 89B with Example 169C. Purification by chromatography using an ISCO CombiFlash® Companion MPLC (4 g RediSep® Gold column, eluting with 7-10% dichloromethane/methanol) provided title compound. MS (APCI) m/z 1125.4 (M+H)⁺.

### Example 169E

### (7R,16R)-19,23-dichloro-1-(4-fluorophenyl)-10-({6-[2-(2-methoxyethoxy)ethoxy]-2-(2-methoxyphenyl)pyrimidin-4-yl}methoxy)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

The title compound was prepared as described in Example 89D by replacing Example 89C with Example 169D. Purification by HPLC (Waters XBridge C8 19×150 mm 5 μm column, gradient 5-100% acetonitrile+0.2% ammonium hydroxide in water+0.2% ammonium hydroxide) provided the title compound. ¹H NMR (600 MHz, dimethylsulfoxide-d₆) δ ppm 13.06 (bs, 1H), 8.74 (s, 1H), 7.60 (dd, 1H), 7.46 (m, 1H), 7.24-7.17 (m, 2H), 7.19-7.10 (m, 3H), 7.04 (td, 1H), 6.88 (d, 1H), 6.85 (s, 1H), 6.75 (dd, 1H), 6.23 (m, 1H), 5.81 (m, 1H), 5.13 (d, 1H), 5.05 (d, 1H), 4.89 (m, 1H), 4.46 (m, 4H), 3.79 (s, 3H), 3.76 (m, 2H), 3.63-3.54 (m, 3H), 3.43 (m, 2H), 3.22 (s, 3H), 2.99 (dd, 1H), 2.70 (dd, 1H), 2.66 (dd, 1H), 2.55-2.25 (m, 8H), 2.16 (s, 3H), 1.99 (s, 3H), 1.96 (s, 3H). MS (APCI) m/z 1069.3 (M+H)⁺.

### Example 170

### (7R,16R)-19,23-dichloro-1-(4-fluorophenyl)-20,22-dimethyl-16-[(4-methylpiperazin-1-yl)methyl]-10-[(2-{4-[2-(8-oxa-3-azabicyclo[3.2.1]octan-3-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 170A

### 2-(8-oxa-3-azabicyclo[3.2.1]octan-3-yl)ethyl methanesulfonate

2-(8-Oxa-3-azabicyclo[3.2.1]octan-3-yl)ethanol, hydrochloric acid (50 mg) was dissolved in dichloromethane (3 mL) under a nitrogen atmosphere and cooled to 0° C. with iced water. Triethylamine (108 μL) and methanesulfonyl chloride (24 μL) were added and the reaction mixture was stirred at 0° C. for 2 hours. To the reaction mixture was added dichloromethane and the organic phase was extracted with water. After phase separation via a Chromabond® PTS cartridge, the organic phase was concentrated in vacuo. The crude material was used without any further purification in the next step. MS (APCI) m/z 236.20 (M+H)⁺.

### Example 170B

### (1R,5S)-3-(2-(4-(4-(((tert-butyldimethylsilyl)oxy)methyl)pyrimidin-2-yl)phenoxy)ethyl)-8-oxa-3-azabicyclo[3.2.1]octane

A round bottom flask, equipped with stir bar, was charged with Example 157A (50 mg) and N,N-dimethylformamide (1 mL). Example 170A (87 mg) and subsequently Cs₂CO₃ (154 mg) were added. The reaction mixture was stirred overnight at ambient temperature. The reaction mixture was concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting 0-10% methanol in dichloromethane) to afford the title compound. MS (APCI) m/z 456.40 (M+H)⁺.

### Example 170C

### (2-(4-(2-((1R,5S)-8-oxa-3-azabicyclo[3.2.1]octan-3-yl)ethoxy)phenyl)pyrimidin-4-yl)methanol

To a solution of Example 170B (60 mg) in tetrahydrofuran (1 mL) was added CsF (50 mg). Subsequently methanol (1 mL) was added and the reaction mixture was stirred overnight at ambient temperature. The reaction mixture was concentrated in vacuo and to the residue was added dichloromethane. The precipitate was filtered off and the organic phase was concentrated in vacuo. The residue was purified by normal phase MPLC on a Teledyne-Isco-Combiflash® system (eluting 0-10% methanol in dichloromethane) to afford the title compound. MS (APCI) m/z 342.20 (M+H)⁺.

### Example 170D

### tert-butyl (7R,16R)-19,23-dichloro-1-(4-fluorophenyl)-20,22-dimethyl-16-[(4-methylpiperazin-1-yl)methyl]-10-[(2-{4-[2-(8-oxa-3-azabicyclo[3.2.1]octan-3-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (30 mg), Example 170C (25 mg), triphenylphosphine (30 mg) and (E)-N¹,N¹,N²,N²-tetramethyldiazene-1,2-dicarboxamide (TMAD) (20 mg) and was purged for 30 minutes with argon. A mixture of toluene (0.5 mL) and tetrahydrofuran (0.5 mL) was added and the reaction mixture was stirred for 5 days at ambient temperature. The reaction mixture was concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-30% methanol in dichloromethane) to afford the title compound. MS (APCI) m/z 1132.40 (M+H)⁺.

### Example 170E

### (7R,16R)-19,23-dichloro-1-(4-fluorophenyl)-20,22-dimethyl-16-[(4-methylpiperazin-1-yl)methyl]-10-[(2-{4-[2-(8-oxa-3-azabicyclo[3.2.1]octan-3-yl)ethoxy]phenyl}pyrimidin-4-yl)methoxy]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 170D (35 mg) in dichloromethane (1 mL) was added trifluoroacetic acid (150 μL). The reaction mixture was stirred for 2 days at ambient temperature. The reaction mixture was then concentrated in vacuo. The residue was purified by HPLC Purification (Waters X-Bridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.82 (d, 1H), 8.74 (s, 1H), 8.33 (m, 2H), 7.44 (m, 1H), 7.17 (m, 2H), 7.13 (m, 2H), 7.07 (m, 2H), 6.88 (m, 1H), 6.75 (m, 1H), 6.23 (m, 1H), 5.80 (m, 1H), 5.24 (d, 1H), 5.17 (d, 1H), 4.86 (m, 1H), 4.44 (m, 2H), 4.20 (m, 2H), 4.14 (m, 2H), 3.64 (m, 1H), 2.98 (m, 1H) 2.67 (m, 6H), 2.60-2.25 (m, 10H), 2.15 (s, 3H), 2.00 (s, 3H), 1.97 (s, 3H), 1.81 (m, 2H), 1.69 (m, 2H). MS (APCI) m/z 1077.30 (M+H)⁺.

### Example 171

### (7R,16R)-10-{[2-(3-azabicyclo[3.1.1]heptan-3-yl)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 171A

### (2-(3-azabicyclo[3.1.1]heptan-3-yl)pyrimidin-4-yl)methanol

Example 171A was synthesized according to the procedure described for Example 167A, substituting 3-azabicyclo[3.1.1]heptane hydrochloride for azabicyclo[3.1.1]heptan-6-ol hydrochloride. MS (APCI) m/z 205.9 (M+H)⁺.

### Example 171B

### tert-butyl (7R,16R)-10-{[2-(3-azabicyclo[3.1.1]heptan-3-yl)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 171B was synthesized according to the procedure described for Example 29I, substituting Example 171A for Example 29H. MS (APCI) m/z 997.0 (M+H)⁺.

### Example 171C

### (7R,16R)-10-{[2-(3-azabicyclo[3.1.1]heptan-3-yl)pyrimidin-4-yl]methoxy}-19,23-dichloro-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 171C was synthesized according to the procedure described for Example 29J, substituting Example 171B for Example 291. ¹H NMR (500 MHz, dimethyl sulfoxide-d₆) δ ppm 8.72 (s, 1H), 8.34 (d, 1H), 7.21-7.15 (m, 2H), 7.15-7.10 (m, 2H), 6.80 (d, 1H), 6.74 (d, 1H), 6.72 (dd, 1H), 6.22 (dd, 1H), 5.82 (d, 1H), 5.03-4.90 (m, 2H), 4.90-4.80 (m, 1H), 4.43 (d, 2H), 3.72-3.65 (m, 6H), 3.64-3.57 (m, 1H), 2.94 (dd, 1H), 2.75-2.60 (m, 2H), 2.55-2.39 (m, 8H), 2.24 (s, 3H), 2.17-2.12 (m, 2H), 1.96 (s, 6H), 1.33 (d, 2H). MS (APCI) m/z 942.8 (M+H)⁺.

### Example 172

### (7R,16R)-19,23-dichloro-10-({2-[(1R,5S)-6,6-difluoro-3-azabicyclo[3.1.1]heptan-3-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 172A

### (2-(6,6-difluoro-3-azabicyclo[3.1.1]heptan-3-yl)pyrimidin-4-yl)methanol

Example 172A was synthesized according to the procedure described for Example 167A, substituting 6,6-difluoro-3-azabicyclo[3.1.1]heptane hydrochloride for azabicyclo[3.1.1]heptan-6-ol hydrochloride. MS (APCI) m/z 242.3 (M+H)⁺.

### Example 172B

### tert-butyl (7R,16R)-19,23-dichloro-10-({2-[(1R,5S)-6,6-difluoro-3-azabicyclo[3.1.1]heptan-3-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 172B was synthesized according to the procedure described for Example 291, substituting Example 172A for Example 29H. MS (APCI) m/z 1033.1 (M+H)⁺.

### Example 172C

### (7R,16R)-19,23-dichloro-10-({2-[(1R,5S)-6,6-difluoro-3-azabicyclo[3.1.1]heptan-3-yl]pyrimidin-4-yl}methoxy)-1-(4-fluorophenyl)-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

Example 172C was synthesized according to the procedure described for Example 29J, substituting Example 172B for Example 291. ¹H NMR (500 MHz, dimethylsulfoxide-d₆) δ ppm 8.73 (s, 1H), 8.37 (d, 1H), 7.23-7.16 (m, 2H), 7.16-7.10 (m, 2H), 6.85-6.78 (m, 2H), 6.73 (dd, 1H), 6.22 (dd, 1H), 5.81 (d, 1H), 5.06-4.89 (m, 2H), 4.86 (p, 1H), 4.43 (d, 2H), 4.04 (d, 2H), 3.67 (d, 2H), 3.62-3.57 (m, 1H), 3.06-2.88 (m, 3H), 2.74-2.60 (m, 2H), 2.56-2.38 (m, 8H), 2.23 (s, 3H), 2.04-1.99 (m, 1H), 1.96 (d, 6H), 1.56 (dd, 1H). MS (APCI) m/z 978.9 (M+H)⁺.

### Example 173

### (7R,16R)-19,23-dichloro-1-(4-fluorophenyl)-20,22-dimethyl-10-{[2-(4-{[(2S)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 173A

### 2-((4-(4-(((tert-butyldimethylsilyl)oxy)methyl)pyrimidin-2-yl)phenoxy)methyl)-4-methyl-1,4-oxazepane

A 4 mL vial, equipped with stir bar, was charged with Example 157A (120 mg), (4-methyl-1,4-oxazepan-2-yl)methanol (90 mg), triphenylphosphine (250 mg) and (E)-N¹,N¹,N²,N²-tetramethyldiazene-1,2-dicarboxamide (TMAD) (150 mg), and was purged for 30 minutes with argon. A mixture of toluene (1 mL) and tetrahydrofuran (1 mL) was added and the reaction mixture was stirred for 5 days at ambient temperature. The reaction mixture was concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-10% methanol in dichloromethane) to afford the title compound. MS (APCI) m/z 444.2 (M+H)⁺.

### Example 173B

### (S)-(2-(4-((4-methyl-1,4-oxazepan-2-yl)methoxy)phenyl)pyrimidin-4-yl)methanol

To a solution of Example 173A (153 mg) in tetrahydrofuran (2 mL) was added CsF (131 mg). Subsequently methanol (2 mL) was added and the reaction mixture was stirred overnight at ambient temperature. The reaction mixture was concentrated in vacuo. To the residue was added dichloromethane and the organic phase was filtered through a Chromabond® PTS cartridge. The organic phase was concentrated in vacuo and the residue was purified by normal phase MPLC on a Teledyne-Isco-Combiflash® system (eluting with 0-20% methanol in dichloromethane). Chiral separation of the product by SFC (Chiralpak IA, 250×20 mm, 5 μm column, isocratic, 70% liquid CO₂+30% methanol+0.2% ammonium hydroxide in water) provided the title compound as the earlier-eluting enantiomer. The chirality was arbitrarily assigned. MS (APCI) m/z 329.2 (M+H)⁺.

### Example 173C

### (R)-(2-(4-((4-methyl-1,4-oxazepan-2-yl)methoxy)phenyl)pyrimidin-4-yl)methanol

The title compound was isolated as the later-eluting enantiomer from Example 173B. The chirality was arbitrarily assigned.

### Example 173D

### tert-butyl (7R,16R)-19,23-dichloro-1-(4-fluorophenyl)-20,22-dimethyl-10-{[2-(4-{[(2S)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (35 mg), Example 173B (20 mg), triphenylphosphine (30 mg) and (E)-N¹,N¹,N²,N²-tetramethyldiazene-1,2-dicarboxamide (TMAD) (20 mg), and was purged for 30 minutes with argon. A mixture of toluene (0.5 mL) and tetrahydrofuran (0.5 mL) was added and the reaction mixture was stirred overnight at ambient temperature. The reaction mixture was concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-40% methanol in dichloromethane) to afford the title compound. MS (APCI) m/z 1120.2 (M+H)⁺.

### Example 173E

### (7R,16R)-19,23-dichloro-1-(4-fluorophenyl)-20,22-dimethyl-10-{[2-(4-{[(2S)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 173D (23 mg) in dichloromethane (1 mL) was added trifluoroacetic acid (120 μL). The reaction mixture was stirred for 5 days at ambient temperature. The reaction mixture was then concentrated in vacuo. Purification by HPLC (Waters X-Bridge C18 19×150 mm 5 μm column, gradient 5-95% acetonitrile+0.1% trifluoroacetic acid in water+0.1% trifluoroacetic acid) provided the title compound. ¹H NMR (600 MHz, dimethylsulfoxide-d₆) δ ppm 8.82 (d, 1H), 8.74 (s, 1H), 8.33 (m, 2H), 7.44 (m, 1H), 7.20 (m, 2H), 7.14 (m, 2H), 7.05 (m, 2H), 6.88 (m, 1H), 6.74 (m, 1H), 6.23 (m, 1H), 5.81 (m, 1H), 5.21 (m, 1H), 5.15 (m, 1H), 4.86 (m, 1H), 4.44 (m, 2H), 4.00-3.90 (m, 3H), 3.77 (m, 2H), 3.65 (m, 1H), 2.98 (m, 1H), 2.88 (m, 1H), 2.70-2.55 (m, 3H), 2.50-2.25 (m, 13H), 2.15 (s, 3H), 2.00 (s, 3H), 1.97 (s, 3H), 1.83 (m, 1H), 1.73 (m, 1H). MS (APCI) m/z 1065.05 (M+H)⁺.

### Example 174

### (7R,16R)-19,23-dichloro-1-(4-fluorophenyl)-10-{[2-(4-{[(3S,3aR,6S,6aR)-6-hydroxyhexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 174A

### tert-butyl (7R,16R)-19,23-dichloro-1-(4-fluorophenyl)-20,22-dimethyl-16-[(4-methylpiperazin-1-yl)methyl]-10-({2-[4-({(3S,3aR,6S,6aR)-6-[(4-nitrobenzoyl)oxy]hexahydrofuro[3,2-b]furan-3-yl}oxy)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 161G (66 mg) and 4-nitrobenzoic acid (34 mg) in tetrahydrofuran (294 μL) at ambient temperature was added triphenylphosphine (54 mg) followed by di-tert-butyl azodicarboxylate (47 mg), and the reaction was allowed to stir overnight. The reaction mixture was diluted with ethyl acetate, filtered over diatomaceous earth and concentrated. The residue was purified by normal phase MPLC on a Teledyne Isco Combiflash® Rf+12 g gold silica gel column eluting with 0-8% methanol in dichloromethane to give the title compound.

### Example 174B

### (7R,16R)-19,23-dichloro-1-(4-fluorophenyl)-20,22-dimethyl-16-[(4-methylpiperazin-1-yl)methyl]-10-({2-[4-({(3S,3aR,6S,6aR)-6-[(4-nitrobenzoyl)oxy]hexahydrofuro[3,2-b]furan-3-yl}oxy)phenyl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 174A (71 mg) in dichloromethane (280 μL) was added trifluoroacetic acid (280 μL), and the reaction was allowed to stir overnight. The reaction was concentrated under a stream of nitrogen and was taken up in water and acetonitrile. The mixture was purified by RP-HPLC on a Gilson PLC 2020 using a Luna™ column (250×50 mm, 10 mm, 30-90% over 30 minutes with acetonitrile in water containing 10 mM ammonium acetate then 30-90% over 30 minutes with acetonitrile in water containing 0.01% trifluoroacetic acid) to give the title compound.

### Example 174C

### (7R,16R)-19,23-dichloro-1-(4-fluorophenyl)-10-{[2-(4-{[(3S,3aR,6S,6aR)-6-hydroxyhexahydrofuro[3,2-b]furan-3-yl]oxy}phenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 174B (14 mg) in tetrahydrofuran (160 μL) and methanol (160 μL) at ambient temperature was added a solution of lithium hydroxide (6.9 mg) in water (160 μL), and the reaction was allowed to stir at ambient temperature. The reaction was quenched with trifluoroacetic acid (27 μL), taken up in dimethylsulfoxide and was purified by RP-HPLC on a Gilson PLC 2020 using a Luna™ column (250×50 mm, 10 mm, 30-80% over 30 minutes with acetonitrile in water containing 10 mM ammonium acetate) to give the title compound. ¹H NMR (400 MHz, dimethylsulfoxide-d₆) δ ppm 8.82 (d, 1H), 8.68 (s, 1H), 8.40-8.30 (m, 2H), 7.50 (d, 1H), 7.24-7.05 (m, 7H), 6.82 (d, 1H), 6.72-6.63 (m, 1H), 6.18-6.08 (m, 1H), 5.95-5.87 (m, 1H), 5.35-5.11 (m, 3H), 4.98-4.85 (m, 2H), 4.61 (d, 1H), 4.50-4.36 (m, 3H), 4.17-4.10 (m, 1H), 3.98 (dd, 1H), 3.88 (d, 1H), 3.80 (dd, 1H), 3.71 (d, 1H), 3.62-3.52 (m, 1H), 3.00-2.89 (m, 1H), 2.73-2.58 (m, 2H), 2.48-2.22 (m, 6H), 2.14 (s, 3H), 2.00 (s, 3H), 1.92 (s, 3H). MS (ESI) m/z 1062.9 (M−H)⁻.

### Example 175

### (7R,16R)-19,23-dichloro-1-(4-fluorophenyl)-20,22-dimethyl-10-{[2-(4-{[(2R)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 175A

### tert-butyl (7R,16R)-19,23-dichloro-1-(4-fluorophenyl)-20,22-dimethyl-10-{[2-(4-{[(2R)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (25 mg), Example 173C (12 mg), triphenylphosphine (25 mg) and (E)-N¹,N¹,N²,N²-tetramethyldiazene-1,2-dicarboxamide (TMAD) (15 mg), and was purged for 30 minutes with argon. A mixture of toluene (0.5 mL) and tetrahydrofuran (0.5 mL) was added and the reaction mixture was stirred for 3 days at ambient temperature. The reaction mixture was concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting with 0-60% methanol in dichloromethane) to afford the title compound. MS (APCI) m/z 1120.25 (M+H)⁺.

### Example 175B

### (7R,16R)-19,23-dichloro-1-(4-fluorophenyl)-20,22-dimethyl-10-{[2-(4-{[(2R)-4-methyl-1,4-oxazepan-2-yl]methoxy}phenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 175A (24 mg) in dichloromethane (1 mL) was added trifluoroacetic acid (150 μL). The reaction mixture was stirred overnight at ambient temperature. The reaction mixture was then concentrated in vacuo. Purification by HPLC (Waters X-Bridge C18 19×150 mm 5 μm column, gradient 5-95% acetonitrile+0.1% trifluoroacetic acid in water+0.1% trifluoroacetic acid) provided the title compound as a trifluoroacetic acid salt. The residue was dissolved in dichloromethane (5 mL) and saturated aqueous NaHCO₃-solution was added. The reaction mixture was stirred for 30 minutes at ambient temperature. The phases were separated with a Horizon DryDisk® and the organic phase was concentrated in vacuo to provide the title compound. ¹H NMR (600 MHz, dimethylsulfoxide-d₆) δ ppm 8.75 (m, 2H), 8.30 (m, 2H), 7.45 (m, 1H), 7.20 (m, 2H), 7.14 (m, 2H), 7.00-6.90 (m, 3H), 6.77 (m, 1H), 6.25 (m, 1H), 5.84 (m, 1H), 5.25-5.15 (m, 2H), 4.86 (m, 1H), 4.46 (m, 2H), 3.90-3.65 (m, 6H), 2.94 (m, 2H), 2.77 (m, 1H), 2.67 (m, 2H), 2.60-2.25 (m, 13H), 2.15 (s, 3H), 1.99 (s, 3H), 1.96 (s, 3H), 1.87 (m, 1H), 1.73 (m, 1H). MS (APCI) m/z 1065.3 (M+H)⁺.

### Example 176

### (7R,16R)-19,23-dichloro-1-(4-fluorophenyl)-10-[(6-{2-[2-(2-methoxyethoxy)ethoxy]ethoxy}pyrazin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 176A

### tert-butyl (7R,16R)-19,23-dichloro-1-(4-fluorophenyl)-10-[(6-{2-[2-(2-methoxyethoxy)ethoxy]ethoxy}pyrazin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (30 mg), (6-(2-[2-(2-methoxyethoxy)ethoxy]ethoxy) pyrazine-2-yl)methanol (15 mg), triphenylphosphine (25 mg) and (E)-N¹,N¹,N²,N²-tetramethyldiazene-1,2-dicarboxamide (TMAD) (15 mg), and was purged for 30 minutes with argon. A mixture of toluene (0.5 mL) and tetrahydrofuran (0.5 mL) was added and the reaction mixture was stirred for 3 days at ambient temperature. The reaction mixture was then concentrated in vacuo. The residue was dissolved in dichloromethane and the organic phase was extracted with water. After phase separation via a Chromabond® PTS cartridge, the organic phase was concentrated in vacuo. The residue was purified by normal phase MPLC on a Teledyne-Isco-Combiflash® system (eluting 0-60% methanol in dichloromethane) to afford the title compound. MS (APCI) m/z 1063.15 (M+H)⁺.

### Example 176B

### (7R,16R)-19,23-dichloro-1-(4-fluorophenyl)-10-[(6-{2-[2-(2-methoxyethoxy)ethoxy]ethoxy}pyrazin-2-yl)methoxy]-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 176A (30.8 mg) in dichloromethane (1 mL) was added trifluoroacetic acid (150 μL). The reaction mixture was stirred overnight at ambient temperature. The reaction mixture was then concentrated in vacuo. The residue was purified by HPLC (Waters X-Bridge C18 19×150 mm 5 μm column, gradient 5-95% acetonitrile+0.1% trifluoroacetic acid in water+0.1% trifluoroacetic acid) to provide the title compound as a trifluoroacetic acid salt. The residue was dissolved in dichloromethane (5 mL) and saturated aqueous NaHCO₃ solution was added. The reaction mixture was stirred for 30 minutes at ambient temperature. The phases were separated with a Horizon DryDisk® and the organic phase was concentrated in vacuo to provide the title compound. ¹H NMR (600 MHz, dimethylsulfoxide-d₆) δ ppm 8.74 (s, 1H), 8.31 (s, 1H), 8.27 (s, 1H), 7.20 (m, 2H), 7.14 (m, 2H), 6.93 (m, 1H), 6.77 (m, 1H), 6.20 (m, 1H), 5.78 (m, 1H), 5.15 (d, 1H), 5.10 (d, 1H), 4.88 (m, 1H), 4.43 (m, 4H), 3.76 (m, 2H), 3.55-3.45 (m, 7H), 3.40 (m, 2H), 3.21 (s, 3H), 2.93 (m, 1H), 2.69 (m, 2H), 2.50-2.25 (m, 8H), 2.19 (s, 3H), 1.97 (s, 3H), 1.96 (s, 3H). MS (APCI) m/z 1008.2 (M+H)⁺.

### Example 177

### (7R,16R)-19,23-dichloro-1-(4-fluorophenyl)-20,22-dimethyl-10-[(2-{[(2S)-4-methyl-1,4-oxazepan-2-yl]methoxy}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

### Example 177A

### (S)-2-(((4-(((tert-butyldimethylsilyl)oxy)methyl)pyrimidin-2-yl)oxy)methyl)-4-methyl-1,4-oxazepane

NaH (34.7 mg, 50% in mineral oil) was suspended in tetrahydrofuran (0.5 mL). (S)-(4-Methyl-1,4-oxazepan-2-yl)methanol (70 mg) was dissolved in tetrahydrofuran (1 mL) and slowly added to the reaction mixture dropwise within 5 minutes at 5° C. The reaction mixture was stirred for 1 hour at 5° C. 4-(((tert-Butyldimethylsilyl)oxy)methyl)-2-chloropyrimidine was dissolved in tetrahydrofuran (2 mL) and slowly added to the reaction mixture dropwise within 5 minutes at 5° C. The reaction mixture was allowed to warm to ambient temperature and was stirred overnight. The reaction mixture was then stirred for 1 hour at 40° C. Dichloromethane was added and saturated aqueous NaHCO₃ solution was added to the reaction mixture and stirring was continued for 5 minutes at ambient temperature. The phases were separated with a Horizon DryDisk® and the organic phase was concentrated in vacuo. The residue was purified by normal phase MPLC on a Teledyne-Isco-Combiflash® system (eluting with 0-10% methanol in dichloromethane) to afford the title compound. MS (APCI) m/z 368.2 (M+H)⁺.

### Example 177B

### (S)-(2-((4-methyl-1,4-oxazepan-2-yl)methoxy)pyrimidin-4-yl)methanol

To a solution of Example 177A (100 mg) in tetrahydrofuran (0.9 mL) was added CsF (103 mg). Subsequently methanol (0.45 mL) was added and the reaction mixture was stirred for 2 days at ambient temperature. The reaction mixture was concentrated in vacuo. The residue was purified by normal phase MPLC on a Teledyne-Isco-Combiflash® system (Flashpure ALOX neutral; eluting 0-5% methanol in dichloromethane) to afford the title compound. MS (APCI) m/z 254.20 (M+H)⁺.

### Example 177C

### tert-butyl (7R,16R)-19,23-dichloro-1-(4-fluorophenyl)-20,22-dimethyl-10-[(2-{[(2S)-4-methyl-1,4-oxazepan-2-yl]methoxy}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 16N (35 mg), Example 177B (13.2 mg), triphenylphosphine (45.3 mg) and (E)-N¹,N¹,N²,N²-tetramethyldiazene-1,2-dicarboxamide (TMAD) (29.8 mg), and was purged for 30 minutes with argon. Toluene (0.74 mL) was added and the reaction mixture was stirred for 20 hours at ambient temperature. The reaction mixture was concentrated in vacuo. The residue was dissolved in dichloromethane and the organic phase was extracted with water and brine. The organic phase was dried via Horizon DryDisk® and was concentrated in vacuo. The residue was purified by normal phase MPLC on a Teledyne-Isco-Combiflash® system (eluting 0-20% methanol in dichloromethane) to afford the title compound. MS (APCI) m/z 1045.2 (M+H)⁺.

### Example 177D

### (7R,16R)-19,23-dichloro-1-(4-fluorophenyl)-20,22-dimethyl-10-[(2-{[(2S)-4-methyl-1,4-oxazepan-2-yl]methoxy}pyrimidin-4-yl)methoxy]-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic Acid

To a solution of Example 177C (31 mg) in dichloromethane (406 μL) was added trifluoroacetic acid (229 μL). The reaction mixture was stirred for 10 hours at ambient temperature. The reaction mixture was concentrated in vacuo. The residue was dissolved in dichloromethane and saturated aqueous NaHCO₃ solution was added. The aqueous phase was extracted twice with dichloromethane. The combined organic extracts were dried via Horizon DryDisk® and concentrated in vacuo. The residue was purified by HPLC (Waters X-Bridge C18 19×150 mm 5 μm column, gradient 5-95% acetonitrile+0.1% trifluoroacetic acid in water+0.1% trifluoroacetic acid) to provide the title compound as a trifluoroacetic acid salt. The residue was dissolved in dichloromethane and saturated aqueous NaHCO₃ solution was added. The aqueous phase was extracted twice with dichloromethane. The combined organic extracts were dried via Horizon DryDisk® and concentrated in vacuo to provide the title compound. ¹H NMR (600 MHz, dimethylsulfoxide-d₆) δ ppm 8.72 (s, 1H), 8.58 (d, 1H), 7.25 (d, 1H), 7.20 (m, 2H), 7.14 (m, 2H), 6.78 (m, 1H), 6.73 (m, 1H), 6.16 (m, 1H), 5.81 (m, 1H), 5.15 (d, 1H), 5.08 (d, 1H), 4.86 (m, 1H), 4.44 (m, 2H), 4.14 (m, 2H), 3.94 (m, 1H), 3.71 (m, 1H), 3.60 (m, 1H), 3.52 (m, 1H), 2.92 (m, 2H), 2.87 (m, 1H), 2.75 (m, 2H), 2.70-2.40 (m, 10H), 2.35 (s, 3H), 2.17 (s, 3H), 2.00 (s, 3H), 1.97 (s, 3H): 1.80-1.70 (m 2H). MS (APCI) m/z 989.2 (M+H)⁺.

### Example 178

### (7R,16R)-19,23-dichloro-1-(4-fluorophenyl)-10-({3-[2-(2-methoxyethoxy)ethoxy]-6-(2-methoxyphenyl)pyridin-2-yl}methoxy)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

### Example 178A

### Methyl 3-hydroxy-6-(2-methoxyphenyl)picolinate

2-(2-Methoxyphenyl)-4,4,5,5-tetramethyl-1,3,2-dioxaborolane (524 mg), methyl 6-chloro-3-hydroxypicolinate (400 mg) and 1,1′-bis(diphenylphosphino)ferrocene-palladium (II) dichloride dichloromethane complex (139 mg) were combined and flushed with argon for 5 minutes. 1,4-Dioxane (11 mL, degassed with argon) and aqueous sodium carbonate solution (2 M, 3.20 mL, degassed with argon) were added. The reaction mixture was heated at 120° C. in a Biotage® Initiator microwave reactor for 4 hours. The reaction mixture was diluted with dichloromethane and washed with water. The aqueous layer was washed with dichloromethane (twice) and acidified with hydrochloric acid (1 M) to pH 2. The aqueous layer was extracted with dichloromethane (three times). The organic layer was dried by a PTS-cartridge and concentrated to yield the title compound. MS (ESI) m/z 260.4 (M+H)⁺.

### Example 178B

### methyl 3-(2-(2-methoxyethoxy)ethoxy)-6-(2-methoxyphenyl)picolinate

Example 178A (150 mg) and cesium carbonate (754 mg) were suspended in N,N-dimethyl formamide (2.0 mL). 1-Bromo-2-(2-methoxyethoxy) ethane (371 mg) was added. The reaction mixture was stirred at 25° C. for 3 days. The reaction mixture was diluted with water and ethyl acetate. The phases were separated and the aqueous layer was extracted with ethyl acetate (three times). The combined organic phases were dried over sodium sulfate, filtered, and concentrated. Purification was performed on a silica gel column (4 g, 0-30% methanol in dichloromethane). The desired fractions were combined and the solvents were removed under reduced pressure to provide the title compound. MS (APCI) m/z 362.2 (M+H)⁺.

### Example 178C

### (3-(2-(2-methoxyethoxy)ethoxy)-6-(2-methoxyphenyl)pyridin-2-yl)methanol

Example 178B (72 mg) was dissolved in tetrahydrofuran (2.0 mL) and cooled by an ice-bath to 0° C. Lithium aluminum hydride (1M in tetrahydrofuran, 0.40 mL) was added dropwise. The reaction mixture was stirred for 10 minutes while warming up to ambient temperature. The reaction mixture was diluted with dichloromethane and water. The phases were separated. The organic phase was dried over sodium sulfate, filtrated and concentrated. Purification was performed on a silica gel column (4 g, 0-30% methanol in dichloromethane). The desired fractions were combined and the solvents were removed under reduced pressure to provide the title compound. MS (APCI) m/z 334.1 (M+H)⁺.

### Example 178D

### tert-butyl (7R,16R)-19,23-dichloro-1-(4-fluorophenyl)-10-({3-[2-(2-methoxyethoxy)ethoxy]-6-(2-methoxyphenyl)pyridin-2-yl}methoxy)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 178C (15 mg), Example 16N (25 mg), triphenylphosphine (28 mgl) and N,N,N′,N′-tetramethylazodicarboxamide (19 mg) were combined and flushed with argon for 15 minutes. Toluene (0.7 mL, flushed with argon) was added and the reaction mixture was stirred overnight at ambient temperature. The reaction mixture was concentrated. Purification was performed on a silica gel column (4 g, 0-30% methanol in dichloromethane). The desired fractions were combined and the solvents were removed under reduced pressure to provide the title compound. MS (APCI) m/z 1124.2 (M+H)⁺.

### Example 178E

### (7R,16R)-19,23-dichloro-1-(4-fluorophenyl)-10-({3-[2-(2-methoxyethoxy)ethoxy]-6-(2-methoxyphenyl)pyridin-2-yl}methoxy)-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic Acid

Example 178D (57 mg) was dissolved in dichloromethane (1 mL), trifluoroacetic acid (0.32 mL) was added, and the reaction mixture was stirred at ambient temperature for 6 hours. All volatiles were evaporated and the material was purified by HPLC (Waters XBridge C8 19×150 mm 5 μm column, gradient 5-100% acetonitrile+0.2% ammonium hydroxide in water+0.2% ammonium hydroxide) to provide the title compound. ¹H NMR (600 MHz, dimethylsulfoxide-d₆) δ ppm 8.67 (s, 1H), 7.82 (d, 1H), 7.72 (dd, 1H), 7.53 (d, 1H), 7.35-7.32 (m, 1H), 7.20-7.17 (m, 2H), 7.12-7.10 (m, 3H), 7.01-6.98 (m, 2H), 6.69 (d, 1H), 6.10 (b, 1H), 5.85 (b, 1H), 5.13 (d, 1H), 5.09 (d, 1H), 4.96 (b, 1H), 4.47 (d, 1H), 4.40 (dd, 1H), 4.22 (h, 2H), 3.83 (s, 3H), 3.79-3.74 (m, 2H), 3.57-3.56 (m, 2H), 3.40-3.39 (m, 2H), 3.19 (s, 3H), 2.85-2.82 (d, 1H), 2.71-2.63 (m, 3H), 2.54-2.27 (m, 8H), 2.16 (s, 3H), 1.98 (s, 3H), 1.87 (s, 3H). MS (APCI) m/z 1068.2 (M+H)⁺.

### BIOLOGICAL EXAMPLES

### Exemplary MCL-1 Inhibitors Bind MCL-1

The ability of the exemplary MCL-1 inhibitors of Examples 1 through 178 to bind MCL-1 was demonstrated using the Time Resolved-Fluorescence Resonance Energy Transfer (TR-FRET) Assay. Tb-anti-GST antibody was purchased from Invitrogen (Catalog No. PV4216).

### Probe Synthesis

### Reagents

All reagents were used as obtained from the vendor unless otherwise specified. Peptide synthesis reagents including diisopropylethylamine (DIEA), dichloromethane (DCM). N-methylpyrrolidone (NMP), 2-(1H-benzotriazole-1-yl)-1,1,3,3-tetramethyluronium hexafluorophosphate (HBTU), N-hydroxybenzotriazole (HOBt) and piperidine were obtained from Applied Biosystems, Inc. (ABI), Foster City, CA or American Bioanalytical, Natick, MA.

Preloaded 9-Fluorenylmethyloxycarbonyl (Fmoc) amino acid cartridges (Fmoc-Ala-OH, Fmoc-Cys(Trt)-OH, Fmoc-Asp(tBu)-OH, Fmoc-Glu(tBu)-OH, Fmoc-Phe-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Asn(Trt)-OH, Fmoc-Pro-OH, Fmor-Gln(Trt)-OH, Fmoc-Arg(Pbf)-OH, Fmoc-Ser(tBu)-OH, Fmoc-Thr(tBu)-OH, Fmoc-Val-OH, Fmoc-Trp(Boc)-OH, Fmoc-Tyr(tBu)-OH) were obtained from ABI or Anaspec, San Jose, CA.

The peptide synthesis resin (Fmoc-Rink amide MBHA resin) and Fmoc-Lys (Mtt)-OH were obtained from Novabiochem, San Diego, CA.

Single-isomer 6-carboxyfluorescein succinimidyl ester (6-FAM-NHS) was obtained from Anaspec.

Trifluoroacetic acid (TFA) was obtained from Oakwood Products, West Columbia, SC.

Thioanisole, phenol, triisopropylsilane (TIS), 3,6-dioxa-1,8-octanedithiol (DODT) and isopropanol were obtained from Aldrich Chemical Co., Milwaukee, WI.

Matrix-assisted laser desorption ionization mass-spectra (MALDI-MS) were recorded on an Applied Biosystems Voyager DE-PRO MS).

Electrospray mass-spectra (ESI-MS) were recorded on Finnigan SSQ7000 (Finnigan Corp., San Jose, CA) in both positive and negative ion mode.

### General Procedure for Solid-Phase Peptide Synthesis (SPPS)

Peptides were synthesized with, at most, 250 μmol preloaded Wang resin/vessel on an ABI 433A peptide synthesizer using 250 μmol scale Fastmoc™ coupling cycles. Preloaded cartridges containing 1 mmol standard Fmoc-amino acids, except for the position of attachment of the fluorophore, where 1 mmol Fmoc-Lys (Mtt)-OH was placed in the cartridge, were used with conductivity feedback monitoring. N-terminal acetylation was accomplished by using 1 mmol acetic acid in a cartridge under standard coupling conditions.

Removal of 4-Methyltrityl(Mtt) from Lysine

The resin from the synthesizer was washed thrice with dichloromethane and kept wet, 150 mL of 95:4:1 dichloromethane:triisopropylsilane:trifluoroacetic acid was flowed through the resin bed over 30 minutes. The mixture turned deep yellow then faded to pale yellow. 100 mL of N,N-dimethylformamide (DMF) was flowed through the bed over 15 minutes. The resin was then washed thrice with DMF and filtered. Ninhydrin tests showed a strong signal for primary amine.

Resin Labeling with 6-Carboxyfluorescein-NHS (6-FAM-NHS)

The resin was treated with 2 equivalents 6-FAM-NHS in 1%, DIEA/DMF and stirred or shaken at ambient temperature overnight. When complete, the resin was drained, washed thrice with DMF, thrice with (1×dichloromethane and 1×methanol) and dried to provide an orange resin that was negative by ninhydrin test.

### General Procedure for Cleavage and Deprotection of Resin-Bound Peptide

Peptides were cleaved from the resin by shaking for 3 hours at ambient temperature in a cleavage cocktail consisting of 80% TFA, 5% water, 5% thioanisole, 5% phenol, 2.5% TIS, and 2.5% EDT (1 mL/0.1 g resin). The resin was removed by filtration and rinsing twice with TFA. The TFA was evaporated from the filtrates, and product was precipitated with ether (10 mL/0.1 g resin), recovered by centrifugation, washed twice with ether (10 mL/0.1 g resin) and dried to give the crude peptide.

### General Procedure for Purification of Peptides

The crude peptides were purified on a Gilson preparative HPLC system running Unipoint® analysis software (Gilson, Inc., Middleton, WI) on a radial compression column containing two 25×100 mm segments packed with Delta-Pak™ C18 15 μm particles with 100 Å pore size and eluted with one of the gradient methods listed below. One to two milliliters of crude peptide solution (10 mg/mL in 90% DMSO/water) was purified per injection. The peaks containing the product(s) from each run were pooled and lyophilized. All preparative runs were run at 20 mL/minute with eluents as buffer A: 0.1% TFA-water and buffer B: acetonitrile.

### General Procedure for Analytical HPLC

Analytical HPLC was performed on a Hewlett-Packard 1200 series system with a diode-array detector and a Hewlett-Packard 1046A fluorescence detector running HPLC 3D ChemStation software version A.03.04 (Hewlett-Packard. Palo Alto, CA) on a 4.6×250 mm YMC column packed with ODS-AQ 5 μm particles with a 120 Å pore size and eluted with one of the gradient methods listed below after preequilibrating at the starting conditions for 7 minutes. Eluents were buffer A: 0.1% TFA-water and buffer B: acetonitrile. The flow rate for all gradients was 1 mL/minute.

### Synthesis of Probe F-Bak

Peptide probe F-bak, which binds MCL-1, was synthesized as described below. Probe F-Bak is acetylated at the N-terminus, amidated at the C-terminus and has the amino acid sequence GQVGRQLAIIGDKINR (SEQ ID NO:1). It is fluoresceinated at the lysine residue (K) with 6-FAM. Probe F-Bak can be abbreviated as follows: acetyl-GQVGRQLAIIGDK(6-FAM)INR-NH₂.

To make probe F-Bak. Fmoc-Rink amide MBHA resin was extended using the general peptide synthesis procedure to provide the protected resin-bound peptide (1.020 g). The Mtt group was removed, labeled with 6-FAM-NHS and cleaved and deprotected as described hereinabove to provide the crude product. This product was purified by RP-HPLC. Fractions across the main peak were tested by analytical RP-HPLC, and the pure fractions were isolated and lyophilized, with the major peak providing the title compound. MALDI-MS m/==2137.1 [(M+H)⁺].

### Alternative Synthesis of Peptide Probe F-Bak

In an alternative method, the protected peptide was assembled on 0.25 mmol Fmoc-Rink amide MBHA resin (Novabiochem) on an Applied Biosystems 433A automated peptide synthesizer running Fastmoc™ coupling cycles using pre-loaded 1 mmol amino acid cartridges, except for the fluorescein (6-FAM)-labeled lysine, where 1 mmol Fmoc-Lys (4-methyltrityl) was weighed into the cartridge. The N-terminal acetyl group was incorporated by putting 1 mmol acetic acid in a cartridge and coupling as described hereinabove. Selective removal of the 4-methyltrityl group was accomplished with a solution of 95:4:1 DCM:TIS:TFA (v/v/v) flowed through the resin over 15 minutes, followed by quenching with a flow of dimethylformamide. Single-isomer 6-carboxyfluorescein-NHS was reacted with the lysine side-chain in 1%, DIEA in DMF and confirmed complete by ninhydrin testing. The peptide was cleaved from the resin and side-chains deprotected by treating with 80:5:5:5:2.5:2.5 TFA/water/phenol/thioanisole/triisopropylsilane: 3,6-dioxa-1,8-octanedithiol (v/v/v/v/v/v), and the crude peptide was recovered by precipitation with diethyl ether. The crude peptide was purified by reverse-phase high-performance liquid chromatography, and its purity and identity were confirmed by analytical reverse-phase high-performance liquid chromatography and matrix-assisted laser-desorption mass-spectrometry (m/==2137.1 ((M+H)⁺).

### Time Resolved-Fluorescence Resonance Energy Transfer (TR-FRET) Assay

The ability of exemplary MCL-1 inhibitors Example 1 to Example 178 to compete with probe F-Bak for binding MCL-1 was demonstrated using a Time Resolved Fluorescence Resonance Energy Transfer (TR-FRET) binding assay.

### Method

For the assay, an acoustic dispenser was used to prepare dilution series from 10 mM test compounds in 100% DMSO and directly transfer 160 nL into low volume 384-well assay plates. 8 μL of a protein/probe/antibody mix was then added to each well resulting in final concentrations listed below:
Test compound: 11 three-fold dilutions beginning at 25 μM

| | | |
|---|---|---|
| Protein: | GST-MCL-1 | 1 nM |
| Antibody | Tb-anti-GST | 1 nM |
| Probe: | F-Bak | 100 nM |

The samples were then mixed on a shaker for 1 minute and incubated for an additional 2 hours at room temperature. For each assay plate, a probe/antibody and protein/antibody/probe mixture were included as a negative and a positive control, respectively. Fluorescence was measured on the Envision (Perkin Elmer) using a 340/35 nm excitation filter and 520/525 (F-Bak) and 495/510 nm (Tb-labeled anti-his antibody) emission filters. Dissociation constants (Kᵢ) were determined using Wang's equation (Wang, 1995, FEBS Lett. 360:111-114). The TR-FRET assay can be performed in the presence of varying concentrations of human serum (HS) or fetal bovine serum (FBS). Compounds were tested both without HS and in the presence of 10% HS.

### Results

The results of binding assays (Kᵢ in nanomolar) are provided in Table 2, below, and demonstrate the ability of compounds of the disclosure to bind MCL-1 protein.

TABLE 2
TR-FRET MCL-1 Binding Data

| | MCL-1 | MCL-1 |
|---|---|---|
| | Binding | Binding |
| | Kᵢ | Kᵢ |
| Example | (nM) | (nM, 10% HS) |
| 1 | 0.004 | 0.158 |
| 2 | 4.100 | 76.000 |
| 3 | 0.519 | 36.600 |
| 4 | 0.007 | 0.052 |
| 5 | 0.009 | 0.057 |
| 6 | 0.014 | 0.221 |
| 7 | 0.054 | 0.599 |
| 8 | 8.825 | 110.886 |
| 9 | 0.048 | 0.296 |
| 10 | 0.060 | 0.494 |
| 11 | 0.374 | 2.339 |
| 12 | 0.603 | 0.227 |
| 13 | 0.054 | 0.251 |
| 14 | 0.124 | 0.547 |
| 15 | 0.015 | 0.152 |
| 16 | 0.028 | 0.359 |
| 17 | 0.015 | 0.246 |
| 18 | 0.011 | 0.269 |
| 19 | 0.006 | 0.069 |
| 20 | 0.007 | 0.165 |
| 21 | 0.059 | 0.556 |
| 22 | 0.015 | 0.214 |
| 23 | 0.018 | 0.238 |
| 24 | 0.015 | 0.098 |
| 25 | 0.844 | 5.700 |
| 26 | 0.021 | 0.110 |
| 27 | 0.987 | 22.035 |
| 28 | 0.021 | 0.027 |
| 29 | 0.204 | 1.210 |
| 30 | 0.033 | 0.252 |
| 31 | 0.004 | 0.049 |
| 32 | 0.003 | 0.034 |
| 33 | 0.088 | 2.230 |
| 34 | 0.016 | 0.276 |
| 35 | 0.007 | 0.113 |
| 36 | 0.144 | 1.164 |
| 37 | 0.009 | 0.124 |
| 38 | 0.106 | 0.416 |
| 39 | 0.042 | 0.193 |
| 40 | 0.018 | 0.085 |
| 41 | 0.078 | 1.054 |
| 42 | 0.085 | 0.747 |
| 43 | 41.100 | 39.400 |
| 44 | 0.124 | 0.224 |
| 45 | 0.094 | 0.343 |
| 46 | 1.020 | 1.080 |
| 47 | 0.018 | 3.117 |
| 48 | 0.644 | 5.320 |
| 49 | 0.039 | 0.775 |
| 50 | 0.199 | 0.513 |
| 51 | 0.011 | 0.076 |
| 52 | 0.348 | 3.305 |
| 53 | 0.301 | 0.632 |
| 54 | 0.025 | 0.139 |
| 55 | 38.200 | 92.600 |
| 56 | 0.157 | 1.180 |
| 57 | 0.013 | 0.072 |
| 58 | 0.050 | 0.161 |
| 59 | 0.047 | 0.122 |
| 60 | 0.042 | 0.125 |
| 61 | 0.364 | 4.198 |
| 62 | 0.324 | 2.260 |
| 63 | 0.039 | 0.148 |
| 64 | 0.038 | 0.167 |
| 65 | 0.195 | 0.650 |
| 66 | 0.015 | 0.119 |
| 67 | 0.016 | 0.073 |
| 68 | 0.031 | 0.209 |
| 69 | 0.012 | 0.053 |
| 70 | 0.012 | 0.083 |
| 71 | 0.039 | 0.106 |
| 72 | 0.064 | 0.184 |
| 73 | 0.037 | 0.208 |
| 74 | 0.083 | 0.120 |
| 75 | 0.375 | 2.670 |
| 76 | 0.160 | 0.634 |
| 77 | 0.089 | 0.325 |
| 78 | 2.281 | 16.000 |
| 79 | 0.088 | 1.000 |
| 80 | 0.107 | 0.797 |
| 81 | 0.083 | 0.417 |
| 82 | 0.014 | 0.135 |
| 83 | 0.034 | 0.154 |
| 84 | 0.033 | 0.232 |
| 85 | 0.029 | 0.191 |
| 86 | 0.008 | 0.102 |
| 87 | 0.004 | 0.068 |
| 88 | 0.033 | 0.143 |
| 89 | 0.067 | 0.485 |
| 90 | 0.044 | 3.410 |
| 91 | 0.231 | 2.100 |
| 92 | 0.006 | 0.095 |
| 93 | 0.748 | 4.340 |
| 94 | 0.181 | 1.082 |
| 95 | 0.180 | 0.973 |
| 96 | 0.003 | 0.037 |
| 97 | 0.016 | 0.137 |
| 98 | 49.800 | 49.100 |
| 99 | 0.543 | 6.175 |
| 100 | 0.017 | 0.176 |
| 101 | 0.020 | 0.235 |
| 102 | 0.229 | 3.270 |
| 103 | 0.033 | 0.172 |
| 104 | 0.009 | 0.089 |
| 105 | 0.011 | 0.071 |
| 106 | 0.036 | 0.166 |
| 107 | 0.026 | 0.412 |
| 108 | 1.080 | 6.620 |
| 109 | 0.104 | 2.430 |
| 110 | 9.630 | 49.900 |
| 111 | 0.014 | 0.101 |
| 112 | 0.012 | 0.217 |
| 113 | 0.016 | 0.065 |
| 114 | 0.018 | 0.150 |
| 115 | 0.093 | 0.862 |
| 116 | 0.332 | 2.938 |
| 117 | 0.025 | 0.219 |
| 118 | 1.990 | 27.300 |
| 119 | 0.004 | 0.160 |
| 120 | 26.300 | 444.000 |
| 121 | 0.052 | 0.231 |
| 122 | 0.024 | 0.698 |
| 123 | 0.003 | 0.051 |
| 124 | 0.012 | 0.145 |
| 125 | 0.037 | 0.455 |
| 126 | 0.043 | 0.390 |
| 127 | 0.028 | 0.339 |
| 128 | 0.027 | 0.275 |
| 129 | 0.002 | 0.064 |
| 130 | 0.026 | 1.740 |
| 131 | 0.024 | 1.140 |
| 132 | 0.020 | 1.300 |
| 133 | 0.044 | 0.168 |
| 134 | 0.047 | 0.339 |
| 135 | 0.029 | 0.489 |
| 136 | 0.027 | 0.758 |
| 137 | 0.031 | 0.999 |
| 138 | 0.007 | 0.049 |
| 139 | 0.147 | 1.210 |
| 140 | 34.900 | 166.000 |
| 141 | 0.008 | 0.363 |
| 142 | 0.185 | 0.776 |
| 143 | 0.023 | 0.175 |
| 144 | 10.900 | >444 |
| 145 | 0.081 | 0.399 |
| 146 | 0.001 | 0.041 |
| 147 | 0.002 | 0.098 |
| 148 | 0.034 | 0.346 |
| 149 | 0.006 | 0.140 |
| 150 | 0.043 | 0.267 |
| 151 | 0.010 | 0.325 |
| 152 | 0.011 | 0.041 |
| 153 | 0.001 | 0.023 |
| 154 | 0.018 | 0.191 |
| 155 | 0.001 | 0.030 |
| 156 | 1.875 | 23.598 |
| 157 | 0.008 | 0.168 |
| 158 | 0.003 | 0.410 |
| 159 | 0.008 | 0.087 |
| 160 | 0.005 | 0.083 |
| 161 | 0.002 | 0.141 |
| 162 | 0.159 | 0.934 |
| 163 | 0.013 | 0.285 |
| 164 | 0.061 | 0.453 |
| 165 | 0.031 | 0.267 |
| 166 | 0.002 | 0.058 |
| 167 | 0.060 | 0.698 |
| 168 | 0.025 | 0.671 |
| 169 | 0.030 | 0.631 |
| 170 | 0.042 | 1.850 |
| 171 | 0.100 | 3.980 |
| 172 | 0.141 | 2.740 |
| 173 | NT | NT |
| 174 | 0.008 | 0.200 |
| 175 | <0.010 | 0.122 |
| 176 | 0.151 | 0.868 |
| 177 | NT | NT |
| 178 | NT | NT |

NT = not tested, NV = not valid

### Exemplary MCL-1 Inhibitors Demonstrate In Vitro Efficacy in Tumor Cell Viability Assays

The in vitro efficacy of exemplary MCL-1 inhibitors can be determined in cell-based killing assays using a variety of cell lines and mouse tumor models. For example, their activity on cell viability can be assessed on a panel of cultured tumorigenic and non-tumorigenic cell lines, as well as primary mouse or human cell populations. MCL-1 inhibitory activity of exemplary MCL-1 inhibitors was confirmed in a cell viability assay with AMO-1 and NCI-H929 human multiple myeloma tumor cell lines.

### Method

In one exemplary set of conditions, NCI-H929 or AMO-1 (ATCC, Manassas, VA) were plated 4,000 cells per well in 384-well tissue culture plates (Corning, Corning, NY) in a total volume of 25 μL RPMI tissue culture medium supplemented with 10% fetal bovine serum (Sigma-Aldrich, St. Louis, MO) and treated with a 3-fold serial dilution of the compounds of interest with a Labcyte Echo from a final concentration of 10 UM to 0.0005 μM. Each concentration was tested in duplicate at least 3 independent times. A luminescent signal proportional to the number of viable cells following 24 hours of compound treatment was determined using the CellTiter-Glo® Luminescent Cell Viability Assay according to the manufacturer's recommendations (Promega Corp., Madison, WI). The plates were read in a Perkin Elmer Envision using a Luminescence protocol. To generate dose response curves the data is normalized to percent viability by setting the averages of the staurosporine (10 μM) and DMSO only control wells to 0% and 100% viability respectively. The IC50 values for the compounds are generated by fitting the normalized data with Accelrys Assay Explorer 3.3 to a sigmoidal curve model using linear regression, Y=(100*xn)/(Kn+xn), where Y is the measured response, x is the compound concentration, n is the Hill Slope and K is the IC50 and the lower and higher asymptotes are constrained to 0 and 100 respectively.

### Results

The results of AMO-1 and H929 cell viability assays (IC₅₀ in nanomolar) carried out in the presence of 10% FBS for exemplary MCL-1 inhibitors are provided in Table 3, below. The results demonstrate the ability of compounds of the disclosure to potently inhibit the growth of human tumor cells in vitro.

TABLE 3
MCL-1 Inhibitor In Vitro Cell Efficacy Data

| | AMO-1 | H929 |
|---|---|---|
| | Viability | Viability |
| | IC₅₀ | IC₅₀ |
| EXAMPLE | (μM, 10% FBS) | (μM, 10% FBS) |
| 1 | 0.002 | 0.004 |
| 2 | 0.210 | 0.276 |
| 3 | 0.214 | 0.323 |
| 4 | 0.007 | 0.011 |
| 5 | 0.004 | 0.008 |
| 6 | 0.030 | 0.024 |
| 7 | 0.007 | 0.011 |
| 8 | 0.518 | 0.989 |
| 9 | 0.732 | >1.0 |
| 10 | >1.0 | >1.0 |
| 11 | 0.363 | 0.514 |
| 12 | 0.0187 | 0.0316 |
| 13 | 0.00046 | 0.001 |
| 14 | 0.00154 | 0.00258 |
| 15 | 0.000524 | 0.000751 |
| 16 | 0.000177 | 0.000347 |
| 17 | 0.0155 | 0.0298 |
| 18 | 0.00295 | 0.00531 |
| 19 | 0.0963 | 0.365 |
| 20 | 0.00425 | 0.00804 |
| 21 | 0.0143 | 0.0102 |
| 22 | 0.00133 | 0.0018 |
| 23 | 0.00222 | 0.00389 |
| 24 | 0.0385 | 0.157 |
| 25 | 0.0727 | 0.0808 |
| 26 | 0.00112 | 0.00289 |
| 27 | 0.314 | 0.319 |
| 28 | 0.00269 | 0.00583 |
| 29 | 0.000894 | 0.000942 |
| 30 | 0.00277 | 0.00356 |
| 31 | 0.00743 | 0.0133 |
| 32 | 0.00878 | 0.0143 |
| 33 | 0.000282 | 0.00123 |
| 34 | 0.00123 | 0.00242 |
| 35 | 0.010 | 0.017 |
| 36 | 0.32 | 0.653 |
| 37 | 0.0096 | 0.0167 |
| 38 | 0.00236 | 0.00577 |
| 39 | 0.00119 | 0.00353 |
| 40 | 0.0030 | 0.00474 |
| 41 | 0.0159 | 0.019 |
| 42 | 0.0011 | 0.00314 |
| 43 | >1.0 | >1.0 |
| 44 | 0.0211 | 0.0179 |
| 45 | 0.000461 | 0.000598 |
| 46 | 0.00027 | 0.000388 |
| 47 | 0.0182 | 0.0205 |
| 48 | 0.00275 | 0.00798 |
| 49 | 0.0222 | 0.0431 |
| 50 | 0.0676 | 0.103 |
| 51 | 0.0034 | 0.00671 |
| 52 | 0.000648 | 0.00269 |
| 53 | 0.0042 | 0.00571 |
| 54 | 0.000591 | 0.000771 |
| 55 | >1.0 | >1.0 |
| 56 | 0.0534 | 0.13 |
| 57 | 0.000244 | 0.00037 |
| 58 | 0.000378 | 0.00056 |
| 59 | 0.000686 | 0.000848 |
| 60 | 0.000987 | 0.00115 |
| 61 | 0.00886 | 0.0115 |
| 62 | 0.0251 | 0.095 |
| 63 | 0.00205 | 0.0032 |
| 64 | 0.00163 | 0.00243 |
| 65 | 0.0209 | 0.0596 |
| 66 | 0.000876 | 0.00121 |
| 67 | 0.0011 | 0.00176 |
| 68 | 0.000426 | 0.000938 |
| 69 | 0.000302 | 0.000615 |
| 70 | 0.000263 | 0.000489 |
| 71 | 0.00468 | 0.00851 |
| 72 | 0.000331 | 0.000743 |
| 73 | 0.00262 | 0.00556 |
| 74 | 0.00153 | 0.00237 |
| 75 | 0.00386 | 0.00914 |
| 76 | 0.000409 | 0.000648 |
| 77 | 0.000193 | 0.000405 |
| 78 | 0.000935 | 0.00552 |
| 79 | 0.000418 | 0.00163 |
| 80 | 0.000189 | 0.000954 |
| 81 | 0.000161 | 0.000687 |
| 82 | 0.000563 | 0.00109 |
| 83 | 0.00116 | 0.0026 |
| 84 | 0.000175 | 0.000528 |
| 85 | 0.000198 | 0.000485 |
| 86 | 0.000105 | 0.000258 |
| 87 | 0.0000959 | 0.000253 |
| 88 | 0.00117 | 0.00206 |
| 89 | 0.00262 | 0.00299 |
| 90 | 0.000183 | 0.000787 |
| 91 | 0.00072 | 0.00192 |
| 92 | 0.000223 | 0.00031 |
| 93 | 0.00553 | 0.00424 |
| 94 | 0.00203 | 0.00184 |
| 95 | 0.0039 | 0.00317 |
| 96 | 0.000287 | 0.000259 |
| 97 | 0.00060 | 0.000482 |
| 98 | 0.00399 | 0.0028 |
| 99 | 0.000655 | 0.00269 |
| 100 | 0.00033 | 0.000547 |
| 101 | 0.000195 | 0.000298 |
| 102 | 0.00029 | 0.000835 |
| 103 | 0.000281 | 0.000578 |
| 104 | 0.0000488 | 0.0000557 |
| 105 | 0.000192 | 0.000198 |
| 106 | 0.000216 | 0.000288 |
| 107 | 0.00147 | 0.000552 |
| 108 | 0.00343 | 0.00417 |
| 109 | 0.0166 | 0.0128 |
| 110 | 0.000306 | 0.00103 |
| 111 | 0.000161 | 0.000298 |
| 112 | 0.000132 | 0.000283 |
| 113 | 0.00028 | 0.00064 |
| 114 | 0.000197 | 0.000331 |
| 115 | 0.000593 | 0.00113 |
| 116 | 0.00297 | 0.00798 |
| 117 | 0.000205 | 0.000364 |
| 118 | 0.000247 | 0.000396 |
| 119 | 0.000181 | 0.000333 |
| 120 | 0.000231 | 0.000386 |
| 121 | 0.00508 | 0.00956 |
| 122 | 0.00107 | 0.00277 |
| 123 | 0.00128 | 0.00467 |
| 124 | 0.000355 | 0.000818 |
| 125 | 0.000288 | 0.00111 |
| 126 | 0.000544 | 0.00169 |
| 127 | 0.000124 | 0.000385 |
| 128 | 0.0000939 | 0.000313 |
| 129 | 0.000144 | 0.000562 |
| 130 | 0.000309 | 0.000489 |
| 131 | 0.000367 | 0.000585 |
| 132 | 0.000234 | 0.000266 |
| 133 | 0.00324 | 0.00879 |
| 134 | 0.00304 | 0.010 |
| 135 | 0.000191 | 0.000591 |
| 136 | 0.000403 | 0.000392 |
| 137 | 0.000394 | 0.000286 |
| 138 | 0.0020 | 0.00221 |
| 139 | 0.00231 | 0.00307 |
| 140 | 0.242 | 0.244 |
| 141 | 0.000441 | 0.000503 |
| 142 | 0.0000488 | 0.000156 |
| 143 | 0.000216 | 0.000203 |
| 144 | 0.001778 | 0.007699 |
| 145 | 0.000407 | 0.000973 |
| 146 | 0.002232 | 0.004076 |
| 147 | 0.000204 | 0.000162 |
| 148 | 0.000180 | 0.000736 |
| 149 | 0.000363 | 0.001400 |
| 150 | 0.001508 | 0.002526 |
| 151 | 0.000303 | 0.000735 |
| 152 | 0.007316 | 0.005929 |
| 153 | 0.007283 | 0.006879 |
| 154 | 0.000698 | 0.000643 |
| 155 | 0.000188 | 0.000187 |
| 156 | 0.004535 | 0.011117 |
| 157 | 0.000233 | 0.000821 |
| 158 | 0.000258 | 0.001310 |
| 159 | 0.000296 | 0.000379 |
| 160 | 0.000116 | 0.000206 |
| 161 | 0.000262 | 0.000502 |
| 162 | 0.012000 | 0.014300 |
| 163 | 0.000399 | 0.001340 |
| 164 | 0.000416 | 0.001470 |
| 165 | 0.003530 | 0.008560 |
| 166 | 0.000927 | 0.003200 |
| 167 | 0.002000 | 0.008920 |
| 168 | NT | NT |
| 169 | 0.000306 | 0.001150 |
| 170 | 0.000646 | 0.002780 |
| 171 | 0.001120 | 0.007570 |
| 172 | 0.000966 | 0.005240 |
| 173 | 0.001140 | 0.004520 |
| 174 | NT | NT |
| 175 | 0.001640 | 0.005730 |
| 176 | 0.003860 | 0.007300 |
| 177 | 0.004220 | 0.009290 |
| 178 | 0.152 | 0.37 |

NT = not tested, NV = not valid

The ability of certain exemplary compounds of the present disclosure to inhibit the growth of tumor cells in mice was demonstrated in xenograft models derived from a human multiple myeloma cell line, AMO-1.

### Evaluation of Efficacy in Xenograft Models Methods

AMO-1 cells were obtained from the Deutsche Sammlung von Microorganismen und Zellkulturen (DSMZ, Braunschweig, Germany). The cells were cultured as monolayers in RPMI-1640 culture media (Invitrogen, Carlsbad, CA) that was supplemented with 10% Fetal Bovine Serum (FBS, Hyclone, Logan, UT). To generate xenografts, 5×10⁶ viable cells were inoculated subcutaneously into the right flank of immune deficient female SCID/bg mice (Charles River Laboratories, Wilmington, MA) respectively. The injection volume was 0.2 mL and composed of a 1:1 mixture of S MEM and Matrigel (BD, Franklin Lakes, NJ). Tumors were size matched at approximately 200 mm³. MCL-1 inhibitors were formulated in 5% DMSO, 20% cremaphor EL and 75% DSW for injection and injected intraperitoneally. Injection volume did not exceed 200 μL. Alternatively, MCL-1 inhibitors were formulated in 5% DMSO, 10% cremaphor and 85% DSW for injection and injected intravenously. Injection volume did not exceed 200 L. Therapy began within 24 hours after size matching of the tumors. Mice weighed approximately 21 g at the onset of therapy. Tumor volume was estimated two to three times weekly. Measurements of the length (L) and width (W) of the tumor were taken via electronic caliper and the volume was calculated according to the following equation: V=L×W²/2. Mice were euthanized when tumor volume reached 3,000 mm³ or skin ulcerations occurred. Seven or eight mice were housed per cage. Food and water were available ad libitum. Mice were acclimated to the animal facilities for a period of at least one week prior to commencement of experiments. Animals were tested in the light phase of a 12-hour light: 12-hour dark schedule (lights on at 06:00 hours).

To refer to efficacy of therapeutic agents, parameters of amplitude (TGIₘₐₓ), durability (TGD) of therapeutic response are used. TGIₘₐₓ is the maximum tumor growth inhibition during the experiment. Tumor growth inhibition is calculated by 100*(1−Tᵥ/Cᵥ) where Tᵥ and Cᵥ are the mean tumor volumes of the treated and control groups, respectively. TGD or tumor growth delay is the extended time of a treated tumor needed to reach a volume of 1 cm³ relative to the control group. TGD is calculated by 100*(T/C₁-1) where Tₜ and Cₜ are the median time periods to reach 1 cm³ of the treated and control groups, respectively.

### Results

As shown in Tables 4-6, compounds of the present disclosure are efficacious in an in vivo AMO-1 xenograft model of multiple myeloma, showing significant tumor growth inhibition and tumor growth delay after intraperitoneal (IP) or intravenous (IV) doses of drug.

TABLE 4
In vivo efficacy of MCL-1 inhibitors in AMO-1 Xenograft Model

| | Dose | | TGIₘₐₓ | TGD |
|---|---|---|---|---|
| Treatment | (mg/kg/day) | Route/Regimen | (%) | (%) |
| Vehicle | 0 | IP^{(a)}/QDx1 | 0 | 0 |
| Example 5 | 25 | IP/QDx1 | 55* | 17* |
| Example 4 | 25 | IP/QDx1 | 54* | 33* |
| Example 1 | 25 | IP/QDx1 | 91* | >92* |

^{(a)}IP formulation = 5% DMSO, 20% cremophor EL, 75% D5W
*p < 0.05 as compared to control treatment
7 mice per treatment group

TABLE 5
In vivo efficacy of MCL-1 inhibitors in AMO-1 Xenograft Model

| | Dose | | TGIₘₐₓ | TGD |
|---|---|---|---|---|
| Treatment | (mg/kg/day) | Route/Regimen | (%) | (%) |
| Vehicle | 0 | IP^{(a)}/QDx1 | 0 | 0 |
| Example 84 | 25 | IP/QDx1 | 71* | 33* |
| Example 87 | 25 | IP/QDx1 | 99* | 158* |

^{(a)}IP formulation = 5% DMSO, 20% cremophor EL, 75% D5W
*p < 0.05 as compared to control treatment
7 mice per treatment group

TABLE 6
In vivo efficacy of MCL-1 inhibitors in AMO-1 Xenograft Model

| | Dose | | TGIₘₐₓ | TGD |
|---|---|---|---|---|
| Treatment | (mg/kg/day) | Route/Regimen | (%) | (%) |
| Vehicle | 0 | IV^{(a)}/QDx1 | 0 | 0 |
| Example 96 | 6.25 | IV/QDx1 | 66* | 67* |
| Example 142 | 6.25 | IV/QDx1 | 75* | 67* |

^{(a)}IP formulation = 5% DMSO, 10% cremophor EL, 85% D5W
*p < 0.05 as compared to control treatment
7 mice per treatment group

It is understood that the foregoing detailed description and accompanying examples are merely illustrative and are not to be taken as limitations upon the scope of the present disclosure, which is defined solely by the appended claims and their equivalents. Various changes and modifications to the disclosed embodiments will be apparent to those skilled in the art. All publications, patents, and patent applications cited herein are hereby incorporated by reference in their entirety for all purposes.

We claim:

1. A compound of Formula (I) or a pharmaceutically acceptable salt thereof, 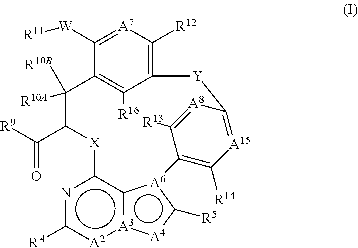
A² is CR², A³ is N, A⁴ is CR^{4a}, and A⁶ is C; or
A² is CR², A³ is N, A⁴ is O or S, and A⁶ is C; or
A² is N, A³ is C, A⁴ is O or S and A⁶ is C; or
A² is N, A³ is C, A⁴ is CR^{4a}, and A⁶ is N;
R^{A} is hydrogen, CH₃, halogen, CN, CH₂F, CHF₂, or CF₃;
X is O, or N(R^{x2}), wherein R^{x2} is hydrogen, C₁-C₃ alkyl, or unsubstituted cyclopropyl;
Y is (CH₂)ₘ, —CH═CH—(CH₂)ₙ—, —(CH₂)ₚ—CH═CH—, or —(CH₂)_{q}—CH═CH—(CH₂)ᵣ—; wherein 0, 1, 2, or 3 CH₂ groups are each independently replaced by O, N(R^{ya}), C(R^{ya})(R^{yb}), C(O), NC(O)R^{ya}, or S(O)₂;
m is 2, 3, 4, or 5;
n is 1, 2, or 3;
p is 1, 2, or 3;
q is 1 or 2; and
r is 1 or 2; wherein the sum of q and r is 2 or 3;
R^{ya}, at each occurrence, is independently hydrogen, C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; and
R^{yb} is C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or
R^{ya} and R^{yb}, together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1, 2, or 3 independently selected R⁸ groups;
R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{yh}, —SR^{yh}, —SO₂R^{yh}, and —N(R^{yi})(R^{yk});
G¹, at each occurrence, is a 4-11 membered heterocycle; wherein each G¹ is optionally substituted with 1, 2, or 3 substituents independently selected from the group consisting of G², —(C₁-C₆ alkylenyl)-G², -L^{1A}-(C₁-C₆ alkylenyl)ₛ-R^{x1}, and R⁸;
G², at each occurrence, is a C₃-C₇ monocyclic cycloalkyl, C₄-C₇ monocyclic cycloalkenyl, or a 4-11 membered heterocycle; wherein each G² is optionally substituted with 1 independently selected R^{t} groups;
L^{1A} is bond, O, N(H), N(C₁-C₆ alkyl), N[(C₁-C₆ alkyl)-R^{x1}], S, S(O), or S(O)₂, C(O)NH, C(O)N(C₁-C₆ alkyl), or C(O)N[(C₁-C₆ alkyl)-R^{x1}];
R² is independently hydrogen, halogen, CH₃, or CN;
R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄ alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups;
R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³;
G³, at each occurrence, is independently C₆-C₁₀ aryl, 5-11 membered heteroaryl, C₃-C₁₁ cycloalkyl, C₄-C₁₁ cycloalkenyl, or 4-7 membered heterocycle; wherein each G³ is optionally substituted with 1, 2, or 3 R^{v} groups;
A⁷ is N or CR⁷;
A⁸ is N or CR⁸;
A¹⁵ is N or CR¹⁵;
R¹, R¹² and R¹⁶ are each independently hydrogen, halogen, C₁-C₄ alkyl, C₁-C₄ haloalkyl, —CN, —OR^{7a}, —SR^{7a}, or —N(R^{7b})(R^{7c});
R⁸, R¹³, R¹⁴, and R¹⁵, are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; or
R⁸ and R¹³ are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and
R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8a})(R^{8c});
R⁹ is —OH, —O—C₁-C₄ alkyl, —O—CH₂—OC(O)(C₁-C₆ alkyl), —NHOH, 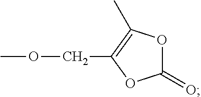
or —N(H)S(O)₂—(C₁-C₆ alkyl);
R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl; wherein the cyclopropyl is optionally substituted with one or two substituents independently selected from the group consisting of halogen, C₁-C₃ alkyl, and C₁-C₃ haloalkyl;
W is —CH═CH—, C₁-C₄ alkyl, -L¹-CHF—, -L¹-CH₂—, or —CH₂-L¹-; wherein L¹ at each occurrence, is independently O, S, S(O), S(O)₂, S(O)₂N(H), N(H), or N(C₁-C₃ alkyl);
R¹¹ is a C₆-C₁₀ aryl or a 5-11 membered heteroaryl; wherein each R¹¹ is optionally substituted with 1, 2, or 3 independently selected R^{w} groups;
R^{w}, at each occurrence, is independently C₁-C₆ alkyl, C₂-C₆ alkenyl, C₂-C₆ alkynyl, halogen, C₁-C₆ haloalkyl, —CN, NO₂, —OR^{11a}, —SR^{11b}, —S(O)₂R^{11b}, —S(O)₂N(R^{11c})₂, —C(O)R^{11a}, —C(O)N(R^{11c})₂, —N(R^{11c})₂, —N(R^{11c})C(O)R^{11b}, —N(R^{11c})S(O)₂R^{11b}, —N(R^{11c})C(O)O(R^{11b}), —N(R^{11c})C(O)N(R^{11c})₂, G⁴, —(C₁-C₆ alkylenyl)-OR^{11b}, —(C₁-C₆ alkylenyl)-OC(O)N(R^{11c})₂, —(C₁-C₆ alkylenyl)-SR^{11a}, —(C₁-C₆ alkylenyl)-S(O)₂R^{11b}, —(C₁-C₆ alkylenyl)-S(O)₂N(R^{11b})₂, —(C₁-C₆ alkylenyl)-C(O)R^{11a}, —(C₁-C₆ alkylenyl)-C(O)N(R^{11c})₂, —(C₁-C₆ alkylenyl)-N(R^{11c})₂, —(C₁-C₆ alkylenyl)-N(R^{11c})C(O)R^{11b}, —(C₁-C₆ alkylenyl)-N(R^{11c})S(O)₂R^{11b}, —(C₁-C₆ alkylenyl)-N(R^{11c}) C(O)O(R^{11b}), —(C₁-C₆ alkylenyl)-N(R^{11c})C(O)N(R^{11b})₂, —(C₁-C₆ alkylenyl)-CN, —N(C₁-C₆ alkylenyl)₂-G⁴, or (C₁-C₆ alkylenyl)-G⁴;
R^{11a} and R^{11c}, at each occurrence, are each independently hydrogen, C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11e})₂, or —(C₂-C₆ alkylenyl)-G⁴;
R^{11b}, at each occurrence, is independently C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11d})₂, or —(C₂-C₆ alkylenyl)-G⁴;
G⁴, at each occurrence, is independently R^{x1}, phenyl, monocyclic heteroaryl, C₃-C₁₁ cycloalkyl, C₄-C₁₁ cycloalkenyl, or 4-11 membered heterocycle; wherein each phenyl, monocyclic heteroaryl, C₃-C₁₁ cycloalkyl, C₄-C₁₁ cycloalkenyl, and 4-11 membered heterocycle is optionally substituted with 1, 2, 3, or 4 substituents independently selected from the group consisting of G⁵, R^{y}, —(C₁-C₆ alkylenyl)-G⁵, -L³-(C₁-C₆ alkylenyl)ₛ-R^{x1}, —(C₁-C₆ alkylenyl)ₛ-L³-(C₁-C₆ alkylenyl)ₛ-R^{x1}, -L³-(C₃-C₇ cycloalkyl)-R^{x1}, -L³-(C₄-C₇ cycloalkenyl)-R^{x1}, -L³-(4-7 membered heterocycle)-R^{x1}, and -L²-(C₁-C₆ alkylenyl)ₛ-G⁵;
L² is O, C(O), N(H), N(C₁-C₆ alkyl), NHC(O), C(O)O, S, S(O), or S(O)₂;
L³ is bond, O, C(O), N(H), N(C₁-C₆ alkyl), NHC(O), N(C₁-C₆ alkyl) C(O), N[(C₁-C₆ alkyl)ₛ-R^{x1}], N[(C₁-C₆ alkyl)ₛ-R^{x1}]C(O), S, S(O), or S(O)₂, C(O)NH, C(O)N(C₁-C₆ alkyl), or C(O)N[(C₁-C₆ alkyl)ₛ-R^{x1}];
s, at each occurrence, is independently is 0 or 1;
G⁵, at each occurrence, is independently phenyl, monocyclic heteroaryl, C₃-C₇ monocyclic cycloalkyl, C₄-C₇ monocyclic cycloalkenyl, or 4-12 membered heterocycle; wherein each G⁵ is optionally substituted with 1 independently selected R^{z} groups;
R^{s}, R^{t}, R^{u}, R^{v}, R^{y}, and R^{z}, at each occurrence, are each independently C₁-C₆ alkyl, C₂-C₆ alkenyl, C₂-C₆ alkynyl, halogen, C₁-C₆ haloalkyl, —CN, oxo, NO₂, P(O)(R^{k})₂, —OR^{m}, —OC(O)R^{k}, —OC(O)N(R^{j})₂, —SR^{j}, —S(O)₂R^{k}, —S(O)₂N(R^{j})₂, —C(O)R^{j}, —C(O)N(R^{j})₂, —N(Rⱼ)₂, —N(R^{j})C(O)R^{k}, —N(R^{j})S(O)₂R^{k}, —N(R^{j})C(O)O(R^{k}), —N(R^{j})C(O)N(R^{j})₂, —(C₁-C₆ alkylenyl)-OR^{j}, —(C₁-C₆ alkylenyl)-OC(O)N(R^{j})₂, —(C₁-C₆ alkylenyl)-SR^{j}, —(C₁-C₆ alkylenyl)-S(O)₂R^{k}, —(C₁-C₆ alkylenyl)-S(O)₂N(R^{j})₂, —(C₁-C₆ alkylenyl)-C(O)R^{j}, —(C₁-C₆ alkylenyl)-C(O)N(R^{j})₂, —(C₁-C₆ alkylenyl)-C(O)N(R^{j})S(O)₂R^{k}, —(C₁-C₆ alkylenyl)-N(R^{j})₂, —(C₁-C₆ alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, or —(C₁-C₆ alkylenyl)-CN;
R^{m} is hydrogen, C₁-C₆ alkyl, C₁-C₆ haloalkyl, —(C₂-C₆ alkylenyl)-OR^{j}, or —(C₂-C₆ alkylenyl)-N(R^{j})₂;
R^{yh}, R^{yi}, R^{yk}, R^{7a}, R^{7b}, R^{7c}, R^{8a}, R^{8b}, R^{8c}, R^{11d}, R^{11e}, and R^{j}, at each occurrence, are each independently hydrogen, C₁-C₆ alkyl, or C₁-C₆ haloalkyl;
R^{x1}, at each occurrence, is independently selected from the group consisting of a polyethylene glycol, a polyol, a polyether, CH₂P(O)(R^{k})₂, C(O)OH, S(O)(═NH) (C₁-C₃ alkyl), a carboxylic acid isostere, C₃-C₁₁ cycloalkyl, C₄-C₁₁ cycloalkenyl, or 4-11 membered heterocycle wherein the C₃-C₁₁ cycloalkyl, C₄-C₁₁ cycloalkenyl, and 4-11 membered heterocycle are substituted with two or more ORⁿ groups and optionally substituted with 1 independently selected R^{z} group, 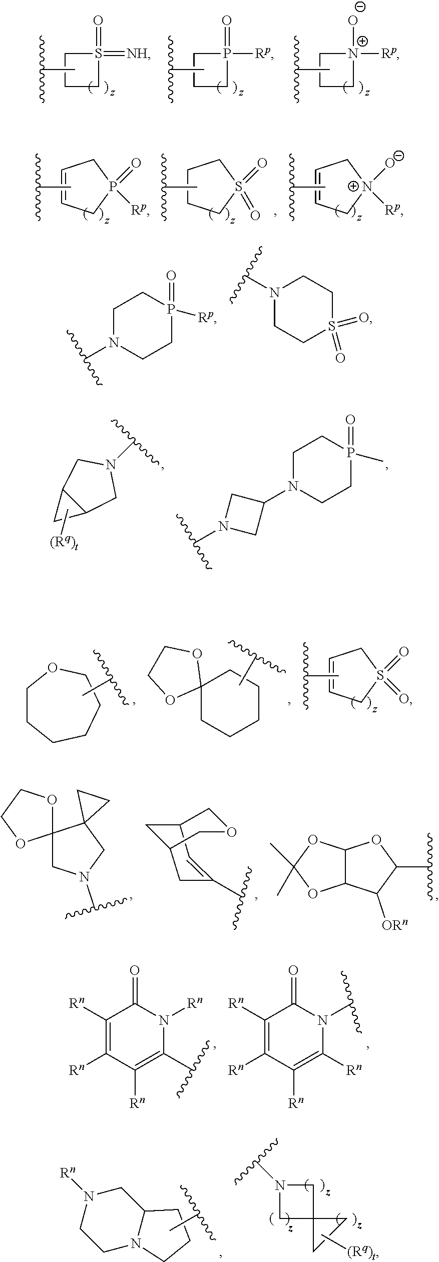 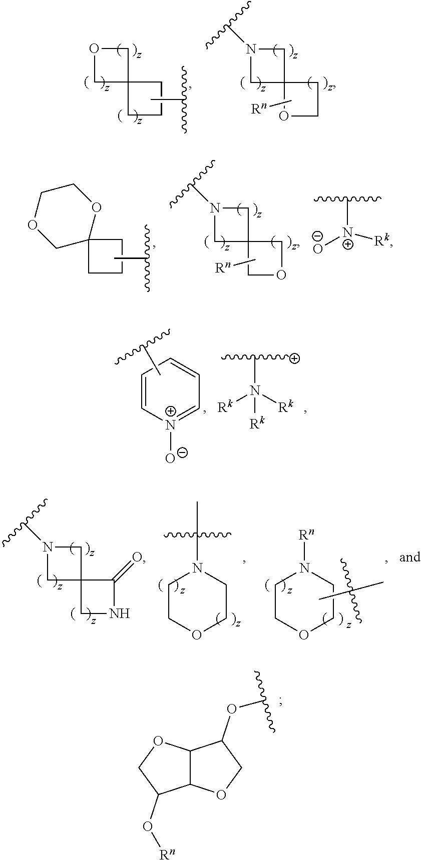
R^{k}, at each occurrence, is independently C₁-C₆ alkyl or C₁-C₆ haloalkyl;
Rⁿ, at each occurrence, is independently hydrogen, or C₁-C₆ alkyl;
R^{p} is C₁-C₃ alkyl, or cyclopropyl;
R^{q}, at each occurrence, is independently C(O)OH, halogen, —O—C₁-C₆ alkyl, or C₁-C₆ alkyl;
t is 0, 1, or 2; and
z, at each occurrence, is independently 1, 2, 3, or 4;
wherein at least one R^{x1} is present.

2. The compound of claim 1, or a pharmaceutically acceptable salt thereof, wherein R^{A} is hydrogen.

3. The compound of claim 1, or a pharmaceutically acceptable salt thereof, wherein R⁹ is —OH.

4. The compound of claim 1, or a pharmaceutically acceptable salt thereof, wherein R^{10A} and R^{10B}, are each independently hydrogen.

5. The compound of claim 1, or a pharmaceutically acceptable salt thereof, wherein R⁷, R¹² and R¹⁶ are each independently hydrogen.

6. The compound of claim 1, or a pharmaceutically acceptable salt thereof, wherein X is O.

7. The compound of claim 1, or a pharmaceutically acceptable salt thereof, wherein
R^{A} is hydrogen;
X is O;
R⁹ is —OH;
R^{10A} and R^{10B} are each independently hydrogen; and
R⁷, R¹² and R¹⁶ are each independently hydrogen.

8. The compound of claim 7, or a pharmaceutically acceptable salt thereof, wherein
A² is CH;
A³ is N;
A⁴ is CH; and
A⁶ is C.

9. The compound of claim 7, or a pharmaceutically acceptable salt thereof, wherein
A² is N;
A³ is C;
A⁴ is O; and
A⁶ is C.

10. The compound of claim 7 or a pharmaceutically acceptable salt thereof, wherein
A² is N;
A³ is C;
A⁴ is S; and
A⁶ is C.

11. The compound of claim 10, or a pharmaceutically acceptable salt thereof, wherein Y is (CH₂) m; wherein 1 CH₂ group is independently replaced by N(R^{ya}); and
m is 3.

12. The compound of claim 10 or a pharmaceutically acceptable salt thereof, wherein Y is (CH₂)ₘ; wherein 2 CH₂ groups are each independently replaced by O and 1 CH₂ group is replaced by C(R^{ya})(R^{yb}); and
m is 4.

13. The compound of claim 11, or a pharmaceutically acceptable salt thereof, wherein G¹ is piperazinyl substituted with 1 R^{s}.

14. The compound of claim 12, or a pharmaceutically acceptable salt thereof, wherein G¹ is piperazinyl substituted with 1 R^{s}.

15. The compound of claim 13 or a pharmaceutically acceptable salt thereof, wherein W is -L¹-CH₂—; and
L¹ is independently O.

16. The compound of claim 14 or a pharmaceutically acceptable salt thereof, wherein W is -L¹-CH₂—; and
L¹ is independently O.

17. The compound of claim 16 or a pharmaceutically acceptable salt thereof, wherein W is —O—CH₂—, and
R¹¹ is pyrimidinyl, optionally substituted with 1, 2, or 3 independently selected R^{w} groups.

18. The compound of claim 17 or a pharmaceutically acceptable salt thereof, wherein G⁴, at each occurrence, is independently phenyl substituted with 1 -L³-(C₁-C₆ alkylenyl)ₛ-R^{x1};
L³ is bond or O;
s, at each occurrence, is independently is 0 or 1;
R^{x1}, at each occurrence, is independently selected from the group consisting of a polyethylene glycol, or 4-11 membered heterocycle wherein the 4-11 membered heterocycle is substituted with two or more ORⁿ groups, and
Rⁿ is hydrogen or C₁-C₆ alkyl.

19. The compound of claim 1 or a pharmaceutically acceptable salt thereof, wherein the compound is selected from the group consisting of Example 1 to Example 178 of Table 1.

20. A pharmaceutical composition comprising a therapeutically effective amount of a compound of Formula (I) according to claim 1, or a pharmaceutically acceptable salt thereof, in combination with a pharmaceutically acceptable carrier.

21. A method for treating multiple myeloma in a subject comprising administering a therapeutically effective amount of a compound of Formula (I) according to claim 1 or a pharmaceutically acceptable salt thereof, to a subject in need thereof.


SEQUENCE LISTING (1 sequence)

SEQ ID NO: 1 - protein, 16 aa, synthetic construct
GQVGRQLAIIGDKINR